(19) United States
(12) Patent Application Publication
(10) Pub. No.: US 2015/0071923 A1
(43) Pub. Date: Mar. 12, 2015

(54) MODIFIED ANTI-EPIDERMAL GROWTH FACTOR RECEPTOR ANTIBODIES AND METHODS OF USE THEREOF

(71) Applicants: Ge Wei, San Diego, CA (US); Gregory I. Frost, Del Mar, CA (US); H. Michael Shepard, San Diego, CA (US); Christopher D. Thanos, Tiburon, CA (US)

(72) Inventors: Ge Wei, San Diego, CA (US); Gregory I. Frost, Del Mar, CA (US); H. Michael Shepard, San Diego, CA (US); Christopher D. Thanos, Tiburon, CA (US)

(21) Appl. No.: 14/485,620

(22) Filed: Sep. 12, 2014

Related U.S. Application Data
(60) Provisional application No. 61/960,253, filed on Sep. 12, 2013.

(51) Int. Cl. C07K 16/28; A61K 47/48; C07K 16/30

(52) U.S. Cl. CPC C07K 16/2863; C07K 16/30; C07K 16/3015; A61K 47/48384; A61K 47/48669; A61K 47/48584; C07K 2317/56; C07K 2317/24; C07K 2317/76; C07K 2317/55; C07K 2317/54; C07K 2317/622; C07K 2317/624; C07K 2317/626; C07K 2317/51; C07K 2317/515; C07K 2317/92
USPC 424/133.1; 530/387.3; 530/391.1; 530/391.7; 530/391.3; 530/391.9; 536/23.53; 424/178.1; 435/320.1; 435/69.6; 435/328

(57) ABSTRACT

Provided herein are modified anti-EGFR antibodies and nucleic acid molecules encoding modified anti-EGFR antibodies. Also provided are methods of treatment and uses using modified anti-EGFR antibodies.

### RELATED APPLICATIONS

Benefit of priority is claimed to U.S. Provisional Application Ser. No. 61/960,253, entitled “MODIFIED ANTI-EPIDERMAL GROWTH FACTOR RECEPTOR ANTIBODIES AND METHODS OF USE THEREOF,” filed Sep. 12, 2013.

This application is related to International PCT Application No. [Attorney Dkt. No. 33320-3118PC], filed the same day herewith, entitled “MODIFIED ANTI-EPIDERMAL GROWTH FACTOR RECEPTOR ANTIBODIES AND METHODS OF USE THEREOF,” which claims priority to U.S. Provisional Application Ser. No. 61/960,253.

This application also is related to U.S. application Ser. No. 13/815,553, filed Mar. 8, 2013, entitled “CONDITIONALLY ACTIVE ANTI-EPIDERMAL GROWTH FACTOR RECEPTOR ANTIBODIES AND METHODS OF USE THEREOF,” which claims priority to U.S. Provisional Application Ser. No. 61/685,089, entitled “CONDITIONALLY ACTIVE ANTI-EPIDERMAL GROWTH FACTOR RECEPTOR ANTIBODIES AND METHODS OF USE THEREOF,” filed Mar. 12, 2012.

This application also is related to International PCT Application Serial No. PCT/US13/30055, filed Mar. 8, 2013, entitled “CONDITIONALLY ACTIVE ANTI-EPIDERMAL GROWTH FACTOR RECEPTOR ANTIBODIES AND METHODS OF USE THEREOF,” which claims priority to U.S. Provisional Application Ser. No. 61/685,089, entitled “CONDITIONALLY ACTIVE ANTI-EPIDERMAL GROWTH FACTOR RECEPTOR ANTIBODIES AND METHODS OF USE THEREOF,” filed Mar. 12, 2012.

This application also is related to U.S. application Ser. No. 13/200,666, filed Sep. 27, 2011, entitled “METHODS FOR ASSESSING AND IDENTIFYING OR EVOLVING CONDITIONALLY ACTIVE THERAPEUTIC PROTEINS,” which is a continuation-in-part of International Application No. PCT/US11/50891, filed on Sep. 8, 2011, entitled “METHODS FOR ASSESSING AND IDENTIFYING OR EVOLVING CONDITIONALLY ACTIVE THERAPEUTIC PROTEINS,” which claims priority to U.S. Provisional Application Ser. No. 61/402,979, entitled “METHODS FOR ASSESSING AND IDENTIFYING OR EVOLVING CONDITIONALLY ACTIVE THERAPEUTIC PROTEINS AND CONDITIONALLY ACTIVE THERAPEUTIC PROTEINS,” filed Sep. 8, 2010.

The subject matter of each of the above-noted applications is incorporated by reference in its entirety.

### INCORPORATION BY REFERENCE OF SEQUENCE LISTING FILED ELECTRONICALLY

An electronic version of the Sequence Listing is filed herewith, the contents of which are incorporated by reference in their entirety. The electronic file was created on Sep. 12, 2014, is 744 kilobytes in size, and is titled 3118seq001.txt.

### FIELD OF THE INVENTION

Provided herein are modified, conditionally active anti-EGFR antibodies and nucleic acid molecules encoding modified, conditionally active anti-EGFR antibodies.

### BACKGROUND

Anti-EGFR antibodies are used in the clinical setting to treat and diagnose human diseases, for example cancer. For example, exemplary therapeutic antibodies include Cetuximab. Cetuximab is approved for the treatment of recurrent or metastatic head and neck cancer, colorectal cancer and other diseases and conditions. It can also be used in the treatment of other diseases or conditions involving overexpression of EGFR or aberrant signaling or activation of EGFR. Administered anti-EGFR antibodies can bind to EGFR in healthy cells and tissue. This limits the dosages that can be administered. Hence, Cetuximab and other anti-EGFR antibodies exhibit limitations when administered to patients. Accordingly, it is among the objects herein to provide improved anti-EGFR antibodies that exhibit increased EGFR binding activity in a tumor microenvironment compared to in a non-tumor environment.

### SUMMARY

Provided herein are modified anti-epidermal growth factor receptor (EGFR) antibodies, and antigen-binding fragments thereof. In particular, the antibodies and antigen binding fragments thereof include an amino acid replacement compared to the anti-EGFR antibody cetuximab or antigen-binding fragment thereof and other cetuximab variants and antigen-binding fragments. The antibodies contain an amino acid replacement in the variable heavy chain corresponding to replacement with glutamic acid (E) at the position corresponding to position 104 with reference to amino acid positions set forth in SEQ ID NO: 2 or 7. The modified anti-EGFR antibodies provided herein specifically bind to EGFR antigen (e.g., human EGFR) and soluble fragments thereof and exhibit greater activity (binding affinity) under conditions of acidic pH, such as is present in a tumor microenvironment, than under conditions of neutral pH, such as exists in non-tumor tissue, such as that which exists in the basal layer of the skin, which has a neutral pH of a about 7 to 7.2. Hence provided are modified cetuximab antibodies and fragments thereof that contain the amino acid replacement glutamic acid at a position corresponding to position 104. These include cetuximab, and modified variants of cetuximab and fragments thereof that contain additional modifications.

Anti-EGFR antibodies are employed as anti-tumor therapeutics because they bind to EGFR receptors and inhibit ligand binding, thereby preventing EGFR-mediated activities that occur upon ligand binding. As a result, such antibodies can inhibit or treat tumors. Because tissues, other than tumors, such as tissues in the skin, also express EGFRs, the anti-EGFR antibodies also inhibit activities of these receptors, thereby causing undesirable side-effects. The antibodies provided herein exhibit pH-selective binding activity, such that EGFR binding activity is reduced at neutral pH (e.g., pH 7.0 to 7.4) compared to antibodies that do not exhibit pH-selective binding activity and/or compared to EGFR binding activity under acidic pH conditions. By virtue of the pH-selective activity, the anti-EGFR antibodies provided produce fewer or lesser undesirable side-effects and/or exhibit improved efficacy in a treated subject by virtue of the ability to administer higher doses.

For example, provided herein are modified anti-EGFR antibodies, and antigen-binding fragments thereof, that contain an amino acid replacement(s) in a variable heavy chain of an unmodified anti-EGFR antibody, or antigen-binding fragment thereof, corresponding to replacement with glutamic acid (E) at a position corresponding to position 104 with reference to amino acid positions set forth in SEQ ID NO: 2 or 7, as long as the modified anti-EGFR antibody specifically binds epidermal growth factor receptor (EGFR) or a soluble fragment thereof; the unmodified anti-EGFR antibody is cetuximab, an antigen-binding fragment thereof or a variant thereof, specifically binds to EGFR and does not already contain the amino acid replacement; and corresponding amino acid positions are identified by alignment of the variable heavy chain of the antibody with the variable heavy chain set forth in SEQ ID NO: 2 or 7.

In any of such examples of the modified anti-EGFR antibodies provided herein, the unmodified anti-EGFR antibody, or antigen-binding fragment thereof, to which the amino acid replacement is made contains a variable heavy chain set forth in SEQ ID NO: 2 or 7, or a sequence of amino acids that exhibits at least 70% sequence identity to SEQ ID NO: 2 or 7; and a variable light chain set forth in SEQ ID NO: 4, 9 or 11 or a sequence of amino acids that exhibits at least 70% sequence identity to SEQ ID NO: 4, 9 or 11. For example, the unmodified anti-EGFR antibody, or antigen-binding fragment thereof, contains a variable heavy chain that exhibits at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity to the amino acid sequence set forth in SEQ ID NO: 2 or 7; and/or a variable light chain that exhibits at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity to the amino acid sequence set forth in SEQ ID NO: 4, 9 or 11.

For example, the unmodified anti-EGFR antibody, or antigen-binding fragment, in which the glutamic acid substitution is made, contains a variable heavy chain set forth in SEQ ID NO: 2 and a variable light chain set forth in SEQ ID NO: 4. In some alternatives, the unmodified antibody, or antigen-binding fragment thereof, contains a variable heavy chain set forth in SEQ ID NO: 7 and a variable light chain set forth in SEQ ID NO: 9 or 11.

In any of the above examples, the unmodified anti-EGFR antibody provided to which the amino acid replacement is made is a humanized variant of cetuximab. In such examples, the humanized unmodified cetuximab can have a variable heavy chain set forth in SEQ ID NO: 14 and variable light chain set forth in SEQ ID NO: 15; or can have a variable heavy chain set forth in SEQ ID NO: 16 and a variable light chain set forth in SEQ ID NO: 17.

The modified anti-EGFR antibody, or antigen-binding fragment thereof, of any of the above examples can have a variable heavy chain that exhibits at least 70%, 75%, 76%, 77%, 78%, 79%, 80%, 81%, 82%, 83%, 84%, 85% sequence identity to SEQ ID NO: 2 or 7.

Any of the modified anti-EGFR antibodies, or antigen-binding fragments thereof, provided herein can be a full-length antibody, or can be an antigen-binding fragment selected from among a Fab, Fab′, F(ab′)₂, single-chain Fv (scFv), Fv, dsFv, diabody, Fd and Fd′ fragment. In some particular examples, the antigen-binding fragment is a Fab or scFv.

Included among modified anti-EGFR antibody, or antigen-binding fragment thereof, provided herein is a modified anti-EGFR antibody, or antigen-binding fragment thereof, that has a variable heavy (VH) chain with the sequence of amino acids set forth in SEQ ID NO: 74 or 75, or a sequence of amino acids that exhibits at least 85% sequence identity to SEQ ID NO: 74 or 75; and a variable light (VL) chain containing the sequence of amino acids set forth in SEQ ID NO: 4, 9 or 11, or a sequence of amino acids that exhibits at least 85%, sequence identity to SEQ ID NO: 4, 9 or 11. For example, the unmodified cetuximab antibody, or antigen-binding fragment thereof, has a variable heavy chain set forth in SEQ ID NO: 2 and a variable light chain set forth in SEQ ID NO: 4, and is modified to generate a modified anti-EGFR antibody, or antigen-binding fragment thereof, with a variable heavy (VH) chain containing the sequence of amino acids set forth in SEQ ID NO: 75, or a sequence of amino acids that exhibits at least 85%, sequence identity to SEQ ID NO: 75; and a variable light (VL) chain containing the sequence of amino acids set forth in SEQ ID NO: 4, or a sequence of amino acids that exhibits at least 85%, sequence identity to SEQ ID NO: 4. In other examples, an unmodified cetuximab antibody, or antigen-binding fragment thereof, the unmodified anti-EGFR antibody has a variable heavy chain set forth in SEQ ID NO: 7 and a variable light chain set forth in SEQ ID NO: 9, and the modified anti-EGFR antibody, or antigen-binding fragment thereof has a variable heavy (VH) chain containing the sequence of amino acids set forth in SEQ ID NO: 74, or a sequence of amino acids that exhibits at least 85%, sequence identity to SEQ ID NO: 74 and a variable light (VL) chain containing the sequence of amino acids set forth in SEQ ID NO: 9, or a sequence of amino acids that exhibits at least 85% sequence identity to SEQ ID NO: 9. In other examples, the unmodified cetuximab antibody, or antigen-binding fragment thereof, has a variable heavy chain set forth in SEQ ID NO: 7 and a variable light chain set forth in SEQ ID NO: 11 and the modified anti-EGFR antibody or antigen-binding fragment thereof has a variable heavy (VH) chain containing the sequence of amino acids set forth in SEQ ID NO: 74, or a sequence of amino acids that exhibits at least 85%, sequence identity to SEQ ID NO: 74; and a variable light (VL) chain containing the sequence of amino acids set forth in SEQ ID NO: 11, or a sequence of amino acids that exhibits at least 85% sequence identity to SEQ ID NO: 11.

In any of the examples herein, the modified anti-EGFR antibody, or antigen-binding fragment thereof, is a full-length IgG antibody that has a heavy chain variable domain set forth in either SEQ ID NO: 74 or 75 and a heavy chain constant region set forth in amino acids 120-449 of SEQ ID NO: 72, or a variant thereof that exhibits at least 85% sequence identity to amino acids 120-449 of SEQ ID NO: 72; and a light chain variable domain set forth in any of SEQ ID NOS: 4, 9 or 11 and a constant region set forth in amino acids 108-213 of SEQ ID NO: 3 or 10 or a variant thereof that exhibits at least 85% sequence identity thereto or a constant region set forth in amino acids 108-214 of SEQ ID NO: 8 or 13, or a variant thereof that exhibits at least 85% sequence identity thereto. For example, the full-length heavy chain has the sequence set forth in SEQ ID NO: 72, or a variant thereof that exhibits at least 85% sequence identity thereto, and a full-length light chain set forth in any of SEQ ID NOS: 3, 8, 10 or 13, or a variant thereof that exhibits at least 85% sequence identity thereto.

Any of the modified anti-EGFR antibodies, or antigen-binding fragments thereof, provided can contain one or more additional amino acid replacement(s) in the variable heavy chain, compared to the unmodified antibody. Non-limiting examples of additional modifications correspond to amino acid replacement(s) T023K, T023H, T023R, T023A, T023C, T023E, T023G, T023I, T023M, T023N, T023P, T023S, T023V, T023W, T023L, V024R, V024A, V024F, V024G, V024I, V024M, V024P, V024S, V024T, V024L, V024E, S025H, S025R, S025A, S025C, S025D, S025E, S025F, S025G, S025I, S025M, S025P, S025Q, S025T, S025V, S025L, G026H, G026R, G026D, G026F, G026M, G026N, G026P, G026Q, G026S, G026Y, G026L, F027H, F027R, F027A, F027D, F027E, F027G, F027M, F027P, F027Q, F027S, F027T, F027V, F027W, F027Y, F027L, S028K, S028H, S028R, S028A, S028D, S0281, S028M, S028P, S028Q, S028V, S028W, S028L, S028C, L029K, L029H, L029A, L029D, L029G, L029I, L029M, L029N, L029S, L029V, T030H, T030R, T030D, T030G, T030I, T030M, T030N, T030P, T030S, T030V, T030W, T030Y, N031K, N031H, N031D, N031E, N031G, N031I, N031T, N031V, N031L, Y032H, Y032R, Y032C, Y032M, Y032N, Y032T, Y032V, Y032L, G033E, G033M, G033S, G033T, G033Y, V034A, V034C, V034I, V034M, V034P, V034L, H035I, H035Q, W036K, W036A, W036I, W036V, W036Y, V050K, V050H, V050A, V050D, V050E, V050G, V050I, V050N, V050Q, V050T, V050L, I051K, I051H, I051A, I051C, 1051E, I051G, I051N, I051Q, I051S, I051V, I051Y, I52L, W052I, W052N, W052Y, S053H, S053R, S053A, S053C, S053G, S053I, S053M, S053P, S053Q, S053L, S053T, S053V, S053Y, G054H, G054R, G054A, G054C, G054D, G054P, G054S, G055H, G055R, G055M, G055S, G055Y, N056K, N056A, N056P, N056S, N056V, N056G, T057H, T057R, T057L, T057A, T057C, T057D, T057F, T057M, T057N, T057Q, T057W, T057Y, D058L, D058G, D058M, D058N, D058Q, Y059H, Y059R, Y059A, Y059C, Y059D, Y059E, Y059G, Y059I, Y059P, Y059Q, Y059S, Y059T, Y059V, Y059W, N060K, N060A, N060C, N060D, N060F, N060G, N060P, N060Q, N060S, N060T, N060Y, T061N, T061Q, P062G, F063H, F063R, F063L, F063A, F063C, F063D, F063G, F063M, F063N, F063Q, F063S, F063V, F063P, T064R, T064L, T064C, T064F, T064G, T064N, T064Q, T064V, S065H, S065R, S065L, S065C, S065E, S065F, S065G, S0651, S065M, S065N, S065P, S065Q, S065T, S065W, S065Y, R066L, R066A, R066C, R066E, R066F, R066N, R066P, R066Q, R066S, R066T, R066V, R066G, L067A, L067C, L067D, L067E, L067I, L067M, L067Q, L067S, L067T, L067V, L067Y, L067G, S068K, S068H, S068R, S068L, S068C, S068D, S068E, S068F, S068G, S068I, S068N, S068Q, S068T, S068V, I069A, I069C, I069G, I069Y, N070H, N070R, N070L, N070D, N070E, N070F, N070G, N070I, N070P, N070Q, N070S, N070T, N070V, N070Y, K071H, K071R, K071L, K071A, K071C, K071F, K071G, K071Q, K071S, K071T, K071V, K071W, K071Y, D072K, D072H, D072R, D072L, D072A, D072G, D072I, D072M, D072N, D072Q, D072S, D072V, D072W, D072Y, D072P, N073H, N073R, N073L, N073A, N073C, N073G, N073I, N073M, N073P, N073Q, N073S, N073T, N073V, N073W, N073Y, S074K, S074H, S074R, S074L, S074A, S074C, S074D, S074E, S074G, S074I, S074M, S074P, S074T, S074V, S074Y, K075H, K075R, K075L, K075A, K075C, K075E, K075F, K075M, K075Q, K075T, K075V, K075W, K075Y, K075G, K075P, S076H, S076R, S076L, S076A, S076C, S076D, S076E, S076F, S076M, S076P, S076Q, S076T, S076Y, S0761, S076V, Q077H, Q077R, Q077L, Q077A, Q077E, Q077G, Q077I, Q077M, Q077N, Q077S, Q077V, Q077W, Q077Y, Y093H, Y093V, Y093W, Y094R, Y094L, R097H, R097W, A098P, L099N, L099W, T100H, T100L, T100A, T100D, T100I, T100N, T100P, T100Q, T100S, T100V, T100Y, Y101H, Y101E, Y101F, Y101M, Y101W, Y102R, Y102C, Y102D, Y102I, Y102N, Y102W, D103R, D103L, D103A, D103C, D103I, D103P, D103Q, D103Y, E105H, E105T, F106L, F106V, F106W, F106Y, A107K, A107H, A107R, A107L, A107C, A107D, A107E, A107G, A107N, A107S, A107T, A107Y, Y108K, Y108H, Y108R, Y108L, Y108C, Y108F, Y108I, Y108N, Y108S, Y108T, Y108V, Y108W, W109I, W109M, W109Y, G110R, G110A, G110M, G110P, G110T, Q111K, Q111H, Q111R, Q111L, Q111D, Q111E, Q111G, Q111M, Q111P, Q111S, Q111T, Q111W, Q111Y, Q111V, Q111I, G112A, G112N, G112P, G112S, G112T and/or G112Y, with reference to the amino acid positions set forth in SEQ ID NO: 2 or 7.

In any of the examples herein, a modified anti-EGFR antibody, or antigen-binding fragment thereof, contains one or more additional amino acid replacement(s) in a variable heavy chain of the unmodified antibody corresponding to amino acid replacement(s) V24I, V24L, V24E, S25C, S25G, S25I, S25M, S25V, S25Q, S25T, S25L, S25H, S25R, S25A, S25D, F27R, S28C, L29H, T30F, N31H, N31I, N31T, N31V, Y32T, V50L, S53G, G54D, G54S, G54R, G54C, G54P, D58M, Y59E, F63R, F63C, F63G, F63M, F63V, F63P, F63S, T64N, T64V, L67G, S68F, S68Q, D72K, D72L, D72P, D72M, D72W, N73Q, S74H, S74R, S74D, S74G, S74Y, K75H, K75G, K75W, K75P, S76I, S76V, Q77R, Q77E, R97H, T100I, T100P, Y101W, Y105V, A107N, Q111I, Q111P, and/or Q111V with reference to SEQ ID NO: 2 or 7. In such examples, the corresponding amino acid positions are identified by alignment of the variable heavy chain of the antibody with the variable heavy chain set forth in SEQ ID NO: 2 or 7.

For example, the modified anti-EGFR antibody, or antigen-binding fragment thereof, can contain one or more amino acid replacement(s) in the variable heavy chain of the unmodified antibody corresponding to amino acid replacement(s) V24E, S25C, S25V, F27R, T30F, S53G, D72L, R97H, and/or Q111P. Non-limiting examples of modified anti-EGFR antibodies, and antigen-binding fragments thereof, which contain additional modifications include anti-EGFR antibody, or antigen-binding fragment thereof, with the amino acid replacements HC-Y104E/HC-Q111P; HC-S25C/HC-Y104E; HC-Y104E/LC-I29S; HC-Y104E/HC-Q111P/LC-I29S; HC-S53G/HC-Y104E; HC-S53G/HC-Y104E/HC-Q111P; HC-S25V/HC-Y104E; HC-S25V/HC-Y104E/HC-Q111P; HC-S25V/HC-S53G/HC-Y104E; HC-S25V/HC-S53G/HC-Y104E/HC-Q111P; HC-T30F/HC-Y104E; HC-T30F/HC-Y104E/HC-Q111P; HC-T30F/HC-S53G/HC-Y104E; HC-T30F/HC-S53G/HC-Y104E/HC-Q111P; HC-D72L/HC-Y104E; HC-D72L/HC-Y104E/HC-Q111P; HC-S53G/HC-D72L/HC-Y104E; or HC-S53G/HC-D72L/HC-Y104E/HC-Q111P, where HC denotes the modification in the heavy chain of the antibody or antigen binding fragment.

Included among the modified anti-EGFR antibodies and antigen fragments provided herein are those that contain a variable heavy (VH) chain having the sequence of amino acids set forth in SEQ ID NOS: 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, or 123, or a sequence of amino acids that exhibits at least 85% sequence identity to any of SEQ ID NOS: 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, or 123; and a variable light (VL) chain having the sequence of amino acids set forth in SEQ ID NO: 4, 9 or 11, or a sequence of amino acids that exhibits at least 85% sequence identity to SEQ ID NO: 4, 9 or 11.

For example, an unmodified cetuximab antibody or antigen-binding fragment thereof with a variable heavy chain set forth in SEQ ID NO: 2 and a variable light chain set forth in SEQ ID NO: 4 can be modified to generate a modified anti-EGFR antibody or antigen-binding fragment, as provided herein, that contains a variable heavy (VH) chain having the sequence of amino acids set forth in SEQ ID NO: 80, 84, 87, 90, 93, 96, 99, 102, 105, 108, 111, 114, 117, 120, or 123, or a sequence of amino acids that exhibits at least 85% sequence identity to any of SEQ ID NOS: 80, 84, 87, 90, 93, 96, 99, 102, 105, 108, 111, 114, 117, 120, or 123; and a variable light (VL) chain with the sequence of amino acids set forth in SEQ ID NO: 4, or a sequence of amino acids that exhibits at least 85% sequence identity to SEQ ID NO: 4.

In other examples, an unmodified cetuximab antibody or antigen-binding fragment thereof with a variable heavy chain set forth in SEQ ID NO: 7 and a variable light chain set forth in SEQ ID NO: 9 can be modified to generate a modified anti-EGFR antibody or antigen-binding fragment, as provided herein, that contains a variable heavy (VH) chain having the sequence of amino acids set forth in SEQ ID NO: 77, 80, 83, 86, 89, 92, 95, 98, 101, 104, 107, 110, 113, 116, 119, or 122, or a sequence of amino acids that exhibits at least 85% sequence identity to any of SEQ ID NOS: 77, 80, 83, 86, 89, 92, 95, 98, 101, 104, 107, 110, 113, 116, 119, or 122; and a variable light (VL) chain having the sequence of amino acids set forth in SEQ ID NO: 9, or a sequence of amino acids that exhibits at least 85% sequence identity to SEQ ID NO: 9.

In other examples, an unmodified cetuximab antibody or antigen-binding fragment thereof with a variable heavy chain set forth in SEQ ID NO: 7 and a variable light chain set forth in SEQ ID NO: 11 can be modified to generate a modified anti-EGFR antibody or antigen-binding fragment, as provided herein, that contains a variable heavy (VH) chain having the sequence of amino acids set forth SEQ ID NO: 77, 80, 83, 86, 89, 92, 95, 98, 101, 104, 107, 110, 113, 116, 119, or 122, or a sequence of amino acids that exhibits at least 85% sequence identity to any of SEQ ID NOS: 77, 80, 83, 86, 89, 92, 95, 98, 101, 104, 107, 110, 113, 116, 119, or 122; and a variable light (VL) chain having the sequence of amino acids set forth in SEQ ID NO: 11, or a sequence of amino acids that exhibits at least 85% sequence identity to SEQ ID NO: 11.

Other exemplary modified anti-EGFR antibodies, or antigen-binding fragments thereof, can contain a variable heavy (VH) chain having the sequence of amino acids set forth in SEQ ID NO: 74, 77 or 104, or a sequence of amino acids that exhibits at least 85% sequence identity to any of SEQ ID NOS: 74, 77 or 104; and a variable light (VL) chain having the sequence of amino acids set forth in any of SEQ ID NOS: 4, 9 or 11, or a sequence of amino acids that exhibits at least 85% sequence identity to any of SEQ ID NOS: 4, 9 or 11.

In some examples, the modified anti-EGFR antibody, or antigen-binding fragment thereof, is a full-length antibody. Such modified antibodies can have a heavy chain constant region as set forth in amino acids 120-449 of any of SEQ ID NOS: 76, 79, 82, 85, 88, 91, 94, 97, 100, 103, 106, 109, 112, 115, 118, or 121, or a variant thereof that exhibits at least 85% sequence identity to amino acids 120-449 of any of SEQ ID NOS: 76, 79, 82, 85, 88, 91, 94, 97, 100, 103, 106, 109, 112, 115, 118, or 121; and a light chain constant region as set forth in amino acids 108-213 of SEQ ID NO: 3 or 10 or a variant thereof that exhibits at least 85% sequence identity thereto or a constant region set forth in amino acids 108-214 of SEQ ID NO: 8 or 13, or a variant thereof that exhibits at least 85% sequence identity thereto. For example, such modified antibodies can have a full-length heavy chain with the sequence of amino acids set forth in any of SEQ ID NOS: 76, 79, 82, 85, 88, 91, 94, 97, 100, 103, 106, 109, 112, 115, 118, or 12, or a variant thereof that exhibits at least 85% sequence identity thereto, and a full-length light chain with the sequence of amino acids set forth in any of SEQ ID NOS: 3, 8, 10 or 13, or a variant thereof that exhibits at least 85% sequence identity thereto.

Any of the above exemplary modified anti-EGFR antibodies or antigen-binding fragments can further contain one or more amino acid replacement(s) in the variable light chain of the unmodified antibody corresponding to amino acid replacement(s) DOO1W, I002C, I002V, I002W, L003D, L003F, L003G, L0035, L003T, L003V, L003W, L003Y, L003R, L004C, L004E, L004F, L004I, L004P, L004S, L004T, L004V, L004W, L004K, L004H, L004R, T005A, T005C, T005D, T005E, T005F, T005G, T005N, T005S, T005W, T005L, T005K, T005H, T005R, T005P, R024A, R024C, R024F, R024L, R024M, R024S, R024W, R024Y, R024G, A025C, A025G, A025L, A025V, S026A, S026C, S026D, S026I, S026M, S026N, S026V, S026W, 5026L, S026G, S026H, S026R, Q027A, Q027D, Q027E, Q027F, Q027I, Q027M, Q027N, Q027P, Q027T, S028A, S028D, S028N, S028Q, S028L, S028K, 5028H, I029A, I029E, I029F, I1029S, I029T, I029R, G030A, G030E, G030F, G030I, G030M, G030P, G030Q, G030S, G030V, G030Y, G030L, G030K, G030H, G030R, T031A, T031F, T031G, T031M, T031S, T031V, T031W, T031L, T031K, T031H, N032G, I033F, I033G, I033M, I033T, I033V, I033H, I048M, I048S, I048L, I048K, K049A, K049E, K049F, K049G, K049N, K049Q, K049S, K049T, K049V, K049Y, K049L, K049H, K049R, A051T, A051L, S052A, S052C, S052D, 5052E, S052G, S052I, S052M, S052Q, S052V, S052W, S052R, S052K, E053G, S054M, I055A, I055F, S056G, S056L, S056A, S056C, S056D, S056E, S056F, S056N, S056P, S056Q, S056V, S056W, S056H, S056R, S056K, Y086F, Y086M, Y086H, Y087L, Y087C, Y087D, Y087F, Y087G, Y0871, Y087N, Y087P, Y087S, Y087T, Y087V, Y087W, Y087K, Y087H, Y087R, Q089E, N091L, N091A, N091C, N091I, N091M, N091 S, N091T, N091V, N091H, N091R, N092C, N092D, N092L, N092M, N092S, N092T, N092V, N092W, N092Y, N092H, N092K, N092R, N093T, T096L, T096C, T096M, T096V, T097L, T097A, T097D, T097G, T097Q, T097S, T097V, T097K, T097R, F098A, F098M, F098S, F098V, F098Y, G099L, G099D, G099E, G099F, G099I, G099M, G099N, G099S, G099T, G099V, G099K, G099H, Q100C, Q100D, Q100E, Q100F, Q100I, Q100M, Q100N, Q100P, Q100T, Q100V, Q100W, Q100Y, Q100K, Q100H or Q100R with reference to amino acid positions set forth in SEQ ID NO: 4, wherein corresponding amino acid position are identified by alignment of the variable light chain of the antibody with the variable light chain set forth in SEQ ID NO: 4.

In some examples, the modified anti-EGFR antibody, or antigen-binding fragment thereof, contains an amino acid replacement(s) in the variable light chain of the unmodified antibody corresponding to amino acid replacement(s) L4C, L4F, L4V, T5P, R24G, I29S, S56H and/or N91V with reference to SEQ ID NO: 4, wherein corresponding amino acid positions are identified by alignment of the variable light chain of the antibody with the variable light chain set forth in SEQ ID NO: 4. In particular examples, the modified anti-EGFR antibody, or antigen-binding fragment thereof, contains an amino acid replacement in the variable light chain of the unmodified antibody corresponding to amino acid replacement I29S with reference to SEQ ID NO: 4. Examples of such antibodies include those where the amino acid replacements are HC-Y104E/LC-I29S or HC-Y104E/HC-Q111P/LC-I29S.

Exemplary modified anti-EGFR antibodies, or antigen-binding fragments thereof, provided herein, which contain a modified variable heavy chain and a modified variable light chain, can contain a modified variable heavy (VH) chain having the sequence of amino acids set forth in any of SEQ ID NO: 74, 75, 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, or 123, or a sequence of amino acids that exhibits at least 85% sequence identity to any of SEQ ID NOS: 74, 75, 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, or 123; and a modified variable light (VL) chain having the sequence of amino acids set forth in SEQ ID NO: 125, 126, or 127, or a sequence of amino acids that exhibits at least 85% sequence identity to any of SEQ ID NOS: 125, 126 or 127.

For example, the variable heavy and light chains of an unmodified cetuximab antibody, or antigen-binding fragment thereof, as set forth in SEQ ID NOS: 2 and 4, respectively, can be modified to generate a modified anti-EGFR antibody, or antigen-binding fragment thereof, that has a modified variable heavy (VH) chain having the sequence of amino acids set forth in SEQ ID NO: 81, 84, 87, 90, 93, 96, 99, 102, 105, 108, 111, 114, 117, 120, or 123, or a sequence of amino acids that exhibits at least 85% sequence identity to any of SEQ ID NOS: 81, 84, 87, 90, 93, 96, 99, 102, 105, 108, 111, 114, 117, 120, or 123; and a modified variable light (VL) chain containing the sequence of amino acids set forth in SEQ ID NO: 126, or a sequence of amino acids that exhibits at least 85% sequence identity to SEQ ID NO: 126.

In other examples, the variable heavy and light chains of an unmodified cetuximab antibody, or antigen-binding fragment thereof, as set forth in SEQ ID NOS: 7 and 9, respectively, can be modified to generate a modified anti-EGFR antibody, or antigen-binding fragment thereof, that has a modified variable heavy (VH) chain that has the sequence of amino acids set forth in SEQ ID NO: 77, 80, 83, 86, 89, 92, 95, 98, 101, 104, 107, 110, 113, 116, 119, or 122, or a sequence of amino acids that exhibits at least 85% sequence identity to any of SEQ ID NOS: 77, 80, 83, 86, 89, 92, 95, 98, 101, 104, 107, 110, 113, 116, 119, or 122; and a modified variable light (VL) chain that has the sequence of amino acids set forth in SEQ ID NO: 125, or a sequence of amino acids that exhibits at least 85% sequence identity to SEQ ID NO: 125.

In further examples, the variable heavy and light chains of an unmodified cetuximab antibody, or antigen-binding fragment thereof, as set forth in SEQ ID NOS: 7 and 11, respectively, can be modified to generate a modified anti-EGFR antibody, or antigen-binding fragment thereof, that has a modified variable heavy (VH) chain that has the sequence of amino acids set forth in SEQ ID NO: 77, 80, 83, 86, 89, 92, 95, 98, 101, 104, 107, 110, 113, 116, 119, or 122, or a sequence of amino acids that exhibits at least 85% sequence identity to any of SEQ ID NOS: 77, 80, 83, 86, 89, 92, 95, 98, 101, 104, 107, 110, 113, 116, 119, or 122; and a modified variable light (VL) chain that has the sequence of amino acids set forth in SEQ ID NO: 127, or a sequence of amino acids that exhibits at least 85% sequence identity to SEQ ID NO: 127.

In some examples, the modified anti-EGFR antibodies, are full length IgG antibodies, that have a modified heavy chain variable region set forth in any of SEQ ID NOS: 74, 75, 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, or 123, or a sequence of amino acids that exhibits at least 85% sequence identity to any of SEQ ID NOS: 74, 75, 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, or 123 and a heavy chain constant region set forth in amino acids 120-449 of any of SEQ ID NOS: 72, 76, 79, 82, 85, 88, 91, 94, 97, 100, 103, 106, 109, 112, 115, 118, or 121, or a variant thereof that exhibits at least 85% sequence identity to amino acids 120-449 of any of SEQ ID NOS: 72, 76, 79, 82, 85, 88, 91, 94, 97, 100, 103, 106, 109, 112, 115, 118, or 121; and a modified variable light (VL) chain having the sequence of amino acids set forth in SEQ ID NO: 125, 126, or 127, or a sequence of amino acids that exhibits at least 85% sequence identity to any of SEQ ID NOS: 125, 126 or 127 and a light chain constant region set forth in amino acids 108-214 of SEQ ID NO: 124, or a variant thereof that exhibits at least 85% sequence identity to amino acids 108-214 of SEQ ID NO: 124. For example, the full-length IgG antibodies can have a full-length modified heavy chain set forth in any of SEQ ID NOS: 74, 75, 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, or 123, or a sequence of amino acids that exhibits at least 85% sequence identity to any of SEQ ID NOS: 74, 75, 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, or 123; and a full-length light chain set forth in SEQ ID NO: 124, or a variant thereof that exhibits at least 85% sequence identity to SEQ ID NO: 124.

Any of the exemplary modified anti-EGFR antibodies provided herein above can be further modified so that they are humanized. The humanized antibodies, or antigen-binding fragments, provided herein can contain a variable heavy chain that exhibits between 65% and 85% sequence identity to the variable heavy chain set forth in SEQ ID NO: 2 or 7; and a variable light chain exhibits between 65% and 85% sequence identity to the variable light chain set forth in SEQ ID NO: 4. Such humanized, modified anti-EGFR antibodies, or antigen-bind fragments thereof, can contain the amino acid replacement with glutamic acid (E) at a position corresponding to position 104 of SEQ ID NO: 2 or 7.

Exemplary humanized and modified anti-EGFR antibodies, or antigen-binding fragments thereof, provided herein have a sequence of amino acids containing the variable heavy chain set forth in SEQ ID NO: 61 or 63 or a sequence of amino acids that exhibits at least 85% sequence identity to SEQ ID NO: 61 or 63, and the variable light chain set forth in SEQ ID NO: 183, 184 or 186 or a sequence of amino acids that exhibits at least 85% sequence identity to SEQ ID NO: 183, 184 or 186.

In some examples, the unmodified anti-EGFR antibody or antigen-binding fragment thereof, having a variable heavy chain set forth in SEQ ID NO: 2 and a variable light chain set forth in SEQ ID NO: 4, is humanized and modified to generate an anti-EGFR antibody or antigen-binding fragment that has a variable heavy chain set forth in SEQ ID NO: 63 or a sequence of amino acids that exhibits at least 85% sequence identity to SEQ ID NO: 63, and a variable light chain set forth in SEQ ID NO: 184 or a sequence of amino acids that exhibits at least 85% sequence identity to SEQ ID NO: 184.

In other examples, the unmodified anti-EGFR antibody or antigen-binding fragment thereof, having a variable heavy chain set forth in SEQ ID NO: 7 and a variable light chain set forth in SEQ ID NO: 9, is humanized and modified to generate an anti-EGFR antibody or antigen-binding fragment that has a variable heavy chain set forth in SEQ ID NO: 61 or a sequence of amino acids that exhibits at least 85% sequence identity to SEQ ID NO: 61, and a variable light chain set forth in SEQ ID NO: 183 or a sequence of amino acids that exhibits at least 85% sequence identity to SEQ ID NO: 183.

In further examples, the unmodified anti-EGFR antibody or antigen-binding fragment thereof, having a variable heavy chain set forth in SEQ ID NO: 7 and a variable light chain set forth in SEQ ID NO: 11, is humanized and modified to generate an anti-EGFR antibody or antigen-binding fragment that has a variable heavy chain set forth in SEQ ID NO: 61 or a sequence of amino acids that exhibits at least 85% sequence identity to SEQ ID NO: 61, and a variable light chain set forth in SEQ ID NO: 186 or a sequence of amino acids that exhibits at least 85% sequence identity to SEQ ID NO: 186.

In some examples, the humanized, modified anti-EGFR antibody, or antigen-binding fragment thereof, is a full-length IgG antibody, which has a heavy chain having the sequence of amino acids set forth in SEQ ID NO: 59 or a sequence of amino acids that exhibits at least 85% sequence identity to SEQ ID NO: 59, and a light chain with a sequence of amino acids set forth in SEQ ID NO: 181 or a sequence of amino acids that exhibits at least 85% sequence identity to SEQ ID NO: 181.

Any of the humanized, modified anti-EGFR antibodies, or antigen-binding fragments thereof, described herein above can contain additional modifications, such as one or more amino acid replacement(s) in the variable heavy chain corresponding to amino acid replacement(s) selected from among T023K, T023H, T023R, T023A, T023C, T023E, T023G, T023I, T023M, T023N, T023P, T023S, T023V, T023W, T023L, V024R, V024A, V024F, V024G, V024I, V024M, V024P, V024S, V024T, V024L, V024E, S025H, S025R, S025A, S025C, S025D, S025E, S025F, S025G, S025I, S025M, S025P, S025Q, S025T, S025V, S025L, G026H, G026R, G026D, G026F, G026M, G026N, G026P, G026Q, G026S, G026Y, G026L, F027H, F027R, F027A, F027D, F027E, F027G, F027M, F027P, F027Q, F027S, F027T, F027V, F027W, F027Y, F027L, S028K, S028H, S028R, S028A, S028D, S0281, S028M, S028P, S028Q, S028V, S028W, S028L, S028C, L029K, L029H, L029A, L029D, L029G, L029I, L029M, L029N, L029S, L029V, T030H, T030R, T030D, T030G, T030I, T030M, T030N, T030P, T030S, T030V, T030W, T030Y, N031K, N031H, N031D, N031E, N031G, N031I, N031T, N031V, N031L, Y032H, Y032R, Y032C, Y032M, Y032N, Y032T, Y032V, Y032L, G033E, G033M, G033S, G033T, G033Y, V034A, V034C, V034I, V034M, V034P, V034L, H035I, H035Q, W036K, W036A, W036I, W036V, W036Y, V050K, V050H, V050A, V050D, V050E, V050G, V050I, V050N, V050Q, V050T, V050L, I051K, I051H, I051A, I051C, 1051E, I051G, I051N, I051Q, I051S, I051V, I051Y, I051L, W052I, W052N, W052Y, S053H, S053R, S053A, S053C, S053G, S053I, S053M, S053P, S053Q, S053L, S053T, S053V, S053Y, G054H, G054R, G054A, G054C, G054D, G054P, G054S, G055H, G055R, G055M, G055S, G055Y, N056K, N056A, N056P, N056S, N056V, N056G, T057H, T057R, T057L, T057A, T057C, T057D, T057F, T057M, T057N, T057Q, T057W, T057Y, D058L, D058G, D058M, D058N, D058Q, Y059H, Y059R, Y059A, Y059C, Y059D, Y059E, Y059G, Y059I, Y059P, Y059Q, Y059S, Y059T, Y059V, Y059W, N060K, N060A, N060C, N060D, N060F, N060G, N060P, N060Q, N060S, N060T, N060Y, T061N, T061Q, P062G, F063H, F063R, F063L, F063A, F063C, F063D, F063G, F063M, F063N, F063Q, F063S, F063V, F063P, T064R, T064L, T064C, T064F, T064G, T064N, T064Q, T064V, S065H, S065R, S065L, S065C, S065E, S065F, S065G, S0651, S065M, S065N, S065P, S065Q, S065T, S065W, S065Y, R066L, R066A, R066C, R066E, R066F, R066N, R066P, R066Q, R066S, R066T, R066V, R066G, L067A, L067C, L067D, L067E, L067I, L067M, L067Q, L067S, L067T, L067V, L067Y, L067G, S068K, S068H, S068R, S068L, S068C, S068D, S068E, S068F, S068G, S068I, S068N, S068Q, S068T, S068V, I069A, I069C, I069G, I069Y, N070H, N070R, N070L, N070D, N070E, N070F, N070G, N070I, N070P, N070Q, N070S, N070T, N070V, N070Y, K071H, K071R, K071L, K071A, K071C, K071F, K071G, K071Q, K071S, K071T, K071V, K071W, K071Y, D072K, D072H, D072R, D072L, D072A, D072G, D072I, D072M, D072N, D072Q, D072S, D072V, D072W, D072Y, D072P, N073H, N073R, N073L, N073A, N073C, N073G, N073I, N073M, N073P, N073Q, N073S, N073T, N073V, N073W, N073Y, S074K, S074H, S074R, S074L, S074A, S074C, S074D, S074E, S074G, S074I, S074M, S074P, S074T, S074V, S074Y, K075H, K075R, K075L, K075A, K075C, K075E, K075F, K075M, K075Q, K075T, K075V, K075W, K075Y, K075G, K075P, S076H, S076R, S076L, S076A, S076C, S076D, S076E, S076F, S076M, S076P, S076Q, S076T, S076Y, S0761, S076V, Q077H, Q077R, Q077L, Q077A, Q077E, Q077G, Q077I, Q077M, Q077N, Q077S, Q077V, Q077W, Q077Y, Y093H, Y093V, Y093W, Y094R, Y094L, R097H, R097W, A098P, L099N, L099W, T100H, T100L, T100A, T100D, T100I, T100N, T100P, T100Q, T100S, T100V, T100Y, Y101H, Y101E, Y101F, Y101M, Y101W, Y102R, Y102C, Y102D, Y102I, Y102N, Y102W, D103R, D103L, D103A, D103C, D103I, D103P, D103Q, D103Y, E105H, E105T, F106L, F106V, F106W, F106Y, A107K, A107H, A107R, A107L, A107C, A107D, A107E, A107G, A107N, A107S, A107T, A107Y, Y108K, Y108H, Y108R, Y108L, Y108C, Y108F, Y108I, Y108N, Y108S, Y108T, Y108V, Y108W, W109I, W109M, W109Y, G110R, G110A, G110M, G110P, G110T, Q111K, Q111H, Q111R, Q111L, Q111D, Q111E, Q111G, Q111M, Q111P, Q111S, Q111T, Q111W, Q111Y, Q111V, Q111I, G112A, G112N, G112P, G112S, G112T and G112Y, with reference to positions of the unmodified variable heavy chain set forth in SEQ ID NO: 2 or 7; and/or

one or more amino acid replacement(s) in a variable light chain of the unmodified antibody corresponding to amino acid replacement(s) D001 W, I002C, I002V, I002W, L003D, L003F, L003G, L0035, L003T, L003V, L003W, L003Y, L003R, L004C, L004E, L004F, L004I, L004P, L004S, L004T, L004V, L004W, L004K, L004H, L004R, T005A, T005C, T005D, T005E, T005F, T005G, T005N, T005S, T005W, T005L, T005K, T005H, T005R, T005P, R024A, R024C, R024F, R024L, R024M, R024S, R024W, R024Y, R024G, A025C, A025G, A025L, A025V, S026A, S026C, S026D, S026I, S026M, S026N, S026V, S026W, S026L, S026G, S026H, S026R, Q027A, Q027D, Q027E, Q027F, Q027I, Q027M, Q027N, Q027P, Q027T, S028A, S028D, S028N, S028Q, S028L, S028K, S028H, I029A, I029E, I029F, I029S, I029T, I029R, G030A, G030E, G030F, G030I, G030M, G030P, G030Q, G030S, G030V, G030Y, G030L, G030K, G030H, G030R, T031A, T031F, T031G, T031M, T031S, T031V, T031W, T031L, T031K, T031H, N032G, I033F, I033G, I033M, I033T, I033V, I033H, I048M, I048S, I048L, I048K, K049A, K049E, K049F, K049G, K049N, K049Q, K049S, K049T, K049V, K049Y, K049L, K049H, K049R, A051T, A051L, S052A, S052C, S052D, S052E, S052G, S052I, S052M, S052Q, S052V, S052W, S052R, S052K, E053G, S054M, I055A, I055F, S056G, S056L, S056A, S056C, S056D, S056E, S056F, S056N, S056P, S056Q, S056V, S056W, S056H, S056R, S056K, Y086F, Y086M, Y086H, Y087L, Y087C, Y087D, Y087F, Y087G, Y0871, Y087N, Y087P, Y087S, Y087T, Y087V, Y087W, Y087K, Y087H, Y087R, Q089E, N091L, N091A, N091C, N091I, N091M, N091S, N091 T, N091V, N091H, N091R, N092C, N092D, N092L, N092M, N092S, N092T, N092V, N092W, N092Y, N092H, N092K, N092R, N093T, T096L, T096C, T096M, T096V, T097L, T097A, T097D, T097G, T097Q, T097S, T097V, T097K, T097R, F098A, F098M, F098S, F098V, F098Y, G099L, G099D, G099E, G099F, G099I, G099M, G099N, G099S, G099T, G099V, G099K, G099H, Q100C, Q100D, Q100E, Q100F, Q100I, Q100M, Q100N, Q100P, Q100T, Q100V, Q100W, Q100Y, Q100K, Q100H or Q100R with reference to amino acid positions set forth in SEQ ID NO: 4, wherein corresponding amino acid positions are identified by alignment of the variable light chain of the antibody with the variable light chain set forth in SEQ ID NO: 4.

Any of the modified anti-EGFR antibodies, or antigen-binding fragments thereof, can further contain an amino acid replacement(s) in a variable heavy chain of the unmodified antibody corresponding to amino acid replacement V24I, V24L, V24E, S25C, S25G, S25I, S25M, S25V, S25Q, S25T, S25L, S25H, S25R, S25A, S25D, F27R, S28C, L29H, T30F, N31H, N31I, N31T, N31V, Y32T, V50L, S53G, G54D, G54S, G54R, G54C, G54P, D58M, Y59E, F63R, F63C, F63G, F63M, F63V, F63P, F63S, T64N, T64V, L67G, S68F, S68Q, D72K, D72L, D72P, D72M, D72W, N73Q, S74H, S74R, S74D, S74G, S74Y, K75H, K75G, K75W, K75P, S76I, S76V, Q77R, Q77E, R97H, T100I, T100P, Y101W, Y105V, A107N, Q111I, Q111P, Q111V with reference to SEQ ID NO: 2 or 7, wherein corresponding amino acid positions are identified by alignment of the variable heavy chain of the antibody with the variable heavy chain set forth in SEQ ID NO: 2 or 7.

In any of such examples the humanized, modified anti-EGFR antibodies, or antigen-binding fragments thereof, provided herein, contain one or more further amino acid replacement(s) in the variable heavy chain corresponding to amino acid replacement (s) V24E, S25C, S25V, F27R, T30F, S53G, D72L, R97H, and Q111P of the unmodified antibody. For example, a humanized, modified anti-EGFR antibody or antigen fragment described herein can contain the amino acid replacements in the variable heavy chain or full-length heavy chain corresponding to HC-Y104E/HC-Q111P; HC-S25C/HC-Y104E; HC-Y104E/LC-1295; HC-Y104E/HC-Q111P/LC-I29S; HC-S53G/HC-Y104E; HC-S53G/HC-Y104E/HC-Q111P; HC-S25V/HC-Y104E; HC-S25V/HC-Y104E/HC-Q111P; HC-S25V/HC-S53G/HC-Y104E; HC-S25V/HC-S53G/HC-Y104E/HC-Q111P; HC-T30F/HC-Y104E; HC-T30F/HC-Y104E/HC-Q111P; HC-T30F/HC-S53G/HC-Y104E; HC-T30F/HC-S53G/HC-Y104E/HC-Q111P; HC-D72L/HC-Y104E; HC-D72L/HC-Y104E/HC-Q111P; HC-S53G/HC-D72L/HC-Y104E; or HC-S53G/HC-D72L/HC-Y104E/HC-Q111P. In particular examples, the humanized, modified anti-EGFR antibody, or antigen-binding fragment thereof, contains the amino acid replacements HC-Y104E/HC-Q111P or HC-T30F/HC-Y104E/HC-Q111P.

For example, among non-limiting examples of a humanized, modified anti-EGFR antibody, or antigen-binding fragment thereof, provided herein is an antibody that contains:

a) the variable heavy chain set forth in SEQ ID NO: 131 or 133 or a sequence of amino acids that exhibits at least 85% sequence identity to SEQ ID NO: 131 or 133, and the variable light chain set forth in SEQ ID NO: 155, 156 or 158 or a sequence of amino acids that exhibits at least 85% sequence identity to SEQ ID NO: 155, 156 or 158;

b) the variable heavy chain set forth in SEQ ID NO: 131 or 133 or a sequence of amino acids that exhibits at least 85% sequence identity to SEQ ID NO: 131 or 133, and the variable light chain set forth in SEQ ID NO: 162, 163 or 165 or a sequence of amino acids that exhibits at least 85% sequence identity to SEQ ID NO: 162, 163 or 165;

c) the variable heavy chain set forth in SEQ ID NO: 137 or 139 or a sequence of amino acids that exhibits at least 85% sequence identity to SEQ ID NO: 137 or 139, and the variable light chain set forth in SEQ ID NO: 155, 156 or 158 or a sequence of amino acids that exhibits at least 85% sequence identity to SEQ ID NO: 155, 156 or 158;

d) the variable heavy chain set forth in SEQ ID NO: 131 or 133 or a sequence of amino acids that exhibits at least 85% sequence identity to SEQ ID NO: 131 or 133, and the variable light chain set forth in SEQ ID NO: 169, 170 or 172 or a sequence of amino acids that exhibits at least 85% sequence identity to SEQ ID NO: 169, 170 or 172;

e) the variable heavy chain set forth in SEQ ID NO: 131 or 133 or a sequence of amino acids that exhibits at least 85% sequence identity to SEQ ID NO: 131 or 133, and the variable light chain set forth in SEQ ID NO: 176, 177 or 179 or a sequence of amino acids that exhibits at least 85% sequence identity to SEQ ID NO: 176, 177 or 179;

f) the variable heavy chain set forth in SEQ ID NO: 131 or 133 or a sequence of amino acids that exhibits at least 85% sequence identity to SEQ ID NO: 131 or 133 and the variable light chain set forth in SEQ ID NO: 183, 184 or 186 or a sequence of amino acids that exhibits at least 85% sequence identity to SEQ ID NO: 183, 184 or 186;

g) the variable heavy chain set forth in SEQ ID NO: 137 or 139 or a sequence of amino acids that exhibits at least 85% sequence identity to SEQ ID NO: 137 or 139, and the variable light chain set forth in SEQ ID NO: 183, 184 or 186 or a sequence of amino acids that exhibits at least 85% sequence identity to SEQ ID NO: 183, 184 or 186;

h) the variable heavy chain set forth in SEQ ID NO: 131 or 133 or a sequence of amino acids that exhibits at least 85% sequence identity to SEQ ID NO: 131 or 133, and the variable light chain set forth in SEQ ID NO: 190, 191 or 193 or a sequence of amino acids that exhibits at least 85% sequence identity to SEQ ID NO: 190, 191 or 193;

i) the variable heavy chain set forth in SEQ ID NO: 143 or 145 or a sequence of amino acids that exhibits at least 85% sequence identity to SEQ ID NO: 143 or 145, and the variable light chain set forth in SEQ ID NO: 183, 184 or 186 or a sequence of amino acids that exhibits at least 85% sequence identity to SEQ ID NO: 183, 184 or 186;

j) the variable heavy chain set forth in SEQ ID NO: 149 or 151 or a sequence of amino acids that exhibits at least 85% sequence identity to SEQ ID NO: 149 or 151, and the variable light chain set forth in SEQ ID NO: 197, 198 or 200 or a sequence of amino acids that exhibits at least 85% sequence identity to SEQ ID NO: 197, 198 or 200;

k) the variable heavy chain set forth in SEQ ID NO: 143 or 145 or a sequence of amino acids that exhibits at least 85% sequence identity to SEQ ID NO: 143 or 145, and the variable light chain set forth in SEQ ID NO: 197, 198 or 200 or a sequence of amino acids that exhibits at least 85% sequence identity to SEQ ID NO: 197, 198 or 200;

l) the variable heavy chain set forth in SEQ ID NO: 149 or 151 or a sequence of amino acids that exhibits at least 85% sequence identity to SEQ ID NO: 149 or 151, and the variable light chain set forth in SEQ ID NO: 204, 205 or 207 or a sequence of amino acids that exhibits at least 85% sequence identity to SEQ ID NO: 204, 205 or 207;

m) the variable heavy chain set forth in SEQ ID NO: 143 or 145 or a sequence of amino acids that exhibits at least 85% sequence identity to SEQ ID NO: 143 or 145, and the variable light chain set forth in SEQ ID NO: 204, 205 or 207 or a sequence of amino acids that exhibits at least 85% sequence identity to SEQ ID NO: 204, 205 or 207;

n) the variable heavy chain set forth in SEQ ID NO: 211 or 213 or a sequence of amino acids that exhibits at least 85% sequence identity to SEQ ID NO: 211 or 213, and the variable light chain set forth in SEQ ID NO: 253, 254 or 256 or a sequence of amino acids that exhibits at least 85% sequence identity to SEQ ID NO: 253, 254 or 256;

o) the variable heavy chain set forth in SEQ ID NO: 217 or 219 or a sequence of amino acids that exhibits at least 85% sequence identity to SEQ ID NO: 217 or 219, and the variable light chain set forth in SEQ ID NO: 253, 254 or 256 or a sequence of amino acids that exhibits at least 85% sequence identity to SEQ ID NO: 253, 254 or 256;

p) the variable heavy chain set forth in SEQ ID NO: 223 or 225 or a sequence of amino acids that exhibits at least 85% sequence identity to SEQ ID NO: 223 or 225, and the variable light chain set forth in SEQ ID NO: 260, 261 or 263 or a sequence of amino acids that exhibits at least 85% sequence identity to SEQ ID NO: 260, 261 or 263;

q) the variable heavy chain set forth in SEQ ID NO: 229 or 231 or a sequence of amino acids that exhibits at least 85% sequence identity to SEQ ID NO: 229 or 231, and the variable light chain set forth in SEQ ID NO: 260, 261 or 263 or a sequence of amino acids that exhibits at least 85% sequence identity to SEQ ID NO: 260, 261 or 263;

r) the variable heavy chain set forth in SEQ ID NO: 235 or 237 or a sequence of amino acids that exhibits at least 85% sequence identity to SEQ ID NO: 235 or 237, and the variable light chain set forth in SEQ ID NO: 267, 268 or 270 or a sequence of amino acids that exhibits at least 85% sequence identity to SEQ ID NO: 267, 268 or 270;

s) the variable heavy chain set forth in SEQ ID NO: 241 or 243 or a sequence of amino acids that exhibits at least 85% sequence identity to SEQ ID NO: 241 or 243, and the variable light chain set forth in SEQ ID NO: 274, 275 or 277 or a sequence of amino acids that exhibits at least 85% sequence identity to SEQ ID NO: 274, 275 or 277;

t) the variable heavy chain set forth in SEQ ID NO: 223 or 225 or a sequence of amino acids that exhibits at least 85% sequence identity to SEQ ID NO: 223 or 225, and the variable light chain set forth in SEQ ID NO: 274, 275 or 277 or a sequence of amino acids that exhibits at least 85% sequence identity to SEQ ID NO: 274, 275 or 277;

u) the variable heavy chain set forth in SEQ ID NO: 229 or 231 or a sequence of amino acids that exhibits at least 85% sequence identity to SEQ ID NO: 229 or 231, and the variable light chain set forth in SEQ ID NO: 274, 275 or 277 or a sequence of amino acids that exhibits at least 85% sequence identity to SEQ ID NO: 274, 275 or 277;

v) the variable heavy chain set forth in SEQ ID NO: 235 or 237 or a sequence of amino acids that exhibits at least 85% sequence identity to SEQ ID NO: 235 or 237, and the variable light chain set forth in SEQ ID NO: 281, 282 or 284 or a sequence of amino acids that exhibits at least 85% sequence identity to SEQ ID NO: 281, 282 or 284;

w) the variable heavy chain set forth in SEQ ID NO: 247 or 249 or a sequence of amino acids that exhibits at least 85% sequence identity to SEQ ID NO: 247 or 249, and the variable light chain set forth in SEQ ID NO: 281, 282 or 284 or a sequence of amino acids that exhibits at least 85% sequence identity to SEQ ID NO: 281, 282 or 284;

x) the variable heavy chain set forth in SEQ ID NO: 223 or 225 or a sequence of amino acids that exhibits at least 85% sequence identity to SEQ ID NO: 223 or 225, and the variable light chain set forth in SEQ ID NO: 281, 282 or 284 or a sequence of amino acids that exhibits at least 85% sequence identity to SEQ ID NO: 281, 282 or 284;

y) the variable heavy chain set forth in SEQ ID NO: 229 or 231 or a sequence of amino acids that exhibits at least 85% sequence identity to SEQ ID NO: 229 or 231, and the variable light chain set forth in SEQ ID NO: 281, 282 or 284 or a sequence of amino acids that exhibits at least 85% sequence identity to SEQ ID NO: 281, 282 or 284;

z) the variable heavy chain set forth in SEQ ID NO: 235 or 237 or a sequence of amino acids that exhibits at least 85% sequence identity to SEQ ID NO: 235 or 237, and the variable light chain set forth in SEQ ID NO: 288, 289 or 291 or a sequence of amino acids that exhibits at least 85% sequence identity to SEQ ID NO: 288, 289 or 291;

aa) the variable heavy chain set forth in SEQ ID NO: 247 or 249 or a sequence of amino acids that exhibits at least 85% sequence identity to SEQ ID NO: 247 or 249, and the variable light chain set forth in SEQ ID NO: 288, 289 or 291 or a sequence of amino acids that exhibits at least 85% sequence identity to SEQ ID NO: 288, 289 or 291;

bb) the variable heavy chain set forth in SEQ ID NO: 223 or 225 or a sequence of amino acids that exhibits at least 85% sequence identity to SEQ ID NO: 223 or 225, and the variable light chain set forth in SEQ ID NO: 288, 289 or 291 or a sequence of amino acids that exhibits at least 85% sequence identity to SEQ ID NO: 288, 289 or 291;

cc) the variable heavy chain set forth in SEQ ID NO: 229 or 231 or a sequence of amino acids that exhibits at least 85% sequence identity to SEQ ID NO: 229 or 231, and the variable light chain set forth in SEQ ID NO: 288, 289 or 291 or a sequence of amino acids that exhibits at least 85% sequence identity to SEQ ID NO: 288, 289 or 291;

dd) the variable heavy chain set forth in SEQ ID NO: 235 or 237 or a sequence of amino acids that exhibits at least 85% sequence identity to SEQ ID NO: 235 or 237, and the variable light chain set forth in SEQ ID NO: 295, 296 or 298 or a sequence of amino acids that exhibits at least 85% sequence identity to SEQ ID NO: 295, 296 or 298;

ee) the variable heavy chain set forth in SEQ ID NO: 247 or 249 or a sequence of amino acids that exhibits at least 85% sequence identity to SEQ ID NO: 247 or 249, and the variable light chain set forth in SEQ ID NO: 302, 303 or 305 or a sequence of amino acids that exhibits at least 85% sequence identity to SEQ ID NO: 302, 303 or 305;

ff) the variable heavy chain set forth in SEQ ID NO: 211 or 213 or a sequence of amino acids that exhibits at least 85% sequence identity to SEQ ID NO: 211 or 213, and the variable light chain set forth in SEQ ID NO: 302, 303 or 305 or a sequence of amino acids that exhibits at least 85% sequence identity to SEQ ID NO: 302, 303 or 305;

gg) the variable heavy chain set forth in SEQ ID NO: 211 or 213 or a sequence of amino acids that exhibits at least 85% sequence identity to SEQ ID NO: 211 or 213, and the variable light chain set forth in SEQ ID NO: 281, 282 or 284 or a sequence of amino acids that exhibits at least 85% sequence identity to SEQ ID NO: 281, 282 or 284;

hh) the variable heavy chain set forth in SEQ ID NO: 211 or 213 or a sequence of amino acids that exhibits at least 85% sequence identity to SEQ ID NO: 211 or 213, and the variable light chain set forth in SEQ ID NO: 288, 289 or 291 or a sequence of amino acids that exhibits at least 85% sequence identity to SEQ ID NO: 288, 289 or 291.

In some examples, an unmodified anti-EGFR antibody or antigen-binding fragment thereof, that has a variable heavy chain set forth in SEQ ID NO: 2 and a variable light chain set forth in SEQ ID NO: 4, is humanized and modified to generate a modified anti-EGFR antibody, or antigen-binding fragment thereof, that contains:

a) the variable heavy chain set forth in SEQ ID NO: 133 or a sequence of amino acids that exhibits at least 85% sequence identity to SEQ ID NO: 133, and the variable light chain set forth in SEQ ID NO: 156 or a sequence of amino acids that exhibits at least 85% sequence identity to SEQ ID NO: 156;

b) the variable heavy chain set forth in SEQ ID NO: 133 or a sequence of amino acids that exhibits at least 85% sequence identity to SEQ ID NO: 133, and the variable light chain set forth in SEQ ID NO: 163 or a sequence of amino acids that exhibits at least 85% sequence identity to SEQ ID NO: 163;

c) the variable heavy chain set forth in SEQ ID NO: 139 or a sequence of amino acids that exhibits at least 85% sequence identity to SEQ ID NO: 139, and the variable light chain set forth in SEQ ID NO: 156 or a sequence of amino acids that exhibits at least 85% sequence identity to SEQ ID NO: 156;

d) the variable heavy chain set forth in SEQ ID NO: 133 or a sequence of amino acids that exhibits at least 85% sequence identity to SEQ ID NO: 133, and the variable light chain set forth in SEQ ID NO: 170 or a sequence of amino acids that exhibits at least 85% sequence identity to SEQ ID NO: 170;

e) the variable heavy chain set forth in SEQ ID NO: 133 or a sequence of amino acids that exhibits at least 85% sequence identity to SEQ ID NO: 133, and the variable light chain set forth in SEQ ID NO: 177 or a sequence of amino acids that exhibits at least 85% sequence identity to SEQ ID NO: 177;

f) the variable heavy chain set forth in SEQ ID NO: 133 or a sequence of amino acids that exhibits at least 85% sequence identity to SEQ ID NO: 133 and the variable light chain set forth in SEQ ID NO: 184 or a sequence of amino acids that exhibits at least 85% sequence identity to SEQ ID NO: 184;

g) the variable heavy chain set forth in SEQ ID NO: 139 or a sequence of amino acids that exhibits at least 85% sequence identity to SEQ ID NO: 139, and the variable light chain set forth in SEQ ID NO: 184 or a sequence of amino acids that exhibits at least 85% sequence identity to SEQ ID NO: 184;

h) the variable heavy chain set forth in SEQ ID NO: 133 or a sequence of amino acids that exhibits at least 85% sequence identity to SEQ ID NO: 133, and the variable light chain set forth in SEQ ID NO: 191 or a sequence of amino acids that exhibits at least 85% sequence identity to SEQ ID NO: 191;

i) the variable heavy chain set forth in SEQ ID NO: 145 or a sequence of amino acids that exhibits at least 85% sequence identity to SEQ ID NO: 145, and the variable light chain set forth in SEQ ID NO: 184 or a sequence of amino acids that exhibits at least 85% sequence identity to SEQ ID NO: 184;

j) the variable heavy chain set forth in SEQ ID NO: 151 or a sequence of amino acids that exhibits at least 85% sequence identity to SEQ ID NO: 151, and the variable light chain set forth in SEQ ID NO: 198 or a sequence of amino acids that exhibits at least 85% sequence identity to SEQ ID NO: 198;

k) the variable heavy chain set forth in SEQ ID NO: 145 or a sequence of amino acids that exhibits at least 85% sequence identity to SEQ ID NO: 145, and the variable light chain set forth in SEQ ID NO: 198 or a sequence of amino acids that exhibits at least 85% sequence identity to SEQ ID NO: 198;

l) the variable heavy chain set forth in SEQ ID NO: 151 or a sequence of amino acids that exhibits at least 85% sequence identity to SEQ ID NO: 151, and the variable light chain set forth in SEQ ID NO: 205 or a sequence of amino acids that exhibits at least 85% sequence identity to SEQ ID NO: 205;

m) the variable heavy chain set forth in SEQ ID NO: 145 or a sequence of amino acids that exhibits at least 85% sequence identity to SEQ ID NO: 145, and the variable light chain set forth in SEQ ID NO: 205 or a sequence of amino acids that exhibits at least 85% sequence identity to SEQ ID NO: 205;

n) the variable heavy chain set forth in SEQ ID NO: 213 or a sequence of amino acids that exhibits at least 85% sequence identity to SEQ ID NO: 213, and the variable light chain set forth in SEQ ID NO: 254 or a sequence of amino acids that exhibits at least 85% sequence identity to SEQ ID NO: 254;

o) the variable heavy chain set forth in SEQ ID NO: 219 or a sequence of amino acids that exhibits at least 85% sequence identity to SEQ ID NO: 219, and the variable light chain set forth in SEQ ID NO: 254 or a sequence of amino acids that exhibits at least 85% sequence identity to SEQ ID NO: 254;

p) the variable heavy chain set forth in SEQ ID NO: 225 or a sequence of amino acids that exhibits at least 85% sequence identity to SEQ ID NO: 225, and the variable light chain set forth in SEQ ID NO: 261 or a sequence of amino acids that exhibits at least 85% sequence identity to SEQ ID NO: 261;

q) the variable heavy chain set forth in SEQ ID NO: 231 or a sequence of amino acids that exhibits at least 85% sequence identity to SEQ ID NO: 231, and the variable light chain set forth in SEQ ID NO: 261 or a sequence of amino acids that exhibits at least 85% sequence identity to SEQ ID NO: 261;

r) the variable heavy chain set forth in SEQ ID NO: 237 or a sequence of amino acids that exhibits at least 85% sequence identity to SEQ ID NO: 237, and the variable light chain set forth in SEQ ID NO: 268 or a sequence of amino acids that exhibits at least 85% sequence identity to SEQ ID NO: 268;

s) the variable heavy chain set forth in SEQ ID NO: 243 or a sequence of amino acids that exhibits at least 85% sequence identity to SEQ ID NO: 243, and the variable light chain set forth in SEQ ID NO: 275 or a sequence of amino acids that exhibits at least 85% sequence identity to SEQ ID NO: 275;

t) the variable heavy chain set forth in SEQ ID NO: 225 or a sequence of amino acids that exhibits at least 85% sequence identity to SEQ ID NO: 225, and the variable light chain set forth in SEQ ID NO: 275 or a sequence of amino acids that exhibits at least 85% sequence identity to SEQ ID NO: 275;

u) the variable heavy chain set forth in SEQ ID NO: 231 or a sequence of amino acids that exhibits at least 85% sequence identity to SEQ ID NO: 231, and the variable light chain set forth in SEQ ID NO: 275 or a sequence of amino acids that exhibits at least 85% sequence identity to SEQ ID NO: 275;

v) the variable heavy chain set forth in SEQ ID NO: 237 or a sequence of amino acids that exhibits at least 85% sequence identity to SEQ ID NO: 237, and the variable light chain set forth in SEQ ID NO: 282 or a sequence of amino acids that exhibits at least 85% sequence identity to SEQ ID NO: 282;

w) the variable heavy chain set forth in SEQ ID NO: 249 or a sequence of amino acids that exhibits at least 85% sequence identity to SEQ ID NO: 249, and the variable light chain set forth in SEQ ID NO: 282 or a sequence of amino acids that exhibits at least 85% sequence identity to SEQ ID NO: 282;

x) the variable heavy chain set forth in SEQ ID NO: 225 or a sequence of amino acids that exhibits at least 85% sequence identity to SEQ ID NO: 225, and the variable light chain set forth in SEQ ID NO: 282 or a sequence of amino acids that exhibits at least 85% sequence identity to SEQ ID NO: 282;

y) the variable heavy chain set forth in SEQ ID NO: 231 or a sequence of amino acids that exhibits at least 85% sequence identity to SEQ ID NO: 231, and the variable light chain set forth in SEQ ID NO: 282 or a sequence of amino acids that exhibits at least 85% sequence identity to SEQ ID NO: 282;

z) the variable heavy chain set forth in SEQ ID NO: 237 or a sequence of amino acids that exhibits at least 85% sequence identity to SEQ ID NO: 237, and the variable light chain set forth in SEQ ID NO: 289 or a sequence of amino acids that exhibits at least 85% sequence identity to SEQ ID NO: 289;

aa) the variable heavy chain set forth in SEQ ID NO: 249 or a sequence of amino acids that exhibits at least 85% sequence identity to SEQ ID NO: 249, and the variable light chain set forth in SEQ ID NO: 289 or a sequence of amino acids that exhibits at least 85% sequence identity to SEQ ID NO: 289;

bb) the variable heavy chain set forth in SEQ ID NO: 225 or a sequence of amino acids that exhibits at least 85% sequence identity to SEQ ID NO: 225, and the variable light chain set forth in SEQ ID NO: 289 or a sequence of amino acids that exhibits at least 85% sequence identity to SEQ ID NO: 289;

cc) the variable heavy chain set forth in SEQ ID NO: 231 or a sequence of amino acids that exhibits at least 85% sequence identity to SEQ ID NO: 231, and the variable light chain set forth in SEQ ID NO: 289 or a sequence of amino acids that exhibits at least 85% sequence identity to SEQ ID NO: 289;

dd) the variable heavy chain set forth in SEQ ID NO: 237 or a sequence of amino acids that exhibits at least 85% sequence identity to SEQ ID NO: 237, and the variable light chain set forth in SEQ ID NO: 296 or a sequence of amino acids that exhibits at least 85% sequence identity to SEQ ID NO: 296;

ee) the variable heavy chain set forth in SEQ ID NO: 249 or a sequence of amino acids that exhibits at least 85% sequence identity to SEQ ID NO: 249, and the variable light chain set forth in SEQ ID NO: 303 or a sequence of amino acids that exhibits at least 85% sequence identity to SEQ ID NO: 303;

ff) the variable heavy chain set forth in SEQ ID NO: 213 or a sequence of amino acids that exhibits at least 85% sequence identity to SEQ ID NO: 213, and the variable light chain set forth in SEQ ID NO: 303 or a sequence of amino acids that exhibits at least 85% sequence identity to SEQ ID NO: 303;

gg) the variable heavy chain set forth in SEQ ID NO: 213 or a sequence of amino acids that exhibits at least 85% sequence identity to SEQ ID NO: 213, and the variable light chain set forth in SEQ ID NO: 282 or a sequence of amino acids that exhibits at least 85% sequence identity to SEQ ID NO: 282;

hh) the variable heavy chain set forth in SEQ ID NO: 213 or a sequence of amino acids that exhibits at least 85% sequence identity to SEQ ID NO: 213, and the variable light chain set forth in SEQ ID NO: 289 or a sequence of amino acids that exhibits at least 85% sequence identity to SEQ ID NO: 289.

In other examples, an unmodified anti-EGFR antibody or antigen-binding fragment thereof, that has a variable heavy chain set forth in SEQ ID NO: 7 and a variable light chain set forth in SEQ ID NO: 9, is humanized and modified to generate a modified anti-EGFR antibody, or antigen-binding fragment thereof, that contains:

a) the variable heavy chain set forth in SEQ ID NO: 131 or a sequence of amino acids that exhibits at least 85% sequence identity to SEQ ID NO: 131, and the variable light chain set forth in SEQ ID NO: 155 or a sequence of amino acids that exhibits at least 85% sequence identity to SEQ ID NO: 155;

b) the variable heavy chain set forth in SEQ ID NO: 131 or a sequence of amino acids that exhibits at least 85% sequence identity to SEQ ID NO: 131, and the variable light chain set forth in SEQ ID NO: 162, or a sequence of amino acids that exhibits at least 85% sequence identity to SEQ ID NO: 162;

c) the variable heavy chain set forth in SEQ ID NO: 137 or a sequence of amino acids that exhibits at least 85% sequence identity to SEQ ID NO: 137, and the variable light chain set forth in SEQ ID NO: 155 or a sequence of amino acids that exhibits at least 85% sequence identity to SEQ ID NO: 155;

d) the variable heavy chain set forth in SEQ ID NO: 131 or a sequence of amino acids that exhibits at least 85% sequence identity to SEQ ID NO: 131, and the variable light chain set forth in SEQ ID NO: 169 or a sequence of amino acids that exhibits at least 85% sequence identity to SEQ ID NO: 169;

e) the variable heavy chain set forth in SEQ ID NO: 131 or a sequence of amino acids that exhibits at least 85% sequence identity to SEQ ID NO: 131, and the variable light chain set forth in SEQ ID NO: 176 or a sequence of amino acids that exhibits at least 85% sequence identity to SEQ ID NO: 176;

f) the variable heavy chain set forth in SEQ ID NO: 131 or a sequence of amino acids that exhibits at least 85% sequence identity to SEQ ID NO: 131 and the variable light chain set forth in SEQ ID NO: 183 or a sequence of amino acids that exhibits at least 85% sequence identity to SEQ ID NO: 183;

g) the variable heavy chain set forth in SEQ ID NO: 137 or a sequence of amino acids that exhibits at least 85% sequence identity to SEQ ID NO: 137, and the variable light chain set forth in SEQ ID NO: 183 or a sequence of amino acids that exhibits at least 85% sequence identity to SEQ ID NO: 183;

h) the variable heavy chain set forth in SEQ ID NO: 131 or a sequence of amino acids that exhibits at least 85% sequence identity to SEQ ID NO: 131, and the variable light chain set forth in SEQ ID NO: 190 or a sequence of amino acids that exhibits at least 85% sequence identity to SEQ ID NO: 190;

i) the variable heavy chain set forth in SEQ ID NO: 143 or a sequence of amino acids that exhibits at least 85% sequence identity to SEQ ID NO: 143, and the variable light chain set forth in SEQ ID NO: 183 or a sequence of amino acids that exhibits at least 85% sequence identity to SEQ ID NO: 183;

j) the variable heavy chain set forth in SEQ ID NO: 149 or a sequence of amino acids that exhibits at least 85% sequence identity to SEQ ID NO: 149, and the variable light chain set forth in SEQ ID NO: 197 or a sequence of amino acids that exhibits at least 85% sequence identity to SEQ ID NO: 197;

k) the variable heavy chain set forth in SEQ ID NO: 143 or a sequence of amino acids that exhibits at least 85% sequence identity to SEQ ID NO: 143, and the variable light chain set forth in SEQ ID NO: 197 or a sequence of amino acids that exhibits at least 85% sequence identity to SEQ ID NO: 197;

l) the variable heavy chain set forth in SEQ ID NO: 149 or a sequence of amino acids that exhibits at least 85% sequence identity to SEQ ID NO: 149, and the variable light chain set forth in SEQ ID NO: 204 or a sequence of amino acids that exhibits at least 85% sequence identity to SEQ ID NO: 204;

m) the variable heavy chain set forth in SEQ ID NO: 143 or a sequence of amino acids that exhibits at least 85% sequence identity to SEQ ID NO: 143, and the variable light chain set forth in SEQ ID NO: 204 or a sequence of amino acids that exhibits at least 85% sequence identity to SEQ ID NO: 204;

n) the variable heavy chain set forth in SEQ ID NO: 211 or a sequence of amino acids that exhibits at least 85% sequence identity to SEQ ID NO: 211, and the variable light chain set forth in SEQ ID NO: 253 or a sequence of amino acids that exhibits at least 85% sequence identity to SEQ ID NO: 253;

o) the variable heavy chain set forth in SEQ ID NO: 217 or a sequence of amino acids that exhibits at least 85% sequence identity to SEQ ID NO: 217, and the variable light chain set forth in SEQ ID NO: 253 or a sequence of amino acids that exhibits at least 85% sequence identity to SEQ ID NO: 253;

p) the variable heavy chain set forth in SEQ ID NO: 223 or a sequence of amino acids that exhibits at least 85% sequence identity to SEQ ID NO: 223, and the variable light chain set forth in SEQ ID NO: 260 or a sequence of amino acids that exhibits at least 85% sequence identity to SEQ ID NO: 260;

q) the variable heavy chain set forth in SEQ ID NO: 229 or a sequence of amino acids that exhibits at least 85% sequence identity to SEQ ID NO: 229, and the variable light chain set forth in SEQ ID NO: 260 or a sequence of amino acids that exhibits at least 85% sequence identity to SEQ ID NO: 260;

r) the variable heavy chain set forth in SEQ ID NO: 235 or a sequence of amino acids that exhibits at least 85% sequence identity to SEQ ID NO: 235, and the variable light chain set forth in SEQ ID NO: 267 or a sequence of amino acids that exhibits at least 85% sequence identity to SEQ ID NO: 267;

s) the variable heavy chain set forth in SEQ ID NO: 241 or a sequence of amino acids that exhibits at least 85% sequence identity to SEQ ID NO: 241, and the variable light chain set forth in SEQ ID NO: 274 or a sequence of amino acids that exhibits at least 85% sequence identity to SEQ ID NO: 274;

t) the variable heavy chain set forth in SEQ ID NO: 223 or a sequence of amino acids that exhibits at least 85% sequence identity to SEQ ID NO: 223, and the variable light chain set forth in SEQ ID NO: 274 or a sequence of amino acids that exhibits at least 85% sequence identity to SEQ ID NO: 274;

u) the variable heavy chain set forth in SEQ ID NO: 229 or a sequence of amino acids that exhibits at least 85% sequence identity to SEQ ID NO: 229, and the variable light chain set forth in SEQ ID NO: 274 or a sequence of amino acids that exhibits at least 85% sequence identity to SEQ ID NO: 274;

v) the variable heavy chain set forth in SEQ ID NO: 235 or a sequence of amino acids that exhibits at least 85% sequence identity to SEQ ID NO: 235, and the variable light chain set forth in SEQ ID NO: 281 or a sequence of amino acids that exhibits at least 85% sequence identity to SEQ ID NO: 281;

w) the variable heavy chain set forth in SEQ ID NO: 247 or a sequence of amino acids that exhibits at least 85% sequence identity to SEQ ID NO: 247, and the variable light chain set forth in SEQ ID NO: 281 or a sequence of amino acids that exhibits at least 85% sequence identity to SEQ ID NO: 281;

x) the variable heavy chain set forth in SEQ ID NO: 223 or a sequence of amino acids that exhibits at least 85% sequence identity to SEQ ID NO: 223, and the variable light chain set forth in SEQ ID NO: 281 or a sequence of amino acids that exhibits at least 85% sequence identity to SEQ ID NO: 281;

y) the variable heavy chain set forth in SEQ ID NO: 229 or a sequence of amino acids that exhibits at least 85% sequence identity to SEQ ID NO: 229, and the variable light chain set forth in SEQ ID NO: 281 or a sequence of amino acids that exhibits at least 85% sequence identity to SEQ ID NO: 281;

z) the variable heavy chain set forth in SEQ ID NO: 235 or a sequence of amino acids that exhibits at least 85% sequence identity to SEQ ID NO: 235, and the variable light chain set forth in SEQ ID NO: 288 or a sequence of amino acids that exhibits at least 85% sequence identity to SEQ ID NO: 288;

aa) the variable heavy chain set forth in SEQ ID NO: 247 or a sequence of amino acids that exhibits at least 85% sequence identity to SEQ ID NO: 247, and the variable light chain set forth in SEQ ID NO: 288 or a sequence of amino acids that exhibits at least 85% sequence identity to SEQ ID NO: 288;

bb) the variable heavy chain set forth in SEQ ID NO: 223 or a sequence of amino acids that exhibits at least 85% sequence identity to SEQ ID NO: 223, and the variable light chain set forth in SEQ ID NO: 288 or a sequence of amino acids that exhibits at least 85% sequence identity to SEQ ID NO: 288;

cc) the variable heavy chain set forth in SEQ ID NO: 229 or a sequence of amino acids that exhibits at least 85% sequence identity to SEQ ID NO: 229, and the variable light chain set forth in SEQ ID NO: 288 or a sequence of amino acids that exhibits at least 85% sequence identity to SEQ ID NO: 288;

dd) the variable heavy chain set forth in SEQ ID NO: 235 or a sequence of amino acids that exhibits at least 85% sequence identity to SEQ ID NO: 235, and the variable light chain set forth in SEQ ID NO: 295 or a sequence of amino acids that exhibits at least 85% sequence identity to SEQ ID NO: 295;

ee) the variable heavy chain set forth in SEQ ID NO: 247 or a sequence of amino acids that exhibits at least 85% sequence identity to SEQ ID NO: 247, and the variable light chain set forth in SEQ ID NO: 302 or a sequence of amino acids that exhibits at least 85% sequence identity to SEQ ID NO: 302;

ff) the variable heavy chain set forth in SEQ ID NO: 211 or a sequence of amino acids that exhibits at least 85% sequence identity to SEQ ID NO: 211, and the variable light chain set forth in SEQ ID NO: 302 or a sequence of amino acids that exhibits at least 85% sequence identity to SEQ ID NO: 302;

gg) the variable heavy chain set forth in SEQ ID NO: 211 or a sequence of amino acids that exhibits at least 85% sequence identity to SEQ ID NO: 211, and the variable light chain set forth in SEQ ID NO: 281 or a sequence of amino acids that exhibits at least 85% sequence identity to SEQ ID NO: 281; and

hh) the variable heavy chain set forth in SEQ ID NO: 211 or a sequence of amino acids that exhibits at least 85% sequence identity to SEQ ID NO: 211, and the variable light chain set forth in SEQ ID NO: 288 or a sequence of amino acids that exhibits at least 85% sequence identity to SEQ ID NO: 288.

In further examples, an unmodified anti-EGFR antibody or antigen-binding fragment thereof, that has a variable heavy chain set forth in SEQ ID NO: 7 and a variable light chain set forth in SEQ ID NO: 11, is humanized and modified to generate a modified anti-EGFR antibody, or antigen-binding fragment thereof, that contains:

a) the variable heavy chain set forth in SEQ ID NO: 131 or a sequence of amino acids that exhibits at least 85% sequence identity to SEQ ID NO: 131, and the variable light chain set forth in SEQ ID NO: 158 or a sequence of amino acids that exhibits at least 85% sequence identity to SEQ ID NO: 158;

b) the variable heavy chain set forth in SEQ ID NO: 131 or a sequence of amino acids that exhibits at least 85% sequence identity to SEQ ID NO: 131, and the variable light chain set forth in SEQ ID NO: 165, or a sequence of amino acids that exhibits at least 85% sequence identity to SEQ ID NO: 165;

c) the variable heavy chain set forth in SEQ ID NO: 137 or a sequence of amino acids that exhibits at least 85% sequence identity to SEQ ID NO: 137, and the variable light chain set forth in SEQ ID NO: 158 or a sequence of amino acids that exhibits at least 85% sequence identity to SEQ ID NO: 158;

d) the variable heavy chain set forth in SEQ ID NO: 131 or a sequence of amino acids that exhibits at least 85% sequence identity to SEQ ID NO: 131, and the variable light chain set forth in SEQ ID NO: 172 or a sequence of amino acids that exhibits at least 85% sequence identity to SEQ ID NO: 172;

e) the variable heavy chain set forth in SEQ ID NO: 131 or a sequence of amino acids that exhibits at least 85% sequence identity to SEQ ID NO: 131, and the variable light chain set forth in SEQ ID NO: 179 or a sequence of amino acids that exhibits at least 85% sequence identity to SEQ ID NO: 179;

f) the variable heavy chain set forth in SEQ ID NO: 131 or a sequence of amino acids that exhibits at least 85% sequence identity to SEQ ID NO: 131 and the variable light chain set forth in SEQ ID NO: 186 or a sequence of amino acids that exhibits at least 85% sequence identity to SEQ ID NO: 186;

g) the variable heavy chain set forth in SEQ ID NO: 137 or a sequence of amino acids that exhibits at least 85% sequence identity to SEQ ID NO: 137, and the variable light chain set forth in SEQ ID NO: 186 or a sequence of amino acids that exhibits at least 85% sequence identity to SEQ ID NO: 186;

h) the variable heavy chain set forth in SEQ ID NO: 131 or a sequence of amino acids that exhibits at least 85% sequence identity to SEQ ID NO: 131, and the variable light chain set forth in SEQ ID NO: 193 or a sequence of amino acids that exhibits at least 85% sequence identity to SEQ ID NO: 193;

i) the variable heavy chain set forth in SEQ ID NO: 143 or a sequence of amino acids that exhibits at least 85% sequence identity to SEQ ID NO: 143, and the variable light chain set forth in SEQ ID NO: 186 or a sequence of amino acids that exhibits at least 85% sequence identity to SEQ ID NO: 186;

j) the variable heavy chain set forth in SEQ ID NO: 149 or a sequence of amino acids that exhibits at least 85% sequence identity to SEQ ID NO: 149, and the variable light chain set forth in SEQ ID NO: 200 or a sequence of amino acids that exhibits at least 85% sequence identity to SEQ ID NO: 200;

k) the variable heavy chain set forth in SEQ ID NO: 143 or a sequence of amino acids that exhibits at least 85% sequence identity to SEQ ID NO: 143, and the variable light chain set forth in SEQ ID NO: 200 or a sequence of amino acids that exhibits at least 85% sequence identity to SEQ ID NO: 200;

l) the variable heavy chain set forth in SEQ ID NO: 149 or a sequence of amino acids that exhibits at least 85% sequence identity to SEQ ID NO: 149, and the variable light chain set forth in SEQ ID NO: 207 or a sequence of amino acids that exhibits at least 85% sequence identity to SEQ ID NO: 207;

m) the variable heavy chain set forth in SEQ ID NO: 143 or a sequence of amino acids that exhibits at least 85% sequence identity to SEQ ID NO: 143, and the variable light chain set forth in SEQ ID NO: 207 or a sequence of amino acids that exhibits at least 85% sequence identity to SEQ ID NO: 207;

n) the variable heavy chain set forth in SEQ ID NO: 211 or a sequence of amino acids that exhibits at least 85% sequence identity to SEQ ID NO: 211, and the variable light chain set forth in SEQ ID NO: 256 or a sequence of amino acids that exhibits at least 85% sequence identity to SEQ ID NO: 256;

o) the variable heavy chain set forth in SEQ ID NO: 217 or a sequence of amino acids that exhibits at least 85% sequence identity to SEQ ID NO: 217, and the variable light chain set forth in SEQ ID NO: 256 or a sequence of amino acids that exhibits at least 85% sequence identity to SEQ ID NO: 256;

p) the variable heavy chain set forth in SEQ ID NO: 223 or a sequence of amino acids that exhibits at least 85% sequence identity to SEQ ID NO: 223, and the variable light chain set forth in SEQ ID NO: 263 or a sequence of amino acids that exhibits at least 85% sequence identity to SEQ ID NO: 263;

q) the variable heavy chain set forth in SEQ ID NO: 229 or a sequence of amino acids that exhibits at least 85% sequence identity to SEQ ID NO: 229, and the variable light chain set forth in SEQ ID NO: 263 or a sequence of amino acids that exhibits at least 85% sequence identity to SEQ ID NO: 263;

r) the variable heavy chain set forth in SEQ ID NO: 235 or a sequence of amino acids that exhibits at least 85% sequence identity to SEQ ID NO: 235, and the variable light chain set forth in SEQ ID NO: 270 or a sequence of amino acids that exhibits at least 85% sequence identity to SEQ ID NO: 270;

s) the variable heavy chain set forth in SEQ ID NO: 241 or a sequence of amino acids that exhibits at least 85% sequence identity to SEQ ID NO: 241, and the variable light chain set forth in SEQ ID NO: 277 or a sequence of amino acids that exhibits at least 85% sequence identity to SEQ ID NO: 277;

t) the variable heavy chain set forth in SEQ ID NO: 223 or a sequence of amino acids that exhibits at least 85% sequence identity to SEQ ID NO: 223, and the variable light chain set forth in SEQ ID NO: 277 or a sequence of amino acids that exhibits at least 85% sequence identity to SEQ ID NO: 277;

u) the variable heavy chain set forth in SEQ ID NO: 229 or a sequence of amino acids that exhibits at least 85% sequence identity to SEQ ID NO: 229, and the variable light chain set forth in SEQ ID NO: 277 or a sequence of amino acids that exhibits at least 85% sequence identity to SEQ ID NO: 277;

v) the variable heavy chain set forth in SEQ ID NO: 235 or a sequence of amino acids that exhibits at least 85% sequence identity to SEQ ID NO: 235, and the variable light chain set forth in SEQ ID NO: 284 or a sequence of amino acids that exhibits at least 85% sequence identity to SEQ ID NO: 284;

w) the variable heavy chain set forth in SEQ ID NO: 247 or a sequence of amino acids that exhibits at least 85% sequence identity to SEQ ID NO: 247, and the variable light chain set forth in SEQ ID NO: 284 or a sequence of amino acids that exhibits at least 85% sequence identity to SEQ ID NO: 284;

x) the variable heavy chain set forth in SEQ ID NO: 223 or a sequence of amino acids that exhibits at least 85% sequence identity to SEQ ID NO: 223, and the variable light chain set forth in SEQ ID NO: 284 or a sequence of amino acids that exhibits at least 85% sequence identity to SEQ ID NO: 284;

y) the variable heavy chain set forth in SEQ ID NO: 229 or a sequence of amino acids that exhibits at least 85% sequence identity to SEQ ID NO: 229, and the variable light chain set forth in SEQ ID NO: 284 or a sequence of amino acids that exhibits at least 85% sequence identity to SEQ ID NO: 284;

z) the variable heavy chain set forth in SEQ ID NO: 235 or a sequence of amino acids that exhibits at least 85% sequence identity to SEQ ID NO: 235, and the variable light chain set forth in SEQ ID NO: 291 or a sequence of amino acids that exhibits at least 85% sequence identity to SEQ ID NO: 291;

aa) the variable heavy chain set forth in SEQ ID NO: 247 or a sequence of amino acids that exhibits at least 85% sequence identity to SEQ ID NO: 247, and the variable light chain set forth in SEQ ID NO: 291 or a sequence of amino acids that exhibits at least 85% sequence identity to SEQ ID NO: 291;

bb) the variable heavy chain set forth in SEQ ID NO: 223 or a sequence of amino acids that exhibits at least 85% sequence identity to SEQ ID NO: 223, and the variable light chain set forth in SEQ ID NO: 291 or a sequence of amino acids that exhibits at least 85% sequence identity to SEQ ID NO: 291;

cc) the variable heavy chain set forth in SEQ ID NO: 229 or a sequence of amino acids that exhibits at least 85% sequence identity to SEQ ID NO: 229, and the variable light chain set forth in SEQ ID NO: 291 or a sequence of amino acids that exhibits at least 85% sequence identity to SEQ ID NO: 291;

dd) the variable heavy chain set forth in SEQ ID NO: 235 or a sequence of amino acids that exhibits at least 85% sequence identity to SEQ ID NO: 235, and the variable light chain set forth in SEQ ID NO: 298 or a sequence of amino acids that exhibits at least 85% sequence identity to SEQ ID NO: 298;

ee) the variable heavy chain set forth in SEQ ID NO: 247 or a sequence of amino acids that exhibits at least 85% sequence identity to SEQ ID NO: 247, and the variable light chain set forth in SEQ ID NO: 305 or a sequence of amino acids that exhibits at least 85% sequence identity to SEQ ID NO: 305;

ff) the variable heavy chain set forth in SEQ ID NO: 211 or a sequence of amino acids that exhibits at least 85% sequence identity to SEQ ID NO: 211, and the variable light chain set forth in SEQ ID NO: 305 or a sequence of amino acids that exhibits at least 85% sequence identity to SEQ ID NO: 305;

gg) the variable heavy chain set forth in SEQ ID NO: 211 or a sequence of amino acids that exhibits at least 85% sequence identity to SEQ ID NO: 211, and the variable light chain set forth in SEQ ID NO: 284 or a sequence of amino acids that exhibits at least 85% sequence identity to SEQ ID NO: 284;

hh) the variable heavy chain set forth in SEQ ID NO: 211 or a sequence of amino acids that exhibits at least 85% sequence identity to SEQ ID NO: 211, and the variable light chain set forth in SEQ ID NO: 291 or a sequence of amino acids that exhibits at least 85% sequence identity to SEQ ID NO: 291.

Also included among any of modified anti-EGFR antibodies, or antigen-binding fragments thereof, are any humanized, modified anti-EGFR antibodies, or antigen-binding fragments thereof, that exhibit at least 86%, 87%, 88%, 89%, 90%, 91%, 92%, 93%, 94%, 95%, 96%, 97%, 98%, 99% or more sequence identity to any of the humanized, modified anti-EGFR antibodies described herein above. Sequence identity can be determined using global alignment with or without gaps.

In some examples, the humanized, modified anti-EGFR antibody, or antigen-binding fragment thereof, is a full-length antibody that contains:

a) the heavy chain set forth in SEQ ID NO: 129 or a sequence of amino acids that exhibits at least 85% sequence identity to SEQ ID NO: 129, and the light chain set forth in SEQ ID NO: 153 or a sequence of amino acids that exhibits at least 85% sequence identity to SEQ ID NO: 153;

b) the heavy chain set forth in SEQ ID NO: 129 or a sequence of amino acids that exhibits at least 85% sequence identity to SEQ ID NO: 129, and the light chain set forth in SEQ ID NO: 160 or a sequence of amino acids that exhibits at least 85% sequence identity to SEQ ID NO: 160;

c) the heavy chain set forth in SEQ ID NO: 135 or a sequence of amino acids that exhibits at least 85% sequence identity to SEQ ID NO: 135, and the light chain set forth in SEQ ID NO: 153 or a sequence of amino acids that exhibits at least 85% sequence identity to SEQ ID NO: 153;

d) the heavy chain set forth in SEQ ID NO: 129 or a sequence of amino acids that exhibits at least 85% sequence identity to SEQ ID NO: 129, and the light chain set forth in SEQ ID NO: 167 or a sequence of amino acids that exhibits at least 85% sequence identity to SEQ ID NO: 167;

e) the heavy chain set forth in SEQ ID NO: 129 or a sequence of amino acids that exhibits at least 85% sequence identity to SEQ ID NO: 129, and the light chain set forth in SEQ ID NO: 174 or a sequence of amino acids that exhibits at least 85% sequence identity to SEQ ID NO: 174;

f) the heavy chain set forth in SEQ ID NO: 129 or a sequence of amino acids that exhibits at least 85% sequence identity to SEQ ID NO: 129, and the light chain set forth in SEQ ID NO: 181 or a sequence of amino acids that exhibits at least 85% sequence identity to SEQ ID NO: 181;

g) the heavy chain set forth in SEQ ID NO: 135 or a sequence of amino acids that exhibits at least 85% sequence identity to SEQ ID NO: 135, and the light chain set forth in SEQ ID NO: 181 or a sequence of amino acids that exhibits at least 85% sequence identity to SEQ ID NO: 181;

h) the heavy chain set forth in SEQ ID NO: 129 or a sequence of amino acids that exhibits at least 85% sequence identity to SEQ ID NO: 129, and the light chain set forth in SEQ ID NO: 188 or a sequence of amino acids that exhibits at least 85% sequence identity to SEQ ID NO: 188;

i) the heavy chain set forth in SEQ ID NO: 141 or a sequence of amino acids that exhibits at least 85% sequence identity to SEQ ID NO: 141, and the light chain set forth in SEQ ID NO: 181 or a sequence of amino acids that exhibits at least 85% sequence identity to SEQ ID NO: 181;

j) the heavy chain set forth in SEQ ID NO: 147 or a sequence of amino acids that exhibits at least 85% sequence identity to SEQ ID NO: 147, and the light chain set forth in SEQ ID NO: 195 or a sequence of amino acids that exhibits at least 85% sequence identity to SEQ ID NO: 195;

k) the heavy chain set forth in SEQ ID NO: 141 or a sequence of amino acids that exhibits at least 85% sequence identity to SEQ ID NO: 141, and the light chain set forth in SEQ ID NO: 195 or a sequence of amino acids that exhibits at least 85% sequence identity to SEQ ID NO: 195;

l) the heavy chain set forth in SEQ ID NO: 147 or a sequence of amino acids that exhibits at least 85% sequence identity to SEQ ID NO: 147, and the light chain set forth in SEQ ID NO: 202 or a sequence of amino acids that exhibits at least 85% sequence identity to SEQ ID NO: 202;

m) the heavy chain set forth in SEQ ID NO: 141 or a sequence of amino acids that exhibits at least 85% sequence identity to SEQ ID NO: 141, and the light chain set forth in SEQ ID NO: 202 or a sequence of amino acids that exhibits at least 85% sequence identity to SEQ ID NO: 202;

n) the heavy chain set forth in SEQ ID NO: 209 or a sequence of amino acids that exhibits at least 85% sequence identity to SEQ ID NO: 209, and the light chain set forth in SEQ ID NO: 251 or a sequence of amino acids that exhibits at least 85% sequence identity to SEQ ID NO: 251;

o) the heavy chain set forth in SEQ ID NO: 215 or a sequence of amino acids that exhibits at least 85% sequence identity to SEQ ID NO: 215, and the light chain set forth in SEQ ID NO: 251 or a sequence of amino acids that exhibits at least 85% sequence identity to SEQ ID NO: 251;

p) the heavy chain set forth in SEQ ID NO: 221 or a sequence of amino acids that exhibits at least 85% sequence identity to SEQ ID NO: 221, and the light chain set forth in SEQ ID NO: 258 or a sequence of amino acids that exhibits at least 85% sequence identity to SEQ ID NO: 258;

q) the heavy chain set forth in SEQ ID NO: 227 or a sequence of amino acids that exhibits at least 85% sequence identity to SEQ ID NO: 227, and the light chain set forth in SEQ ID NO: 258 or a sequence of amino acids that exhibits at least 85% sequence identity to SEQ ID NO: 258;

r) the heavy chain set forth in SEQ ID NO: 233 or a sequence of amino acids that exhibits at least 85% sequence identity to SEQ ID NO: 233, and the light chain set forth in SEQ ID NO: 265 or a sequence of amino acids that exhibits at least 85% sequence identity to SEQ ID NO: 265;

s) the heavy chain set forth in SEQ ID NO: 239 or a sequence of amino acids that exhibits at least 85% sequence identity to SEQ ID NO: 239, and the light chain set forth in SEQ ID NO: 272 or a sequence of amino acids that exhibits at least 85% sequence identity to SEQ ID NO: 272;

t) the heavy chain set forth in SEQ ID NO: 221 or a sequence of amino acids that exhibits at least 85% sequence identity to SEQ ID NO: 221, and the light chain set forth in SEQ ID NO: 272 or a sequence of amino acids that exhibits at least 85% sequence identity to SEQ ID NO: 272;

u) the heavy chain set forth in SEQ ID NO: 227 or a sequence of amino acids that exhibits at least 85% sequence identity to SEQ ID NO: 227, and the light chain set forth in SEQ ID NO: 272 or a sequence of amino acids that exhibits at least 85% sequence identity to SEQ ID NO: 272;

v) the heavy chain set forth in SEQ ID NO: 233 or a sequence of amino acids that exhibits at least 85% sequence identity to SEQ ID NO: 233, and the light chain set forth in SEQ ID NO: 279 or a sequence of amino acids that exhibits at least 85% sequence identity to SEQ ID NO: 279;

w) the heavy chain set forth in SEQ ID NO: 245 or a sequence of amino acids that exhibits at least 85% sequence identity to SEQ ID NO: 245, and the light chain set forth in SEQ ID NO: 279 or a sequence of amino acids that exhibits at least 85% sequence identity to SEQ ID NO: 279;

x) the heavy chain set forth in SEQ ID NO: 221 or a sequence of amino acids that exhibits at least 85% sequence identity to SEQ ID NO: 221, and the light chain set forth in SEQ ID NO: 279 or a sequence of amino acids that exhibits at least 85% sequence identity to SEQ ID NO: 279;

y) the heavy chain set forth in SEQ ID NO: 227 or a sequence of amino acids that exhibits at least 85% sequence identity to SEQ ID NO: 227, and the light chain set forth in SEQ ID NO: 279 or a sequence of amino acids that exhibits at least 85% sequence identity to SEQ ID NO: 279;

z) the heavy chain set forth in SEQ ID NO: 233 or a sequence of amino acids that exhibits at least 85% sequence identity to SEQ ID NO: 233, and the light chain set forth in SEQ ID NO: 286 or a sequence of amino acids that exhibits at least 85% sequence identity to SEQ ID NO: 286;

aa) the heavy chain set forth in SEQ ID NO: 245 or a sequence of amino acids that exhibits at least 85% sequence identity to SEQ ID NO: 245, and the light chain set forth in SEQ ID NO: 286 or a sequence of amino acids that exhibits at least 85% sequence identity to SEQ ID NO: 286;

bb) the heavy chain set forth in SEQ ID NO: 221 or a sequence of amino acids that exhibits at least 85% sequence identity to SEQ ID NO: 221, and the light chain set forth in SEQ ID NO: 286 or a sequence of amino acids that exhibits at least 85% sequence identity to SEQ ID NO: 286;

cc) the heavy chain set forth in SEQ ID NO: 227 or a sequence of amino acids that exhibits at least 85% sequence identity to SEQ ID NO: 227, and the light chain set forth in SEQ ID NO: 286 or a sequence of amino acids that exhibits at least 85% sequence identity to SEQ ID NO: 286;

dd) the heavy chain set forth in SEQ ID NO: 233 or a sequence of amino acids that exhibits at least 85% sequence identity to SEQ ID NO: 233, and the light chain set forth in SEQ ID NO: 293 or a sequence of amino acids that exhibits at least 85% sequence identity to SEQ ID NO: 293;

ee) the heavy chain set forth in SEQ ID NO: 245 or a sequence of amino acids that exhibits at least 85% sequence identity to SEQ ID NO: 245, and the light chain set forth in SEQ ID NO: 300 or a sequence of amino acids that exhibits at least 85% sequence identity to SEQ ID NO: 300;

ff) the heavy chain set forth in SEQ ID NO: 209 or a sequence of amino acids that exhibits at least 85% sequence identity to SEQ ID NO: 209, and the light chain set forth in SEQ ID NO: 300 or a sequence of amino acids that exhibits at least 85% sequence identity to SEQ ID NO: 300;

gg) the heavy chain set forth in SEQ ID NO: 209 or a sequence of amino acids that exhibits at least 85% sequence identity to SEQ ID NO: 209, and the light chain set forth in SEQ ID NO: 279 or a sequence of amino acids that exhibits at least 85% sequence identity to SEQ ID NO: 279; and

hh) the heavy chain set forth in SEQ ID NO: 209 or a sequence of amino acids that exhibits at least 85% sequence identity to SEQ ID NO: 209, and the light chain set forth in SEQ ID NO: 286 or a sequence of amino acids that exhibits at least 85% sequence identity to SEQ ID NO: 286.

In any of the examples of modified anti-EGFR antibodies, or antigen-binding fragments thereof, provided herein, the antibody or antigen binding fragment can exhibit a ratio of binding activity for EGFR of greater than 1.0 in the presence of one or both of a pH that is pH 6.0 to 6.5, inclusive, an/or a lactate concentration of 15 mM to 20 mM, inclusive, compared to in the presence of one or both of or about pH 7.4 and/or a lactate concentration of or about 1 mM, when measured under the same conditions except for the difference in pH and lactate concentration. In some examples, the modified anti-EGFR antibody exhibits a ratio of binding activity for EGFR of greater than 1.0 in the presence of a pH that is pH 6.0 to 6.5, inclusive, compared to in the presence of or about pH 7.4, when measured under the same conditions except for the difference in pH. In such examples, the ratio of binding activity can be at least 1.1, 1.2, 1.3, 1.4, 1.5, 1.6, 1.7, 1.8, 1.9, 2.0, 2.5, 3.0, 4.0, 4.5, 5.0, 6.0. 7.0, 8.0, 9.0, 10.0, 15.0, 20.0, 25.0, 30.0, 35.0, 40.0, 50.0 or greater. In particular examples, the ratio of binding activity is at least 3.0, 4.0, 5.0, 6.0. 7.0, 8.0, 9.0, 10.0, 15.0, 20.0, 25.0, 30.0, 35.0, 40.0, 50.0 or greater.

In any of the examples herein, the binding affinity of the modified anti-EGFR antibodies or antigen-binding fragments provided herein is measured in terms of the dissociation constant (K_{d}) for binding EGFR or a soluble fragment thereof. In such examples, the modified anti-EGFR antibodies or antigen-binding fragments provided herein can have a binding affinity (K_{d}) for EGFR that is less than 1×10⁻⁸ M, 5×10⁻⁹ M, 1×10⁻⁹M, 5×10⁻¹⁰ M, 1×10⁻¹⁰ M, 5×10⁻¹¹ M, 1×10⁻¹¹ M or less under conditions that include one or both of acidic pH 6.0 to 6.5, inclusive, and 15 mM to 20 mM lactate, inclusive; and/or a K_{d} for EGFR that is greater than 1×10⁻⁸ M, 1×10⁻⁷ M, 1×10⁻⁶M, or greater under conditions that include one or both of or about pH 7.4 and 1 mM lactate, inclusive.

In any of the examples herein, the binding affinity of the modified anti-EGFR antibodies or antigen-binding fragments provided herein is measured in terms of half-maximal effective concentration (EC₅₀). In such examples, the modified anti-EGFR antibody, or antigen-binding fragment thereof, exhibits binding activity with an EC₅₀ for binding EGFR, or a soluble fragment thereof, that is less than 10 mM, 5 mM, 4 mM, 3 mM, 2 mM, 1 mM or less under conditions that include one or both of acidic pH (pH 6.0 to 6.5, inclusive) and/or 15 mM to 20 mM lactate, inclusive; and/or an EC₅₀ for binding EGFR, or a soluble fragment thereof, that is greater than 5 mM, 10 mM, 15 mM, 20 mM, 25 mM, 30 mM, 40 mM, 50 mM, 60 mM or greater.

In any of the examples herein, the binding activity of the modified anti-EGFR antibody, or antigen-binding fragment thereof, can be measured in the presence of a protein concentration that is at least 12 mg/mL, 15 mg/mL, 20 mg/mL, 25 mg/mL, 30 mg/mL, 35 mg/mL, 40 mg/mL, 45 mg/mL or 50 mg/mL, which for example, can be provided in serum, such as human serum, or as a serum albumin, such as human serum albumin.

In any of the examples herein, the protein is provided in serum and binding assays to test the binding activity of the modified anti-EGFR antibodies and fragments provided herein are performed in the presence of 20% (vol/vol) to 90% (vol/vol) serum, such as 20% (vol/vol) to 50% (vol/vol) or 20% (vol/vol) to 40% (vol/vol) serum. In particular examples, binding assays are performed in the presence of 25% (vol/vol) serum or about 25% (vol/vol) serum, such as human serum.

The variable heavy chain of the modified anti-EGFR antibodies or antigen binding fragments thereof can contain one or more amino acid replacements compared to the amino acid sequence of an unmodified anti-EGFR antibody, including 1 to 50 amino acid replacements, such as 1 to 40, 1 to 30, 1 to 20, 1 to 10 or 1 to 5 amino acid replacements compared to the unmodified variable heavy chain, such as the unmodified variable heavy chain set forth in SEQ ID NO: 2 or 7.

Any of the anti-EGFR antibodies or EGFR-binding fragments provided herein can be isolated or purified after production.

Provided herein are conjugates containing any of the anti-EGFR antibodies, or antigen-binding fragments thereof, provided herein, linked directly or indirectly to a targeted agent. Such conjugates contain the anti-EGFR antibody or antigen-binding fragment thereof that binds to EGFR (Ab), one or more targeted agent, and an optional a linker (L) for linking the Ab to the targeted agent. In some examples, there are 1 to 8 targeted agents conjugated to the antibody by 0 to 8 linkers.

The targeted agent of the conjugate can be a protein, peptide, nucleic acid or small molecule. In particular examples, the targeted agent is a therapeutic moiety, such as a cytotoxic moiety, a radioisotope, a chemotherapeutic agent, a lytic peptide or a cytokine. Exemplary therapeutic moieties which can be conjugated to any of the modified anti-EGFR antibodies, or fragments thereof, provided herein include, taxol; cytochalasin B; gramicidin D; ethidium bromide; emetine; mitomycin; etoposide; teniposide; vincristine; vinblastine; colchicine; doxorubicin; daunorubicin; dihydroxy anthracin dione; maytansine or an analog or derivative thereof; an auristatin or a functional peptide analog or derivative thereof; dolastatin 10 or 15 or an analog thereof; irinotecan or an analog thereof; mitoxantrone; mithramycin; actinomycin D; 1-dehydrotestosterone; a glucocorticoid; procaine; tetracaine; lidocaine; propranolol; puromycin; calicheamicin or an analog or derivative thereof; an antimetabolite; an alkylating agent; a platinum derivative; duocarmycin A, duocarmycin SA, rachelmycin (CC-1065), or an analog or derivative thereof; an antibiotic; a pyrrolo[2,1-c][1, 4]-benzodiazepine (PBD); a toxin; ribonuclease (RNase); DNase I, Staphylococcal enterotoxin A; and pokeweed antiviral protein.

In particular examples, the therapeutic moiety is a maytansine derivative that is a maytansinoid, such as ansamitocin or mertansine (DM1); an auristatin or a functional peptide analog or derivative thereof, such as monomethyl auristatin E (MMAE) or F (MMAF); an antimetabolite, such as methotrexate, 6-mercaptopurine, 6-thioguanine, cytarabine, fludarabine, 5-fluorouracil, decarbazine, hydroxyurea, asparaginase, gemcitabine, or cladribine; alkylating agent, such as mechlorethamine, thiotepa, chlorambucil, melphalan, carmustine (BCNU), lomustine (CCNU), cyclophosphamide, busulfan, dibromomannitol, streptozotocin, dacarbazine (DTIC), procarbazine and mitomycin C; a platinum derivative, such as cisplatin or carboplatin; an antibiotic, such as dactinomycin, bleomycin, daunorubicin, doxorubicin, idarubicin, mithramycin, mitomycin, mitoxantrone, plicamycin and anthramycin (AMC); a toxin, such as a diphtheria toxin and active fragments thereof and hybrid molecules, a ricin toxin, cholera toxin, a Shiga-like toxin, LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins, momordica charantia inhibitor, curcin, crotin, gelonin, mitogillin, restrictocin, phenomycin, and enomycin toxins; or a pyrrolobenzodiazepine (PBD). PBD conjugates can include naturally occurring or synthetic PBDs. Naturally occurring PBDs include abbeymycin, anthramycin, chicamycin, DC-81, mazethramycin, neothramycins A and B, porothramycin, prothracarcin, sibanomicin (DC-102), sibiromycin, and tomamycin. Exemplary conjugates also include PBD dimers, including dimers containing a bridge that links the monomer PBD units of the dimer. The PBD dimer can be a homodimer or a heterodimer

In some examples, the antibody and targeted agent of the conjugate are linked directly. In other examples, the antibody and targeted agent of the conjugate are joined via a linker. The linker can be a peptide, a polypeptide or a chemical linker, which can be cleavable or non-cleavable. The linker can be conjugated to the antibody by several means. For example, the linker can be conjugated to one or more free thiols on the antibody, or to one or more primary amines on the antibody.

Also provided herein are nucleic acid molecule(s) that encode, such as the heavy chain of any of the anti-EGFR antibody, or antigen-binding fragment thereof, provided herein.

Provided are vectors that contain nucleic acid molecules that encode any of the anti-EGFR antibodies, EGFR-binding fragments, or heavy chains provided herein and cells, such as prokaryotic or eukaryotic cells that contain the vectors provided herein that contain nucleic acid molecules that encode any of the anti-EGFR antibodies, EGFR-binding fragments, or heavy chains provided herein.

Methods are also provided herein for making a modified anti-EGFR antibody, or antigen-binding fragment thereof, provided herein by expressing the heavy chain or light chain encoded from a vector or vectors provided herein encoding the heavy chain and the light chain in a suitable host cell and recovering the antibody.

Provided herein are combinations that include a modified anti-EGFR antibody or antigen-binding fragment provided herein, or a conjugate provided herein, and a chemotherapeutic agent or anti-cancer agent. The agent can be selected from among alkylating agents, nitrosoureas, topoisomerase inhibitors, and antibodies. In some examples, the chemotherapeutic agent is irinotecan, oxaliplatin, 5-fluorouracil (5-FU), Xeloda, Camptosar, Eloxatin, Adriamycin, paclitaxel, docetaxel, Cisplatin, gemcitabine or carboplatin. In some examples, a chemotherapeutic agent is an additional anti-EGFR antibody or antigen-binding fragment thereof that differs from the first antibody. In some examples, the additional anti-EGFR antibody is selected from among cetuximab, panitumumab, nimotuzumab, and antigen-binding fragments thereof or variants thereof

Provided herein are kits that include a modified anti-EGFR antibody or antigen-binding fragment provided herein, or a combination provided herein, in one or more containers, and instructions for use.

Provided herein are pharmaceutical compositions that include any of the modified anti-EGFR antibodies, antigen-binding fragments, or conjugates provided herein and a pharmaceutically acceptable carrier or excipient. A pharmaceutical composition provided herein can be formulated as a gel, ointment, liquid, suspension, aerosol, tablet, pill, powder or lyophile, and/or can be formulated for systemic, parenteral, topical, oral, mucosal, intranasal, subcutaneous, aerosolized, intravenous, bronchial, pulmonary, vaginal, vulvovaginal, esophageal, or oroesophageal administration. A pharmaceutical composition provided herein can be formulated for single dosage administration or for multiple dosage administration. In some examples, a pharmaceutical composition provided herein is a sustained release formulation.

Provided herein are methods of treating a condition responsive to treatment with an anti-EGFR antibody in a subject, including administering to the subject a pharmaceutically effective amount of a pharmaceutical composition provided herein. Examples of conditions that are responsive to treatment with an anti-EGFR antibody include a tumor, such as a solid tumor, cancer or metastasis, particularly when the tumor expresses EGFR.

In some examples, the condition responsive to treatment with an anti-EGFR antibody is head and neck cancer, non-small cell lung cancer or colorectal cancer. In some examples, a subject to be treated has a tumor that does not have a marker, such as KRAS, NRAS or BRAF, that confers resistance to anti-EGFR therapy. Thus in some examples, a subject can have a KRAS mutation-negative epidermal growth factor receptor (EGFR)-expressing colorectal cancer.

The subject for treatment can be a mammal, such as a human. The subject can be treated by topical, parenteral, local, or systemic administration of a pharmaceutical composition provided herein. For example, the pharmaceutical composition can be administered intranasally, intramuscularly, intradermally, intraperitoneally, intravenously, subcutaneously, orally, or by pulmonary administration.

The methods of treating a condition responsive to treatment with an anti-EGFR antibody in a subject provided herein can also include administration of one or more anticancer agents or treatments, such as irinotecan, oxaliplatin, 5-fluorouracil (5-FU), Xeloda, Camptosar, Eloxatin, Adriamycin, paclitaxel, docetaxel, Cisplatin, gemcitabine, carboplatin and radiation, or include administration of one or more additional anti-EGFR antibodies or antigen-binding fragments thereof, such as cetuximab, panitumumab, nimotuzumab, and antigen-binding fragments thereof.

In such methods, the pharmaceutical composition and the anticancer agent can be formulated as a single composition or as separate compositions, and the pharmaceutical composition and the anticancer agent can be administered sequentially, simultaneously or intermittently.

In the methods provided herein, the antibody can be administered at a dosage of about or 0.1 mg/kg to about or 100 mg/kg, such as, for example, about or 0.5 mg/kg to about or 50 mg/kg, about or 5 mg/kg to about or 50 mg/kg, about or 1 mg/kg to about or 20 mg/kg, about or 1 mg/kg to about or 100 mg/kg, about or 10 mg/kg to about or 80 mg/kg, or about or 50 mg/kg to about or 100 mg/kg or more; or at a dosage of about or 0.01 mg/m² to about or 800 mg/m² or more, such as for example, about or 0.01 mg/m², about or 0.1 mg/m², about or 0.5 mg/m², about or 1 mg/m², about or 5 mg/m², about or 10 mg/m², about or 15 mg/m², about or 20 mg/m², about or 25 mg/m², about or 30 mg/m², about or 35 mg/m², about or 40 mg/m², about or 45 mg/m², about or 50 mg/m², about or 100 mg/m², about or 150 mg/m², about or 200 mg/m², about or 250 mg/m², about or 300 mg/m², about or 400 mg/m², about or 500 mg/m², about or 600 mg/m² about or 700 mg/m².

Also provided herein are pharmaceutical compositions that can be formulated as a medicament for treating a condition responsive to treatment with an anti-EGFR antibody in a subject, and uses of pharmaceutical compositions for treating a condition responsive to treatment with an anti-EGFR antibody in a subject. Such pharmaceutical compositions or uses can be applied to a tumor, such as a tumor that is a solid tumor and/or expresses EGFR, cancer or metastasis. In particular examples, the condition to be treated by the pharmaceutical composition or use provided herein is head and neck cancer, non-small cell lung cancer or colorectal cancer.

### BRIEF DESCRIPTION OF THE FIGURES

FIG. 1 (A-B) depicts alignments of exemplary heavy and light chains of cetuximab in the art. For example, FIG. 1A depicts alignment of: the heavy chain amino acid sequence set forth in SEQ ID NO: 5, which contains a heavy chain variable domain (V_{H}) set forth in SEQ ID NO: 2 and the heavy chain constant domain (C_{H}) set forth in SEQ ID NO: 21; the heavy chain sequence set forth in SEQ ID NO: 6, which contains a V_{H} set forth in SEQ ID NO: 7 and a C_{H} set forth in SEQ ID NO: 22; the heavy chain sequence set forth in SEQ ID NO: 12, which contain the V_{H} set forth in SEQ ID NO: 2 and the C_{H} set forth in SEQ ID NO: 23; and the heavy chain sequence set forth in SEQ ID NO: 1, which contains the V_{H} set forth in SEQ ID NO: 2 and the C_{H} set forth in SEQ ID NO: 20. The heavy chain variable domain (V_{H}), three complementarity determining regions (CDRs) of the heavy chain (V_{H} CDR 1, V_{H} CDR 2, and V_{H} CDR 3), the three subdomains of the heavy chain constant domain (C_{H}1, C_{H}2, and C_{H}3) and the hinge region residues are indicated by arrows labeled with each of the regions or domains. FIG. 1B depicts the alignment of: the light chain sequence set forth in SEQ ID NO: 3, which contains the light chain variable domain (V_{L}) set forth in SEQ ID NO: 4 and the light chain constant domain (C_{L}) set forth in SEQ ID NO: 33; the light chain sequence set forth in SEQ ID NO: 10, which contains the V_{L} set forth in SEQ ID NO: 11 and the C_{L} set forth in SEQ ID NO: 33; the light chain sequence set forth in SEQ ID NO: 13, which contains the V_{L} set forth in SEQ ID NO: 4 and the C_{L} set forth in SEQ ID NO: 34; and the light chain sequence set forth in SEQ ID NO: 8, which c ntains the V_{L} set forth in SEQ ID NO: 9 and the C_{L} set forth in SEQ ID NO: 34. The light chain variable domain (V_{L}), three complementarity determining regions (CDRs) of the light chain (V_{L} CDR 1, V_{L} CDR 2, and V_{L} CDR 3), and the light chain constant domain (C_{L}) are indicated by arrows labeled with each of the regions or domains. In the depicted alignments, a “*” means that the aligned residues are identical, a “:” means that aligned residues are not identical, but are similar and contain conservative amino acids residues at the aligned position, and a “.” means that the aligned residues are similar and contain semi-conservative amino acid residues at the aligned position. The exemplary, non-limiting, position for amino acid replacements corresponding to position 104 is indicated by highlighting.

FIG. 2 (A-D) depicts alignments to identify corresponding residues between and among aligned antibodies. For example, FIG. 2A depicts the alignment of the heavy chain variable domains set forth in SEQ ID NO: 2 and 7 with the heavy chain variable domain of an exemplary unmodified anti-EGFR antibody designated H225 set forth in SEQ ID NO: 14. FIG. 2B depicts the alignment of the heavy chain variable domains set forth in SEQ ID NO: 2 and 7 with the heavy chain variable domain of an exemplary unmodified anti-EGFR antibody designated Hu225 set forth in SEQ ID NO: 16. The three subdomains of the heavy chain constant domain (C_{H}1, C_{H}2, and C_{H}3) are indicated by arrows labeled with each of the domains. FIG. 2C depicts the alignment of the light chain variable domains set forth in SEQ ID NO: 4, 9 and 11 with the light chain variable domain of an exemplary unmodified anti-EGFR antibody designated H225 set forth in SEQ ID NO: 15. FIG. 2D depicts the alignment of the light chain variable domains set forth in SEQ ID NO: 4, 9 and 11, to with the light chain variable domain of the exemplary unmodified anti-EGFR antibody designated Hu225 set forth in SEQ ID NO: 17. The three complementarity determining regions (CDRs) of the light chain (V_{L} CDR 1, V_{L} CDR 2, and V_{L} CDR 3) are indicated by arrows labeled with each of the domains. In the depicted alignments, a “*” means that the aligned residues are identical, a “:” means that aligned residues are not identical, but are similar and contain conservative amino acids residues at the aligned position, and a “.” means that the aligned residues are similar and contain semi-conservative amino acid residues at the aligned position. Exemplary, non-limiting, corresponding positions for amino acid replacements are indicated by highlighting.

### DETAILED DESCRIPTION

### Outline

A. DEFINITIONS
B. EGFR AND ANTI-EGFR ANTIBODIES
   1. EGFR
   2. Anti-EGFR antibodies and side effects
   3. Cetuximab (Erbitux) and derivatives thereof
      a. Structure
      b. Function
C. MODIFIED ACTIVE ANTI-EGFR ANTIBODIES WITH ACIDIC pH SELECTIVITY
   1. Anti-EGFR antibodies containing 104E modification
      a. Additional Modifications
         i. Additional heavy chain modifications
         ii. Additional light chain modifications
         iii. Other modifications
      b. Exemplary 104E modified anti-EGFR antibodies and fragments thereof
   2. Humanized anti-EGFR antibodies
   3. Anti-EGFR antibodies containing 104D modification
   4. Conjugates
      a. Targeted Agents
         i. Maytansinoid drug moieties
         ii. Auristatins and dolastatins drug moieties
         iii. Pyrrolobenzodiazepines (PBDs)
         iv. Cell toxin moieties
         v. Nucleic acids for targeted delivery
      b. Linkers
         i. Peptide linkers
         ii. Chemical linkers
      c. Exemplary Conjugates
         i. Anti-EGFR Antibody-Auristatin Conjugates
         ii. Anti-EGFR Antibody-Maytansinoid Conjugates
D. METHODS OF PRODUCING ANTI-EGFR ANTIBODIES
   1. Generating and producing anti-EGFR antibodies
      a. Vectors
      b. Cells and expression systems
         i. Prokaryotic expression
         ii. Yeast
         iii. Insects
         iv. Mammalian cells
         v. Plants
   2. Purification
E. METHODS FOR IDENTIFYING AND ASSESSING ANTI-EGFR ANTIBODY PROPERTIES AND ACTIVITIES
   1. Binding assays
   2. Cell based assays
   3. Animal models
   4. Pharmacokinetics and pharmacodynamics assays
F. PHARMACEUTICAL COMPOSITIONS, FORMULATIONS, KITS, ARTICLES OF MANUFACTURE AND COMBINATIONS
   1. Pharmaceutical compositions and formulations
   2. Articles of manufacture/kits
   3. Combinations
G. THERAPEUTIC USES
1. Exemplary diseases and conditions
   a. Cancer
   b. Non-cancer hyperproliferative diseases
   c. Autoimmune diseases or disorders
   d. Inflammatory disorders
   e. Infectious diseases
   f. Other diseases and conditions
   2. Subjects for therapy
      a. Selection of subjects overexpressing EGFR
      b. Selection of subjects exhibiting EGFR-associated polymorphism
      c. Identifying subjects exhibiting Anti-EGFR-associated side effects
         i. Skin toxicities
         ii. Hypomagnesemia
      d. Other methods of selecting or identifying subjects for treatment
   3. Dosages
   4. Routes of administration
   5. Combination therapies
H. EXAMPLES

### A. DEFINITIONS

Unless defined otherwise, all technical and scientific terms used herein have the same meaning as is commonly understood by one of skill in the art to which the invention(s) belong. All patents, patent applications, published applications and publications, GenBank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

As used herein, a conditionally active protein (e.g., antibody) is more active in one environment, particularly one in vivo environment, compared to a second environment. Hence, a conditionally active protein exhibits selective activity (e.g., binding activity) in one environment compared to another environment. For purposes herein, a conditionally active protein exhibits pH-selective activity, and is more active, under conditions that include one or both of pH 6.0 to 6.5, inclusive, and/or 10 mM to 20 mM lactate, inclusive, such as exists in a tumor environment, than under conditions that include one or both of pH of 7.0 to 7.4, inclusive, and/or 0.5 mM to 5 mM lactate (e.g., 1 mM), inclusive such as exists in a non-tumor environment, such as in the skin, GI tract or other non-tumor environment. Therefore, a conditionally active protein provided herein is a protein that exhibits selective activity, and is more active, in a tumor microenvironment than in a non-tumor microenvironment, such as the skin, GI tract or other non-tumor environment. Conditional activity can be manifested in vivo or in vitro. For example, conditional activity exists in vivo if the activity (e.g., binding activity) in a tumor environment is greater than a non-tumor environment, for example the ratio of activity in the tumor environment compared to the non-tumor microenvironment is at least 1.1, 1.2, 1.3, 1.4, 1.5, 1.6, 1.7, 1.8, 1.9, 2.0, 2.5, 3.0, 3.5, 4.0, 4.5, 5.0, 6.0, 7.0, 8.0, 9.0, 10.0, 11.0, 12.0, 13.0, 14.0, 15.0, 16.0, 17.0, 18.0, 19.0, 20.0, 25.0, 30.0, 35.0, 40.0, 45.0, 50.0 or more.

As used herein, a therapeutic agent that has “conditional activity in a tumor microenvironment,” or is “conditionally active in a tumor microenvironment,” or variations thereof, is a therapeutic agent, such as a modified anti-EGFR antibody provided herein, that is more active as a therapeutic in a tumor microenvironment than in a non-tumor microenvironment (e.g., a healthy or non-diseased tissue or cell, such as the basal layer of the skin).

As used herein, “pH-selective activity” refers to a protein (e.g., an antibody) that is more active under conditions that include, or in the presence of, acidic pH (e.g., pH 6.0 to 6.5, and optionally elevated lactate levels, e.g., 10 mM to 20 mM) than in an environment of neutral pH (e.g., pH 7.0 to 7.4, and optionally normal lactate concentrations, e.g., 0.5 mM to 5 mM). pH-selective activity can be manifested in vivo or in vitro. pH-selective activity exists if the activity (e.g., binding activity) is greater under acidic conditions (e.g., pH 6.0 to 6.5 and/or 10 mM to 20 mM lactate) than under neutral conditions (e.g., pH 7.0 to 7.4 and/or 0.5 mM to 5 mM lactate). For example, pH-selective activity exists if the ratio of activity under acidic conditions to neutral conditions is at least 1.1, 1.2, 1.3, 1.4, 1.5, 1.6, 1.7, 1.8, 1.9, 2.0, 2.5, 3.0, 3.5, 4.0, 4.5, 5.0, 6.0, 7.0, 8.0, 9.0, 10.0, 11.0, 12.0, 13.0, 14.0, 15.0, 16.0, 17.0, 18.0, 19.0, 20.0, 25.0, 30.0, 35.0, 40.0, 45.0, 50.0 or more.

As used herein, “conditions that simulate” a diseased or non-diseased microenvironment, refer to in vitro or in vivo assay conditions that correspond to a condition or conditions that exist in the environment in vivo. For example, if a microenvironment is characterized by low or acidic pH, then conditions that simulate the microenvironment include buffer or assay conditions have a low or acidic pH.

As used herein, conditions that exist in a tumor microenvironment include conditions that exist therein compared to a non-tumor microenvironment (e.g., a healthy or non-diseased cell or tissue). Conditions that exist in a tumor microenvironment include increased vascularization, hypoxia, low pH, increased lactate concentration, increased pyruvate concentration, increased interstitial fluid pressure and altered metabolites or metabolism indicative of a tumor. For example, a condition that exists in a tumor microenvironment is low pH, i.e., pH less than 7.4, typically between or about between 5.6 to 6.8, such as less than or about or pH 5.6, 5.7, 5.8, 5.9, 6.0, 6.1, 6.2, 6.3, 6.4, 6.5, 6.6, 6.7, or 6.8. A condition that exists in a tumor microenvironment also can include a high lactate concentration at or about between 5 mM to 20 mM lactic acid, for example 10 mM to 20 mM lactic acid such as 15 mM to 18 mM, and in particular at least or at least about or 16 mM, 16.5 mM, 16.7 mM or 17 mM lactic acid.

As used herein, conditions that exist in a non-tumor microenvironment include a condition or conditions that are not present in a tumor microenvironment. For purposes herein, the conditions or condition is the corresponding property or characteristic that is present in a tumor microenvironment and non-tumor environment, such as pH, lactate concentration or pyruvate concentration, but that differs between the two microenvironments. A condition that exists in a non-tumor microenvironment (e.g., basal layer of the skin) is a pH from about 7.0 to about 7.8, such as at least or about or pH 7.1, 7.2, 7.3, 7.4, 7.5, 7.6, 7.7 or 7.8. For example, the pH is a neutral pH of between or about between 7.0 to 7.4, such as or about pH 7.4. A condition that exists in a non-tumor microenvironment (e.g., basal layer of the skin) also includes a lactate concentration that is 0.5 to 5 mM lactate, such as, for example 0.5 mM to 4 mM lactic acid, for example about or 0.5, 1, 2, 3, 4, or 5 mM lactic acid.

As used herein, “low pH” or “acidic pH”, which are used interchangeably herein, refers to a pH ranging from about 5.6 to about 6.8, such as less than or about or pH 5.6, 5.7, 5.8, 5.9, 6.0, 6.1, 6.2, 6.3, 6.4, 6.5, 6.6, 6.7, or 6.8. For example, a low pH or acidic pH is between 6.0 to 6.5, inclusive, such as or about pH 6.0 or pH 6.5.

As used herein, epidermal growth factor receptor (EGFR; Uniprot Accession No. P00533 and set forth in SEQ ID NO: 43) refers to a tyrosine kinase growth factor receptor that is a member of the ErbB family of receptor tyrosine kinases and that is bound and activated by ligands such as epidermal growth factor (EGF), as well as other endogenous EGF-like ligands including TGF-α, amphiregulin, heparin-binding EGF (HB-EGF) and betacellulin. Upon activation, EGFR is involved in signaling cascades important for cell growth, proliferation, survival and motility. In addition to their presence on a tumor cells, epidermal growth factor receptors are ubiquitous, distributed randomly on the surface of normal cells, excluding hematopoietic cells and cells of epidermal origin. For example, EGFR is expressed on skin keratinocytes.

As used herein, ratio of activity with reference to binding activity of a modified anti-EGFR antibody or antigen-binding fragment thereof refers to the relation of binding activity to EGFR antigen (e.g., human EGFR or soluble fragment thereof) under a first set of conditions that include one or both of pH 6.0 to 6.5, inclusive, and lactate concentration between 15 mM to 20 mM, inclusive, compared to under a second set of conditions that include one or both of pH about or 7.4 and lactate concentration of about or 1 mM. It is expressed by the quotient of the division of the activity at the first condition by the activity at the second condition, as long as the activity positively correlates with the binding activity. In some instances herein, binding activity is provided as a measure that negatively correlates with binding activity (e.g., EC50 or KD). In such examples, the ration of activity is expressed first as the inverse of the binding activity under both set of conditions, and then as the quotient of the division of the inverse of the activity at the first condition by the activity at the second condition. It is understood that in determining binding activity and the ratio of binding activity, the binding activity under the first and second condition is measured under the same assay conditions, except for the difference in pH and/or lactic acid concentration. A ratio of binding activity of >1 indicates that binding activity is greater or higher under the first set of conditions than under the second set of conditions.

As used herein, anti-EGFR antibody refers to any antibody that specifically binds to epidermal growth factor receptor (EGFR) or a soluble fragment thereof and blocks the binding of ligands to EGFR, thereby resulting in competitive inhibition of EGFR and inhibition of EGFR activation. Hence, anti-EGFR antibodies are EGFR inhibitors. Reference to anti-EGFR antibodies herein include a full-length antibody and antigen-binding fragments thereof that specifically bind to EGFR.

As used herein, an epidermal growth factor receptor (EGFR) antigen refers to a tyrosine growth factor receptor that is bound by ligands such as epidermal growth factor (EGF). EGFR includes human and non-human proteins. In particular, EGFR antigen includes human EGFR, which is a 170 kDa Type I glycoprotein that has the sequence of amino acids set forth in SEQ ID NO: 43 (see e.g., Uniprot Accession No. P00533).

As used herein, a soluble EGFR refers to soluble EGFR isoforms (sEGFR) that lack the transmembrane or intracellular domain. Hence, a soluble EGFR includes proteins that include only the extracellular domain (ECD) portion of EGFR. An exemplary soluble EGFR contains only the ECD of EGFR set forth in SEQ ID NO: 43 or a portion thereof sufficient to bind EGF, corresponding to amino acid residues 25-645 of SEQ ID NO: 43 or a portion thereof sufficient to bind EGF. A soluble EGFR also can include proteins that are linked, directly or indirectly, to other domains or regions of other proteins.

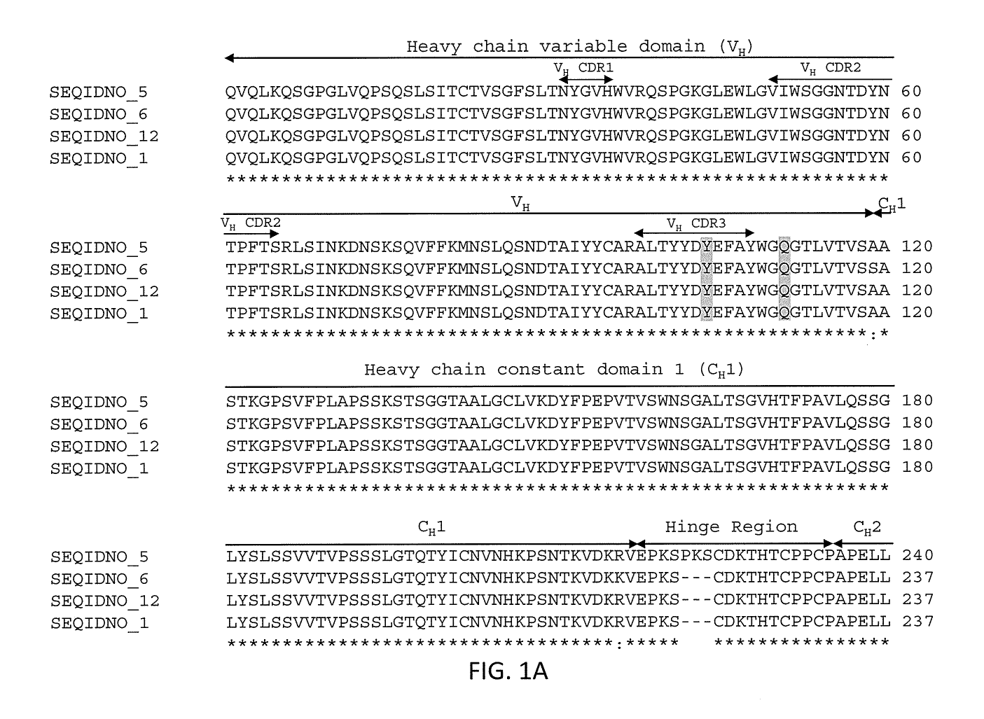

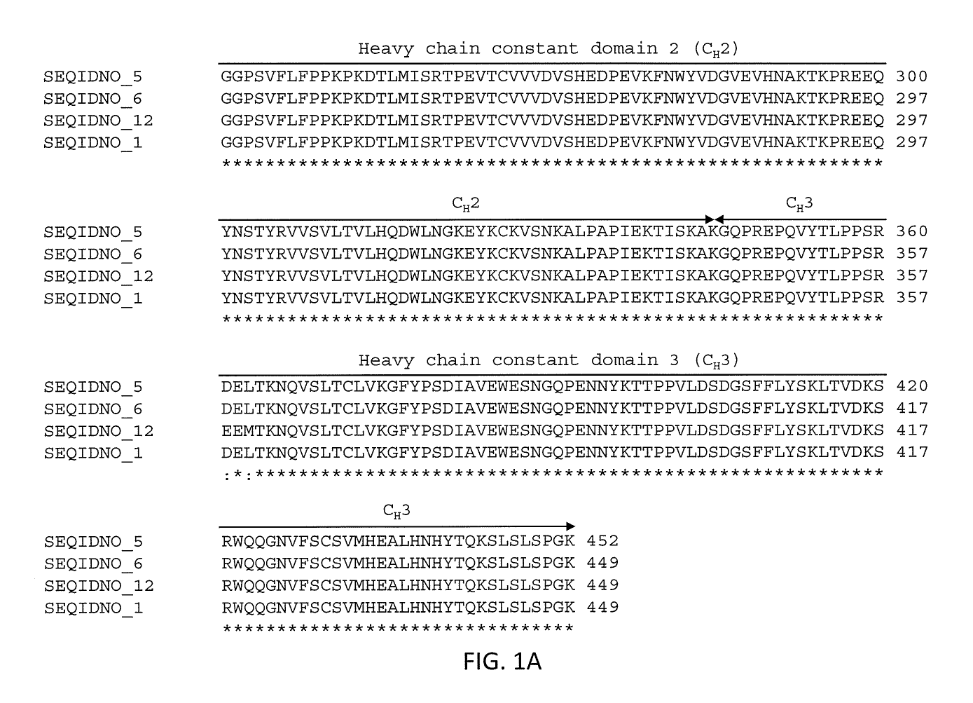

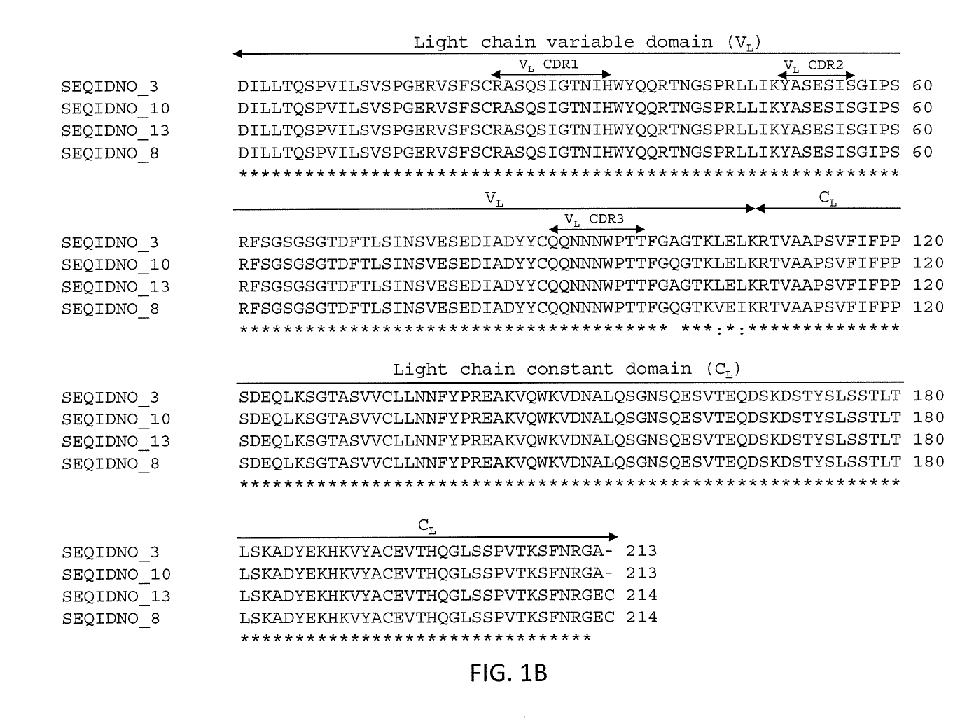

As used herein, cetuximab (225, also known and marketed as Erbitux) refers to an anti-EGFR antibody that is a chimeric (mouse/human) monoclonal antibody that specifically binds EGFR and is an EGFR inhibitor. Cetuximab is reported to be composed of 4 polypeptide chains, including 2 identical heavy chains of 449 amino acids each (e.g., set forth in SEQ ID NO: 12), and 2 identical light chains of 214 amino acids each (e.g., set forth in SEQ ID NO: 13) (see IMGT Acc. No. 7906). The variable regions corresponding to the variable regions of M225 are set forth as amino acid residues 1-119 of SEQ ID NO: 12 (variable heavy chain, set forth in SEQ ID NO: 2) and as amino acid residues 1-107 of SEQ ID NO: 13 (variable light chain, set forth as SEQ ID NO: 4). C225 contains a human IgG1 heavy chain constant region set forth as amino acid residues 120-449 of SEQ ID NO: 12 (set forth in SEQ ID NO: 23) containing human constant domains C_{H}1-C_{H}2-hinge-C_{H}3, including C_{H}1 (amino acid residues 120-217 of SEQ ID NO: 12), a hinge region (amino acid residues 218-232 of SEQ ID NO: 12), C_{H}2 (amino acid residues 233-342 of SEQ ID NO: 12) and C_{H}3 (amino acid residues 343-449 of SEQ ID NO: 12). C225 also contains a human Cκ light chain constant region set forth as amino acid residues 108-213 of SEQ ID NO: 13 (set forth as SEQ ID NO: 34). Reference to cetuximab herein also refers to antibodies reported in the literature that that differ by only a few amino acids (see, e.g., U.S. Pat. No. 7,060,808; published U.S. Patent Appl. No. US 20110117110; U.S. Patent Publ. No. US 20130266579; International Published PCT Appl. No. WO2004085474; GenBank Accession No. CAH61633; DrugBank Acc. No. DB00002; IMGT Acc. No. 7906). Thus, reference to cetuximab herein also includes the sequence of amino acids set forth in SEQ ID NOS: 1 (heavy chain) and 3 (light chain); SEQ ID NOS: 5 (heavy chain) and 3 (light chain); SEQ ID NOS: 6 (heavy chain) and 8 (light chain); or SEQ ID NOS: 6 (heavy chain) and 10 (light chain). The cetuximab sequences and corresponding SEQ ID NOS are provided in FIGS. 1A and 1B and in Table 5.

Reference to cetuximab herein, when so indicated, also includes humanized or other variant derivatives of cetuximab that contain complementarity determining regions (CDRs) identical to cetuximab. The CDRs of cetuximab include, V_{H} CDR 1 (amino acid residues to 31-35, according to Kabat definition, of SEQ ID NO: 2 or 7, set forth in SEQ ID NO: 35); V_{H} CDR 2 (amino acid residues 50-65 of SEQ ID NO: 2 or 7, set forth in SEQ ID NO: 36); V_{H} CDR 3 (amino acid residues 98-108 of SEQ ID NO: 2 or 7, set forth in SEQ ID NO: 37); V_{L} CDR 1 (amino acid residues 24-34 of SEQ ID NO: 4, 9 or 11, set forth in SEQ ID NO: 38); V_{L} CDR 2 (amino acid residues 50-56 of SEQ ID NO: 4, 9 or 11, set forth in SEQ ID NO: 39); and V_{L} CDR 3 (amino acid residues 89-97 of SEQ ID NO: 4, 9 or 11, set forth in SEQ ID NO: 40), see, e.g., U.S. Publ. No. US 20110117110.

As used herein, an antigen-binding fragment of cetuximab refers to an antibody derived from cetuximab but that is less than the full length of cetuximab but contains at least a portion of the variable region of the antibody sufficient to form an antigen binding site (e.g., one or more CDRs) and thus retains the binding specificity and/or activity of cetuximab. The variable region of the cetuximab heavy chain is set forth in SEQ ID NO: 2 or 7, which corresponds to amino acids 1-119 of SEQ ID NO: 1, 5, 6, or 12. The variable region of the cetuximab light chain is set forth in SEQ ID NO: 4, 9, or 11, which corresponds to amino acids 1-107 of SEQ ID NO: 3, 8, 10 or 13 (see FIG. 1A or 1B and Table 5). Thus, exemplary antigen-binding fragments of cetuximab include antibodies that contain the sequence of amino acids set forth in SEQ ID NO: 2 (variable heavy chain) and the sequence of amino acids set forth in SEQ ID NO: 4 (variable light chain), antibodies that contain the sequence of amino acids set forth in SEQ ID NO: 7 (variable heavy chain) and the sequence of amino acids set forth in SEQ ID NO: 9 (variable light chain), antibodies that contain the sequence of amino acids set forth in SEQ ID NO: 7 (variable heavy chain) and the sequence of amino acids set forth in SEQ ID NO: 11 (variable light chain) or a portion of the variable heavy or light chain sufficient to bind to antigen. For example, an exemplary antigen-binding fragment of cetuximab is a Fab antibody that contains the sequence of amino acids set forth in SEQ ID NO: 2 or 7 and that includes a CH1 region of an IgG1 antibody set forth in any of SEQ ID NOS: 23 or CH1 region of other reported IgG1 set forth in any of SEQ ID NOS: 19-22 (VH-CH1) and SEQ ID NO: 3 (light chain VH-CL).

As used herein, an “unmodified antibody” refers to a starting polypeptide heavy and light chain or fragment thereof that is selected for modification as provided herein. The starting target polypeptide can be a wild-type or reference form of an antibody, which is a predominant reference polypeptide to which activity is assessed. For example, cetuximab is a predominant or reference polypeptide for modification herein. The unmodified or starting target antibody can be altered or mutated, such that it differs from a predominant or reference form of the antibody, but is nonetheless referred to herein as a starting unmodified target protein relative to the subsequently modified polypeptides produced herein (e.g., antigen-binding fragments or variants of cetuximab). Thus, existing proteins known in the art that have been modified to have a desired increase or decrease in a particular activity or property compared to an unmodified reference protein can be selected and used as the starting unmodified target protein.

For example, a protein that has been modified from a predominant or reference form by one or more single amino acid changes and possesses either an increase or decrease in a desired property, such as reduced immunogenicity, can be a target protein, referred to herein as unmodified, for further modification of either the same or a different property. Exemplary reference or unmodified anti-EGFR antibodies are full length anti-EGFR antibody polypeptides set forth in SEQ ID NOS: 1 (Heavy Chain) and 3 (Light Chain), SEQ ID NOS: 5 (Heavy Chain) and 3 (Light Chain), SEQ ID NOS: 12 (Heavy Chain) and 13 (Light Chain), or SEQ ID NOS: 6 (Heavy Chain) and 8 (Light Chain), SEQ ID NOS: 6 (Heavy Chain) and 10 (Light Chain); or antigen-binding fragments thereof. Exemplary antigen-binding fragments include anti-EGFR antibody fragments that contain the polypeptide set forth in SEQ ID NOS: 2 (variable Heavy Chain) and 4 (variable light chain), SEQ ID NOS: 7 (variable Heavy Chain) and 9 (variable light chain), or SEQ ID NO: 7 (variable heavy chain) and SEQ ID NO: 11 (variable light chain). An unmodified or reference antibody also includes antibody variants thereof that exhibit heavy or light chains or portions thereof that exhibit at least 68%, 69%, 70%, 75%, 80%, 85%, 86%, 87%, 88%, 89%, 90%, 91%, 92%, 93%, 94%, 95%, 96%, 97%, 98%, or 99% sequence identity thereto to any of the recited SEQ ID NOS, whereby the resulting antibody specifically binds EGFR.

As used herein, “modified anti-EGFR antibody” or “variant anti-EGFR antibody” refers to an anti-EGFR antibody that contains at least one amino acid addition, deletion or replacement as described herein in its sequence of amino acids compared to a reference or unmodified anti-EGFR antibody. For purposes herein, the at least one amino acid replacement is replacement with glutamic acid (E) in the variable heavy chain at a position corresponding to position 104 with reference to SEQ ID NO: 2 or 7. A modified anti-EGFR antibody can contain additional modifications (e.g., amino acid replacements). For example, a modified anti-EGFR antibody can have up to 150 amino acid replacements, as long as the resulting modified anti-EGFR antibody exhibits binding to EGFR. Typically, a modified anti-EGFR antibody contains 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid replacements compared to an unmodified antibody. It is understood that a modified anti-EGFR antibody also can include any one or more other modifications, in addition to at least one amino acid addition, deletion or replacement as described herein.

As used herein, “both” with reference to modifications in a variable heavy chain, variable light chain or both means that an antibody contains one or more modifications in the variable heavy chain and one or more modifications in the variable light chain of the antibody.

As used herein, a “modification” is in reference to modification of a sequence of amino acids of a polypeptide or a sequence of nucleotides in a nucleic acid molecule and includes deletions, insertions, and replacements of amino acids or nucleotides, respectively. Methods of modifying a polypeptide are routine to those of skill in the art, such as by using recombinant DNA methodologies.

As used herein, “deletion,” when referring to a nucleic acid or polypeptide sequence, refers to the deletion of one or more nucleotides or amino acids compared to a sequence, such as a target polynucleotide or polypeptide or a native or wild-type sequence.

As used herein, “insertion” when referring to a nucleic acid or amino acid sequence, describes the inclusion of one or more additional nucleotides or amino acids, within a target, native, wild-type or other related sequence. Thus, a nucleic acid molecule that contains one or more insertions compared to a wild-type sequence, contains one or more additional nucleotides within the linear length of the sequence. As used herein, “additions,” to nucleic acid and amino acid sequences describe addition of nucleotides or amino acids onto either termini compared to another sequence.

As used herein, “substitution” or “replacement” refers to the replacing of one or more nucleotides or amino acids in a native, target, wild-type or other nucleic acid or polypeptide sequence with an alternative nucleotide or amino acid, without changing the length (as described in numbers of residues) of the molecule. Thus, one or more substitutions in a molecule does not change the number of amino acid residues or nucleotides of the molecule. Amino acid replacements compared to a particular polypeptide can be expressed in terms of the number of the amino acid residue along the length of the polypeptide sequence. For example, a modified polypeptide having a modification in the amino acid at the 104^{th} position of the amino acid sequence that is a substitution/replacement of Tyrosine (Tyr; T) with glutamic acid (Glu; E) can be expressed as Y104E, Tyr104Glu, or 104E. Simply Y104 can be used to indicate that the amino acid at the modified 104^{th} position is a tyrosine. For purposes herein, since modifications are in a heavy chain (HC) or light chain (LC) of an antibody, modifications also can be denoted by reference to HC- or LC- to indicate the chain of the polypeptide that is altered.

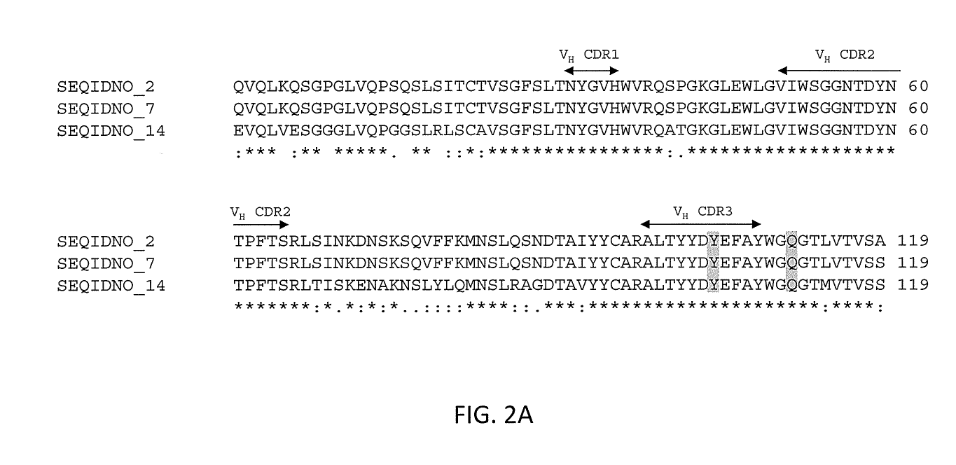

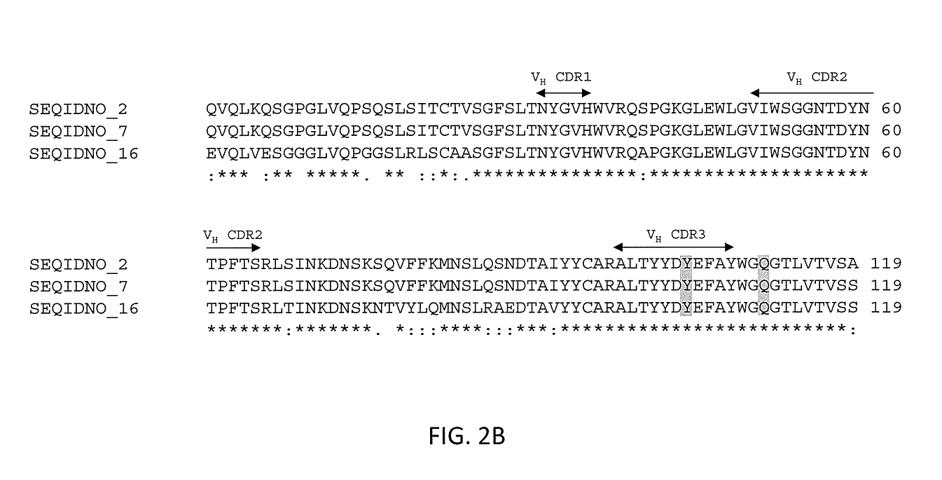

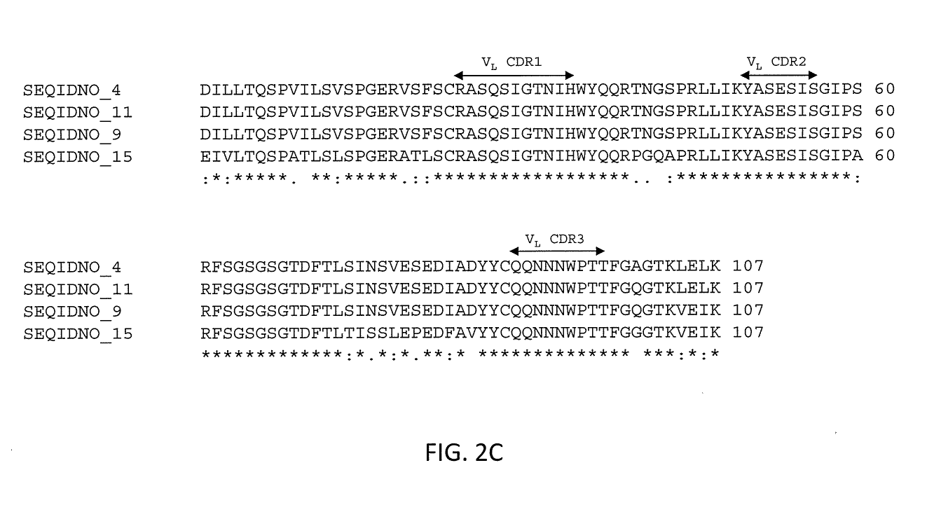

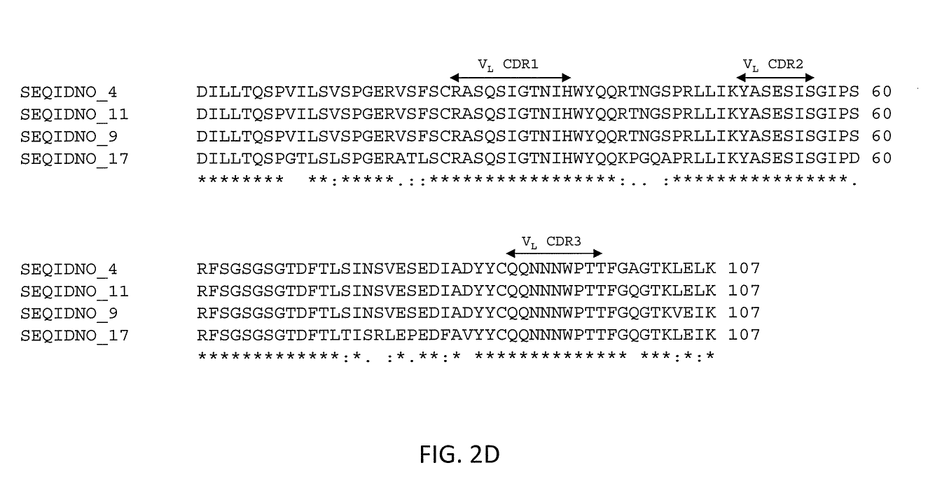

As used herein, “at a position corresponding to” or recitation that nucleotides or amino acid positions “correspond to” nucleotides or amino acid positions in a disclosed sequence, such as set forth in the Sequence Listing, refers to nucleotides or amino acid positions identified upon alignment with the disclosed sequence to maximize identity using a standard alignment algorithm, such as the GAP algorithm. For purposes herein, residues for modification provided herein are with reference to amino acid positions set forth in the variable heavy chain set forth in SEQ ID NO: 2 or 7 and the variable light chain set forth in SEQ ID NO: 4, 9 or 11. Hence, corresponding residues can be determined by alignment of a reference heavy chain sequence, or portion thereof, with the sequence set forth in SEQ ID NO: 2 or 7 (e.g., FIG. 2A or 2B) and/or by alignment of a reference light chain sequence, or portion thereof, with the sequence set forth in SEQ ID NO: 4, 9 or 11 (e.g., FIG. 2C or 2D). By aligning the sequences, one skilled in the art can identify corresponding residues, for example, using conserved and identical amino acid residues as guides. In general, to identify corresponding positions, the sequences of amino acids are aligned so that the highest order match is obtained (see, e.g., Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48:1073). Exemplary alignments are provided in FIG. 2A-D and exemplary amino acid replacements based on corresponding aligned residues are set forth in Table 6 and Table 8.

As used herein, alignment of a sequence refers to the use of homology to align two or more sequences of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence. Related or variant polypeptides or nucleic acid molecules can be aligned by any method known to those of skill in the art. Such methods typically maximize matches, and include methods, such as using manual alignments and by using the numerous alignment programs available (e.g., BLASTP) and others known to those of skill in the art. By aligning the sequences of polypeptides or nucleic acids, one skilled in the art can identify analogous portions or positions, using conserved and identical amino acid residues as guides. Further, one skilled in the art also can employ conserved amino acid or nucleotide residues as guides to find corresponding amino acid or nucleotide residues between and among human and non-human sequences. Corresponding positions also can be based on structural alignments, for example by using computer simulated alignments of protein structure. In other instances, corresponding regions can be identified. One skilled in the art also can employ conserved amino acid residues as guides to find corresponding amino acid residues between and among human and non-human sequences.

As used herein, recitation that proteins are “compared under the same conditions” means that different proteins are treated identically or substantially identically such that any one or more conditions that can influence the activity or properties of a protein or agent are not varied or not substantially varied between the test agents. For example, when the activity of a modified anti-EGFR antibody is compared to an unmodified anti-EGFR antibody any one or more conditions such as amount or concentration of the polypeptide; presence, including amount, of excipients, carriers or other components in a formulation other than the active agent (e.g., anti-EGFR antibody); temperature; pH; time of storage; storage vessel; properties of storage (e.g., agitation) and/or other conditions associated with exposure or use are identical or substantially identical between and among the compared polypeptides.

As used herein, an “adverse effect,” or “side effect” or “adverse event,” or “adverse side effect” refers to a harmful, deleterious and/or undesired effect associated with administering a therapeutic agent. For example, side effects associated with administration of an anti-EGFR antibody, such as cetuximab are known to one of skill in the art and described herein. Such side effects include, for example, dermatological or dermal toxicity such as rash. Side effects or adverse effects are graded on toxicity, and various toxicity scales exist providing definitions for each grade. Examples of such scales are toxicity scales of the National Cancer Institute Common Toxicity Criteria version 2.0, the World Health Organization or Common Terminology Criteria for Adverse Events (CTCAE) scale. Generally, the scale is as follows: Grade 1=mild side effects; Grade 2=moderate side effects; Grade 3=severe side effects; Grade 4=life threatening or disabling side-effects; Grade 5=fatal. Assigning grades of severity is within the skill of an experienced physician or other health care professional.

As used herein, a “property” of a polypeptide, such as an antibody, refers to any property exhibited by a polypeptide, including, but not limited to, binding specificity, structural configuration or conformation, protein stability, resistance to proteolysis, conformational stability, thermal tolerance, and tolerance to pH conditions. Changes in properties can alter an “activity” of the polypeptide. For example, a change in the binding specificity of the antibody polypeptide can alter the ability to bind an antigen, and/or various binding activities, such as affinity or avidity, or in vivo activities of the polypeptide.

As used herein, an “activity” or a “functional activity” of a polypeptide, such as an antibody, refers to any activity exhibited by the polypeptide. Such activities can be empirically determined Exemplary activities include, but are not limited to, ability to interact with a biomolecule, for example, through antigen-binding, DNA binding, ligand binding, or dimerization, enzymatic activity, for example, kinase activity or proteolytic activity. For an antibody (including antibody fragments), activities include, but are not limited to, the ability to specifically bind a particular antigen, affinity of antigen-binding (e.g., high or low affinity), avidity of antigen-binding (e.g., high or low avidity), on-rate, off-rate, effector functions, such as the ability to promote antigen neutralization or clearance, virus neutralization, and in vivo activities, such as the ability to prevent infection or invasion of a pathogen, or to promote clearance, or to penetrate a particular tissue or fluid or cell in the body. Activity can be assessed in vitro or in vivo using recognized assays, such as ELISA, flow cytometry, surface plasmon resonance or equivalent assays to measure on- or off-rate, immunohistochemistry and immunofluorescence histology and microscopy, cell-based assays, flow cytometry and binding assays (e.g., panning assays). For example, for an antibody polypeptide, activities can be assessed by measuring binding affinities, avidities, and/or binding coefficients (e.g., for on-/off-rates), and other activities in vitro or by measuring various effects in vivo, such as immune effects, e.g., antigen clearance; penetration or localization of the antibody into tissues; protection from disease, e.g., infection; serum or other fluid antibody titers; or other assays that are well-known in the art. The results of such assays that indicate that a polypeptide exhibits an activity can be correlated to activity of the polypeptide in vivo, in which in vivo activity can be referred to as therapeutic activity, or biological activity. Activity of a modified polypeptide can be any level of percentage of activity of the unmodified polypeptide, including but not limited to, 1% of the activity, 2%, 3%, 4%, 5%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more of activity compared to the unmodified polypeptide. Assays to determine functionality or activity of modified (or variant) antibodies are well-known in the art.

As used herein, “bind,” “bound” or grammatical variations thereof refers to the participation of a molecule in any attractive interaction with another molecule, resulting in a stable association in which the two molecules are in close proximity to one another. Binding includes, but is not limited to, non-covalent bonds, covalent bonds (such as reversible and irreversible covalent bonds), and includes interactions between molecules such as, but not limited to, proteins, nucleic acids, carbohydrates, lipids, and small molecules, such as chemical compounds including drugs. Exemplary bonds are antibody-antigen interactions and receptor-ligand interactions. When an antibody “binds” a particular antigen, bind refers to the specific recognition of the antigen by the antibody, through cognate antibody-antigen interaction, at antibody combining sites. Binding also can include association of multiple chains of a polypeptide, such as antibody chains which interact through disulfide bonds.

As used herein, binding activity refers to characteristics of a molecule, e.g., a polypeptide, relating to whether or not, and how, it binds one or more binding partners. Binding activities include the ability to bind the binding partner(s), the affinity with which it binds to the binding partner (e.g., high affinity), the avidity with which it binds to the binding partner, the strength of the bond with the binding partner and/or specificity for binding with the binding partner.

As used herein, “affinity” or “binding affinity” describes the strength of the interaction between two or more molecules, such as binding partners, typically the strength of the noncovalent interactions between two binding partners. The affinity of an antibody or antigen-binding fragment thereof for an antigen epitope is the measure of the strength of the total noncovalent interactions between a single antibody combining site and the epitope. Low-affinity antibody-antigen interaction is weak, and the molecules tend to dissociate rapidly, while high affinity antibody-antigen-binding is strong and the molecules remain bound for a longer amount of time. Binding affinity can be determined in terms of binding kinetics, such as measuring rates of association (kₐ or kₒₙ) and/or dissociation (k_{d} or k_{off}), half maximal effective concentration (EC₅₀) values, and/or thermodynamic data (e.g., Gibbs free energy (ΔG), enthalpy (ΔH), entropy (−TΔS), and/or calculating association (Kₐ) or dissociation (K_{d}) constants.

EC₅₀, also called the apparent K_{d}, is the concentration (e.g., ng/mL) of antibody, where 50% of the maximal binding is observed to a fixed amount of antigen. Typically, EC₅₀ values are determined from sigmoidal dose-response curves, where the EC₅₀ is the concentration at the inflection point. A high antibody affinity for its substrate correlates with a low EC₅₀ value and a low affinity corresponds to a high EC₅₀ value. Affinity constants can be determined by standard kinetic methodology for antibody reactions, for example, immunoassays, such as ELISA, followed by curve-fitting analysis.

As used herein, “affinity constant” refers to an association constant (Kₐ) used to measure the affinity of an antibody for an antigen. The higher the affinity constant the greater the affinity of the antibody for the antigen. Affinity constants are expressed in units of reciprocal molarity (i.e., M⁻¹) and can be calculated from the rate constant for the association-dissociation reaction as measured by standard kinetic methodology for antibody reactions (e.g., immunoassays, surface plasmon resonance, or other kinetic interaction assays known in the art). The binding affinity of an antibody also can be expressed as a dissociation constant, or K_{d}. The dissociation constant is the reciprocal of the association constant, K_{d}=1/Kₐ. Hence, an affinity constant also can be represented by the K_{d}. Affinity constants can be determined by standard kinetic methodology for antibody reactions, for example, immunoassays, surface plasmon resonance (SPR) (Rich and Myszka (2000) Curr. Opin. Biotechnol 11:54; Englebienne (1998) Analyst. 123:1599), isothermal titration calorimetry (ITC) or other kinetic interaction assays known in the art (see, e.g., Paul, ed., Fundamental Immunology, 2nd ed., Raven Press, New York, pages 332-336 (1989); see also U.S. Pat. No. 7,229,619 for a description of exemplary SPR and ITC methods for calculating the binding affinity of antibodies). Instrumentation and methods for real time detection and monitoring of binding rates are known and are commercially available (e.g., BIAcore 2000, BIAcore AB, Upsala, Sweden and GE Healthcare Life Sciences; Malmqvist (2000) Biochem. Soc. Trans. 27:335).

Methods for calculating affinity are well-known, such as methods for determining EC₅₀ values or methods for determining association/dissociation constants. For example, in terms of EC₅₀, high binding affinity means that the antibody specifically binds to a target protein with an EC₅₀ that is less than about 10 ng/mL, 9 ng/mL, 8 ng/mL, 7 ng/mL, 6 ng/mL, 5 ng/mL, 3 ng/mL, 2 ng/mL, 1 ng/mL or less. High binding affinity also can be characterized by an equilibrium dissociation constant (K_{d}) of 10⁻⁶ M or lower, such as 10⁻⁷ M, 10⁻⁸ M, 10⁻¹⁰ M, 10⁻¹¹ M or 10⁻¹² M or lower. In terms of equilibrium association constant (Kₐ), high binding affinity is generally associated with Kₐ values of greater than or equal to about 10⁶ M⁻¹, greater than or equal to about 10⁷M⁻¹, greater than or equal to about 10⁸ M⁻¹, or greater than or equal to about 10⁹ M⁻¹, 10¹⁰ M⁻¹, 10¹¹ M⁻¹ or 10¹² M⁻¹. Affinity can be estimated empirically or affinities can be determined comparatively, e.g., by comparing the affinity of two or more antibodies for a particular antigen, for example, by calculating pairwise ratios of the affinities of the antibodies tested. For example, such affinities can be readily determined using conventional techniques, such as by ELISA; equilibrium dialysis; surface plasmon resonance; by radioimmunoassay using radiolabeled target antigen; or by another method known to the skilled artisan. The affinity data can be analyzed, for example, by the method of Scatchard et al., Ann N.Y. Acad. Sci., 51:660 (1949) or by curve fitting analysis, for example, using a 4 Parameter Logistic nonlinear regression model using the equation: y=((A−D)/(1+((x/C)̂B)))+D, where A is the minimum asymptote, B is the slope factor, C is the inflection point (EC₅₀), and D is the maximum asymptote.

As used herein, antibody avidity refers to the strength of multiple interactions between a multivalent antibody and its cognate antigen, such as with antibodies containing multiple binding sites associated with an antigen with repeating epitopes or an epitope array. A high avidity antibody has a higher strength of such interactions compared to a low avidity antibody.

As used herein, “exhibits at least one activity” or “retains at least one activity” refers to the activity exhibited by a modified polypeptide, such as a variant antibody or other therapeutic polypeptide (e.g., a modified anti-EGFR antibody or antigen-binding fragment thereof), compared to the target or unmodified polypeptide, that does not contain the modification. A modified, or variant, polypeptide that retains an activity of a target polypeptide can exhibit improved activity, decreased activity, or maintain the activity of the unmodified polypeptide. In some instances, a modified, or variant, polypeptide can retain an activity that is increased compared to a target or unmodified polypeptide. In some cases, a modified, or variant, polypeptide can retain an activity that is decreased compared to an unmodified or target polypeptide. Activity of a modified, or variant, polypeptide can be any level of percentage of activity of the unmodified or target polypeptide, including but not limited to, 1% of the activity, 2%, 3%, 4%, 5%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more activity compared to the unmodified or target polypeptide. In other embodiments, the change in activity is at least about 2 times, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more times greater than unmodified or target polypeptide. Assays for retention of an activity depend on the activity to be retained. Such assays can be performed in vitro or in vivo. Activity can be measured, for example, using assays known in the art and described in the Examples below for activities, such as, but not limited to, ELISA and panning assays. Activities of a modified, or variant, polypeptide compared to an unmodified or target polypeptide also can be assessed in terms of an in vivo therapeutic or biological activity or result following administration of the polypeptide.

As used herein, “increased activity” with reference to a modified anti-EGFR antibody means that, when tested under the same conditions, the modified anti-EGFR antibody exhibits greater activity compared to an unmodified anti-EGFR antibody not containing the amino acid replacement(s). For example, a modified anti-EGFR antibody exhibits at least or about at least 110%, 120%, 130%, 140%, 150%, 160%, 170%, 180%, 190%, 200%, 250%, 300%, 400%, 500%, 600%, 700%, 800%, 900%, 1000% or more of the activity of the unmodified or reference anti-EGFR antibody.

As used herein, the term “the same,” when used in reference to antibody binding affinity, means that the EC₅₀, association constant (Kₐ) or dissociation constant (Kd) is within about 1 to 100 fold or 1 to 10 fold of that of the reference antibody (1-100 fold greater affinity or 1-100 fold less affinity, or any numerical value or range or value within such ranges, than the reference antibody).

As used herein, “substantially the same” when used in reference to EC₅₀, association constant (Kₐ) or dissociation constant (K_{d}), means that the Kₐ, K_{d} or EC₅₀ is within about 5 to 5000 fold greater or less than the Kₐ, K_{d} or EC₅₀, of the reference antibody (5-5000 fold greater or 5-5000 fold less than the reference antibody).

As used herein, “specifically binds” or “immunospecifically binds” with respect to an antibody or antigen-binding fragment thereof are used interchangeably herein and refer to the ability of the antibody or antigen-binding fragment to form one or more noncovalent bonds with a cognate antigen, by noncovalent interactions between the antibody combining site(s) of the antibody and the antigen. Typically, an antibody that immunospecifically binds (or that specifically binds) to EGFR is one that binds to EGFR with an affinity constant Kₐ of about or 1×10⁷ M⁻¹ or 1×10⁸M⁻¹ or greater (or a dissociation constant (K_{d}) of 1×10⁻⁷ M or 1×10⁻⁸M or less). Antibodies or antigen-binding fragments that immunospecifically bind to a particular antigen (e.g., EGFR) can be identified, for example, by immunoassays, such as radioimmunoassays (RIA), enzyme-linked immunosorbent assays (ELISAs), surface plasmon resonance, or other techniques known to those of skill in the art.

As used herein, the term “surface plasmon resonance” refers to an optical phenomenon that allows for the analysis of real-time interactions by detection of alterations in protein concentrations within a biosensor matrix, for example, using the BIAcore system (GE Healthcare Life Sciences).

As used herein, “antibody” refers to immunoglobulins and immunoglobulin fragments, whether natural or partially or wholly synthetically, such as recombinantly, produced, including any fragment thereof containing at least a portion of the variable heavy chain and light region of the immunoglobulin molecule that is sufficient to form an antigen binding site and, when assembled, to specifically bind antigen. Hence, an antibody includes any protein having a binding domain that is homologous or substantially homologous to an immunoglobulin antigen-binding domain (antibody combining site). For example, an antibody refers to an antibody that contains two heavy chains (which can be denoted H and H′) and two light chains (which can be denoted L and L′), where each heavy chain can be a full-length immunoglobulin heavy chain or a portion thereof sufficient to form an antigen binding site (e.g., heavy chains include, but are not limited to, VH chains, VH-CH1 chains and VH-CH1-CH2-CH3 chains), and each light chain can be a full-length light chain or a portion thereof sufficient to form an antigen binding site (e.g., light chains include, but are not limited to, VL chains and VL-CL chains). Each heavy chain (H and H′) pairs with one light chain (L and L′, respectively). Typically, antibodies minimally include all or at least a portion of the variable heavy (VH) chain and/or the variable light (VL) chain. The antibody also can include all or a portion of the constant region.

For purposes herein, the term antibody includes full-length antibodies and portions thereof including antibody fragments, such as anti-EGFR antibody fragments. Antibody fragments, include, but are not limited to, Fab fragments, Fab′ fragments, F(ab)₂ fragments, Fv fragments, disulfide-linked Fvs (dsFv), Fd fragments, Fd′ fragments, single-chain Fvs (scFv), single-chain Fabs (scFab), diabodies, anti-idiotypic (anti-Id) antibodies, or antigen-binding fragments of any of the above. Antibody also includes synthetic antibodies, recombinantly produced antibodies, multispecific antibodies (e.g., bispecific antibodies), human antibodies, non-human antibodies, humanized antibodies, chimeric antibodies, and intrabodies. Antibodies provided herein include members of any immunoglobulin class (e.g., IgG, IgM, IgD, IgE, IgA and IgY), any subclass (e.g., IgG1, IgG2, IgG3, IgG4, IgA1 and IgA2) or sub-subclass (e.g., IgG2a and IgG2b).

As used herein, a form of an antibody refers to a particular structure of an antibody. Antibodies herein include full length antibodies and portions thereof, such as, for example, a Fab fragment or other antibody fragment. Thus, a Fab is a particular form of an antibody.

As used herein, reference to a “corresponding form” of an antibody means that when comparing a property or activity of two antibodies, the property is compared using the same form of the antibody. For example, if it is stated that an antibody has less activity compared to the activity of the corresponding form of a first antibody, that means that a particular form, such as a Fab of that antibody, has less activity compared to the Fab form of the first antibody.

As used herein, a full-length antibody is an antibody having two full-length heavy chains (e.g., VH-CH1-CH2-CH3 or VH-CH1-CH2-CH3-CH4) and two full-length light chains (VL-CL) and hinge regions, such as human antibodies produced by antibody secreting B cells and antibodies with the same domains that are produced synthetically.

As used herein, antibody fragment or antibody portion refers to any portion of a full-length antibody that is less than full length but contains at least a portion of the variable region of the antibody sufficient to form an antigen binding site (e.g., one or more CDRs) and thus retains the binding specificity and/or an activity of the full-length antibody; antibody fragments include antibody derivatives produced by enzymatic treatment of full-length antibodies, as well as synthetically, e.g., recombinantly produced derivatives. Examples of antibody fragments include, but are not limited to, Fab, Fab′, F(ab)₂, single-chain Fvs (scFv), Fv, dsFv, diabody, Fd and Fd fragments (see, for example, Methods in Molecular Biology, Vol 207: Recombinant Antibodies for Cancer Therapy Methods and Protocols (2003); Chapter 1; p 3-25, Kipriyanov). The fragment can include multiple chains linked together, such as by disulfide bridges and/or by peptide linkers. An antibody fragment generally contains at least about 50 amino acids and typically at least 200 amino acids.

As used herein, an Fv antibody fragment is composed of one variable heavy domain (V_{H}) and one variable light (V_{L}) domain linked by noncovalent interactions.

As used herein, a dsFv refers to an Fv with an engineered intermolecular disulfide bond, which stabilizes the V_{H}-V_{L} pair.

As used herein, an Fd fragment is a fragment of an antibody containing a variable domain (V_{H}) and one constant region domain (C_{H}1) of an antibody heavy chain.

As used herein, a Fab fragment is an antibody fragment that results from digestion of a full-length immunoglobulin with papain, or a fragment having the same structure that is produced synthetically, e.g., by recombinant methods. A Fab fragment contains a light chain (containing a V_{L} and C_{L}) and another chain containing a variable domain of a heavy chain (V_{H}) and one constant region domain of the heavy chain (C_{H}1).

As used herein, a F(ab′)₂ fragment is an antibody fragment that results from digestion of an immunoglobulin with pepsin at pH 4.0-4.5, or a fragment having the same structure that is produced synthetically, e.g., by recombinant methods. The F(ab′)₂ fragment essentially contains two Fab fragments where each heavy chain portion contains an additional few amino acids, including cysteine residues that form disulfide linkages joining the two fragments.

As used herein, a Fab′ fragment is a fragment containing one half (one heavy chain and one light chain) of the F(ab′)₂ fragment.

As used herein, an Fd′ fragment is a fragment of an antibody containing one heavy chain portion of a F(ab′)₂ fragment.

As used herein, an Fv′ fragment is a fragment containing only the V_{H} and V_{L} domains of an antibody molecule.

As used herein, hsFv refers to antibody fragments in which the constant domains normally present in a Fab fragment have been substituted with a heterodimeric coiled-coil domain (see, e.g., Arndt et al. (2001) J Mol Biol. 7:312:221-228).

As used herein, an scFv fragment refers to an antibody fragment that contains a variable light chain (V_{L}) and variable heavy chain (V_{H}), covalently connected by a polypeptide linker in any order. The linker is of a length such that the two variable domains are bridged without substantial interference. Exemplary linkers are (Gly-Ser)ₙ residues with some Glu or Lys residues dispersed throughout to increase solubility.

As used herein, diabodies are dimeric scFv; diabodies typically have shorter peptide linkers than scFvs, and preferentially dimerize.

As used herein, a polypeptide “domain” is a part of a polypeptide (a sequence of three or more, generally 5, 10 or more amino acids) that is structurally and/or functionally distinguishable or definable. An exemplary polypeptide domain is a part of the polypeptide that can form an independently folded structure within a polypeptide made up of one or more structural motifs (e.g., combinations of alpha helices and/or beta strands connected by loop regions) and/or that is recognized by a particular functional activity, such as enzymatic activity, dimerization or antigen-binding. A polypeptide can have one or more, typically more than one, distinct domains. For example, the polypeptide can have one or more structural domains and one or more functional domains. A single polypeptide domain can be distinguished based on structure and function. A domain can encompass a contiguous linear sequence of amino acids. Alternatively, a domain can encompass a plurality of non-contiguous amino acid portions, which are non-contiguous along the linear sequence of amino acids of the polypeptide. Typically, a polypeptide contains a plurality of domains. For example, each heavy chain and each light chain of an antibody molecule contains a plurality of immunoglobulin (Ig) domains, each about 110 amino acids in length. Those of skill in the art are familiar with polypeptide domains and can identify them by virtue of structural and/or functional homology with other such domains. For exemplification herein, definitions are provided, but it is understood that it is well within the skill in the art to recognize particular domains by name. If needed, appropriate software can be employed to identify domains.

As used herein, a functional region of a polypeptide is a region of the polypeptide that contains at least one functional domain (which imparts a particular function, such as an ability to interact with a biomolecule, for example, through antigen-binding, DNA binding, ligand binding, or dimerization, or by enzymatic activity, for example, kinase activity or proteolytic activity); exemplary functional regions of polypeptides are antibody domains, such as V_{H}, V_{L}, C_{H}, C_{L}, and portions thereof, such as CDRs, including CDR1, CDR2 and CDR3, or antigen-binding portions, such as antibody combining sites.

As used herein, a structural region of a polypeptide is a region of the polypeptide that contains at least one structural domain.

As used herein, an Ig domain is a domain, recognized as such by those in the art, that is distinguished by a structure, called the Immunoglobulin (Ig) fold, which contains two beta-pleated sheets, each containing anti-parallel beta strands of amino acids connected by loops. The two beta sheets in the Ig fold are sandwiched together by hydrophobic interactions and a conserved intra-chain disulfide bond. Individual immunoglobulin domains within an antibody chain further can be distinguished based on function. For example, a light chain contains one variable region domain (VL) and one constant region domain (CL), while a heavy chain contains one variable region domain (VH) and three or four constant region domains (CH). Each VL, CL, VH, and CH domain is an example of an immunoglobulin domain.

As used herein, a variable domain with reference to an antibody is a specific Ig domain of an antibody heavy or light chain that contains a sequence of amino acids that varies among different antibodies. Each light chain and each heavy chain has one variable region domain (VL and VH). The variable domains provide antigen specificity, and thus are responsible for antigen recognition. Each variable region contains CDRs that are part of the antigen binding site domain and framework regions (FRs).

As used herein, “hypervariable region,” “HV,” “complementarity-determining region,” “CDR” and “antibody CDR” are used interchangeably to refer to one of a plurality of portions within each variable region that together form an antigen binding site of an antibody. Each variable region domain contains three CDRs, named CDR1, CDR2, and CDR3. The three CDRs are non-contiguous along the linear amino acid sequence, but are proximate in the folded polypeptide. The CDRs are located within the loops that join the parallel strands of the beta sheets of the variable domain.

As used herein, “antigen-binding domain,” “antigen-binding site,” “antigen combining site” and “antibody combining site” are used synonymously to refer to a domain within an antibody that recognizes and physically interacts with the cognate antigen. A native conventional full-length antibody molecule has two conventional antigen-binding sites, each containing portions of a heavy chain variable region and portions of a light chain variable region. A conventional antigen-binding site contains the loops that connect the anti-parallel beta strands within the variable region domains. The antigen combining sites can contain other portions of the variable region domains. Each conventional antigen-binding site contains three hypervariable regions from the heavy chain and three hypervariable regions from the light chain. The hypervariable regions also are called complementarity-determining regions (CDRs).

As used herein, “portion thereof” with reference to an antibody heavy or light chain or variable heavy or light chain refers to a contiguous portion thereof that is sufficient to form an antigen binding site such that, when assembled into an antibody containing a heavy and light chain, it contains at least 1 or 2, typically 3, 4, 5 or all 6 CDRs of the variable heavy (VH) and variable light (VL) chains sufficient to retain at least a portion of the binding specificity of the corresponding full-length antibody containing all 6 CDRs. Generally, a sufficient antigen binding site requires CDR3 of the heavy chain (CDRH3). It typically further requires the CDR3 of the light chain (CDRL3). As described herein, one of skill in the art knows and can identify the CDRs based on Kabat or Chothia numbering (see e.g., Kabat, E. A. et al. (1991) Sequences of Proteins of Immunological Interest, Fifth Edition, U.S. Department of Health and Human Services, NIH Publication No. 91-3242, and Chothia, C. et al. (1987)J. Mol. Biol. 196:901-917).

As used herein, framework regions (FRs) are the domains within the antibody variable region domains that are located within the beta sheets; the FR regions are comparatively more conserved, in terms of their amino acid sequences, than the hypervariable regions.

As used herein, a constant region domain is a domain in an antibody heavy or light chain that contains a sequence of amino acids that is comparatively more conserved among antibodies than the variable region domain. Each light chain has a single light chain constant region (CL) domain and each heavy chain contains one or more heavy chain constant region (CH) domains, which include, CH1, CH2, CH3 and CH4. Full-length IgA, IgD and IgG isotypes contain CH1, CH2, CH3 and a hinge region, while IgE and IgM contain CH1, CH2, CH3 and CH4. CH1 and CL domains extend the Fab arm of the antibody molecule, thus contributing to the interaction with antigen and rotation of the antibody arms. Antibody constant regions can serve effector functions, such as, but not limited to, clearance of antigens, pathogens and toxins to which the antibody specifically binds, e.g., through interactions with various cells, biomolecules and tissues.

As used herein, “Kabat numbering” refers to the index numbering of the IgG1 Kabat antibody (see e.g., Kabat, E. A. et al. (1991) Sequences of Proteins of Immunological Interest, Fifth Edition, U.S. Department of Health and Human Services, NIH Publication No. 91-3242). For example, based on Kabat numbering, CDR-LI corresponds to residues L24-L34; CDR-L2 corresponds to residues L50-L56; CDR-L3 corresponds to residues L89-L97; CDR-H1 corresponds to residues H31-H35, 35a or 35b depending on the length; CDR-H2 corresponds to residues H50-H65; and CDR-H3 corresponds to residues H95-H102. One of skill in the art can identify regions of the constant region using Kabat. Tables 1 and 2 set forth corresponding residues using kabat numbering and EU numbering schemes for the exemplary antibody cetuximab.

As used herein, “EU numbering” or “EU index” refer to the numbering scheme of the EU antibody described in Edelman et al., Proc Natl. Acad. Sci. USA 63 (1969) 78-85. “EU index as in Kabat” refers to EU index numbering of the human IgG1 Kabat antibody as set forth in Kabat, E. A. et al. (1991) Sequences of Proteins of Immunological Interest, Fifth Edition, U.S. Department of Health and Human Services, NIH Publication No. 91-3242. EU numbering or EU numbering as in Kabat are frequently used by those of skill in the art to number amino acid residues of the Fc regions of the light and heavy antibody chains. For example, one of skill in the art can identify regions of the constant region using EU numbering. For example, the CL domain corresponds to residues L108-L216 according to Kabat numbering or L108-L214 according to EU numbering. CH1 corresponds to residues 118-215 (EU numbering) or 114-223 (Kabat numbering); CH2 corresponds to residues 231-340 (EU numbering) or 244-360 (Kabat numbering); CH3 corresponds to residues 341-446 (EU numbering) or 361-478 (Kabat numbering) domain corresponds to; CDR-L2 corresponds to residues L50-L56; CDR-L3 corresponds to residues L89-L97; CDR-H1 corresponds to residues H31-H35, 35a or 35b depending on the length; CDR-H2 corresponds to residues H50-H65; and CDR-H3 corresponds to residues H95-H102. Tables 1 and 2 set forth corresponding residues using Kabat and EU numbering for the exemplary antibody cetuximab. The top row (bold) sets forth the amino acid residue number; the second row (bold) provides the 1-letter code for the amino acid residue at the position indicated by the number in the top row; the third row (italic) indicates the corresponding Kabat number according to Kabat numbering; and the fourth row (not-bold, not-italic) indicates the corresponding EU index number according to EU numbering.

TABLE 1
Kabat and EU Numbering of Cetuximab Light Chain

| 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|
| D | I | L | L | T | Q | S | P | V | I | L | S | V | S | P |
| 1 | 2 | 3 | 4 | 5 | 6 | 7 | 8 | 9 | 10 | 11 | 12 | 13 | 14 | 15 |
| 1 | 2 | 3 | 4 | 5 | 6 | 7 | 8 | 9 | 10 | 11 | 12 | 13 | 14 | 15 |
| 16 | 17 | 18 | 19 | 20 | 21 | 22 | 23 | 24 | 25 | 26 | 27 | 28 | 29 | 30 |
| G | E | R | V | S | F | S | C | R | A | S | Q | S | I | G |
| 16 | 17 | 18 | 19 | 20 | 21 | 22 | 23 | 24 | 25 | 26 | 27 | 28 | 29 | 30 |
| 16 | 17 | 18 | 19 | 20 | 21 | 22 | 23 | 24 | 25 | 26 | 27 | 28 | 29 | 30 |
| 31 | 32 | 33 | 34 | 35 | 36 | 37 | 38 | 39 | 40 | 41 | 42 | 43 | 44 | 45 |
| T | N | I | H | W | Y | Q | Q | R | T | N | G | S | P | R |
| 31 | 32 | 33 | 34 | 35 | 36 | 37 | 38 | 39 | 40 | 41 | 42 | 43 | 44 | 45 |
| 31 | 32 | 33 | 34 | 35 | 36 | 37 | 38 | 39 | 40 | 41 | 42 | 43 | 44 | 45 |
| 46 | 47 | 48 | 49 | 50 | 51 | 52 | 53 | 54 | 55 | 56 | 57 | 58 | 59 | 60 |
| L | L | I | K | Y | A | S | E | S | I | S | G | I | P | S |
| 46 | 47 | 48 | 49 | 50 | 51 | 52 | 53 | 54 | 55 | 56 | 57 | 58 | 59 | 60 |
| 46 | 47 | 48 | 49 | 50 | 51 | 52 | 53 | 54 | 55 | 56 | 57 | 58 | 59 | 60 |
| 61 | 62 | 63 | 64 | 65 | 66 | 67 | 68 | 69 | 70 | 71 | 72 | 73 | 74 | 75 |
| R | F | S | G | S | G | S | G | T | D | F | T | L | S | I |
| 61 | 62 | 63 | 64 | 65 | 66 | 67 | 68 | 69 | 70 | 77 | 72 | 73 | 74 | 75 |
| 61 | 62 | 63 | 64 | 65 | 66 | 67 | 68 | 69 | 70 | 71 | 72 | 73 | 74 | 75 |
| 76 | 77 | 78 | 79 | 80 | 81 | 82 | 83 | 84 | 85 | 86 | 87 | 88 | 89 | 90 |
| N | S | V | E | S | E | D | I | A | D | Y | Y | C | Q | Q |
| 76 | 77 | 78 | 79 | 80 | 81 | 82 | 83 | 84 | 85 | 86 | 87 | 88 | 89 | 90 |
| 76 | 77 | 78 | 79 | 80 | 81 | 82 | 83 | 84 | 85 | 86 | 87 | 88 | 89 | 90 |
| 91 | 92 | 93 | 94 | 95 | 96 | 97 | 98 | 99 | 100 | 101 | 102 | 103 | 104 | 105 |
| N | N | N | W | P | T | T | F | G | A | G | T | K | L | E |
| 91 | 92 | 93 | 94 | 95 | 96 | 97 | 98 | 99 | 100 | 101 | 102 | 103 | 104 | 105 |
| 91 | 92 | 93 | 94 | 95 | 96 | 97 | 98 | 99 | 100 | 101 | 102 | 103 | 104 | 105 |
| 106 | 107 | 108 | 109 | 110 | 111 | 112 | 113 | 114 | 115 | 116 | 117 | 118 | 119 | 120 |
| L | K | R | T | V | A | A | P | S | V | F | I | F | P | P |
| 106 | 107 | 108 | 109 | 110 | 111 | 112 | 113 | 114 | 115 | 116 | 117 | 118 | 119 | 120 |
| 106 | 107 | 108 | 109 | 110 | 111 | 112 | 113 | 114 | 115 | 116 | 117 | 118 | 119 | 120 |
| 121 | 122 | 123 | 124 | 125 | 126 | 127 | 128 | 129 | 130 | 131 | 132 | 133 | 134 | 135 |
| S | D | E | Q | L | K | S | G | T | A | S | V | V | C | L |
| 121 | 122 | 123 | 124 | 125 | 126 | 127 | 128 | 129 | 130 | 137 | 132 | 133 | 134 | 135 |
| 121 | 122 | 123 | 124 | 125 | 126 | 127 | 128 | 129 | 130 | 131 | 132 | 133 | 134 | 135 |
| 136 | 137 | 138 | 139 | 140 | 141 | 142 | 143 | 144 | 145 | 146 | 147 | 148 | 149 | 150 |
| L | N | N | F | Y | P | R | E | A | K | V | Q | W | K | V |
| 136 | 137 | 138 | 139 | 140 | 147 | 142 | 143 | 144 | 145 | 146 | 147 | 148 | 149 | 150 |
| 136 | 137 | 138 | 139 | 140 | 141 | 142 | 143 | 144 | 145 | 146 | 147 | 148 | 149 | 150 |
| 151 | 152 | 153 | 154 | 155 | 156 | 157 | 158 | 159 | 160 | 161 | 162 | 163 | 164 | 165 |
| D | N | A | L | Q | S | G | N | S | Q | E | S | V | T | E |
| 151 | 152 | 153 | 154 | 155 | 156 | 157 | 158 | 159 | 160 | 161 | 162 | 163 | 164 | 165 |
| 151 | 152 | 153 | 154 | 155 | 156 | 157 | 158 | 159 | 160 | 161 | 162 | 163 | 164 | 165 |
| 166 | 167 | 168 | 169 | 170 | 171 | 172 | 173 | 174 | 175 | 176 | 177 | 178 | 179 | 180 |
| Q | D | S | K | D | S | T | Y | S | L | S | S | T | L | T |
| 166 | 167 | 168 | 169 | 170 | 171 | 172 | 173 | 174 | 175 | 176 | 177 | 178 | 179 | 180 |
| 166 | 167 | 168 | 169 | 170 | 171 | 172 | 173 | 174 | 175 | 176 | 177 | 178 | 179 | 180 |
| 181 | 182 | 183 | 184 | 185 | 186 | 187 | 188 | 189 | 190 | 191 | 192 | 193 | 194 | 195 |
| L | S | K | A | D | Y | E | K | H | K | V | Y | A | C | E |
| 181 | 182 | 183 | 184 | 185 | 186 | 187 | 188 | 189 | 190 | 191 | 192 | 193 | 194 | 195 |
| 181 | 182 | 183 | 184 | 185 | 186 | 187 | 188 | 189 | 190 | 191 | 192 | 193 | 194 | 195 |
| 196 | 197 | 198 | 199 | 200 | 201 | 202 | 203 | 204 | 205 | 206 | 207 | 208 | 209 | 210 |
| V | T | H | Q | G | L | S | S | P | V | T | K | S | F | N |
| 196 | 197 | 198 | 199 | 200 | 201 | 202 | 203 | 204 | 205 | 206 | 207 | 208 | 209 | 210 |
| 196 | 197 | 198 | 199 | 200 | 201 | 202 | 203 | 204 | 205 | 206 | 207 | 208 | 209 | 210 |

| | | |
|---|---|---|
| 211 | 212 | 213 |
| R | G | A |
| 211 | 212 | 213 |
| 211 | 212 | 213 |

TABLE 2
Kabat and EU Numbering of Cetuximab Heavy Chain

| 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|
| Q | V | Q | L | K | Q | S | G | P | G | L | V | Q | P | S |
| 1 | 2 | 3 | 4 | 5 | 6 | 7 | 8 | 9 | 10 | 11 | 12 | 13 | 14 | 15 |
| 1 | 2 | 3 | 4 | 5 | 6 | 7 | 8 | 9 | 10 | 11 | 12 | 13 | 14 | 15 |
| 16 | 17 | 18 | 19 | 20 | 21 | 22 | 23 | 24 | 25 | 26 | 27 | 28 | 29 | 30 |
| Q | S | L | S | I | T | C | T | V | S | G | F | S | L | T |
| 16 | 17 | 18 | 19 | 20 | 21 | 22 | 23 | 24 | 25 | 26 | 27 | 28 | 29 | 30 |
| 16 | 17 | 18 | 19 | 20 | 21 | 22 | 23 | 24 | 25 | 26 | 27 | 28 | 29 | 30 |
| 31 | 32 | 33 | 34 | 35 | 36 | 37 | 38 | 39 | 40 | 41 | 42 | 43 | 44 | 45 |
| N | Y | G | V | H | W | V | R | Q | S | P | G | K | G | L |
| 31 | 32 | 33 | 34 | 35 | 36 | 37 | 38 | 39 | 40 | 41 | 42 | 43 | 44 | 45 |
| 31 | 32 | 33 | 34 | 35 | 36 | 37 | 38 | 39 | 40 | 41 | 42 | 43 | 44 | 45 |
| 46 | 47 | 48 | 49 | 50 | 51 | 52 | 53 | 54 | 55 | 56 | 57 | 58 | 59 | 60 |
| E | W | L | G | V | I | W | S | G | G | N | T | D | Y | N |
| 46 | 47 | 48 | 49 | 50 | 51 | 52 | 53 | 54 | 55 | 56 | 57 | 58 | 59 | 60 |
| 46 | 47 | 48 | 49 | 51 | 52 | 53 | 54 | 55 | 56 | 57 | 58 | 59 | 60 | 61 |
| 61 | 62 | 63 | 64 | 65 | 66 | 67 | 68 | 69 | 70 | 71 | 72 | 73 | 74 | 75 |
| T | P | F | T | S | R | L | S | I | N | K | D | N | S | K |
| 61 | 62 | 63 | 64 | 65 | 66 | 67 | 68 | 69 | 70 | 71 | 72 | 73 | 74 | 75 |
| 62 | 63 | 64 | 65 | 66 | 67 | 68 | 69 | 70 | 71 | 72 | 73 | 74 | 75 | 76 |
| 76 | 77 | 78 | 79 | 80 | 81 | 82 | 83 | 84 | 85 | 86 | 87 | 88 | 89 | 90 |
| S | Q | V | F | F | K | M | N | S | L | Q | S | N | D | T |
| 76 | 77 | 78 | 79 | 80 | 81 | 82 | 82A | 82B | 82C | 83 | 84 | 85 | 86 | 87 |
| 77 | 78 | 79 | 80 | 81 | 82 | 83 | 84 | 85 | 86 | 87 | 88 | 89 | 90 | 91 |
| 91 | 92 | 93 | 94 | 95 | 96 | 97 | 98 | 99 | 100 | 101 | 102 | 103 | 104 | 105 |
| A | I | Y | Y | C | A | R | A | L | T | Y | Y | D | Y | E |
| 88 | 89 | 90 | 91 | 92 | 93 | 94 | 95 | 96 | 97 | 98 | 99 | 100 | 100A | 100B |
| 92 | 93 | 94 | 95 | 96 | 97 | 98 | 99 | 100 | 101 | 102 | 103 | 104 | 105 | 106 |
| 106 | 107 | 108 | 109 | 110 | 111 | 112 | 113 | 114 | 115 | 116 | 117 | 118 | 119 | 120 |
| F | A | Y | W | G | Q | G | T | L | V | T | V | S | A | A |
| 100C | 101 | 102 | 103 | 104 | 105 | 106 | 107 | 108 | 109 | 110 | 111 | 112 | 113 | 114 |
| — | 107 | 108 | 109 | 110 | — | 111 | — | 112 | 113 | 114 | 115 | 116 | 117 | 118 |
| 121 | 122 | 123 | 124 | 125 | 126 | 127 | 128 | 129 | 130 | 131 | 132 | 133 | 134 | 135 |
| S | T | K | G | P | S | V | F | P | L | A | P | S | S | K |
| 115 | 116 | 117 | 118 | 119 | 120 | 121 | 122 | 123 | 124 | 125 | 126 | 127 | 128 | 129 |
| 119 | 120 | 121 | 122 | 123 | 124 | 125 | 126 | 127 | 128 | 129 | 130 | 131 | 132 | 133 |
| 136 | 137 | 138 | 139 | 140 | 141 | 142 | 143 | 144 | 145 | 146 | 147 | 148 | 149 | 150 |
| S | T | S | G | G | T | A | A | L | G | C | L | V | K | D |
| 130 | 133 | 134 | 135 | 136 | 137 | 138 | 139 | 140 | 141 | 142 | 143 | 144 | 145 | 146 |
| 134 | 135 | 136 | 137 | 138 | 139 | 140 | 141 | 142 | 143 | 144 | 145 | 146 | 147 | 148 |
| 151 | 152 | 153 | 154 | 155 | 156 | 157 | 158 | 159 | 160 | 161 | 162 | 163 | 164 | 165 |
| Y | F | P | E | P | V | T | V | S | W | N | S | G | A | L |
| 147 | 148 | 149 | 150 | 151 | 152 | 153 | 154 | 156 | 157 | 162 | 163 | 164 | 165 | 166 |
| 149 | 150 | 151 | 152 | 153 | 154 | 155 | 156 | 157 | 158 | 159 | 160 | 161 | 162 | 163 |
| 166 | 167 | 168 | 169 | 170 | 171 | 172 | 173 | 174 | 175 | 176 | 177 | 178 | 179 | 180 |
| T | S | G | V | H | T | F | P | A | V | L | Q | S | S | G |
| 167 | 168 | 169 | 171 | 172 | 173 | 174 | 175 | 176 | 177 | 178 | 179 | 180 | 182 | 183 |
| 164 | 165 | 166 | 167 | 168 | 169 | 170 | 171 | 172 | 173 | 174 | 175 | 176 | 177 | 178 |
| 181 | 182 | 183 | 184 | 185 | 186 | 187 | 188 | 189 | 190 | 191 | 192 | 193 | 194 | 195 |
| L | Y | S | L | S | S | V | V | T | V | P | S | S | S | L |
| 184 | 185 | 186 | 187 | 188 | 189 | 190 | 191 | 192 | 193 | 194 | 195 | 196 | 197 | 198 |
| 179 | 180 | 181 | 182 | 183 | 184 | 185 | 186 | 187 | 188 | 189 | 190 | 191 | 192 | 193 |
| 196 | 197 | 198 | 199 | 200 | 201 | 202 | 203 | 204 | 205 | 206 | 207 | 208 | 209 | 210 |
| G | T | Q | T | Y | I | C | N | V | N | H | K | P | S | N |
| 199 | 200 | 203 | 205 | 206 | 207 | 208 | 209 | 210 | 211 | 212 | 213 | 214 | 215 | 216 |
| 194 | 195 | 196 | 197 | 198 | 199 | 200 | 201 | 202 | 203 | 204 | 205 | 206 | 207 | 208 |
| 211 | 212 | 213 | 214 | 215 | 216 | 217 | 218 | 219 | 220 | 221 | 222 | 223 | 224 | 225 |
| T | K | V | D | K | R | V | E | P | K | S | C | D | K | T |
| 217 | 218 | 219 | 220 | 221 | 222 | 223 | 226 | 227 | 228 | 232 | 233 | 234 | 235 | 236 |
| 209 | 210 | 211 | 212 | 213 | 214 | 215 | 216 | 217 | 218 | 219 | 220 | 221 | 222 | 223 |
| 226 | 227 | 228 | 229 | 230 | 231 | 232 | 233 | 234 | 235 | 236 | 237 | 238 | 239 | 240 |
| H | T | C | P | P | C | P | A | P | E | L | L | G | G | P |
| 237 | 238 | 239 | 240 | 241 | 242 | 243 | 244 | 245 | 246 | 247 | 248 | 249 | 250 | 251 |
| 224 | 225 | 226 | 227 | 228 | 229 | 230 | 231 | 232 | 233 | 234 | 235 | 236 | 237 | 238 |
| 241 | 242 | 243 | 244 | 245 | 246 | 247 | 248 | 249 | 250 | 251 | 252 | 253 | 254 | 255 |
| S | V | F | L | F | P | P | K | P | K | D | T | L | M | I |
| 252 | 253 | 254 | 255 | 256 | 257 | 258 | 259 | 260 | 261 | 262 | 263 | 264 | 265 | 266 |
| 239 | 240 | 241 | 242 | 243 | 244 | 245 | 246 | 247 | 248 | 249 | 250 | 251 | 252 | 253 |
| 256 | 257 | 258 | 259 | 260 | 261 | 262 | 263 | 264 | 265 | 266 | 267 | 268 | 269 | 270 |
| S | R | T | P | E | V | T | C | V | V | V | D | V | S | H |
| 267 | 268 | 269 | 270 | 271 | 272 | 273 | 274 | 275 | 276 | 277 | 278 | 279 | 280 | 281 |
| 254 | 255 | 256 | 257 | 258 | 259 | 260 | 261 | 262 | 263 | 264 | 265 | 266 | 267 | 268 |
| 271 | 272 | 273 | 274 | 275 | 276 | 277 | 278 | 279 | 280 | 281 | 282 | 283 | 284 | 285 |
| E | D | P | E | V | K | F | N | W | Y | V | D | G | V | E |
| 282 | 283 | 284 | 285 | 286 | 287 | 288 | 289 | 290 | 291 | 292 | 295 | 296 | 299 | 300 |
| 269 | 270 | 271 | 272 | 273 | 274 | 275 | 276 | 277 | 278 | 279 | 280 | 281 | 282 | 283 |
| 286 | 287 | 288 | 289 | 290 | 291 | 292 | 293 | 294 | 295 | 296 | 297 | 298 | 299 | 300 |
| V | H | N | A | K | T | K | P | R | E | E | Q | Y | N | S |
| 301 | 302 | 303 | 304 | 305 | 306 | 307 | 308 | 309 | 310 | 311 | 312 | 313 | 314 | 317 |
| 284 | 285 | 286 | 287 | 288 | 289 | 290 | 291 | 292 | 293 | 294 | 295 | 296 | 297 | 298 |
| 301 | 302 | 303 | 304 | 305 | 306 | 307 | 308 | 309 | 310 | 311 | 312 | 313 | 314 | 315 |
| T | Y | R | V | V | S | V | L | T | V | L | H | Q | D | W |
| 318 | 319 | 320 | 321 | 322 | 323 | 324 | 325 | 326 | 327 | 328 | 329 | 330 | 331 | 332 |
| 299 | 300 | 301 | 302 | 303 | 304 | 305 | 306 | 307 | 308 | 309 | 310 | 311 | 312 | 313 |
| 316 | 317 | 318 | 319 | 320 | 321 | 322 | 323 | 324 | 325 | 326 | 327 | 328 | 329 | 330 |
| L | N | G | K | E | Y | K | C | K | V | S | N | K | A | L |
| 333 | 334 | 335 | 336 | 337 | 338 | 339 | 340 | 341 | 342 | 343 | 344 | 345 | 346 | 347 |
| 314 | 315 | 316 | 317 | 318 | 319 | 320 | 321 | 322 | 323 | 324 | 325 | 326 | 327 | 328 |
| 331 | 332 | 333 | 334 | 335 | 336 | 337 | 338 | 339 | 340 | 341 | 342 | 343 | 344 | 345 |
| P | A | P | I | E | K | T | I | S | K | A | K | G | Q | P |
| 348 | 349 | 350 | 351 | 352 | 353 | 354 | 355 | 357 | 358 | 359 | 360 | 361 | 363 | 364 |
| 329 | 330 | 331 | 332 | 333 | 334 | 335 | 336 | 337 | 338 | 339 | 340 | 341 | 342 | 343 |
| 346 | 347 | 348 | 349 | 350 | 351 | 352 | 353 | 354 | 355 | 356 | 357 | 358 | 359 | 360 |
| R | E | P | Q | V | Y | T | L | P | P | S | R | D | E | L |
| 365 | 366 | 367 | 368 | 369 | 370 | 371 | 372 | 373 | 374 | 375 | 376 | 377 | 378 | 381 |
| 344 | 345 | 346 | 347 | 348 | 349 | 350 | 351 | 352 | 353 | 354 | 355 | 356 | 357 | 358 |
| 361 | 362 | 363 | 364 | 365 | 366 | 367 | 368 | 369 | 370 | 371 | 372 | 373 | 374 | 375 |
| T | K | N | Q | V | S | L | T | C | L | V | K | G | F | Y |
| 382 | 383 | 384 | 385 | 386 | 387 | 388 | 389 | 390 | 391 | 392 | 393 | 394 | 395 | 396 |
| 359 | 360 | 361 | 362 | 363 | 364 | 365 | 366 | 367 | 368 | 369 | 370 | 371 | 372 | 373 |
| 376 | 377 | 378 | 379 | 380 | 381 | 382 | 383 | 384 | 385 | 386 | 387 | 388 | 389 | 390 |
| P | S | D | I | A | V | E | W | E | S | N | G | Q | P | E |
| 397 | 398 | 399 | 400 | 401 | 402 | 405 | 406 | 407 | 408 | 410 | 411 | 414 | 415 | 416 |
| 374 | 375 | 376 | 377 | 378 | 379 | 380 | 381 | 382 | 383 | 384 | 385 | 386 | 387 | 388 |
| 391 | 392 | 393 | 394 | 395 | 396 | 397 | 398 | 399 | 400 | 401 | 402 | 403 | 404 | 405 |
| N | N | Y | K | T | T | P | P | V | L | D | S | D | G | S |
| 417 | 418 | 419 | 420 | 421 | 422 | 423 | 424 | 425 | 426 | 427 | 428 | 430 | 433 | 434 |
| 389 | 390 | 391 | 392 | 393 | 394 | 395 | 396 | 397 | 398 | 399 | 400 | 401 | 402 | 403 |
| 406 | 407 | 408 | 409 | 410 | 411 | 412 | 413 | 414 | 415 | 416 | 417 | 418 | 419 | 420 |
| F | F | L | Y | S | K | L | T | V | D | K | S | R | W | Q |
| 435 | 436 | 437 | 438 | 439 | 440 | 441 | 442 | 443 | 444 | 445 | 446 | 447 | 448 | 449 |
| 404 | 405 | 406 | 407 | 408 | 409 | 410 | 411 | 412 | 413 | 414 | 415 | 416 | 417 | 418 |
| 421 | 422 | 423 | 424 | 425 | 426 | 427 | 428 | 429 | 430 | 431 | 432 | 433 | 434 | 435 |
| Q | G | N | V | F | S | C | S | V | M | H | E | A | L | H |
| 450 | 451 | 452 | 453 | 454 | 455 | 456 | 457 | 458 | 459 | 460 | 461 | 462 | 463 | 464 |
| 419 | 420 | 421 | 422 | 423 | 424 | 425 | 426 | 427 | 428 | 429 | 430 | 431 | 432 | 433 |

| | | | | | | | | | | | | | |
|---|---|---|---|---|---|---|---|---|---|---|---|---|---|
| 436 | 437 | 438 | 439 | 440 | 441 | 442 | 443 | 444 | 445 | 446 | 447 | 448 | 449 |
| N | H | Y | T | Q | K | S | L | S | L | S | P | G | K |
| 465 | 466 | 467 | 468 | 469 | 470 | 471 | 472 | 473 | 474 | 475 | 476 | 477 | 478 |
| 434 | 435 | 436 | 437 | 438 | 439 | 440 | 441 | 442 | 443 | 444 | 445 | 446 | — |

As used herein, “antibody hinge region” or “hinge region” refers to a polypeptide region that exists naturally in the heavy chain of the gamma, delta and alpha antibody isotypes, between the C_{H}1 and C_{H}2 domains that has no homology with the other antibody domains. This region is rich in proline residues and gives the IgG, IgD and IgA antibodies flexibility, allowing the two “arms” (each containing one antibody combining site) of the Fab portion to be mobile, assuming various angles with respect to one another as they bind antigen. This flexibility allows the Fab arms to move in order to align the antibody combining sites to interact with epitopes on cell surfaces or other antigens. Two interchain disulfide bonds within the hinge region stabilize the interaction between the two heavy chains. In some embodiments provided herein, the synthetically produced antibody fragments contain one or more hinge regions, for example, to promote stability via interactions between two antibody chains. Hinge regions are examples of dimerization domains.

As used herein, the phrase “derived from” when referring to antibody fragments derived from another antibody, such as a monoclonal antibody, refers to the engineering of antibody fragments (e.g., Fab, F(ab′), F(ab′)₂, single-chain Fv (scFv), Fv, dsFv, diabody, Fd and Fd′ fragments) that retain the binding specificity of the original antibody. Such fragments can be derived by a variety of methods known in the art, including, but not limited to, enzymatic cleavage, chemical crosslinking, recombinant means or combinations thereof. Generally, the derived antibody fragment shares the identical or substantially identical heavy chain variable region (V_{H}) and light chain variable region (V_{L}) of the parent antibody, such that the antibody fragment and the parent antibody bind the same epitope.

As used herein, a “parent antibody” or “source antibody” refers the to an antibody from which an antibody fragment (e.g., Fab, F(ab′), F(ab)₂, single-chain Fv (scFv), Fv, dsFv, diabody, Fd and Fd′ fragments) is derived.

As used herein, the term “epitope” refers to any antigenic determinant on an antigen to which the paratope of an antibody binds. Epitopic determinants typically contain chemically active surface groupings of molecules such as amino acids or sugar side chains and typically have specific three dimensional structural characteristics, as well as specific charge characteristics.

As used herein, humanized antibodies refer to antibodies that are modified to include “human” sequences of amino acids so that administration to a human does not provoke an immune response. A humanized antibody typically contains complementarity determining regions (CDRs or hypervariable loops) derived from a non-human species immunoglobulin and the remainder of the antibody molecule derived mainly from a human immunoglobulin. Methods for preparation of such antibodies are known. For example, DNA encoding a monoclonal antibody can be altered by recombinant DNA techniques to encode an antibody in which the amino acid composition of the non-variable regions is based on human antibodies. Methods for identifying such regions are known, including computer programs, which are designed for identifying the variable and non-variable regions of immunoglobulins. Hence, in general, the humanized antibody will contain substantially all of at least one, and typically two, variable domains, in which all or substantially all of the hypervariable loops (e.g., CDRs) correspond to those of a non-human immunoglobulin and all or substantially all of the FRs are those of a human immunoglobulin sequence. The humanized antibody optionally also will contain at least a portion of an immunoglobulin constant region (Fc), typically that of a human immunoglobulin.

As used herein, a multimerization domain refers to a sequence of amino acids that promotes stable interaction of a polypeptide molecule with one or more additional polypeptide molecules, each containing a complementary multimerization domain, which can be the same or a different multimerization domain to form a stable multimer with the first domain. Generally, a polypeptide is joined directly or indirectly to the multimerization domain. Exemplary multimerization domains include the immunoglobulin sequences or portions thereof, leucine zippers, hydrophobic regions, hydrophilic regions, and compatible protein-protein interaction domains. The multimerization domain, for example, can be an immunoglobulin constant region or domain, such as, for example, the Fc domain or portions thereof from IgG, including IgG1, IgG2, IgG3 or IgG4 subtypes, IgA, IgE, IgD and IgM and modified forms thereof

As used herein, dimerization domains are multimerization domains that facilitate interaction between two polypeptide sequences (such as, but not limited to, antibody chains). Dimerization domains include, but are not limited to, an amino acid sequence containing a cysteine residue that facilitates formation of a disulfide bond between two polypeptide sequences, such as all or part of a full-length antibody hinge region, or one or more dimerization sequences, which are sequences of amino acids known to promote interaction between polypeptides (e.g., leucine zippers, GCN4 zippers).

As used herein, “Fc” or “Fc region” or “Fe domain” refers to a polypeptide containing the constant region of an antibody heavy chain, excluding the first constant region immunoglobulin domain. Thus, Fc refers to the last two constant region immunoglobulin domains of IgA, IgD, and IgE, or the last three constant region immunoglobulin domains of IgE and IgM. Optionally, an Fc domain can include all or part of the flexible hinge N-terminal to these domains. For IgA and IgM, Fc can include the J chain. For an exemplary Fc domain of IgG, Fc contains immunoglobulin domains Cγ2 and Cγ3, and optionally, all or part of the hinge between Cγ1 and Cγ2. The boundaries of the Fc region can vary, but typically, include at least part of the hinge region. In addition, Fc also includes any allelic or species variant or any variant or modified form, such as any variant or modified form that alters the binding to an FcR or alters an Fc-mediated effector function.

As used herein, “Fc chimera” refers to a chimeric polypeptide in which one or more polypeptides is linked, directly or indirectly, to an Fc region or a derivative thereof. Typically, an Fc chimera combines the Fc region of an immunoglobulin with another polypeptide. Derivatives of or modified Fc polypeptides are known to those of skill in the art.

As used herein, a chimeric polypeptide refers to a polypeptide that contains portions from at least two different polypeptides or from two non-contiguous portions of a single polypeptide. Thus, a chimeric polypeptide generally includes a sequence of amino acid residues from all or part of one polypeptide and a sequence of amino acids from all or part of another different polypeptide. The two portions can be linked directly or indirectly and can be linked via peptide bonds, other covalent bonds or other non-covalent interactions of sufficient strength to maintain the integrity of a substantial portion of the chimeric polypeptide under equilibrium conditions and physiologic conditions, such as in isotonic pH 7 buffered saline.

As used herein, a fusion protein is a polypeptide engineered to contain sequences of amino acids corresponding to two distinct polypeptides, which are joined together, such as by expressing the fusion protein from a vector containing two nucleic acids, encoding the two polypeptides, in close proximity, e.g., adjacent, to one another along the length of the vector. Accordingly, a fusion protein refers to a chimeric protein containing two, or portions from two, or more proteins or peptides that are linked directly or indirectly via peptide bonds. The two molecules can be adjacent in the construct or separated by a linker, or spacer polypeptide.

As used herein, “linker” or “spacer” peptide refers to short sequences of amino acids that join two polypeptide sequences (or nucleic acid encoding such an amino acid sequence). “Peptide linker” refers to the short sequence of amino acids joining the two polypeptide sequences. Exemplary of polypeptide linkers are linkers joining a peptide transduction domain to an antibody or linkers joining two antibody chains in a synthetic antibody fragment such as an scFv fragment. Linkers are well-known and any known linkers can be used in the provided methods. Exemplary polypeptide linkers include (Gly-Ser)ₙ amino acid sequences, with some Glu or Lys residues dispersed throughout to increase solubility. Other exemplary linkers are described herein; any of these and other known linkers can be used with the provided compositions and methods.

As used herein, a “tag” or an “epitope tag” refers to a sequence of amino acids, typically added to the N- or C-terminus of a polypeptide, such as an antibody provided herein. The inclusion of tags fused to a polypeptide can facilitate polypeptide purification and/or detection. Typically, a tag or tag polypeptide refers to a polypeptide that has enough residues to provide an epitope recognized by an antibody or can serve for detection or purification, yet is short enough such that it does not interfere with activity of the polypeptide to which it is linked. The tag polypeptide typically is sufficiently unique so that an antibody that specifically binds thereto does not substantially cross-react with epitopes in the polypeptide to which it is linked.

Suitable tag polypeptides generally have at least 5 or 6 amino acid residues and usually between about 8-50 amino acid residues, typically between 9-30 residues. The tags can be linked to one or more chimeric polypeptides in a multimer and permit detection of the multimer or its recovery from a sample or mixture. Such tags are well-known and can be readily synthesized and designed. Exemplary tag polypeptides include those used for affinity purification and include, FLAG tags, His tags, the influenza hemagglutinin (HA) tag polypeptide and its antibody 12CA5, (Field et al. (1988) Mol. Cell. Biol. 8:2159-2165); the c-myc tag and the 8F9, 3C7, 6E10, G4, B7 and 9E10 antibodies thereto (see, e.g., Evan et al. (1985) Molecular and Cellular Biology 5:3610-3616); and the Herpes Simplex virus glycoprotein D (gD) tag and its antibody (Paborsky et al. (1990) Protein Engineering 3:547-553). An antibody used to detect an epitope-tagged antibody is typically referred to herein as a secondary antibody.

As used herein, a label or detectable moiety is a detectable marker (e.g., a fluorescent molecule, chemiluminescent molecule, a bioluminescent molecule, a contrast agent (e.g., a metal), a radionuclide, a chromophore, a detectable peptide, or an enzyme that catalyzes the formation of a detectable product) that can be attached or linked directly or indirectly to a molecule (e.g., an antibody or antigen-binding fragment thereof, such as an anti-EGFR antibody or antigen-binding fragment thereof provided herein) or associated therewith and can be detected in vivo and/or in vitro. The detection method can be any method known in the art, including known in vivo and/or in vitro methods of detection (e.g., imaging by visual inspection, magnetic resonance (MR) spectroscopy, ultrasound signal, X-ray, gamma ray spectroscopy (e.g., positron emission tomography (PET) scanning, single-photon emission computed tomography (SPECT)), fluorescence spectroscopy or absorption). Indirect detection refers to measurement of a physical phenomenon, such as energy or particle emission or absorption, of an atom, molecule or composition that binds directly or indirectly to the detectable moiety (e.g., detection of a labeled secondary antibody or antigen-binding fragment thereof that binds to a primary antibody (e.g., an anti-EGFR antibody or antigen-binding fragment thereof provided herein).

As used herein, “nucleic acid” refers to at least two linked nucleotides or nucleotide derivatives, including a deoxyribonucleic acid (DNA) and a ribonucleic acid (RNA), joined together, typically by phosphodiester linkages. Also included in the term “nucleic acid” are analogs of nucleic acids such as peptide nucleic acid (PNA), phosphorothioate DNA, and other such analogs and derivatives or combinations thereof. Nucleic acids also include DNA and RNA derivatives containing, for example, a nucleotide analog or a “backbone” bond other than a phosphodiester bond, for example, a phosphotriester bond, a phosphoramidate bond, a phosphorothioate bond, a thioester bond, or a peptide bond (peptide nucleic acid). The term also includes, as equivalents, derivatives, variants and analogs of either RNA or DNA made from nucleotide analogs, single (sense or antisense) and double-stranded nucleic acids. Deoxyribonucleotides include deoxyadenosine, deoxycytidine, deoxyguanosine and deoxythymidine. For RNA, the uracil base is uridine.

As used herein, an isolated nucleic acid molecule is one which is separated from other nucleic acid molecules which are present in the natural source of the nucleic acid molecule. An “isolated” nucleic acid molecule, such as a cDNA molecule, can be substantially free of other cellular material, or culture medium when produced by recombinant techniques, or substantially free of chemical precursors or other chemicals when chemically synthesized. Exemplary isolated nucleic acid molecules provided herein include isolated nucleic acid molecules encoding an antibody or antigen-binding fragments provided.

As used herein, “operably linked” with reference to nucleic acid sequences, regions, elements or domains means that the nucleic acid regions are functionally related to each other. For example, nucleic acid encoding a leader peptide can be operably linked to nucleic acid encoding a polypeptide, whereby the nucleic acids can be transcribed and translated to express a functional fusion protein, wherein the leader peptide effects secretion of the fusion polypeptide. In some instances, the nucleic acid encoding a first polypeptide (e.g., a leader peptide) is operably linked to nucleic acid encoding a second polypeptide and the nucleic acids are transcribed as a single mRNA transcript, but translation of the mRNA transcript can result in one of two polypeptides being expressed. For example, an amber stop codon can be located between the nucleic acid encoding the first polypeptide and the nucleic acid encoding the second polypeptide, such that, when introduced into a partial amber suppressor cell, the resulting single mRNA transcript can be translated to produce either a fusion protein containing the first and second polypeptides, or can be translated to produce only the first polypeptide. In another example, a promoter can be operably linked to nucleic acid encoding a polypeptide, whereby the promoter regulates or mediates the transcription of the nucleic acid.

As used herein, “synthetic,” with reference to, for example, a synthetic nucleic acid molecule or a synthetic gene or a synthetic peptide refers to a nucleic acid molecule or polypeptide molecule that is produced by recombinant methods and/or by chemical synthesis methods.

As used herein, the residues of naturally occurring α-amino acids are the residues of those 20 α-amino acids found in nature which are incorporated into protein by the specific recognition of the charged tRNA molecule with its cognate mRNA codon in humans.

As used herein, “polypeptide” refers to two or more amino acids covalently joined. The terms “polypeptide” and “protein” are used interchangeably herein.

As used herein, a “peptide” refers to a polypeptide that is from 2 to about or 40 amino acids in length.

As used herein, an “amino acid” is an organic compound containing an amino group and a carboxylic acid group. A polypeptide contains two or more amino acids. For purposes herein, amino acids contained in the antibodies provided include the twenty naturally-occurring amino acids (Table 3), non-natural amino acids, and amino acid analogs (e.g., amino acids wherein the α-carbon has a side chain). As used herein, the amino acids, which occur in the various amino acid sequences of polypeptides appearing herein, are identified according to their well-known, three-letter or one-letter abbreviations (see Table 3). The nucleotides, which occur in the various nucleic acid molecules and fragments, are designated with the standard single-letter designations used routinely in the art.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3557-59 (1968) and adopted at 37 C.F.R. §§1.821-1.822, abbreviations for amino acid residues are shown in Table 3:

TABLE 3
Table of Correspondence

SYMBOL

| 1-Letter | 3-Letter | AMINO ACID |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Proline |
| K | Lys | Lysine |
| H | His | Histidine |
| Q | Gln | Glutamine |
| E | Glu | Glutamic acid |
| Z | Glx | Glutamic Acid and/or Glutamine |
| W | Trp | Tryptophan |
| R | Arg | Arginine |
| D | Asp | Aspartic acid |
| N | Asn | Asparagine |
| B | Asx | Aspartic Acid and/or Asparagine |
| C | Cys | Cysteine |
| X | Xaa | Unknown or other |

All sequences of amino acid residues represented herein by a formula have a left to right orientation in the conventional direction of amino-terminus to carboxyl-terminus. In addition, the phrase “amino acid residue” is defined to include the amino acids listed in the Table of Correspondence (Table 3), modified, non-natural and unusual amino acids. Furthermore, a dash at the beginning or end of an amino acid residue sequence indicates a peptide bond to a further sequence of one or more amino acid residues or to an amino-terminal group such as NH₂ or to a carboxyl-terminal group such as COOH.

In a peptide or protein, suitable conservative substitutions of amino acids are known to those of skill in the art and generally can be made without altering a biological activity of a resulting molecule. Those of skill in the art recognize that, in general, single amino acid substitutions in non-essential regions of a polypeptide do not substantially alter biological activity (see, e.g., Watson et al., Molecular Biology of the Gene, 4th Edition, 1987, The Benjamin/Cummings Pub. co., p. 224).

Such substitutions can be made in accordance with the exemplary substitutions set forth in Table 4 as follows:

TABLE 4
Exemplary conservative amino acid substitutions

| Original | Conservative |
|---|---|
| residue | substitution |
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with other known conservative or non-conservative substitutions.

As used herein, “naturally occurring amino acids” refer to the 20 L-amino acids that occur in polypeptides.

As used herein, the term “non-natural amino acid” refers to an organic compound that has a structure similar to a natural amino acid but has been modified structurally to mimic the structure and reactivity of a natural amino acid. Non-naturally occurring amino acids thus include, for example, amino acids or analogs of amino acids other than the 20 naturally occurring amino acids and include, but are not limited to, the D-stereoisomers of amino acids. Exemplary non-natural amino acids are known to those of skill in the art, and include, but are not limited to, 2-Aminoadipic acid (Aad), 3-Aminoadipic acid (bAad), β-alanine/β-Amino-propionic acid (Bala), 2-Aminobutyric acid (Abu), 4-Aminobutyric acid/piperidinic acid (4Abu), 6-Aminocaproic acid (Acp), 2-Aminoheptanoic acid (Ahe), 2-Aminoisobutyric acid (Aib), 3-Aminoisobutyric acid (Baib), 2-Aminopimelic acid (Apm), 2,4-Diaminobutyric acid (Dbu), Desmosine (Des), 2,2′-Diaminopimelic acid (Dpm), 2,3-Diaminopropionic acid (Dpr), N-Ethylglycine (EtGly), N-Ethylasparagine (EtAsn), Hydroxylysine (Hyl), allo-Hydroxylysine (Ahyl), 3-Hydroxyproline (3Hyp), 4-Hydroxyproline (4Hyp), Isodesmosine (Ide), allo-Isoleucine (Aile), N-Methylglycine, sarcosine (MeGly), N-Methylisoleucine (MeIle), 6-N-Methyllysine (MeLys), N-Methylvaline (MeVal), Norvaline (Nva), Norleucine (Nle), and Ornithine (Orn).

As used herein, a DNA construct is a single or double stranded, linear or circular DNA molecule that contains segments of DNA combined and juxtaposed in a manner not found in nature. DNA constructs exist as a result of human manipulation, and include clones and other copies of manipulated molecules.

As used herein, a DNA segment is a portion of a larger DNA molecule having specified attributes. For example, a DNA segment encoding a specified polypeptide is a portion of a longer DNA molecule, such as a plasmid or plasmid fragment, which, when read from the 5′ to 3′ direction, encodes the sequence of amino acids of the specified polypeptide.

As used herein, the term polynucleotide means a single- or double-stranded polymer of deoxyribonucleotides or ribonucleotide bases read from the 5′ to the 3′ end. Polynucleotides include RNA and DNA, and can be isolated from natural sources, synthesized in vitro, or prepared from a combination of natural and synthetic molecules. The length of a polynucleotide molecule is given herein in terms of nucleotides (abbreviated “nt”) or base pairs (abbreviated “bp”). The term nucleotides is used for single- and double-stranded molecules where the context permits. When the term is applied to double-stranded molecules it is used to denote overall length and will be understood to be equivalent to the term base pairs. It will be recognized by those skilled in the art that the two strands of a double-stranded polynucleotide can differ slightly in length and that the ends thereof can be staggered; thus all nucleotides within a double-stranded polynucleotide molecule cannot be paired. Such unpaired ends will, in general, not exceed 20 nucleotides in length.

As used herein, production by recombinant means by using recombinant DNA methods means the use of the well-known methods of molecular biology for expressing proteins encoded by cloned DNA.

As used herein, “expression” refers to the process by which polypeptides are produced by transcription and translation of polynucleotides. The level of expression of a polypeptide can be assessed using any method known in art, including, for example, methods of determining the amount of the polypeptide produced from the host cell. Such methods can include, but are not limited to, quantitation of the polypeptide in the cell lysate by ELISA, Coomassie blue staining following gel electrophoresis, Lowry protein assay and Bradford protein assay.

As used herein, a “host cell” is a cell that is used to receive, maintain, reproduce and/or amplify a vector. A host cell also can be used to express the polypeptide encoded by the vector. The nucleic acid contained in the vector is replicated when the host cell divides, thereby amplifying the nucleic acids.

As used herein, a “vector” is a replicable nucleic acid from which one or more heterologous proteins, can be expressed when the vector is transformed into an appropriate host cell. Reference to a vector includes those vectors into which a nucleic acid encoding a polypeptide or fragment thereof can be introduced, typically by restriction digest and ligation. Reference to a vector also includes those vectors that contain nucleic acid encoding a polypeptide, such as a modified anti-EGFR antibody. The vector is used to introduce the nucleic acid encoding the polypeptide into the host cell for amplification of the nucleic acid or for expression/display of the polypeptide encoded by the nucleic acid. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well-known to those of skill in the art. A vector also includes “virus vectors” or “viral vectors.” Viral vectors are engineered viruses that are operatively linked to exogenous genes to transfer (as vehicles or shuttles) the exogenous genes into cells.

As used herein, an “expression vector” includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well-known to those of skill in the art and include those that are replicable in eukaryotic cells and/or prokaryotic cells and those that remain episomal or those which integrate into the host cell genome.

As used herein, “primary sequence” refers to the sequence of amino acid residues in a polypeptide or the sequence of nucleotides in a nucleic acid molecule.

As used herein, “sequence identity” refers to the number of identical or similar amino acids or nucleotide bases in a comparison between a test and a reference polypeptide or polynucleotide. Sequence identity can be determined by sequence alignment of nucleic acid or protein sequences to identify regions of similarity or identity. For purposes herein, sequence identity is generally determined by alignment to identify identical residues. The alignment can be local or global. Matches, mismatches and gaps can be identified between compared sequences. Gaps are null amino acids or nucleotides inserted between the residues of aligned sequences so that identical or similar characters are aligned. Generally, there can be internal and terminal gaps. When using gap penalties, sequence identity can be determined with no penalty for end gaps (e.g., terminal gaps are not penalized). Alternatively, sequence identity can be determined without taking into account gaps as the number of identical positions/length of the total aligned sequence×100.

As used herein, a “global alignment” is an alignment that aligns two sequences from beginning to end, aligning each letter in each sequence only once. An alignment is produced, regardless of whether or not there is similarity or identity between the sequences. For example, 50% sequence identity based on “global alignment” means that in an alignment of the full sequence of two compared sequences each of 100 nucleotides in length, 50% of the residues are the same. It is understood that global alignment also can be used in determining sequence identity even when the length of the aligned sequences is not the same. The differences in the terminal ends of the sequences will be taken into account in determining sequence identity, unless the “no penalty for end gaps” is selected. Generally, a global alignment is used on sequences that share significant similarity over most of their length. Exemplary algorithms for performing global alignment include the Needleman-Wunsch algorithm (Needleman et al. J. Mol. Biol. 48: 443 (1970). Exemplary programs for performing global alignment are publicly available and include the Global Sequence Alignment Tool available at the National Center for Biotechnology Information (NCBI) website (ncbi.nlm.nih.gov/), and the program available at deepc2.psi.iastate.edu/aat/align/align.html.

As used herein, a “local alignment” is an alignment that aligns two sequence, but only aligns those portions of the sequences that share similarity or identity. Hence, a local alignment determines if sub-segments of one sequence are present in another sequence. If there is no similarity, no alignment will be returned. Local alignment algorithms include BLAST or Smith-Waterman algorithm (Adv. Appl. Math. 2: 482 (1981)). For example, 50% sequence identity based on “local alignment” means that in an alignment of the full sequence of two compared sequences of any length, a region of similarity or identity of 100 nucleotides in length has 50% of the residues that are the same in the region of similarity or identity.

For purposes herein, sequence identity can be determined by standard alignment algorithm programs used with default gap penalties established by each supplier. Default parameters for the GAP program can include: (1) a unary comparison matrix (containing a value of 1 for identities and 0 for non-identities) and the weighted comparison matrix of Gribskov et al. Nucl. Acids Res. 14: 6745 (1986), as described by Schwartz and Dayhoff, eds., Atlas of Protein Sequence and Structure, National Biomedical Research Foundation, pp. 353-358 (1979); (2) a penalty of 3.0 for each gap and an additional 0.10 penalty for each symbol in each gap; and (3) no penalty for end gaps. Whether any two nucleic acid molecules have nucleotide sequences or any two polypeptides have amino acid sequences that are at least 80%, 85%, 90%, 95%, 96%, 97%, 98% or 99% “identical,” or other similar variations reciting a percent identity, can be determined using known computer algorithms based on local or global alignment (see e.g., wikipedia.org/wiki/Sequence_alignment_software, providing links to dozens of known and publicly available alignment databases and programs). Generally, for purposes herein sequence identity is determined using computer algorithms based on global alignment, such as the Needleman-Wunsch Global Sequence Alignment tool available from NCBI/BLAST (blast.ncbi.nlm.nih.gov/Blast.cgi?CMD=Web&Page_TYPE=BlastHome); LAlign (William Pearson implementing the Huang and Miller algorithm (Adv. Appl. Math. (1991) 12:337-357)); and program from Xiaoqui Huang available at deepc2.psi.iastate.edu/aat/align/align.html. Typically, the full-length sequence of each of the compared polypeptides or nucleotides is aligned across the full-length of each sequence in a global alignment. Local alignment also can be used when the sequences being compared are substantially the same length.

Therefore, as used herein, the term “identity” represents a comparison or alignment between a test and a reference polypeptide or polynucleotide. In one non-limiting example, “at least 90% identical to” refers to percent identities from 90 to 100% relative to the reference polypeptide or polynucleotide. Identity at a level of 90% or more is indicative of the fact that, assuming for exemplification purposes a test and reference polypeptide or polynucleotide length of 100 amino acids or nucleotides are compared, no more than 10% (i.e., 10 out of 100) of amino acids or nucleotides in the test polypeptide or polynucleotide differ from those of the reference polypeptide. Similar comparisons can be made between a test and reference polynucleotides. Such differences can be represented as point mutations randomly distributed over the entire length of an amino acid sequence or they can be clustered in one or more locations of varying length up to the maximum allowable, e.g., 10/100 amino acid difference (approximately 90% identity). Differences also can be due to deletions or truncations of amino acid residues. Differences are defined as nucleic acid or amino acid substitutions, insertions or deletions. Depending on the length of the compared sequences, at the level of homologies or identities above about 85-90%, the result can be independent of the program and gap parameters set; such high levels of identity can be assessed readily, often without relying on software.

As used herein, a disulfide bond (also called an S—S bond or a disulfide bridge) is a single covalent bond derived from the coupling of thiol groups. Disulfide bonds in proteins are formed between the thiol groups of cysteine residues, and stabilize interactions between polypeptide domains, such as antibody domains.

As used herein, “coupled” or “conjugated” means attached via a covalent or noncovalent interaction.

As used herein, the phrase “conjugated to an antibody” or “linked to an antibody” or grammatical variations thereof, when referring to the attachment of a moiety to an antibody or antigen-binding fragment thereof, such as a diagnostic or therapeutic moiety, means that the moiety is attached to the antibody or antigen-binding fragment thereof by any known means for linking peptides, such as, for example, by production of fusion protein by recombinant means or post-translationally by chemical means. Conjugation can employ any of a variety of linking agents to effect conjugation, including, but not limited to, peptide or compound linkers or chemical cross-linking agents.

As used herein “auristatin drug moiety” refers to the substructure of an antibody-drug-conjugate that has the structure of an aurastin derivative. Aurastins are a class of synthetic molecules that interfere with microtubule dynmaics, GTP hydrolysis and nuclear and cellular division. Exemplary auristatin embodiments include N-terminally and C-terminally linked monomethylauristatin drug moieties MMAE and MMAF (Senter et al. (2004) “Proceedings of the American Association for Cancer Research,” Volume 45, Abstract Number 623, and presented Mar. 28, 2004; U.S. Publication No. 2011/0020343). The synthesis and structure of exemplary auristatin derivatives are described in U.S. Patent Application Publication Nos. 2003-0083263, 2005-0238649 and 2005-0009751; International Patent Publication No. WO 04/010957, International Patent Publication No. WO 02/088172, and U.S. Pat. Nos. 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, each of which is incorporated by reference herein in its entirety.

As used herein, “Maytansinoid drug moiety” means the substructure of an antibody-drug conjugate that has the structure of a maytansine compound. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and maytansinol analogs have been reported. See U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,234; 4,362,663; and 4,371,533, and Kawai et al (1984) Chem. Pharm. Bull. 3341-3351).

A “free cysteine amino acid” refers to a cysteine amino acid residue that has a thiol functional group (—SH), and is not paired as an intramolecular or intermolecular disulfide bridge. It can be engineered into a parent antibody.

As used herein, “Linker”, “Linker Unit”, or “link” means a peptide or chemical moiety containing a chain of atoms that covalently attaches an antibody to a drug moiety or therapeutic moiety.

As used herein, “Antibody-dependent cell-mediated cytotoxicity” and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, (1991) Annu. Rev. Immunol, 9:457-92. To assess ADCC activity of a molecule of interest, an in vitro ADCC assay may be performed (U.S. Pat. No. 5,500,362; U.S. Pat. No. 5,821,337). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al (1998) PNAS (USA), 95:652-656.

As used herein “therapeutic activity” refers to the in vivo activity of a therapeutic polypeptide. Generally, the therapeutic activity is the activity that is associated with treatment of a disease or condition. For example, the therapeutic activity of an anti-EGFR antibody includes inhibitory activities on EGFR phosphorylation, signaling and cell growth, and in particular inhibitory activities on tumor cell growth. Therapeutic activity of a modified polypeptide can be any level of percentage of therapeutic activity of the unmodified polypeptide, including but not limited to, 1% of the activity, 2%, 3%, 4%, 5%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more of therapeutic activity compared to the unmodified polypeptide.

As used herein, the term “assessing” is intended to include quantitative and qualitative determination in the sense of obtaining an absolute value for the activity of a protein, such as a modified anti-EGFR antibody, or an antigen binding fragment thereof, present in the sample, and also of obtaining an index, ratio, percentage, visual, or other value indicative of the level of the activity. Assessment can be direct or indirect.

As used herein, “disease or disorder” refers to a pathological condition in an organism resulting from cause or condition including, but not limited to, infections, acquired conditions, genetic conditions, and characterized by identifiable symptoms.

As used herein, “EGFR-associated disease or condition” or “conditions responsive to treatment with an anti-EGFR antibody,” refers to any disease or condition that is associated with or caused by aberrant EGFR signaling or overexpression of EGFR. Such diseases and conditions are known in the art, and examples of such are described herein. For example, EGFR-associated diseases or conditions or conditions responsive to treatment with an anti-EGFR antibody include cancers, such as, but not limited to, colorectal cancer, squamous cell cancer of the head and neck and non-small-cell lung cancer.

As used herein, “treating” a subject with a disease or condition means that the subject's symptoms are partially or totally alleviated, or remain static following treatment. Hence treatment encompasses prophylaxis, therapy and/or cure. Prophylaxis refers to prevention of a potential disease and/or a prevention of worsening of symptoms or progression of a disease. Treatment also encompasses any pharmaceutical use of any antibody or antigen-binding fragment thereof provided or compositions provided herein.

As used herein, “prevention” or prophylaxis, and grammatically equivalent forms thereof, refers to methods in which the risk of developing disease or condition is reduced.

As used herein, a “pharmaceutically effective agent” includes any therapeutic agent or bioactive agents, including, but not limited to, for example, anesthetics, vasoconstrictors, dispersing agents, conventional therapeutic drugs, including small molecule drugs and therapeutic proteins.

As used herein, a “therapeutic effect” means an effect resulting from treatment of a subject that alters, typically improves or ameliorates the symptoms of a disease or condition or that cures a disease or condition.

As used herein, a “therapeutically effective amount” or a “therapeutically effective dose” refers to the quantity of an agent, compound, material, or composition containing a compound that is at least sufficient to produce a therapeutic effect following administration to a subject. Hence, it is the quantity necessary for preventing, curing, ameliorating, arresting or partially arresting a symptom of a disease or disorder.

As used herein, “therapeutic efficacy” refers to the ability of an agent, compound, material, or composition containing a compound to produce a therapeutic effect in a subject to whom the an agent, compound, material, or composition containing a compound has been administered.

As used herein, a “prophylactically effective amount” or a “prophylactically effective dose” refers to the quantity of an agent, compound, material, or composition containing a compound that when administered to a subject, will have the intended prophylactic effect, e.g., preventing or delaying the onset, or reoccurrence, of disease or symptoms, reducing the likelihood of the onset, or reoccurrence, of disease or symptoms, or reducing the incidence of viral infection. The full prophylactic effect does not necessarily occur by administration of one dose, and can occur only after administration of a series of doses. Thus, a prophylactically effective amount can be administered in one or more administrations.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, “Prodrug” is a precursor or derivative form of a pharmaceutically active substance that is less cytotoxic to tumor cells compared to the parent drug and is capable of being enzymatically activated or converted into the more active parent form(see, e.g., Wilman, 1986, Biochemical Society Transactions, 615th Meeting Belfast, 14:375-382; and Stella et al., “Prodrugs: A Chemical Approach to Targeted Drug Delivery,” Directed Drug Delivery, Borchardt et al., (ed.): 247-267, Humana Press, 1985.)

As used herein, an “anti-cancer agent” refers to any agent that is destructive or toxic to malignant cells and tissues. For example, anti-cancer agents include agents that kill cancer cells or otherwise inhibit or impair the growth of tumors or cancer cells. Exemplary anti-cancer agents are chemotherapeutic agents.

As used herein, an “anti-angiogenic agent” or “angiogenesis inhibitor” is a compound that blocks, or interferes with, the development of blood vessels.

As used herein, a “hyperproliferative disease” is a condition caused by excessive growth of non-cancer cells that express a member of the EGFR family of receptors.

As used herein, the term “subject” refers to an animal, including a mammal, such as a human being.

As used herein, a patient refers to a human subject.

As used herein, animal includes any animal, such as, but are not limited to primates including humans, gorillas and monkeys; rodents, such as mice and rats; fowl, such as chickens; ruminants, such as goats, cows, deer, sheep; pigs and other animals. Non-human animals exclude humans as the contemplated animal. The polypeptides provided herein are from any source, animal, plant, prokaryotic and fungal. Most polypeptides are of animal origin, including mammalian origin.

As used herein, a “composition” refers to any mixture. It can be a solution, suspension, liquid, powder, paste, aqueous, non-aqueous or any combination thereof

As used herein, a stabilizing agent refers to compound added to the formulation to protect either the antibody or conjugate, such as under the conditions (e.g. temperature) at which the formulations herein are stored or used. Thus, included are agents that prevent proteins from degradation from other components in the compositions. Exemplary of such agents are amino acids, amino acid derivatives, amines, sugars, polyols, salts and buffers, surfactants, inhibitors or substrates and other agents as described herein.

As used herein, a “combination” refers to any association between or among two or more items. The combination can be two or more separate items, such as two compositions or two collections, a mixture thereof, such as a single mixture of the two or more items, or any variation thereof. The elements of a combination are generally functionally associated or related.

As used herein, combination therapy refers to administration of two or more different therapeutics, such as an anti-EGFR antibody (or antigen binding fragment thereof) and one or more therapeutics. The different therapeutic agents can be provided and administered separately, sequentially, intermittently, or can be provided in a single composition.

As used herein, a kit is a packaged combination that optionally includes other elements, such as additional reagents and instructions for use of the combination or elements thereof, for a purpose including, but not limited to, activation, administration, diagnosis, and assessment of a biological activity or property.

As used herein, a “unit dose form” refers to physically discrete units suitable for human and animal subjects and packaged individually as is known in the art.

As used herein, a “single dosage formulation” refers to a formulation for direct administration.

As used herein, a multi-dose formulation refers to a formulation that contains multiple doses of a therapeutic agent and that can be directly administered to provide several single doses of the therapeutic agent. The doses can be administered over the course of minutes, hours, weeks, days or months. Multidose formulations can allow dose adjustment, dose-pooling and/or dose-splitting. Because multi-dose formulations are used over time, they generally contain one or more preservatives to prevent microbial growth.

As used herein, an “article of manufacture” is a product that is made and sold. As used throughout this application, the term is intended to encompass any of the compositions provided herein contained in articles of packaging.

As used herein, a “fluid” refers to any composition that can flow. Fluids thus encompass compositions that are in the form of semi-solids, pastes, solutions, aqueous mixtures, gels, lotions, creams and other such compositions.

As used herein, an isolated or purified polypeptide or protein (e.g., an isolated antibody or antigen-binding fragment thereof) or biologically-active portion thereof (e.g., an isolated antigen-binding fragment) is substantially free of cellular material or other contaminating proteins from the cell or tissue from which the protein is derived, or substantially free from chemical precursors or other chemicals when chemically synthesized. Preparations can be determined to be substantially free if they appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification does not detectably alter the physical and chemical properties, such as enzymatic and biological activities, of the substance. Methods for purification of the compounds to produce substantially chemically pure compounds are known to those of skill in the art. A substantially chemically pure compound, however, can be a mixture of stereoisomers. In such instances, further purification might increase the specific activity of the compound. As used herein, a “cellular extract” or “lysate” refers to a preparation or fraction which is made from a lysed or disrupted cell.

As used herein, a “control” refers to a sample that is substantially identical to the test sample, except that it is not treated with a test parameter, or, if it is a plasma sample, it can be from a normal volunteer not affected with the condition of interest. A control also can be an internal control.

As used herein, the singular forms “a,” “an” and “the” include plural referents unless the context clearly dictates otherwise. Thus, for example, reference to a polypeptide, comprising “an immunoglobulin domain” includes polypeptides with one or a plurality of immunoglobulin domains.

As used herein, the term “or” is used to mean “and/or” unless explicitly indicated to refer to alternatives only or the alternatives are mutually exclusive.

As used herein, ranges and amounts can be expressed as “about” a particular value or range. About also includes the exact amount. Hence “about 5 amino acids” means “about 5 amino acids” and also “5 amino acids.”

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

For clarity of disclosure, and not by way of limitation, the detailed description is divided into the subsections that follow.

### B. EGFR AND ANTI-EGFR ANTIBODIES

Provided herein are anti-epidermal growth factor receptor (EGFR) antibodies that exhibit greater binding activity under acidic pH conditions and/or elevated lactate levels (e.g., present in a tumor microenvironment) than under neutral pH conditions/normal lactate levels (e.g., present in skin dermis). Anti-EGFR antibodies are known and approved for various indications, including metastatic colorectal cancer (mCRC), squamous cell carcinoma of the head and neck (SCCHN) and non-small cell lung cancer (NSCLC), pancreatic cancer, breast cancer, gastric cancer, ovarian cancer, rectal cancer, bladder cancer, and other solid tumors. Anti-EGFR antibodies include, but are not limited to, Erbitux® (cetuximab, C225 or IMC-C225), 11F8 by Zhu (WO 2005/090407), EMD 72000 (matuzumab), Vectibix™ (panitumumab; ABX-EGF), TheraCIM (nimotuzumab), and Hu-Max-EGFR (zalutumumab). These antibodies, however, exhibit substantially similar binding activity for EGFR under varied pH conditions so that their activity is not tumor-specific, thereby resulting in unwanted activity at non-target sites such as the skin. Thus, when administered to subjects, these therapeutic antibodies result in adverse side effects to the subjects (Eng C. (2009) Nat. Rev. Clin. Oncol., 6:207-218). This has limited their use.

For example, anti-EGFR antibodies are associated with significant and characteristic adverse events including skin toxicities and digestive disturbances (including nausea, vomiting, diarrhea), that often lead to interruption of dosing and discontinuation of treatment. For example, EGFR, is highly expressed in pre-keratinocytes and basal cells of the skin. Blockade of EGFR signaling in the skin precursors by anti-EGFR antibodies leads to skin precursor growth inhibition, apoptosis and inflammation. This can result in skin toxicity, such as a rash and other skin lesions. In particular, existing anti-EGFR antibodies (e.g., cetuximab, panitumumab) exhibit high toxicity with up to 80% attributed to skin-related toxicity, including 25% that is Grade 3-4 (Cunningham et al. (2004) NEJM, 351:337). In particular, skin lesions can include rash with itchy erythematous follicular papules that can evolve into pustules.

As a therapeutic, the activity of anti-EGFR antibodies is principally targeted to the tumor environment, which exhibits an acidic pH and elevated lactate levels, e.g., between 10-15 mM lactate. In contrast, the dermis, which is where many side effects are localized, exhibits a neutral pH and normal lactate levels. It is found herein that side effects can be reduced by providing antibodies that exhibit increased activity at targeted disease tissue, such as the tumor, but decreased activity at non-disease tissues or organs, in particular tissue sites (e.g., basal layer of skin or dermis) associated with adverse events. The differences in conditions that characterize solid tumors, such as low pH and hypoxia, can be leveraged to provide antibodies that are more active in the diseased microenvironment of the tumor. Hence, provided herein are modified anti-EGFR antibodies that are conditionally active in the tumor microenvironment and exhibit altered activity or increased activity under conditions present in the tumor microenvironment compared to normal tissues. For example, the antibodies provided herein are more active at low pH and/or high lactate, than at neutral pH or low lactate. As a consequence of this altered activity, subjects treated with the antibodies have fewer and/or reduced side effects.

In particular, it is found that modified anti-EGFR antibodies containing an amino acid replacement in the variable heavy chain with a negatively charged amino acid (e.g., Asp or Glu) at a position corresponding to position 104 with reference to the variable heavy chain set forth in SEQ ID NO: 2 or 7 exhibit increased activity, for example binding activity, at lower or acidic pH, for example, pH 6.0 to 6.5, inclusive, such as the acidic pH environment of the tumor, than at neutral pH (e.g., pH 7.4). Modified anti-EGFR antibodies containing the amino acid replacement to Glu (E), however, are shown herein to exhibit substantially weaker or lower binding activity than antibodies containing the amino acid replacement Asp (D) at neutral pH (e.g., pH 7.4). This difference could be due to the presence of an extra —CH2 group that affects the acidity of the molecule. By virtue of the decreased binding at neutral pH, modified anti-EGFR antibodies provided herein containing an amino acid replacement in the variable heavy chain with the negatively charged amino acid Glu (E) at a position corresponding to position 104 with reference to the variable heavy chain set forth in SEQ ID NO: 2 or 7 (e.g., Y104E) exhibit improved acidic pH-binding selectivity, and thereby improved tumor-targeted selectivity where activity is desired. Such modified anti-EGFR antibodies also can exhibit increased activity, for example binding activity, at increased lactate concentrations, such as at concentrations between 15 and 20 mM lactate. For example, the anti-EGFR antibodies provided herein bind with increased activity, such as binding activity, at both reduced pH (e.g., acidic pH 6.0 to 6.5, inclusive) and elevated lactate levels (e.g., 15 mM to 20 mM lactate). The anti-EGFR antibodies provided herein exhibit altered activity such that they confer reduced or fewer side effects when administered.

1. EGFR

Epidermal growth factor receptor (EGFR; also known as receptor tyrosine-protein kinase erbB-1, ErbB-1, HER1) (Uniprot Accession No. P00533; SEQ ID NO: 43) is a 170 kDa Type I glycoprotein. EGFR is a member of the ErbB family of receptor tyrosine kinases, which includes HER2/c-neu (ErbB-2), Her3 (ErbB-3) and Her4 (ErbB-4). EGFR exists on cell surfaces and contains three domains, including an extracellular ligand-binding domain, an intracellular tyrosine kinase domain and a transmembrane lipophilic segment. In addition to their presence on a tumor cells, epidermal growth factor receptors are ubiquitous, distributed randomly on the surface of normal cells, excluding hematopoietic cells and cells of epidermal origin.

EGFR is a tyrosine kinase growth factor receptor involved in signaling cascades important for cell growth, proliferation, survival and motility. EGFR activity is stimulated or activated by binding of endogenous ligands such as epidermal growth factor (EGF), as well as other endogenous EGF-like ligands including TGF-α, amphiregulin, heparin-binding EGF (HB-EGF) and betacellulin. Upon ligand binding, the ligand-EGFR complex undergoes dimerization and internalization into the cell. EGFR can homodimerize with other monomeric EGFR molecules, or alternatively, heterodimerize with another HER receptor, such as HER2, ErbB-3 or ErbB-4. EGFR dimerization leads to autophosphorylation of tyrosine residues in the cytoplasmic tail of EGFR and activates intrinsic intracellular protein-tyrosine kinase activity. The EGFR phosphotyrosine residues act as docking sites for downstream effectors such as adaptor molecules and enzymes leading to initiation of a variety of signal transduction pathways, including mitogen-activated protein kinase (MAPK), Akt/phosphatidylinositol-3-OH kinase (PI3K) and c-Jun N-terminal kinases (JNK), thereby regulating a variety of mitogenic mechanisms involved in DNA synthesis, cell proliferation, cell migration, cell survival and cell adhesion.

EGFR is important in regulating cell survival and apoptosis, angiogenesis, cell motility and metastasis (Herbst et al. (2001) Expert Opin. Biol. Ther. 1(4):719-732). EGFR activation is associated with significant upregulation of secretion of vascular endothelial growth factor, a stimulator of tumor angiogenesis (Petit at al. (1997) Am J Pathol 151:1523-1530). Aberrant EGFR signaling and EGFR overexpression have been observed in various cancers and are correlated with poor prognosis and elevated risk of invasive or metastatic disease (Herbst et al. (2001) Expert Opin. Biol. Ther. 1(4):719-732). For example, deregulation of EGFRs have been observed in a variety of solid human tumors, including glioma and colon, head and neck, pancreatic, non-small cell lung, breast, renal, ovarian, and bladder carcinomas (Herbst and Hong (2002) Seminars in Oncology 29(5) Suppl. 14: 18-30). As such, EGFR is an attractive target for anti-cancer therapeutics.

2. Anti-EGFR Antibodies and Side Effects

Therapeutic agents that target and inhibit aberrant EGFR signaling include anti-EGFR antibodies. Anti-EGFR antibodies bind EGFR, thereby inhibiting the binding of ligands, such as EGF, to the extracellular ligand binding domain of EGF and preventing receptor dimerization, autophosphorylation, and resulting signal transduction events. Hence, anti-EGFR antibodies can be effective therapeutics by blocking EGFR-mediated cell signaling and cell growth. Anti-EGFR antibodies are known in the art and many are in clinical development or approved for treatment of cancer. Cetuximab, marketed by ImClone under the trade name Erbitux®, is described in U.S. Pat. Nos. 4,943,533 and 7,060,808, including humanized form. Panitumumab, marketed by Abgenix under the trade name Vectibix, is described in U.S. Pat. No. 6,235,883. Zalutumumab (HuMax-EGFr), developed by Genmab, is described in WO 02/100348 and WO 2004/056847. Cetuximab, Panitumumab, and Zalutumumab bind the same epitope on EGFR. Further monoclonal anti-EGFR antibodies include, but are not limited to, Nimotuzumab (TheraCIM hR3; U.S. Pat. No. 5,891,996 and U.S. Pat. No. 6,506,883); ICR62 (The Institute of Cancer Research; WO 95/20045); mAb806 (Ludwig Institute of Cancer Research; WO 02/092771); and Matuzumab (EMD72000, Merck-Serono; WO 02/66058, WO 92/15683).

Anti-EGFR antibodies, however, cannot distinguish between EGF receptors on the surface of cancer cells and normal cells, and general inhibition of EGFR signaling can result in adverse side effects. For example, EGFR is widely distributed throughout epithelial tissues, and treatments employing many EGFR inhibitors exhibit skin toxicity (Herbst and Hong (2002) Seminars in Oncology 29(5) Suppl. 14: 18-30). In human skin, EGFR is expressed in basal keratinocytes and can stimulate epidermal growth, inhibit differentiation, and accelerate wound healing (Lacouture and Melosky (2007) Skin Therapy Lett. 12, 1-5; Nanney et al. (1990) J. Invest. Dermatol 94(6):742-748; Lacouture, M. E. (2006) Nat Rev Cancer 6:803-812) Inhibition of EGFR function can impair growth and migration of keratinocytes, and result in inflammatory chemokine expression, resulting in rashes (Lacouture, M. E. (2006) Nat Rev Cancer 6:803-812). Increased apoptosis of keratinocytes upon treatment with EGFR inhibitors is correlated with onset of rash in subjects treated with the EGFR inhibitors (Lacouture, M. E. (2006) Nat Rev Cancer 6:803-812). Keratinocytes are located in the stratum basale, the deepest layer of the skin, which has a pH between 7.0 and 7.2. The blood vessels in the dermis provide nourishment and waste removal for the epidermis, thus making the epidermis, in particular the stratum basale, most susceptible to systemically circulated anti-EGFR therapies.

The most common side effects associated with anti-EGFR antibodies, such as cetuximab, are dermatologic reactions, which are seen in 45-100% of patients (Le and Perez-Soler (2009) Target Oncol 4:107-119). Common dermatologic reactions include, acneiform rash, papulopustular rash, hair growth abnormalities, dry and itchy skin and periungual inflammation with tenderness (Eng (2009) Nat Rev Clin Oncol 6:207-218; Monti et al. (2007) Int J Biol Markers 22:S53-S61; Saif and Kim (2007) Expert Opin Drug Saf 6:175-182). Additional dermatologic reactions include telangiectasia, hyperpigmentation, pruritus without rash, erythema and oral aphthae (Eng (2009) Nat Rev Clin Oncol 6:207-218). Cetuximab elicits an immune response in about 5-15% of patients, with some patients reporting severe anaphylactic reactions (Chung et al. (2008) N Engl J Med 358:1109-1117). These hypersensitivity reactions have been linked to galactose-alpha-1,3-galactose oligosaccharides on cetuximab that induce the production of IgG antibodies (Chung et al. (2008) N Engl J Med 358:1109-1117). Further side effects include pulmonary toxicities, including dyspnea, cough, wheezing, pneumonia, hypoxemia, respiratory insufficiency/failure, pulmonary embolus, pleural effusion and non-specific respiratory disorders (Hoag et al. (2009) J Experimental & Clinical Cancer Research 28:113). Other side effects include fever, chills, asthenia/malaise, mucosal surface problems, nausea, gastrointestinal problems, abdominal pain, headache and hypomagnesemia (Eng (2009) Nat Rev Clin Oncol 6:207-218; Fakih and Vincent, (2010) Curr. Oncol. 17(S1):S18-S30; Int. Pat. No. WO2011059762).

The modified anti-EGFR antibodies provided herein exhibit selectivity for binding to tumor cells compared to non-tumor cell targets, such as basal keratinocytes and other basal cell. Hence, the modified anti-EGFR antibodies can result in reduced side effects when administered to patients compared to currently available anti-EGFR antibodies, including eliminating, minimizing or reducing systemic side effects, including dermal toxicities, while retaining their ability to block EGFR signaling. They also permit dosings to achieve increased efficacy compared to existing therapeutics.

3. Cetuximab

Included among the modified anti-EGFR antibodies provided herein are antibodies that are modified (e.g., contain amino acid replacement with a Glu (E) at a position corresponding to position 104 in the variable heavy chain) compared to the anti-EGFR antibody Cetuximab, antigen-binding fragments thereof or variants thereof (e.g., a humanized form of cetuximab, e.g., Hu225 or H225). Cetuximab (also known as C225 or IMC-C225) is a mouse/human chimeric IgG1 monoclonal antibody that binds to human epidermal growth factor receptor. Cetuximab was derived from M225, which was identified using EGFR from human A431 epidermoid carcinoma cells as an immunogen (Gill et al. (1984) J Biol Chem 259:7755-7760; Sato et al., (1983) Mol Biol Med 1:511-529; Masui et al., (1984) Cancer Res 44:1002-1007; Kawamoto et al. (1983) Proc Natl Acad Sci USA 80:1337-1341). M225 inhibits binding of the epidermal growth factor to the EGF receptor and is an antagonist of in vivo EGF-stimulated tyrosine kinase activity. (Gill et al. (1984) J Biol Chem 259:7755-7760).

a. Structure

Cetuximab is a full-length mouse/human chimeric IgG1 antibody. A full-length antibody contains four polypeptide chains, two identical heavy (H) chains (each usually containing about 440 amino acids) and two identical light (L) chains (each containing about 220 amino acids). The light chains exist in two distinct forms called kappa (κ) and lambda (λ). Each chain is organized into a series of domains organized as immunoglobulin (Ig) domains. An Ig domain is characterized by a structure called the Ig fold, which contains two beta-pleated sheets, each containing anti-parallel beta strands connected by loops. The two beta sheets in the Ig fold are sandwiched together by hydrophobic interactions and a conserved intra-chain disulfide bond. The plurality of Ig domains in the antibody chains are organized into a variable (V) and constant (C) region domains.

The variable domains confer antigen-specificity to the antibody through three portions called complementarity determining regions (CDRs) or hypervariable (HV) regions. The CDR regions are precisely defined and universally numbered in antibodies (see e.g., Kabat, E. A. et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; AbM (Martin et al. (1989) Proc Natl Acad Sci USA 86:9268-9272; Martin et al. (1991) Methods Enzymol 203:121-153; Pederson et al. (1992) Immunomethods 1:126). Together, the three heavy chain CDRs and the three light chain CDRs make up an antigen-binding site (antibody combining site) of the antibody, which physically interacts with cognate antigen and provides the specificity of the antibody.

The constant region promotes activation of complement and effector cells. Like CDR regions, constant regions are precisely defined and universally numbered in antibodies using EU index and Kabat numbering schemes (see e.g., Kabat, E. A. et al. (1991) Sequences of Proteins of Immunological Interest, Fifth Edition, U.S. Department of Health and Human Services, NIH Publication No. 91-3242).

Light chains have two domains, corresponding to the C region (C_{L}) and the V region (V_{L}). Heavy chains have four domains, the V region (V_{H}) and three or four domains in the C region (C_{H}1, C_{H}2, C_{H}3 and C_{H}4), and, in some cases, hinge region. Each heavy chain is linked to a light chain by a disulfide bond, and the two heavy chains are linked to each other by disulfide bonds. Linkage of the heavy chains is mediated by a flexible region of the heavy chain, known as the hinge region.

Cetuximab (also called C225) is a human-mouse chimeric antibody that contains variable regions from mouse monoclonal antibody 225 (M225) and a human IgG1 constant region. Antibody M225 is described in U.S. Pat. No. 4,494,533, and can be produced from the hybridoma cell line deposited with the American Type Culture Collection (ATCC) as Accession Number HB 11935. The chimeric form was developed to replace the non-human constant region of M225 with the human IgG1 constant region (see e.g., Prewett et al. (1996) J. Immunother. Emphasis Tumor Immunol., 19:419-27). C225 is commercially known as Erbitux® (cetuximab) and is marketed by ImClone and Bristol-Myers Squibb in the United States, and elsewhere by Merck KgaA. Erbitux® was approved by the FDA in March 2006 for use in combination with radiation therapy for treating squamous cell carcinoma of the head and neck (SCCHN) or as a single agent in patients who have had prior platinum-based therapy. Erbitux® is also indicated for treatment of metastatic colon cancer in combination with irinotecan (Camptosar®), a DNA topoisomerase blocker.

Cetuximab is reported to be composed of 4 polypeptide chains, including 2 identical heavy chains of 449 amino acids each (e.g., set forth in SEQ ID NO: 12), and 2 identical light chains of 214 amino acids each (e.g., set forth in SEQ ID NO: 13) (see IMGT Acc. No. 7906). The variable regions, corresponding to the variable regions of M225, are set forth as amino acid residues 1-119 of SEQ ID NO: 12 (variable heavy chain, set forth in SEQ ID NO: 2) and as amino acid residues 1-107 of SEQ ID NO: 13 (variable light chain, set forth as SEQ ID NO: 4). C225 contains a human IgG1 heavy chain constant region set forth as amino acid residues 120-449 of SEQ ID NO: 12 (set forth in SEQ ID NO: 23) containing human constant domains C_{H}1-C_{H}2-hinge-C_{H}3, including C_{H}1 (amino acid residues 120-217 of SEQ ID NO: 12), a hinge region (amino acid residues 218-232 of SEQ ID NO: 12), C_{H}2 (amino acid residues 233-342 of SEQ ID NO: 12) and C_{H}3 (amino acid residues 343-449 of SEQ ID NO: 12). C225 also contains a human Cκ light chain constant region set forth as amino acid residues 108-213 of SEQ ID NO: 13 (set forth as SEQ ID NO: 34).

It is understood that some variation exists in reported and generated sequences of

Cetuximab, e.g., due to sequencing or cloning artifacts or other variations in the generated sequence. For example, various sequence versions of Cetuximab are described in the literature (see, e.g., U.S. Pat. No. 7,060,808; U.S. Publ. Nos. US 2011-0117110 and US 2013-0266579; International Published PCT Appl. No. WO2004085474; GenBank Accession No. CAH61633; DrugBank Acc. No. DB00002; IMGT Acc. No. 7906). Table 5 sets forth exemplary reference Cetuximab sequences that differ in only a few amino acid residues in non-CDR regions of the heavy chain and/or light chain (see also FIGS. 1A and 1B).

With respect to the exemplary reference sequences set forth in Table 5, the heavy chain is composed of a mouse variable domain (V_{H}, amino acid residues 1-119 of SEQ ID NO: 1, 5, 6 or 12, set forth in SEQ ID NO: 2 or 7) and a light chain composed of a mouse variable domain (V_{L}, amino acid residues 1-107 of SEQ ID NO: 3, 8, 10 or 13, set forth in SEQ ID NO: 4, 9 or 11). The CDRs of cetuximab include, V_{H} CDR 1 (amino acid residues to 31-35, according to Kabat definition, of SEQ ID NO: 2 or 7, set forth in SEQ ID NO: 35); V_{H} CDR 2 (amino acid residues 50-65 of SEQ ID NO: 2 or 7, set forth in SEQ ID NO: 36); V_{H} CDR 3 (amino acid residues 98-108 of SEQ ID NO: 2 or 7, set forth in SEQ ID NO: 37); V_{L} CDR 1 (amino acid residues 24-34 of SEQ ID NO: 4, 9 or 11, set forth in SEQ ID NO: 38); V_{L} CDR 2 (amino acid residues 50-56 of SEQ ID NO: 4, 9 or 11, set forth in SEQ ID NO: 39); and V_{L} CDR 3 (amino acid residues 89-97 of SEQ ID NO: 4, 9 or 11, set forth in SEQ ID NO: 40), see e.g., U.S. Publ. No. US20110117110.

Humanized versions of cetuximab have been generated in which the variable regions of the murine heavy and light chains have been humanized by amino acid replacements in the framework regions (see Table 5). For example, U.S. Pat. No. 7,060,808 describes H225, which contains a variable heavy chain having the sequence of amino acids set forth in SEQ ID NO: 14 and a variable light chain having the sequence of amino acids set forth in SEQ ID NO: 15. Another humanized variant, designated Hu225, is described in U.S. Published Appl. No. US 2011/0117110, which is an antibody that contains a variable heavy chain having the sequence of amino acids set forth in SEQ ID NO: 16 and a variable light chain having the sequence of amino acids set forth in SEQ ID NO: 17. The CDRs of the humanized variants are identical to M225 and to the C225 and other reported cetuximab antibodies as described above. These humanized antibodies exhibit reduced immunogenicity as compared to cetuximab. As described elsewhere herein with respect to the modified anti-EGFR variants provided herein, the humanized variants of cetuximab can be full-length antibodies or can be antigen-binding fragments thereof, including Fab′, F(ab′)₂, Fab, Fv, rIgG, and scFv fragments. As a full-length antibody, the humanized antibodies can possess any immunoglobulin isotype or class (e.g., IgG, IgM, IgD, IgE, IgA and IgY), any subclass (e.g., IgG1, IgG2, IgG3, IgG4, IgA1 and IgA2) or sub-subclass (e.g., IgG2a and IgG2b).

TABLE 5
Exemplary SEQ ID NOS of heavy chain (HC) and light
chain (LC) of Cetuximab or Cetuximab Derivatives

| | heavy chain | | light chain | |
|---|---|---|---|---|
| | (SEQ ID NO) | | (SEQ ID NO) | |
| | | variable region | | variable region |
| | full length | (1-119) | full length | (1-107) |
| | 1 | 2 | 3 | 4 |
| | 5 | 2 | 3 | 4 |
| | 12 | 2 | 13 | 4 |
| | 6 | 7 | 8 | 9 |
| | 6 | 7 | 10 | 11 |
| Humanized | — | 14 | — | 15 |
| | — | 16 | — | 17 |

Other cetuximab variants also have been described and are known in the art, which exhibit altered properties or activities (see, e.g., U.S. Pat. Nos. 7,657,380, 7,930,107, 7,060,808, 7,723,484, U.S. Pat. Publ. Nos. 2011014822, 2005142133, 2011117110, International Pat. Pub. Nos. WO2012003995, WO2010080463, WO2012020059, WO2008152537, and Lippow et al. (2007) Nat Biotechnol. 25(10):1171-1176).

The modifications described herein can be in any cetuximab, antigen-binding fragment or variant thereof, including any known in the art.

b. Function

Cetuximab specifically binds to EGFR. The crystal structure of cetuximab Fab bound to the extracellular domain of the EGFR (sEGFR) has been determined (Li et al., (2005) Cancer Cell 7:301-311). Cetuximab binds to domain III of the epidermal growth factor receptor (amino acids 310-514 of SEQ ID NO: 43), with an epitope that partially overlaps with the natural ligand epidermal growth factor. Residues ^{L27}Gln, ^{L50}Tyr ^{L94}Trp (e.g., with reference to the variable region set forth in SEQ ID NO: 4) and ^{H52}Trp, ^{H58}Asp, ^{H101}Tyr, ^{H102}Tyr, ^{H103}Asp and ^{H104}Tyr (e.g., with reference to the variable region set forth in SEQ ID NO: 2) of cetuximab make contacts with domain III of sEGFR. The light chain of cetuximab binds to the C-terminal domain of EGFR, with V_{L} CDR 1 residue ^{L27}Gln of cetuximab binding to residue N473 of sEGFR. V_{H} CDR 3 residue ^{H102}Tyr protrudes into a hydrophobic pocket on the surface of a large β sheet of domain III, making hydrogen bonds to glutamine side chains of Q384 and Q408 of sEGFR. V_{H} CDR 2 and V_{H} CDR 3 lie over the hydrophobic pocket, anchored by side chain to side chain hydrogen bonds between ¹¹⁵²Trp and 5418 of sEGFR and ^{H104}Tyr and 5468 of sEGFR, side chain to main chain interactions between ^{H54}Gly and ^{H103}Asp carbonyl oxygens and sEGFR 5440 and R353, and indirect hydrogen bonds between ^{H56}Asn and 5418 and Q384 of sEGFR. In addition to blocking the binding of EGF to sEGFR, the variable heavy chain of cetuximab sterically blocks domain I thereby preventing domain II from adopting a conformation necessary for dimerization.

Cetuximab binds to the extracellular domain of EGFR on both normal and tumor cells preventing ligand binding and subsequent activation (Li et al., (2005) Cancer Cell 7:301-311; Blick et al., (2007) Drugs 67(17):2585-2607). Cetuximab competitively inhibits the binding of epidermal growth factor and transforming growth factor alpha (TGF-alpha) preventing cell growth and metastatic spread. That is, binding of cetuximab blocks phosphorylation and activation of tyrosine-receptor kinases, resulting in inhibition of cell growth, induction of apoptosis, decreased matrix metalloprotease secretion and reduced vascular endothelial growth factor production. Cetuximab also can induce an antitumor effect through inhibition of angiogenesis. Cetuximab inhibits expression of VEGF, IL-8 and bFGF in the highly metastatic human TCC 253JB-V cells in a dose-dependent manner and decrease microvessel density (Perrotte et al. (1999), Clin. Cancer Res., 5:257-264). Cetuximab can down-regulate VEGF expression in tumor cells in vitro and in vivo (Petit et al. (1997), Am. J. Pathol., 151:1523-1530; Prewett et al. (1998), Clin. Cancer Res. 4:2957-2966). Cetuximab is also involved in complement activation and antibody-dependent cellular cytotoxicity (ADCC) and receptor internalization.

### C. MODIFIED ACTIVE ANTI-EGFR ANTIBODIES WITH ACIDIC pH SELECTIVITY

Provided herein are modified anti-EGFR antibodies or antigen-binding fragments that contain an amino acid replacement with glutamic acid (Glu, E) at a position corresponding to position 104 (designated 104E) of the variable domain of the heavy chain of an anti-EGFR antibody with reference to SEQ ID NO: 2 or 7. A position corresponding to position 104 in an unmodified anti-EGFR antibody can be determined by alignment of the variable heavy chain with the variable heavy chain set forth in SEQ ID NO: 2 or 7 (see, e.g., FIG. 2). Also provided herein are modified anti-EGFR antibodies or antigen-binding fragments that contain a corresponding replacement to the conservative amino acid aspartic acid (D) at a position corresponding to position 104 (designated 104E) of the variable domain of the heavy chain of an anti-EGFR antibody with reference to SEQ ID NO: 2 or 7.

The modified anti-EGFR antibodies provided herein that contain the amino acid replacement corresponding to 104E specifically bind to EGFR antigen (e.g., human EGFR) or soluble fragment thereof. The binding activity of the modified anti-EGFR antibodies provided herein is greater under conditions that include one or both of acidic pH of from 6.0 to 6.5, inclusive, and a lactate concentration of 15 mM to 20 mM, inclusive compared to under conditions that include one or both of neutral pH of or about 7.4 and a lactate concentration of or about 1 mM. For example, the ratio of binding activity under conditions that include one or both of pH 6.0 to 6.5 and 15 mM to 20 mM lactate versus binding activity under conditions that include one or both of or about pH 7.4 and/or of or about 1 mM lactate can be at least or greater than 1.1, 1.2, 1.3, 1.4, 1.5, 1.6, 1.7, 1.8, 1.9, 2.0, 2.5, 3.0, 3.5, 4.0, 4.5, 5.0, 6.0, 7.0, 8.0, 9.0, 10.0, 11.0, 12.0, 13.0, 14.0, 15.0, 20.0, 25.0, 30.0, 35.0, 40.0, 45.0, 50.0 or more. The modified anti-EGFR antibodies provided herein can exhibit the altered binding activity in the presence of physiologic concentrations of protein (e.g., 25% serum). Hence, the antibodies provided herein can exhibit tumor selective EGFR binding activity, whereby binding activity is greater under conditions that exist in a tumor microenvironment compared to conditions that exist in a non-tumor microenvironment.

The modified anti-EGFR antibody, or antigen-binding fragment thereof, provided herein minimally contain a variable heavy chain and a variable light chain, or a portion thereof that is sufficient to bind EGFR antigen (e.g., human EGFR), or a soluble fragment thereof, when assembled into an antibody, whereby at least the variable heavy chain is modified by replacement with 104E. The resulting modified anti-EGFR antibodies can be full-length IgG (e.g., IgG1) antibodies, or can be fragments thereof, for example, a Fab, Fab′, F(ab′)₂, single-chain Fv (scFv), Fv, dsFv, diabody, Fd and Fd′ fragments. Further, the resulting modified anti-EGFR antibodies can contain a domain other than IgG1.

The modified anti-EGFR antibody provided herein can contain only an amino acid replacement 104E, or a corresponding replacement to the conservative amino acid aspartic acid (D), in the variable heavy chain compared to the unmodified anti-EGFR antibody. In other examples of modified anti-EGFR antibodies provided herein, additional amino acid replacements or modifications in one or both of the heavy chain or light chain can be included in the anti-EGFR antibodies provided herein. For example, modified anti-EGFR antibodies provided herein can contain at least or 1, 2, 3, 4, 5, 6, 7, 8, 9, 10, 11, 12, 13, 14, 15, 16, 17, 18, 19, 20, or more modified positions compared to the anti-EGFR antibody not containing the modification. It is understood that in all examples of the modified anti-EGFR antibodies provided herein, the modified anti-EGFR antibody contains an amino acid replacement 104E, or corresponding conservative amino acid replacement, compared to the unmodified anti-EGFR antibody, and exhibits greater binding activity under conditions that include one or both of acidic pH of from 6.0 to 6.5, inclusive, and/or a lactate concentration of 15 mM to 20 mM, inclusive, compared to under conditions that include one or both of neutral pH of or about 7.4 and/or a lactate concentration of or of about 1 mM.

The unmodified anti-EGFR antibody can be a cetuximab antibody, antigen-binding fragment thereof or variant thereof. Exemplary unmodified anti-EGFR antibodies to which the amino acid replacement(s) herein can be made, including amino acid replacement 104E, include, but are not limited to, an anti-EGFR cetuximab antibody or antigen-binding fragment or variant thereof that contains a heavy chain set forth in any of SEQ ID NOS: 1, 2, 5, 6, 7, 12, 14 or 16, or an antigen-binding fragment or variant thereof containing at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity to any of SEQ ID NOS: 1, 2, 5, 6, 7, 12, 14 or 16. For example, an unmodified anti-EGFR antibody can contain a sequence of amino acids including a variable heavy chain (VH) set forth in SEQ ID NO: 2 and variable light chain (VL) set forth in SEQ ID NO: 4, a VH set forth in SEQ ID NO: 7 and a VL set forth in SEQ ID NO: 9, a VH set forth in SEQ ID NO: 7 and a VL set forth in SEQ ID NO: 11, a VH set forth in SEQ ID NO: 14 or a VL set forth in SEQ ID NO: 15, or a VH set forth in SEQ ID NO: 16 or a VL set forth in SEQ ID NO: 17, or variant thereof that contains a variable heavy and/or variable light chain that exhibits least 75%, 80%, 81%, 82%, 83%, 84%, 85%, 86%, 86%, 88%, 89%, 90%, 91%, 92%, 93%, 94%, 95%, 96%, 97%, 98%, 99% sequence identity to one or both of the variable heavy or light chains SEQ ID NOS. The unmodified anti-EGFR antibody can be a full-length antibody or antigen-binding fragment thereof. For example, the unmodified anti-EGFR antibody can contain any of the VH or VL regions above and a constant region of the heavy and light chain including a heavy chain set forth in SEQ ID NO: 1 and a light chain set forth in SEQ ID NO: 3, a heavy chain set forth in SEQ ID NO: 5 and a light chain set forth in SEQ ID NO: 3, a heavy chain set forth in SEQ ID NO: 12 and a light chain set forth in SEQ ID NO: 13, a heavy chain set forth in SEQ ID NO: 6 and a light chain set forth in SEQ ID NO: 8 or a heavy chain set forth in SEQ ID NO: 6 and a light chain set forth in SEQ ID NO: 10, or can be an antigen-binding fragment of the full-length antibody or variant thereof that contains a heavy and/or light chain that exhibits least 75%, 80%, 81%, 82%, 83%, 84%, 85%, 86%, 86%, 88%, 89%, 90%, 91%, 92%, 93%, 94%, 95%, 96%, 97%, 98%, 99% to one or both of the heavy or light chains SEQ ID NOS. In any of such examples, modified anti-EGFR antibodies or antigen-binding fragments thereof provided herein can contain a variable heavy chain with the amino acid replacement Y104E, where the tyrosine (Y) at a position corresponding to position 104 is replaced with E. In some examples, the amino acid residue that is modified (e.g., replaced) at the position corresponding to position 104 is a conservative residue or a semi-conservative amino acid residue to the amino acid set forth in SEQ ID NO: 2 or 7.

For purposes herein, reference to positions and amino acids for modification, including amino acid replacement or replacements, are with reference to the variable heavy chain of the wild-type cetuximab antibody, set forth in SEQ ID NO: 2 or 7, and the variable light of the wild-type cetuximab antibody chain, set forth in SEQ ID NO: 4. It is within the level of one of skill in the art to make any of the modifications in the variable heavy chain (including 104E) or variable light chain in another anti-EGFR antibody by identifying the corresponding amino acid residue in the variable heavy chain or variable light chain of the unmodified anti-EGFR antibody by alignment of the anti-EGFR antibody heavy chain or light chain with the reference anti-EGFR variable heavy chain set forth in SEQ ID NO: 2 or 7 or variable light chain set forth in SEQ ID NO: 4. For example, FIGS. 2A and 2B depict alignment of the heavy chain of exemplary anti-EGFR antibodies with SEQ ID NO: 2 and 7 and FIGS. 2C and 2D depict alignment of the light chain of exemplary anti-EGFR antibodies with SEQ ID NO: 4, 9 or 11. For purposes of modification (e.g., amino acid replacement), the corresponding amino acid residue at the replaced position can be any amino acid residue, and need not be identical to the residues set forth in SEQ ID NO: 2 or 7 or SEQ ID NO: 4. Typically, the corresponding amino acid residue identified by alignment with residues in SEQ ID NO: 2 or 7 or SEQ ID NO: 4 is an amino acid residue that is identical to SEQ ID NO: 2 or 7 or SEQ ID NO: 4, or is a conservative or semi-conservative amino acid residue thereto (see e.g., FIG. 2). As an example, the residue at the position corresponding to position 104 is a Tyr (Y) in SEQ ID NOS: 2 and 7. Thus, the corresponding residue in an unmodified anti-EGFR antibody that is replaced by glutamic acid (E), i.e., corresponding to Y104E in SEQ ID NO: 2 or 7, can be a conservative amino acid residue, such as tryptophan (Trp, W104E) or phenylalanine (Phe, F104E) (see Table 4).

It is also understood that the exemplary replacements provided herein can be made at the corresponding residue in an anti-EGFR antibody heavy chain or light chain, such as in the variable region of the heavy chain or light chain, as long as the replacement is different than the amino acid that exists in the unmodified form of the anti-EGFR antibody heavy chain or light chain. Based on this description and the description elsewhere herein, it is within the level of one of skill in the art to generate a modified anti-EGFR antibody containing any one or more of the described mutations, and test each for a property or activity as described herein.

The modified anti-EGFR antibodies provided herein can exhibit greater or increased binding activity to EGFR antigen (e.g., human EGFR or soluble form thereof) under conditions that include an acidic pH from 6.0 to 6.5, inclusive, and/or a weaker binding under conditions that include a neutral pH of 7.4 compared to the corresponding form of the unmodified anti-EGFR antibody, such as compared to the corresponding form of a wildtype cetuximab containing a heavy chain variable domain sequence of amino acids set forth in SEQ ID NO: 2 or 7. Typically, the modified anti-EGFR antibodies provided herein exhibit weaker binding activity to EGFR antigen or soluble fragment thereof (e.g., human EGFR or soluble form thereof) at neutral pH of 7.4 compared to the corresponding form of the unmodified anti-EGFR antibody, such as a compared to the corresponding form of a wildtype cetuximab containing a heavy chain variable domain sequence of amino acids set forth in SEQ ID NO: 2 or 7. In particular examples, the antibodies provided herein retain or exhibit similar or increased binding activity at pH 6.0 to pH 6.5, inclusive, compared to binding activity of the unmodified anti-EGFR antibody under the same conditions, but exhibit decreased binding activity at neutral pH of about pH 7.4, such as less than 20%, 30%, 40%, 50%, 60%, 70%, 80%, 90%, 95% binding activity at pH 7.4, than the corresponding form of the unmodified anti-EGFR antibody. For example, the modified anti-EGFR antibodies provided herein exhibit at least or about at least 2-fold, 3-fold, 4-fold, 5-fold, 6-fold, 7-fold, 8-fold, 9-fold, 10-fold, 11-fold, 12-fold, 13-fold, 14-fold, 15-fold, 20-fold, 25-fold, 30-fold, 40-fold, 50-fold or more weaker binding activity at neutral pH of 7.0 to 7.4, inclusive, compared to the corresponding form of the unmodified anti-EGFR antibody.

The binding activity to an EGFR antigen (e.g., human EGFR or soluble form thereof) can be determined or assessed based on any methods known to a person of skill in the art to assess binding of an antibody, or antigen-binding fragment, to EGFR (e.g., human EGFR). Examples of such assays are described in Section E. Such assays include, but are not limited to, solid phase-binding assay such as an immunoassay (e.g., enzyme-linked immunosorbent assay; ELISA) affinity-based biosensor assay (e.g., BIAcore technology), or in vivo binding assays. In such assays, the binding activity can be measured or represented as a detectable signal (e.g., spectrophotometric measurement or fluorescent measurement of binding), the concentration of half-maximal binding (EC₅₀) or a kinetic measure of binding (e.g., dissociation constant, K_{d}, association constant Kₐ, off-rate or other kinetic parameter of binding affinity). A skilled artisan understands that, depending on the particular assay used, a higher binding activity can be represented in some instances by a higher value and a weaker binding activity can be represented by a lower value (e.g., when binding activity is represented as the K_{A} or when represented as a measurement of binding signal). In other instances, a higher binding activity can be represented as a lower value and a weaker binding activity can be measured as a higher value (e.g., when binding activity is represented as the KD or off-rate).

For purposes herein, it is understood that a ratio of binding activity of at least or greater than 1.1, 1.2, 1.3, 1.4, 1.5, 1.6, 1.7, 1.8, 1.9, 2.0, 2.5, 3.0, 3.5, 4.0, 4.5, 5.0, 6.0, 7.0, 8.0, 9.0, 10.0, 11.0, 12.0, 13.0, 14.0, 15.0, 20.0, 25.0, 30.0, 35.0, 40.0, 45.0, 50.0 or more means that the modified anti-EGFR antibody exhibits the fold-difference higher binding activity (e.g., higher or tighter binding affinity) for EGFR antigen (e.g., human EGFR or a soluble fragment thereof) under conditions that include one or both of pH 6.0 to 6.5 and/or 15 mM to 20 mM lactate than under conditions that include pH 7.4 and 1 mM lactate. For example, a ratio of binding activity of at least 2.0 means that there is at least 2-fold tighter affinity, a ratio of binding activity of at least 3.0 means that there is at least 3-fold tighter affinity, a ratio of binding activity of at least 4.0 means that there is at least 4-fold tighter affinity, a ratio of binding activity of at least 5.0 means that there is at least 5-fold tighter affinity, a ratio of binding activity of at least 10.0 means that there is at least 10-fold tighter affinity.

In other examples, a ratio of binding activity of at least 2.0 means that the antibody exhibits an off-rate that is at least 2 times slower, a ratio of binding activity of at least 3.0 means that the antibody exhibits an off-rate that is at least 3 times slower, a ratio of binding activity of at least 4.0 means that the antibody exhibits an off-rate that is at least 4 times slower, a ratio of binding activity of at least 5.0 means that the antibody exhibits an off-rate that is at least 5 times slower, a ratio of binding activity of at least 10.0 means that the antibody exhibits an off-rate that is at least 10 times slower. In such examples, when binding activity is measured as an EC₅₀, K_{D}, a higher binding activity (e.g., tighter binding affinity) is represented by a lower concentration, such that a ratio of binding activity at pH 6.0 to 6.5 and/or 15 mM to 20 mM lactate versus pH 7.4, 1 mM lactate is represented as the quotient of the inverse of the EC₅₀ or K_{d} at pH 6.0 to 6.5 and/or 15 mM to 20 mM lactate versus the inverse of the EC₅₀ or K_{D} at pH 7.4, 1 mM lactate. As an example, the ratio of binding activity of an antibody that is measured to have an EC₅₀ of 4 mM at pH 6.0 to 6.5 and 15 mM to 20 mM lactate and an EC₅₀ of 16 mM at pH 7.4, 1 mM lactate is 4.0 (1/4/1/16).

The modified anti-EGFR antibodies, or antigen-binding fragments provided herein, typically have a dissociation constant (K_{D}) for binding EGFR (e.g., human EGFR) or a soluble fragment thereof that is less than 1×10⁻⁸M, 5×10⁻⁹ M, 1×10⁻⁹M, 5×10⁻¹° M, 1×10⁻¹⁰ M, 5×10⁻¹¹ M, 1×10⁻¹¹ M or less under conditions that include acidic pH 6.0 to 6.5, inclusive, and/or 15 mM to 20 mM lactate. The modified anti-EGFR antibodies, or antigen-binding fragments thereof provided herein, typically have an association constant (K_{A}) for binding EGFR (e.g., human EGFR) or a soluble fragment thereof that is greater than 1×10⁸ M⁻¹, 5×10⁹M⁻¹, 1×10⁹M⁻¹, 5×10¹⁰ M⁻¹, 1×10¹⁰ M⁻¹, 5×10¹¹M⁻¹, 1×10¹¹M⁻¹ or more under conditions that include acidic pH 6.0 to 6.5, inclusive and/or 15 mM to 20 mM lactate. In other examples, the modified anti-EGFR antibodies, or antigen-binding fragment thereof provided herein, typically have an EC₅₀ for binding EGFR (e.g., human EGFR), or a soluble fragment thereof, that is less than 10 mM, 5 mM, 4 mM, 3 mM, 2 mM, 1 mM or less under conditions that include acidic pH 6.0 to 6.5, inclusive, and/or 15 mM to 20 mM lactate. In particular examples, the anti-EGFR antibodies provided herein exhibit at least a 1.5-fold, 2-fold, 2.5-fold, 3-fold, 4-fold, 5-fold, 6-fold, 7-fold, 8-fold, 9-fold, 10-fold or more decrease in binding affinity (e.g., K_{d} or EC₅₀) for EGFR antigen (e.g., human EGFR or soluble fragment) at pH 7.4, 1 mM lactate while retaining comparable binding to EGFR at pH 6.0 to 6.5, inclusive, 16.6 mM lactate, and hence exhibit a greater ratio of binding activity (e.g., higher affinity or tighter affinity binding) at pH 6.0 to 6.5, inclusive, and/or 15 mM to 20 mM lactate compared to pH 7.4, 1 mM lactate of at least or greater than 1.1, 1.2, 1.3, 1.4, 1.5, 1.6, 1.7, 1.8, 1.9, 2.0, 2.5, 3.0, 3.5, 4.0, 4.5, 5.0, 6.0, 7.0, 8.0, 9.0, 10.0, 11.0, 12.0, 13.0, 14.0, 15.0, 20.0, 25.0, 30.0, 35.0, 40.0, 45.0, 50.0 or more.

Hence, by virtue of the altered binding activity of the modified anti-EGFR antibodies provided herein, the antibodies exhibit increased binding selectivity or activity for EGFR antigen in a tumor microenvironment than in a non-tumor microenvironment (e.g., basal layer of the skin). An altered pH microenvironment is the most common microenvironment found in tumor microenvironments (see e.g., Fogh Andersen et al. (1995) Clin. Chem., 41:1522-1525; Bhujwalla et al. (2002) NMR Biomed., 15:114-119; Helmlinger et al. (1997) Nature Med., 3:177; Gerweck and Seetharaman (1996), Cancer Res. 56(6):1194-1198). For example, in many tumors the ‘Warburg effect’ creates a microenvironment with a pH ranging from 5.6 to 6.8. Also, elevated lactate levels have been found associated with a variety of tumors including, but not limited to, head and neck, metastatic colorectal cancer, cervical cancer and squamous cell carcinoma (see e.g., Walenta et al. (1997) American Journal of Pathology 150(2): 409-415; Schwickert et al. (1995) Cancer Research 55: 4757-4759; Walenta et al. (2000) Cancer Research 60: 916-921; Guo et al. (2004) J Nucl Med 45: 1334-1339; Mathupala et al. (2007) J Bioenerg Biomembr 39: 73-77; Holroyde et al. (1979) Cancer Research 39: 4900-4904; Schurr and (2007) Neuroscience 147: 613-619; Quennet et al. (2006) Radiotherapy and Oncology 81: 130-135). In many tumors, the ‘Warburg effect’ creates a microenvironment with lactate concentrations between 10 to 20 mM. In contrast to the tumor microenvironment, the dermis, where many side effects that result from administration of anti-EGFR antibodies are localized, exhibits a neutral pH (e.g., pH 7.4) and normal lactate levels (e.g., 0.5 M to 2 mM).

Generally, the modified anti-EGFR antibodies provided herein exhibit the ratio of activity in the presence of physiological levels of protein. In an in vivo or physiological environment, the interstitial protein concentration (such as albumin) is anywhere from 20-50% of plasma. Serum contains about 60-80 g/L protein, and various tissues have been demonstrated to contain 12 mg/mL to 40 mg/mL interstitial protein (see, e.g., Aukland and Reed (1993) Physiological Reviews, 73:1-78). Hence, the modified anti-EGFR antibodies provided herein can exhibit the ratio of binding activity in the presence of 10 mg/mL to 50 mg/mL protein, such as at least at least 12 mg/mL to 40 mg/mL protein (e.g., at least 12 mg/mL, 15 mg/mL, 20 mg/mL, 25 mg/mL, 30 mg/mL, 35 mg/mL or 40 mg/mL protein), which, for example, can be provided in serum, such as human serum, or as a serum albumin, such as human serum albumin, or other protein that does not interact with the antibody or receptor or otherwise directly alter antibody-receptor interactions. For example, the modified anti-EGFR antibodies provided herein can exhibit the ratio of binding activity in the presence of 20% to 50% serum (vol/vol), such as 20% to 50% human serum, such as at least 20%, 25%, 30%, 35%, 40%, 45% or 50% serum (vol/vol).

Thus, by virtue of the greater ratio of binding activity under conditions that include one or both of an acidic pH from 6.0 to 6.5, inclusive, and/or 15 mM to 20 mM lactate compared to under conditions that include neutral pH of 7.4, and 1 mM lactate the modified anti-EGFR antibodies provided herein exhibit greater binding activity to an EGFR antigen (e.g., human EGFR) in a tumor microenvironment than a non-diseased or non-tumor microenvironment environment, such as those found in the skin or basal layer of the skin. Thus, the modified anti-EGFR antibodies provided herein exhibit selective activity against tumors, and reduced binding activity to cells in non-tumor microenvironments. Such selectivity achieved by their conditional binding activity minimizes the undesired activity on non-tumor cells, such as basal keratinocytes of the skin. Thus, the modified anti-EGFR antibodies, or antigen binding fragments thereof, provided herein confer reduced or fewer side effects when administered to subjects.

The modified anti-EGFR antibodies provided herein can exhibit increased inhibitory activity against EGFR in a tumor microenvironment compared to a non-diseased environment. Such inhibitory activities include, but are not limited to, inhibition of ligand-induced phosphorylation, dimerization and/or cell growth. As a result of such activities, antibodies provided herein exhibit tumor growth inhibition when administered in vivo to a subject having a tumor, such as a solid tumor. Tumor growth can be inhibited 30%, 40%, 50%, 60%, 70%, 80%, 90% or more compared to the growth of tumors in the absence of administered antibody. The functional activity of the modified anti-EGFR antibodies provided herein can be less than, similar to or greater than existing anti-EGFR therapies, such as therapies with cetuximab, when assessed in tumor models, provided the activity in non-diseased tissues is reduced. Reduced activity is demonstrated, for example, by decreased incidence or severity of a skin rash. For example, the provided anti-EGFR antibodies, or antigen binding fragments thereof, exhibit reduced dermal toxicity. Dermal toxicity, such as skin rash, can be assessed by standard assays known to one of skill in the art and described herein. For example, the anti-EGFR antibodies, or antigen binding fragments thereof, provided herein exhibit at least a 1.5-fold, 2-fold, 2.5-fold, 3-fold, 4-fold, 5-fold, or more decreased rash, such as assessed in a primate model.

The modified anti-EGFR antibodies provided herein can be produced by standard recombinant DNA techniques known to one of skill in the art. Any method known in the art to effect mutation of any one or more amino acids in a target protein can be employed.

Methods include standard site-directed or random mutagenesis of encoding nucleic acid molecules, or solid phase polypeptide synthesis methods. For example, nucleic acid molecules encoding a heavy chain or light chain of an anti-EGFR antibody can be subjected to mutagenesis, such as random mutagenesis of the encoding nucleic acid, error-prone PCR, site-directed mutagenesis, overlap PCR, gene shuffling, or other recombinant methods. The nucleic acid encoding the anti-EGFR antibodies can then be introduced into a host cell to be expressed heterologously. Hence, also provided herein are nucleic acid molecules encoding any of the modified anti-EGFR antibodies provided herein.

Non-limiting examples of modified anti-EGFR antibodies, as provided herein, are described below.

1. Modified Anti-EGFR Antibodies Containing Y104E

Provided herein are modified anti-EGFR antibodies containing an amino acid replacement glutamic acid (E) at position 104 (104E) of an unmodified anti-EGFR antibody with reference to positions set forth in SEQ ID NO: 2 or 7. Further modifications (e.g., amino acid replacement), such as any described elsewhere herein below, can be incorporated into the heavy chain and/or light chain of anti-EGFR antibodies and EGFR-binding fragments, in addition to the 104E amino acid replacement, as long as the resulting modified anti-EGFR antibody or antigen-binding fragment thereof exhibits greater binding activity under conditions that include acidic pH of from 6.0 to 6.5, inclusive, and/or a lactate concentration of 15 mM to 20 mM, inclusive, compared to under conditions that include neutral pH of or about 7.4, and/or 1 mM lactate concentration. The further modifications can be in the variable heavy chain and/or variable light chain of the antibody or antigen-binding fragment thereof. Further modifications also can be made to an anti-EGFR antibody that also contains other modifications, including modifications in the variable regions of the antibody and modifications in the constant regions of the antibody, for example, in the C_{H}1, hinge, C_{H}2, C_{H}3 or C_{L} regions.

Also, it is understood that a 104E anti-EGFR antibody or antigen-binding fragment thereof, including any containing one or more additional modifications in the heavy chain and/or light chain as described herein below, can be further modified by humanization, as long as the resulting modified anti-EGFR antibody or antigen-binding fragment thereof exhibits greater binding activity under conditions that include acidic pH of from 6.0 to 6.5, inclusive, and/or a lactate concentration of 15 mM to 20 mM, inclusive, compared to under conditions that include neutral pH of or about 7.4 and/or a lactate concentration of or about 1 mM.

The amino acid replacement(s), including amino acid replacement 104E, can be made in an unmodified anti-EGFR antibody containing: a variable heavy chain having a sequence of amino acids set forth in SEQ ID NO: 2 and a variable light chain having a sequence set forth in SEQ ID NO: 4, a variable heavy chain having the sequence of amino set forth in SEQ ID NO: 7 and a variable light chain having the sequence of amino acids set forth in SEQ ID NO: 9, or a variable heavy chain having the sequence of amino set forth in SEQ ID NO: 7 and a variable light chain having the sequence of amino acids set forth in SEQ ID NO: 11, or in an unmodified anti-EGFR antibody that contains a variant of the variable heavy chain set forth in SEQ ID NO: 2 or 7 and/or contains a variant of the light chain set forth in SEQ ID NO: 4, 9 or 11 that exhibit at least 65%, 70%, 75%, 80%, 85%, 86%, 87%, 88%, 89%, 90%, 91%, 92%, 93%, 94%, 95%, 96%, 97%, 98%, 99% or more sequence identity thereto. For example, the amino acid replacement can be made in a humanized cetuximab antibody containing: a variable heavy chain having the sequence of amino acids set forth in SEQ ID NO: 14 and a variable light chain having the sequence of amino acids set forth in SEQ ID NO: 15, or a variable heavy chain having the sequence of amino acids set forth in SEQ ID NO: 16 or a variable light chain having the sequence of amino acids set forth in SEQ ID NO: 17, or in sequence variants that exhibit at least 65%, 70%, 75%, 80%, 85%, 86%, 87%, 88%, 89%, 90%, 91%, 92%, 93%, 94%, 95%, 96%, 97%, 98%, 99% or more sequence identity to the variable heavy chain set forth in SEQ ID NO: 14 or 16 and/or the variable light chain set forth in SEQ ID NO: 15 or 17.

For example, provided herein are modified anti-EGFR antibodies containing an amino acid replacement 104E containing a heavy chain variable domain having the sequence of amino acids set forth in any of SEQ ID NOS: 74 or 75, or a sequence of amino acids that is at least 65%, 70%, 75%, 80%, 85%, 86%, 87%, 88%, 89%, 90%, 91%, 92%, 93%, 94%, 95%, 96%, 97%, 98%, 99% identical to any of SEQ ID NOS: 74 or 75 and contains at least the amino acid replacement 104E; and a light chain variable domain set forth in any of SEQ ID NOS: 4, 9, 11, 15 or 17, or a sequence of amino acids that is at least 65%, 70%, 75%, 80%, 85%, 86%, 87%, 88%, 89%, 90%, 91%, 92%, 93%, 94%, 95%, 96%, 97%, 98%, 99% identical to any of SEQ ID NOS: 4, 9, 11, 15 or 17. The modified anti-EGFR antibodies provided herein can be a full-length antibody or an antigen-binding fragment thereof that contains a sufficient portion of the variable heavy chain or variable light chain to bind antigen when assembled into an antibody, wherein the variable heavy chain at least contains the amino acid replacement 104E. Exemplary of such modified anti-EGFR antibodies are provided below.

a. Additional Modifications

Also provided herein are 104E anti-EGFR antibodies or antigen binding fragments thereof that can contain modifications in addition to the 104E amino acid replacement. The additional modifications can be single amino acid modifications, such as single amino acid replacements or substitutions, insertions or deletions, or multiple amino acid modifications, such as multiple amino acid replacements, insertions or deletions. Exemplary modifications are amino acid replacements, including single or multiple amino acid replacements. Modified anti-EGFR antibodies provided herein can contain at least 2, 3, 4, 5, 6, 7, 8, 9, 10, 11, 12, 13, 14, 15, 16, 17, 18, 19, 20, or more modified positions compared to the unmodified anti-EGFR antibody not containing the modification(s). The amino acid replacement(s) can be conservative substitution(s), such as set forth in Table 4, or a non-conservative substitution, such as any described herein.

### i. Additional Heavy Chain Modifications

Provided herein are modified anti-EGFR antibodies that contain an amino acid replacement of Glu (E) at position 104 (i.e., 104E), and optionally additional modification(s), such as one or more amino acid replacement(s), in a variable heavy chain of an unmodified anti-EGFR antibody (e.g., cetuximab), antigen-binding fragment thereof or variant thereof. The resulting modification(s) can be in a variable heavy chain set forth in SEQ ID NO: 2 or 7, or a variant thereof having at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity thereto. For example, the resulting modifications can be in an unmodified anti-EGFR antibody containing a variable heavy chain set forth in SEQ ID NO: 14 or SEQ ID NO: 16, or in a variant thereof or portion thereof having at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity thereto. The modifications also can be in a full-length heavy chain containing any of the above variable heavy chains, such as any set forth in any of SEQ ID NOS: 1, 5, 6, or 12, or in a variant thereof or portion thereof having at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity thereto. The modification can be in a complementarity determining region (CDR) or in a framework region.

For example, the modified anti-EGFR antibodies or antigen-binding fragments thereof can contain any one or more amino acid replacements set forth in Table 32. In particular, provided herein are modified anti-EGFR antibodies or antigen-binding fragments thereof containing a variable heavy chain, or portion thereof, with the amino acid replacement 104E and one or more other amino acid replacement(s) or substitution(s) at any of positions corresponding to positions 23, 24, 25, 26, 27, 28, 29, 30, 31, 32, 33, 34, 35, 36, 50, 51, 52, 53, 54, 55, 56, 57, 58, 59, 60, 61, 62, 63, 64, 65, 66, 67, 68, 69, 70, 71, 72, 73, 74, 75, 76, 77, 93, 94, 97, 98, 99, 100, 101, 102, 103, 105, 106, 107, 108, 109, 110, 111 or 112 with reference to the amino acid positions set forth in SEQ ID NO: 2 or 7. For example, the amino acid positions can be replacements at positions corresponding to replacement of Threonine (T) at position 23 (T23), V24, S25, G26, F27, S28, L29, T30, N31, Y32, G33, V34, H35, W36, V50, I51, W52, S53, G54, G55, N56, T57, D58, Y59, N60, T61, P62, F63, T64, S65, R66, L67, S68, I69, N70, K71, D72, N73, S74, K75, S76, Q77, Y93, Y94, R97, A98, L99, T100, Y101, Y102, D103, E105, F106, A107, Y108, W109, G110, Q111 or G112 with reference to the amino acid positions set forth in SEQ ID NO: 2. In some examples, the amino acid residue that is modified (e.g., replaced) at the position corresponding to any of the above positions is a conservative residue or a semi-conservative amino acid residue to the amino acid set forth in SEQ ID NO: 2 or 7 (see e.g., FIG. 2A or 2B).

The amino replacement at the position can be replacement to any other amino acid at the position, as long as the resulting modified anti-EGFR antibody or antigen-binding fragment thereof exhibit specific binding to EGFR antigen (e.g., human EGFR). Typically, the resulting anti-EGFR antibody, or antigen-binding fragment thereof, containing a further modification, exhibits greater binding activity under conditions that include acidic pH of from 6.0 to 6.5, inclusive, and/or a lactate concentration of 15 mM to 20 mM, inclusive, compared to under conditions that include neutral pH of or about 7.4 and/or a lactate concentration of or about 1 mM, such that the ratio of binding activity is greater than 1.0, such as greater than 1.2, 1.3, 1.4, 1.5, 1.6, 1.7, 1.8, 1.9, 2.0, 3.0, 4.0, 5.0, 6.0. 7.0, 8.0, 9.0, 10.0, 20.0, 40.0, 30.0, 40.0 or more as described herein above.

Provided herein are modified anti-EGFR antibodies or antigen-binding fragments thereof containing a variable heavy chain, or portion thereof, with the amino acid replacement 104E and one or more other amino acid replacement(s) corresponding to replacements set forth in Table 6 with reference to positions set forth in SEQ ID NO: 2 or 7.

TABLE 6
Exemplary additional heavy chain amino acid replacements

| | | | | | |
|---|---|---|---|---|---|
| T023K | T030H | G054D | S065P | N073R | T100S |
| T023H | T030R | G054P | S065Q | N073L | T100V |
| T023R | T030D | G054S | S065T | N073A | T100Y |
| T023A | T030G | G055H | S065W | N073C | Y101H |
| T023C | T030I | G055R | S065Y | N073G | Y101E |
| T023E | T030M | G055M | R066L | N073I | Y101F |
| T023G | T030N | G055S | R066A | N073M | Y101M |
| T023I | T030P | G055Y | R066C | N073P | Y101W |
| T023M | T030S | N056K | R066E | N073Q | Y102R |
| T023N | T030V | N056A | R066F | N073S | Y102C |
| T023P | T030W | N056P | R066N | N073T | Y102D |
| T023S | T030Y | N056S | R066P | N073V | Y102I |
| T023V | N031K | N056V | R066Q | N073W | Y102N |
| T023W | N031H | N056G | R066S | N073Y | Y102W |
| T023L | N031D | T057H | R066T | S074K | D103R |
| V024R | N031E | T057R | R066V | S074H | D103L |
| V024A | N031G | T057L | R066G | S074R | D103A |
| V024F | N031I | T057A | L067A | S074L | D103C |
| V024G | N031T | T057C | L067C | S074A | D103I |
| V024I | N031V | T057D | L067D | S074C | D103P |
| V024M | N031L | T057F | L067E | S074D | D103Q |
| V024P | Y032H | T057M | L067I | S074E | D103Y |
| V024S | Y032R | T057N | L067M | S074G | E105H |
| V024T | Y032C | T057Q | L067Q | S074I | E105T |
| V024L | Y032M | T057W | L067S | S074M | F106L |
| V024E | Y032N | T057Y | L067T | S074P | F106V |
| S025H | Y032T | D058L | L067V | S074T | F106W |
| S025R | Y032V | D058G | L067Y | S074V | F106Y |
| S025A | Y032L | D058M | L067G | S074Y | A107K |
| S025C | G033E | D058N | S068K | K075H | A107H |
| S025D | G033M | D058Q | S068H | K075R | A107R |
| S025E | G033S | Y059H | S068R | K075L | A107L |
| S025F | G033T | Y059R | S068L | K075A | A107C |
| S025G | G033Y | Y059A | S068C | K075C | A107D |
| S025I | V034A | Y059C | S068D | K075E | A107E |
| S025M | V034C | Y059D | S068E | K075F | A107G |
| S025P | V034I | Y059E | S068F | K075M | A107N |
| S025Q | V034M | Y059G | S068G | K075Q | A107S |
| S025T | V034P | Y059I | S068I | K075T | A107T |
| S025V | V034L | Y059P | S068N | K075V | A107Y |
| S025L | H035I | Y059Q | S068Q | K075W | Y108K |
| G026H | H035Q | Y059S | S068T | K075Y | Y108H |
| G026R | W036K | Y059T | S068V | K075G | Y108R |
| G026D | W036A | Y059V | I069A | K075P | Y108L |
| G026F | W036I | Y059W | I069C | S076H | Y108C |
| G026M | W036V | N060K | I069G | S076R | Y108F |
| G026N | W036Y | N060A | I069Y | S076L | Y108I |
| G026P | V050K | N060C | N070H | S076A | Y108N |
| G026Q | V050H | N060D | N070R | S076C | Y108S |
| G026S | V050A | N060F | N070L | S076D | Y108T |
| G026Y | V050D | N060G | N070D | S076E | Y108V |
| G026L | V050E | N060P | N070E | S076F | Y108W |
| F027H | V050G | N060Q | N070F | S076M | W109I |
| F027R | V050I | N060S | N070G | S076P | W109M |
| F027A | V050N | N060T | N070I | S076Q | W109Y |
| F027D | V050Q | N060Y | N070P | S076T | G110R |
| F027E | V050T | T061N | N070Q | S076Y | G110A |
| F027G | V050L | T061Q | N070S | S076I | G110M |
| F027M | I051K | P062G | N070T | S076V | G110P |
| F027P | I051H | F063H | N070V | Q077H | G110T |
| F027Q | I051A | F063R | N070Y | Q077R | Q111K |
| F027S | I051C | F063L | K071H | Q077L | Q111H |
| F027T | I051E | F063A | K071R | Q077A | Q111R |
| F027V | I051G | F063C | K071L | Q077E | Q111L |
| F027W | I051N | F063D | K071A | Q077G | Q111D |
| F027Y | I051Q | F063G | K071C | Q077I | Q111E |
| F027L | I051S | F063M | K071F | Q077M | Q111G |
| S028K | I051V | F063N | K071G | Q077N | Q111M |
| S028H | I051Y | F063Q | K071Q | Q077S | Q111P |
| S028R | I051L | F063S | K071S | Q077V | Q111S |
| S028A | W052I | F063V | K071T | Q077W | Q111T |
| S028D | W052N | F063P | K071V | Q077Y | Q111W |
| S028I | W052Y | T064R | K071W | Y093H | Q111Y |
| S028M | S053H | T064L | K071Y | Y093V | Q111V |
| S028P | S053R | T064C | D072K | Y093W | Q111I |
| S028Q | S053A | T064F | D072H | Y094R | G112A |
| S028V | S053C | T064G | D072R | Y094L | G112N |
| S028W | S053G | T064N | D072L | R097H | G112P |
| S028L | S053I | T064Q | D072A | R097W | G112S |
| S028C | S053M | T064V | D072G | A098P | G112T |
| L029K | S053P | S065H | D072I | L099N | G112Y |
| L029H | S053Q | S065R | D072M | L099W | |
| L029A | S053L | S065L | D072N | T100H | |
| L029D | S053T | S065C | D072Q | T100L | |
| L029G | S053V | S065E | D072S | T100A | |
| L029I | S053Y | S065F | D072V | T100D | |
| L029M | G054H | S065G | D072W | T100I | |
| L029N | G054R | S065I | D072Y | T100N | |
| L029S | G054A | S065M | D072P | T100P | |
| L029V | G054C | S065N | N073H | T100Q | |

For example, modified anti-EGFR antibodies or antigen-binding fragments thereof provided herein contain a variable heavy chain, or portion thereof, having the amino acid replacement 104E and one or more other amino acid replacement(s) at a position or positions corresponding to 24, 25, 27, 28, 29, 30, 31, 32, 50, 53, 54, 58, 59, 63, 64, 67, 68, 72, 73, 74, 75, 76, 77, 97, 100, 101, 107, 111 with reference to positions set forth in any of SEQ ID NO: 2 or 7. For example, the additional replacement(s) can be at positions corresponding to valine (V) at position 24 (V24), S25, F27, S28, L29, T30, N31, Y32, V50, S53, G54, D58, Y59, F63, T64, L67, S68, D72, N73, S74, K75, S76, Q77, R97, T100, Y101, A107, Q111 with reference to the amino acid positions set forth in SEQ ID NO: 2 or 7. For example, exemplary modified anti-EGFR antibodies provided herein contain one or more additional amino acid replacement(s) corresponding to heavy chain replacement(s) V24I, V24L, V24E, S25C, S25G, S25I, S25M, S25V, S25Q, S25T, S25L, S25H, S25R, S25A, S25D, F27R, S28C, L29H, T30F, N31H, N31I, N31T, N31V, Y32T, V50L, S53G, G54D, G54S, G54R, G54C, G54P, D58M, Y59E, F63R, F63C, F63G, F63M, F63V, F63P, F63S, T64N, T64V, L67G, S68F, S68Q, D72K, D72L, D72P, D72M, D72W, N73Q, S74H, S74R, S74D, S74G, S74Y, K75H, K75G, K75W, K75P, S76I, S76V, Q77R, Q77E, R97H, T100I, T100P, Y101W, Y105V, A107N, Q111I, Q111P, and/or Q111V.

In particular examples, exemplary additional modifications provided herein include modification of a heavy chain variable domain of an anti-EGFR antibody or antigen-binding fragment thereof at position(s) corresponding to positions 24, 25, 27, 30, 53, 72, 97 and 111, with reference to the amino acid positions set forth in SEQ ID NO: 2 or 7. For example, the additional amino acid positions can be replacements at positions corresponding to valine (V) at position 24 (V24), S25, F27, T30, S53, D72, R97 or Q111 with reference to the amino acid positions set forth in SEQ ID NO: 2 or 7. For example, in addition to the replacement 104E, additional amino acid replacements in modified anti-EGFR antibodies provided herein, include, but are not limited to, replacement of a heavy chain residue with: glutamic acid (E) at a position corresponding to 24; C at a position corresponding to 25; V at a position corresponding to position 25; R at a position corresponding to 27; F at a position corresponding to position 30; G at a position corresponding to position 53; L at a position corresponding to position 72; H at a position corresponding to 97; or P at a position corresponding to 111. For example, the modified anti-EGFR antibodies provided herein can contain one or more additional amino acid replacement(s), such as 1, 2, 3, 4, 5, 6, 7, 8 or 9 amino acid replacement(s), corresponding to heavy chain replacements of V24E, S25C, S25V, F27R, T30F, S53G, D72L, R97H or Q111P with reference to the sequence of amino acids set forth in SEQ ID NO: 2 or 7.

For any of the amino acid replacements in a variable heavy chain provided herein above, it is understood that the replacements can be made in the corresponding position in another anti-EGFR antibody by alignment therewith with the sequence set forth in SEQ ID NO: 2 or 7 (see, e.g., FIG. 2A or 2B), whereby the corresponding position is the aligned position. Hence, the antibody can contain a heavy chain constant region, or portion thereof. In particular examples, the amino acid replacement(s) can be at the corresponding position in a cetuximab heavy chain, or portion thereof, such as set forth in any of SEQ ID NOS: 1, 2, 5, 6, 7, 12, 14 or 16 or a variant thereof having at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity thereto. Generally, the modified anti-EGFR antibody exhibits greater binding activity under conditions that include acidic pH of from 6.0 to 6.5, inclusive, and/or a lactate concentration of 15 mM to 20 mM, inclusive, compared to under conditions that include neutral pH of or about 7.4 and/or a lactate concentration of or about 1 mM, such that the ratio of binding activity is greater than 1.0, such as greater than 1.2, 1.3, 1.4, 1.5, 1.6, 1.7, 1.8, 1.9, 2.0, 3.0, 4.0, 5.0, 6.0, 7.0, 8.0, 9.0, 10.0, 20.0, 30.0, 40.0, 50.0 or more.

Non-limiting amino acid replacements in the heavy chain variable domain of an unmodified anti-EGFR antibody or antigen-binding fragment thereof are set forth in Table 7. The Table sets forth exemplary heavy chain amino acid sequences designated by a SEQ ID NO. Examples of such modified anti-EGFR antibodies containing the modified heavy chain and a light chain are provided below. The modified anti-EGFR, or antigen-binding fragment thereof, can contain further additional modifications in the light chain, for example as described in the following subsection (C.1.a.ii), or as a result of humanization of the antibody as described herein, for example as described in subsection C.2.

TABLE 7
Exemplary Heavy Chain Amino Acid Replacements

| | | Heavy Chain |
|---|---|---|
| | Heavy Chain | Variable Domain |
| Amino Acid Replacements | (SEQ ID NO) | (SEQ ID NO) |
| HC-Y104E/HC-Q111P | 76 | 77, 78 |
| HC-S25C/HC-Y104E | 79 | 80, 81 |
| HC-S53G/HC-Y104E | 82 | 83, 84 |
| HC-S53G/HC-Y104E/HC-Q111P | 85 | 86, 87 |
| HC-S25V/HC-Y104E | 88 | 89, 90 |
| HC-S25V/HC-Y104E/HC-Q111P | 91 | 92, 93 |
| HC-S25V/HC-S53G/HC-Y104E | 94 | 95, 96 |
| HC-S25V/HC-S53G/HC-Y104E/ | 97 | 98, 99 |
| HC-Q111P | | |
| HC-T30F/HC-Y104E | 100 | 101, 102 |
| HC-T30F/HC-Y104E/HC-Q111P | 103 | 104, 105 |
| HC-T30F/HC-S53G/HC-Y104E | 106 | 107, 108 |
| HC-T30F/HC-S53G/HC-Y104E/ | 109 | 110, 111 |
| HC-Q111P | | |
| HC-D72L/HC-Y104E | 112 | 113, 114 |
| HC-D72L/HC-Y104E/HC-Q111P | 115 | 116, 117 |
| HC-S53G/HC-D72L/HC-Y104E | 118 | 119, 120 |
| HC-S53G/HC-D72L/HC-Y104E/ | 121 | 122, 123 |
| HC-Q111P | | |
| HC- F027G/Y104E | 315 | 316, 317 |
| HC- F027G/Y104E/Q111P | 318 | 319, 320 |
| HC- F027G/S053G/Y104E | 321 | 322, 323 |
| HC- F027G/S053G/Y104E/Q111P | 324 | 325, 326 |

### ii. Additional Light Chain Modifications

Provided herein are modified anti-EGFR antibodies that contain an amino acid replacement of Glu (E) at position 104 (i.e., 104E), and optionally additional modification(s), such as one or more amino acid replacement(s), in a variable light chain of an unmodified anti-EGFR antibody (e.g., cetuximab), antigen-binding fragment thereof, or variant thereof. The resulting modification(s) can be in a variable light chain set forth in any of SEQ ID NOS: 4, 9 or 11, or in a variant thereof, having at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity thereto. For example, the resulting modifications can be in an unmodified anti-EGFR antibody containing a variable light chain set forth in SEQ ID NO: 15 or SEQ ID NO: 17, or in a variant thereof, having at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity thereto. The modifications also can be in a full-length light chain containing any of the above variable light chains, such as set forth in any of SEQ ID NOS: 3, 8, 10 or 13, or in a variant thereof, having at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity thereto. The modification(s) can be in a complementarity determining region (CDR) or in a framework region.

For example, provided herein are modified anti-EGFR antibodies or antigen-binding fragments thereof a variable heavy chain with the amino acid replacement 104E, and containing at least one amino acid replacement or substitution in the variable light chain, or a portion thereof, at any of positions corresponding to 1, 2, 3, 4, 5, 24, 25, 26, 27, 28, 29, 30, 31, 32, 33, 48, 49, 51, 52, 53, 54, 55, 56, 86, 87, 89, 91, 92, 93, 96, 97, 98, 99 or 100 with reference to the amino acid positions set forth in SEQ ID NO: 4. For example, the amino acid positions can be replacements at positions corresponding to replacement of aspartic acid (D) at position 1 (D1), I2, L3, L4, T5, R24, A25, S26, Q27, S28, I29, G30, T31, N32, 133, 148, K49, A51, S52, E53, S54, I55, S56, Y86, Y87, Q89, N91, N92, N93, T96, T97, F98, G99 or A100 with reference to the amino acid positions set forth in SEQ ID NO: 4. In some examples, the amino acid residue that is modified (e.g., replaced) at the position corresponding to any of the above positions is a conservative residue or a semi-conservative amino acid residue to the amino acid set forth in any of SEQ ID NOS: 4.

The amino replacement at the position can be replacement to any other amino acid at the position, as long as the resulting modified anti-EGFR antibody or antigen-binding fragment thereof exhibit specific binding to EGFR antigen (e.g., human EGFR). Typically, the resulting anti-EGFR antibody, or antigen-binding fragment thereof, containing a further modification, exhibits greater binding activity under conditions that include acidic pH of from 6.0 to 6.5, inclusive, and/or a lactate concentration of 15 mM to 20 mM, inclusive, compared to under conditions that include neutral pH of or about pH 7.4, and/or a lactate concentration of or about 1 mM, such that the ratio of binding activity is greater than 1.0, such as greater than 1.2, 1.3, 1.4, 1.5, 1.6, 1.7, 1.8, 1.9, 2.0, 3.0, 4.0, 5.0, 6.0. 7.0, 8.0, 9.0, 10.0, 20.0, 40.0, 30.0, 40.0 or more as described herein above.

Provided herein are modified anti-EGFR antibodies or antigen-binding fragments thereof containing: a variable heavy chain, or portion thereof, with the amino acid replacement 104E; and a variable light chain containing one or more other amino acid replacement(s) in the variable light chain corresponding to replacements set forth in Table 8 with reference to positions set forth in SEQ ID NO: 4.

TABLE 8
Exemplary light chain amino acid replacements

| | | | | | |
|---|---|---|---|---|---|
| D001W | R024M | G030A | K049V | Y087D | T097D |
| I002C | R024S | G030E | K049Y | Y087F | T097G |
| I002V | R024W | G030F | K049L | Y087G | T097Q |
| I002W | R024Y | G030I | K049H | Y087I | T097S |
| L003D | R024G | G030M | K049R | Y087N | T097V |
| L003F | A025C | G030P | A051T | Y087P | T097K |
| L003G | A025G | G030Q | A051L | Y087S | T097R |
| L003S | A025L | G030S | S052A | Y087T | F098A |
| L003T | A025V | G030V | S052C | Y087V | F098M |
| L003V | S026A | G030Y | S052D | Y087W | F098S |
| L003W | S026C | G030L | S052E | Y087K | F098V |
| L003Y | S026D | G030K | S052G | Y087H | F098Y |
| L003R | S026I | G030H | S052I | Y087R | G099L |
| L004C | S026M | G030R | S052M | Q089E | G099D |
| L004E | S026N | T031A | S052Q | N091L | G099E |
| L004F | S026V | T031F | S052V | N091A | G099F |
| L004I | S026W | T031G | S052W | N091C | G099I |
| L004P | S026L | T031M | S052R | N091I | G099M |
| L004S | S026G | T031S | S052K | N091M | G099N |
| L004T | S026H | T031V | E053G | N091S | G099S |
| L004V | S026R | T031W | S054M | N091T | G099T |
| L004W | Q027A | T031L | I055A | N091V | G099V |
| L004K | Q027D | T031K | I055F | N091H | G099K |
| L004H | Q027E | T031H | S056G | N091R | G099H |
| L004R | Q027F | N032G | S056L | N092C | Q100C |
| T005A | Q027I | I033F | S056A | N092D | Q100D |
| T005C | Q027M | I033G | S056C | N092L | Q100E |
| T005D | Q027N | I033M | S056D | N092M | Q100F |
| T005E | Q027P | I033T | S056E | N092S | Q100I |
| T005F | Q027T | I033V | S056F | N092T | Q100M |
| T005G | S028A | I033H | S056N | N092V | Q100N |
| T005N | S028D | I048M | S056P | N092W | Q100P |
| T005S | S028N | I048S | S056Q | N092Y | Q100T |
| T005W | S028Q | I048L | S056V | N092H | Q100V |
| T005L | S028L | I048K | S056W | N092K | Q100W |
| T005K | S028K | K049A | S056H | N092R | Q100Y |
| T005H | S028H | K049E | S056R | N093T | Q100K |
| T005R | I029A | K049F | S056K | T096L | Q100H |
| T005P | I029E | K049G | Y086F | T096C | Q100R |
| R024A | I029F | K049N | Y086M | T096M | |
| R024C | I029S | K049Q | Y086H | T096V | |
| R024F | I029T | K049S | Y087L | T097L | |
| R024L | I029R | K049T | Y087C | T097A | |

For example, exemplary modified anti-EGFR antibodies or antigen-binding fragments thereof provided herein contain: a variable heavy chain or portion thereof having the amino acid replacement 104E; and a variable light chain or portion thereof having one or more amino acid replacements at a position or positions corresponding to 4, 5, 24, 29, 56 or 91 with reference to positions set forth in any of SEQ ID NO: 4. For example, the amino acid positions can be a replacement(s) at positions corresponding to replacement of leucine (L) at position 4 (L4), T5, R24, I29, S56 or N91 with reference to the amino acid positions set forth in SEQ ID NO: 4. For example, exemplary modified anti-EGFR antibodies provided herein contain one or more amino acid replacements, such as at least 1, 2, 3, 4, 5 or 6 amino acid replacement(s) corresponding to light chain replacement or replacements L4C, L4F, L4V, T5P, R24G, I29S, S56H or N91V. For example, the anti-EGFR antibodies provided herein contain an amino acid replacement corresponding to a light chain replacement of I29S in a sequence of amino acids set forth in SEQ ID NO: 4.

For any of the amino acid replacements in a variable light chain provided herein above, it is understood that the replacements can be made in the corresponding position in another anti-EGFR antibody by alignment therewith with the sequence set forth in SEQ ID NO: 4, whereby the corresponding position is the aligned position. In particular examples, the amino acid replacement(s) can be at the corresponding position in a cetuximab heavy chain, or portion thereof, such as set forth in in any of SEQ ID NOS: 3, 4, 8-11, 13, 15, or 17, or in a variant thereof, having at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity thereto. Generally, the modified anti-EGFR antibody exhibits greater binding activity under conditions that include acidic pH of from 6.0 to 6.5, inclusive, and/or a lactate concentration of 15 mM to 20 mM, inclusive, compared to under conditions that include neutral pH of or about pH 7.4 and/or a lactate concentration of or about 1 mM, such that the ratio of binding activity is greater than 1.0, such as greater than 1.2, 1.3, 1.4, 1.5, 1.6, 1.7, 1.8, 1.9, 2.0, 3.0, 4.0, 5.0, 6.0, 7.0, 8.0, 9.0, 10.0, 20.0, 30.0, 40.0, 50.0 or more.

Non-limiting amino acid replacements in the variable light chain, in addition to the replacement 104E in the variable heavy chain, of an unmodified anti-EGFR antibody or antigen-binding fragment thereof are set forth in Table 9. The Table sets forth exemplary heavy chain amino acid sequences and light chain amino acid sequences designated by a SEQ ID NO. Examples of such modified anti-EGFR antibodies containing a modified heavy chain and modified light chain are provided below. The modified anti-EGFR, or antigen-binding fragment thereof, can contain further additional modifications in the heavy chain, for example, as described in the subsection above, or as a result of humanization of the antibody as described herein, for example as described in subsection C.2. Further, any of the modification(s) in a heavy chain as described above and any of the modification(s) in a light chain as described herein can be combined in an anti-EGFR antibody, or EGFR-binding fragment thereof.

TABLE 9
Exemplary Heavy Chain and Light Chain
Combined Amino Acid Replacements

| | Heavy Chain | Light Chain |
|---|---|---|
| Amino Acid Replacements | (SEQ ID NO) | (SEQ ID NO) |
| HC-Y104E/LC-I29S | 72, 74, 75 | 124, 125, 126, 127 |
| HC-Y104E/HC-Q111P/LC-I29S | 76, 77, 78 | 124, 125, 126, 127 |

### iii. Other Modifications

Any of the modified anti-EGFR antibodies provided herein also can contain one or more other additional modifications in the variable region or constant region of the heavy or light chain. Examples of other additional modifications that can be included in the modified anti-EGFR antibodies provided herein include, but are not limited to, those described in U.S. Pat. Nos. 7,657,380, 7,930,107, 7,060,808, 7,723,484, U.S. Pat. Publ. Nos. 20110142822, 2005142133, 2011117110, International Pat. Pub. Nos. WO2012003995, WO2010080463, WO2012020059, WO2008152537, and Lippow et al. (2007) Nat Biotechnol. 25(10):1171-1176. Non-limiting examples of exemplary amino acid modifications described in the art that can be included in any anti-EGFR antibody, or antigen binding fragment thereof, provided herein include:

variants containing an amino acid replacement (substitution) in the variable light chain (V_{L}) at one or more positions corresponding to replacement of aspartate at position 1 with glutamate (D1E), D1C, I2T, I2C, L3V, L3T, L3C, L4C, T5C, Q6C, S7C, P8C, V9C, V9A, V9D, V9G, V9P, V9S, I10T, I10S, I10F, I10C, L11Q, L11C, S12A, S12C, V13L, V13M, V135, V13A, V13C, S14T, S14C, P15V, P15L, P15C, G16K, G16C, E17D, E17K, E17C, R18V, R18K, R18C, V19A, V19T, V19C, S20T, S20C, S20A, F21I, F21L, F21C, S22T, S22C, R24P, A25V, A25S, A25I, A25P, A25T, A25Y, A25C, A25F, A25M, A25L, A25W, S26D, Q27W, Q27E, Q27F, Q27Y, Q27T, Q27H, S28R, S28F, G30Y, G30C, G30H, G30K, G30Q, G30R, G30W, G30F, G30T, G30M, G305, G30A, T31E, T31V, T31D, T31R, N32H, I33L, H34C, Q38K, R39K, T40P, T40S, N41G, N41D, G42Q, G42K, G42E, S43A, S43P, R45K, K49Y, K49F, Y50G, S53V, S60D, S60A, G64S, G64A, D70E, D70V, F71Y, S74T, N76S, N76T, S77R, S77G, V78L, E79Q, S80P, S80A, E81A, I83F, I183S, I83V, I83A, D85V, D85T, D85I, D85M, Y87S, Q89C, Q89H, Q90C, N91C, N91Q, N91L, N92C, N92L, N92R N92K, N92M, N92Y, N92H, N92E, N92F, N93A, N93D, N93E, N93V, N93K, N93C, W94F, W94Y, P95C, T96C, T96L, T96E, T97C, T97A, T97D, T97E, T97P, T97K, T97N, T97Q, T97I, T97G, T97L, T97H, T97R, T97S, G99A, A100G, A100Q, K103T, L104V and L1061, in the sequence of amino acids set forth in SEQ ID NO: 4;

variants containing an amino acid replacement (substitution) in the variable heavy chain (V_{H}) at positions corresponding to replacement of glutamine at position 1 with glutamic acid (Q1E), Q1C, V2C, Q3T, Q3C, L4C, K5Q, K5V, K5L, K5C, Q6E, Q6C, S7C, G8C, P9A, P9G, P9C, G10V, G10C, L11C, V12C, Q13K, Q13R, Q13C, P14C, S15G, S15T, S15C, Q16G, Q16R, Q16E, Q16C, S17T, S17C, L18C, S19K, S19R, S19T, S19C, I20L, I20C, T21S, T21C, T23A, T23K, T23C, V24A, V24C, S25C, F27G, S28N, S28T, L29I, T30S, T30K, N31V, N31D, N31I, N31T, N32S, Y32R, Y32W, G33A, G33D, G33E, G33Y, V34L, V34N, V34E, V34Q, V34S, V34W, H35S, V37I, S40A, S40P, P41T, G44A, L48V, L48I, G49S, G49A, V50L, V50Q, V50E, V50I, V50Y, V50N, I51G, I51M, I51S, I51Q, I51A, I51C, I51V, W52F, W52Y, W52G, W52T, S53Q, S53T, S53N, S53Y, G54A, G54V, G54L, G54I, G54S, G55D, G55A, G55E, G55H, G55F, N56A, N56G, N56S, N56T, T57A, T57D, T57G, T57S, T57E, T57P, D58Y, D58N, Y59A, Y59C, Y59E, Y59F, Y59G, Y59S, Y59W, T59H, Y59P, Y59Q, N60D, N60A, T61E, T61P, P62S, F63L, F63V, T64K, T64E, T64A, T64N, T64D, S65G, L67F, L67V, S68T, N70S, N70T, K71V, D72E, N73T, S74A, S76N, Q77T, Q77S, V78L, V78F, V78A, F79Y, F79S, F79V, F80L, F80M, K81Q, K81T, K81E, K81Q, M82L, N83T, N83S, S84N, L85M, L85V, Q86R, Q86D, Q86T, S87A, S87P, N88E, N88V, N88G, N88A, N88D, I92T, I92V, A96C, R97C, A98C, L99C, L99E, T100D, T100C, T100A, Y101C, Y101W, Y101A, Y102C, Y102F, Y102A, Y102W, D103E, D103P, D103C, E105C, E105N, E105D, E105Y, F106C, F106D, F106Y, A107C, A107D, Y108C and Y108F, in the sequence of amino acids set forth in SEQ ID NO: 2 or 7; and

variants containing amino acid replacement (substitution) in the heavy chain constant regions, for example, in the hinge, C_{H}2 and C_{H}3 regions, including replacement of proline at position 230 with alanine (P230A), E233D, L234D, L234E, L234N, L234Q, L234T, L234H, L234Y, L234I, L234V, L234F, L235D, L235S, L235N, L235Q, L235T, L235H, L235Y, L235I, L235V, L235F, S239D, S239E, S239N, S239Q, S239F, S239T, S239H, S239Y, V240I, V240A, V240T, V240M, F241W, F241L, F241Y, F241E, F241R, F243W, F243L F243Y, F243R, F243Q, P244H, P245A, P247V, P247G, V262I, V262A, V262T, V262E, V263I, V263A, V263T, V263M, V264L, V264I, V264W, V264T, V264R, V264F, V264M, V264Y, V264E, D265G, D265N, D265Q, D265Y, D265F, D265V, D265I, D265L, D265H, D265T, V266I, V266A, V266T, V266M, S267Q, S267L, S267T, S267H, S267D, S267N, E269H, E269Y, E269F, E269R, E269T, E269L, E269N, D270Q, D270T, D270H, E272S, E272K, E272I, E272Y, V273I, K274T, K274E, K274R, K274L, K274Y, F275W, N276S, N276E, N276R, N276L, N276Y, Y278T, Y278E, Y278K, Y278W, E283R, Y296E, Y296Q, Y296D, Y296N, Y296S, Y296T, Y296L, Y296I, Y296H, N297S, N297D, N297E, S298H, T299I, T299L, T299A, T299S, T299V, T299H, T299F, T299E, V302I, W313F, E318R, K320T, K320D, K320I, K322T, K322H, V323I, S324T, S324D, S324R, S324I, S324V, S324L, S324Y, N325Q, N325L, N325I, N325D, N325E, N325A, N325T, N325V, N325H, K326L, K326I, K326T, A327N, A327L, A327D, A327T, L328M, L328D, L328E, L328N, L328Q, L328F, L328I, L328V, L328T, L328H, L328A, P329F, A330L, A330Y, A330V, A330I, A330F, A330R, A330H, A330S, A330W, A330M, P331V, P331H, I332D, I332E, I332N, I332Q, I332T, I332H, I332Y, I332A, E333T, E333H, E333I, E333Y, K334I, K334T, K334F, T335D, T335R, T335Y, D221K, D221Y, K222E, K222Y, T223E, T223K, H224E, H224Y, T225E, T225E, T225K, T225W, P227E, P227K, P227Y, P227G, P228E, P228K, P228Y, P228G, P230E, P230Y, P230G, A231E, A231K, A231Y, A231P, A231G, P232E, P232K, P232Y, P232G, E233N, E233Q, E233K, E233R, E233S, E233T, E233H, E233A, E233V, E233L, E233I, E233F, E233M, E233Y, E233W, E233G, L234K, L234R, L234S, L234A, L234M, L234W, L234P, L234G, L235E, L235K, L235R, L235A, L235M, L235W, L235P, L235G, G236D, G236E, G236N, G236Q, G236K, G236R, G236S, G236T, G236H, G236A, G236V, G236L, G236I, G236F, G236M, G236Y, G236W, G236P, G237D, G237E, G237N, G237Q, G237K, G237R, G237S, G237T, G237H, G237V, G237L, G237I, G237F, G237M, G237Y, G237W, G237P, P238D, P238E, P238N, P238Q, P238K, P238R, P238S, P238T, P238H, P238V, P238L, P238I, P238F, P238M, P238Y, P238W, P238G, S239Q, S239K, S239R, S239V, S239L, S239I, S239M, S239W, S239P, S239G, F241D, F241E, F241Y, F243E, K246D, K246E, K246H, K246Y, D249Q, D249H, D249Y, R255E, R255Y, E258S, E258H, E258Y, T260D, T260E, T260H, T260Y, V262E, V262F, V264D, V264E, V264N, V264Q, V264K, V264R, V264S, V264H, V264W, V264P, V264G, D265Q, D265K, D265R, D265S, D265T, D265H, D265V, D265L, D265I, D265F, D265M, D265Y, D265W, D265P, S267E, S267Q, S267K, S267R, S267V, S267L, S267I, S267F, S267M, S267Y, S267W, S267P, H268D, H268E, H268Q, H268K, H268R, H268T, H268V, H268L, H268I, H268F, H268M, H268W, H268P, H268G, E269K, E269S, E269V, E269I, E269M, E269W, E269P, E269G, D270R, D270S, D270L, D270I, D270F, D270M, D270Y, D270W, D270P, D270G, P271D, P271E, P271N, P271Q, P271K, P271R, P271S, P271T, P271H, P271A, P271V, P271L, P271I, P271F, P271M, P271Y, P271W, P271G, E272D, E272R, E272T, E272H, E272V, E272L, E272F, E272M, E272W, E272P, E272G, K274D, K274N, K274S, K274H, K274V, K274I, K274F, K274M, K274W, K274P, K274G, F275L, N276D, N276T, N276H, N276V, N276I, N276F, N276M, N276W, N276P, N276G, Y278D, Y278N, Y278Q, Y278R, Y278S, Y278H, Y278V, Y278L, Y278I, Y278M, Y278P, Y278G, D280K, D280L, D280W, D280P, D280G, G281D, G281K, G281Y, G281P, V282E, V282K, V282Y, V282P, V282G, E283K, E283H, E283L, E283Y, E283P, E283G, V284E, V284N, V284T, V284L, V284Y, H285D, H285E, H285Q, H285K, H285Y, H285W, N286E, N286Y, N286P, N286G, K288D, K288E, K288Y, K290D, K290N, K290H, K290L, K290W, P291D, P291E, P291Q, P291T, P291H, P291I, P291G, R292D, R292E, R292T, R292Y, E293N, E293R, E293S, E293T, E293H, E293V, E293L, E293I, E293F, E293M, E293Y, E293W, E293P, E293G, E294K, E294R, E294S, E294T, E294H, E294V, E294L, E294I, E294F, E294M, E294Y, E294W, E294P, E294G, Q295D, Q295E, Q295N, Q295R, Q295S, Q295T, Q295H, Q295V, Q295I, Q295F, Q295M, Q295Y, Q295W, Q295P, Q295G, Y296K, Y296R, Y296A, Y296V, Y296M, Y296G, N297Q, N297K, N297R, N297T, N297H, N297V, N297L, N2971, N297F, N297M, N297Y, N297W, N297P, N297G, S298D, S298E, S298Q, S298K, S298R, S2981, S298F, S298M, S298Y, S298W, T299D, T299E, T299N, T299Q, T299K, T299R, T299L, T299F, T299M, T299Y, T299W, T299P, T299G, Y300D, Y300E, Y300N, Y300Q, Y300K, Y300R, Y300S, Y300T, Y300H, Y300A, Y300V, Y300M, Y300W, Y300P, Y300G, R301D, R301E, R301H, R301Y, V303D, V303E, V303Y, S304D, S304N, S304T, S304H, S304L, V305E, V305T, V305Y, K317E, K317Q, E318Q, E318H, E318L, E318Y, K320N, K320S, K320H, K320V, K320L, K320F, K320Y, K320W, K320P, K320G, K322D, K322S, K322V, K322I, K322F, K322Y, K322W, K322P, K322G, S324H, S324F, S324M, S324W, S324P, S324G, N325K, N325R, N325S, N325F, N325M, N325Y, N325W, N325P, N325G, K326P, A327E, A327K, A327R, A327H, A327V, A327I, A327F, A327M, A327Y, A327W, A327P, L328D, L328Q, L328K, L328R, L328S, L328T, L328V, L328I, L328Y, L328W, L328P, L328G, P329D, P329E, P329N, P329Q, P329K, P329R, P329S, P329T, P329H, P329V, P329L, P329I, P329M, P329Y, P329W, P329G, A330E, A330N, A330T, A330P, A330G, P331D, P331Q, P331R, P331T, P331L, P331I, P331F, P331M, P331Y, P331W, I332K, I332R, I332S, I332V, I332F, I332M, I332W, I332P, I332G, E333L, E333F, E333M, E333P, K334P, T335N, T335S, T335H, T335V, T335L, T335I, T335F, T335M, T335W, T335P, T335G, 1336E, I336K, I336Y, S337E, S337N, S337H, S298A, K326A, K326S, K326N, K326Q, K326D, K325E, K326W, K326Y, E333A, E333S, K334A, K334E, Y300I, Y300L, Q295K, E294N, S298N, S298V, S298D, D280H, K290S, D280Q, D280Y, K290G, K290T, K290Y, T250Q, T250E, M428L, M428F, S239D, S239E, S239N, S239Q, S239T, V240I, V240M, V264I, V264T, V264Y, E272Y, K274E, Y278T, N297D, T299A, T299V, T299I, T299H, K326T, L328A, L328H, A330Y, A330L, A330I, I332D, 1332E, I332N, and I332Q, according to EU index numbering.

b. Exemplary 104E Modified Anti-EGFR Antibodies and Fragments Thereof

Exemplary modified anti-EGFR antibodies provided herein that contain a replacement of the amino acid corresponding to position 104 with glutamic acid (i.e., 104E) with reference to the heavy chain variable domain set forth in SEQ ID NO: 2 or 7, and optionally one or more further amino acid replacement(s) in the heavy chain or light chain of the antibody, are described below. The modified anti-EGFR antibodies provided herein, such as any described herein, minimally contain a modified variable heavy chain and/or modified variable light chain, or portion thereof sufficient to specifically bind EGFR antigen (e.g., human EGFR) when assembled into an antibody. The 104E-containing anti-EGFR antibodies can exhibit greater binding activity under conditions that include acidic pH of from 6.0 to 6.5, inclusive, and/or a lactate concentration of 15 mM to 20 mM, inclusive, compared to under conditions that include neutral pH of or about 7.4 and/or a lactate concentration of or about 1 mM, such that the ratio of binding activity is greater than 1.0, such as greater than 1.2, 1.3, 1.4, 1.5, 1.6, 1.7, 1.8, 1.9, 2.0, 3.0, 4.0, 5.0, 6.0, 7.0, 8.0, 9.0, 10.0, 20.0, 30.0, 40.0, 50.0 or more.

For example, provided herein are 104E modified anti-EGFR antibodies containing: i) a modified variable heavy chain set forth in any of SEQ ID NOS: 74, 75, 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, or 123, or a sequence that exhibits at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity to any of SEQ ID NOS: 74, 75, 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, 123; and ii) a variable light chain set forth in any of SEQ ID NOS: 4, 9 or 11 or a sequence that exhibits at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity to any of SEQ ID NOS: 4, 9, or 11.

In other examples, provided herein are 104E modified anti-EGFR antibodies containing: i) a variable heavy chain set forth in any of SEQ ID NOS: 74, 75, 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, or 123, or a sequence that exhibits at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity to any of SEQ ID NOS: 74, 75, 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, 123; and ii) a variable light chain set forth in any of SEQ ID NOS: 125-127, or a sequence that exhibits at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity to any of SEQ ID NOS: 125-127.

In particular examples, provided herein are 104E modified anti-EGFR antibodies containing: i) a variable heavy chain set forth in SEQ ID NO: 74 or 75 or a sequence that exhibits at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity to any of SEQ ID NOS: 74 or 75; and ii) a variable light chain set forth in any of SEQ ID NOS: 125-127, or a sequence that exhibits at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity to any of SEQ ID NOS: 125-127.

In other particular examples, provided herein are 104E modified anti-EGFR antibodies containing additional modifications in both the variable heavy chain and variable light chain, whereby the anti-EGFR antibody contains: i) a variable heavy chain set forth in SEQ ID NO: 77 or 78, or a sequence that at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity to any of SEQ ID NOS: 77 or 78; and ii) a variable light chain set forth in any of SEQ ID NOS: 125-127, or a sequence that exhibits at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity to any of SEQ ID NOS: 125-127.

The modified anti-EGFR antibodies provided herein can be full-length IgG1 antibodies, or other subtype from among IgG2, IgG3 or IgG4. For example, the anti-EGFR antibodies can be full-length IgG1 antibodies containing a kappa light chain constant region (set forth in SEQ ID NO: 31 or 33) or an IgG1 heavy chain constant region set forth in any of SEQ ID NOS: 19-23). The heavy chain constant region also can be from an Ig class, such as IgG2 (set forth in SEQ ID NO: 24), IgG3 (set forth in SEQ ID NO: 25) or IgG4 (set forth in SEQ ID NO: 26). The light chain constant region also can be a human lambda light chain (set forth in SEQ ID NO: 32).

For example, provided herein are modified anti-EGFR antibodies that are full-length antibodies containing: i) a heavy chain variable having the sequence of amino acids set forth in any of SEQ ID NOS: 74, 75, 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, or 123, or a sequence that exhibits at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity to any of SEQ ID NOS: 74, 75, 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, or 123 containing the amino acid replacement 104E, and further containing the sequence of amino acids corresponding to an IgG1 constant region set forth in any of SEQ ID NOS: 19-23; and ii) a light chain. The light chain can contain the sequence of amino acids set forth in any of SEQ ID NOS: 4, 9 or 11 or a sequence that exhibits at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity to any of SEQ ID NOS: 4, 9, or 11, further containing a kappa light chain constant region set forth in any of SEQ ID NOS: 31, 33 or 34 or a lambda light chain constant region set forth in SEQ ID NO: 32 or variant thereof. For example, the light chain can have the sequence of amino acids set forth in SEQ ID NO: 3, 8, 10 or 13, or a sequence that exhibits at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity to any of SEQ ID NOS: 3, 8, 10 or 13. The light chain also can be a modified light chain variable domain having the sequence of amino acids set forth in any of SEQ ID NOS: 125-127, or a sequence that exhibits at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity to any of SEQ ID NOS: 125-127, further containing a sequence of amino acids corresponding to a kappa light chain constant region set forth in any of SEQ ID NOS: 31, 33 or 34 or a lambda light chain constant region set forth in SEQ ID NO: 32 or variant thereof.

In particular, provided herein are modified anti-EGFR antibodies that are full-length antibodies containing: i) a heavy chain set forth in any of SEQ ID NOS: 72, 76, 79, 82, 85, 88, 91, 94, 97, 100, 103, 106, 109, 112, 115, 118, or 121, or a sequence that exhibits at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity to any of SEQ ID NOS: 72, 76, 79, 82, 85, 88, 91, 94, 97, 100, 103, 106, 109, 112, 115, 118, or 121 containing the amino acid replacement 104E; and ii) a light chain. The light chain can have the sequence of amino acids set forth any of SEQ ID NOS: 3, 8, 10 or 13, or a sequence that exhibits at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity to any of SEQ ID NOS: 3, 8, 10 or 13. The light chain also can have the sequence of amino acids set forth in SEQ ID NO: 124, or a sequence that exhibits at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity to SEQ ID NO: 124.

Modified anti-EGFR antibodies provided herein also include antibody fragments, which are derivatives of full-length antibodies that contain less than the full sequence of the full-length antibodies but retain at least a portion of the specific binding abilities of the full-length antibody, such as the variable portions of the heavy and light chain. The antibody fragments also can include antigen-binding portions of an antibody that can be inserted into an antibody framework (e.g., chimeric antibodies) in order to retain the binding affinity of the parent antibody. Examples of antibody fragments include, but are not limited to, Fab, Fab′, F(ab′)₂, single-chain Fv (scFv), Fv, dsFv, diabody, Fd and Fd′ fragments, and other fragments, including modified fragments (see, for example, Methods in Molecular Biology, Vol. 207: Recombinant Antibodies for Cancer Therapy Methods and Protocols (2003); Chapter 1; p 3-25, Kipriyanov).

Antibody fragments can include multiple chains linked together, such as by disulfide bridges and can be produced recombinantly. Antibody fragments also can contain synthetic linkers, such as peptide linkers, to link two or more domains. Methods for generating antigen-binding fragments are well-known known in the art and can be used to modify any antibody provided herein. Fragments of antibody molecules can be generated, such as for example, by enzymatic cleavage. For example, upon protease cleavage by papain, a dimer of the heavy chain constant regions, the Fc domain, is cleaved from the two Fab regions (i.e., the portions containing the variable regions). Alternatively, pepsin cleavage can be used to prepare divalent F(ab′)₂ fragments of an antibody. Antibody fragments also can be generated synthetically or by recombinant DNA methods.

Single chain antibodies can be recombinantly engineered by joining a heavy chain variable region (V_{H}) and light chain variable region (V_{L}) of a specific antibody. The particular nucleic acid sequences for the variable regions can be cloned by standard molecular biology methods, such as, for example, by polymerase chain reaction (PCR) and other recombination nucleic acid technologies. Methods for producing scFvs are described, for example, by Whitlow and Filpula (1991) Methods, 2: 97-105; Bird et al. (1988) Science 242:423-426; Pack et al. (1993) Bio/Technology 11:1271-77; and U.S. Pat. Nos. 4,946,778, 5,840,300, 5,667,988, 5,658,727, 5,258,498).

Fragments of modified anti-EGFR antibodies provided herein, such as any described herein above, contain: i) a modified variable heavy chain set forth in any of SEQ ID NOS: 74, 75, 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, or 123, or an antigen-binding fragment or variant thereof that exhibits a sequence identity of at least 75%, 80%, 81%, 82%, 83%, 84%, 85%, 86%, 86%, 88%, 89%, 90%, 91%, 92%, 93%, 94%, 95%, 96%, 97%, 98%, 99% or more to any of SEQ ID NOS: 74, 75, 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, 123, or 125-127 and contains the amino acid replacement 104E; and ii) a variable light chain heavy domain set forth in any of SEQ ID NOS: 4, 9, 11, 15, 17 or 125-127, or an antigen-binding fragment or variant thereof that exhibits a sequence identity of at least 75%, 80%, 81%, 82%, 83%, 84%, 85%, 86%, 86%, 88%, 89%, 90%, 91%, 92%, 93%, 94%, 95%, 96%, 97%, 98%, 99% or more to any of SEQ ID NOS: 4, 9, 11, 15, 17 or 125-127. For example, examples of antibody fragments include, but are not limited to, Fab, Fab′, F(ab′)₂, single-chain Fv (scFv), Fv, dsFv, diabody, Fd and Fd′ fragments.

For example, such anti-EGFR antibodies can be Fab fragments (VH-CH1 and CL-CL). In such examples, the 104E variable heavy chain regions described above can further contain a heavy chain C_{H}1 constant region from an IgG1 (e.g., corresponding to amino acid residues 1-98 of any of SEQ ID NOS: 19-23) or other subtype or isotype (e.g., corresponding to amino acid residues 1-98 of any of SEQ ID NOS: 24-27). The variable light chain regions described above can further contain a kappa light chain constant region set forth in any of SEQ ID NOS: 31, 33 or 34 or a lambda light chain constant region set forth in SEQ ID NO: 32. For example, provided herein, are modified anti-EGFR Fab antibodies containing: i) a variable heavy chain set forth in any of SEQ ID NOS: 74, 75, 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, or 123, or a sequence that exhibits at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity to any of SEQ ID NOS: 74, 75, 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, or 123 containing the amino acid replacement 104E, and further containing the sequence of amino acids corresponding amino acids 1-98 of an IgG1 constant region set forth in any of SEQ ID NOS: 19-23 or variant thereof; and ii) a light chain having the sequence of amino acids set forth any of SEQ ID NOS: 3, 8, 10, 13 or 124, or a sequence that exhibits at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity to any of SEQ ID NOS: 3, 8, 10, 13 or 124.

In particular examples, the modified anti-EGFR antibody is a single chain antibody. A single chain antibody can be generated from the antigen-binding domain of any of the anti-EGFR antibodies provided herein. Methods for generating single chain antibodies using recombinant techniques are known in the art, such as those described in, for example, Marasco et al. (1993) Proc. Natl. Acad. Sci. USA 90:7889-7893, Whitlow and Filpula (1991) Methods, 2: 97-105; Bird et al. (1988) Science 242:423-426; Pack et al. (1993) Bio/Technology 11:1271-77; and U.S. Pat. Nos. 4,946,778, 5,840,300, 5,667,988, 5,658,727.

A single chain antibody can contain a light chain variable (V_{L}) domain or functional region thereof and a heavy chain variable (V_{H}) domain or functional region thereof of any anti-EGFR antibody or antigen-binding fragment thereof provided herein. In some examples, the V_{L} domain or functional region thereof of the single chain antibody contains a complementarity determining region 1 (CDR1), a complementarity determining region 2 (CDR2) and/or a complementarity determining region 3 (CDR3) of an anti-EGFR antibody, or antigen-binding fragment thereof, provided herein. In some examples, the V_{H} domain, or functional region thereof, of the single chain antibody contains a complementarity determining region 1 (CDR1), a complementarity determining region 2 (CDR2) and a complementarity determining region 3 (CDR3) of any anti-EGFR antibody, or antigen-binding fragment thereof, provided herein.

In some examples, the single chain antibody further contains a peptide linker. In such examples, a peptide linker can be located between the light chain variable domain (V_{L}) and the heavy chain variable domain (V_{H}). The single chain antibody can contain a peptide spacer, or linker, between the one or more domains of the antibody. For example, the light chain variable domain (V_{L}) of an antibody can be coupled to a heavy chain variable domain (V_{H}) via a flexible linker peptide. Generally, linker peptides are approximately 1-50 amino acids in length. The linkers used herein also can increase intracellular availability, serum stability, specificity and solubility or provide increased flexibility or relieve steric hindrance. Linking moieties are described, for example, in Huston et al. (1988) Proc Natl Acad Sci USA 85:5879-5883, Whitlow et al. (1993) Protein Engineering 6:989-995, and Newton et al., (1996) Biochemistry 35:545-553.

Various peptide linkers are well-known in the art and can be employed in the provided methods. A peptide linker can include a series of glycine residues (Gly) or Serine (Ser) residues. Exemplary polypeptide linkers are peptides having the amino acid sequences (Gly-Ser)ₙ, (GlyₘSer)ₙ or (SerₘGly)ₙ, in which m is 1 to 6, generally 1 to 4, and typically 2 to 4, and n is 1 to 30, or 1 to 10, and typically 1 to 4, with some glutamic acid (Glu) or lysine (Lys) residues dispersed throughout to increase solubility (see, e.g., International PCT application No. WO 96/06641, which provides exemplary linkers for use in conjugates). Exemplary peptide linkers include, but are not limited to peptides having the sequence (Gly₄Ser)₃ (SEQ ID NO: 46), GGSSRSSSSGGGGSGGGG (SEQ ID NO: 327), GSGRSGGGGSGGGGS (SEQ ID NO: 328), EGKSSGSGSESKST (SEQ ID NO: 329), EGKSSGSGSESKSTQ (SEQ ID NO: 330), EGKSSGSGSESKVD (SEQ ID NO: 331), GSTSGSGKSSEGKG (SEQ ID NO: 332), KESGSVSSEQLAQFRSLD (SEQ ID NO: 333), and ESGSVSSEELAFRSLD (SEQ ID NO: 334). Other suitable peptide linkers include any of those described in U.S. Pat. No. 4,751,180 or 4,935,233, which are hereby incorporated by reference.

2. Humanized Anti-EGFR Antibodies

Provided herein are human or humanized anti-EGFR antibodies. For example, any modified anti-EGFR containing a modified heavy chain and/or modified light chain as provided in subsection C.1 above, can be humanized. For example, humanization can be performed with reference to any of the anti-EGFR antibodies provided herein that contain a variable heavy chain set forth in SEQ ID NOS: 74, 75, 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, or 123 or a sequence that exhibits at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity to any of SEQ ID NOS: 74, 75, 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, or 123, and that contain 104E; and a variable light chain set forth in any of SEQ ID NOS: 4, 9, 11 or 124-127, or a sequence that exhibits at least 75%, 80%, 81%, 82%, 83%, 84%, 85%, 86%, 86%, 88%, 89%, 90%, 91%, 92%, 93%, 94%, 95%, 96%, 97%, 98%, 99% to any of SEQ ID NOS: 4, 9, 11 or 124-127, including any of the exemplary antibodies described above. Methods of humanization are well-known to the skilled artisan. Antibody humanization can be used to evolve mouse or other non-human antibodies into human antibodies. The resulting antibody contains an increase in human sequence and a reduction to an elimination of non-human (e.g., mouse) antibody sequence, while maintaining similar binding affinity and specificity as the starting antibody.

Methods for engineering or humanizing non-human or human antibodies can be used and are well-known in the art. Generally, a humanized or engineered antibody has one or more amino acid residues from a source which is non-human, e.g., but not limited to, mouse, rat, rabbit, non-human primate or other mammal. The human amino acid residues are imported thereto, and hence are often referred to as “import” residues, which are typically taken from an “import” variable, constant or other domain of a known human sequence. Known human Ig sequences are disclosed, e.g., ncbi.nlm nih.gov/entrez/query.fcgi; atcc.org/phage/hdb.html; sciquest.com/; www.abcam.com/; antibodyresource.com/onlinecomp.html; public.iastate.edu/.about.pedro/research_tools.html; mgen.uni-heidelberg.de/SD/IT/IT.html; whfreeman.com/immunology/CH05/kuby05.htm; library.thinkquest.org/12429/Immune/Antibody.html; hhmi.org/grants/lectures/1996/vlab/; path.cam.ac.uk/.about.mrc7/mikeimages.html; antibodyresource.com/; mcb.harvard.edu/BioLinks/Immunology.html.immunologylink.com/; pathbox.wustl.edu/.about.hcenter/index.html; biotech.ufl.edu/.about.hcl/; www.pebio.com/pa/340913/340913.html; nal.usda.gov/awic/pubs/antibody/; m.chime-u.ac.p/.about.yasuhito/Elisa.html; biodesign.com/table.asp; icnet.uk/axp/facs/davies/links.html; biotech.ufl.edu/.about.fccl/protocol.html; isac-net.org/sites_geo.html; aximtl.imt.uni-marburg.de/.about.rek/AEPStart.html; baserv.uci.kun.nLabout.jraats/linksl.html; recab.uni-hd.de/immuno bme.nwvu.edu/; mrc-cpe.cam.ac.uk/imt-doc/public/INTRO.html; ibt.unam.mx/vir/V_mice.html; imgt.cnusc.fr:8104/; biochem.ucl.ac.uk/.about.martin/abs/index.html; antibody.bath.ac.uk/; abgen.cvm.tamu.edu/lab/wwwabgen.html; unizh.ch/.about.honegger/AHOseminar/Slide01.html; www.cryst.bbk.ac.uk/.about.ubcg07s/; nimr mrc.ac.uk/CC/ccaewg/ccaewg.htm; path.cam.ac.uk/.about.mrc7/humanisation/TAHHP.html; ibt.unam.mx/vir/structure/sta_taim.html; biosci.missouri.edu/smithgp/index.html; cryst.bioc.cam.ac.uk/.about.fmolina/Web-pages/Pept/spottech.html; jerini.de/frproducts.htm; patents.ibm.con/ibm.html; Kabat et al. Sequences of Proteins of Immunological Interest, U.S. Dept. Health (1983). Such imported sequences can be used to reduce immunogenicity or reduce, enhance or modify binding, affinity, on-rate, off-rate, avidity, specificity, half-life, or any other suitable characteristic, as known in the art. Generally part or all of the original non-human or human CDR sequences are maintained while the non-human sequences of the variable regions (e.g., framework regions) and constant regions are replaced with human sequences or other amino acids.

Antibodies also can optionally be humanized with retention of high affinity for the antigen and other favorable biological properties. To achieve this goal, humanized antibodies can be optionally prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved.

In general, the CDR residues are directly and most substantially involved in influencing antigen binding. Hence, the CDR residues are not generally targeted for humanization. Humanization or engineering of antibodies can be performed using any known method, such as but not limited to, those described in Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539; 4,816,567, PCT/: US98/16280, US96/18978, US91/09630, US91/05939, US94/01234, GB89/01344, GB91/01134, GB92/01755; WO90/14443, WO90/14424, WO90/14430, EP 229246, each entirely incorporated herein by reference, including references cited therein.

For example, antibody humanization can be performed, for example, by synthesizing a combinatorial library containing the six CDRs of a target antibody to be humanized (e.g., the CDRs of any of the modified anti-EGFR antibodies set forth above) fused in frame to a pool of individual human frameworks. For example, the CDRs can be derived from any one or more of the CDRH1 (amino acid residues 26-35, according to AbM definition, or amino acid residues 31-35, according to Kabat definition), CDRH2 (amino acid residues 50-65) or CDRH3 (amino acid residues 95-102) set forth in any of SEQ ID NOS: 74, 75, 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, or 123 and/or can be derived from any one or more of the CDRL1 (amino acid residues 24-34), CDRL2 (amino acid residues 50-56) or CDRL3 (amino acid residues 89-97) set forth in any of SEQ ID NOS: 4, 9, 11 or 125-127. A human framework library that contains genes representative of all known heavy and light chain human germline genes can be utilized. The resulting combinatorial libraries can then be screened for conditional binding to antigens of interest as described herein. This approach can allow for the selection of the most favorable combinations of fully human frameworks in terms of maintaining the affinity and conditional binding activity of the parental antibody. Humanized antibodies can then be further optimized by a variety of techniques.

The number of amino acid substitutions or replacements a skilled artisan can make to effect humanization depends on many factors, including those described above. In general, the number of amino acid replacements (substitutions), insertions or deletions for an anti-EGFR antibody, fragment or variant will not be more than 40, 30, 20, 19, 18, 17, 16, 15, 14, 13, 12, 11, 10, 9, 8, 7, 6, 5, 4, 3, 2, 1, such as 1-30 or any range or value therein, as specified herein Amino acids in an anti-EGFR antibody that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to binding to EGFR using any of the methods described herein. Sites that are critical for antibody binding can also be identified by structural analysis such as crystallization, nuclear magnetic resonance or photoaffinity labeling (Smith, et al., J. Mol. Biol. 224:899-904 (1992) and de Vos, et al., Science 255:306-312 (1992)).

Humanized antibodies provided herein also can be generated based on a known humanized backbone or reference anti-EGFR antibody. For example, the known humanized antibodies H225 (VH set forth in SEQ ID NO: 14 and VL set forth in SEQ ID NO: 15) or Hu225 (VH set forth in SEQ ID NO: 16 or VL set forth in SEQ ID NO: 17) can be used as an unmodified or reference anti-EGFR antibody into which the 104E amino acid replacement, and optionally one or more other amino acid replacement(s), is/are introduced. For example, humanized Cetuximab anti-EGFR antibodies, for example H225, with a variable heavy chain set forth in SEQ ID NO: 14 and a variable light chain set forth in SEQ ID NO: 15, and Hu225, with a variable heavy chain set forth in SEQ ID NO: 16 and a variable light chain set forth in SEQ ID NO: 17, can be modified by site directed mutagenesis to yield a humanized 104E (E-h) antibody and variants thereof.

In other cases, any of the humanized anti-EGFR antibodies described in U.S. patent application Ser. No. 13/815,553 can be used as an unmodified or reference anti-EGFR antibody into which the 104E amino acid replacement, and optionally one or more other amino acid replacement(s), is/are introduced. Humanized antibodies that can be used as an unmodified or reference anti-EGFR antibody include, but are not limited to, any of the humanized antibodies containing the amino acid replacement 104D set forth in Table 11 (e.g., designated DP-h1-h10, DP-h12-h14 or FDP-h1-h21). For example, exemplary humanized reference or backbone antibodies are the anti-EGFR antibody designated Y104D/Q111P (DP-h07) (e.g., having a heavy chain set forth in SEQ ID NO: 55 and light chain set forth in SEQ ID NO: 181) or the anti-EGFR antibody designated T030F/Y104D/Q111P (FDP-h03) (e.g., having a heavy chain set forth in SEQ ID NO: 65 and light chain set forth in SEQ ID NO: 258) or the anti-EGFR antibody designated Y104D (D-h07) (e.g., having a heavy chain set forth in SEQ ID NO: 57 and a light chain set forth in SEQ ID NO: 181). Any of such unmodified or reference humanized sequences can be subjected to site directed mutagenesis to generate humanized anti-EGFR antibodies containing the amino acid replacement 104E, and optionally one or more other amino acid replacement.

Non-limiting examples of 104E humanized clones are set forth in Table 10, which sets forth the SEQ ID NO of the heavy and light chains of each clone.

TABLE 10
Exemplary Humanized 104E Clones

| | HEAVY CHAIN | | | | | | LIGHT CHAIN | | | | | | |
|---|---|---|---|---|---|---|---|---|---|---|---|---|---|
| | | | Variable | | Variable | | | | Variable | | Variable | Variable | |
| | Full length | | region^{a} | | region^{b} | | Full length | | region^{c} | | region^{d} | region^{e} | |
| | nucleic | amino | nucleic | amino | nucleic | amino | nucleic | amino | nucleic | amino | amino | nucleic | amino |
| | acid | acid | acid | acid | acid | acid | acid | acid | acid | acid | acid | acid | acid |
| cetuximab | 48 | 6 | 49 | 7 | | 2 | 50 | 8 | 51 | 9 | 4 | | 11 |
| E-h07 | 58 | 59 | 60 | 61 | 62 | 63 | 180 | 181 | 182 | 183 | 184 | 185 | 186 |
| EP-h01 | 128 | 129 | 130 | 131 | 132 | 133 | 152 | 153 | 154 | 155 | 156 | 157 | 158 |
| EP-h02 | 128 | 129 | 130 | 131 | 132 | 133 | 159 | 160 | 161 | 162 | 163 | 164 | 165 |
| EP-h03 | 134 | 135 | 136 | 137 | 138 | 139 | 152 | 153 | 154 | 155 | 156 | 157 | 158 |
| EP-h04 | 128 | 129 | 130 | 131 | 132 | 133 | 166 | 167 | 168 | 169 | 170 | 171 | 172 |
| EP-h05 | 128 | 129 | 130 | 131 | 132 | 133 | 173 | 174 | 175 | 176 | 177 | 178 | 179 |
| EP-h06 | 128 | 129 | 130 | 131 | 132 | 133 | 180 | 181 | 182 | 183 | 184 | 185 | 186 |
| EP-h07 | 134 | 135 | 136 | 137 | 138 | 139 | 180 | 181 | 182 | 183 | 184 | 185 | 186 |
| EP-h08 | 128 | 129 | 130 | 131 | 132 | 133 | 187 | 188 | 189 | 190 | 191 | 192 | 193 |
| EP-h09 | 140 | 141 | 142 | 143 | 144 | 145 | 180 | 181 | 182 | 183 | 184 | 185 | 186 |
| EP-h10 | 146 | 147 | 148 | 149 | 150 | 151 | 194 | 195 | 196 | 197 | 198 | 199 | 200 |
| EP-h12 | 140 | 141 | 142 | 143 | 144 | 145 | 194 | 195 | 196 | 197 | 198 | 199 | 200 |
| EP-h13 | 146 | 147 | 148 | 149 | 150 | 151 | 201 | 202 | 203 | 204 | 205 | 206 | 207 |
| EP-h14 | 140 | 141 | 142 | 143 | 144 | 145 | 201 | 202 | 203 | 204 | 205 | 206 | 207 |
| FEP-h01 | 208 | 209 | 210 | 211 | 212 | 213 | 250 | 251 | 252 | 253 | 254 | 255 | 256 |
| FEP-h02 | 214 | 215 | 216 | 217 | 218 | 219 | 250 | 251 | 252 | 253 | 254 | 255 | 256 |
| FEP-h03 | 220 | 221 | 222 | 223 | 224 | 225 | 257 | 258 | 259 | 260 | 261 | 262 | 263 |
| FEP-h04 | 226 | 227 | 228 | 229 | 230 | 231 | 257 | 258 | 259 | 260 | 261 | 262 | 263 |
| FEP-h05 | 232 | 233 | 234 | 235 | 236 | 237 | 264 | 265 | 266 | 267 | 268 | 269 | 270 |
| FEP-h06 | 238 | 239 | 240 | 241 | 242 | 243 | 271 | 272 | 273 | 274 | 275 | 276 | 277 |
| FEP-h07 | 220 | 221 | 222 | 223 | 224 | 225 | 271 | 272 | 273 | 274 | 275 | 276 | 277 |
| FEP-h08 | 226 | 227 | 228 | 229 | 230 | 231 | 271 | 272 | 273 | 274 | 275 | 276 | 277 |
| FEP-h09 | 232 | 233 | 234 | 235 | 236 | 237 | 278 | 279 | 280 | 281 | 282 | 283 | 284 |
| FEP-h10 | 244 | 245 | 246 | 247 | 248 | 249 | 278 | 279 | 280 | 281 | 282 | 283 | 284 |
| FEP-h11 | 220 | 221 | 222 | 223 | 224 | 225 | 278 | 279 | 280 | 281 | 282 | 283 | 284 |
| FEP-h12 | 226 | 227 | 228 | 229 | 230 | 231 | 278 | 279 | 280 | 281 | 282 | 283 | 284 |
| FEP-h13 | 232 | 233 | 234 | 235 | 236 | 237 | 285 | 286 | 287 | 288 | 289 | 290 | 291 |
| FEP-h14 | 244 | 245 | 246 | 247 | 248 | 249 | 285 | 286 | 287 | 288 | 289 | 290 | 291 |
| FEP-h15 | 220 | 221 | 222 | 223 | 224 | 225 | 285 | 286 | 287 | 288 | 289 | 290 | 291 |
| FEP-h16 | 226 | 227 | 228 | 229 | 230 | 231 | 285 | 286 | 287 | 288 | 289 | 290 | 291 |
| FEP-h17 | 232 | 233 | 234 | 235 | 236 | 237 | 292 | 293 | 294 | 295 | 296 | 297 | 298 |
| FEP-h18 | 244 | 245 | 246 | 247 | 248 | 249 | 299 | 300 | 301 | 302 | 303 | 304 | 305 |
| FEP-h19 | 208 | 209 | 210 | 211 | 212 | 213 | 299 | 300 | 301 | 302 | 303 | 304 | 305 |
| FEP-h20 | 208 | 209 | 210 | 211 | 212 | 213 | 278 | 279 | 280 | 281 | 282 | 283 | 284 |
| FEP-h21 | 208 | 209 | 210 | 211 | 212 | 213 | 285 | 286 | 287 | 288 | 289 | 290 | 291 |

Variable region^{a-b}: derived from known cetuximab heavy chain variable region (see Section B)
Variable region^{c-e}: derived from known cetuximab light chain variable region (see Section B)

Exemplary humanized anti-EGFR antibodies provided herein include any containing a variable heavy chain (VH) and variable light chain (VL) having a sequence of amino acids set forth as:

the VH set forth in SEQ ID NO: 61 or 63 or a sequence of amino acids that exhibits at least 85% sequence identity to SEQ ID NO: 61 or 63, and the VL set forth in SEQ ID NO: 183, 184 or 186 or a sequence of amino acids that exhibits at least 85% sequence identity to SEQ ID NO: 183, 184 or 186;

the VH set forth in SEQ ID NO: 131 or 133 or a sequence of amino acids that exhibits at least 85% sequence identity to SEQ ID NO: 131 or 133, and the VL set forth in SEQ ID NO: 155, 156 or 158 or a sequence of amino acids that exhibits at least 85% sequence identity to SEQ ID NO: 155, 156 or 158;

the VH set forth in SEQ ID NO: 131 or 133 or a sequence of amino acids that exhibits at least 85% sequence identity to SEQ ID NO: 131 or 133, and the VL set forth in SEQ ID NO: 162, 163 or 165 or a sequence of amino acids that exhibits at least 85% sequence identity to SEQ ID NO: 162, 163 or 165;

the VH set forth in SEQ ID NO: 137 or 139 or a sequence of amino acids that exhibits at least 85% sequence identity to SEQ ID NO: 137 or 139, and the VL set forth in SEQ ID NO: 155, 156 or 158 or a sequence of amino acids that exhibits at least 85% sequence identity to SEQ ID NO: 155, 156 or 158;

the VH set forth in SEQ ID NO: 131 or 133 or a sequence of amino acids that exhibits at least 85% sequence identity to SEQ ID NO: 131 or 133, and the VL set forth in SEQ ID NO: 169, 170 or 172 or a sequence of amino acids that exhibits at least 85% sequence identity to SEQ ID NO: 169, 170 or 172;

the VH set forth in SEQ ID NO: 131 or 133 or a sequence of amino acids that exhibits at least 85% sequence identity to SEQ ID NO: 131, and the VL set forth in SEQ ID NO: 176, 177 or 179 or a sequence of amino acids that exhibits at least 85% sequence identity to SEQ ID NO: 176, 177 or 179;

the VH set forth in SEQ ID NO: 131 or 133 or a sequence of amino acids that exhibits at least 85% sequence identity to SEQ ID NO: 131 or 133 and the VL set forth in SEQ ID NO: 183, 184 or 186 or a sequence of amino acids that exhibits at least 85% sequence identity to SEQ ID NO: 183, 184 or 186;

the VH set forth in SEQ ID NO: 137 or 139 or a sequence of amino acids that exhibits at least 85% sequence identity to SEQ ID NO: 137 or 139, and the VL set forth in SEQ ID NO: 183, 184 or 186 or a sequence of amino acids that exhibits at least 85% sequence identity to SEQ ID NO: 183, 184 or 186;

the VH set forth in SEQ ID NO: 131 or 133 or a sequence of amino acids that exhibits at least 85% sequence identity to SEQ ID NO: 131 or 133, and the VL set forth in SEQ ID NO: 190, 191 or 193 or a sequence of amino acids that exhibits at least 85% sequence identity to SEQ ID NO: 190, 191 or 193;

the VH set forth in SEQ ID NO: 143 or 145 or a sequence of amino acids that exhibits at least 85% sequence identity to SEQ ID NO: 143 or 145, and the VL set forth in SEQ ID NO: 183, 184 or 186 or a sequence of amino acids that exhibits at least 85% sequence identity to SEQ ID NO: 183, 184 or 186;

the VH set forth in SEQ ID NO: 149 or 151 or a sequence of amino acids that exhibits at least 85% sequence identity to SEQ ID NO: 149 or 151, and the VL set forth in SEQ ID NO: 197, 198 or 200 or a sequence of amino acids that exhibits at least 85% sequence identity to SEQ ID NO: 197, 198 or 200;

the VH set forth in SEQ ID NO: 143 or 145 or a sequence of amino acids that exhibits at least 85% sequence identity to SEQ ID NO: 143 or 145, and the VL set forth in SEQ ID NO: 197, 198 or 200 or a sequence of amino acids that exhibits at least 85% sequence identity to SEQ ID NO: 197, 198 or 200;

the VH set forth in SEQ ID NO: 149 or 151 or a sequence of amino acids that exhibits at least 85% sequence identity to SEQ ID NO: 149 or 151, and the VL set forth in SEQ ID NO: 204, 205 or 207 or a sequence of amino acids that exhibits at least 85% sequence identity to SEQ ID NO: 204, 205 or 207;

the VH set forth in SEQ ID NO: 143 or 145 or a sequence of amino acids that exhibits at least 85% sequence identity to SEQ ID NO: 143 or 145, and the VL set forth in SEQ ID NO: 204, 205 or 207 or a sequence of amino acids that exhibits at least 85% sequence identity to SEQ ID NO: 204, 205 or 207;

the VH set forth in SEQ ID NO: 211 or 213 or a sequence of amino acids that exhibits at least 85% sequence identity to SEQ ID NO: 211 or 213, and the VL set forth in SEQ ID NO: 253, 254 or 256 or a sequence of amino acids that exhibits at least 85% sequence identity to SEQ ID NO: 253, 254 or 256;

the VH set forth in SEQ ID NO: 217 or 219 or a sequence of amino acids that exhibits at least 85% sequence identity to SEQ ID NO: 217 or 219, and the VL set forth in SEQ ID NO: 253, 254 or 256 or a sequence of amino acids that exhibits at least 85% sequence identity to SEQ ID NO: 253, 254 or 256;

the VH set forth in SEQ ID NO: 223 or 225 or a sequence of amino acids that exhibits at least 85% sequence identity to SEQ ID NO: 223 or 225, and the VL set forth in SEQ ID NO: 260, 261 or 263 or a sequence of amino acids that exhibits at least 85% sequence identity to SEQ ID NO: 260, 261 or 263;

the VH set forth in SEQ ID NO: 229 or 231 or a sequence of amino acids that exhibits at least 85% sequence identity to SEQ ID NO: 229 or 231, and the VL set forth in SEQ ID NO: 260, 261 or 263 or a sequence of amino acids that exhibits at least 85% sequence identity to SEQ ID NO: 260, 261 or 263;

the VH set forth in SEQ ID NO: 235 or 237 or a sequence of amino acids that exhibits at least 85% sequence identity to SEQ ID NO: 235 or 237, and the VL set forth in SEQ ID NO: 267, 268 or 270 or a sequence of amino acids that exhibits at least 85% sequence identity to SEQ ID NO: 267, 268 or 270;

the VH set forth in SEQ ID NO: 241 or 243 or a sequence of amino acids that exhibits at least 85% sequence identity to SEQ ID NO: 241 or 243, and the VL set forth in SEQ ID NO: 274, 275 or 277 or a sequence of amino acids that exhibits at least 85% sequence identity to SEQ ID NO: 274, 275 or 277;

the VH set forth in SEQ ID NO: 223 or 225 or a sequence of amino acids that exhibits at least 85% sequence identity to SEQ ID NO: 223 or 225, and the VL set forth in SEQ ID NO: 274, 275 or 277 or a sequence of amino acids that exhibits at least 85% sequence identity to SEQ ID NO: 274, 275 or 277;

the VH set forth in SEQ ID NO: 229 or 231 or a sequence of amino acids that exhibits at least 85% sequence identity to SEQ ID NO: 229 or 231, and the VL set forth in SEQ ID NO: 274, 275 or 277 or a sequence of amino acids that exhibits at least 85% sequence identity to SEQ ID NO: 274, 275 or 277;

the VH set forth in SEQ ID NO: 235 or 237 or a sequence of amino acids that exhibits at least 85% sequence identity to SEQ ID NO: 235 or 237, and the VL set forth in SEQ ID NO: 281, 282 or 284 or a sequence of amino acids that exhibits at least 85% sequence identity to SEQ ID NO: 281, 282 or 284;

the VH set forth in SEQ ID NO: 247 or 249 or a sequence of amino acids that exhibits at least 85% sequence identity to SEQ ID NO: 247 or 249, and the VL set forth in SEQ ID NO: 281, 282 or 284 or a sequence of amino acids that exhibits at least 85% sequence identity to SEQ ID NO: 281, 282 or 284;

the VH set forth in SEQ ID NO: 223 or 225 or a sequence of amino acids that exhibits at least 85% sequence identity to SEQ ID NO: 223 or 225, and the VL set forth in SEQ ID NO: 281, 282 or 284 or a sequence of amino acids that exhibits at least 85% sequence identity to SEQ ID NO: 281, 282 or 284;

the VH set forth in SEQ ID NO: 229 or 231 or a sequence of amino acids that exhibits at least 85% sequence identity to SEQ ID NO: 229 or 231, and the VL set forth in SEQ ID NO: 281, 282 or 284 or a sequence of amino acids that exhibits at least 85% sequence identity to SEQ ID NO: 281, 282 or 284;

the VH set forth in SEQ ID NO: 235 or 237 or a sequence of amino acids that exhibits at least 85% sequence identity to SEQ ID NO: 235 or 237, and the VL set forth in SEQ ID NO: 288, 289 or 291 or a sequence of amino acids that exhibits at least 85% sequence identity to SEQ ID NO: 288, 289 or 291;

the VH set forth in SEQ ID NO: 247 or 249 or a sequence of amino acids that exhibits at least 85% sequence identity to SEQ ID NO: 247 or 249, and the VL set forth in SEQ ID NO: 288, 289 or 291 or a sequence of amino acids that exhibits at least 85% sequence identity to SEQ ID NO: 288, 289 or 291;

the VH set forth in SEQ ID NO: 223 or 225 or a sequence of amino acids that exhibits at least 85% sequence identity to SEQ ID NO: 223 or 225, and the VL set forth in SEQ ID NO: 288, 289 or 291 or a sequence of amino acids that exhibits at least 85% sequence identity to SEQ ID NO: 288, 289 or 291;

the VH set forth in SEQ ID NO: 229 or 231 or a sequence of amino acids that exhibits at least 85% sequence identity to SEQ ID NO: 229 or 231, and the VL set forth in SEQ ID NO: 288, 289 or 291 or a sequence of amino acids that exhibits at least 85% sequence identity to SEQ ID NO: 288, 289 or 291;

the VH set forth in SEQ ID NO: 235 or 237 or a sequence of amino acids that exhibits at least 85% sequence identity to SEQ ID NO: 235 or 237, and the VL set forth in SEQ ID NO: 295, 296 or 298 or a sequence of amino acids that exhibits at least 85% sequence identity to SEQ ID NO: 295, 296 or 298;

the VH set forth in SEQ ID NO: 247 or 249 or a sequence of amino acids that exhibits at least 85% sequence identity to SEQ ID NO: 247 or 249, and the VL set forth in SEQ ID NO: 302, 303 or 305 or a sequence of amino acids that exhibits at least 85% sequence identity to SEQ ID NO: 302, 303 or 305;

the VH set forth in SEQ ID NO: 211 or 213 or a sequence of amino acids that exhibits at least 85% sequence identity to SEQ ID NO: 211 or 213, and the VL set forth in SEQ ID NO: 302, 303 or 305 or a sequence of amino acids that exhibits at least 85% sequence identity to SEQ ID NO: 302, 303 or 305;

the VH set forth in SEQ ID NO: 211 or 213 or a sequence of amino acids that exhibits at least 85% sequence identity to SEQ ID NO: 211 or 213, and the VL set forth in SEQ ID NO: 281, 282 or 284 or a sequence of amino acids that exhibits at least 85% sequence identity to SEQ ID NO: 281, 282 or 284;

the VH set forth in SEQ ID NO: 211 or 213 or a sequence of amino acids that exhibits at least 85% sequence identity to SEQ ID NO: 211 or 213, and the VL set forth in SEQ ID NO: 288, 289 or 291 or a sequence of amino acids that exhibits at least 85% sequence identity to SEQ ID NO: 288, 289 or 291;

Any of the above anti-EGFR antibodies can further contain a heavy chain constant region or light chain constant region, or a portion thereof. The constant region can be any immunoglobulin class (e.g., IgG, IgM, IgD, IgE, IgA and IgY), any subclass (e.g., IgG1, IgG2, IgG3, IgG4, IgA1 and IgA2) or sub-subclass (e.g., IgG2a and IgG2b). In particular examples, the antibodies provided herein can be full-length antibodies further containing a constant region from an IgG1 antibody, or other subtype from among IgG2, IgG3 or IgG4. For example, the anti-EGFR antibodies can be full-length IgG1 antibodies containing a kappa light chain constant region (set forth in SEQ ID NO: 31 or 33) or an IgG1 heavy chain constant region set forth in any of SEQ ID NOS: 19-23). The heavy chain constant region also can be from an Ig

class, such as IgG2 (set forth in SEQ ID NO: 24), IgG3 (set forth in SEQ ID NO: 25) or IgG4 (set forth in SEQ ID NO: 26). The light chain constant region also can be a human lambda light chain (set forth in SEQ ID NO: 32).

Exemplary humanized anti-EGFR antibodies provided herein include any containing a heavy and light chain having a sequence of amino acids set forth as:

the heavy chain set forth in SEQ ID NO: 59 or a sequence of amino acids that exhibits at least 85% sequence identity to SEQ ID NO: 59, and the variable light chain set forth in SEQ ID NO: 181 or a sequence of amino acids that exhibits at least 85% sequence identity to SEQ ID NO: 181;

the heavy chain set forth in SEQ ID NO: 129 or a sequence of amino acids that exhibits at least 85% sequence identity to SEQ ID NO: 129, and the variable light chain set forth in SEQ ID NO: 153 or a sequence of amino acids that exhibits at least 85% sequence identity to SEQ ID NO: 153;

the heavy chain set forth in SEQ ID NO: 129 or a sequence of amino acids that exhibits at least 85% sequence identity to SEQ ID NO: 129, and the variable light chain set forth in SEQ ID NO: 160 or a sequence of amino acids that exhibits at least 85% sequence identity to SEQ ID NO: 160;

the heavy chain set forth in SEQ ID NO: 135 or a sequence of amino acids that exhibits at least 85% sequence identity to SEQ ID NO: 135, and the variable light chain set forth in SEQ ID NO: 153 or a sequence of amino acids that exhibits at least 85% sequence identity to SEQ ID NO: 153;

the heavy chain set forth in SEQ ID NO: 129 or a sequence of amino acids that exhibits at least 85% sequence identity to SEQ ID NO: 129, and the variable light chain set forth in SEQ ID NO: 167 or a sequence of amino acids that exhibits at least 85% sequence identity to SEQ ID NO: 167;

the heavy chain set forth in SEQ ID NO: 129 or a sequence of amino acids that exhibits at least 85% sequence identity to SEQ ID NO: 129, and the variable light chain set forth in SEQ ID NO: 174 or a sequence of amino acids that exhibits at least 85% sequence identity to SEQ ID NO: 174;

the heavy chain set forth in SEQ ID NO: 129 or a sequence of amino acids that exhibits at least 85% sequence identity to SEQ ID NO: 129, and the variable light chain set forth in SEQ ID NO: 181 or a sequence of amino acids that exhibits at least 85% sequence identity to SEQ ID NO: 181;

the heavy chain set forth in SEQ ID NO: 135 or a sequence of amino acids that exhibits at least 85% sequence identity to SEQ ID NO: 135, and the variable light chain set forth in SEQ ID NO: 181 or a sequence of amino acids that exhibits at least 85% sequence identity to SEQ ID NO: 181;

the heavy chain set forth in SEQ ID NO: 129 or a sequence of amino acids that exhibits at least 85% sequence identity to SEQ ID NO: 129, and the variable light chain set forth in SEQ ID NO: 188 or a sequence of amino acids that exhibits at least 85% sequence identity to SEQ ID NO: 188;

the heavy chain set forth in SEQ ID NO: 141 or a sequence of amino acids that exhibits at least 85% sequence identity to SEQ ID NO: 141, and the variable light chain set forth in SEQ ID NO: 181 or a sequence of amino acids that exhibits at least 85% sequence identity to SEQ ID NO: 181;

the heavy chain set forth in SEQ ID NO: 147 or a sequence of amino acids that exhibits at least 85% sequence identity to SEQ ID NO: 147, and the variable light chain set forth in SEQ ID NO: 195 or a sequence of amino acids that exhibits at least 85% sequence identity to SEQ ID NO: 195;

the heavy chain set forth in SEQ ID NO: 141 or a sequence of amino acids that exhibits at least 85% sequence identity to SEQ ID NO: 141, and the variable light chain set forth in SEQ ID NO: 195 or a sequence of amino acids that exhibits at least 85% sequence identity to SEQ ID NO: 195;

the heavy chain set forth in SEQ ID NO: 147 or a sequence of amino acids that exhibits at least 85% sequence identity to SEQ ID NO: 147, and the variable light chain set forth in SEQ ID NO: 202 or a sequence of amino acids that exhibits at least 85% sequence identity to SEQ ID NO: 202;

the heavy chain set forth in SEQ ID NO: 141 or a sequence of amino acids that exhibits at least 85% sequence identity to SEQ ID NO: 141, and the variable light chain set forth in SEQ ID NO: 202 or a sequence of amino acids that exhibits at least 85% sequence identity to SEQ ID NO: 202;

the heavy chain set forth in SEQ ID NO: 209 or a sequence of amino acids that exhibits at least 85% sequence identity to SEQ ID NO: 209, and the variable light chain set forth in SEQ ID NO: 251 or a sequence of amino acids that exhibits at least 85% sequence identity to SEQ ID NO: 251;

the heavy chain set forth in SEQ ID NO: 215 or a sequence of amino acids that exhibits at least 85% sequence identity to SEQ ID NO: 215, and the variable light chain set forth in SEQ ID NO: 251 or a sequence of amino acids that exhibits at least 85% sequence identity to SEQ ID NO: 251;

the heavy chain set forth in SEQ ID NO: 221 or a sequence of amino acids that exhibits at least 85% sequence identity to SEQ ID NO: 221, and the variable light chain set forth in SEQ ID NO: 258 or a sequence of amino acids that exhibits at least 85% sequence identity to SEQ ID NO: 258;

the heavy chain set forth in SEQ ID NO: 227 or a sequence of amino acids that exhibits at least 85% sequence identity to SEQ ID NO: 227, and the variable light chain set forth in SEQ ID NO: 258 or a sequence of amino acids that exhibits at least 85% sequence identity to SEQ ID NO: 258;

the heavy chain set forth in SEQ ID NO: 233 or a sequence of amino acids that exhibits at least 85% sequence identity to SEQ ID NO: 233, and the variable light chain set forth in SEQ ID NO: 265 or a sequence of amino acids that exhibits at least 85% sequence identity to SEQ ID NO: 265;

the heavy chain set forth in SEQ ID NO: 239 or a sequence of amino acids that exhibits at least 85% sequence identity to SEQ ID NO: 239, and the variable light chain set forth in SEQ ID NO: 272 or a sequence of amino acids that exhibits at least 85% sequence identity to SEQ ID NO: 272;

the heavy chain set forth in SEQ ID NO: 221 or a sequence of amino acids that exhibits at least 85% sequence identity to SEQ ID NO: 221, and the variable light chain set forth in SEQ ID NO: 272 or a sequence of amino acids that exhibits at least 85% sequence identity to SEQ ID NO: 272;

the heavy chain set forth in SEQ ID NO: 227 or a sequence of amino acids that exhibits at least 85% sequence identity to SEQ ID NO: 227, and the variable light chain set forth in SEQ ID NO: 272 or a sequence of amino acids that exhibits at least 85% sequence identity to SEQ ID NO: 272;

the heavy chain set forth in SEQ ID NO: 233 or a sequence of amino acids that exhibits at least 85% sequence identity to SEQ ID NO: 233, and the variable light chain set forth in SEQ ID NO: 279 or a sequence of amino acids that exhibits at least 85% sequence identity to SEQ ID NO: 279;

the heavy chain set forth in SEQ ID NO: 245 or a sequence of amino acids that exhibits at least 85% sequence identity to SEQ ID NO: 245, and the variable light chain set forth in SEQ ID NO: 279 or a sequence of amino acids that exhibits at least 85% sequence identity to SEQ ID NO: 279;

the heavy chain set forth in SEQ ID NO: 221 or a sequence of amino acids that exhibits at least 85% sequence identity to SEQ ID NO: 221, and the variable light chain set forth in SEQ ID NO: 279 or a sequence of amino acids that exhibits at least 85% sequence identity to SEQ ID NO: 279;

the heavy chain set forth in SEQ ID NO: 227 or a sequence of amino acids that exhibits at least 85% sequence identity to SEQ ID NO: 227, and the variable light chain set forth in SEQ ID NO: 279 or a sequence of amino acids that exhibits at least 85% sequence identity to SEQ ID NO: 279;

the heavy chain set forth in SEQ ID NO: 233 or a sequence of amino acids that exhibits at least 85% sequence identity to SEQ ID NO: 233, and the variable light chain set forth in SEQ ID NO: 286 or a sequence of amino acids that exhibits at least 85% sequence identity to SEQ ID NO: 286;

the heavy chain set forth in SEQ ID NO: 245 or a sequence of amino acids that exhibits at least 85% sequence identity to SEQ ID NO: 245, and the variable light chain set forth in SEQ ID NO: 286 or a sequence of amino acids that exhibits at least 85% sequence identity to SEQ ID NO: 286;

the heavy chain set forth in SEQ ID NO: 221 or a sequence of amino acids that exhibits at least 85% sequence identity to SEQ ID NO: 221, and the variable light chain set forth in SEQ ID NO: 286 or a sequence of amino acids that exhibits at least 85% sequence identity to SEQ ID NO: 286;

the heavy chain set forth in SEQ ID NO: 227 or a sequence of amino acids that exhibits at least 85% sequence identity to SEQ ID NO: 227, and the variable light chain set forth in SEQ ID NO: 286 or a sequence of amino acids that exhibits at least 85% sequence identity to SEQ ID NO: 286;

the heavy chain set forth in SEQ ID NO: 233 or a sequence of amino acids that exhibits at least 85% sequence identity to SEQ ID NO: 233, and the variable light chain set forth in SEQ ID NO: 293 or a sequence of amino acids that exhibits at least 85% sequence identity to SEQ ID NO: 293;

the heavy chain set forth in SEQ ID NO: 245 or a sequence of amino acids that exhibits at least 85% sequence identity to SEQ ID NO: 245, and the variable light chain set forth in SEQ ID NO: 300 or a sequence of amino acids that exhibits at least 85% sequence identity to SEQ ID NO: 300;

the heavy chain set forth in SEQ ID NO: 209 or a sequence of amino acids that exhibits at least 85% sequence identity to SEQ ID NO: 209, and the variable light chain set forth in SEQ ID NO: 300 or a sequence of amino acids that exhibits at least 85% sequence identity to SEQ ID NO: 300;

the heavy chain set forth in SEQ ID NO: 209 or a sequence of amino acids that exhibits at least 85% sequence identity to SEQ ID NO: 209, and the variable light chain set forth in SEQ ID NO: 279 or a sequence of amino acids that exhibits at least 85% sequence identity to SEQ ID NO: 279;

the heavy chain set forth in SEQ ID NO: 209 or a sequence of amino acids that exhibits at least 85% sequence identity to SEQ ID NO: 209, and the variable light chain set forth in SEQ ID NO: 286 or a sequence of amino acids that exhibits at least 85% sequence identity to SEQ ID NO: 286;

Modified anti-EGFR antibodies provided herein also include antibody fragments, which are derivatives of full-length antibody that contain less than the full sequence of the full-length antibodies but retain at least a portion of the specific binding abilities of the full-length antibody, for example the variable portions of the heavy and light chain. The antibody fragments also can include antigen-binding portions of an antibody that can be inserted into an antibody framework (e.g., chimeric antibodies) in order to retain the binding affinity of the parent antibody. Examples of antibody fragments include, but are not limited to, Fab, Fab′, F(ab′)₂, single-chain Fv (scFv), Fv, dsFv, diabody, Fd and Fd′ fragments, and other fragments, including modified fragments (see, for example, Methods in Molecular Biology, Vol. 207: Recombinant Antibodies for Cancer Therapy Methods and Protocols (2003); Chapter 1; p 3-25, Kipriyanov). Antibody fragments can include multiple chains linked together, such as by disulfide bridges and can be produced recombinantly. Antibody fragments also can contain synthetic linkers, such as peptide linkers, to link two or more domains. Methods for generating antigen-binding fragments are well-known known in the art and can be used to modify any antibody provided herein. Fragments of antibody molecules can be generated, such as for example, by enzymatic cleavage. For example, upon protease cleavage by papain, a dimer of the heavy chain constant regions, the Fc domain, is cleaved from the two Fab regions (i.e., the portions containing the variable regions). Alternatively, upon protease cleavage by pepsin can be used to prepare divalent F(ab′)₂ fragments of an antibody.

Single chain antibodies can be recombinantly engineered by joining a heavy chain variable region (V_{H}) and light chain variable region (V_{L}) of a specific antibody. The particular nucleic acid sequences for the variable regions can be cloned by standard molecular biology methods, such as, for example, by polymerase chain reaction (PCR) and other recombination nucleic acid technologies. Methods for producing scFvs are described, for example, by Whitlow and Filpula (1991) Methods, 2: 97-105; Bird et al. (1988) Science 242:423-426; Pack et al. (1993) Bio/Technology 11:1271-77; and U.S. Pat. Nos. 4,946,778, 5,840,300, 5,667,988, 5,658,727, 5,258,498).

Any of the above humanized anti-EGFR antibodies, or antigen-binding fragments, provided herein exhibit greater binding activity under conditions that include acidic pH of from 6.0 to 6.5, inclusive, and/or a lactate concentration of 15 mM to 20 mM, inclusive, compared to under conditions that include neutral pH of or about 7.4 and/or a lactate concentration of or about 1 mM, such that the ratio of binding activity is greater than 1.0, such as greater than 1.2, 1.3, 1.4, 1.5, 1.6, 1.7, 1.8, 1.9, 2.0, 3.0, 4.0, 5.0, 6.0, 7.0, 8.0, 9.0, 10.0, 20.0, 30.0, 40.0, 50.0 or more. Also, typically, any of the above humanized anti-EGFR antibodies, or antigen-binding fragments thereof, also effect significant productivity when produced in mammalian cells, particularly compared to the non-humanized parental antibody. For example, mammalian host cells containing nucleic acid encoding any of the above humanized anti-EGFR antibodies (e.g., those containing a nucleic acid encoding a heavy and light chain as set forth in Table 10) can effect expression of the antibody at a concentration that is greater than or greater than about or that is at least 1 mg/mL, 1.5 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 8.0 mg/mL, 9.0 mg/mL, 10.0 mg/mL or more.

3. Anti-EGFR Antibodies Containing 104D Modification

Also provided herein are modified anti-EGFR antibodies containing an amino acid replacement of aspartic acid (D), at a position corresponding to position 104 (designated 104D) of the variable domain of the heavy chain of an anti-EGFR antibody with reference to SEQ ID NO: 2 or 7. A position corresponding to position 104 in an unmodified anti-EGFR antibody can be determined by alignment of the variable heavy chain with the variable heavy chain set forth in SEQ ID NO: 2 or 7 (see, e.g., FIG. 2). Substitution of glutamic acid (E) at position 104 with aspartic acid (D) is a conservative mutation. Thus, any of the 104E antibodies described herein, can be conservatively mutated to generate a corresponding 104D anti-EGFR antibody.

The modified 104D anti-EGFR antibodies, or antigen-binding fragments thereof, provided herein, minimally contain a variable heavy chain and a variable light chain, or a portion thereof that is sufficient to bind EGFR antigen (e.g., human EGFR), or a soluble fragment thereof, when assembled into an antibody, whereby at least the variable heavy chain is modified by replacement with 104D. The resulting modified anti-EGFR antibodies can be full-length IgG1 antibodies, or can be fragments thereof, for example, a Fab, Fab′, F(ab′)₂, single-chain Fv (scFv), Fv, dsFv, diabody, Fd and Fd′ fragments. Further, the resulting modified anti-EGFR antibodies can contain a domain other than IgG1.

The 104D modification can be introduced into any anti-EGFR antibody described herein or known in the art, such as an unmodified anti-EGFR antibody (e.g., cetuximab antibody), antigen-binding fragment thereof or variant thereof. Exemplary unmodified anti-EGFR antibodies in which the amino acid replacement(s) herein can be made, include, but are not limited to, an anti-EGFR cetuximab antibody, or antigen-binding fragment or variant thereof, that contains a heavy chain set forth in any of SEQ ID NOS: 1, 2, 5, 6, 7, 12, 14 or 16, or an antigen-binding fragment or variant thereof containing at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity to any of SEQ ID NOS: 1, 2, 5, 6, 7, 12, 14 or 16. For example, an unmodified anti-EGFR antibody can contain a sequence of amino acids including a variable heavy chain (VH) set forth in SEQ ID NO: 2 and variable light chain (VL) set forth in SEQ ID NO: 4, a VH set forth in SEQ ID NO: 7 and a VL set forth in SEQ ID NO: 9, a VH set forth in SEQ ID NO: 7 and a VL set forth in SEQ ID NO: 11, a VH set forth in SEQ ID NO: 14 or a VL set forth in SEQ ID NO: 15, or a VH set forth in SEQ ID NO: 16 or a VL set forth in SEQ ID NO: 17, or variant thereof that contains a variable heavy and/or variable light chain that exhibits least 75%, 80%, 81%, 82%, 83%, 84%, 85%, 86%, 86%, 88%, 89%, 90%, 91%, 92%, 93%, 94%, 95%, 96%, 97%, 98%, 99% sequence identity to one or both of the variable heavy or light chains SEQ ID NOS.

The unmodified anti-EGFR antibody can be a full-length antibody or antigen-binding fragment thereof. For example, the unmodified anti-EGFR antibody can contain any of the VH or VL regions above and a constant region of the heavy and light chain including a heavy chain set forth in SEQ ID NO: 1 and a light chain set forth in SEQ ID NO: 3, a heavy chain set forth in SEQ ID NO: 5 and a light chain set forth in SEQ ID NO: 3, a heavy chain set forth in SEQ ID NO: 12 and a light chain set forth in SEQ ID NO: 13, a heavy chain set forth in SEQ ID NO: 6 and a light chain set forth in SEQ ID NO: 8 or a heavy chain set forth in SEQ ID NO: 6 and a light chain set forth in SEQ ID NO: 10, or can be an antigen-binding fragment of the full-length antibody or variant thereof that contains a heavy and/or light chain that exhibits least 75%, 80%, 81%, 82%, 83%, 84%, 85%, 86%, 86%, 88%, 89%, 90%, 91%, 92%, 93%, 94%, 95%, 96%, 97%, 98%, 99% to one or both of the heavy or light chains SEQ ID NOS. In any of such examples, modified anti-EGFR antibodies or antigen-binding fragments thereof provided herein can contain a variable heavy chain with the amino acid replacement Y104D, where the tyrosine (Y) at a position corresponding to position 104 is replaced with D.

The modified anti-EGFR antibody provided herein can contain only an amino acid replacement 104D in the variable heavy chain compared to the unmodified anti-EGFR antibody or can contain amino acid replacements or modifications, in addition to 104D, in one or both of the heavy chain or light chain. For example, modified anti-EGFR antibodies provided herein can contain at least or 1, 2, 3, 4, 5, 6, 7, 8, 9, 10, 11, 12, 13, 14, 15, 16, 17, 18, 19, 20, or more modified positions compared to the anti-EGFR antibody not containing the modification. Exemplary additional modifications in the heavy chain or light chain include any set forth in Section C.1 above (e.g., Tables 6 and 8). It is understood that in all examples of the modified 104D anti-EGFR antibodies provided herein, the modified anti-EGFR antibody contains an amino acid replacement 104D compared to the unmodified anti-EGFR antibody, and exhibits greater binding activity under conditions that include one or both of acidic pH of from 6.0 to 6.5, inclusive, and/or a lactate concentration of 10 mM to 20 mM, inclusive, compared to under conditions that include one or both of neutral pH of or about 7.4 and/or a lactate concentration of or of about 1 mM.

The modified 104D anti-EGFR antibodies provided herein exhibit greater binding activity under conditions that include one or both of acidic pH of from 6.0 to 6.5, inclusive, and/or a lactate concentration of 10 mM to 20 mM, inclusive, compared to under conditions that include one or both of neutral pH of or about 7.4 and/or a lactate concentration of or about 1 mM. For example, the ratio of binding activity under conditions that include one or both of pH 6.0 to 6.5/or and 10 mM to 20 mM lactate versus binding activity under conditions that include one or both of or about pH 7.4 and/or about or 1 mM lactate can be at least or greater than 1.1, 1.2, 1.3, 1.4, 1.5, 1.6, 1.7, 1.8, 1.9, 2.0, 2.5, 3.0, 3.5, 4.0, 4.5, 5.0, 6.0, 7.0, 8.0, 9.0, 10.0, 11.0, 12.0, 13.0, 14.0, 15.0, 20.0, 25.0, 30.0, 35.0, 40.0, 45.0, 50.0 or more. The modified anti-EGFR antibodies provided herein can exhibit the altered binding activity in the presence of physiologic concentrations of protein (e.g., 25% serum). Hence, the 104D antibodies provided herein can exhibit tumor selective EGFR binding activity, whereby binding activity is greater under conditions that exist in a tumor microenvironment compared to conditions that exist in a non-tumor microenvironment.

Any antibody described herein can be modified to contain a 104D substitution, including combinatorial mutant antibodies provided herein and humanized antibodies provided herein. The heavy chain and light chain sequences of non-limiting exemplary 104D modified anti-EGFR antibodies are set forth in Table 11 below. Also provided herein are anti-EGFR antibodies that contain a heavy chain and/or a light chain that contains a sequence of amino acids that exhibits at least 75%, 80%, 85%, 86%, 87%, 88%, 89%, 90%, 91%, 92%, 93%, 94%, 95%, 96%, 97%, 98%, 99% or more sequence identity to any of the SEQ ID NOS of the heavy chain and/or light chain set forth in Table 11, as long as the resulting antibody exhibits greater binding activity under conditions that include one or both of acidic pH of from 6.0 to 6.5, inclusive, and/or a lactate concentration of 10 mM to 20 mM, inclusive, compared to under conditions that include one or both of neutral pH of or about 7.4 and/or a lactate concentration of or about 1 mM.

TABLE 11
Exemplary 104D Modified Anti-EGFR Antibodies

| | Heavy chain | Light chain |
|---|---|---|
| | (variable chain | (variable chain |
| | corresponding to | corresponding to |
| | amino acids 1-119 | amino acids 1-107 |
| | of SEQ ID NO) | of SEQ ID NO) |
| Y104D | 67 | 8 |
| Y104D/Q111P | 53 | 8 |
| S25C/Y104D | 352 | 8 |
| S53G/Y104D | 353 | 8 |
| S53G/Y104D/Q111P | 354 | 8 |
| S25V/Y104D | 355 | 8 |
| S25V/Y104D/Q111P | 356 | 8 |
| S25V/S53G/Y104D | 357 | 8 |
| S25V/S53G/Y104D/Q111P | 358 | 8 |
| F27G/Y104D | 367 | 8 |
| F27G/Y104D/Q111P | 368 | 8 |
| F27G/S53G/Y104D | 369 | 8 |
| F27G/S53G/Y104D/Q111P | 370 | 8 |
| T30F/Y104D | 359 | 8 |
| T30F/Y104D/Q111P | 360 | 8 |
| T30F/S53G/Y104D | 361 | 8 |
| T30F/S53G/Y104D/Q111P | 362 | 8 |
| D72L/Y104D | 363 | 8 |
| D72L/Y104D/Q111P | 364 | 8 |
| S53G/D72L/Y104D | 365 | 8 |
| S53G/D72L/Y104D/Q111P | 366 | 8 |
| Y104D/I29S | 67 | 124 |
| Y104D/Q111P/I29S | 53 | 124 |

Humanized Antibodies containing 104D

| | | |
|---|---|---|
| D-h | 57 | 181 |
| DP-h1 | 372 | 153 |
| DP-h2 | 372 | 160 |
| DP-h3 | 55 | 153 |
| DP-h4 | 372 | 167 |
| DP-h5 | 372 | 174 |
| DP-h6 | 372 | 181 |
| DP-h7 | 55 | 181 |
| DP-h8 | 372 | 188 |
| DP-h9 | 374 | 181 |
| DP-h10 | 376 | 195 |
| DP-h12 | 374 | 195 |
| DP-h13 | 376 | 202 |
| DP-h14 | 374 | 202 |
| FDP-h1 | 378 | 251 |
| FDP-h2 | 380 | 251 |
| FDP-h3 | 65 | 258 |
| FDP-h4 | 382 | 258 |
| FDP-h5 | 384 | 265 |
| FDP-h6 | 386 | 272 |
| FDP-h7 | 65 | 272 |
| FDP-h8 | 382 | 272 |
| FDP-h9 | 384 | 279 |
| FDP-h10 | 388 | 279 |
| FDP-h11 | 65 | 279 |
| FDP-h2 | 382 | 279 |
| FDP-h13 | 384 | 286 |
| FDP-h14 | 388 | 286 |
| FDP-h15 | 65 | 286 |
| FDP-h16 | 382 | 286 |
| FDP-h17 | 384 | 293 |
| FDP-h18 | 388 | 300 |
| FDP-h19 | 378 | 300 |
| FDP-h20 | 378 | 279 |
| FDP-h21 | 378 | 286 |

4. Conjugates

Also provided herein are conjugates that contain a modified anti-EGFR antibody provided herein linked directly or via a linker to one or more targeted agents. These conjugates contain the following components: antibody (Ab), (linker (L))_{q}, (targeted agent)ₘ and are represented by the formula: Ab-(L)_{q}-(targeted agent)ₘ, where q is 0 or more and m is at least 1. Thus, the conjugates provided herein contain one or more targeted agents covalently linked to an modified antibody provided herein. The conjugates exhibit greater binding activity for an EGFR antigen (e.g., human EGFR) or soluble fragment thereof under conditions that include one or both of acidic pH of from 6.0 to 6.5, inclusive, and/or a lactate concentration of 10 mM to 20 mM, inclusive, compared to under conditions that include one or both of neutral pH of or about 7.4 and/or a lactate concentration of or about 1 mM.

Hence, these conjugates, also called antibody-drug conjugates (ADC) or immunoconjugates, can be used for targeted delivery of cytotoxic or cytostatic agents, i.e., drugs to kill or inhibit tumor cells expressing EGFR in the treatment of cancer. Such conjugates exhibit selectivity to tumor cells that are desired to be eliminated over non-diseased cells, and thereby do not result in unacceptable levels of toxicity to normal cells. Therefore, the conjugates achieve maximal efficacy with minimal toxicity and reduced side effects. Hence, such compounds can be used in the methods described herein of diagnosis or treatment of cancer and other diseases or disorders.

As stated above, the number of targeted agents is designated by the variable m, where m is an integer of 1 or greater. The targeted agent is conjugated to an antibody provided herein by the number of linkers designated by the variable q, where q is 0 or any integer. The variables q and m are selected such that the resulting conjugate interacts with the EGFR of target cells, in particular, tumor cells, and the targeted agent is internalized by the target cell. Typically, m is between 1 and 8. q is 0 or more, depending upon the number of linked targeting and targeted agents and/or functions of the linker; q is generally 0 to 4. When more than one targeted agent is present in a conjugate, the targeted agents may be the same or different.

The targeted agents can be covalently linked to the modified anti-EGFR antibody directly or by one or more linkers. Any suitable association among the elements of the conjugate is contemplated as long as the resulting conjugates interact with the EGFR of a target cell such that internalization of the associated targeted agent is effected. Thus, the conjugates provided herein can be produced as fusion proteins, can be chemically coupled, or can include a fusion protein portion and a chemically linked portion or any combination thereof

The targeted agents also can be modified to render them more suitable for conjugation with the linker and/or the modified anti-EGFR antibody or to increase their intracellular activity. For example, in the case of polypeptide targeted agents, such modifications include, but are not limited to, the introduction of a Cys residue at or near the N-terminus or C-terminus, derivatization to introduce reactive groups, such as thiol groups, and/or addition of sorting signals, such as (Xaa-Asp-Glu-Leu)ₙ (SEQ ID NO. 350 not in sequence listing) where Xaa is Lys or Arg, preferably Lys, and n is 1 to 6, preferably 1-3, at, preferably, the carboxy-terminus of the targeted agent (see, e.g., Seetharam et al. (1991) J. Biol. Chem. 266:17376-17381; and Buchner et al. (1992) Anal. Biochem. 205:263-270), that direct the targeted agent to the endoplasmic reticulum.

In other examples, the targeted agent can be modified to eliminate one or more cysteine residues, for example, to provide more predictable thiol conjugation at preferred locations. Care must be taken to avoid altering specificity of the resulting modified targeted agent, unless such alteration is desired. In all instances, particular modifications can be determined empirically.

The linker, L, attaches the antibody to the targeted agent through covalent bond(s). The linker can be a peptide or a non-peptide and can be selected to relieve or decrease steric hindrance caused by proximity of the targeted agent to the modified anti-EGFR antibody and/or to increase or alter other properties of the conjugate, such as the specificity, toxicity, solubility, serum stability and/or intracellular availability of the targeted moiety and/or to increase the flexibility of the linkage between the anti-EGFR antibody and the targeted agent.

When fusion proteins are contemplated, the linker is selected such that the resulting nucleic acid molecule encodes a fusion protein that binds to and is internalized by cells in a tumor microenvironment that express EGFR and all or a portion of the internalized protein preferably traffics to the cytoplasm. It also is contemplated that several linkers can be joined in order to employ the advantageous properties of each linker. In such instances, the linker portion of a conjugate may contain more than 50 amino acid residues. The number of residues is not important as long as the resulting fusion protein binds to EGFR of the target cell and internalizes the linked targeted agent via a pathway that traffics the targeted agent to the cytoplasm and/or nucleus.

The targeted agent can be a protein, peptide, nucleic acid, small molecule, therapeutic moiety, or other agent in which targeted delivery to a selected population of tumor cells is desired. Such targeted agents include, but are not limited to, cytotoxic agents, DNA and RNA nucleases, toxins, drugs or other agents. Therapeutic moieties include, but are not limited to, cytotoxic moieties, radioisotopes, chemotherapeutic agents, lytic peptides and cytokines. Exemplary therapeutic moieties include, but are not limited to, taxol; cytochalasin B; gramicidin D; ethidium bromide; emetine; mitomycin; etoposide; teniposide; vincristine; vinblastine; colchicine; doxorubicin; daunorubicin; dihydroxy anthracin dione; maytansine or an analog or derivative thereof; an auristatin or a functional peptide analog or derivative thereof; dolastatin 10 or 15 or an analog thereof; irinotecan or an analog thereof; mitoxantrone; mithramycin; actinomycin D; 1-dehydrotestosterone; a glucocorticoid; procaine; tetracaine; lidocaine; propranolol; puromycin; calicheamicin or an analog or derivative thereof; an antimetabolite; an alkylating agent; a platinum derivative; duocarmycin A, duocarmycin SA, rachelmycin (CC-1065), or an analog or derivative thereof; an antibiotic; a pyrrolo[2,1-c][1,4]-benzodiazepine (PDB); a toxin; ribonuclease (RNase); DNase I, Staphylococcal enterotoxin A; and pokeweed antiviral protein.

Drugs also can be used as a targeted agent in these methods. Such drugs include 5-fluorouracil, vinca alkaloids, and antibiotics such as dactinomycin, bleomycin, daunorubicin, doxorubicin, idarubicin, methotrexate, mithramycin, mitomycin, mitoxantrone, plicamycin and anthramycin (AMC), neocarzinostatin and vindesine.

Toxins used in antibody-toxin conjugates include bacterial toxins such as diphtheria toxin, and active fragments thereof and hybrid molecules, plant toxins, such as ricin toxin, small molecule toxins such as geldanamycin, maytansinoids, such as DM1, DM3 and DM4, and calicheamicin. Finally, the auristatin peptides, auristatin E (AE), monomethylauristatin E (MMAE), and monomethylauristatin F (MMAF), synthetic analogs of dolastatin can be employed. Other toxins include cholera toxin, a Shiga-like toxin, LT toxin, C3 toxin, Shiga toxin, pertussis toxin, tetanus toxin, soybean Bowman-Birk protease inhibitor, Pseudomonas exotoxin, alorin, saporin, modeccin, galanin, abrin A chain, modeccin A chain, alpha-sarcin, Aleurites fordii proteins, dianthin proteins, Phytolacca americana proteins, momordica charantia inhibitor, curcin, crotin, gelonin, mitogillin, restrictocin, phenomycin, and enomycin toxins. The toxins can effect their cytotoxic and cytostatic activity by mechanisms including tubulin binding, DNA binding, or topoisomerase inhibition.

a. Targeted Agents

The targeted agent can be a protein, peptide, nucleic acid, small molecule, therapeutic moiety, or other agent in which targeted delivery to a selected population of tumor cells is desired. Such targeted agents include, but are not limited to, cytotoxic agents, DNA and RNA nucleases, toxins, drugs or other agents.

### i. Maytansinoid Drug Moieties

A cytotoxic moiety as a targeted agent in the conjugates include Maytansinoid drug moieties, including those described in U.S. Pat. No. 8,142,784. Maytansine compounds inhibit cell proliferation by inhibiting the formation of microtubules during mitosis through inhibition of polymerization of the microtubule protein, tubulin (Remillard et al. (1975) Science 189:1002-1005; U.S. Pat. No. 5,208,020). Maytansine and maytansinoids are highly cytotoxic but their clinical use in cancer therapy has been greatly limited by their severe systemic side-effects primarily attributed to their poor selectivity for tumors. Clinical trials with maytansine had been discontinued due to serious adverse effects on the central nervous system and gastrointestinal system (Issell et al. (1978) Can. Treatment. Rev. 5:199-207).

Maytansinoid drug moieties are attractive drug moieties in antibody-drug conjugates because they are: (i) relatively accessible to prepare by fermentation or chemical modification, derivatization of fermentation products, (ii) amenable to derivatization with functional groups suitable for conjugation through the non-disulfide linkers to antibodies, (iii) stable in plasma, and (iv) effective against a variety of tumor cell lines.

Maytansine compounds suitable for use as maytansinoid drug moieties are well-known in the art, and can be isolated from natural sources according to known methods, produced using genetic engineering techniques (see Yu et al. (2002) PNAS 99:7968-7973), or maytansinol and maytansinol analogs prepared synthetically according to known methods.

Exemplary maytansinoid drug moieties include those having a modified aromatic ring, such as: C-19-dechloro (U.S. Pat. No. 4,256,746) (prepared by lithium aluminum hydride reduction of ansamitocin P2); C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/−dechloro (U.S. Pat. No. 4,294,757) (prepared by acylation using acyl chlorides); and those having modifications at other positions.

Exemplary maytansinoid drug moieties also include those having modifications such as: C-9-SH, prepared by the reaction of maytansinol with H₂S or P₂S₅ (U.S. Pat. No. 4,424,219); C-14-alkoxymethyl(demethoxy/CH₂OR)(U.S. Pat. No. 4,331,598); C-14-hydroxymethyl or acyloxymethyl (CH₂OH or CH₂OAc) prepared from Nocardia (U.S. Pat. No. 4,450,254); C-15-hydroxy/acyloxy, prepared by the conversion of maytansinol by Streptomyces (U.S. Pat. No. 4,364,866); C-15-methoxy, isolated from Trewia nudiflora (U.S. Pat. No. 4,313,946 and U.S. Pat. No. 4,315,929); C-18-N-demethyl, prepared by the demethylation of maytansinol by Streptomyces (U.S. Pat. No. 4,362,663 and U.S. Pat. No. 4,322,348); and 4,5-deoxy, prepared by the titanium trichloride/LAH reduction of maytansinol (U.S. Pat. No. 4,371,533).

Many positions on maytansine compounds are known to be useful as the linkage position, depending upon the type of link. For example, for forming an ester linkage, the C-3 position having a hydroxyl group, the C-14 position modified with hydroxymethyl, the C-15 position modified with a hydroxyl group and the C-20 position having a hydroxyl group are all suitable.

Maytansinoid drug moieties can be linked to a modified anti-EGFR antibody by direct conjugation or using any of the linkers provided herein. In particular examples, the cytotoxic or drug agent is mertansine, also known as DM1 (N₂′-deacetyl-N₂′-(3-mercapto-1-oxopropyl)-maytansine). Mertansine can be linked via 4-mercaptovaleric acid. An emtansine conjugate also can be formed with the antibodies herein using the linker 4-(3-mercapto-2,5-dioxo-1-pyrrolidinylmethyl)-cylohexanecarboxylic acid (MCC).

### ii. Auristatins and Dolastatins Drug Moieties

A cytotoxic moiety as a targeted agent in the conjugates include auristatins and dolastatins, including those described in U.S. Publication No. US2011/0217321. Dolastatins and auristatins have been shown to interfere with microtubule dynamics, GTP hydrolysis, and nuclear and cellular division (Woyke et al. (2001) Antimicrob. Agents and Chemother. 45(12):3580-3584) and have anticancer (U.S. Pat. No. 5,663,149) and antifungal activity (Pettit et al. (1998) Antimicrob. Agents Chemother. 42:2961-2965). Further, auristatins are highly potent, synthetic, stable, and amenable to chemical modification to allow for linker attachment (Senter (2009) Curr Opin Chem Biol 13:235-244).

Because auristatins are synthetic, integral structural modifications can be made to significantly alter the properties of the parent drug. For example, monomethylauristatin F (MMAF) terminates with the amino acid residue phenylalanine, which impairs cell membrane permeability (Doronina et al., (2006) Bioconjug Chem. 17:114-124). Thus, conjugation of MMAF to an ADC can facilitate selective drug uptake by antigen-positive cells (Doronina et al., (2006) Bioconjug Chem. 17:114-124; Doronina et al., (2003) Nat Biotechnol. 21:778-784).

The dolastatin or auristatin drug moiety can be attached to antibodies through the N (amino) terminus or the C (carboxyl) terminus of the peptidic drug moiety (WO 2002/088172). Exemplary auristatin embodiments include N-terminally and C-terminally linked monomethylauristatin drug moieties MMAE and MMAF (Senter et al. (2004) “Proceedings of the American Association for Cancer Research,” Volume 45, Abstract Number 623, and presented Mar. 28, 2004; U.S. Publication No. 2011/0020343).

Dolastatin or auristatin can be linked to a modified anti-EGFR antibody by direct conjugation or using any of the linkers provided herein. In particular examples, dolastatin or auristatin can be linked to an anti-EGFR antibody with a peptide linker, such as valine-citrulline (Val-Cit).

### iii. Pyrrolobenzodiazepines (PBDs)

A cytotoxic moiety as a targeted agent in the conjugates include pyrrolobenzodiazepines (PBDs) (or pyrrolo[2,1-c][1, 4]-benzodiazepines), which are sequence-selective DNA alkylating antibiotics with significant antitumor properties. PBDs have the ability to recognize and bond specific sequences of DNA; the preferred DNA sequence is PuGPu (Purine-Guanine-Purine). PBDs also can bond to PuGPy (Purine-Guanine-Pyrimidine) or PyGPu sequences, preferably over PyGPy sequences.

PBDs can be naturally occurring or synthetic. Naturally occurring PBDs include abbeymycin (Hochlowski, et al., J. Antibiotics, 40, 145-148 (1987)), anthramycin (Leimgruber, et al., J. Am. Chem. Soc, 87, 5793-5795 (1965); Leimgruber, et al., J. Am. Chem. Soc, 87, 5791-5793 (1965)), chicamycin (Konishi, et al., J. Antibiotics, 37, 200-206 (1984)), DC-81 (Thurston, et al., Chem. Brit, 26, 767-772 (1990); Bose, et al., Tetrahedron, 48, 751-758 (1992)), mazethramycin (Kuminoto, et al., J. Antibiotics, 33, 665-667 (1980)), neothramycins A and B (Takeuchi, et al., J. Antibiotics, 29, 93-96 (1976)), porothramycin (Tsunakawa, et al., J. Antibiotics, 41, 1366-1373 (1988)), prothracarcin (Shimizu, et al, J. Antibiotics, 29, 2492-2503 (1982); Langley and Thurston, J. Org. Chem., 52, 91-97 (1987)), sibanomicin (DC-102)(Hara, et al., J. Antibiotics, 41, 702-704 (1988); Itoh, et al., J. Antibiotics, 41, 1281-1284 (1988)), sibiromycin (Leber, et al., J. Am. Chem. Soc, 110, 2992-2993 (1988)), and tomamycin (Arima, et al., J. Antibiotics, 25, 437-444 (1972)). Synthesis of PBDs and generation of synthetic analogs also have been described (see, e.g., U.S. Pat. Nos. 6,562,806, 6,608,192 6,747,144, and 7,049,311, 7,528,126).

PBDs are of the general structure: (Formula 1)

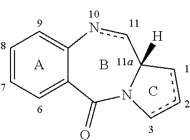

PBDs differ in the number, type and position of substituents, in both their aromatic A rings and pyrrolo C rings, and in the degree of saturation of the C ring. In the B-ring there is either an imine (N═C), a carbinolamine (NH—CH(OH)), or a carbinolamine methyl ether (NH—CH(OMe)) at the N10-C11 position which is the electrophilic center responsible for alkylating DNA. All of the known natural products have an (S)-configuration at the chiral C11 a position which provides them with a right-handed twist when viewed from the C ring towards the A ring. This gives them the appropriate three-dimensional shape for isohelicity with the minor groove of B-form DNA (Kohn, In Antibiotics III. Springer-Verlag, New York, pp. 3-11 (1975); Hurley and Needham-VanDevanter, Acc. Chem. Res., 19, 230-237 (1986)). PBDs form a covalent, aminal linkage with the exocyclic N2 of the guanine in the PuGPu consensus sequence, forming a PBD/DNA adduct which interferes with DNA processing and leads to cell cycle arrest and apoptosis. Thus PBDs are effective antitumor agents.

Dimers of PBDs also are effective antitumor agents. PBD dimers cover six base pairs instead of three base pairs covered by the PBD monomer. Further, the PBDs in the dimer can bond sequences in the complementary strands of DNA (i.e., an interstrand guanine-guanine cross-link), leading to sequence-selective DNA cross-linking. PBD dimer-induced cross-linking prevents strand separation, thereby preventing DNA replication. This results in cell cycle arrest and apoptosis in the G2/M interface. The increased coverage of PBD dimers, compared to PBD monomers, in addition to DNA cross-linking leads to substantially increased efficacy as anticancer agents.

PBD dimers can be homodimers or heterodimers, and are synthesized by joining the two monomer PBD units together through their C8 positions via a flexible linker. Commonly used linkers include propyldioxy (PBD-C8-O—(CH2)3-O-C8′-PBD) and pentyldioxy (PBD-C8—O—(CH2)5-O-C8′-PBD′). The properties of the linker, such as the length of the linker, can be selected to target the dimer to specific DNA sequences (Rahman et al., (2011) Nucleic Acids Res. 39(13): 5800-5812 and Gregson et al., (2004) J Med Chem 47:1161-1174). Exemplary inter-PBD linkers are described in Bose et al., (1992) J Am Chem Soc. 114:4939-4941, Bose et al., (1992) J Chem Soc Chem Commun. 14:1518-1520, Thurston et al., (1996) J Org Chem. 61:8141-8147, Gregson et al., (2001) J Med Chem. 44:737-738, and Gregson et al., J Med Chem 2004; 47:1161-1174. Exemplary PBD dimers have been described in the art (see, e.g., U.S. Pat. Nos. 6,562,806, 6,608,192 6,747,144, 7,049,311, 7,528,126, 7,741,319, 8,592,576) and include, but are not limited to, compounds designated DSB-120 (U.S. Pat. No. 7,049,311), DRH-165 (U.S. Pat. No. 7,049,311), ELB21 (Rahman et al., (2011) Nucleic Acids Res. 39(13): 5800-5812), SG2000/SJG136 (Rahman et al., (2011) Nucleic Acids Res. 39(13): 5800-5812; U.S. Pat. No. 7,049,311), SG2057/DRG16 (Rahman et al., (2011) Nucleic Acids Res. 39(13): 5800-5812), SG2202 (U.S. Pat. No. 7,741,319; Hartley et al., (2010) Cancer Res. 70(17):6849-6858), SG2285 (Hartley et al., (2010) Cancer Res. 70(17):6849-6858), SG3132 (US 20130028919).

PBDs and PBD dimers can be conjugated to any of the antibodies provided herein by any method, including, but not limited to thiol, amine and phenol conjugation. Typically, the PBD or PBD dimer is conjugated to the antibody using a cleavable linker, that is stable in in vivo circulation, such that the PBD or PBD dimer is released from the antibody following cleavage of the linker inside the target cell. In some examples, PBD or PBD dimer can be conjugated to inter-chain cysteines. In some examples, the antibody can be modified to replace amino acid(s) to insert or remove an inter-chain cysteine to facilitate directed thiol linkage of the PBD or PBD dimer

### iv. Cell Toxin Moieties

Cell toxins suitable for use in the methods and compositions include small molecules, such as DNA cleaving agents, and proteinaceous cell toxins, including, but are not limited to, bacterial, fungal, plant, insect, snake and spider toxins. Exemplary cell toxins contemplated for incorporation in the conjugates provided herein are set forth in Table 12.

TABLE 12
Exemplary Amino Acid Sequences of Toxins

| Toxin | SEQ ID NO |
|---|---|
| Bryodin | 389 |
| Saporin-6 | 390 |
| Anti-Viral Protein MAP | 391 |
| Shiga Toxin A-Chain | 392 |
| Shiga-Like Toxin Subunit A (Verotoxin 2) | 393 |
| Trichosanthin | 394 |

### (a) DNA Cleaving Agents

Examples of DNA cleaving agents suitable for inclusion as the cell toxin in the chimeric ligand-toxin used in practicing the methods include, but are not limited to, anthraquinone-oligopyrrol-carboxamide, benzimidazole, leinamycin; dynemycin A; enediyne; as well as biologically active analogs or derivatives thereof (i.e., those having a substantially equivalent biological activity). Known analogs and derivatives are disclosed, for examples in Islam et al., J. Med. Chem. 34 2954-61, 1991; Skibo et al., J. Med. Chem. 37:78-92, 1994; Behroozi et al., Biochemistry 35:1768-74, 1996; Helissey et al., Anticancer Drug Des. 11:527-51, 1996; Unno et al., Chem. Pharm. Bull. 45:125-33, 1997; Unno et al., Bioorg. Med. Chem., 5:903-19, 1997; Unno et al., Bioorg. Med. Chem., 5: 883-901, 1997; and Xu et al., Biochemistry 37:1890-7, 1998). Other examples include, but are not limited to, endiyne quinone imines (U.S. Pat. No. 5,622,958); 2,2r-bis(2-aminoethyl)-4-4′-bithiazole (Lee et al., Biochem. Mol. Biol. Int. 40:151-7, 1996); ellipticine-salen.copper conjugates (Routier et al., Bioconjug. Chem., 8: 789-92, 1997).

### (b) Antimetabolites

Examples of antimetabolites useful for inclusion as the cell toxin in the chimeric ligand-toxin include, but are not limited to, 5-fluorouracil, methotrexate, melphalan, daunomycin, doxorubicin, nitrogen mustard and mitomycin c.

### (c) Proteinaceous Cell Toxins

Examples of proteinaceous cell toxins useful for incorporation into the chimeric ligand-toxins used in the methods include, but are not limited to, type one and type two ribosome inactivating proteins (RIP). Useful type one plant RIPs include, but are not limited to, dianthin 30, dianthin 32, lychnin, saporins 1-9, pokeweed activated protein (PAP), PAP II, PAP-R, PAP-S, PAP-C, mapalmin, dodecandrin, bryodin-L, bryodin, Colicin 1 and 2, luffin-A, luffin-B, luffin-S, 19K-protein synthesis inhibitory protein (PSI), 15K-PSI, 9K-PSI, alpha-kirilowin, beta-kirilowin, gelonin, momordin, momordin-II, momordin-Ic, MAP-30, alpha-momorcharin, beta-momorcharin, trichosanthin, TAP-29, trichokirin; barley RIP; flax RIP, tritin, corn RIP, Asparin 1 and 2. Useful type two RIPs include, but are not limited to, volkensin, ricin, nigrin-b, CIP-29, abrin, modeccin, ebulitin-α, ebulitin-β, ebulitin-γ, vircumin, porrectin, as well as the biologically active enzymatic subunits thereof (Stirpe et al., Bio/Technology 10:405-12, 1992; Pastan et al., Annu. Rev. Biochem. 61:331-54; Brinkmann and Pastan, Biochim. et Biophys. Acta 1198:27-45, 1994; and Sandvig and Van Deurs, Physiol. Rev. 76:949-66, 1996).

### (d) Bacterial Toxins

Examples of bacterial toxins useful as cell toxins include, but are not limited to, shiga toxin and shiga-like toxins (i.e., toxins that have the same activity or structure), as well as the catalytic subunits and biologically functional fragments thereof. These bacterial toxins also are type two RIPs (Sandvig and Van Deurs, Physiol. Rev. 76:949-66, 1996; Armstrong, J. Infect. Dis., 171:1042-5, 1995; Kim et al., Microbiol. Immunol. 41:805-8, 1997, and Skinner et al., Microb. Pathog. 24:117-22, 1998). Additional examples of useful bacterial toxins include, but are not limited to, Pseudomonas exotoxin and Diphtheria toxin (Pastan et al., Annu. Rev. Biochem. 61:331-54; and Brinkmann and Pastan, Biochim. et Biophys. Acta 1198:27-45, 1994). Truncated forms and mutants of the toxin enzymatic subunits also can be used as a cell toxin moiety (Pastan et al., Annu. Rev. Biochem. 61:331-54; Brinkmann and Pastan, Biochim. et Biophys. Acta 1198:27-45, 1994; Mesri et al., J. Biol. Chem. 268:4853-62, 1993; Skinner et al., Microb. Pathog. 24:117-22, 1998; and U.S. Pat. No. 5,082,927). Other targeted agents include, but are not limited to the more than 34 described Colicin family of RNase toxins which include colicins A, B, D, E1-9, cloacin DF13 and the fungal RNase, α-sarcin (Ogawa et al. Science 283: 2097-100, 1999; Smarda et al., Folia Microbiol (Praha) 43:563-82, 1998; Wool et al., Trends Biochem. Sci., 17: 266-69, 1992).

### (e) Porphyrins and Other Light Activated Toxins

Porphyrins are well-known light activatable toxins that can be readily cross-linked to proteins (see, e.g., U.S. Pat. Nos. 5,257,970; 5,252,720; 5,238,940; 5,192,788; 5,171,749; 5,149,708; 5,202,317; 5,217,966; 5,053,423; 5,109,016; 5,087,636; 5,028,594; 5,093,349; 4,968,715; 4,920,143 and International Publication No. WO 93/02192).

### v. Nucleic Acids for Targeted Delivery

The conjugates provided herein also can be used to deliver nucleic acids to targeted cells. The nucleic acids include DNA intended to modify the genome of a cell and thereby effect genetic therapy, and DNA and RNA for use as antisense agents. The nucleic acids include antisense RNA, DNA, ribozymes and other oligonucleotides that are intended to be used as antisense agents. The nucleic acids can also include RNA trafficking signals, such as viral packaging sequences (see, e.g., Sullenger et al. (1994) Science 262:1566-1569). The nucleic acids also include DNA molecules that encode intact genes or that encode proteins intended to be used in gene therapy.

DNA (or RNA) that may be delivered to a cell to effect genetic therapy includes DNA that encodes tumor-specific cytotoxic molecules, such as tumor necrosis factor, viral antigens and other proteins to render a cell susceptible to anti-cancer agents, and DNA encoding genes, such as the defective gene (CFTR) associated with cystic fibrosis (see, e.g., International Application WO 93/03709; and Riordan et al. (1989) Science 245:1066-1073), to replace defective genes.

Nucleic acids and oligonucleotides for use as described herein can be synthesized by any method known to those of skill in the art (see, e.g., WO 93/01286 and U.S. Pat. Nos. 5,218,088; 5,175,269; and 5,109,124). Identification of oligonucleotides and ribozymes for use as antisense agents is well within the skill in the art. Selection of DNA encoding genes for targeted delivery for genetic therapy also is well within the level of skill of those in the art. For example, the desirable properties, lengths and other characteristics of such oligonucleotides are well-known. Antisense oligonucleotides are designed to resist degradation by endogenous nucleolytic enzymes and include, but are not limited to: phosphorothioate, methylphosphonate, sulfone, sulfate, ketyl, phosphorodithioate, phosphoramidate, phosphate esters, and other such linkages (see, e.g., Agrawal et al. (1987) Tetrahedron Lett. 28:3539-3542; Miller et al. (1971) J. Am. Chem. Soc. 93:6657-6665; Stec et al. (1985) Tetrahedron Lett. 26:2191-2194; Moody et al. (1989) Nucl. Acids Res. 17:4769-4782; Letsinger et al. (1984) Tetrahedron 40:137-143; Eckstein (1985) Annu. Rev. Biochem. 54:367-402; Eckstein (1989) Trends Biochem. Sci. 14:97-100; Stein (1989) In: Oligodeoxynucleotides. Antisense Inhibitors of Gene Expression, Cohen, ed, Macmillan Press, London, pp. 97-117; Jager et al. (1988) Biochemistry 27:7237-7246).

### (a) Antisense Nucleotides, Including: Antisense Oligonucleotides; Triplex Molecules; Dumbbell Oligonucleotides; DNA; Extracellular Protein Binding Oligonucleotides; and Small Nucleotide Molecules

Antisense nucleotides are oligonucleotides that specifically bind to mRNA that has complementary sequences, thereby preventing translation of the mRNA (see, e.g., U.S. Pat. No. 5,168,053 to Altman et al. U.S. Pat. No. 5,190,931 to Inouye, U.S. Pat. No. 5,135,917 to Burch; U.S. Pat. No. 5,087,617 to Smith and Clusel et al. (1993) Nucl. Acids Res. 21:3405-3411, which describes dumbbell antisense oligonucleotides). Triplex molecules refer to single DNA strands that target duplex DNA and thereby prevent transcription (see, e.g., U.S. Pat. No. 5,176,996, which describes methods for making synthetic oligonucleotides that bind to target sites on duplex DNA).

### (b) Ribozymes

Ribozymes are RNA constructs that specifically cleave messenger RNA. There are at least five classes of ribozymes that are known that are involved in the cleavage and/or ligation of RNA chains. Ribozymes can be targeted to any RNA transcript and can catalytically cleave such transcript (see, e.g., U.S. Pat. Nos. 5,272,262; 5,144,019 5,168,053; 5,180,818; 5,116,742 and 5,093,246, which describe ribozymes and methods for production thereof). Any such ribosome may be linked to a conditionally active anti-EGFR antibody for delivery to EGFR bearing cells under acidic conditions.

The ribozymes may be delivered to the targeted cells as DNA encoding the ribozyme linked to a eukaryotic promoter, such as a eukaryotic viral promoter, generally a late promoter, such that upon introduction into the nucleus, the ribozyme will be directly transcribed. In such instances, the construct will also include a nuclear translocation sequence, generally as part of the targeting agent or as part of a linker in order to render it suitable for delivering linked nucleic acids to the nucleus.

### (c) Nucleic Acids Encoding Therapeutic Products for Targeted Delivery

Among the DNA that encodes therapeutic products contemplated for use is DNA encoding correct copies anticancer agents, such as tumor necrosis factors, and cytotoxic agents, such as shiga A1 toxin or saporin to EGFR bearing tumor cells. The conjugate should include a nuclear translocation sequence (NTS). If the conjugate is designed such that the targeting agent and linked DNA is cleaved in the cytoplasm, then the NTS should be included in a portion of the linker that remains bound to the DNA, so that, upon internalization, the conjugate will be trafficked to the nucleus. The nuclear translocation sequence (NTS) may be a heterologous sequence or a may be derived from the selected chemokine receptor targeting agent. A typical consensus NTS sequence contains an amino-terminal proline or glycine followed by at least three basic residues in an array of seven to nine amino acids (see, e.g., Dang et al. (1989) J. Biol. Chem. 264:18019-18023).

### (d) Coupling of Nucleic Acids to Proteins

To effect chemical conjugation herein, the targeting agent is linked to the nucleic acid either directly or via one or more linkers. Methods for conjugating nucleic acids, at the 5′ ends, 3′ ends and elsewhere, to the amino and carboxyl termini and other sites in proteins are known to those of skill in the art (for a review see e.g., Goodchild, (1993) In: Perspectives in Bioconjugate Chemistry, Mears, Ed., American Chemical Society, Washington, D.C. pp. 77-99). For example, proteins have been linked to nucleic acids using ultraviolet irradiation (Sperling et al. (1978) Nucleic Acids Res. 5:2755-2773; Fiser et al. (1975) FEBS Lett. 52:281-283), bifunctional chemicals (Bäumert et al. (1978) Eur. J. Biochem. 89:353-359; and Oste et al. (1979) Mol. Gen. Genet. 168:81-86), and photochemical cross-linking (Vanin et al. (1981) FEBS Lett. 124:89-92; Rinke et al. (1980) J. Mol. Biol. 137:301-304; Millon et al. (1980) Eur. J. Biochem. 110:485-492).

In particular, the reagents (N-acetyl-N′-(p-glyoxylylbenzolyl)cystamine and 2-iminothiolane have been used to couple DNA to proteins, such as α₂-macroglobulin (α₂M) via mixed disulfide formation (see, Cheng et al. (1983) Nucleic Acids Res. 11:659-669). N-acetyl-N′-(p-glyoxylylbenzolyl)cystamine reacts specifically with non-paired guanine residues and, upon reduction, generates a free sulfhydryl group. 2-Iminothiolane reacts with proteins to generate sulfhydryl groups that are then conjugated to the derivatized DNA by an intermolecular disulfide interchange reaction. Any linkage may be used provided that, upon internalization of the conjugate the targeted nucleic acid is active. Thus, it is expected that cleavage of the linkage may be necessary, although it is contemplated that for some reagents, such as DNA encoding ribozymes linked to promoters or DNA encoding therapeutic agents for delivery to the nucleus, such cleavage may not be necessary.

Thiol linkages readily can be formed using heterobifunctional reagents Amines have also been attached to the terminal 5′ phosphate of unprotected oligonucleotides or nucleic acids in aqueous solutions by reacting the nucleic acid with a water-soluble carbodiimide, such as 1-ethyl-3′[3-dimethylaminopropyl]carbodiimide (EDC) or N-ethyl-N′(3-dimethylaminopropylcarbodiimidehydrochloride (EDCI), in imidazole buffer at pH 6 to produce the 5′ phosphorimidazolide. Contacting the 5′ phosphorimidazolide with amine-containing molecules and ethylenediamine, results in stable phosphoramidates (see, e.g., Chu et al. (1983) Nucleic Acids Res. 11:6513-6529; and WO 88/05077). In particular, a solution of DNA is saturated with EDC, at pH 6 and incubated with agitation at 4° C. overnight. The resulting solution is then buffered to pH 8.5 by adding, for example about 3 volumes of 100 mM citrate buffer, and adding about 5 μg-about 20 μg of a chemokine receptor targeting agent, and agitating the resulting mixture at 4° C. for about 48 hours. The unreacted protein may be removed from the mixture by column chromatography using, for example, SEPHADEX G75 (Pharmacia) using 0.1 M ammonium carbonate solution, pH 7.0 as an eluting buffer. The isolated conjugate may be lyophilized and stored until used.

U.S. Pat. No. 5,237,016 provides methods for preparing nucleotides that are bromacetylated at their 5′ termini and reacting the resulting oligonucleotides with thiol groups. Oligonucleotides derivatized at their 5′-termini bromoacetyl groups can be prepared by reacting 5′-aminohexyl-phosphoramidate oligonucleotides with bromoacetic acid-N-hydroxysuccinimide ester as described in U.S. Pat. No. 5,237,016. U.S. Pat. No. 5,237,016 also describes methods for preparing thiol-derivatized nucleotides, which can then be reacted with thiol groups on the selected growth factor. Briefly, thiol-derivatized nucleotides are prepared using a 5′-phosphorylated nucleotide in two steps: (1) reaction of the phosphate group with imidazole in the presence of a diimide and displacement of the imidazole leaving group with cystamine in one reaction step; and (2) reduction of the disulfide bond of the cystamine linker with dithiothreitol (see, also, Chu et al. (1988) Nucl.

Acids Res. 16:3671-3691, which describes a similar procedure). The 5′-phosphorylated starting oligonucleotides can be prepared by methods known to those of skill in the art (see, e.g., Maniatis et al. (1982) Molecular Cloning: A Laboratory Manual, Cold Spring Harbor Laboratory, New York, p. 122).

The antisense oligomer or nucleic acid, such as a methylphosphonate oligonucleotide (MP-oligomer), may be derivatized by reaction with SPDP or SMPB. The resulting MP-oligomer may be purified by HPLC and then coupled to the chemokine receptor targeting agent. The MP-oligomer (about 0.1 μM) is dissolved in about 40-50 μl of 1:1 acetonitrile/water to which phosphate buffer (pH 7.5, final concentration 0.1 M) and a 1 mg MP-oligomer in about 1 mL phosphate buffered saline is added. The reaction is allowed to proceed for about 5-10 hours at room temperature and is then quenched with about 15 μL 0.1 iodoacetamide. The conjugates can be purified on heparin sepharose Hi Trap columns (1 mL, Pharmacia) and eluted with a linear or step gradient. The conjugate should elute in 0.6 M NaCl.

b. Linkers

The linker, L, attaches the antibody to a targeted agent through covalent bond(s). The linker is a bifunctional or multifunctional moiety which can be used to link one or more targeted agent(s) to the anti-EGFR antibody to form an antibody-drug conjugate (ADC). ADCs can be readily prepared using a linker having reactive functionality for binding to the targeted agent and to the anti-EGFR antibody. A cysteine thiol group, or an amine group, e.g., N-terminus or lysine side chain, of the anti-EGFR antibody can form a bond with a functional group of a linker reagent, targeted agent or targeted agent-linker reagent.

Linkers are preferably stable in the extracellular environment so that the antibody-drug conjugate (ADC) is stable and remains intact, i.e., the antibody remains linked to the targeted agent, before transport or delivery into the target cell. Thus, the linkers are stable outside the target cell and may be cleaved or enable dissociation of the antibody and targeted agent at some efficacious rate once inside the cell. Contemplated linkers will (i) not interfere with the specific binding properties of the antibody; (ii) permit intracellular delivery of the conjugate or targeted agent; (iii) remain stable and intact, i.e., not cleaved, until the conjugate has been delivered or transported to its targeted site; and (iv) not interfere with the cytotoxic, cell-killing effect or a cytostatic effect of the targeted agent. Stability of the ADC may be measured by standard analytical techniques such as mass spectrometry and/or HPLC.

Linkers have two reactive functional groups to permit covalent attachment to both the antibody and the targeted agent, and thus exhibit bivalency in a reactive sense. Such chemical cross-linking reagents, which are useful for attaching two or more functional or biologically active moieties, such as peptides, nucleic acids, drugs, toxins, antibodies, haptens, and reporter groups, are known, and methods have been described for their use in generating conjugates (Hermanson, G. T. (1996) Bioconjugate Techniques; Academic Press: New York, p 234-242).

In some examples, a linker has a reactive functional group which has a nucleophilic group that is reactive to an electrophilic group present on an antibody. Useful electrophilic groups on an antibody include, but are not limited to, aldehyde and ketone carbonyl groups. The heteroatom of a nucleophilic group of a linker can react with an electrophilic group on an antibody and form a covalent bond to an antibody unit. Useful nucleophilic groups on a linker include, but are not limited to, hydrazide, oxime, amino, hydrazine, thiosemicarbazone, hydrazine carboxylate, and arylhydrazide. The electrophilic group on an antibody provides a convenient site for attachment to a linker.

### i. Peptide Linkers

Linkers can be peptidic, comprising one or more amino acid units. Peptide linker reagents may be prepared by solid phase or liquid phase synthesis methods (E. Schroder and K. Lubke, The Peptides, volume 1, pp. 76-136 (1965) Academic Press) that are well-known in the field of peptide chemistry, including t-BOC chemistry (Geiser et al. “Automation of solid-phase peptide synthesis” in Macromolecular Sequencing and Synthesis, Alan R. Liss, Inc., 1988, pp. 199-218) and Fmoc/HBTU chemistry (Fields, G. and Noble, R. (1990) “Solid phase peptide synthesis utilizing 9-fluorenylmethoxycarbonyl amino acids”, Int. J. Peptide Protein Res. 35:161-214), on an automated synthesizer such as the Rainin Symphony Peptide Synthesizer (Protein Technologies, Inc.), or Model 433 (Applied Biosystems). Peptide-based linkers offer advantages over linkers that are hydrolytically or reductively labile, since proteolysis is enzymatic, and the enzymes can be selected for preferential expression within tumor cells. The cathepsin B-cleavable peptide linker, valine-citrulline (Val-Cit), and modifications thereof such as maleimidocaproyl-valine-citrulline (mc-vc), phenylalanine-lysine, Ala-Leu-Ala-Ala (SEQ ID NO: 351), other tri/tetrapeptides are exemplary peptide linkers that have been employed in ADCs (Dosio et al., (2010) Toxins 3:848-883; Doronina et al., (2006) Bioconjug Chem. 17:114-124; Doronina et al., (2003) Nat Biotechnol. 21:778-784; Sanderson et al., (2005) Clin Cancer Res 11:843-852; Ducry and Stump (2010) Bioconjug Chem. 21:5-13). Exemplary non-cleavable peptide linkers include N-methyl-valine-citrulline. Other peptide linkers are described in U.S. Publication No. 2011/0020343.

Preferred peptide linkers are those that can be incorporated in fusion proteins and expressed in a host cell, such as E. coli. Such linkers include: enzyme substrates, such as cathepsin B substrate, cathepsin D substrate, trypsin substrate, thrombin substrate, subtilisin substrate, Factor Xa substrate, and enterokinase substrate; linkers that increase solubility, flexibility, and/or intracellular cleavability include linkers, such as (glyₘser)ₙ and (serₘgly)ₙ, where m is 1 to 6, preferably 1 to 4, more preferably 2 to 4, and n is 1 to 6, preferably 1 to 4, more preferably 2 to 4 (see, e.g., International PCT application No. WO 96/06641, which provides exemplary linkers for use in conjugates). In some embodiments, several linkers may be included in order to take advantage of desired properties of each linker.

### ii. Chemical Linkers

ADCs also can be prepared using linkers that are non-cleavable moieties or chemical cross-linking reagents. Exemplary non-cleavable linkers include amide linkers and amide and ester linkages with succinate spacers (Dosio et al., (2010) Toxins 3:848-883). Exemplary chemical cross-linking linkers include, but are not limited to, SMCC (Succinimidyl-4-(N-maleimidomethyl)cyclohexane-1-carboxylate) and SIAB (Succinimidyl (4-iodoacetyl)aminobenzoate). SMCC is an amine-to-sulfhydryl crosslinker that contains NHS-ester and maleimide reactive groups at opposite ends of a medium-length cyclohexane-stabilized spacer arm. SIAB is a short, NHS-ester and iodoacetyl crosslinker for amine-to-sulfhydryl conjugation. Other exemplary cross-linking reagents include, but are not limited to, thioether linkers, chemically labile hydrazone linkers, 4-mercaptovaleric acid, BMPEO, BMPS, EMCS, GMBS, HBVS, LC-SMCC, MBS, MPBH, SBAP, SIA, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate), and bis-maleimide reagents, such as DTME, BMB, BMDB, BMH, BMOE, BM(PEO)₃, and BM(PEO)₄, which are commercially available (Pierce Biotechnology, Inc.). Bis-maleimide reagents allow the attachment of a free thiol group of a cysteine residue of an antibody to a thiol-containing targeted agent, or linker intermediate, in a sequential or concurrent fashion. Other thiol-reactive functional groups, besides maleimide, include iodoacetamide, bromoacetamide, vinyl pyridine, disulfide, pyridyl disulfide, isocyanate, and isothiocyanate. Other exemplary linkers and methods of use are described in U.S. Publication No. 2005/0276812 and in Ducry and Stump (2010) Bioconjug Chem. 21:5-13.

Linkers optionally can be substituted with groups which modulate solubility or reactivity. For example, a sulfonate substituent may increase water solubility of the reagent and facilitate the coupling reaction of the linker reagent with the antibody or the drug moiety, or facilitate the coupling reaction of the anti-EGFR Ab-L with the targeted agent, or targeted agent-L with the anti-EGFR Ab, depending on the synthetic route employed to prepare the ADC.

Other linker reagents can also be obtained via commercial sources, such as Molecular Biosciences Inc. (Boulder, Colo.), or synthesized in accordance with procedures described in Toki et al. (2002) J. Org. Chem. 67:1866-1872; U.S. Pat. No. 6,214,345; WO 02/088172; U.S. 2003130189; U.S. 2003096743; WO 03/026577; WO 03/043583; and WO 04/032828. For example, linker reagents such as DOTA-maleimide (4-maleimidobutyramidobenzyl-DOTA) can be prepared by the reaction of aminobenzyl-DOTA with 4-maleimidobutyric acid (Fluka) activated with isopropylchloroformate (Aldrich), following the procedure of Axworthy et al. (2000) Proc. Natl. Acad. Sci. USA 97(4):1802-1807). DOTA-maleimide reagents react with the free cysteine amino acids of the cysteine engineered antibodies and provide a metal complexing ligand on the antibody (Lewis et al. (1998) Bioconj. Chem. 9:72-86). Chelating linker labelling reagents such as DOTA-NHS (1,4,7,10-tetraazacyclododecane-1,4,7,10-tetraacetic acid mono (N-hydroxysuccinimide ester) are commercially available (Macrocyclics, Dallas, Tex.).

The Linker can be a dendritic type linker for covalent attachment of more than one drug moiety through a branching, multifunctional linker moiety to an antibody (Sun et al. (2002) Bioorganic & Medicinal Chemistry Letters 12:2213-2215; Sun et al. (2003) Bioorganic & Medicinal Chemistry 11:1761-1768; King et al. (2002) Tetrahedron Letters 43:1987-1990). Dendritic linkers can increase the molar ratio of targeted agent to antibody, i.e., loading, which can increase the potency of the ADC. Thus, where an antibody bears only one reactive cysteine thiol group, a multitude of drug moieties may be attached through a dendritic linker. Exemplary dendritic linker reagents are described in U.S. Patent Publication No. 2005/0276812.

c. Exemplary Conjugates

Provided herein are Y104E- and Y104D-anti-EGFR antibody conjugates containing any of the anti-EGFR antibodies provided herein linked directly or indirectly to a cytotoxic moiety that is an auristatin or maytansinoid. The antibody conjugates have the formula (Ab), (L)_{q}, and (targeted agent)ₘ, wherein antibody (Ab) is the variant Y104E- or Y104D-anti-EGFR antibody or antigen-binding fragment thereof that binds to EGFR; L is a linker for linking the Ab to the targeted agent; the targeted agent is an auristatin or maytansinoid, m is at least 1; q is 0 or more as long as the resulting conjugate binds to the EGFR. In some examples, m is 1 to 8 and q is 0 to 8. Typically, the orientation of components in the conjugate is Ab-[(L)_{q}-(targeted agent)ₘ], whereby the Ab is linked indirectly to the targeted agent, i.e. an auristatin or a maytansinoid, via a linker. Generally, m and q are each independently from 2 to 6. In particular examples, m and q are the same, such that the resulting conjugate has the formula Ab-[(L)-(targeted agent)ₚ], where p is from 2 to 6, such as generally at least or about 2, 3, 4, 5 or 6.

i. Anti-EGFR Antibody-Auristatin Conjugates

Provided herein are antibody conjugates Y104E- and Y104D-anti-EGFR antibody conjugates containing any of the anti-EGFR antibodies provided herein linked directly or indirectly to an auristatin cytotoxic moiety. In such examples, the antibody conjugate has the formula (Ab), (L)_{q}, and (auristatin)ₘ, wherein antibody (Ab) is the variant Y104E- or Y104D-anti-EGFR antibody or antigen-binding fragment thereof that binds to EGFR, L is a linker for linking the Ab to the auristatin, m is at least 1 (e.g. m is 1 to 8) and q is 0 or more (e.g. 0 to 8) as long as the resulting conjugate binds to the EGFR. Typically, the orientation of components in the conjugate is Ab-[(L)_{q}-(auristatin)ₘ], whereby the Ab is linked indirectly to the auristatin agent via a linker. In particular examples, m and q are the same, such that the resulting conjugate has the formula Ab-[(L)-(auristatin)]ₚ, where p is from 2 to 6, such as generally at least or about 2, 3, 4, 5 or 6.

In the examples, the antibody component can be any anti-EGFR antibody described herein, or antigen-binding fragment thereof. In one example, the antibody component can be a Y104E-variant antibody, such as any set forth in subsection C.1 and C.2 above. In particular, exemplary of a Y104E-variant anti-EGFR antibody in the auristatin-containing conjugates provided herein is an antibody containing the heavy chain set forth in SEQ ID NO: 72 and the light chain set forth in SEQ ID NO: 8, or an antigen-binding fragment thereof that contains the variable heavy chain corresponding to amino acids 1-119 of SEQ ID NO: 72 (i.e., set forth in SEQ ID NO: 74) and the variable light chain corresponding to amino acids 1-107 of SEQ ID NO: 8 (i.e. set forth in SEQ ID NO: 9), or antibodies that contain a heavy chain and/or light chain, or portion thereof, that exhibit at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity thereto and that contains the amino acid replacement Y104E in the variable heavy chain, or humanized forms thereof. For example, the Y104E-variant anti-EGFR antibody in the auristatin-containing conjugate contains variant Y104E antibody provided herein such as any set forth in Tables 7, 9 or Table 10, or an antigen-binding fragment thereof that contains the variable heavy chain corresponding to amino acids 1-119 of the respective heavy chain and the variable light chain corresponding to amino acids 1-107 of the respective light chain. As an example, the Y104E-variant anti-EGFR antibodies in the auristatin-containing conjugate contains a humanized Y104E antibody designated E-h containing the heavy chain set forth in SEQ ID NO: 59 and a light chain set forth in SEQ ID NO: 181, or an antigen-binding fragment thereof that contains the variable heavy chain corresponding to amino acids 1-119 of SEQ ID NO: 59 and a variable light chain corresponding to amino acids 1-107 of SEQ ID NO: 181. The antibody component can be a full-length antibody or an antigen-binding fragment, such as an Fab, Fab′, F(ab′)₂, single-chain Fv (scFv), Fv, dsFv, diabody, Fd and Fd′ fragment.

In another example, the antibody component can be a Y104D-variant antibody, such as any set forth in subsection C.3. In particular, exemplary of a Y104D-variant anti-EGFR antibody in the auristatin-containing conjugates provided herein is an antibody containing the heavy chain set forth in SEQ ID NO: 67 and the light chain set forth in SEQ ID NO: 8, or an antigen-binding fragment thereof that contains the variable heavy chain corresponding to amino acids 1-119 of SEQ ID NO: 67 and the variable light chain corresponding to amino acids 1-107 of SEQ ID NO: 8, or antibodies that contain a heavy chain and/or light chain, or portion thereof, that exhibit at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity thereto and that contains the amino acid replacement Y104D in the variable heavy chain, or humanized forms thereof. For example, exemplary of Y104D-variant anti-EGFR antibodies in the auristatin-containing conjugates provided herein are any set forth in Table 11, or an antigen-binding fragment thereof that contains the variable heavy chain corresponding to amino acids 1-119 of the respective heavy chain and the variable light chain corresponding to amino acids 1-107 of the respective light chain. As an example, the Y104D-variant anti-EGFR antibodies in the auristatin-containing conjugate contains a humanized Y104D antibody designated D-h containing the heavy chain set forth in SEQ ID NO: 57 and a light chain set forth in SEQ ID NO: 181, or an antigen-binding fragment thereof that contains the variable heavy chain corresponding to amino acids 1-119 of SEQ ID NO: 57 and a variable light chain corresponding to amino acids 1-107 of SEQ ID NO: 181. The antibody component can be a full-length antibody or an antigen-binding fragment, such as an Fab, Fab′, F(ab′)₂, single-chain Fv (scFv), Fv, dsFv, diabody, Fd and Fd′ fragment.

The auristatin in the conjugates can be any described known in the art, including any described in subsection C.4.a.ii. See also, published International PCT Application No. WO2012054748 and U.S. patent application No. US2011/0020343. In one example, the auristatin is MMAE that has the structure:

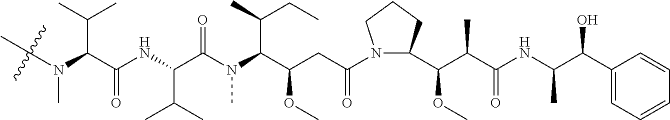

or a pharmaceutically acceptable salt form thereof. In another example, the auristatin is MMAF that has the structure:

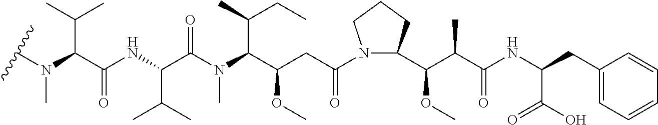 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)O)OC)C(C)C)C(C)C]

or a pharmaceutically acceptable salt form thereof.

In any of such examples, the linker can be any linker described above in subsection C.4.b. Typically, the linker is a linker that is capable of reacting with a sulfydryl group on the antibody. The linker can have the formula Aₐ-Y_{y}-Z_{z}-Xₓ or a pharmaceutically acceptable salt thereof, where A is a bridge unit capable of reacting with a sulfhydryl group of the antibody; a is 0 or 1; each of Y and Z is independently an amino acid unit; each of y and z is independently an integer ranging from 0 to 12; X is a Spacer unit; and X is 0, 1 or 2.

In such examples, the bridge unit is capable of linking the antibody to the amino acid Y or W, if present, to a Spacer Unit X, if present, or to the targeted agent. In such examples, a sulfhydryl group on the antibody is a functional group that can react with the bridge unit. In one example, the sulfhydryl group can be generated by reduction of the intramolecular disulfide bonds of the antibody. Disulfides can be reduced, for example, with dithiothreitol, mercaptoethanol, or tris(2-carboxyethyl)phosphine using standard methods. In another example, sulfhydryl group can be generated by reaction of an amino group of a lysine moiety of the antibody with 2-iminothiolane (Traut's reagent) or other sulfhydryl generating reagent. Reactive linkers that can form a bond with a sulfur atom of the antibody are known in the art (see e.g. published International PCT Application No. WO2012054748 and WO2005/007197).

In one example, the functional or reactive moiety of the bridge unit includes those that are broadly selective for thiol groups, such as iodoacetamide, maleimide, vinylsulfone, vinyl pyridines and acrylate and methacrylate esters. Such a thiol selective conjugating moieties can yield a single thioether conjugating bond with the antibody. For example, the bridge can contain a reactive group that is a maleimide. In particular examples, the bridge unit is maleimidocaproyl (MC) or maleimidopropanoyl (MP).b For example, the bridge contains a

In another example, the reactive moiety of the bridge unit is a bifunctional linker that maintains the interchain disulfide bonding of the antibody. For example, the bridge unit can contain a bifunctional pyrrole-2,5-dione- and pyrrolidine-2,5-dione-based linkers as described in published U.S. patent application No. US2013/0224228. In such an example, reaction of the bifunctional linker with the two cysteines gives a “stapled” dithiosuccinimide or dithiomaleimide antibody conjugate with one linker per disulfide connected through two thioether bonds. In another example, a bifunctional linker can be a bis-thiol alkylating reagent as described in International PCT Application No. WO2005/007197. The bis-thiol alkylating reagent can undergo bis-alkylation to link to both cysteine thiols derived from the reduced disulfide. Once formed, the reagent can undergo interactive Michael and retro-Michael reactions to allow the product to be formed in which two free thiols can re-anneal across a 3-carbon bridge. In one example, the bis-thiol alkylating reagent has the structure:

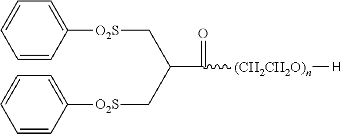

where, (CH₂CH₂O)ₙ is a hydrophilic PEG spacer. For example, the bis-thiol alkylating reagent can be 4-(3-tosyl-2-(tosylmethyl)propanoyl)benzamide-PEG having the structure:

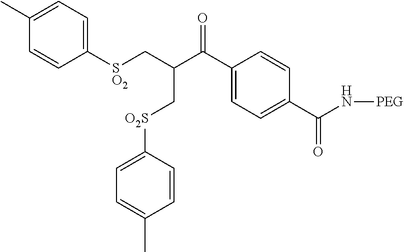

In any of the examples of an auristatin-based conjugate, the amino acid Y and Z, if each independently present, can be a natural or non-natural amino acid. For example, Y and Z, if each independently present, can be an amino acid that is alanine, valine, leucine, isoleucine, methionine, phenylalanine, tryptophan or proline. The Y-Z can represent an amino acid unit that is a dipeptide, tripeptide, tetrapeptide, pentapeptide or higher unit peptide. The amino acid unit can be enzymatically cleaved by one or more enzymes, including a cancer or tumor-associated protease, to liberate the targeted agent. For example, the Y can be an amino acid that is alanine, valine, leucine, isoleucine, methionine, phenylalanine, tryptophan or proline and the Z can be lysine, lysine protected with acetyl or formyl, arginine, arginine protected with tosyl or nitro groups, histidine, ornithine, ornithine protected with acetyl or formyl or citrulline. In particular examples, Y-Z is phenylalanine-lysine, valine-citrulline or valine-lysine.

In the linker, X is a spacer unit. The spacer unit can be non-self-immolative or self-immolative. A non-self-immolative Spacer unit is one in which part or all of the Spacer unit remains bound to the targeted agent after cleavage, for example enzymatic cleavage of an amino acid unit Y-Z, from the antibody drug conjugate. Examples of a non-self-immolative Spacer unit include, but are not limited to a (glycine-glycine) Spacer unit and a glycine Spacer unit. In some examples, Spacer unit (—X—) is -Gly-. In other examples, Spacer unit (—X—) is -Gly-Gly-. In other cases, the Spacer unit is a bifunctional chemical moiety that is capable of covalently linking together two spaced chemical moieties into a stable tripartite molecule. It will spontaneously separate from the second chemical moiety if its bond to the first moiety is cleaved. For example, the Spacer Unit (—X—)can be a p-aminobenzyl alcohol (PAB). The Spacer Unit (—X—) also can be an aromatic compound that is electronically similar to PAB, such as 2-aminoimidazol-5-methanol derivatives (Hay et al. (1999) Bioorg. Med. Chem. Lett. 9:2237) and ortho or para-aminobenzylacetals. Spacers can be used that undergo cyclization upon amide bond hydrolysis, such as substituted and unsubstituted 4-aminobutyric acid amides (Rodrigues et ah (1995) Chemistry Biology 2:223), appropriately substituted bicyclo[2.2.1] and bicyclo[2.2.2] ring systems (Storm et. al (1972). Amer. Chem. Soc. 94:5815) and 2-aminophenylpropionic acid amides (Amsberry et. al (1990) Org. Chem. 55:5867). In particular, the spacer contains a p-amino benzyl having the following structure:

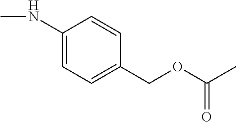

In particular examples of the conjugates provided herein, the linker L is 6-maleimidocaproyl-valine-citrulline-p-aminobenzyloxycarbonyl (MC-vc-PAB) or 4-(3-tosyl-2-(tosylmethyl)propanoyl)benzamide-valine-citruline-p-aminobenzyloxycarbonyl.

In one example, among the Y104D- or Y104E-variant anti-EGFR antibody conjugates provided herein is a conjugate in which the targeted agent is MMAF and L-(targeted agent) has the structure:

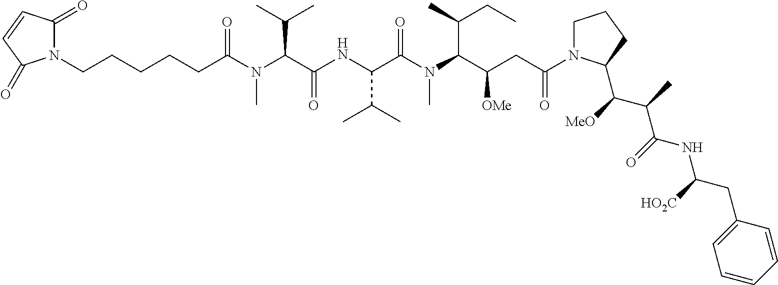 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)C=CC1=O)C(C)C]

In another example, among the Y104D- or Y104E-variant anti-EGFR antibody conjugates provided herein is a conjugate in which the targeted agent is MMAE and L-(targeted agent) has the structure:

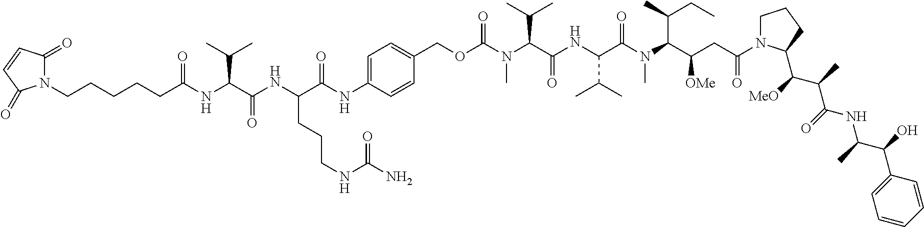 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)C(CCCNC(N)=O)NC(=O)[C@@H](NC(=O)CCCCCN2C(=O)C=CC2=O)C(C)C)cc1)C(C)C]

In a further example, among the Y104D- or Y104E-variant anti-EGFR antibody conjugates provided herein is a conjugate in which the targeted agent is MMAE and L-(targeted agent) has the structure:

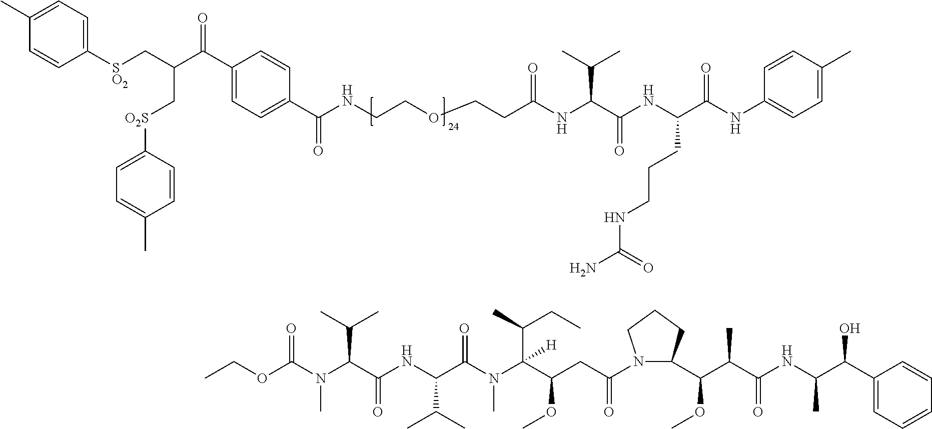

In any of such examples, m and q are the same and the conjugate has the formula: Ab-[(L)-(targeted agent)]ₚ. For example, p can be 2 to 6, such as generally about or at least 2, 3, 4, 5 or 6. The final conjugate contains 2 to 6 auristatin (e.g. MMAE or MMAF) molecules per antibody, such as generally about or at least 2, 3, 4, 5 or 6.

ii. Anti-EGFR Antibody-Maytansinoid Conjugates

Provided herein are antibody conjugates Y104E- and Y104D-anti-EGFR antibody conjugates containing any of the anti-EGFR antibodies provided herein linked directly or indirectly to a maytansinoid cytotoxic moiety. In such examples, the antibody conjugate has the formula (Ab), (L)_{q}, and (maytansinoid)ₘ, wherein antibody (Ab) is the variant Y104E- or Y104D-anti-EGFR antibody or antigen-binding fragment thereof that binds to EGFR, L is a linker for linking the Ab to the maytansinoid, m is at least 1 (e.g. m is 1 to 8) and q is 0 or more (e.g. 0 to 8) as long as the resulting conjugate binds to the EGFR. Typically, the orientation of components in the conjugate is Ab-[(L)_{q}-(maytansinoid)ₘ], whereby the Ab is linked indirectly to the maytansinoid agent via a linker. In particular examples, m and q are the same, such that the resulting conjugate has the formula Ab-[(L)-(maytansinoid)]ₚ, where p is from 2 to 6, such as generally at least or about 2, 3, 4, 5 or 6.

In the examples, the antibody component can be any anti-EGFR antibody described herein, or antigen-binding fragment thereof. In one example, the antibody component can be a Y104E-variant antibody, such as any set forth in subsection C.1 and C.2 above. In particular, exemplary of a Y104E-variant anti-EGFR antibody in the maytansinoid-containing conjugates provided herein is an antibody containing the heavy chain set forth in SEQ ID NO: 72 and the light chain set forth in SEQ ID NO: 8, or an antigen-binding fragment thereof that contains the variable heavy chain corresponding to amino acids 1-119 of SEQ ID NO: 72 (i.e., set forth in SEQ ID NO: 74) and the variable light chain corresponding to amino acids 1-107 of SEQ ID NO: 8 (i.e., set forth in SEQ ID NO: 9), or antibodies that contain a heavy chain and/or light chain, or portion thereof, that exhibit at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity thereto and that contains the amino acid replacement Y104E in the variable heavy chain, or humanized forms thereof. For example, the Y104E-variant anti-EGFR antibody in the maytansinoid-containing conjugate contains variant Y104E antibody provided herein such as any set forth in Tables 7, 9 or Table 10, or an antigen-binding fragment thereof that contains the variable heavy chain corresponding to amino acids 1-119 of the respective heavy chain and the variable light chain corresponding to amino acids 1-107 of the respective light chain. As an example, the Y104E-variant anti-EGFR antibodies in the maytansinoid-containing conjugate contains a humanized Y104E antibody designated E-h containing the heavy chain set forth in SEQ ID NO: 59 and a light chain set forth in SEQ ID NO: 181, or an antigen-binding fragment thereof that contains the variable heavy chain corresponding to amino acids 1-119 of SEQ ID NO: 59 and a variable light chain corresponding to amino acids 1-107 of SEQ ID NO: 181. The antibody component can be a full-length antibody or an antigen-binding fragment, such as an Fab, Fab′, F(ab′)₂, single-chain Fv (scFv), Fv, dsFv, diabody, Fd and Fd′ fragment.

In another example, the antibody component can be a Y104D-variant antibody, such as any set forth in subsection C.3. In particular, exemplary of a Y104D-variant anti-EGFR antibody in the maytansinoid-containing conjugates provided herein is an antibody containing the heavy chain set forth in SEQ ID NO: 67 and the light chain set forth in SEQ ID NO: 8, or an antigen-binding fragment thereof that contains the variable heavy chain corresponding to amino acids 1-119 of SEQ ID NO: 67 and the variable light chain corresponding to amino acids 1-107 of SEQ ID NO: 8, or antibodies that contain a heavy chain and/or light chain, or portion thereof, that exhibit at least 75%, 80%, 81%, 82%, 83%, 84%, 85%, 86%, 86%, 88%, 89%, 90%, 91%, 92%, 93%, 94%, 95%, 96%, 97%, 98%, 99% or more sequence identity thereto and that contains the amino acid replacement Y104D in the variable heavy chain, or humanized forms thereof. For example, exemplary of Y104D-variant anti-EGFR antibodies in the maytansinoid-containing conjugates provided herein are any set forth in Table 11, or an antigen-binding fragment thereof that contains the variable heavy chain corresponding to amino acids 1-119 of the respective heavy chain and the variable light chain corresponding to amino acids 1-107 of the respective light chain. As an example, the Y104D-variant anti-EGFR antibodies in the maytansinoid-containing conjugate contains a humanized Y104D antibody designated D-h containing the heavy chain set forth in SEQ ID NO: 57 and a light chain set forth in SEQ ID NO: 181, or an antigen-binding fragment thereof that contains the variable heavy chain corresponding to amino acids 1-119 of SEQ ID NO: 57 and a variable light chain corresponding to amino acids 1-107 of SEQ ID NO: 181. The antibody component can be a full-length antibody or an antigen-binding fragment, such as an Fab, Fab′, F(ab′)₂, single-chain Fv (scFv), Fv, dsFv, diabody, Fd and Fd′ fragment.

The maytansinoid in the conjugates can be any known in the art, including any described in subsection C.4.a.i See also, U.S. Pat. No. 7,097,840; EP1928503. Maytansinoids are well known in the art and can be synthesized by known techniques or isolated from natural sources. Suitable maytansinoids are disclosed, for example, in U.S. Pat. No. 5,208,020 and in the other patents and nonpatent publications referred to hereinabove. In particular, maytansinoids are maytansinol and maytansinol analogs modified in the aromatic ring or at other positions of the maytansinol molecule, such as various maytansinol esters.

Exemplary of a maytansinoid is DM1 having the following structure:

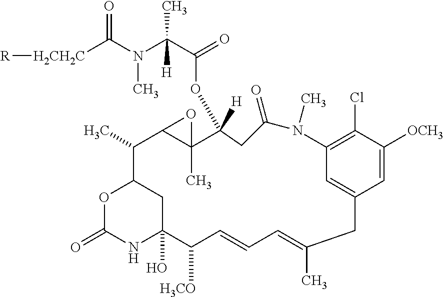

In the structure, “R” can be occupied by a variant of groups capable of forming a chemical bond with a selected linker. For example, “R” can be SH or can be SSR₁, where R₁ represents methyl, linear alkyl, branched alkyl, cyclic alkyl, simple or substituted aryl or heterocyclic. Typically, “R” is an SH group or a protected derivative thereof, which forms an S—S bond with a linker. For example,
to form the maytansinoid DM1, the side chain at the C-3 hydroxyl group of maytansine is modified to have a free sulfhydryl group (SH). This thiolated form of maytansine can react with a modified antibody to form a conjugate.

In any of such examples, the linker can be any linker described above in subsection C.4.b. The linker typically is a bifunctional crosslinking agent, and the antibody is modified by reacting the bifunctional crosslinking reagent with the antibody, thereby resulting in the covalent attachment of a linker molecule to the antibody. Exemplary linkers include, but are not limited to, N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, linkers can include N-succinimidyl-3-(2-pyridyldithio) propionate (SPDP) (Carlsson et al., Biochem. J. 173:723-737 [1978]) and N-succinimidyl-4-(2-pyridylthio)pentanoate (SPP) to provide for a disulfide linkage. The linker can be a cleavable or non-cleavable linker. Exemplary of a non-cleavable linker succinimidyl-4-(N-maleimidomethyl)cyclohexane-1-carboxylate (SMCC).

For example, among the Y104D- or Y104E-variant anti-EGFR antibody conjugates provided herein is a conjugate in which the targeted agent is DM1 and L-(targeted agent) has the structure:

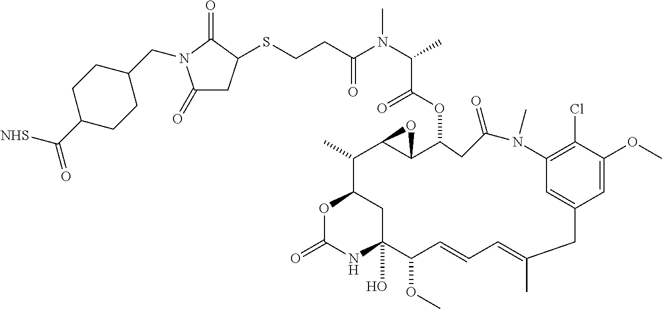

In such examples, m and q are the same and the conjugate has the formula: Ab-[(L)-(targeted agent)]ₚ. For example, p can be 2 to 6, such as generally about or at least 2, 3, 4, 5 or 6. The final conjugate contains 2 to 6 DM1 molecules per antibody, such as generally about or at least 2, 3, 4, 5 or 6.

### D. METHODS OF PRODUCING ANTI-EGFR ANTIBODIES

1. Generating and Producing Anti-EGFR Antibodies

Anti-EGFR antibodies, such as the modified anti-EGFR antibodies provided herein, can be expressed using standard cell culture and other expression systems known in the art. Prior to use in the methods provided herein, the proteins can be purified. Alternatively, whole supernatant or diluted supernatant can be used in the methods provided herein. The modified anti-EGFR antibodies provided herein can be produced by recombinant DNA methods that are within the purview of those skilled in the art. DNA encoding a modified anti-EGFR antibody can be synthetically produced or can be readily isolated and sequenced using conventional procedures (e.g., by using oligonucleotide probes that are capable of binding specifically to genes encoding the heavy and light chains of the antibody). For example, any cell source known to produce or express a modified anti-EGFR antibody can serve as a preferred source of such DNA. In another example, once the sequence of the DNA encoding the modified anti-EGFR is determined, nucleic acid sequences can be constructed using gene synthesis techniques.

Further, mutagenesis techniques also can be employed to generate further modified forms of an anti-EGFR antibody. The DNA also can be modified. For example, gene synthesis or routine molecular biology techniques can be used to effect insertion, deletion, addition or replacement of nucleotides. For example, additional nucleotide sequences can be joined to a nucleic acid sequence. In one example linker sequences can be added, such as sequences containing restriction endonuclease sites for the purpose of cloning the antibody gene into a vector, for example, a protein expression vector. Furthermore, additional nucleotide sequences specifying functional DNA elements can be operatively linked to a nucleic acid molecule. Examples of such sequences include, but are not limited to, promoter sequences designed to facilitate intracellular protein expression, and leader peptide sequences designed to facilitate protein secretion.

It is understood that any of the amino acid sequences provided herein can be reverse-translated, using standard methods commonly used by those skilled in the art, to generate corresponding encoding nucleic acid sequences, which can be cloned into vectors and expressed to generate the antibodies and fragments provided herein. Anti-EGFR antibodies, such as the modified anti-EGFR antibodies provided herein, can be expressed as full-length proteins or less than full length proteins. For example, antibody fragments can be expressed. Nucleic acid molecules and proteins provided herein can be made by any method known to one of skill in the art. Such procedures are routine and are well-known to the skill artisan. They include routine molecular biology techniques including gene synthesis, PCR, ligation, cloning, transfection and purification techniques. A description of such procedures is provided below.

Once isolated, the DNA can be placed into expression vectors, which are then transfected into host cells. The choice of vector can depend on the desired application. For example, after insertion of the nucleic acid, the vectors typically are used to transform host cells, for example, to amplify the protein genes for replication and/or expression thereof. In such examples, a vector suitable for high level expression is used.

For expression of antibodies, generally, nucleic acid encoding the heavy chain of an antibody is cloned into a vector and the nucleic acid encoding the light chain of an antibody is cloned into a vector. The genes can be cloned into a single vector for dual expression thereof, or into separate vectors. If desired, the vectors also can contain further sequences encoding additional constant region(s) or hinge regions to generate other antibody forms. The vectors can be transfected and expressed in host cells. Expression can be in any cell expression system known to one of skill in the art. For example, host cells include cells that do not otherwise produce immunoglobulin protein, to obtain the synthesis of antibodies in the recombinant host cells. For example, host cells include, but are not limited, to simian COS cells, Chinese hamster ovary (CHO) cells, 293FS cells, HEK293-6E cells, NSO cells or other myeloma cells. Other expression vectors and host cells are described herein.

The modified anti-EGFR antibodies provided herein can be generated or expressed as full-length antibodies or as antibodies that are less than full length, including, but not limited to, antigen-binding fragments, such as, for example, Fab, Fab′, Fab hinge, F(ab′)₂, single-chain Fv (scFv), scFv tandem, Fv, dsFv, scFv hinge, scFv hinge(ΔE) diabody, Fd and Fd′ fragments. Various techniques have been developed for the production of antibody fragments. For example, fragments can be derived via proteolytic digestion of intact antibodies (see, e.g., Morimoto et al. (1992) Journal of Biochemical and Biophysical Methods, 24:107-117; Brennan et al. (1985) Science, 229:81). Alternatively, fragments can be produced directly by recombinant host cells. For example, Fab, Fv and scFv antibody fragments can all be expressed in and secreted from host cells, such as E. coli, thereby facilitating production of large amounts of these fragments. F(ab′)₂ fragments can be produced by chemically coupling Fab′-SH fragments (Carter et al. (1992) Bio/Technology, 10:163-167), or they can be isolated directly from recombinant host cell culture. In some examples, the modified anti-EGFR antibody is a single chain Fv fragment (scFv) (e.g., WO 93/16185; U.S. Pat. Nos. 5,571,894 and 5,587,458). Fv and scFv fragments have intact combining sites but are devoid of constant regions; thus, they are suitable for reduced nonspecific binding during in vivo use. scFv fusion proteins can be constructed to attach an effector protein at either the amino- or the carboxy-terminus of an scFv. The antibody fragment can also be a linear antibody (see, e.g., U.S. Pat. No. 5,641,870). Such linear antibody fragments can be monospecific or bispecific. Other techniques for the production of antibody fragments are known to one of skill in the art.

Upon expression, antibody heavy and light chains, or fragment(s) thereof, pair by interchain disulfide bonds to form a full-length antibody or fragment thereof. For example, for expression of a full-length Ig, sequences encoding the V_{H}-C_{H}1-hinge-C_{H}2-C_{H}3 can be cloned into a first expression vector and sequences encoding the V_{L}-C_{L} domains can be cloned into a second expression vector. Upon co-expression, the full-length heavy and light chains are interlinked by disulfide bonds to generate a full-length antibody. In another example, to generate a Fab, sequences encoding a fragment containing the V_{H} and C_{H}1 regions can be cloned into a first expression vector and sequences encoding the V_{L}-C_{L} domains can be cloned into a second expression vector. Upon co-expression, the heavy chain pairs with a light chain to generate a Fab monomer.

Exemplary sequences that can be inserted into vectors for expression of whole antibodies and antibody fragments include nucleotide sequences which encode the corresponding heavy chain or light chain or fragments of any of the modified anti-EGFR antibodies provided herein. For example, the nucleotide sequences encoding any of the variable heavy chain and variable light chain sequences of any antibody or fragment described herein can be inserted into a suitable expression vector described herein or known to one of skill in the art. Any of the amino acid sequences of the modified anti-EGFR antibodies and EGFR-binding fragments provided herein can be reverse translated (also called back translated) to generate nucleic acid sequences, such as DNA sequences that encode the protein, using standard procedures. For example, there are several on-line tools are available to convert protein sequences to encoding DNA sequences, such as bioinformatics.org/sms2/rev_trans.html; biophp.org/minitools/protein_to_dna/demo.php; vivo.colostate.edu/molkit/rtranslate/; ebi.ac.uk/Tools/st/emboss_backtranseq/; molbiol.ru/eng/scripts/01_{—}19.html; and geneinfinity.org/sms/smsbacktranslation.html. Such reverse translated sequences can be inserted into any of the expression vectors provided herein for the expression and production of the provided antibodies or fragments.

In particular, a sequence of nucleotides encoding a modified anti-EGFR antibody with a 104E amino acid replacement has a sequence of nucleotides encoding a variable heavy chain set forth in SEQ ID NO: 73 and a sequence of nucleotides encoding the variable light chain set forth in SEQ ID NO: 51. Other non-limiting examples of sequences of nucleic acids encoding the variable heavy and light chains, which can be inserted into a suitable expression vector, are set forth in Table 10 and include those encoding a variable heavy chain having a sequence of nucleotides set forth in SEQ ID NOS: 60, 62, 130, 132, 136, 138, 142, 144, 148, 150, 210, 212, 216, 218, 222, 224, 228, 230, 234, 236, 240, 242, 246, 248, and any degenerate sequence thereof and those encoding a variable light chain having a sequence of nucleotides set forth in SEQ ID NOS: 154, 157, 161, 164, 168, 171, 175, 178, 182, 185, 189, 192, 196, 199, 203, 206, 252, 255, 259, 262, 266, 269, 273, 276, 280, 283, 287, 290, 294, 297, 301, 304 and any degenerate sequence thereof

For purposes herein with respect to expression of anti-EGFR antibodies, such as modified anti-EGFR antibodies, vectors can contain a sequence of nucleotides that encodes a constant region of an antibody operably linked to the nucleic acid sequence encoding the variable region of the antibody. The vector can include the sequence for one or all of a C_{H}1, C_{H}2, hinge, C_{H}3 or C_{H}4 and/or C_{L}. Generally, such as for expression of Fabs, the vector contains the sequence for a C_{H}1 or C_{L} (kappa or lambda light chains). The sequences of constant regions or hinge regions are known to one of skill in the art (see, e.g., U.S. Published Application No. 20080248028). Examples of such sequences are provided herein.

All or a portion of the constant region of the heavy chain or light chain also can be inserted or contained in the vector for expression of IgG antibodies or fragments thereof. For example, non-limiting examples include those encoding a full-length heavy chain having a sequence of nucleotides set forth in SEQ ID NOS: 58, 71, 128, 134, 140, 146, 208, 214, 220, 226, 232, 238, 244, and any degenerate sequence thereof and those encoding a variable light having a sequence of nucleotides set forth in SEQ ID NOS: 50, 152, 159, 166, 173, 180, 187, 194, 201, 250, 257, 264, 271, 278, 285, 292, 299 or degenerates thereof.

In addition, V_{H}—C_{H}1 and V_{L}—C_{L} sequences can be inserted into a suitable expression vector for expression of Fab molecules. Nucleic acids encoding variable heavy chain and variable light chain domains of an antibody can be expressed in a suitable expression vector, such as a vector encoding for a linker between the variable heavy chain and variable light chain to produce single chain antibodies. Exemplary linkers include the glycine rich flexible linkers (-G₄S—)ₙ, where n is a positive integer, such as 1 (SEQ ID NO: 346), 2 (SEQ ID NO: 347), 3 (SEQ ID NO: 46), 4 (SEQ ID NO: 348), 5 (SEQ ID NO: 349), or more.

a. Vectors

Choice of vector can depend on the desired application. Many expression vectors are available and known to those of skill in the art for the expression of anti-EGFR antibodies or portions thereof, such as antigen binding fragments. The choice of an expression vector is influenced by the choice of host expression system. Such selection is well within the level of skill of the skilled artisan. In general, expression vectors can include transcriptional promoters and optionally enhancers, translational signals, and transcriptional and translational termination signals. Expression vectors that are used for stable transformation typically have a selectable marker which allows for selection and maintenance of the transformed cells. In some cases, an origin of replication can be used to amplify the copy number of the vectors in the cells. Vectors also generally can contain additional nucleotide sequences operably linked to the ligated nucleic acid molecule (e.g., His tag, Flag tag). For applications with antibodies, vectors generally include sequences encoding the constant region. Thus, antibodies or portions thereof also can be expressed as protein fusions. For example, a fusion protein can be generated to add additional functionality to a polypeptide. Examples of fusion proteins include, but are not limited to, fusions of a signal sequence, an epitope tag such as for localization, e.g., a His₆ tag or a myc tag, or a tag for purification, such as a GST tag, and/or a sequence for directing protein secretion and/or membrane association.

For example, expression of the anti-EGFR antibodies, such as modified anti-EGFR antibodies, can be controlled by any promoter/enhancer known in the art. Suitable bacterial promoters are well-known in the art and described herein below. Other suitable promoters for mammalian cells, yeast cells and insect cells are well-known in the art and some are exemplified below. Selection of the promoter used to direct expression of a heterologous nucleic acid depends on the particular application. Promoters which can be used include but are not limited to eukaryotic expression vectors containing the SV40 early promoter (Bernoist and Chambon, Nature 290:304-310 (1981)), the promoter contained in the 3′ long terminal repeat of Rous sarcoma virus (Yamamoto et al. Cell 22:787-797 (1980)), the herpes thymidine kinase promoter (Wagner et al., Proc. Natl. Acad. Sci. USA 78:1441-1445 (1981)), the regulatory sequences of the metallothionein gene (Brinster et al., Nature 296:39-42 (1982)); prokaryotic expression vectors such as the β-lactamase promoter (Jay et al., (1981) Proc. Natl. Acad. Sci. USA 78:5543) or the tac promoter (DeBoer et al., Proc. Natl. Acad. Sci. USA 80:21-25 (1983)); see also “Useful Proteins from Recombinant Bacteria”: in Scientific American 242:74-94 (1980)); plant expression vectors containing the nopaline synthetase promoter (Herrera-Estrella et al., Nature 303:209-213 (1983)) or the cauliflower mosaic virus 35S RNA promoter (Gardner et al., Nucleic Acids Res. 9:2871 (1981)), and the promoter of the photosynthetic enzyme ribulose bisphosphate carboxylase (Herrera-Estrella et al., Nature 310:115-120 (1984)); promoter elements from yeast and other fungi such as the Ga14 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 38:639-646 (1984); Ornitz et al., Cold Spring Harbor Symp. Quant. Biol. 50:399-409 (1986); MacDonald, Hepatology 7:425-515 (1987)); insulin gene control region which is active in pancreatic beta cells (Hanahan et al., Nature 315:115-122 (1985)), immunoglobulin gene control region which is active in lymphoid cells (Grosschedl et al., Cell 38:647-658 (1984); Adams et al., Nature 318:533-538 (1985); Alexander et al., Mol. Cell Biol. 7:1436-1444 (1987)), mouse mammary tumor virus control region which is active in testicular, breast, lymphoid and mast cells (Leder et al., Cell 45:485-495 (1986)), albumin gene control region which is active in liver (Pinkert et al., Genes and Devel. 1:268-276 (1987)), alpha-fetoprotein gene control region which is active in liver (Krumlauf et al., Mol. Cell. Biol. 5:1639-1648 (1985); Hammer et al., Science 235:53-58 1987)), alpha-1 antitrypsin gene control region which is active in liver (Kelsey et al., Genes and Devel. 1:161-171 (1987)), beta globin gene control region which is active in myeloid cells (Magram et al., Nature 315:338-340 (1985); Kollias et al., Cell 46:89-94 (1986)), myelin basic protein gene control region which is active in oligodendrocyte cells of the brain (Readhead et al., Cell 48:703-712 (1987)), myosin light chain-2 gene control region which is active in skeletal muscle (Shani, Nature 314:283-286 (1985)), and gonadotrophic releasing hormone gene control region which is active in gonadotrophs of the hypothalamus (Mason et al., Science 234:1372-1378 (1986)).

In addition to the promoter, the expression vector typically contains a transcription unit or expression cassette that contains all the additional elements required for the expression of the antibody, or portion thereof, in host cells. A typical expression cassette contains a promoter operably linked to the nucleic acid sequence encoding the protein and signals required for efficient polyadenylation of the transcript, ribosome binding sites and translation termination. Additional elements of the cassette can include enhancers. In addition, the cassette typically contains a transcription termination region downstream of the structural gene to provide for efficient termination. The termination region can be obtained from the same gene as the promoter sequence or can be obtained from different genes.

Some expression systems have markers that provide gene amplification such as thymidine kinase and dihydrofolate reductase. Alternatively, high yield expression systems not involving gene amplification are also suitable, such as using a baculovirus vector in insect cells, with a nucleic acid sequence encoding a protein under the direction of the polyhedron promoter or other strong baculovirus promoter.

Exemplary expression vectors include any mammalian expression vector such as, for example, pCMV and pCDNA3.1. Other eukaryotic vectors, for example any containing regulatory elements from eukaryotic viruses can be used as eukaryotic expression vectors. These include, for example, SV40 vectors, papilloma virus vectors, and vectors derived from Epstein-Bar virus. Exemplary eukaryotic vectors include pMSG, pAV009/A+, pMT010/A+, pMAMneo-5, baculovirus pDSCE, and any other vector allowing expression of proteins under the direction of the CMV promoter, SV40 early promoter, SV40 late promoter, metallothionein promoter, murine mammary tumor virus promoter, Rous sarcoma virus promoter, polyhedron promoter, or other promoters shown effective for expression in eukaryotes. For bacterial expression, such vectors include pBR322, pUC, pSKF, pET23D, and fusion vectors such as MBP, GST and LacZ.

Any methods known to those of skill in the art for the insertion of DNA fragments into a vector can be used to construct expression vectors containing a nucleic acid encoding a protein or an antibody chain. These methods can include in vitro recombinant DNA and synthetic techniques and in vivo recombinants (genetic recombination). The insertion into a cloning vector can, for example, be accomplished by ligating the DNA fragment into a cloning vector which has complementary cohesive termini. If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized nucleic acids encoding restriction endonuclease recognition sequences.

In one example, nucleic acid encoding the heavy chain of an antibody, is ligated into a first expression vector and nucleic acid encoding the light chain of an antibody is ligated into a second expression vector. The expression vectors can be the same or different, although generally they are sufficiently compatible to allow comparable protein expression of the heavy and light chains. The first and second expression vectors are generally co-transfected into host cells, typically at a 1:1 ratio. Exemplary vectors include, but are not limited to, pγ1HC and pκLC (Tiller et al. (2008) J Immunol. Methods, 329:112-24). Other expression vectors include the light chain expression vector pAG4622 and the heavy chain expression vector pAH4604 (Coloma et al. (1992) J Immunol. Methods, 152:89-104). The pAG4622 vector contains the genomic sequence encoding the C-region domain of the human κ L chain and the gpt selectable marker. The pAH4604 vector contains the hisD selectable marker and sequences encoding the human H chain γ1 C-region domain. In another example, the heavy and light chain can be cloned into a single vector that has expression cassettes for both the heavy and light chain.

In some examples, the vector is a bicistronic vector that contains an internal ribosomal entry site (IRES) between the open reading frames encoding the heavy and light chains. For example, an exemplary vector includes the vector designated pcDNA3-Erbitux-LC-IRES-HC-WT (e.g., SEQ ID NO: 306), where nucleic acid encoding the heavy chain (HC) or light chain (LC) of any of the modified anti-EGFR antibodies provided herein can be substituted in place of the sequences therein. Examples of such vectors are set forth in any of SEQ ID NOS: 307-314.

b. Cells and Expression Systems

Generally, any cell type that can be engineered to express heterologous DNA and has a secretory pathway is suitable for expression of the modified anti-EGFR antibodies provided herein. Expression hosts include prokaryotic and eukaryotic organisms such as bacterial cells (e.g., E. coli), yeast cells, fungal cells, Archaea, plant cells, insect cells and animal cells including human cells. Expression hosts can differ in their protein production levels as well as the types of post-translational modifications that are present on the expressed proteins. Further, the choice of expression host is often related to the choice of vector and transcription and translation elements used. For example, the choice of expression host is often, but not always, dependent on the choice of precursor sequence utilized. For example, many heterologous signal sequences can only be expressed in a host cell of the same species (i.e., an insect cell signal sequence is optimally expressed in an insect cell). In contrast, other signal sequences can be used in heterologous hosts such as, for example, the human serum albumin (hHSA) signal sequence which works well in yeast, insect, or mammalian host cells and the tissue plasminogen activator pre/pro sequence which has been demonstrated to be functional in insect and mammalian cells (Tan et al., (2002) Protein Eng. 15:337). The choice of expression host can be made based on these and other factors, such as regulatory and safety considerations, production costs and the need and methods for purification. Thus, the vector system must be compatible with the host cell used.

Expression in eukaryotic hosts can include expression in yeasts such as Saccharomyces cerevisiae and Pichia pastoris, insect cells such as Drosophila cells and lepidopteran cells, plants and plant cells such as tobacco, corn, rice, algae, and Lemna. Eukaryotic cells for expression also include mammalian cells lines such as Chinese hamster ovary (CHO) cells or baby hamster kidney (BHK) cells. Eukaryotic expression hosts also include production in transgenic animals, for example, including production in serum, milk and eggs.

Recombinant molecules can be introduced into host cells via, for example, transformation, transfection, infection, electroporation and sonoporation, so that many copies of the gene sequence are generated. Generally, standard transfection methods are used to produce bacterial, mammalian, yeast, or insect cell lines that express large quantity of antibody chains, which are then purified using standard techniques (see e.g., Colley et al. (1989) J. Biol. Chem., 264:17619-17622; Guide to Protein Purification, in Methods in Enzymology, vol. 182 (Deutscher, ed.), 1990). Transformation of eukaryotic and prokaryotic cells is performed according to standard techniques (see, e.g., Morrison (1977) J. Bact. 132:349-351; Clark-Curtiss and Curtiss (1983) Methods in Enzymology, 101, 347-362). For example, any of the well-known procedures for introducing foreign nucleotide sequences into host cells can be used. These include the use of calcium phosphate transfection, polybrene, protoplast fusion, electroporation, biolistics, liposomes, microinjection, plasma vectors, viral vectors and any other the other well-known methods for introducing cloned genomic DNA, cDNA, synthetic DNA or other foreign genetic material into a host cell. Generally, for purposes of expressing an antibody, host cells are transfected with a first vector encoding at least a V_{H} chain and a second vector encoding at least a V_{L} chain. Thus, it is only necessary that the particular genetic engineering procedure used be capable of successfully introducing at least both genes into the host cell capable of expressing antibody polypeptide, or modified form thereof.

The modified anti-EGFR antibodies, provided herein, can be produced by any method known in the art for protein production including in vitro and in vivo methods such as, for example, the introduction of nucleic acid molecules encoding antibodies into a host cell or host animal and expression from nucleic acid molecules encoding recombined antibodies in vitro. Prokaryotes, especially E. coli, provide a system for producing large amounts of reassembled antibodies or portions thereof, and are particularly desired in applications of expression and purification of proteins. Transformation of E. coli is a simple and rapid technique well-known to those of skill in the art. E. coli host strains for high throughput expression include, but are not limited to, BL21 (EMD Biosciences) and LMG194 (ATCC). A particular example of such an E. coli host strain is BL21. Vectors for high throughput expression include, but are not limited to, pBR322 and pUC vectors.

### i. Prokaryotic Expression

Prokaryotes, especially E. coli, provide a system for producing large amounts of modified anti-EGFR antibodies, or portions thereof. Transformation of E. coli is a simple and rapid technique well-known to those of skill in the art. Expression vectors for E. coli can contain inducible promoters that are useful for inducing high levels of protein expression and for expressing antibodies that exhibit some toxicity to the host cells. Examples of inducible promoters include the lac promoter, the trp promoter, the hybrid tac promoter, the T7 and SP6 RNA promoters and the temperature regulated λP_{L} promoter.

Antibodies or portions thereof can be expressed in the cytoplasmic environment of E. coli. The cytoplasm is a reducing environment and for some antibodies, this can result in the formation of insoluble inclusion bodies. Reducing agents such as dithiothreitol and β-mercaptoethanol and denaturants (e.g., such as guanidine-HCl and urea) can be used to resolubilize the antibodies. An exemplary alternative approach is the expression of recombined antibodies or fragments thereof in the periplasmic space of bacteria which provides an oxidizing environment and chaperonin-like and disulfide isomerases leading to the production of soluble protein. Typically, a leader sequence is fused to the protein to be expressed which directs the protein to the periplasm. The leader is then removed by signal peptidases inside the periplasm. Exemplary pathways to translocate expressed proteins into the periplasm are the Sec pathway, the SRP pathway and the TAT pathway. Examples of periplasmic-targeting leader sequences include the pelB leader from the pectate lyase gene, the StII leader sequence, and the DsbA leader sequence. In some cases, periplasmic expression allows leakage of the expressed protein into the culture medium. The secretion of antibodies allows quick and simple purification from the culture supernatant. Antibodies that are not secreted can be obtained from the periplasm by osmotic lysis. Similar to cytoplasmic expression, in some cases proteins can become insoluble and denaturants and reducing agents can be used to facilitate solubilization and refolding using standard procedures. Temperature of induction and growth also can influence expression levels and solubility. Typically, temperatures between 25° C. and 37° C. are used. Mutations also can be used to increase solubility of expressed proteins. Typically, bacteria produce aglycosylated proteins. Thus, glycosylation can be added in vitro after purification from host cells.

### ii. Yeast

Yeasts such as Saccharomyces cerevisiae, Schizosaccharomyces pombe, Yarrowia lipolytica, Kluyveromyces lactis, and Pichia pastoris are useful expression hosts for recombined antibodies or portions thereof. Yeast can be transformed with episomal replicating vectors or by stable chromosomal integration by homologous recombination. Typically, inducible promoters are used to regulate gene expression. Examples of such promoters include AOX1, GAL1, GAL7, and GAL5 and metallothionein promoters such as CUP1. Expression vectors often include a selectable marker such as LEU2, TRP1, HIS3, and URA3 for selection and maintenance of the transformed DNA. Proteins expressed in yeast are often soluble. Co-expression with chaperonins such as BiP and protein disulfide isomerase can improve expression levels and solubility. Additionally, proteins expressed in yeast can be directed for secretion using secretion signal peptide fusions such as the yeast mating type alpha-factor secretion signal from Saccharomyces cerevisae and fusions with yeast cell surface proteins such as the Aga2p mating adhesion receptor or the Arxula adeninivorans glucoamylase. A protease cleavage site such as for the Kex-2 protease, can be engineered to remove the fused sequences from the expressed polypeptides as they exit the secretion pathway. Yeast also is capable of glycosylation at Asn-X-Ser/Thr motifs.

### iii. Insects

Insect cells, particularly using baculovirus expression, are useful for expressing modified anti-EGFR antibodies or portions thereof. Insect cells express high levels of protein and are capable of most of the post-translational modifications used by higher eukaryotes. Baculovirus have a restrictive host range which can improve the safety and reduce regulatory concerns of eukaryotic expression. Typical expression vectors use a promoter for high level expression such as the polyhedrin promoter and p10 promoter of baculovirus. Commonly used baculovirus systems include the baculoviruses such as Autographa californica nuclear polyhedrosis virus (AcNPV), and the Bombyx mori nuclear polyhedrosis virus (BmNPV) and an insect cell line such as Sf9 derived from Spodoptera frugiperda and TN derived from Trichoplusia ni. For high-level expression, the nucleotide sequence of the molecule to be expressed can be fused immediately downstream of the polyhedrin initiation codon of the virus. To generate baculovirus recombinants capable of expressing human antibodies, a dual-expression transfer, such as pAcUW51 (PharMingen) is utilized. Mammalian secretion signals are accurately processed in insect cells and can be used to secrete the expressed protein into the culture medium.

An alternative expression system in insect cell for expression of the modified anti-EGFR antibodies provided herein is the use of stably transformed cells. Cell lines such as Sf9 derived cells from Spodoptera frugiperda and TN derived cells from Trichoplusia ni can be used for expression. The baculovirus immediate early gene promoter IE1 can be used to induce consistent levels of expression. Typical expression vectors include the pIE1-3 and pI31-4 transfer vectors (Novagen). Expression vectors are typically maintained by the use of selectable markers such as neomycin and hygromycin.

### iv. Mammalian Cells

Mammalian expression systems can be used to express anti-EGFR antibodies, such as modified anti-EGFR antibodies, including antigen-binding fragments thereof. Expression constructs can be transferred to mammalian cells by viral infection such as adenovirus or by direct DNA transfer such as by using liposomes, calcium phosphate, DEAE-dextran and by physical means such as electroporation and microinjection. Expression vectors for mammalian cells typically include an mRNA cap site, a TATA box, a translational initiation sequence (Kozak consensus sequence) and polyadenylation elements. Such vectors often include transcriptional promoter-enhancers for high-level expression, for example the SV40 promoter-enhancer, the human cytomegalovirus (CMV) promoter and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha fetoprotein, alpha 1 antitrypsin, beta globin, myelin basic protein, myosin light chain 2, and gonadotropic releasing hormone gene control.

Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase and thymidine kinase. Modified anti-EGFR antibodies can be produced, for example, using a NEO^{R}/G418 system, a dihydrofolate reductase (DHFR) system or a glutamine synthetase (GS) system. The GS system uses joint expression vectors, such as pEE12/pEE6, to express both heavy chain and light chain. Fusion with cell surface signaling molecules such as TCR-ζ and FI_{ε}RI-γ can direct expression of the proteins in an active state on the cell surface.

Many cell lines are available for mammalian expression including mouse, rat, human, monkey, chicken and hamster cells. Exemplary cell lines include any known in the art or described herein, such as, for example, CHO, Balb/3T3, HeLa, MT2, mouse NSO (nonsecreting) and other myeloma cell lines, hybridoma and heterohybridoma cell lines, lymphocytes, fibroblasts, Sp2/0, COS, NIH3T3, HEK293, 293S, 2B8, and HKB cells. Cell lines adapted to serum-free media which facilitates purification of secreted proteins from the cell culture media also are available. One such example is the serum free EBNA-1 cell line (Pham et al., (2003) Biotechnol. Bioeng. 84:332-42.)

### v. Plants

Transgenic plant cells and plants can be used to express anti-EGFR antibodies, such as modified anti-EGFR antibodies, or a portion thereof described herein. Expression constructs are typically transferred to plants using direct DNA transfer such as microprojectile bombardment and PEG-mediated transfer into protoplasts, and with agrobacterium-mediated transformation. Expression vectors can include promoter and enhancer sequences, transcriptional termination elements and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus CaMV 35S promoter, the nopaline synthase promoter, the ribose bisphosphate carboxylase promoter and the maize ubiquitin-1 (ubi-1) promoter promoters. Selectable markers such as hygromycin, phosphomannose isomerase and neomycin phosphotransferase are often used to facilitate selection and maintenance of transformed cells. Transformed plant cells can be maintained in culture as cells, aggregates (callus tissue) or regenerated into whole plants. Transgenic plant cells also can include algae engineered to produce proteases or modified proteases (see for example, Mayfield et al. (2003) PNAS 100:438-442). Because plants have different glycosylation patterns than mammalian cells, this can influence the choice of protein produced in these hosts.

2. Purification

Anti-EGFR antibodies, such as modified anti-EGFR antibodies and antigen binding portions thereof, can be purified by any procedure known to one of skill in the art or described herein. Proteins can be purified to substantial purity using standard protein purification techniques known in the art including but not limited to, SDS-PAGE, size fraction and size exclusion chromatography, ammonium sulfate precipitation, chelate chromatography, ionic exchange chromatography or column chromatography. For example, antibodies can be purified by column chromatography. An exemplary method for purifying the anti-EGFR antibodies provided herein is column chromatography, wherein a solid support column material is linked to Protein G, a cell surface-associated protein from Streptococcus, that binds immunoglobulins with high affinity. In some examples, the anti-EGFR antibodies can be purified by column chromatography, wherein a solid support column material is linked to Protein A, a cell surface-associated protein from Staphylococcus that binds immunoglobulins, such as IgG antibodies, with high affinity (see, e.g., Liu et al. (2010) MAbs 2(5):480-499). Other immunoglobulin-binding bacterial proteins that can be used to purify the anti-EGFR antibodies provided herein include Protein A/G, a recombinant fusion protein that combines the IgG binding domains of Protein A and Protein G; and Protein L, a surface protein from Peptostreptococcus (Bjorck (1988) J. Immunol., 140(4):1194-1197; Kastern, et al. (1992) J. Biol. Chem. 267(18):12820-12825; Eliasson et al. (1988) J. Biol. Chem. 263:4323-4327).

The anti-EGFR antibodies can be purified to 60%, 70%, 80% purity and typically at least 90%, 91%, 92%, 93%, 94%, 95%, 96%, 97%, 98% or 99% purity. Purity can be assessed by standard methods such as by SDS-PAGE and coomassie staining.

Methods for purification of anti-EGFR antibodies, including antibodies or portions thereof from host cells, depend on the chosen host cells and expression systems. For secreted molecules, proteins are generally purified from the culture media after removing the cells. For intracellular expression, cells can be lysed and the proteins purified from the extract. When transgenic organisms such as transgenic plants and animals are used for expression, tissues or organs can be used as starting material to make a lysed cell extract. Additionally, transgenic animal production can include the production of polypeptides in milk or eggs, which can be collected, and if necessary further the proteins can be extracted and further purified using standard methods in the art.

When proteins are expressed by transformed bacteria in large amounts, typically after promoter induction, although expression can be constitutive, the polypeptides can form insoluble aggregates. There are several protocols that are suitable for purification of polypeptide inclusion bodies known to one of skill in the art. Numerous variations will be apparent to those of skill in the art.

### E. METHODS FOR ASSESSING ANTI-EGFR ANTIBODY PROPERTIES AND ACTIVITIES

The modified anti-EGFR antibodies, and variants and fragments thereof, provided herein, can be assessed for binding to EGFR antigen (e.g., human EGFR) or soluble fragment thereof. The binding activity can be assessed under conditions that compare the activity of an antibody under conditions of low pH/increased lactate concentrations and neutral pH/physiological lactate concentrations. For example, binding activity can be assessed under conditions of acidic pH (e.g., pH 5.8 to 6.5, such as pH 6.0 to 6.5) and/or increased lactate concentrations (e.g., 10 to 20 mM, such as 15 to 20 mM) and compared to binding activity under conditions of neutral pH (e.g., pH 7.0 to 7.6, such as pH 7.0 to 7.4) and/or physiological lactate concentrations (e.g., 0.5 to 5 mM, such as about 1 mM). Such assays can confirm that the binding activity is greater under conditions that include one or both of acid pH 6.0 to 6.5, inclusive, and/or lactate concentration of 15 mM to 20 mM, inclusive, compared to under conditions that include one or both of neutral pH of or about 7.4 and/or lactate concentration of or about 1 mM.

The assays also can be performed in the presence of physiological concentrations of protein or serum (e.g., 10 mg/mL to 50 mg/mL protein, such as serum albumin; or 20-50% serum, such as human serum). For example, the anti-EGFR antibodies typically are assessed for activity under a first set of conditions that includes 20-50% serum (vol/vol) or 10-50 mg/mL protein (e.g., serum albumin), and an acidic pH of about between 5.8 to 6.8 and/or elevated lactate levels of 10 mM to 20 mM. For example, the first set of conditions can include at least 25% serum (vol/vol) or 12-40 mg/mL protein (e.g., serum albumin), and an acidic pH of about between 6.0 to 6.5, such as pH 6.0 and/or elevated lactate levels of 15 mM to 20 mM, such as about 16.7 mM. The anti-EGFR antibody also is assessed for activity under a second set of conditions that includes 20-50% serum (vol/vol) or 10-50 mg/mL protein (e.g., serum albumin), and near neutral pH or neutral pH of about between 7.0 to 7.4 and/or a lactate concentration of 0.5 to 5 mM. For example, the second set of conditions includes at least 25% serum (vol/vol) or 12-40 mg/mL protein (e.g., serum albumin), and pH about between 7.2 to 7.4, such as about pH 7.4. and/or lactate concentration of 0.5 mM to 2 mM, such as 1 mM. In some examples, the anti-EGFR antibody also can be assessed for activity under a third set of conditions that can include at least 25% serum (vol/vol) or 12-40 mg/mL protein (e.g., serum albumin), and an acidic pH of about between 6.0 to 6.5, such as pH 6.5 and/or elevated lactate levels of 15 mM to 20 mM, such as about 16.7 mM. In such assays, the amount of added protein to simulate a physiologic environment (e.g., serum protein) is typically the same or substantially the same for all sets of conditions tested, but can vary by ±25% or less from one condition to the other.

Hence, binding activity can be determined in conditions that simulate a physiologic environment or that is a physiologic environment. The binding can be assessed in vitro (e.g., in an immunoassay) or ex vivo or in vivo (e.g., binding to tumor cells or non-tumor cells, such as cells of the skin dermis). The assays provided herein include any assays that can test or assess an activity of a modified anti-EGFR antibody in a detectable or otherwise measurable manner under different pH and/or lactate concentrations, and, optionally, in the presence of physiological concentrations of total protein. The assays provided herein can be developed in a high throughput format in order to assess an activity of numerous anti-EGFR antibodies, for example protein variants, at one time in dual format. For example, in vitro binding assays can be performed using solid-support binding assays or solution binding assays, where the binding is performed under the above conditions. In other examples, binding assays can be performed in vivo where binding is compared on cells present in a tumor versus cells present in non-tumor cells. In particular, an in vivo binding assay can be performed to assess binding or localization of administered antibody to tumor cells versus basal skin keratinocytes. This is exemplified herein using xenograft or skin graft models. Other models also can be employed. Descriptions of exemplary assays are provided below.

In addition to binding activity, other assays to assess the activity of modified anti-EGFR antibodies provided herein can be performed and include in vitro or in vivo assays including, but not limited to, functional assays, in vivo assays, animal models and clinical assays to measure the activity and/or side effects of the modified anti-EGFR antibodies provided herein. The activity assessed can be any activity of an anti-EGFR antibody, such as binding to EGFR, cell growth inhibition (CGI) activity or tumor growth inhibition activity. Any of the antibodies provided herein also can be characterized in a variety of assays known to one of skill in the art to assess clinical properties such as, for example, therapeutic efficacy, affinity for EGFR, toxicity, side effects, pharmacokinetics and pharmacodynamics.

1. Binding Assays

Modified anti-EGFR antibodies can be assayed for the ability to bind to EGFR by any method known to one of skill in the art. Exemplary assays are described herein below.

Binding assays can be performed in solution, suspension or on a solid support. For example, EGFR or soluble fragment thereof can be immobilized to a solid support (e.g., a carbon or plastic surface, a tissue culture dish or chip) and contacted with antibody. Unbound antibody or target protein can be washed away and bound complexes can then be detected. Binding assays can be performed under conditions to reduce nonspecific binding, such as by using buffers with a high ionic strength (e.g., 0.3-0.4 M NaCl) and/or with nonionic detergent (e.g., 0.1% Triton X-100 or Tween 20) and/or blocking proteins (e.g., bovine serum albumin or gelatin). Negative controls also can be included in such assays as a measure of background binding. Binding affinities can be determined using quantitative ELISA, Scatchard analysis (Munson et al., (1980) Anal. Biochem., 107:220), surface plasmon resonance, isothermal calorimetry, or other methods known to one of skill in the art (e.g., Liliom et al. (1991) J. Immunol Methods. 143(1):119-25).

Such assays can be performed, for example, in solution (e.g., Houghten (1992) Bio/Techniques 13:412-421), on beads (Lam (1991) Nature 354:82-84), on chips (Fodor (1993) Nature 364:555-556), on bacteria (U.S. Pat. No. 5,223,409), on spores (U.S. Pat. Nos. 5,571,698; 5,403,484; and 5,223,409), on plasmids (Cull et al. (1992) Proc. Natl. Acad. Sci. USA 89:1865-1869) or on phage (Scott and Smith (1990) Science 249:386-390; Devlin (1990) Science 249:404-406; Cwirla et al. (1990) Proc. Natl. Acad. Sci. USA 87:6378-6382; and Felici (1991) J Mol. Biol. 222:301-310).

Typically, EGFR binding is detected using a method that is capable of being quantified such that the level of activity can be assessed. For example, methods of quantitation include, but are not limited to, spectrophotometric, fluorescent and radioactive methods. Such methods measure, for example, colorimetric signals, chemiluminescent signals, chemifluorescent signals or radioactive signals.

In some examples, the modified anti-EGFR antibodies provided herein can be labeled with a detectable moiety or tag to facilitate detection and determination of EGFR biding activity. The skilled artisan can select an appropriate detectable moiety or tag for use in the assays described or known in the art. Any detectable moiety (i.e., tag or other moiety known to one of skill in the art) that is capable of being detected or identified can be linked to the modified anti-EGFR antibody or fragment to be tested, directly or indirectly, for example using a linker. Linkage can be at the N- or C-terminus of the therapeutic antibody. Exemplary tags and moieties are set forth in Table 13.

TABLE 13
Exemplary tags and moieties

| | | | | SEQ |
|---|---|---|---|---|
| | | # of | Size | ID |
| Name | Sequence | Residues | (Da) | NO |
| c-Myc | EQKLISEEDL | 10 | 1200 | 335 |
| FLAG | DYKDDDDK | 8 | 1012 | 45 |
| His | HHHHHH | 6 | | 44 |
| HA | YPYDVPDYA | 9 | 1102 | 336 |
| VSV-G | YTDIEMNRLGK | 11 | 1339 | 337 |
| HSV | QPELAPEDPED | 11 | 1239 | 338 |
| V5 | GKPIPNPLLGLDST | 14 | 1421 | 339 |
| Poly Arg | RRRRR | 5-6 | 800 | 340 |
| Strep-tag-II | WSHPQFEK | 8 | 1200 | 341 |
| S | KETAAAKFERQHMDS | 15 | 1750 | 342 |
| 3x FLAG | DYKDHDGDYKDHDIDYKDDD | 22 | 2730 | 343 |
| | DK | | | |
| HAT | KDHLIHNVHKEFHAHAHNK | 19 | 2310 | 344 |
| SBP | MDEKTTGWRGGHVVEGLAGEL | 38 | 4306 | 345 |
| | EQLRARLEHHPQGQREP | | | |

Any linker known to one of skill in the art that is capable of linking the detectable moiety to the therapeutic antibodies described herein can be used. Exemplary linkers include the glycine rich flexible linkers (-G₄S—)ₙ, where n is a positive integer, such as 1 (SEQ ID NO: 346), 2 (SEQ ID NO: 347), 3 (SEQ ID NO: 46), 4 (SEQ ID NO: 348), 5 (SEQ ID NO: 349), or more.

Binding assays can be performed in solution, by affixing the modified anti-EGFR antibody to a solid support, or by affixing EGFR to a solid support. Any solid support binding assay known to the skilled artisan is contemplated for testing the activities of the antibodies provided herein, including, but not limited to, surface plasmon resonance, bio-layer interferometry, immunoassays, binding to tissues using immunofluorescence or immunohistochemistry, solution binding assays, and cell based binding assays (e.g., using any of the EGFR-expressing cells described below).

Immunoassays include competitive and non-competitive assay systems using techniques such as, but not limited to, western blots or immunoblots, such as quantitative western blots; radioimmunoassays; ELISA (enzyme linked immunosorbent assay); Meso

Scale Discovery (MSD, Gaithersburg, Md.); “sandwich” immunoassays; immunoprecipitation assays; ELISPOT; precipitin reactions; gel diffusion precipitin reactions; immunodiffusion assays; agglutination assays; complement-fixation assays; immunoradiometric assays; fluorescent immunoassays; protein A immunoassays; immunohistochemistry; immuno-electron microscopy or liposome immunoassays (LIA). Such assays are routine and well-known in the art (see, e.g., Ausubel et al., Eds, 1994, Current Protocols in Molecular Biology, Vol. 1, John Wiley & Sons, Inc., New York).

In some examples, immunohistochemistry and/or immunofluorescence can be used to assess EGFR binding, ex vivo, in animal models. For example, antibody binding to xenograft tumors in a rodent or other animal model can be analyzed. In other examples, immunohistochemistry can be used to assess modified anti-EGFR antibody binding to skin, such as primate skin. In other examples, immunohistochemistry can be used to assess binding to xenograft tumors and primate skin grafts, ex vivo, for example to visually or quantitatively compare binding preferences of the antibody and to determine if the tested antibody exhibits selective or conditional binding in vivo (see, e.g., Examples 11-13).

In other examples, an animal model containing a xenograft tumor or skin graft, such as an animal model described herein, can be administered a modified anti-EGFR antibody provided herein, such as by systemic administration., to assess in vivo binding of the modified anti-EGFR antibody. In such examples, the tissue can be harvested at particular time(s) to assess binding ex vivo by immunohistochemistry or immunofluorescence as described above. In other examples, the administered modified anti-EGFR antibody is conjugated to a fluorophore, such as an infrared fluorophore (e.g., DyLight⁷⁵⁵), which is capable of transmitting fluorescence through the skin. In such examples, anti-EGFR antibody binding can be visualized in vivo using a fluorescent imaging system such as the IVIS Caliper imaging system, and antibody binding to xenograft tumors and/or primate skin grafts can be assessed (see, e.g., Example 13). Tissue can subsequently be harvested for ex vivo confirmational immunohistochemical analysis.

Solution binding assays, including any solution binding assay known to the skilled artisan, can be used to assess binding activity including equilibrium dialysis, competitive binding assays (e.g., Myers et al., (1975) Proc. Natl. Acad. Sci. USA), radiolabeled binding assays (e.g., Feau et al., (2009) J. Biomol. Screen. 14(1):43-48), calorimetry, including isothermal titration calorimetry (ITC) and differential scanning calorimetry (e.g., Alvarenga et al. (2012) Anal. Biochem 421(1):138-151, Perozzo et al., (2004) J. Recept Signal. Transduct Res. 24(1-2):1-52; Holdgate (2001) Biotechniques 31(1):164-166, 168, 170, Celej et al. (2006) Anal. Biochem. 350(2):277-284), and spectroscopic fluorescence assays, including fluorescence resonance energy transfer (FRET) assays (Wu et al. (2007), J. Pharm. Biomed. Anal. 44(3):796-801). The conditions for binding assays in can be adapted from conditions discussed above for binding assays performed on a solid support.

Depending on the quantitative assay selected to measure antibody binding, absolute binding can be represented, for example, in terms of optical density (OD), such as from densitometry or spectrophotometry measurements; arbitrary fluorescent units (AFU), such as from fluorescence measurements; or lumens, such as from chemiluminescence measurements. In some examples, the specific activity is calculated by dividing the absolute binding signal by the antibody protein concentration. In some examples, the specific activity is normalized to give a normalized specific activity (NSA) for each modified anti-EGFR antibody by dividing the specific activity of the modified anti-EGFR antibody by the specific activity of a reference antibody, such as an unmodified anti-EGFR parental antibody, such as wild-type Cetuximab.

Binding activity also can be measured in terms of binding affinity, which can be determined in terms of binding kinetics, such as measuring rates of association (kₐ or kₒₙ) and/or dissociation (k_{d} or k_{off}), half maximal effective concentration (EC₅₀) values, and/or thermodynamic data (e.g., Gibbs free energy (ΔG), enthalpy (ΔH), entropy (−TΔS), and/or calculating association (K_{A}) or dissociation (K_{D}) constants. Typically, determination of binding kinetics requires known antibody and EGFR protein concentrations. Rates of association (kₐ) and association constants (K_{A}) are positively correlated with binding affinity. In contrast, rates of dissociation (k_{d}), dissociation constants (K_{D}) and EC₅₀ values are negatively correlated with binding affinity. Thus, higher binding affinity is represented by lower k_{d}, K_{D} and EC₅₀ values.

Comparing the binding activities of the modified antibodies provided herein under conditions of low pH and/or elevated lactate levels and conditions of neutral pH and/or normal lactate levels can be accomplished by comparing absolute binding under identical conditions (e.g., identical protein dilutions and binding conditions, other than the different pH and/or lactate conditions) or by comparing binding affinity under the different conditions.

Thus, using such assays, the ratio of binding activity under conditions that include one or both of pH 6.0 to 6.5, inclusive, and/or lactate concentration of between 10 mM to 20 mM, inclusive, compared to under conditions of one or both of neutral pH of or about 7.4 and/or lactate concentration of or about 1 mM can be determined Depending on the assay employed, the binding activity can be a ratio of a quantified value that is an absolute value (e.g., optical density), a concentration measurement of binding or potency (e.g., EC50) or a kinetic measurement (e.g., association or dissociation constant). For purposes herein, in all instances, the ratio is determined in a manner such that a ratio of greater than 1 indicates binding is greater (e.g., tighter binding affinity or lower EC₅₀) under conditions that include one or both of pH 6.0 to 6.5, inclusive, and/or lactate concentration of between 10 mM to 20 mM, inclusive, compared to under conditions of one or both of neutral pH of or about 7.4 and/or lactate concentration of or about 1 mM.

For example, in examples where absolute binding is measured, conditional binding can be determined by calculating the ratio of absolute binding under acidic pH (e.g., pH 6.0 or 6.5) and/or elevated lactate (e.g., 16.7 mM) conditions versus the absolute binding under neutral pH (e.g., pH 7.4) and/or normal lactate (e.g., 1 mM) conditions, for example, by determining the quotient of OD_{pH 6.0}/OD_{pH 7.4} or OD_{pH 6.5}/OD_{pH 7.4}. When binding activity is determined in terms of kinetic measures that are positively correlated with binding affinity (e.g., kₐ and K_{A}), the ratio of activity can be evaluated by calculating the ratios of positively correlating terms under acidic pH (e.g., pH 6.0 or 6.5) and/or elevated lactate (e.g., 16.7 mM) conditions versus the absolute binding under neutral pH (e.g., pH 7.4) and/or normal lactate (e.g., 1 mM) conditions, for example, by determining the quotient of K_{A(pH 6.0)}/K_{A(pH 7.4)} or K_{A(pH 6.5)}/K_{A(pH 7.4)}. When binding activity is determined in terms of binding measures that are negatively or inversely correlated with binding affinity (e.g., K_{D} or EC₅₀), ratio of binding activity can be evaluated by calculating the ratios of the inverse, or reciprocal, of the negatively correlating terms under acidic pH (e.g., pH 6.0 or 6.5) and/or elevated lactate (e.g., 16.7 mM) conditions versus the absolute binding under neutral pH (e.g., pH 7.4) and/or normal lactate (e.g., 1 mM) conditions, for example, by determining the quotient of (1/EC_{50 (pH 6.0)}(1/EC_{50 (pH 7.4)}.

Typically, antibodies provided herein have a ratio of binding activity that is at least 2, 3, 4, 5, 6, 7, 8, 9, 10, 15, 20, 25, 30, 35, 40 or 50, indicating at least a 2-fold, 3-fold, 4-fold, 5-fold, 6-fold, 7-fold, 8-fold, 10-fold, 15-fold, 20-fold, 25-fold, 30-fold, 35-fold, 40-fold, 50-fold or more greater binding (e.g., tighter binding affinity or lower EC₅₀).

### 2. Other Cell Based Functional Assays

Assays to measure activity of the anti-EGFR antibody, such as a modified anti-EGFR antibodies, provided herein include cell based assays. Cell lines that can be used include any cell lines described in the art or cell lines that can be obtained from repositories such as the American Type Culture Collection (ATCC). The skilled artisan can select cell lines with desired properties. Generally, assays are performed using cell lines known to express EGFR. Such cells are known to one of skill in the art. For example, one can consult the ATCC Catalog (atcc.org) to identify cell lines.

Exemplary cell lines that express EGFR that can be used in cell based assays to screen the anti-EGFR antibodies provided herein include DiFi human colorectal carcinoma cells, A431 cells (ATCC CRL-1555), Caco-2 colorectal adenocarcinoma cells (ATCC HTB-37), HRT-18 colorectal adenocarcinoma cells (ATCC CCL-244), HT-29 colorectal adenocarcinoma cells (ATCC HTB-38), human neonatal keratinocytes and MCF10A epithelial cells (ATCC CRL-10317) (see, e.g., Olive et al. (1993) In Vitro Cell Dev Biol. 29A(3 Pt 1):239-248; Wu et al. (1995) J. Clin. Invest. 95(4): 1897-1905). Exemplary cells that can be used in the cell based assays described herein include any cells described herein or known in the art, including, for example, tumor or cancer cells described herein.

In some examples, assays to measure the activity of an anti-EGFR antibody, such as modified anti-EGFR antibodies provided herein, such as the assays described herein, are performed using cell lines from a tissue associated with a side effect of anti-EGFR antibodies, such as any side effect described herein or known in the art. For example, assays can be performed using skin cell lines. EGFR is expressed in several cell types, including keratinocytes, such as basal keratinocytes and the outer root sheath of hair follicles; and cells of eccrine and sebaceous glands (Albanell et al. (2002) J. Clin. Oncol. 20(1):110-124; Lacouture, and Melosky (2007) Skin Therapy Lett. 12, 1-5; Nanney et al. (1990) J. Invest. Dermatol 94(6):742-748).

In some examples, cell-based assays to measure activity of the anti-EGFR antibodies provided herein are performed using keratinocytes, such as, for example, human neonatal keratinocytes; cells from the outer root sheath of hair follicles; and cells of eccrine and sebaceous glands. Other cells that can be used in cell-based assays to measure activity of the anti-EGFR antibodies provided herein include, for example, melanocytes, such as, for example, newborn melanocytes; Langerhans cells; fibroblasts; Merkel's cells; nerve cells; glandular cells; sebaceous gland cells (sebocytes); and fibroblasts, such as, for example dermal fibroblasts and wound fibroblasts. Methods of culturing such cells are within the ability of the skilled artisan (see, e.g., Limat and Hunziker (1996) Methods Mol Med. 2:21-31; Abdel-Naser et al. (2005) Egypt. Dermatol. Online J. 1(2):1).

Cell lines expressing EGFR can be generated by transient or stable transfection. In addition, any primary cell or cell line can be assessed for expression of EGFR, such as by using fluorescently labeled anti-EGFR antibodies and fluorescence activated cell sorting (FACS). Exemplary cell lines include A549 (lung), HeLa, Jurkat, BJAB, Colo205, H1299, MCF7, MDA-MB-231, PC3, HUMEC, HUVEC, and PrEC.

Activity of the modified anti-EGFR antibodies provided herein, can be assessed, for example, using any assay that can detect the binding to the surface of the cells. Activity also can be assessed by assessing a functional activity of the anti-EGFR antibodies. In some examples, the assays are based on the biology of the ability of the anti-EGFR antibody to bind to EGFR and mediate some biochemical event, for example, effector functions like cellular lysis, phagocytosis, ligand/receptor binding inhibition, inhibition of growth and/or proliferation and apoptosis.

Such assays often involve monitoring the response of cells to a modified anti-EGFR antibody, for example cell survival, cell death, cellular phagocytosis, cell lysis, change in cellular morphology, or transcriptional activation such as cellular expression of a natural gene or reporter gene. For example, cell proliferation assays, cell death assays, flow cytometry, cell separation techniques, fluorescence activated cell sorting (FACS), phase microscopy, fluorescence microscopy, receptor binding assays, cell signaling assays, immunocytochemistry, reporter gene assays, cellular morphology (e.g., cell volume, nuclear volume, cell perimeter, and nuclear perimeter), ligand binding, substrate binding, nuclease activity, apoptosis, chemotaxis or cell migrations, cell surface marker expression, cellular proliferation, GFP positivity and dye dilution assays (e.g., cell tracker assays with dyes that bind to cell membranes), DNA synthesis assays (e.g., 3H-thymidine and fluorescent DNA-binding dyes such as BrdU or Hoechst dye with FACS analysis) and nuclear foci assays, are all suitable assays to measure the activity of the modified anti-EGFR antibodies provided herein. Other functional activities that can be measured include, but are not limited to, ligand binding, substrate binding, endonuclease and/or exonuclease activity, transcriptional changes to both known and uncharacterized genetic markers (e.g., northern blots), changes in cell metabolism, changes related to cellular proliferation, cell surface marker expression, DNA synthesis, marker and dye dilution assays (e.g., GFP and cell tracker assays), contact inhibition, tumor growth in nude mice, and others.

For example, modified anti-EGFR antibodies provided herein can be assessed for their modulation of one or more phenotypes of a cell known to express EGFR. Phenotypic assays, kits and reagents for their use are well-known to those skilled in the art and are herein used to measure the activity of modified anti-EGFR antibodies. Representative phenotypic assays, which can be purchased from any one of several commercial vendors, include those for determining cell viability, cytotoxicity, proliferation or cell survival (Molecular Probes, Eugene, Oreg.; PerkinElmer, Boston, Mass.), protein-based assays including enzymatic assays (Panvera, LLC, Madison, Wis.; BD Biosciences, Franklin Lakes, N.J.; Oncogene Research Products, San Diego, Calif.), cell regulation, signal transduction, inflammation, oxidative processes and apoptosis (Assay Designs Inc., Ann Arbor, Mich.), triglyceride accumulation (Sigma-Aldrich, St. Louis, Mo.), angiogenesis assays, tube formation assays, cytokine and hormone assays and metabolic assays (Chemicon International Inc., Temecula, Calif.; Amersham Biosciences, Piscataway, N.J.).

Cells determined to be appropriate for a particular phenotypic assay (i.e., any cell described herein or known in the art to express EGFR) can be treated with an anti-EGFR antibody as well as control antibody. In some examples, EGF, or a fragment thereof, is included so that activation of the receptor is effected. At the end of the treatment period, treated and untreated cells can be analyzed by one or more methods described herein or known in the art. In some examples, activity of the anti-EGFR antibodies provided herein can be assessed by measuring changes in cell morphology, measuring EGFR phosphorylation or cell proliferation.

The assays can be performed to assess the effects of an anti-EGFR antibody, such as a modified anti-EGFR antibody, on EGFR and/or on cells that express EGFR. In some examples, the activity of EGFR can be stimulated in the presence of EGF or another stimulating agent in the presence or absence of the anti-EGFR antibody provided herein to determine if the antibody modulates (e.g., inhibits) the actions of EGF or another stimulating agent. For example, the anti-EGFR antibody can act by blocking the ability of EGF to interact with EGFR. Thus, the modified anti-EGFR antibodies provided herein also can be tested for antagonistic properties.

For example, EGFR phosphorylation assays can be used to measure the ability of the anti-EGFR antibodies provided herein to inhibit phosphorylation of EGFR. Binding of EGF to the extracellular domain of EGFR induces receptor dimerization, and tyrosine phosphorylation, and can result in uncontrolled proliferation (Seshacharyulu et al. (2012) Expert. Opin. Ther. Targets. 16(1):15-31). Modified anti-EGFR antibodies provided herein, can inhibit EGF binding to EGFR and decrease EGFR phosphorylation (see, e.g., U.S. Pat. No. 8,071,093). Thus, activity of an anti-EGFR antibody provided herein can be assessed by detecting phosphorylated EGFR. In some examples, phosphorylated EGFR can be detected in cell lysates by an ELISA assay using methods known in the art or described herein (see, e.g., Example 8). The dose-dependence of the modified anti-EGFR antibodies on the inhibitory effect can be determined by plotting the concentration of phosphorylated EGFR against the concentration of modified anti-EGFR antibody. Tyrosine phosphorylated forms of EGFR can be detected using EGFR Phospho ELISA kits available from, e.g., Sigma-Aldrich (St. Louis, Mo.), RAYBIO (Norcross, Ga.) or Thermo Scientific (Rockford, Ill.).

Growth assays can be used to measure the activity of the modified anti-EGFR antibodies. The assays can measure growth inhibition of cells that express EGFR by an anti-EGFR antibody, such as a modified anti-EGFR antibody. Cells can be incubated for a sufficient time for cells to grow (e.g., 12 hours, or 1, 2, 3, 4, 5, 6, 7 days or longer). Cell growth can be measured by any method known in the art, including ³H-thymidine incorporation assay, 5-bromo-2-deoxyuridine (BrdU) ELISA, tetrazolium microplate assay and acid phosphatase assay (e.g., Maghni et al. (1999) J. Immunol. Method. 223(2):185-194). Cell growth can also be measured using kits available from Invitrogen (Cyquant NF cell proliferation assay kit), Cambrex (ViaLight HS (high sensitivity) BioAssay), Promega (CellTiter-Glo Luminescent Cell Viability Assay, Guava Technologies (CellGrowth assay), Stratagene (Quantos cell proliferation assay) (e.g., Assays for Cell Proliferation Studies, Genetic Eng. Biotechnol. News. 26(6)). In some examples, the cell growth can be normalized to growth of cells without antibody. In exemplary growth assays, cells can be added to a well of a 96-well plate in normal growth medium that includes the anti-EGFR antibody to be assayed. An exemplary cell growth assay is described in Example 9.

3. Animal Models

In vivo studies using animal models also can be performed to assess the therapeutic activity of modified anti-EGFR antibodies provided herein. An anti-EGFR antibody can be administered to animal models of the diseases and conditions for which therapy using a modified anti-EGFR antibody provided herein is considered. Such animal models are known in the art, and include, but are not limited to, xenogenic cancer models wherein human cancer explants or passaged xenograft tissues are introduced into immune compromised animals, such as nude or SCID mice, (see e.g., Klein. et al. (1997) Nature Medicine 3:402-408). Efficacy can be predicted using assays that measure inhibition of tumor formation, tumor regression or metastasis Animal models also can be used to assess side effects of the anti-EGFR antibodies provided herein.

Various tumor cell lines or tumor animal models are known to one of skill in the art and are described herein. Activity of the anti-EGFR antibodies can be assessed by monitoring parameters indicative of treatment of a disease or condition that can be treated by administration of anti-EGFR antibodies. For example, an anti-EGFR antibody can be administered to a tumor-bearing animal followed by monitoring body weight and tumor volume. For example, a parameter indicative of anti-tumorigenicity is shrinkage of tumor size and/or delay in tumor progression. Hence, for example, anti-EGFR antibodies can be assessed to identify those that decrease tumor growth or size. Tumor size can be assessed in vivo in tumor-bearing human or animal models treated with an anti-EGFR antibody. Tumor shrinkage or tumor size can be assessed by various assays known in art, such as, by weight, volume or physical measurement. The anti-EGFR antibody also can be administered to normal animals, and body weights monitored to assess adverse side effects associate with administering the anti-EGFR antibody.

In vivo tumors can be generated in animals by any known method, including xenograft tumors generated by inoculating or implanting tumor cells (e.g., by subcutaneous injection) into an immunodeficient rodent, syngenic tumors models generated by inoculating (e.g., by subcutaneous injection) a mouse or rat tumor cell line into the corresponding immunocompetent mouse or rat strain, metastatic tumors generated by metastasis of a primary tumor implanted in the animal model, allograft tumors generated by the implantation of tumor cells into same species as the origin of the tumor cells, and spontaneous tumors generated by genetic manipulation of the animal. The tumor models can be generated orthotopically by injection of the tumor cells into the tissue or organ of their origin, for example, implantation of breast tumor cells into a mouse mammary fat pad. In some examples, xenograft models or syngenic models are used. For example, tumors can be established by subcutaneous injection at the right armpit with a tumor cell suspension (e.g., 1×10⁶ to 5×10⁶ cells/animal) into immunocompetent hosts (syngeneic) or immunodeficient hosts (e.g., nude or SCID mice; xenograft). The animal models include models in any organism described herein or known in the art, such as, for example, a mammal, including monkeys and mice.

The tumor can be syngeneic, allogeneic, or xenogeneic. The tumor can express endogenous or exogenous EGFR. Exogenous EGFR expression can be achieved using methods of recombinant expression known in the art or described herein via transfection or transduction of the cells with the appropriate nucleic acid. Exemplary cell lines include EGFR transfected NIH3T3, MCF7 (human mammary), human epidermoid squamous carcinoma A431, oral squamous cell carcinoma (OSCC) cell line BcaCD885, COLO 356/FG pancreatic cell lines, colorectal carcinoma cell lines, HT29 or LS174T, and MDA-MB-231 triple negative breast cancer cell lines (see e.g., Santon et al., (1986) Cancer Res. 46:4701-05 and Ozawa et al., (1987) Int. J. Cancer 40:706-10; U.S. Pat. Pub. No. 20110111059; Reusch et al. (2006) Clin. Cancer Res. 12(1):183-190; and Yang et al. (2011) Int. J. Nanomedicine 6:1739-1745).

The modified anti-EGFR antibodies provided herein can be tested in a variety of orthotopic tumor models. These animal models are used by the skilled artisan to study pathophysiology and therapy of aggressive cancers such as, for example, pancreatic, prostate and breast cancer Immune deprived mice including, but not limited to athymic nude or SCID mice can be used in scoring of local and systemic tumor spread from the site of intraorgan (e.g., pancreas, prostate or mammary gland) injection of human tumor cells or fragments of donor patients.

In some examples, the testing of anti-EGFR targeting proteins can include study of efficacy in primates (e.g., cynomolgus monkey model) to facilitate the evaluation of depletion of specific target cells harboring EGFR antigen. Additional primate models include but are not limited to that of the rhesus monkey.

For example, the recipient of the tumor can be any suitable murine strain. The recipient can be immunocompetent or immunocompromised in one or more immune-related functions, including but not limited to nu/nu, SCID, and beige mice. Examples of animals in which tumor cells can be transplanted include BALB/c mice, C57BL/6 mice, severe combined immunodeficient/Beige mice (SCID-Beige) (see, e.g., U.S. Pat. Pub. No. 20110111059; Reusch et al. (2006) Clin. Cancer Res. 12(1):183-190; Yang et al. (2011) Int. J. Nanomedicine 6:1739-1745). Other examples include nude mice, SCID mice, xenograft mice, and transgenic mice (including knockins and knockouts). For example, an anti-EGFR antibody provided herein can be tested in a mouse cancer model, for example a xenograft mouse. In this method, a tumor or tumor cell line is grafted onto or injected into a mouse, and subsequently the mouse is treated with an anti-EGFR antibody to determine the ability of the anti-EGFR antibody to reduce or inhibit cancer growth and metastasis. Also contemplated is the use of a SCID murine model in which immune-deficient mice are injected with human peripheral blood lymphocytes (PBLs).

Exemplary human tumor xenograft models in mice, such as nude or SCID mice, include, but are not limited to, human lung carcinoma (A549 cells, ATCC No. CCL-185); human breast tumor (GI-101A cells, Rathinavelu et al., (1999) Cancer Biochem. Biophys., 17:133-146 or MDA-MB-231 triple negative breast cancer cells); human ovarian carcinoma (OVCAR-3 cells, ATCC No. HTB-161); human pancreatic carcinoma (PANC-1cells, ATCC No. CRL-1469 and MIA PaCa-2 cells, ATCC No. CRL-1420); DU145 cells (human prostate cancer cells, ATCC No. HTB-81); human prostate cancer (PC-3 cells, ATCC# CRL-1435); colon carcinoma (HT-29 cells); human melanoma (888-MEL cells, 1858-MEL cells or 1936-MEL cells; see e.g., Wang et al., (2006) J. Invest. Dermatol. 126:1372-1377); and human fibrosarcoma (HT-1080 cells, ATCC No. CCL-121,) and human mesothelioma (MSTO-211H cells). Exemplary rat tumor xenograft models in mice include, but are not limited to, glioma tumor (C6 cells; ATCC No. CCL-107). Exemplary mouse tumor homograft models include, but are not limited to, mouse melanoma (B16-F10 cells; ATCC No. CRL-6475). Exemplary cat tumor xenograft models in mice include, but are not limited to, feline fibrosarcoma (FC77.T cells; ATCC No. CRL-6105). Exemplary dog tumor xenograft models in mice include, but are not limited to, canine osteosarcoma (D17 cells; ATCC No. CCL-183). Non-limiting examples of human xenograft models and syngeneic tumor models are set forth in the Tables 14 and 15 below.

TABLE 14
Human Tumor Xenograft Models

| | Cell Line | | |
|---|---|---|---|
| Tumor Type | Name | Tumor Type | Cell Line |
| Adenoid cystic | ACC-2 | Kidney carcinoma | Ketr-3 |
| carcinoma | | | |
| Bladder carcinoma | EJ | Leukemia | HL-60 |
| Bladder carcinoma | T24 | Liver carcinoma | Bel-7402 |
| Breast carcinoma | BCaP-37 | Liver carcinoma | HepG-2 |
| Breast carcinoma | MDA-MB-231 | Liver carcinoma | QGY-7701 |
| Breast carcinoma | MX-1 | Liver carcinoma | SMMC7721 |
| Cervical carcinoma | SiHa | Lung carcinoma | A549 |
| Cervical carcinoma | HeLa | Lung carcinoma | NCI-H460 |
| Colon carcinoma | Ls-174-T | Melanoma | A375 |
| Colon carcinoma | CL187 | Melanoma | M14 |
| Colon carcinoma | HCT-116 | Melanoma | MV3 |
| Colon carcinoma | SW116 | Ovary carcinoma | A2780 |
| Gastric carcinoma | MGC-803 | Pancreatic | BXPC-3 |
| | | carcinoma | |
| Gastric carcinoma | SGC-7901 | Prostate carcinoma | PC-3M |
| Gastric carcinoma | BGC-823 | Tongue carcinoma | Tca-8113 |

TABLE 15
Syngeneic Mouse Tumor Model

| Tumor Type | Cell Line Name | Strain of Mice |
|---|---|---|
| Cervical carcinoma | U14 | ICR |
| Liver carcinoma | H22 | ICR |
| Lung carcinoma | Lewis | C57BL6 |
| Melanoma | B16F1, B16F10, B16BL6 | C57BL6 |
| Sarcoma | S180 | ICR |

The route of administration for the modified anti-EGFR antibodies can be any route of administration described herein or known in the art, such as intraperitoneal, intratumoral or intravenous administration. The anti-EGFR antibodies can be administered at varying dosages described herein or known in the art. For example, the modified anti-EGFR antibodies can be administered to tumor-bearing animals at or between, for example, about 0.1 mg/kg, 0.15 mg/kg, 0.2 mg/kg, 0.25 mg/kg, 0.30 mg/kg, 0.35 mg/kg, 0.40 mg/kg, 0.45 mg/kg, 0.5 mg/kg, 0.55 mg·kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 40 mg/kg, 50 mg/kg, 60 mg/kg, 70 mg/kg, 80 mg/kg, 90 mg/kg, 100 mg/kg or more.

In some examples, exemplary dosages include, but are not limited to, about or 0.01 mg/m² to about or 800 mg/m², such as for example, about or 0.01 mg/m², about or 0.1 mg/m², about or 0.5 mg/m², about or 1 mg/m², about or 5 mg/m², about or 10 mg/m², about or 15 mg/m², about or 20 mg/m², about or 25 mg/m², about or 30 mg/m², about or 35 mg/m², about or 40 mg/m², about or 45 mg/m², about or 50 mg/m², about or 100 mg/m², about or 150 mg/m², about or 200 mg/m², about or 250 mg/m², about or 300 mg/m², about or 400 mg/m², about or 500 mg/m², about or 600 mg/m² and about or 700 mg/m². It is understood that one of skill in the art can recognize and convert dosages between units of mg/kg and mg/m² (see, e.g., Michael J. Derelanko, TOXICOLOGIST'S POCKET HANDBOOK, CRC Press, p. 16 (2000)).

Tumor size and volume can be monitored based on techniques known to one of skill in the art. For example, tumor size and volume can be monitored by radiography, ultrasound imaging, necropsy, by use of calipers, by microCT or by ¹⁸F-FDG-PET. Tumor size also can be assessed visually. In particular examples, tumor size (diameter) is measured directly using calipers. In other examples, tumor volume can be measured using an average of measurements of tumor diameter (D) obtained by caliper or ultrasound assessments.

The volume can be determined from the formula V=D³×π/6 (for diameter measured using calipers); the formula V=[length×(width)²]/2 where length is the longest diameter and width is the shortest diameter perpendicular to length; or V=D²×d×π/6 (for diameter measured using ultrasound where d is the depth or thickness). For example, caliper measurements can be made of the tumor length (l) and width (w) and tumor volume calculated as length×width×0.52.

In another example, microCT scans can be used to measure tumor volume (see e.g., Huang et al. (2009) PNAS, 106:3426-3430). In such an example, mice can be injected with Optiray Pharmacy ioversol injection 74% contrast medium (e.g., 741 mg of ioversol/mL), mice anesthetized, and CT scanning done using a MicroCat 1A scanner or other similar scanner (e.g., IMTek) (40 kV, 600 μA, 196 rotation steps, total angle or rotation=196). The images can be reconstructed using software (e.g., RVA3 software program; ImTek). Tumor volumes can be determined by using available software (e.g., Amira 3.1 software; Mercury Computer Systems). In some examples, the tumor is injected subcutaneously at day 0, and the volume of the primary tumor can be measured at designated time points.

Once the implanted tumors reach a predetermined size or volume, the modified anti-EGFR antibody can be administered. Progressing tumors can be visualized and tumor size and tumor volume can be measured using any technique known to one of skill in the art. For example, tumor volume or tumor size can be measured using any of the techniques described herein. Tumor volume and size can be assessed or measured at periodic intervals over a period of time following administration of the modified anti-EGFR antibodies provided herein, such as, for example, every hour, every 6 hours, every 12 hours, every 24 hours, every 36 hours, every 2 days, every 3 days, every 4 days, every 5 days, every 6 days, every 7-days, every week, every 3 weeks, every month or more post-infection. A graph of the median change in tumor volume over time can be made. This is exemplified in Example 10. The total area under the curve (AUC) can be calculated. A therapeutic index also can be calculated using the formula AUC_{untreated animals}−AUC_{treated animals}/AUC_{untreated}×100.

Generally, tumor-bearing animals generated in the same manner, at the same time and with the same type of tumor cells are used as controls. Such control tumor-bearing animals include those that remain untreated (not administered modified anti-EGFR antibody). Additional controls animals include those administered an anti-EGFR antibody known in the art. An example of such anti-EGFR antibodies is Cetuximab. In examples where tumor-bearing animals are administered a known anti-EGFR antibody as a control, the amount of control antibody administered can be the same as the amount of the modified anti-EGFR antibody.

Assessment of the activity of a modified anti-EGFR antibody can include identifying antibodies that mediate a decrease in tumor size (e.g., diameter), volume or weight compared to control treated or untreated tumor-bearing animals. It is understood that a decrease in tumor size, volume or weight compared to control treated or untreated tumor-bearing animals means that the anti-EGFR antibody itself is mediating tumor regression or shrinkage or that the anti-EGFR antibody is mediating delayed tumor progression compared to control treated or untreated tumor-bearing animals. Tumor shrinkage or delay in tumor progression are parameters indicative of anti-tumorigenicity.

For example, an anti-EGFR antibody can be identified as mediating a decrease in tumor size or volume based on visual assessment of tumor size in the animal compared to control treated or untreated tumor-bearing animals. In other examples, an anti-EGFR antibody is identified as mediating a decrease in tumor size or volume if the tumor size is decreased in diameter as assessed by any measurement known in the art (e.g., use of calipers) compared to an untreated tumor-bearing animal or compared to a tumor-bearing animal treated with a reference anti-EGFR antibody. It is understood that comparison of tumor size or volume can be made at any predetermined time post-infection, and can be empirically determined by one of skill in the art. In some examples, a comparison can be made at the day in which the untreated control is sacrificed. In other examples, analysis of the total AUC can be made, and AUC values compared as an indicator of the size and volume of the tumor over the time period.

Effects of a modified anti-EGFR antibody on tumor size or volume can be presented as a ratio of tumor size or volume at a designated time post-administration of the control treated animal compared to the anti-EGFR antibody-treated animal (tumor size or volume of control-treated animals/tumor size or volume of modified anti-EGFR antibody-treated animals). Assessment can include identifying an anti-EGFR antibody that results in animals exhibiting a ratio of tumor shrinkage that is greater than 1.0, for example, that is greater than 1.1, 1.2, 1.3, 1.4, 1.5, 1.6, 1.7, 1.8, 1.9, 2, 3, 4, 5, 6, 7, 8, 9, 10, 20, 30, 40, 50 or more. In particular examples, the results are presented as a ratio of the total AUC area during the course of treatment (AUC of tumor size or volume of control-treated animals/AUC tumor size or volume of modified anti-EGFR antibody-treated animals). An anti-EGFR antibody can be selected that results in a ratio of tumor shrinkage in a subject as measured by AUC that is greater than 1.1, 1.2, 1.3, 1.4, 1.5, 1.6, 1.7, 1.8, 1.9, 2, 3, 4, 5, 6, 7, 8, 9, 10, 20, 30, 40, 50 or more. It is understood that a ratio of 1.2 or 5 means that the modified anti-EGFR antibody effects a decreased tumor size or volume and results in 120% or 500% anti-tumorigenicity activity compared to the reference or control.

In particular examples, the therapeutic index is determined as a measure of effects of an anti-EGFR antibody, such as a modified anti-EGFR antibody, on tumor size or volume. An anti-EGFR antibody can have a therapeutic index that is at least or about at least or 120%, 130%, 140%, 150%, 160%, 170%, 180%, 190%, 200%, 250%, 300%, 400%, 500%, 600%, 700%, 800% or more compared to the therapeutic index of a control anti-EGFR antibody.

In additional examples, tumors can be harvested from the animals and weighed. Administration of anti-EGFR antibodies can result in a decrease in tumor weight compared to tumor harvested from control tumor-bearing animals. The weight also can be compared to tumors harvested from control treated animals at the same time post-administration. The change in weight can be presented as a ratio of the tumor weight (tumor weight control treated animals/tumor weights of anti-EGFR-treated animals). An anti-EGFR antibody can result in subjects exhibiting a ratio of tumor weight that is greater than 1.0, for example, that is greater than 1.1, 1.2, 1.3, 1.4, 1.5, 1.6, 1.7, 1.8, 1.9, 2, 3, 4, 5, 6, 7, 8, 9, 10, 20, 30, 40, 50 or more. It is understood that a ratio of tumor weight that is 1.2 or 5 means that the anti-EGFR antibody effects a decreased tumor weight and results in 120% or 500% anti-tumorigenicity activity compared to the reference or control.

In particular examples, the effect of the anti-EGFR antibody on other organs or tissues in the animal can be assessed. For example, other organs can be harvested from the animals, weighed and/or examined.

Animal studies also can be performed to assess adverse side effects, such as side effects that cannot be evaluated in a standard pharmacology profile or occur only after repeated administration of the modified anti-EGFR antibody. The assessed side effects of a modified anti-EGFR antibody can include any side effect of anti-EGFR antibodies described herein or known in the art, including skin toxicities and hypomagnesemia. For example, known side effects of Cetuximab include any described herein and/or known to one of skill in the art, including symptomatic hypomagnesemia, paronychia, fever, dermatologic toxicity, papulopustular rash of the face and upper trunk, hair growth abnormalities, loss of scalp hair, increased growth of facial hair and eyelashes, dry and itchy skin, and periungual inflammation with tenderness (Eng (2009) Nat. Rev. 6:207-218; Schrag et al. J. Natl. Cancer Inst. 97(16):1221-1224; Lacouture, and Melosky (2007) Skin Therapy Lett. 12:1-5). Other parameters that can be measured to assess side effects include standard measurement of food consumption, bodyweight, antibody formation, clinical chemistry, and macro- and microscopic examination of standard organs/tissues (e.g., cardiotoxicity). Additional parameters of measurement include injection site trauma and the measurement of any neutralizing antibodies.

For example, as described elsewhere herein, hypomagnesemia can be diagnosed and/or assessed by measurement of serum magnesium levels. Papulopustular rash and acneiform rash can be characterized in animal models, such as mouse models and cynomolgus monkey models, by observing eruptions consisting of papules (a small, raised pimple) and pustules (a small pus filled blister). Dry skin, can be characterized by flaky and dull skin, fine pores, and papery thin skin texture. Skin hyperpigmentation can be characterized by darkening of the skin due to excessive melanin deposition. Pruritus can be evaluated by observing animal scratching. Paronychia can be evaluated by examination.

In some examples, the presence of skin toxicities can be evaluated in mouse models in which human skin is grafted onto mice (see, e.g., Nanney et al. (1996) J. Invest. Dermatol. 106(6):1169-1174). It addition, dermatologic side effects can be assessed in other animal models. For example, in cynomolgus monkeys, inflammation at the injection site and desquamation of the external integument after cetuximab administration can be assessed. Similar effects can be observed in the epithelial mucosa of the nasal passage, esophagus, and tongue, and degenerative changes in the renal tubular epithelium. Other epithelial toxicities that can be assessed include conjunctivitis, reddened and swollen eyes, and signs of intestinal disturbance (see, e.g., Lutterbuese et al. (2010) Proc. Natl. Acad. Sci. 107(28):12605-12610; European Medicines Agency (2009) Summary of product characteristics (Erbitux)).

Side effects can be assessed in healthy animal models or in animal models of a disease or condition, such as the animal models described herein. In some examples, such assays can be performed in two species (e.g., a rodent and a non-rodent) to ensure that any unexpected adverse effects are not overlooked. In general, these models can measure a variety of toxicities including genotoxicity, chronic toxicity, immunogenicity, reproductive/developmental toxicity, carcinogenicity.

4. Pharmacokinetics and Pharmacodynamics Assays

Pharmacokinetics (PK) and pharmacodynamics (PD) assays of the modified anti-EGFR antibodies provided herein can be performed using methods described herein or known in the art (see, e.g., Klutchko, et al., (1998) J. Med. Chem. 41:3276-3292). Examples of parameters of measurement generally include the maximum (peak) plasma concentration (Cₘₐₓ), the peak time (i.e., when maximum plasma concentration occurs; Tₘₐₓ), the minimum plasma concentration (i.e., the minimum plasma concentration between doses; Cₘᵢₙ), the elimination half-life (T_{1/2}) and area under the curve (i.e., the area under the curve generated by plotting time versus plasma concentration; AUC), following administration. The absolute bioavailability of administered modified anti-EGFR antibody can be determined by comparing the area under the curve following subcutaneous delivery (AUC_{sc}) with the AUC following intravenous delivery (AUCᵢᵥ). Absolute bioavailability (F), can be calculated using the formula: F=([AUC]_{sc}×dose_{sc})/([AUC]ᵢᵥ×doseᵢᵥ). The concentration of anti-EGFR antibody in the plasma following administration can be measured using any method known in the art suitable for assessing concentrations of antibody in samples of blood. Exemplary methods include, but are not limited to, ELISA and nephelometry. Additional measured parameters can include compartmental analysis of concentration-time data obtained following intravenous administration and bioavailability. Biodistribution, dosimetry (for radiolabeled antibodies or Fc fusions), and PK studies can also be done in animal models, including animal models described herein or known in the art, including rodent models. Such studies can evaluate tolerance at some or all doses administered, toxicity to local tissues, preferential localization to rodent xenograft animal models and depletion of target cells (e.g., CD20 positive cells). Pharmacodynamic studies can include, but are not limited to, targeting specific tumor cells or blocking signaling mechanisms, measuring depletion of EGFR expressing cells or signals.

PK and PD assays can be performed in any animal model described herein or known in the art, including healthy animal models, diseased animal models and humans. Screening the modified anti-EGFR antibodies for PD and/or PK properties can be useful for defining the optimal balance of PD, PK, and therapeutic efficacy conferred by the modified anti-EGFR antibodies. For example, it is known in the art that the array of Fc receptors is differentially expressed on various immune cell types, as well as in different tissues. Differential tissue distribution of Fc receptors can affect the pharmacodynamic (PD) and pharmacokinetic (PK) properties of the modified anti-EGFR antibodies provided herein.

A range of doses and different dosing frequency of dosing can be administered in the pharmacokinetic studies to assess the effect of increasing or decreasing concentrations of the modified anti-EGFR antibody in the dose. Pharmacokinetic properties, such as bioavailability, of the administered modified anti-EGFR antibody, can be assessed with or without co-administration of a therapeutic agent or regimen described herein. For example, dogs, such as beagles, can be administered a modified anti-EGFR antibody alone or with one or more therapeutic agents or regimens described herein. The modified anti-EGFR antibody can be administered before, during or after administration of a therapeutic agent or regimen. Blood samples can then be taken at various time points and the amount of modified anti-EGFR antibody in the plasma determined, such as by nephelometry. The AUC can then be measured and the bioavailability of administered modified anti-EGFR antibody with or without co-administration of the additional therapeutic agent(s) or regimen(s) can be determined Such studies can be performed to assess the effect of co-administration on pharmacokinetic properties, such as bioavailability, of administered anti-EGFR antibody.

Single or repeated administration(s) of the modified anti-EGFR antibodies can occur over a dose range of about 6000-fold (about 0.05-300 mg/kg) to evaluate the half-life using plasma concentration and clearance as well as volume of distribution at a steady state and level of systemic absorbance can be measured.

### F. PHARMACEUTICAL COMPOSITIONS, FORMULATIONS, KITS, ARTICLES OF MANUFACTURE AND COMBINATIONS

1. Pharmaceutical Compositions and Formulations

Pharmaceutical compositions containing any of the modified anti-EGFR antibodies, or antigen-binding fragments thereof, provided herein are provided for administration. Pharmaceutically acceptable compositions are prepared in view of approvals for a regulatory agency or other agency prepared in accordance with generally recognized pharmacopeia for use in animals and in humans. Typically, the compounds are formulated into pharmaceutical compositions using techniques and procedures well-known in the art (see e.g., Ansel Introduction to Pharmaceutical Dosage Forms, Fourth Edition, 1985, 126).

The pharmaceutical composition can be used for therapeutic, prophylactic, and/or diagnostic applications. The anti-EGFR antibodies provided herein can be formulated with a pharmaceutical acceptable carrier or diluent. Generally, such pharmaceutical compositions utilize components which will not significantly impair the biological properties of the antibody, such as the binding of to its specific epitope (e.g., binding to EGFR). Each component is pharmaceutically and physiologically acceptable in the sense of being compatible with the other ingredients and not injurious to the patient. The formulations can conveniently be presented in unit dosage form and can be prepared by methods well-known in the art of pharmacy, including but not limited to, tablets, pills, powders, liquid solutions or suspensions (e.g., including injectable, ingestible and topical formulations (e.g., eye drops, gels, pastes, creams, or ointments), aerosols (e.g., nasal sprays), liposomes, suppositories, pessaries, injectable and infusible solution and sustained release forms. See, e.g., Gilman, et al. (eds. 1990) Goodman and Gilman's: The Pharmacological Bases of Therapeutics, 8th Ed., Pergamon Press; and Remington's Pharmaceutical Sciences, 17th ed. (1990), Mack Publishing Co., Easton, Pa.; Avis, et al. (eds. 1993) Pharmaceutical Dosage Forms: Parenteral Medications Dekker, NY; Lieberman, et al. (eds. 1990) Pharmaceutical Dosage Forms: Tablets Dekker, NY; and Lieberman, et al. (eds. 1990) Pharmaceutical Dosage Forms: Disperse Systems Dekker, NY. When administered systematically, the therapeutic composition is sterile, pyrogen-free, generally free of particulate matter, and in a parenterally acceptable solution having due regard for pH, isotonicity, and stability. These conditions are known to those skilled in the art. Methods for preparing parenterally administrable compositions are well-known or will be apparent to those skilled in the art and are described in more detail in, e.g., “Remington: The Science and Practice of Pharmacy (Formerly Remington's Pharmaceutical Sciences)”, 19th ed., Mack Publishing Company, Easton, Pa. (1995).

Pharmaceutical compositions provided herein can be in various forms, e.g., in solid, semi-solid, liquid, powder, aqueous, or lyophilized form. Examples of suitable pharmaceutical carriers are known in the art and include but are not limited to water, buffering agents, saline solutions, phosphate buffered saline solutions, various types of wetting agents, sterile solutions, alcohols, gum arabic, vegetable oils, benzyl alcohols, gelatin, glycerin, carbohydrates such as lactose, sucrose, amylose or starch, magnesium stearate, talc, silicic acid, viscous paraffin, perfume oil, fatty acid monoglycerides and diglycerides, pentaerythritol fatty acid esters, hydroxy methylcellulose, powders, among others. Pharmaceutical compositions provided herein can contain other additives including, for example, antioxidants, preservatives, antimicrobial agents, analgesic agents, binders, disintegrants, coloring, diluents, excipients, extenders, glidants, solubilizers, stabilizers, tonicity agents, vehicles, viscosity agents, flavoring agents, emulsions, such as oil/water emulsions, emulsifying and suspending agents, such as acacia, agar, alginic acid, sodium alginate, bentonite, carbomer, carrageenan, carboxymethylcellulose, cellulose, cholesterol, gelatin, hydroxyethyl cellulose, hydroxypropyl cellulose, hydroxypropyl methylcellulose, methylcellulose, octoxynol-9, oleyl alcohol, povidone, propylene glycol monostearate, sodium lauryl sulfate, sorbitan esters, stearyl alcohol, tragacanth, xanthan gum, and derivatives thereof, solvents, and miscellaneous ingredients such as crystalline cellulose, microcrystalline cellulose, citric acid, dextrin, dextrose, liquid glucose, lactic acid, lactose, magnesium chloride, potassium metaphosphate, starch, among others (see, generally, Alfonso R. Gennaro (2000) Remington: The Science and Practice of Pharmacy, 20th Edition. Baltimore, Md.: Lippincott Williams & Wilkins). Such carriers and/or additives can be formulated by conventional methods and can be administered to the subject at a suitable dose. Stabilizing agents such as lipids, nuclease inhibitors, polymers, and chelating agents can preserve the compositions from degradation within the body.

The route of antibody administration is in accord with known methods, e.g., injection or infusion by intravenous, intraperitoneal, intracerebral, intramuscular, subcutaneous, intraocular, intraarterial, intrathecal, inhalation or intralesional routes, topical or by sustained release systems as noted below. The antibody is typically administered continuously by infusion or by bolus injection. One can administer the antibodies in a local or systemic manner.

The anti-EGFR antibodies, such as modified antibodies, provided herein can be prepared in a mixture with a pharmaceutically acceptable carrier. Techniques for formulation and administration of the compounds are known to one of skill in the art (see e.g., “Remington's Pharmaceutical Sciences,” Mack Publishing Co., Easton, Pa.). This therapeutic composition can be administered intravenously or through the nose or lung, preferably as a liquid or powder aerosol (lyophilized). The composition also can be administered parenterally or subcutaneously as desired. When administered systematically, the therapeutic composition should be sterile, pyrogen-free and in a parenterally acceptable solution having due regard for pH, isotonicity, and stability. These conditions are known to those skilled in the art. Pharmaceutical compositions suitable for use include compositions wherein one or more anti-EGFR antibodies are contained in an amount effective to achieve their intended purpose. Determination of a therapeutically effective amount is well within the capability of those skilled in the art. Therapeutically effective dosages can be determined by using in vitro and in vivo methods as described herein. Accordingly, an anti-EGFR antibody provided herein, when in a pharmaceutical preparation, can be present in unit dose forms for administration.

Therapeutic formulations can be administered in many conventional dosage formulations. Briefly, dosage formulations of the antibodies provided herein are prepared for storage or administration by mixing the compound having the desired degree of purity with physiologically acceptable carriers, excipients, or stabilizers. Such materials are non-toxic to the recipients at the dosages and concentrations employed, and can include buffers such as TRIS HCl, phosphate, citrate, acetate and other organic acid salts; antioxidants such as ascorbic acid; low molecular weight (less than about ten residues) peptides such as polyarginine, proteins, such as serum albumin, gelatin, or immunoglobulins; hydrophilic polymers such as polyvinylpyrrolidinone; amino acids such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium and/or nonionic surfactants such as TWEEN, PLURONICS or polyethylene glycol.

In particular examples herein, provided herein are pharmaceutical compositions that contain a stabilizing agent. The stabilizing agent can be an amino acid, amino acid derivative, amine, sugar, polyols, salt or surfactant. In some examples, the stable co-formulations contain a single stabilizing agent. In other examples, the stable co-formulations contain 2, 3, 4, 5 or 6 different stabilizing agents.

For example, the stabilizing agent can be a sugar or polyol, such as a glycerol, sorbitol, mannitol, inositol, sucrose or trehalose. In particular examples, the stabilizing agent is sucrose. In other examples, the stabilizing agent is trehalose. The concentration of the sugar or polyol is from or from about 100 mM to 500 mM, 100 mM to 400 mM, 100 mM to 300 mM, 100 mM to 200 mM, 200 mM to 500 mM, 200 mM to 400 mM, 200 mM to 300 mM, 250 mM to 500 mM, 250 mM to 400 mM, 250 mM to 300 mM, 300 mM to 500 mM, 300 mM to 400 mM, or 400 mM to 500 mM, each inclusive.

In examples, the stabilizing agent can be a surfactant that is a polypropylene glycol, polyethylene glycol, glycerin, sorbitol, poloxamer and polysorbate. For example, the surfactant can be a polypropylene glycol, polyethylene glycol, glycerin, sorbitol, poloxamer and polysorbate, such as a poloxamer 188, polysorbate 20 and polysorbate 80. In particular examples, the stabilizing agent is polysorbate 80. The concentration of surfactant, as a % of mass concentration (w/v) in the formulation, is between or about between 0.005% to 1.0%, 0.01% to 0.5%, 0.01% to 0.1%, 0.01% to 0.05%, or 0.01% to 0.02%, each inclusive.

When used for in vivo administration, the modified anti-EGFR antibody formulation should be sterile and can be formulated according to conventional pharmaceutical practice. This is readily accomplished by filtration through sterile filtration membranes, prior to or following lyophilization and reconstitution. The antibody ordinarily will be stored in lyophilized form or in solution. Other vehicles such as naturally occurring vegetable oil like sesame, peanut, or cottonseed oil or a synthetic fatty vehicle like ethyl oleate or the like may be desired. Buffers, preservatives, antioxidants and the like can be incorporated according to accepted pharmaceutical practice.

The anti-EGFR antibodies, such as modified anti-EGFR antibodies, can be provided at a concentration in the composition of from or from about 0.1 to 10 mg/mL, such as, for example a concentration that is at least or at least about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10 mg/mL or more. The volume of the solution can be at or about 1 to 100 mL, such as, for example, at least or about at least or 0.5, 1, 2, 3, 4, 5, 10, 15, 20, 25, 30, 35, 40, 45, 50, 55, 60, 65, 70, 75, 80, 85, 90, 95, 100 mL or more. In some examples, the anti-EGFR antibodies are supplied in phosphate buffered saline. For example, the anti-EGFR antibodies can be supplied as a 50-mL, single-use vial containing 100 mg of anti-EGFR antibody at a concentration of 2 mg/mL in phosphate buffered saline.

An anti-EGFR antibody provided herein can be lyophilized for storage and reconstituted in a suitable carrier prior to use. This technique has been shown to be effective with conventional immunoglobulins and protein preparations and art-known lyophilization and reconstitution techniques can be employed.

An anti-EGFR antibody provided herein can be provided as a controlled release or sustained release composition. Polymeric materials are known in the art for the formulation of pills and capsules which can achieve controlled or sustained release of the antibodies provided herein (see, e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Langer and Peppas (1983) J. Macromol. Sci. 23:61; see also Levy et al. (1985) Science 228:190; During et al. (1989) Ann. Neurol. 25:351; Howard et al. (1989) J. Neurosurg. 71:105; U.S. Pat. Nos. 5,679,377, 5,916,597, 5,912,015, 5,989,463, 5,128,326; and PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. Generally, the polymer used in a sustained release formulation is inert, free of leachable impurities, stable on storage, sterile, and biodegradable. Any technique known in the art for the production of sustained release formulation can be used to produce a sustained release formulation containing one more anti-EGFR antibodies provided herein.

In some examples, the pharmaceutical composition contains an anti-EGFR antibody provided herein and one or more additional antibodies. In some examples, the one or more additional antibodies includes, but is not limited to, anti-EGFR antibodies described herein or known in the art, such as, for example, ABX-EGF or cetuximab.

2. Articles of Manufacture/Kits

Pharmaceutical compositions of modified anti-EGFR antibodies or nucleic acids encoding modified anti-EGFR antibodies, or a derivative or a biologically active portion thereof, can be packaged as articles of manufacture containing packaging material, a pharmaceutical composition which is effective for treating a disease or conditions that can be treated by administration of an anti-EGFR antibody, such as the diseases and conditions described herein or known in the art, and a label that indicates that the antibody or nucleic acid molecule is to be used for treating the infection, disease or disorder. The pharmaceutical compositions can be packaged in unit dosage forms containing an amount of the pharmaceutical composition for a single dose or multiple doses. The packaged compositions can contain a lyophilized powder of the pharmaceutical compositions containing the modified anti-EGFR antibodies provided, which can be reconstituted (e.g., with water or saline) prior to administration.

The articles of manufacture provided herein contain packaging materials. Packaging materials for use in packaging pharmaceutical products are well-known to those of skill in the art (see, e.g., U.S. Pat. Nos. 5,323,907, 5,052,558 and 5,033,252). Examples of pharmaceutical packaging materials include, but are not limited to, blister packs, bottles, tubes, inhalers (e.g., pressurized metered dose inhalers (MDI), dry powder inhalers (DPI), nebulizers (e.g., jet or ultrasonic nebulizers) and other single breath liquid systems), pumps, bags, vials, containers, syringes, bottles, and any packaging material suitable for a selected formulation and intended mode of administration and treatment.

The modified anti-EGFR antibodies, nucleic acid molecules encoding the antibodies, pharmaceutical compositions, or combinations provided herein also can be provided as kits. Kits can optionally include one or more components such as instructions for use, devices and additional reagents (e.g., sterilized water or saline solutions for dilution of the compositions and/or reconstitution of lyophilized protein), and components, such as tubes, containers and syringes for practice of the methods. Exemplary kits can include the anti-EGFR antibodies provided herein, and can optionally include instructions for use, a device for administering the anti-EGFR antibodies to a subject, a device for detecting the anti-EGFR antibodies in a subject, a device for detecting the anti-EGFR antibodies in samples obtained from a subject, and a device for administering an additional therapeutic agent to a subject.

The kit can, optionally, include instructions. Instructions typically include a tangible expression describing the modified anti-EGFR antibodies and, optionally, other components included in the kit, and methods for administration, including methods for determining the proper state of the subject, the proper dosage amount, dosing regimens, and the proper administration method for administering the anti-EGFR antibodies. Instructions also can include guidance for monitoring the subject over the duration of the treatment time.

Kits also can include a pharmaceutical composition described herein and an item for diagnosis. For example, such kits can include an item for measuring the concentration, amount or activity of the selected anti-EGFR antibody in a subject.

In some examples, the anti-EGFR antibody is provided in a diagnostic kit for the detection of EGFR in an isolated biological sample (e.g., tumor cells, such as circulating tumor cells obtained from a subject or tumor cells excised from a subject). In some examples, the diagnostic kit contains a panel of one or more anti-EGFR antibodies and/or one or more control antibodies (i.e., non-EGFR binding antibodies or EGFR antibodies known in the art, such as cetuximab), where one or more antibodies in the panel is a modified anti-EGFR antibody provided herein.

Kits provided herein also can include a device for administering the anti-EGFR antibodies to a subject. Any of a variety of devices known in the art for administering medications to a subject can be included in the kits provided herein. Exemplary devices include, but are not limited to, a hypodermic needle, an intravenous needle, and a catheter. Typically the device for administering the modified anti-EGFR antibodies of the kit will be compatible with the desired method of administration of the modified anti-EGFR antibodies.

3. Combinations

Provided are combinations of the modified anti-EGFR antibodies provided herein and a second agent, such as a second anti-EGFR antibody or other therapeutic or diagnostic agent. A combination can include any anti-EGFR antibody or reagent for effecting therapy thereof in accord with the methods provided herein. For example, a combination can include any anti-EGFR antibody and a chemotherapeutic agent. Combinations also can include an anti-EGFR antibody provided herein with one or more additional therapeutic antibodies. For example, the additional therapeutic agent is an anti-cancer agent, such as a chemotherapeutic agent, for example, as described in Section G. Combinations of the modified anti-EGFR antibodies thereof provided also can contain pharmaceutical compositions containing the anti-EGFR antibodies or host cells containing nucleic acids encoding the anti-EGFR antibodies as described herein. The combinations provided herein can be formulated as a single composition or in separate compositions.

### G. THERAPEUTIC USES

The anti-EGFR antibodies, or fragments thereof, provided herein can be used for any purpose known to the skilled artisan for use of an anti-EGFR antibody. For example, the anti-EGFR antibodies described herein can be used for one or more of therapeutic, diagnostic, industrial and/or research purpose(s). In particular, the methods provided herein include methods for the therapeutic uses of the modified anti-EGFR antibodies provided herein. In some examples, the anti-EGFR antibodies described herein can be used to kill target cells that include EGFR, such as, for example cancer cells. In some examples, the anti-EGFR antibodies can block, antagonize, or agonize EGFR. By virtue of such activity, the anti-EGFR antibodies provided herein, or fragments thereof, can be administered to a patient or subject for treatment of any condition responsive to treatment with an anti-EGFR antibody, including, but not limited to, a tumor, cancer or metastasis. The therapeutic uses include administration of a therapeutically effective amount of an anti-EGFR antibody, alone or in combination with other treatments or agents.

The anti-EGFR antibodies, such as modified anti-EGFR antibodies and fragments thereof, provided herein, can be used as therapeutics for the treatment of any disease or condition in which existing anti-EGFR antibodies, such as cetuximab, are used. The anti-EGFR antibodies, when administered, result in subjects exhibiting reduced or lessened side effects compared to side effects that can be observed after administration of other anti-EGFR antibodies. Treatment of diseases and conditions with anti-EGFR antibodies, such as modified anti-EGFR antibodies, can be effected by any suitable route of administration using suitable formulations as described herein including, but not limited to, infusion, subcutaneous injection, intramuscular, intradermal, oral, and topical and transdermal administration.

As discussed elsewhere herein, existing anti-EGFR antibodies, such as Cetuximab, when administered, can result in subjects exhibiting local and systemic side effects, and, in particular, dermal side effects. These side effects limit the therapeutic use. In many cases, these side effects are associated with binding to EGFR at a neutral physiologic pH environment, such as in the skin dermis. The modified anti-EGFR antibodies provided herein, which are more active under conditions that include one or both of low pH ranging from about 5.6 to about 6.8, and in particular 6.0 to 6.5, inclusive, and/or lactate concentration of 15 mM to 20 mM (e.g., 16.6 mM or 16.7 mM) compared to under conditions that contain one or both of neutral pH (e.g., pH about or 7.0 to 7.4, inclusive) and/or normal lactate concentrations of 0.5 mM to 5 mM (e.g., 1 mM), can be administered for the treatment of any disease or condition described herein. By virtue of the activity, the modified anti-EGFR antibodies provided herein can have greater activity in a tumor environment (which can have a low pH and/or increased lactic acid concentrations) than in a neutral physiologic environment that is associated with one or more side effects of an anti-EGFR antibody, such as the skin basal layer. This can be advantageous by targeting therapy only to diseased tissues, such as tumor tissues, in order to reduce or prevent side effects, including local and systemic side effects.

Hence, the modified anti-EGFR antibodies provided herein that are associated with reduced side effects, such as the modified anti-EGFR antibodies provided herein, can be used at higher dosing regimens, and can have improved efficacy and safety. Side effects that can be reduced compared to those observed by existing anti-EGFR antibody therapeutics, such as Cetuximab, include any undesirable nontherapeutic effect described herein or known in the art, such as nausea, emesis, chest tightness, headache, and related cardiovascular effects such as blood pressure instability and arterial constriction, dermal toxicity, bone marrow suppression, cardiotoxicity, hair loss, renal dysfunctions, stomatitis, anemia, seizures, immune reactions such as acute anaphylaxis, serum sickness, generation of antibodies, infections, cancer, autoimmune disease and cardiotoxicity. In some examples, compared to side effects caused by administration of existing anti-EGFR antibody therapeutics, such as Cetuximab, administration of a modified anti-EGFR antibody provided herein decreases the severity of one or more side effects by at least or about 99%, at least or about 95%, at least or about 90%, at least or about 85%, at least or about 80%, at least or about 75%, at least or about 70%, at least or about 65%, at least or about 60%, at least or about 55%, at least or about 50%, at least or about 45%, at least or about 40%, at least or about 35%, at least or about 30%, at least or about 25%, at least or about 20%, at least or about 15%, or at least or about 10% relative to the severity of the one or more side effects of an unmodified EGFR antibody.

It is understood that while the anti-EGFR antibodies, such as modified anti-EGFR antibodies, and antibody fragments, provided herein, when administered, can result in subjects exhibiting lessened or reduced side effects compared to other anti-EGFR antibodies, such as Cetuximab, that some side effects can occur upon administration. It is understood that number and degree of tolerable side effects depends upon the condition for which the compounds are administered. For example, certain toxic and undesirable side effects are tolerated when treating life-threatening illnesses that would not be tolerated when treating disorders of lesser consequence. Amounts effective for therapeutic use can depend on the severity of the disease and the weight and general state of the subject as well as the route of administration. Local administration of the therapeutic agent will typically require a smaller dosage than any mode of systemic administration, although the local concentration of the therapeutic agent can, in some cases, be higher following local administration than can be achieved with safety upon systemic administration.

This section provides exemplary uses of, and administration methods for, the modified anti-EGFR antibodies, provided herein. These described uses are exemplary and do not limit the applications of the antibodies described herein. It is within the skill of a treating physician to identify diseases or conditions which are treatable using an anti-EGFR antibody.

1. Exemplary Diseases and Conditions

The modified anti-EGFR antibodies described herein can be used for any therapeutic purpose for which anti-EGFR antibodies can be used (see, e.g., Reeves et al. (2011) Otolaryngol Head Neck Surg. 144(5):676-84; Adams et al. (2008) Expert Rev Anticancer Ther. 8(8):1237-45; Belda-Iniesta et al. (2006) Cancer Biol Ther. 5(8):912-4; Liu et al. (2010) Cancer Chemother Pharmacol. 65(5):849-61). In some examples, the anti-EGFR antibodies are administered to a patient to treat a disease or disorder that can be treated with an anti-EGFR antibody. In some examples, treatment of the disease includes administration of a modified anti-EGFR antibody described herein after clinical manifestation of the disease to combat the symptoms of the disease. In some examples, administration of a modified anti-EGFR antibody described herein is administered to eradicate the disease. Examples of diseases or disorders that can be treated with the modified anti-EGFR antibodies described herein include autoimmune and inflammatory diseases, infectious diseases, and cancer.

a. Cancer

EGFR is associated with cancer development and progression in a variety of human malignancies, such as lung cancer, head and neck cancer, colon cancer, breast cancer, ovarian cancer and glioma. EGFR-related molecular factors, such as copy number and gene mutations, have been identified as prognostic and predictive factors for cancer (see, e.g., Bronte et al. (2011) Front Biosci. 3:879-887; Harding and Burtness (2005) Drugs Today 41(2):107-127). For example, high EGFR expression is associated with poor prognosis in patients with head and neck squamous cell carcinoma (HNSCC) (Szabo et al. (2011) Oral Oncol. 47(6):487-496).

The modified anti-EGFR antibodies described herein, can bind to and prevent stimulation of the EGF receptor. Due to the pH selective binding, the binding activity is selective to tumor microenvironments that exhibit one or both of acidic pH and elevated lactate concentrations. For example, an altered pH microenvironment is the most common microenvironment found in disease states such as tumor microenvironments, and it is the most uniform within the disease microenvironment compared to other properties such as hypoxia (see e.g., Fogh Andersen et al. (1995) Clin. Chem., 41:1522-1525; Bhujwalla et al. (2002) NMR Biomed., 15:114-119; Helmlinger et al. (1997) Nature Med., 3:177; Gerweck and Seetharaman (1996), Cancer Res. 56(6):1194-1198). For example, in many tumors the ‘Warburg effect’ creates a microenvironment with a pH ranging from about 5.6 to about 6.8, such as less than or about or pH 5.6, 5.7, 5.8, 5.9, 6.0, 6.1, 6.2, 6.3, 6.4, 6.5, 6.6, 6.7, or 6.8. Thus, anti-EGFR antibodies that are more active at acidic pH than at neutral pH, such as the modified anti-EGFR antibodies described herein, can be used to treat EGFR expressing tumors, while minimizing activity in non-target disease cells or tissues.

In addition, in many tumors, the ‘Warburg effect’ creates a microenvironment with lactate concentrations between 10 to 20 mM. Elevated lactate levels have been found associated with a variety of tumors including, but not limited to, head and neck, metastatic colorectal cancer, cervical cancer and squamous cell carcinoma (see, e.g., Walenta et al., (1997) American Journal of Pathology 150(2): 409-415; Schwickert et al., (1995) Cancer Research 55: 4757-4759; Walenta et al., (2000) Cancer Research 60: 916-921; Guo et al., (2004) J Nucl Med 45: 1334-1339; Mathupala et al., (2007) J Bioenerg Biomembr 39: 73-77; Holroyde et al., (1979) Cancer Research 39: 4900-4904; Schurr and Payne. (2007) Neuroscience 147: 613-619; and Quennet et al., (2006) Radiotherapy and Oncology 81: 130-135). Thus, anti-EGFR antibodies that are more active at increased lactate concentrations than at normal physiologic lactate concentrations, such as the modified anti-EGFR antibodies described herein, can be used to treat EGFR expressing tumors, while minimizing activity at non-target disease cells or tissues.

The binding of modified anti-EGFR antibodies to EGFR can inhibit the functional activity of the receptor. For example, binding of a modified anti-EGFR antibody to the receptor can inhibit the binding of epidermal growth factor (EGF) and/or result in internalization of the antibody-receptor complex (Harding and Burtness, Drugs Today (Barc)). Thus, anti-EGFR antibodies, such as the modified anti-EGFR antibodies provided herein, can, for example, prevent receptor phosphorylation and activation of the receptor-associated kinase activity, ultimately shutting off receptor-mediated cell signaling.

Modified anti-EGFR antibodies, and fragments thereof, described herein, can be used to treat tumors, including solid tumors, that express EGFR. EGFR expressing tumors can be sensitive to EGF present in their local microenvironment, and can further be stimulated by tumor produced EGF or Transforming Growth Factor-alpha (TGF-α). The diseases and conditions that can be treated or prevented by administering the provided modified anti-EGFR antibodies include, for example, those in which tumor growth is stimulated through an EGFR paracrine and/or autocrine loop. The treatments described herein can therefore be useful for treating a tumor that is not vascularized, or is not yet substantially vascularized.

In addition, the modified anti-EGFR antibodies described herein can inhibit tumor-associated angiogenesis. EGFR stimulation of vascular endothelium is associated with vascularization of tumors. Typically, vascular endothelium is stimulated in a paracrine fashion by EGF and/or TGF-α from other sources (e.g., tumor cells). Accordingly, anti-EGFR antibodies, such as the modified anti-EGFR antibodies described herein, can be useful for treating subjects with vascularized tumors or neoplasms.

Tumors that can be treated include primary tumors and metastatic tumors, as well as refractory tumors. Refractory tumors include tumors that fail to respond or are resistant to treatment with chemotherapeutic agents alone, antibodies alone, radiation alone or combinations thereof. Refractory tumors also encompass tumors that appear to be inhibited by treatment with such agents, but recur up to five years, sometimes up to ten years or longer after treatment is discontinued. The tumors can express EGFR at normal levels or they can overexpress EGFR at levels, for example, that are at least 10, 100, or 1000 times normal levels.

Examples of tumors that express EGFR and can be treated by the modified anti-EGFR antibodies, and fragments thereof, provided herein include carcinomas, gliomas, sarcomas (including liposarcoma), adenocarcinomas, adenosarcomas, and adenomas. Such tumors can occur in virtually all parts of the body, including, for example, breast, heart, lung, small intestine, colon, spleen, kidney, bladder, head and neck, ovary, prostate, brain, pancreas, skin, bone, bone marrow, blood, thymus, uterus, testicles, cervix or liver.

Exemplary tumors that can be treated by the modified anti-EGFR antibodies, and fragments thereof, provided herein are those that overexpress EGFR. Some tumors observed to overexpress EGFR that can be treated include, but are not limited to, colorectal and head and neck tumors, especially squamous cell carcinoma of the head and neck, brain tumors such as glioblastomas, and tumors of the lung, breast, pancreas, esophagus, bladder, kidney, ovary, cervix, and prostate.

Other examples of tumors that can be treated by the anti-EGFR antibodies, and antibody fragments thereof, provided herein include Kaposi's sarcoma, CNS neoplasms, neuroblastomas, capillary hemangioblastomas, meningiomas and cerebral metastases, melanoma, gastrointestinal and renal carcinomas and sarcomas, rhabdomyosarcoma, glioblastoma (such as glioblastoma multiforme) and leiomyosarcoma. Examples of cancer that can express EGFR include, but are not limited to, lymphoma, blastoma, neuroendocrine tumors, mesothelioma, schwannoma, meningioma, melanoma, and leukemia or lymphoid malignancies. Examples of such cancers include hematologic malignancies, such as Hodgkin's lymphoma; non-Hodgkin's lymphomas (Burkitt's lymphoma, small lymphocytic lymphoma/chronic lymphocytic leukemia, mycosis fungoides, mantle cell lymphoma, follicular lymphoma, diffuse large B-cell lymphoma, marginal zone lymphoma, hairy cell leukemia and lymphoplasmacytic leukemia), tumors of lymphocyte precursor cells, including B-cell acute lymphoblastic leukemia/lymphoma, and T-cell acute lymphoblastic leukemia/lymphoma, thymoma, tumors of the mature T and NK cells, including peripheral T-cell leukemias, adult T-cell leukemia/T-cell lymphomas and large granular lymphocytic leukemia, Langerhans cell histocytosis, myeloid neoplasias such as acute myelogenous leukemias, including AML with maturation, AML without differentiation, acute promyelocytic leukemia, acute myelomonocytic leukemia, and acute monocytic leukemias, myelodysplastic syndromes, and chronic myeloproliferative disorders, including chronic myelogenous leukemia; tumors of the central nervous system such as glioma, glioblastoma, neuroblastoma, astrocytoma, medulloblastoma, ependymoma, and retinoblastoma; solid tumors of the head and neck (e.g., nasopharyngeal cancer, salivary gland carcinoma, and esophageal cancer), lung (e.g., small-cell lung cancer, non-small cell lung cancer, adenocarcinoma of the lung and squamous carcinoma of the lung), digestive system (e.g., gastric or stomach cancer including gastrointestinal cancer, cancer of the bile duct or biliary tract, colon cancer, rectal cancer, colorectal cancer, and anal carcinoma), reproductive system (e.g., testicular, penile, or prostate cancer, uterine, vaginal, vulval, cervical, ovarian, and endometrial cancer), skin (e.g., melanoma, basal cell carcinoma, squamous cell cancer, actinic keratosis), liver (e.g., liver cancer, hepatic carcinoma, hepatocellular cancer, and hepatoma), bone (e.g., osteoclastoma, and osteolytic bone cancers) additional tissues and organs (e.g., pancreatic cancer, bladder cancer, kidney or renal cancer, thyroid cancer, breast cancer, cancer of the peritoneum, and Kaposi's sarcoma), and tumors of the vascular system (e.g., angiosarcoma and hemangiopericytoma).

b. Non-Cancer Hyperproliferative Diseases

Modified anti-EGFR antibodies, and antibody fragments thereof, provided herein can be used to treat a non-cancer hyperproliferative disease in a subject. EGFR is a critical pathway element in signalling from G-protein-coupled receptors (GPCRs), cytokines, receptor tyrosine kinases and integrins to a variety of cellular responses such as mitogen activated protein kinase activation, gene transcription and proliferation. Ligand binding to EGFR can induce autophosphorylation of cytoplasmic tyrosine residues, which can initiate cellular pathways leading to cellular proliferation. Overexpression and/or overstimulation can result in hyperproliferation. For example, the EGFR vIII mutation causes the EGFR receptor to have a constitutively active kinase function and stimulate cellular proliferation. It is known in the art that anti-EGFR antibodies can treat non-cancer hyperproliferative disorders. For example, Menetrier's disease, a rare premalignant, non-cancerous, hyperproliferative disorder of the stomach, can be treated with cetuximab (Fiske et al. (2009) Sci Trasl. Med. 1(8): 8ra18; Myers et al. (2012)Mol. Cell. Proteomics 11:10.1074/mcp.M111.015222, 1-15).

Examples of hyperproliferative diseases that can be treated by the anti-EGFR antibodies provided herein include any hyperproliferative diseases that can be treated by administration of an anti-EGFR antibody and include, for example, psoriasis, actinic keratoses, and seborrheic keratoses, warts, keloid scars, and eczema. Also included are hyperproliferative diseases caused by virus infections, such as papilloma virus infection. Different types of psoriasis can display characteristics such as pus-like blisters (pustular psoriasis), severe sloughing of the skin I (erythrodermic psoriasis), drop-like dots (guttae psoriasis) and smooth inflamed lesions (inverse psoriasis). It is understood that treatment of psoriasis includes treatment of all types of psoriasis (e.g., psoriasis vulgaris, psoriasis pustulosa, erythrodermic psoriasis, psoriasis arthropathica, parapsoriasis, palmoplantar pustulosis).

c. Autoimmune Diseases or Disorders

Modified anti-EGFR antibodies, and antibody fragments thereof, provided herein can be used to treat autoimmune diseases or disorders. Examples of autoimmune diseases or disorders that can be treated with the anti-EGFR antibodies described herein include, but are not limited to, allogenic islet graft rejection, alopecia areata, ankylosing spondylitis, antiphospholipid syndrome, autoimmune Addison's disease, antineutrophil cytoplasmic autoantibodies (AN CA), autoimmune diseases of the adrenal gland, autoimmune hemolytic anemia, autoimmune hepatitis, autoimmune myocarditis, autoimmune neutropenia, autoimmune oophoritis and orchitis, autoimmune thrombocytopenia, autoimmune urticaria, Behcet's disease, bullous pemphigoid, cardiomyopathy, Castleman's syndrome, celiac spruce-dermatitis, chronic fatigue immune dysfunction syndrome, chronic inflammatory demyelinating polyneuropathy, Churg-Strauss syndrome, cicatricial pemphigoid, CREST syndrome, cold agglutinin disease, Crohn's disease, dermatomyositis, discoid lupus, essential mixed cryoglobulinemia, factor VIII deficiency, fibromyalgia-fibromyositis, glomerulonephritis, Grave's disease, Guillain-Barre, Goodpasture's syndrome, graft-versus-host disease (GVHD), Hashimoto's thyroiditis, hemophilia A, idiopathic pulmonary fibrosis, idiopathic thrombocytopenia purpura (ITP), IgA neuropathy, IgM polyneuropathies, immune mediated thrombocytopenia, juvenile arthritis, Kawasaki's disease, lichen planus, lupus erythematosus, Meniere's disease, mixed connective tissue disease, multiple sclerosis, type 1 diabetes mellitus, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Reynaud's phenomenon, Reiter's syndrome, rheumatoid arthritis, sarcoidosis, scleroderma, Sjögren's syndrome, solid organ transplant rejection, stiff-man syndrome, systemic lupus erythematosus, Takayasu arteritis, temporal arteritis/giant cell arteritis, thrombotic thrombocytopenia purpura, ulcerative colitis, uveitis, vasculitides such as dermatitis herpetiformis vasculitis, vitiligo, and Wegner's granulomatosis.

d. Inflammatory Disorders

Modified anti-EGFR antibodies, and antibody fragments thereof, provided herein can be used to treat inflammatory diseases or disorders. Inflammatory disorders that can be treated by the modified anti-EGFR antibodies provided herein include but are not limited to acute respiratory distress syndrome (ARDS), acute septic arthritis, allergic encephalomyelitis, allergic rhinitis, allergic vasculitis, allergy, asthma, atherosclerosis, chronic inflammation due to chronic bacterial or viral infections, chronic obstructive pulmonary disease (COPD), coronary artery disease, encephalitis, inflammatory bowel disease, inflammatory osteolysis, inflammation associated with acute and delayed hypersensitivity reactions, inflammation associated with tumors, peripheral nerve injury or demyelinating diseases, inflammation associated with tissue trauma such as burns and ischemia, inflammation due to meningitis, multiple organ injury syndrome, pulmonary fibrosis, sepsis and septic shock, Stevens-Johnson syndrome, undifferentiated arthropathy, and undifferentiated spondyloarthropathy.

e. Infectious Diseases

Modified anti-EGFR antibodies, and antibody fragments thereof, provided herein can be used to treat infectious diseases. Infectious diseases that can be treated by the anti-EGFR antibodies described herein include but are not limited to diseases caused by pathogens such as viruses, bacteria, fungi, protozoa, and parasites. Infectious diseases can be caused by viruses including adenovirus, cytomegalovirus, dengue, Epstein-Barr, hanta, hepatitis A, hepatitis B, hepatitis C, herpes simplex type I, herpes simplex type II, human immunodeficiency virus, (HIV), human papilloma virus (HPV), influenza, measles, mumps, papova virus, polio, respiratory syncytial virus, rinderpest, rhinovirus, rotavirus, rubella, SARS virus, smallpox and viral meningitis. Infectious diseases can also be caused by bacteria including Bacillus anthracia, Borrelia burgdorferi, Campylobacter jejuni, Chlamydia trachomatis, Clostridium botulinum, Clostridium tetani, Diphtheria, E. coli, Legionella, Helicobacter pylori, Mycobacterium rickettsia, Mycoplasma Neisseria, Pertussis, Pseudomonas aeruginosa, S. pneumonia, Streptococcus, Staphylococcus, Vibrio cholerae and Yersinia pestis. Infectious diseases can also be caused by fungi such as Aspergillus fumigatus, Blastomyces dermatitidis, Candida albicans, Coccidioides immitis, Cryptococcus neoformans, Histoplasma capsulatum and Penicillium marneffei. Infectious diseases can also be caused by protozoa and parasites such as chlamydia, kokzidiose, leishmania, malaria, rickettsia, and trypanosoma.

f. Other Diseases and Conditions

Modified anti-EGFR antibodies, and antibody fragments thereof, provided herein can be used to treat other diseases and conditions associated with expression of EGFR and/or for which exiting anti-EGFR antibodies, such as Cetuximab, are known to treat. Other diseases and conditions that can be treated by the anti-EGFR antibodies described herein include but are not limited to heart conditions such as congestive heart failure (CHF), myocarditis and other conditions of the myocardium; skin conditions such as rosacea, acne, and eczema; bone and tooth conditions such as bone loss, osteoporosis, Paget's disease, Langerhans' cell histiocytosis, periodontal disease, disuse osteopenia, osteomalacia, monostotic fibrous dysplasia, polyostotic fibrous dysplasia, bone metastasis, bone pain management, humoral malignant hypercalcemia, periodontal reconstruction, spinal cord injury, and bone fractures; metabolic conditions such as Gaucher's disease; endocrine conditions such as Cushing's syndrome; and neurological conditions.

2. Subjects for Therapy

A subject or candidate for therapy with a modified anti-EGFR antibody provided herein includes, but is not limited to, a subject, such as a human patient, that has a disease or condition that can be treated by administration of an anti-EGFR antibody, such as diseases or conditions described herein or known in the art.

a. Selection of Subjects Overexpressing EGFR

In some examples, subjects or candidates for therapy are tested for evidence of positive EGFR expression using methods known in the art, such as for example Western blotting (WB) of membrane-bound protein and/or total homogenates, and immunohistochemistry (IHC) on tissue microarrays. In addition, phosphorylated EGFR (pEGFR) can be measured by Western blot (see, e.g., Thariat et al. (2012) Clin. Cancer Res. 18:1313). EGFR assessment can be evaluated using, for example, the EGFR PHARMDX scoring guidelines (Dako, Glostrup, Denmark). EGFR expression can be evaluated on sections that include the deepest region of tumor invasion, which can contain the greatest density of EGFR-positive cells. Such methods are within the ability of the skilled artisan (see, e.g., Ervin-Haynes et al. (2006) J. Clin. Oncol. ASCO Annual Meeting Proceedings Part I. Vol. 24, No. 18S (June 20 Supplement)13000; Goldstein and Armin (2001) Cancer 92(5):1331-1346; Bibeau et al. (2006) Virchows Arch. 449(3):281-287).

b. Selection of Subjects Exhibiting EGFR-Associated Polymorphism

In some examples, subjects or candidates for therapy are screened for one or more polymorphisms in order to predict the efficacy of the anti-EGFR antibodies provided herein. A number of the receptors that can interact with anti-EGFR antibodies, such as the modified

EGFR antibodies provided herein, are polymorphic in the human population. For a given patient or population of patients, the efficacy of the modified anti-EGFR antibodies provided herein can be affected by the presence or absence of specific polymorphisms in proteins.

For example, FcγRIIIa is polymorphic at position 158, which is commonly either V (high affinity) or F (low affinity). Patients with the V/V homozygous genotype mount a stronger natural killer (NK) response and are observed to have a better clinical response to treatment with the anti-CD20 antibody Rituxan® (rituximab), (Dall'Ozzo et. al. (2004) Cancer Res. 64:4664-4669). Additional polymorphisms include but are not limited to FcγRIIa R131 or H131, and such polymorphisms are known to either increase or decrease Fc binding and subsequent biological activity, depending on the polymorphism.

In some examples, subjects or candidates for therapy are screened for one or more polymorphisms in order to predict the efficacy of the anti-EGFR antibodies provided herein. Such methods are within the ability of the skilled artisan. This information can be used, for example, to select patients to include or exclude from clinical trials or, post-approval, to provide guidance to physicians and patients regarding appropriate dosages and treatment options. For example, in patients that are homozygous or heterozygous for FcγRIIIa 158F antibody drugs, such as the anti-CD20 mAb Rituximab, can have decreased efficacy (Carton 2002 Blood 99: 754-758; Weng 2003 J. Clin. Oncol. 21:3940-3947); such patients can show a much better clinical response to the modified anti-EGFR antibodies provided herein.

c. Identifying Subjects Exhibiting Anti-EGFR-Associated Side Effects

In some examples, a subject or candidate for therapy with a modified anti-EGFR antibody provided herein, includes, but is not limited to, a subject, such as a human patient, that has experienced one more side effects resulting from administration of an anti-EGFR antibody, such as any anti-EGFR antibody known in the art. Administration of an anti-EGFR provided herein to the subject in place of the anti-EGFR antibody therapy that caused the side effect(s) can result in comparable or improved therapeutic efficacy, while resulting in reduced or lessened side effect(s).

The dosage regimen, including dosage amount and frequency of administration, of the anti-EGFR provided herein can be the same or different than the previous anti-EGFR antibody therapy. In some cases, the dosage amount can be increased or decreased. It is within the skill of the practicing physician to determine the dosage regimen based on factors such as the particular subject being treated, the nature of the disease or condition, the nature of the existing symptoms or side effects and the particular modified anti-EGFR antibody provided herein that is to be administered.

As discussed elsewhere herein, EGFR is expressed in many normal human tissues (Lacouture, and Melosky (2007) Skin Therapy Lett. 12, 1-5). Therefore, administration of many therapeutic anti-EGFR antibodies, such as Cetuximab, can result in undesirable reactions. Such side effects are well-known to one of skill in the art and can be assessed or identified. Methods to identify side effects caused by an anti-EGFR antibody therapeutic include any methods described herein, such as patient interview, patient examination and blood tests. Side effects that can be assessed include any side effects that are known to one of skill in the art to be associated with administration of an anti-EGFR antibody, including any side effects described herein, such as, for example, a side effect associated with administration of Cetuximab.

For example, side effects of Cetuximab include any described herein and/or known to one of skill in the art, including symptomatic hypomagnesemia, paronychia, fever, dermatologic toxicity, papulopustular rash of the face and upper trunk, hair growth abnormalities, loss of scalp hair, increased growth of facial hair and eyelashes, dry and itchy skin, and periungual inflammation with tenderness (Eng (2009) Nat. Rev. 6:207-218; Schrag et al. (2005) J. Natl. Cancer Inst. 97(16):1221-1224; Lacouture and Melosky (2007) Skin Therapy Lett. 12:1-5). In some examples, the side effects of Cetuximab include dermatological toxicities, including papulopustular eruption, dry skin, pruritus, ocular and nail changes, acneiform skin reaction, acneiform rash, acneiform follicular rash, acne-like rash, maculopapular skin rash, monomorphic pustular lesions, papulopustular reaction (Lacouture and Melosky (2007) Skin Therapy Lett. 12:1-5).

The side effects can be triggered by external events and/or can develop over time. For example, skin rashes can be triggered by sun exposure and can develop in stages, such as sensory disturbance, erythema, and edema (for example, week 1); papulopustular eruption (for example, week 2); and crusting (for example, week 4). If the rash is treated successfully, erythema and dry skin can be seen in areas previously affected by the papulopustular eruption (for example, weeks 4-6). Other dermatological toxicities that can be associated with administration of an anti-EGFR antibody, such as Cetuximab include pruritus, erythema and paronychial inflammation (Lacouture, and Melosky (2007) Skin Therapy Lett. 12, 1-5). For example, Cetuximab elicits an immune response in about 5% of patients. Such an immune response can result in an immune complex-mediated clearance of the antibodies or fragments from the circulation, and make repeated administration unsuitable for therapy, thereby reducing the therapeutic benefit to the patient and limiting the re-administration of the antibody.

In some examples, the severity of side effects can be evaluated according to the National Cancer Institute Common Terminology Criteria for Adverse Events (CTCAE) v4.0, which sets forth criteria for grading the severity for side effects. The CTCAE includes Grades 1 through 5 that set forth unique clinical descriptions of severity for each adverse effect. Under the general guidelines of the CTCAE, Grade 1 adverse events are mild, asymptomatic or mild symptoms, clinical or diagnostic observations only; and intervention is not indicated. Grade 2 adverse events are moderate, minimal, local or noninvasive intervention indicated, limiting age-appropriate instrumental Activities of Daily Living (ADL). Grade 3 adverse events are severe or medically significant but not immediately life-threatening, with hospitalization or prolongation of hospitalization indicated, disabling and limiting self-care ADL. Grade 4 adverse events are life-threatening consequences, and urgent intervention is indicated. Grade 5 adverse events are classified as death related to the adverse event(s). Thus, for example, administering an anti-EGFR antibody provided herein in a subject identified as having a particular grade of side effects can result in a reduction of side effects is characterized by a reduction in the grade of the side effect as classified under the CTCAE v4.0. In some examples, reduction of side effects is characterized by a reduction in the severity of the symptoms associated with the side effect, including any symptoms described herein or known to one of skill in the art.

Other methods to identify patients that exhibit a side effect of an anti-EGFR antibody are known to one of skill in the art, and include quality-of-life questionnaires (e.g., Jonker et al. (2007) N. Engl. J. Med. 357:2040-2048). Examples of side effects of anti-EGFR antibodies, and methods known to the skilled artisan to identify the severity of side effects, are described below. These side effects are exemplary and not meant to be limiting. It is understood that any side effects known in the art or described herein that are associated with administration of an anti-EGFR antibody, such as Cetuximab, can be identified in a subject, whereby the subject can then be treated with a modified anti-EGFR antibody, provided herein so that such side effects are not further exacerbated and/or are reduced.

### i. Skin Toxicities

In human skin, EGFR is expressed in basal keratinocytes and can stimulate epidermal growth, inhibit differentiation, and accelerate wound healing (Lacouture, and Melosky (2007) Skin Therapy Lett. 12:1-5; Nanney et al. (1996) J. Invest. Dermatol 94(6):742-748). Therefore anti-EGFR antibodies that interact with and inhibit EGFR expressed by basal keratinocytes can impair growth and migration of keratinocytes, and result in inflammatory chemokine expression. These effects can lead to inflammatory cell recruitment and subsequent cutaneous injury, which can result in side effects, such as side effects described herein. The pH of the skin basal layer environment is neutral (e.g., at or about pH 7.0-7.4). Therefore, modified anti-EGFR antibodies, that have increased activity at low pH than at neutral pH, such as the anti-EGFR antibodies provided herein, can have decreased skin toxicity and decreased side effects. Examples of side effects resulting from EGFR inhibition in the skin, and methods of identification and classification thereof, are described below.

Papulopustular rash and acneiform rash, which are characterized by an eruption consisting of papules (a small, raised pimple) and pustules (a small pus filled blister), typically appearing in face, scalp, and upper chest and back. Unlike acne, papulopustular rash does not present with whiteheads or blackheads, and can be symptomatic, with itchy or tender lesions (CTCAE v. 4.03, U.S. Department of Health and Human Services, published Jun. 14, 2010). Papulopustular rash and acneiform rash can be identified and classified by examination of the patient and/or by clinical interview. Grade 1 papulopustular rash or acneiform rash is classified as papules and/or pustules covering <10% Body Surface Area (BSA), which can be associated with symptoms of pruritus or tenderness. Grade 2 papulopustular rash or acneiform rash is classified as papules and/or pustules covering 10-30% BSA, which can be associated with symptoms of pruritus or tenderness; associated with psychosocial impact; limiting instrumental activities of daily living (ADL). Grade 3 papulopustular rash or acneiform rash is classified as papules and/or pustules covering >30% BSA, which can be associated with symptoms of pruritus or tenderness; limiting self-care ADL; and can be associated with local superinfection with oral antibiotics indicated. Grade 4 papulopustular rash or acneiform rash is classified as papules and/or pustules covering any percent BSA, which can be associated with symptoms of pruritus or tenderness and are associated with extensive superinfection with IV antibiotics indicated; and life-threatening consequences. Grade 5 papulopustular rash or acneiform rash is classified as resulting in death (CTCAE v. 4.03, U.S. Department of Health and Human Services, published Jun. 14, 2010; Schrag J. Natl. Cancer. Inst. 97(16):1221-1224).

An example of a side effect of an anti-EGFR antibody, such as Cetuximab, is dry skin, which is a disorder characterized by flaky and dull skin; fine pores, and papery thin skin texture. Dry skin can be identified and classified by examination of the patient and/or by clinical interview. Grade 1 dry skin is classified as covering <10% BSA and no associated erythema or pruritus. Grade 2 dry skin is classified as covering 10%-30% BSA, associated with erythema or pruritus and limiting instrumental ADL. Grade 3 dry skin is classified as covering >30% BSA, associated with pruritus and limiting self-care ADL (CTCAE v. 4.03, U.S. Department of Health and Human Services, published Jun. 14, 2010; Schrag J. Natl. Cancer. Inst. 97(16):1221-1224).

Skin hyperpigmentation is a side effect characterized by darkening of the skin due to excessive melanin deposition. Skin hyperpigmentation can be identified and classified by examination of the patient and/or by clinical interview. Grade 1 skin hyperpigmentation is classified as hyperpigmentation covering <10% BSA, no psychosocial impact. Grade 2 skin hyperpigmentation is classified as hyperpigmentation covering >10% BSA, and associated with psychosocial impact. (CTCAE v. 4.03, U.S. Department of Health and Human Services, published Jun. 14, 2010; Schrag J. Natl. Cancer. Inst. 97(16):1221-1224).

Pruritus is a side effect characterized by an intense itching sensation. Pruritus can be evaluated by patient examination and/or clinical interview. Grade 1 pruritus is classified as mild or localized itching, and topical intervention is indicated. Symptoms of grade 2 pruritus include intense or widespread itching, intermittent itching, skin changes from scratching (e.g., edema, papulation, excoriations, lichenification, oozing/crusts), limiting instrumental ADL, and oral intervention can be indicated. Symptoms of grade 3 pruritus include intense, widespread and/or constant itching, limiting self-care ADL or sleep, and oral corticosteroid or immunosuppressive therapy can be indicated (CTCAE v. 4.03, U.S. Department of Health and Human Services, published Jun. 14, 2010; Schrag J. Natl. Cancer. Inst. 97(16):1221-1224).

Paronychia is a side effect characterized by an infectious process involving the soft tissues around the nail. Paronychia can be evaluated by patient examination and/or clinical interview. Grade 1 paronychia is classified as including symptoms of nail fold edema or erythema and disruption of the cuticle. Symptoms of grade 2 paronychia can include localized intervention indicated, oral intervention indicated (e.g., antibiotic, antifungal, antiviral), nail fold edema or erythema with pain, discharge or nail plate separation and limiting instrumental ADL. Symptoms of grade 3 paronychia can include limiting self-care ADL, with surgical intervention or IV antibiotics indicated.

### ii. Hypomagnesemia

EGFR is highly expressed in the kidney, particularly in the ascending limb of the loop of Henle where 70% of filtered magnesium is reabsorbed. Therefore, antibodies that interact with EGFR can interfere with magnesium transport. Hypomagnesemia, a low concentration of magnesium in the blood, can be a side effect of administration of an anti-EGFR antibody. In one study, five percent of patients receiving cetuximab therapy exhibited grade 3 or 4 hypomagnesemia.

The loop of Henle has a neutral pH (e.g., pH 6.9-7.4) (Dieleman et al. (2001) J. Acquir Immune Defic Syndr. 28(1):9-13; Dantzler et al. (2000) Pflugers Arch. 440(1):140-148.). Therefore, modified anti-EGFR antibodies that have increased activity at low pH than at neutral pH, such as the modified anti-EGFR antibodies provided herein, can have decreased hypomagnesemia.

Hypomagnesemia can be diagnosed and/or assessed by measurement of serum magnesium levels. For example, the CTCAE classifies Grade 1 hypomagnesemia as a serum magnesium concentration of <Lower Limit of Normal (LLN)—1.2 mg/dL; Grade 2 hypomagnesemia as 1.2-0.9 mg/dL serum magnesium; Grade 3 hypomagnesemia as <0.9-0.7 mg/dL serum magnesium, Grade 4 hypomagnesemia as <0.7 mg/dL serum magnesium and can be accompanied by life-threatening consequences and Grade 5 hypomagnesemia results in death. In addition, symptoms of hypomagnesemia are known to the skilled artisan and include fatigue, paresthesias and hypocalcemia. (CTCAE v. 4.03, U.S. Department of Health and Human Services, published Jun. 14, 2010; Schrag J. Natl. Cancer. Inst. 97(16):1221-1224).

d. Other Methods of Selecting or Identifying Subjects for Treatment

Other methods of screening candidates for therapy known in the art are contemplated. For example, Kirsten rat sarcoma viral oncogene homolog (KRAS) mutation status has recently been shown to be predictive of response to cetuximab therapy in colorectal cancer (Van Cutsem et al. (2008) J. Clin. Oncol 26 (May 20 suppl): Abstract 2). KRAS is a GTPase with a role in a number of signal transduction pathways. Mutations in the gene which encodes KRAS, present in over 25% of colorectal cancers, is predictive of the success of EGFR-inhibiting drugs. Expression of the mutated KRAS gene results in a diminished response to EGFR-inhibitor therapy. KRAS mutations can be detected by commercially available laboratory diagnostics.

3. Dosages

A therapeutically effective amount of an anti-EGFR antibody or antibody fragment can be administered for treatment of any of the diseases or conditions provided herein or known to the skilled artisan. Such dosages can be empirically determined by one of skill in the art, such as the treating physician. In some examples, the administered dosages are based on reference to dosage amounts of known anti-EGFR antibodies, such as Cetuximab, for a particular disease or condition. The therapeutically effective concentration of a modified anti-EGFR antibody, provided herein can be determined empirically by testing the anti-EGFR antibodies in known in vitro and in vivo systems such as by using the assays provided herein or known in the art.

An effective amount of anti-EGFR antibody to be administered therapeutically will depend, for example, upon the therapeutic objectives, the route of administration, and the condition of the patient. In addition, the attending physician can take into consideration various factors known to modify the action of drugs, including severity and type of disease, patient's health, body weight, sex, diet, time and route of administration, other medications and other relevant clinical factors. In addition, the therapist can consider the incidence and severity of side effects, such as side effects described herein or known in the art. Accordingly, the therapist can titer the dosage of the antibody or antigen-binding fragment thereof and modify the route of administration as required to obtain the optimal therapeutic effect and minimize undesirable side effects. The clinician can administer the antibody until a dosage is reached that achieves the desired effect. The progress of this therapy can be monitored by conventional assays described herein or known in the art. The dose of the modified anti-EGFR antibody can be varied to identify the optimal or minimal dose required to achieve activity while reducing or eliminating side effects.

Generally, the dosage ranges for the administration of the modified anti-EGFR antibodies provided herein are those large enough to produce the desired therapeutic effect in which the symptom(s) of the condition responsive to treatment with an anti-EGFR antibody are ameliorated. Generally, the dosage will vary with the age, condition, sex and the extent of the disease in the patient and can be determined by one of skill in the art. In some examples, the dosage is not so large as to cause adverse side effects. The dosage can be adjusted by the individual physician in the event of the appearance of any adverse side effect.

Exemplary dosages include, but are not limited to, about or 0.1 mg/kg to 100 mg/kg, such as at least about or about 0.1 mg/kg, about or 0.15 mg/kg, about or 0.2 mg/kg, about or 0.25 mg/kg, about or 0.30 mg/kg, about or 0.35 mg/kg, about or 0.40 mg/kg, about or 0.45 mg/kg, about or 0.5 mg/kg, about or 0.55 mg·kg, about or 0.6 mg/kg, about or 0.7 mg/kg, about or 0.8 mg/kg, about or 0.9 mg/kg, about or 1.0 mg/kg, about or 1.1 mg/kg, about or 1.2 mg/kg, about or 1.3 mg/kg, about or 1.4 mg/kg, about or 1.5 mg/kg, about or 1.6 mg/kg, about or 1.7 mg/kg, about or 1.8 mg/kg, about or 1.9 mg/kg, about or 2 mg/kg, about or 2.5 mg/kg, about or 3 mg/kg, about or 3.5 mg/kg, about or 4 mg/kg, about or 4.5 mg/kg, about or 5 mg/kg, about or 5.5 mg/kg, about or 6 mg/kg, about or 6.5 mg/kg, about or 7 mg/kg, about or 7.5 mg/kg, about or 8 mg/kg, about or 8.5 mg/kg, about or 9 mg/kg, about or 9.5 mg/kg, about or 10 mg/kg, about or 11 mg/kg, about or 12 mg/kg, about or 13 mg/kg, about or 14 mg/kg, about or 15 mg/kg, about or 16 mg/kg, about or 17 mg/kg, about or 18 mg/kg, about or 19 mg/kg, about or 20 mg/kg, about or 21 mg/kg, about or 22 mg/kg, about or 23 mg/kg, about or 24 mg/kg, about or 25 mg/kg, about or 30 mg/kg, about or 40 mg/kg, about or 50 mg/kg, about or 60 mg/kg, about or 70 mg/kg, about or 80 mg/kg, about or 90 mg/kg, about or 100 mg/kg or more.

In some examples, exemplary dosages include, but are not limited to, about or 0.01 mg/m² to about or 800 mg/m², such as for example, at least about or about or 0.01 mg/m², about or 0.1 mg/m², about or 0.5 mg/m², about or 1 mg/m², about or 5 mg/m², about or 10 mg/m², about or 15 mg/m², about or 20 mg/m², about or 25 mg/m², about or 30 mg/m², about or 35 mg/m², about or 40 mg/m², about or 45 mg/m², about or 50 mg/m², about or 100 mg/m², about or 150 mg/m², about or 200 mg/m², about or 250 mg/m², about or 300 mg/m², about or 400 mg/m², about or 500 mg/m², about or 600 mg/m² and about or 700 mg/m². It is understood that one of skill in the art can recognize and convert dosages between units of mg/kg and mg/m² (see, e.g., Michael J. Derelanko, TOXICOLOGIST'S POCKET HANDBOOK, CRC Press, p. 16 (2000)).

For treatment of a disease or condition, the dosage of the anti-EGFR antibodies can vary depending on the type and severity of the disease. The anti-EGFR antibodies can be administered in a single dose, in multiple separate administrations, or by continuous infusion. For repeated administrations over several days or longer, depending on the condition, the treatment can be repeated until a desired suppression of disease symptoms occurs or the desired improvement in the patient's condition is achieved. Repeated administrations can include increased or decreased amounts of the anti-EGFR antibody depending on the progress of the treatment. For example, an initial loading dose can be larger than a maintenance dose. In some examples, the initial loading dose is 400 mg/m², and the maintenance dose is 250 mg/m².

Other dosage regimens also are contemplated. For example, the dosage regimen can be varied. Modified anti-EGFR antibodies that are associated with reduced side effects can be used at higher dosing regimens. In addition, anti-EGFR antibodies that have increased activity in diseased tissues can be used at lower dosing regimens. Methods of determining efficacy of the administered modified anti-EGFR antibodies described herein, are known to one of skill in the art and exemplary methods are described herein, and can be utilized to empirically determine an appropriate dosage regimen. The optimal quantity and spacing of individual dosages of an anti-EGFR antibody of the disclosure will be determined by the nature and extent of the condition being treated, the form, the route and site of administration, and the age and condition of the particular subject being treated, and a physician can determine appropriate dosages to be used. This dosage can be repeated as often as appropriate. If side effects develop, the amount and/or frequency of the dosage can be altered or reduced, in accordance with normal clinical practice. Such studies and practices are within the level of one of skill in the art.

In some examples, the anti-EGFR antibodies are administered one time, two times, three times, four times, five times, six times, seven times, eight times, nine times, ten times or more per day or over several days. In some examples, the anti-EGFR antibodies are administered in a sequence of two or more administrations, where the administrations are separated by a selected time period. In some examples, the selected time period is at least or about 1 day, 2 days, 3 days, 4 days, 5 days, 6 days, 1 week, 2 weeks, 3 weeks, 1 month, 2 months, or 3 months.

Side effects of a particular dosage or dosage regimen also can be assessed, for example, by any methods described herein or known in the art, following administration of one or more doses of the anti-EGFR antibody thereof. Dosage amounts and/or frequency of administration can be modified depending on the type and severity of the side effect(s).

As will be understood by one of skill in the art, the optimal treatment regimen will vary and it is within the scope of the treatment methods to evaluate the status of the disease under treatment and the general health of the patient prior to, and following one or more cycles of therapy in order to determine the optimal therapeutic dosage and frequency of administration. It is to be further understood that for any particular subject, specific dosage regimens can be adjusted over time according to the individual need and the professional judgment of the person administering or supervising the administration of the pharmaceutical formulations, and that the dosages set forth herein are exemplary only and are not intended to limit the scope thereof. The amount of an anti-EGFR antibody to be administered for the treatment of a disease or condition, such as a disease or condition described herein, can be determined by standard clinical techniques described herein or known in the art. In addition, in vitro assays and animal models can be employed to help identify optimal dosage ranges. Such assays can provide dosages ranges that can be extrapolated to administration to subjects, such as humans. Methods of identifying optimal dosage ranges based on animal models are well-known by those of skill in the art, and examples are described herein.

4. Routes of Administration

The anti-EGFR antibodies provided herein can be administered to a subject by any method known in the art for the administration of polypeptides, including for example systemic or local administration. The anti-EGFR antibodies can be administered by routes, such as parenteral (e.g., intradermal, intramuscular, intraperitoneal, intravenous, subcutaneous, or intracavity), topical, epidural, or mucosal (e.g., intranasal, oral, vaginally, vulvovaginal, esophageal, oroesophageal, bronchial, or pulmonary). The anti-EGFR antibodies can be administered externally to a subject, at the site of the disease for exertion of local or transdermal action. Compositions containing anti-EGFR antibodies or antigen-binding fragments can be administered by any convenient route, for example by infusion or bolus injection, by absorption through epithelial or mucocutaneous linings (e.g., oral mucosa, vaginal, rectal and intestinal mucosa). Compositions containing anti-EGFR antibodies or antigen-binding fragments can be administered together with other biologically active agents. In particular examples, the anti-EGFR antibodies are administered by infusion delivery, such as by infusion pump or syringe pump, and can be administered in combination with another therapeutic agent or as a monotherapy.

The method and/or route of administration can be altered to alleviate adverse side effects associated with administration of an anti-EGFR antibody provided herein. For example, if a patient experiences a mild or moderate (i.e., Grade 1 or 2) infusion reaction, the infusion rate can be reduced (e.g., reduced by 10%, 20%, 30%, 40%, 50%, 60%, 70%, 80%, 90% or more). If the patient experiences severe (i.e., Grade 3 or 4) infusion reactions, the infusion can be temporarily or permanently discontinued.

In some examples, if the subject experiences an adverse side effect, such as severe skin toxicity, for example severe acneform rash, treatment adjustments can be made. For example, after the occurrence of an adverse side effect, administration can be delayed, such as for 1 to 2 weeks or until the adverse side effect improves. In some examples, after additional occurrences of an adverse side effect, the dosage can be reduced. For example, if the dose is 250 mg/m², after the second occurrence of an adverse side effect, administration of the anti-EGFR antibody can be delayed for 1 to 2 weeks. If the side effect improves, administration of the anti-EGFR antibody can continue with the dose reduced to 250 mg/m². After the third occurrence of the side effect, administration of the anti-EGFR antibody can be delayed for 1 to 2 weeks. If the side effect improves, administration of the anti-EGFR antibody can continue with the dose reduced to 150 mg/m². After several occurrences of an adverse side effect, administration of the anti-EGFR antibody can be discontinued. In patients with mild or moderate skin toxicity, the skilled artisan can continue administration without dose modification. Such determinations are within the ability of the skilled artisan.

Appropriate methods for delivery, can be selected by one of skill in the art based on the properties of the dosage amount of the anti-EGFR antibody or the pharmaceutical composition containing the antibody or antigen-binding fragment thereof. Such properties include, but are not limited to, solubility, hygroscopicity, crystallization properties, melting point, density, viscosity, flow, stability and degradation profile.

5. Combination Therapies

The modified anti-EGFR antibodies provided herein can be administered before, after, or concomitantly with one or more other therapeutic regimens or agents. The skilled medical practitioner can determine empirically, or by considering the pharmacokinetics and modes of action of the agents, the appropriate dose or doses of each therapeutic regimen or agent, as well as the appropriate timings and methods of administration. The additional therapeutic regimens or agents can improve the efficacy or safety of the anti-EGFR antibody. In some examples, the additional therapeutic regimens or agents can treat the same disease or a comorbidity rather than to alter the action of the anti-EGFR antibody. In some examples, the additional therapeutic regimens or agents can ameliorate, reduce or eliminate one or more side effects known in the art or described herein that are associated with administration of an anti-EGFR antibody.

For example, an anti-EGFR antibody described herein can be administered with chemotherapy, radiation therapy, or both chemotherapy and radiation therapy. The modified anti-EGFR antibodies can be administered in combination with one or more other prophylactic or therapeutic agents, including but not limited to antibodies, cytotoxic agents, chemotherapeutic agents, cytokines, growth inhibitory agents, anti-hormonal agents, kinase inhibitors, anti-angiogenic agents, cardioprotectants, immunostimulatory agents, immunosuppressive agents, agents that promote proliferation of hematological cells, angiogenesis inhibitors, protein tyrosine kinase (PTK) inhibitors, additional anti-EGFR antibodies, FcγRIIb or other Fc receptor inhibitors, or other therapeutic agents.

The one or more additional agents can be administered simultaneously, sequentially or intermittently with the anti-EGFR antibody thereof. The agents can be co-administered with the anti-EGFR antibody thereof, for example, as part of the same pharmaceutical composition or same method of delivery. In some examples, the agents can be co-administered with the anti-EGFR antibody at the same time as the modified anti-EGFR antibody thereof, but by a different means of delivery. The agents also can be administered at a different time than administration of the anti-EGFR antibody thereof, but close enough in time to the administration of the anti-EGFR antibody to have a combined prophylactic or therapeutic effect. In some examples, the one or more additional agents are administered subsequent to or prior to the administration of the anti-EGFR antibody separated by a selected time period. In some examples, the time period is 1 day, 2 days, 3 days, 4 days, 5 days, 6 days, 1 week, 2 weeks, 3 weeks, 1 month, 2 months, or 3 months. In some examples, the one or more additional agents are administered multiple times and/or the anti-EGFR antibody provided herein is administered multiple times.

In some examples, a modified anti-EGFR antibody, provided herein is administered with one or more antibodies or antibody fragments. The anti-EGFR antibody can be administered with one or more other antibodies that have efficacy in treating the same disease or an additional comorbidity. For example, the one or more antibodies administered with the anti-EGFR antibody can be selected from among anti-cancer antibodies, antibodies to treat autoimmune or inflammatory disease, antibodies to treat transplant rejection, antibodies to treat graft-versus-host-disease (GVHD) and antibodies to treat infectious diseases. In some examples, two or more of the anti-EGFR antibodies provided herein are administered in combination.

Examples of anti-cancer antibodies that can be co-administered with an anti-EGFR antibody provided herein include, but are not limited to, anti-17-IA cell surface antigen antibodies such as Panorex® (edrecolomab); anti-4-1BB antibodies; anti-4Dc antibodies; anti-A33 antibodies such as A33 and CDP-833; anti-α1 integrin antibodies such as natalizumab; anti-α4β7 integrin antibodies such as LDP-02; anti-αVβ1 integrin antibodies such as F-200, M-200, and SJ-749; anti-αVβ3 integrin antibodies such as abciximab, CNTO-95, Mab-17E6, and Vitaxin®; anti-complement factor 5 (C5) antibodies such as 5G1.1; anti-CA125 antibodies such as OvaRex® (oregovomab); anti-CD3 antibodies such as Nuvion® (visilizumab) and Rexomab; anti-CD4 antibodies such as IDEC-151, MDX-CD4, OKT4A; anti-CD6 antibodies such as Oncolysin B and Oncolysin CD6; anti-CD7 antibodies such as HB2; anti-CD19 antibodies such as B43, MT-103, and Oncolysin B; anti-CD20 antibodies such as 2H7, 2H7.v16, 2H7.v114, 2H7.v115, Bexxar®(tositumomab), Rituxan® (rituximab), and Zevalin® (Ibritumomab tiuxetan); anti-CD22 antibodies such as Lymphocide® (epratuzumab); anti-CD23 antibodies such as IDEC-152; anti-CD25 antibodies such as basiliximab and Zenapax® (daclizumab); anti-CD30 antibodies such as AC10, MDX-060, and SGN-30; anti-CD33 antibodies such as Mylotarg® (gemtuzumab ozogamicin), Oncolysin M, and Smart M1 95; anti-CD38 antibodies; anti-CD40 antibodies such as SGN-40 and toralizumab; anti-CD40L antibodies such as 5c8, Antova®, and IDEC-131; anti-CD44 antibodies such as bivatuzumab; anti-CD46 antibodies; anti-CD52 antibodies such as Campath® (alemtuzumab); anti-CD55 antibodies such as SC-1; anti-CD56 antibodies such as huN901-DM1; anti-CD64 antibodies such as MDX-33; anti-CD66e antibodies such as XR-303; anti-CD74 antibodies such as IMMU-1 10; anti-CD80 antibodies such as galiximab and IDEC-1 14; anti-CD89 antibodies such as MDX-214; anti-CD123 antibodies; anti-CD138 antibodies such as B-B4-DM1; anti-CD146 antibodies such as AA-98; anti-CD148 antibodies; anti-CEA antibodies such as cT84.66, labetuzumab, and Pentacea®; anti-CTLA-4 antibodies such as MDX-101; anti-CXCR4 antibodies; anti-EGFR antibodies such as ABX-EGF, Erbitux® (cetuximab), IMC-C225, and Merck Mab 425; anti-EpCAM antibodies such as Crucell's anti-EpCAM, ING-1, and IS-IL-2; anti-ephrin B2/EphB4 antibodies; anti-Her2 antibodies such as Herceptin®), MDX-210; anti-FAP (fibroblast activation protein) antibodies such as sibrotuzumab; anti-ferritin antibodies such as NXT-211; anti-FGF-1 antibodies; anti-FGF-3 antibodies; anti-FGF-8 antibodies; anti-FGFR antibodies, anti-fibrin antibodies; anti-G250 antibodies such as WX-G250 and Rencarex®; anti-GD2 ganglioside antibodies such as EMD-273063 and TriGem; anti-GD3 ganglioside antibodies such as BEC2, KW-2871, and mitumomab; anti-gpIIb/IIIa antibodies such as ReoPro; anti-heparinase antibodies; anti-Her2/ErbB2 antibodies such as Herceptin® (trastuzumab), MDX-210, and pertuzumab; anti-HLA antibodies such as Oncolym®, Smart 1D10; anti-HM1.24 antibodies; anti-ICAM antibodies such as ICM3; anti-IgA receptor antibodies; anti-IGF-1 antibodies such as CP-751871 and EM-164; anti-IGF-1R antibodies such as IMC-A12; anti-IL-6 antibodies such as CNTO-328 and elsilimomab; anti-IL-15 antibodies such as HuMax®-IL15; anti-KDR antibodies; anti-laminin 5 antibodies; anti-Lewis Y antigen antibodies such as Hu3S193 and IGN-311; anti-MCAM antibodies; anti-Muc1 antibodies such as BravaRex and TriAb; anti-NCAM antibodies such as ERIC-1 and ICRT; anti-PEM antigen antibodies such as Theragyn and Therex; anti-PSA antibodies; anti-PSCA antibodies such as IG8; anti-Ptk antibodies; anti-PTN antibodies; anti-RANKL antibodies such as AMG-162; anti-RLIP76 antibodies; anti-SK-1 antigen antibodies such as Monopharm C; anti-STEAP antibodies; anti-TAG72 antibodies such as CC49-SCA and MDX-220; anti-TGF-β antibodies such as CAT-152; anti-TNF-α antibodies such as CDP571, CDP870, D2E7, Humira® (adalimumab), and Remicade® (infliximab); anti-TRAIL-R1 and TRAIL-R2 antibodies; anti-VE-cadherin-2 antibodies; and anti-VLA-4 antibodies such as Antegren®. Furthermore, anti-idiotype antibodies including but not limited to the GD3 epitope antibody BEC2 and the gp72 epitope antibody 105AD7, can be used. In addition, bispecific antibodies including but not limited to the anti-CD3/CD20 antibody Bi20 can be used.

Examples of antibodies that can treat autoimmune or inflammatory disease, transplant rejection, GVHD, that can be co-administered with a modified anti-EGFR antibody provided herein include, but are not limited to, anti-α4β7 integrin antibodies such as LDP-02, anti-beta2 integrin antibodies such as LDP-01, anti-complement (C5) antibodies such as 5G1.1, anti-CD2 antibodies such as BTI-322, MEDI-507, anti-CD3 antibodies such as OKT3, SMART anti-CD3, anti-CD4 antibodies such as IDEC-151, MDX-CD4, OKT4A, anti-CD11a antibodies, anti-CD14 antibodies such as IC14, anti-CD18 antibodies, anti-CD23 antibodies such as IDEC 152, anti-CD25 antibodies such as Zenapax, anti-CD40L antibodies such as 5c8, Antova, IDEC-131, anti-CD64 antibodies such as MDX-33, anti-CD80 antibodies such as IDEC-114, anti-CD147 antibodies such as ABX-CBL, anti-E-selectin antibodies such as CDP850, anti-gpIIb/IIIa antibodies such as ReoPro®/Abcixima, anti-ICAM-3 antibodies such as ICM3, anti-ICE antibodies such as VX-740, anti-FcγR1 antibodies such as MDX-33, anti-IgE antibodies such as rhuMAb-E25, anti-IL-4 antibodies such as SB-240683, anti-IL-5 antibodies such as SB-240563, SCH55700, anti-IL-8 antibodies such as ABX-IL8, anti-interferon gamma antibodies, and anti-TNFa antibodies such as CDP571, CDP870, D2E7, Infliximab, MAK-195F, anti-VLA-4 antibodies such as Antegren. Examples of other Fc-containing molecules that can be co-administered to treat autoimmune or inflammatory disease, transplant rejection and GVHD include, but are not limited to, the p75 TNF receptor/Fc fusion Enbrel® (etanercept) and Regeneron's IL-1 trap.

Examples of antibodies that can be co-administered to treat infectious diseases include, but are not limited to, anti-anthrax antibodies such as ABthrax, anti-CMV antibodies such as CytoGam and sevirumab, anti-cryptosporidium antibodies such as CryptoGAM, Sporidin-G, anti-helicobacter antibodies such as Pyloran, anti-hepatitis B antibodies such as HepeX-B, Nabi-HB, anti-HIV antibodies such as HRG-214, anti-RSV antibodies such as felvizumab, HNK-20, palivizumab, RespiGam, and anti-staphylococcus antibodies such as Aurexis, Aurograb, BSYX-A110, and SE-Mab.

In some examples, a modified anti-EGFR antibody described herein is administered with one or more molecules that compete for binding to one or more Fc receptors. For example, co-administering inhibitors of the inhibitory receptor FcγRIIb can result in increased effector function. Similarly, co-administering inhibitors of the activating receptors such as FcγRIIIa can minimize unwanted effector function. Fc receptor inhibitors include, but are not limited to, Fc molecules that are engineered to act as competitive inhibitors for binding to FcγRIIb, FcγRIIIa, or other Fc receptors, as well as other immunoglobulins and specifically the treatment called IVIg (intravenous immunoglobulin). In one embodiment, the inhibitor is administered and allowed to act before the anti-EGFR antibody is administered. An alternative way of achieving the effect of sequential dosing would be to provide an immediate release dosage form of the Fc receptor inhibitor and then a sustained release formulation of the anti-EGFR antibody. The immediate release and controlled release formulations could be administered separately or be combined into one unit dosage form.

In some examples, a modified anti-EGFR antibody described herein is administered with one or more chemotherapeutic agents. Examples of chemotherapeutic agents include but are not limited to alkylating agents such as thiotepa and cyclophosphamide (CYTOXAN®); alkyl sulfonates such as busulfan, improsulfan and piposulfan; androgens such as calusterone, dromostanolone propionate, epitiostanol, mepitiostane, testolactone; anti-adrenals such as aminoglutethimide, mitotane, trilostane; anti-androgens such as flutamide, nilutamide, bicalutamide, leuprolide, and goserelin; antibiotics such as aclacinomycins, actinomycin, anthramycin, azaserine, bleomycins, cactinomycin, calicheamicin, carubicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti estrogens including for example tamoxifen, raloxifene, aromatase inhibiting 4(5)-imidazoles, 4-hydroxytamoxifen, trioxifene, keoxifene, LY 117018, onapristone, and toremifene (Fareston); anti-metabolites such as methotrexate and 5-fluorouracil (5-FU); folic acid analogs such as denopterin, methotrexate, pteropterin, trimetrexate; aziridines such as benzodepa, carboquone, meturedepa, and uredepa; ethylenimines and methylmelamines including altretamine, triethylenemelamine, triethylenephosphoramide, triethylenethiophosphoramide and trimethylol melamine; folic acid replenisher such as folinic acid; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, ranimustine; platinum analogs such as cisplatin and carboplatin; vinblastine; platinum; proteins such as arginine deiminase and asparaginase; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; taxanes, e.g., paclitaxel (TAXOL®, Bristol-Myers Squibb Oncology, Princeton, N.J.) and docetaxel (TAXOTERE®), Rhone-Poulenc Rorer, Antony, France); topoisomerase inhibitor RFS 2000; thymidylate synthase inhibitor (such as Tomudex); additional chemotherapeutics including aceglatone; aldophosphamide glycoside; aminolevulinic acid; amsacrine; bestrabucil; bisantrene; edatrexate; defosfamide; demecolcine; diaziquone; difluoromethylornithine (DMFO); eflornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′, 2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; chlorambucil; gemcitabine; 6-thioguanine; mercaptopurine; methotrexate; etoposide (VP-16); ifosfamide; mitomycin C; mitoxantrone; vincristine; vinorelbine; Navelbine; Novantrone; teniposide; daunomycin; aminopterin; Xeloda; ibandronate; CPT-11; retinoic acid; esperamycins; capecitabine; and topoisomerase inhibitors such as irinotecan. Pharmaceutically acceptable salts, acids or derivatives of any of the above can also be used. In some examples, a modified anti-EGFR antibody provided herein is administered with irinotecan (see, e.g., Pfeiffer et al. (2007) Acta. Oncol. 46(5):697-701).

A chemotherapeutic agent can be administered as a prodrug. Examples of prodrugs that can be administered with an anti-EGFR antibody described herein include, but are not limited to, phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxy acetamide-containing prodrugs or optionally substituted phenylacetamide-containing prodrugs, 5-fluorocytosine and other 5-fluorouridine prodrugs which can be converted into the more active cytotoxic free drug.

In some examples, a modified anti-EGFR antibody provided herein is administered with one or more anti-angiogenic agents. For example, the anti-angiogenic factor can be a small molecule or a protein (e.g., an antibody, Fc fusion, or cytokine) that binds to a growth factor or growth factor receptor involved in promoting angiogenesis. Examples of anti-angiogenic agents include but are not limited to antibodies that bind to Vascular Endothelial Growth Factor (VEGF) or that bind to VEGF-R, RNA-based therapeutics that reduce levels of VEGF or VEGF-R expression, VEGF-toxin fusions, Regeneron's VEGF-trap, angiostatin (plasminogen fragment), antithrombin III, angiozyme, ABT-627, Bay 12-9566, BeneFin, bevacizumab, bisphosphonates, BMS-275291, cartilage-derived inhibitor (CDI), CAI, CD59 complement fragment, CEP-7055, Col 3, Combretastatin A-4, endostatin (collagen XVIII fragment), farnesyl transferase inhibitors, fibronectin fragment, GRO-beta, halofuginone, heparinases, heparin hexasaccharide fragment, HMV833, human chorionic gonadotropin (hCG), IM-862, interferon alpha, interferon beta, interferon gamma, interferon inducible protein 10 (IP-10), interleukin-12, kringle 5 (plasminogen fragment), marimastat, metalloproteinase inhibitors (e.g., TIMPs), 2-methoxyestradiol, MMI 270 (CGS 27023A), plasminogen activator inhibitor (PAI), platelet factor-4 (PF4), prinomastat, prolactin 16 kDa fragment, proliferin-related protein (PRP), PTK 787/ZK 222594, retinoids, solimastat, squalamine, SS3304, SU5416, SU6668, SU11248, tetrahydrocortisol-S, tetrathiomolybdate, thalidomide, thrombospondin-1 (TSP-1), TNP470, transforming growth factor beta (TGF-β) vasculostatin, vasostatin (calreticulin fragment), ZS6126, and ZD6474.

In some examples, a modified anti-EGFR antibody provided herein is administered with one or more tyrosine kinase inhibitors. Examples of tyrosine kinase inhibitors include but are not limited to quinazolines, such as PD 153035, 4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo(2,3-d) pyrimidines; curcumin (diferuloylmethane, 4,5-bis(4-fluoroanilino) phthalimide); tyrphostins containing nitrothiophene moieties; PD-0183805 (Warner-Lambert); antisense molecules (e.g., those that bind to ErbB-encoding nucleic acid); quinoxalines (U.S. Pat. No. 5,804,396); tyrphostins (U.S. Pat. No. 5,804,396); ZD6474 (Astra Zeneca); PTK-787 (Novartis/Schering A G); pan-ErbB inhibitors such as C1-1033 (Pfizer); Affinitac (ISIS 3521; Isis/Lilly); Imatinib mesylate (STI571, Gleevec®; Novartis); PKI 166 (Novartis); GW2016 (Glaxo SmithKline); C1-1033 (Pfizer); EKB-569 (Wyeth); Semaxinib (Sugen); ZD6474 (AstraZeneca); PTK-787 (Novartis/Schering A G); INC-1 C11 (ImClone); or as described in any of the following patent publications: U.S. Pat. No. 5,804,396; PCT WO 99/09016 (American Cyanamid); PCT WO 98/43960 (American Cyanamid); PCT WO 97/38983 (Warner-Lambert); PCT WO 99/06378 (Warner-Lambert); PCT WO 99/06396 (Warner-Lambert); PCT WO 96/30347 (Pfizer, Inc.); PCT WO 96/33978 (AstraZeneca); PCT WO 96/33979 (AstraZeneca); PCT WO 96/33980 (AstraZeneca), gefitinib (Iressa®, ZD1839, AstraZeneca), and OSI-774 (Tarceva®, OSI Pharmaceuticals/Genentech).

In some examples, a modified anti-EGFR antibody described herein is administered with one or more immunomodulatory agents. Such agents can increase or decrease production of one or more cytokines, up- or down-regulate self-antigen presentation, mask MHC antigens, or promote the proliferation, differentiation, migration, or activation state of one or more types of immune cells. Examples of immunomodulatory agents include but are not limited to non-steroidal anti-inflammatory drugs (NSAIDs) such as aspirin, ibuprofen, celecoxib, diclofenac, etodolac, fenoprofen, indomethacin, ketorolac, oxaprozin, nabumetone, sulindac, tolmetin, rofecoxib, naproxen, ketoprofen, and nabumetone; steroids (e.g., glucocorticoids, dexamethasone, cortisone, hydroxycortisone, methylprednisolone, prednisone, prednisolone, triamcinolone, azulfidine eicosanoids such as prostaglandins, thromboxanes, and leukotrienes; as well as topical steroids such as anthralin, calcipotriene, clobetasol, and tazarotene); cytokines such as TGFβ, IFNα, IFNβ, IFN-γ, IL-2, IL4, IL-10; cytokine, chemokine, or receptor antagonists including antibodies, soluble receptors, and receptor-Fc fusions against BAFF, B7, CCR2, CCR5, CD2, CD3, CD4, CD6, CD7, CD8, CD11, CD14, CD15, CD17, CD18, CD20, CD23, CD28, CD40, CD40L, CD44, CD45, CD52, CD64, CD80, CD86, CD147, CD152, complement factors (C5, D) CTLA4, eotaxin, Fas, ICAM, ICOS, IFNα, IFNβ, IFNγ, IFNAR, IgE, IL-1, IL-2, IL-2R, IL-4, IL-5R, IL-6, IL-8, IL-9 IL-12, IL-13, IL-13R1, IL-15, IL-18R, IL-23, integrins, LFA-1, LFA-3, MHC, selectins, TGFβ, TNFα, TNFβ, TNF-R1, T-cell receptor, including Enbrel® (etanercept), Humira® (adalimumab), and Remicade® (infliximab); heterologous anti-lymphocyte globulin; other immunomodulatory molecules such as 2-amino-6-aryl-5 substituted pyrimidines, anti-idiotypic antibodies for MHC binding peptides and MHC fragments, azathioprine, brequinar, Bromocryptine, cyclophosphamide, cyclosporine A, D-penicillamine, deoxyspergualin, FK506, glutaraldehyde, gold, hydroxychloroquine, leflunomide, malononitriloamides (e.g., leflunomide), methotrexate, minocycline, mizoribine, mycophenolate mofetil, rapamycin, and sulfasalazine.

In some examples, a modified anti-EGFR antibody described herein is administered with one or more cytokines. Examples of cytokines include but are not limited to lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL).

In some examples, a modified anti-EGFR antibody described herein is administered with one or more cytokines or other agents that stimulate cells of the immune system and enhance desired effector function. For example, agents that stimulate NK cells, including but not limited to IL-2 can be administered with an anti-EGFR antibody described herein. In another embodiment, agents that stimulate macrophages, including but not limited to C5a, formyl peptides such as N-formyl-methionyl-leucyl-phenylalanine (Beigier-Bompadre et. al. (2003) Scand. J. Immunol. 57: 221-8), can be administered with an anti-EGFR antibody described herein. Also, agents that stimulate neutrophils, including but not limited to G-CSF and GM-CSF, can be administered with an anti-EGFR antibody described herein. Furthermore, agents that promote migration of such immunostimulatory cytokines can be administered with an anti-EGFR antibody described herein. Also additional agents including, but not limited to, interferon gamma, IL-3 and IL-7 can promote one or more effector functions. In some examples, an anti-EGFR antibody described herein is administered with one or more cytokines or other agents that inhibit effector cell function.

In some examples, an anti-EGFR antibody described herein is administered with one or more antibiotics, including but not limited to: aminoglycoside antibiotics (e.g., apramycin, arbekacin, bambermycins, butirosin, dibekacin, gentamicin, kanamycin, neomycin, netilmicin, paromomycin, ribostamycin, sisomicin, spectinomycin), aminocyclitols (e.g., spectinomycin), amphenicol antibiotics (e.g., azidamfenicol, chloramphenicol, florfenicol, and thiamphenicol), ansamycin antibiotics (e.g., rifamide and rifampin), carbapenems (e.g., imipenem, meropenem, panipenem); cephalosporins (e.g., cefaclor, cefadroxil, cefamandole, cefatrizine, cefazedone, cefozopran, cefpimizole, cefpiramide, cefpirome, cefprozil, cefuroxime, cefixime, cephalexin, cephradine), cephamycins (cefbuperazone, cefoxitin, cefminox, cefmetazole, and cefotetan); lincosamides (e.g., clindamycin, lincomycin); macrolide (e.g., azithromycin, brefeldin A, clarithromycin, erythromycin, roxithromycin, tobramycin), monobactams (e.g., aztreonam, carumonam, and tigemonam); mupirocin; Oxacephems (e.g., flomoxef, latamoxef, and moxalactam); penicillins (e.g., amdinocillin, amdinocillin pivoxil, amoxicillin, bacampicillin, benzylpenicillinic acid, benzylpenicillin sodium, epicillin, fenbenicillin, floxacillin, penamecillin, penethamate hydriodide, penicillin o-benethamine, penicillin O, penicillin V, penicillin V benzoate, penicillin V hydrabamine, penimepicycline, and phenethicillin potassium); polypeptides (e.g., bacitracin, colistin, polymixin B, teicoplanin, vancomycin); quinolones (amifloxacin, cinoxacin, ciprofloxacin, enoxacin, enrofloxacin, fleroxacin, flumequine, gatifloxacin, gemifloxacin, grepafloxacin, lomefloxacin, moxifloxacin, nalidixic acid, norfloxacin, ofloxacin, oxolinic acid, pefloxacin, pipemidic acid, rosoxacin, rufloxacin, sparfloxacin, temafloxacin, tosufloxacin, and trovafloxacin); rifampin; streptogramins (e.g., quinupristin, dalfopristin); sulfonamides (sulfanilamide, sulfamethoxazole); tetracyclines (chlortetracycline, demeclocycline hydrochloride, demethylchlortetracycline, doxycycline, Duramycin, minocycline, neomycin, oxytetracycline, streptomycin, tetracycline, and vancomycin).

In some examples, a modified anti-EGFR antibody provided herein is administered with one or more anti-fungal agents, including but not limited to amphotericin B, ciclopirox, clotrimazole, econazole, fluconazole, flucytosine, itraconazole, ketoconazole, miconazole, nystatin, terbinafine, terconazole, and tioconazole. In some examples, an anti-EGFR antibody described herein is administered with one or more antiviral agents, including but not limited to protease inhibitors, reverse transcriptase inhibitors, and others, including type I interferons, viral fusion inhibitors, neuraminidase inhibitors, acyclovir, adefovir, amantadine, amprenavir, clevudine, enfuvirtide, entecavir, foscarnet, ganciclovir, idoxuridine, indinavir, lopinavir, pleconaril, ribavirin, rimantadine, ritonavir, saquinavir, trifluridine, vidarabine, and zidovudine.

A modified anti-EGFR antibody provided herein can be combined with other therapeutic regimens. For example, in one embodiment, the patient to be treated with a modified anti-EGFR antibody provided herein can receive radiation therapy. Radiation therapy can be administered according to protocols commonly employed in the art and known to the skilled artisan. Such therapy includes, but is not limited to, cesium, iridium, iodine, or cobalt radiation. The radiation therapy can be whole body irradiation, or can be directed locally to a specific site or tissue in or on the body, such as the lung, bladder, or prostate.

Typically, radiation therapy is administered in pulses over a period of time from about 1 to 2 weeks. The radiation therapy can, however, be administered over longer periods of time. For instance, radiation therapy can be administered to patients having head and neck cancer for about 6 to about 7 weeks. Optionally, the radiation therapy can be administered as a single dose or as multiple, sequential doses. The skilled medical practitioner can determine empirically the appropriate dose or doses of radiation therapy useful herein. In some examples, the anti-EGFR antibodies and optionally one or more other anti-cancer therapies are employed to treat cancer cells ex vivo. It is contemplated that such ex vivo treatment can be useful in bone marrow transplantation and particularly, autologous bone marrow transplantation. For instance, treatment of cells or tissue(s) containing cancer cells with an anti-EGFR antibody and one or more anti-cancer therapies, such as described herein, can be employed to deplete or substantially deplete the cancer cells prior to transplantation in a recipient patient.

Radiation therapy can also comprise treatment with an isotopically labeled molecule, such as an antibody. Examples of radioimmunotherapeutics include but Zevalin® (Y-90 labeled anti-CD20), LymphoCide® (Y-90 labeled anti-CD22) and Bexxar® (1-131 labeled anti-CD20).

In addition, it is contemplated that the modified anti-EGFR antibodies provided herein can be administered to a patient or subject in combination with still other therapeutic techniques such as surgery or phototherapy.

### H. EXAMPLES

The following examples are included for illustrative purposes only and are not intended to limit the scope of the invention.

### Example 1

### Generation and Expression of HC-Y104 Mutant Anti-EGFR Antibodies

Four (4) expression vectors encoding the light chain (LC) and modified heavy chain (HC) of cetuximab, separated by an internal ribosomal entry site (IRES), were generated in the pcDNA3.1-Erbitux-LC-IRES-HC backbone construct (SEQ ID NO: 306). The reference Cetuximab anti-EGFR antibody in the plasmid construct contains a sequence of nucleotides encoding an Igκ signal peptide (SEQ ID NO: 42) linked directly to the light chain sequence of nucleotides set forth in SEQ ID NO: 50 (encoding the light chain set forth in SEQ ID NO: 8). The reference Cetuximab anti-EGFR antibody in the plasmid construct also contains a sequence of nucleotides encoding an Ig signal peptide (SEQ ID NO: 41) linked directly to the heavy chain sequence of nucleotides set forth in SEQ ID NO: 48 (encoding the heavy chain set forth in SEQ ID NO: 6). The plasmid also encodes a FLAG tag (SEQ ID NO: 45) to be linked at the C-terminal end of the heavy chain constant domain.

Modified heavy chains were generated by mutating the coding sequences of the reference plasmid construct by codon substitutions to replace the nucleotides encoding Tyr (Y) at position 104 of the heavy chain amino acid sequence with those encoding Asp (D) or Glu (E). The Table 16 sets forth the mutant codons of the generated mutants, the expression vector encoding each modified anti-EGFR antibody, and the corresponding SEQ ID NO of the heavy and light chain of each generated variant.

TABLE 16
Sequences and mutant codons of generated
Y104D and Y104E mutants

| | | | Modified | | | | | | | |
|---|---|---|---|---|---|---|---|---|---|---|
| | | Expres- | Heavy Chain | | | | Light Chain | | | |
| | | sion | (SEQ ID NO) | | | | (SEQ ID NO) | | | |
| | Codon | vector | full- | | vari- | | full- | | vari- | |
| | substi- | (SEQ | length | | able | | length | | able | |
| | tution | ID NO) | nt | aa | nt | aa | nt | aa | nt | aa |
| HC-Y104D | GAU | 307 | 66 | 67 | 68 | 69 | 50 | 8 | 51 | 9 |
| HC-Y104D | GAC | 308 | | | | | | | | |
| HC-Y104E | GAA | 309 | 71 | 72 | 73 | 74 | 50 | 8 | 51 | 9 |
| HC-Y104E | GAG | 310 | | | | | | | | |

nt: nucleotide sequence
aa: amino acid sequence

FreeStyle CHO-S cells (Invitrogen) were grown to a density of 6×10⁵ cells/mL in 300 mL in a 1 L shaker flask and transfected with the above-generated constructs, using DNA FreeStyle MAX (Invitrogen) according to the manufacturer's instructions. The supernatants were harvested at 168 hr post transfection, and the expressed antibodies (mAbs) were purified using a 2-mL Protein A/G column (Bio-Rad). The eluted mAbs were dialyzed against phosphate buffered saline (PBS) and concentrated to a volume of 0.5-1 mL. The protein concentrations of the purified mAbs were determined using a NanoDrop spectrophotometer and the extinction coefficient, using the Beer-Lambert equation: A=εcl, where A is the absorbance, ε is the extinction coefficient, c is the protein concentration, and l is the path length. Table 17 sets forth the protein concentrations of the expressed antibodies.

TABLE 17
Protein Concentration

| | Codon | | | |
|---|---|---|---|---|
| | substi- | Conc. | Total | Total |
| | tution | mg/mL | Vol | Protein mg |
| HC-Y104D | GAU | 0.52 mg/mL | 0.9 mL | 0.47 mg |
| HC-Y104D | GAC | 0.87 mg/mL | 0.7 mL | 0.61 mg |
| HC-Y104D | GAA | 0.4 mg/mL | 0.5 mL | 0.2 mg |
| HC-Y104D | GAG | 0.37 mg/mL | 0.5 mL | 0.19 mg |

### Example 2

### Generation of Stable Cell Lines Expressing HC-Y104D Variant Anti-EGFR Antibody

To establish stable cell lines expressing HC-Y104D variant anti-EGFR antibody, 30 mL of CHO-S cells at an approximate density of 1.0×10⁶ cells/mL were transfected using 37.5 μg of plasmid DNA (SEQ ID NO: 308, generated in part 1 above) with 37.5 μL of FreeStyle™ MAX Reagent (Invitrogen) following the manufacturer's protocol.

Seventy two (72) hours post transfection, a 1-dimensional serial dilution strategy in CD-CHO media supplemented with GlutaMAX (8 mM) and 1 mg/mL G418 in 15 wells of 96-well round bottom plates (Nunc) was used for clonal isolation of cells. Four weeks later, clones expressing HC-Y104D mutants were screened by western blot analysis (WB) using peroxidase conjugated anti-human IgG Fc (Jackson Immunolab) as detecting antibody. Positive clones were expanded step-wise into 12-well, and then 6-well, plates, followed by T-25 and T-75 flasks and eventually into shaker flasks. Two clones, expressing at 5 mg/L of HC-Y104D, were further expanded to wavebag bioreactor production. The antibody were purified by affinity chromatography as described in Example 1, using a 30-mL Protein A column.

### Example 3

### Assessing pH-Dependent Binding of HC-Y104 Mutant Anti-EGFR Antibodies

The supernatants for flag-tagged Y104D-GAT, Y104D-GAC, Y104E-GAA, and Y104E-GAG, generated in Example 1, were assayed for binding to His-tagged soluble extracellular domain of EGFR (sEGFR-H6; Sino Biologics, Cat #10001-H08H) using a parallel, high-throughput pH sensitive ELISA under three pH conditions: pH 7.4, 6.5, and 6.0. Flag-tagged unmodified, wild-type Cetuximab anti-EGFR antibody (heavy chain set forth in SEQ ID NO: 18 and light chain set forth in SEQ ID NO: 8) and a flag-tagged humanized T030F/Y104D/Q111P mutant anti-EGFR mutant antibody (designated FDP-h3, see Example 15 in U.S. Publ. No. 2013/0266579) were used as control antibodies. FDP-h3 contains the sequence of nucleotides set forth in SEQ ID NO: 257 (light chain, encoding a light chain set forth in SEQ ID NO: 258) and the sequence of nucleotides set forth in SEQ ID NO: 64 (heavy chain, encoding a heavy chain set forth in SEQ ID NO: 65), where the heavy chain is linked directly at the C-terminus to a FLAG tag set forth in SEQ ID NO: 45.

Briefly, sEGFR-H6 was immobilized on 96 well Hi-bind plates (Costar #2592) by coating the plate overnight at 4° C. or for 2 hours at room temperature (RT) with 100 μL sEGFR-H6 antigen at 12 nM (1.32 μg/mL) in Buffer A Krebs-Ringer Buffer, pH 7.4, no serum (KRB, Sigma Aldrich, # K4002). The plates were then washed 3× with 250 μL/well of KRB. The plates were then divided into three groups and blocked, while covered, for 1 hr at RT with either 1) 250 μL pH 7.4 Buffer B (25% human serum and 1 mM lactic acid), 2) 250 μL pH 6.5 Buffer C (25% human serum and 16.7 mM lactic acid) or 3) 250 μL pH 6.0 Buffer C (25% human serum and 16.7 mM lactic acid).

The flag-tagged Y104D-GAT, Y104D-GAC, Y104E-GAA, and Y104E-GAG antibodies and the control wild-type and FDP-h3 antibodies were diluted by three-fold serial dilutions to generate seven working concentrations of each antibody under each of the pH conditions. For testing binding at pH 7.4, the Y104D-GAT, Y104D-GAC, Y104E-GAA, and Y104E-GAG mutants and control FDP-h3) were each diluted to 1000 ng/mL, 333 ng/mL, 111 ng/mL, 37 ng/mL, 12.3 ng/mL, 4.1 ng/mL, and 1.4 ng/mL in Buffer B, pH 7.4. For testing at pH 6.5 and 6.0, the Y104D-GAT, Y104D-GAC, Y104E-GAA, and Y104E-GAG mutants and control FDP-h3 were each diluted to 300 ng/mL, 100 ng/mL, 33.3 ng/mL, 11.1 ng/mL, 3.7 ng/mL, 1.2 ng/mL, and 0.4 ng/mL in Buffer C, pH 6.5 or 6.0, respectively. Wild-type Cetuximab was diluted to 100 ng/mL, 33.3 ng/mL, 11.1 ng/ml, 3.7 ng/ml, 1.2 ng/ml, 0.4 ng/mL, and 0.14 ng/mL in each of the 3 buffers at pH 7.4, 6.5 and 6.0. One hundred microliters (100 μL) of each of the antibody dilutions were added to separate wells of the 96-well plates containing the bound sEGFR-H6 antigen, which were covered and incubated at RT for 1 hr.

After incubation, the plate was washed 3× with 250 μL/well of Buffer B or Buffer C at the corresponding pH. 100 μL goat anti-FLAG-HRP detection antibody (Abcam, #ab 1238) at 500 ng/mL in Buffer B or Buffer C at the corresponding pH were added to each well. The plates were then covered and incubated for 1 hr at RT. The wells of the plates were then washed 3× with 250 μL of Buffer B or Buffer C at the corresponding pH. Finally, 100 μL SureBlue TMB Microwell Peroxidase Substrate 1-component (KPL, #52-00-03) solution was added to each well, and the plate was allowed to develop for 15-20 minutes at RT (away from light). The reaction was stopped by adding 100 μL TMB stop solution (KPL, #50-85-06) to each well, and the optical density of the wells was measured at 450 nM (OD₄₅₀) within 30 min using a Microplate Spectrophotometer (Molecular Devices, Spectra Max M3).

The ELISA was performed in triplicate, and the average OD values of the reactions were calculated for each sample and plotted with respect to the antibody concentration. The 4 Parameter Logistic nonlinear regression model was used for curve-fitting analysis of the results using the following equation: y=((A−D)/(1+((x/C)̂B)))+D, where A is the minimum asymptote, B is the slope factor, C is the inflection point/EC₅₀ value, and D is the maximum asymptote. The results are set forth in Tables 18-20 below.

TABLE 18
Binding at pH 7.4

| | A | B | C | D | |
|---|---|---|---|---|---|
| | (minimum) | (slope) | (EC₅₀) | (maximum) | R\hat{ }2 |
| Wild-type | 0.238 | 1.56 | 3.3 | 3.93 | 0.998 |
| Y104D-GAT | 0.153 | 1.24 | 6.18 | 3.74 | 1 |
| Y104D-GAC | 0.161 | 1.29 | 5.07 | 3.79 | 0.999 |
| Y104E-GAA | 0.131 | 0.956 | 18.6 | 3.81 | 1 |
| Y104E-GAG | 0.141 | 1.13 | 16.9 | 3.78 | 0.999 |
| FDP-h3 | 0.174 | 1.09 | 32.6 | 3.65 | 0.998 |

TABLE 19
Binding at pH 6.5

| | A | B | C | D | |
|---|---|---|---|---|---|
| | (minimum) | (slope) | (EC₅₀) | (maximum) | R\hat{ }2 |
| Wild-type | 0.234 | 1.49 | 2.28 | 3.88 | 0.999 |
| Y104D-GAT | 0.202 | 1.41 | 2.89 | 3.8 | 0.998 |
| Y104D-GAC | 0.194 | 1.46 | 2.36 | 3.79 | 0.999 |
| Y104E-GAA | 0.181 | 1.45 | 2.57 | 3.79 | 0.999 |
| Y104E-GAG | 0.162 | 1.36 | 2.65 | 3.82 | 0.999 |
| FDP-h3 | 0.22 | 1.42 | 4.87 | 3.76 | 1 |

TABLE 20
Binding at pH 6.0

| | A | B | C | D | |
|---|---|---|---|---|---|
| | (minimum) | (slope) | (EC₅₀) | (maximum) | R\hat{ }2 |
| Wild-type | 0.195 | 1.42 | 2.6 | 3.91 | 0.999 |
| Y104D-GAT | 0.164 | 1.41 | 3.4 | 3.81 | 0.999 |
| Y104D-GAC | 0.175 | 1.42 | 3.1 | 3.84 | 0.999 |
| Y104E-GAA | 0.169 | 1.39 | 2.88 | 3.86 | 0.998 |
| Y104E-GAG | 0.162 | 1.4 | 2.89 | 3.87 | 0.999 |
| FDP-h3 | 0.175 | 1.39 | 4.33 | 3.79 | 0.999 |

The EC₅₀ values at the different pH conditions for each tested mutant and controls are further summarized in Table 21, where a higher EC₅₀ indicates weaker binding. The Table also sets forth the ratio of binding activity of each mutant at pH 6.0 or 6.5 versus 7.4 (i.e., quotient of the inverse of the EC₅₀ at pH 6.0 or 6.5 versus pH 7.4), where a ratio >1 indicates binding is greater under the acidic pH condition than the neutral pH condition.

TABLE 21
EGFR Binding at pH 6.0, 6.5 and 7.4

| | pH 6.0 | pH 6.5 | pH 7.4 | 6.0/7.4 | 6.5/7.4 |
|---|---|---|---|---|---|
| Wild-type | 2.6 | 2.28 | 3.3 | 1.27 | 1.45 |
| Y104D-GAT | 3.4 | 2.89 | 6.18 | 1.82 | 2.14 |
| Y104D-GAC | 3.1 | 2.36 | 5.07 | 1.64 | 2.15 |
| Y104E-GAA | 2.88 | 2.57 | 18.6 | 6.46 | 7.24 |
| Y104E-GAG | 2.89 | 2.65 | 16.9 | 5.85 | 6.38 |
| FDP-h3 | 4.33 | 4.87 | 32.6 | 7.53 | 6.69 |

The results show that at pH 7.4, the wild-type cetuximab antibody exhibited a slightly higher EC₅₀ than at pH 6.5 or pH 6.0. In contrast, for the Y104 mutants and the FDP-h3 control, binding was substantially weaker at pH 7.4 than at pH 6.5 or pH 6.0 as evidenced by a higher EC₅₀ under the neutral pH tested conditions than the acidic pH tested conditions. Thus, each of the mutants exhibit a greater ratio of binding at acidic pH 6.0 or 6.5 than at pH 7.4.

At pH 7.4, the Y104E mutant exhibited an EC₅₀ value that was about 3-fold greater than the EC₅₀ value of the Y104D mutant, showing that the Y104E mutant exhibits weaker binding at pH 7.4 than the Y104E mutant. At acidic pH conditions of 6.0 and 6.5, the binding of the Y104E and Y104D mutants to EGFR was substantially the same as demonstrated by similar EC₅₀ values. Specifically, the Y104E mutants, Y104E-GAA and Y104E-GAG, exhibited EC₅₀ values of 18.6 and 16.9 at pH 7.4, which was more than 5-fold higher than that of the wild-type antibody at neutral pH (EC₅₀=3.3) and approximately 6-fold higher than the corresponding EC₅₀ values at the more acidic pHs. These results indicate the substitution of Tyr (Y) with Glu (E) at position 104 results in an antibody with reduced EGFR binding at neutral pH compared to wild-type and compared to antibodies with Asp (D) at position 104, but that retain similar levels of EGFR binding under acidic conditions.

### Example 4

### Generation of Humanized Y104D, Y104E and Y104E/Q111P Antibodies

1. Humanization and Screening for pH-Dependence

Double stranded DNA fragments encoding the full-length light chain and heavy chain sequences of HC-Y104D/Q111P (clone 2-2; also called DP; see U.S. patent application Ser. No. 13/815,553) with a heavy chain set forth in SEQ ID NO: 53 (encoded by a nucleic acid sequence set forth in SEQ ID NO: 52) and a light chain set forth in SEQ ID NO: 8 (encoded by a nucleic acid sequence set forth in SEQ ID NO: 50) was used to generate a library of humanized clones that were then expressed and screened for pH-dependent EGFR binding and protein expression levels (see Example 15 in U.S. Publ. No. 2013/0266579). CHO-S cells were plated in 96-well plates and transfected with the humanized clones. The supernatants were collected 48 hours post transfection. The IgG concentration was determined and supernatants were adjusted to 2 ng/mL and were tested for pH-dependent binding of EGFR binding at pH 6.0 and pH 7.4 using the pH sensitive ELISA described in Example 3.

Primary hits were selected that exhibited similar or better ratios of binding activity at pH 6.0 versus binding activity at pH 7.4 compared to the parental positive control (HC-Y104D/Q111P), excluding clones with low expression levels. The primary hits were subjected to a secondary construction and confirmation screening. For screening, transfected supernatant was adjusted to concentrations of 4 ng/mL, 2 ng/mL and 1 ng/mL and were tested for pH-dependent binding of EGFR binding at pH 6.0 and pH 7.4 using the pH sensitive ELISA described in Example 3. Hits that exhibited similar or better ratios of binding activity at pH 6.0 versus binding activity at pH 7.4 compared to the parental positive control were identified, and the sequences of the identified hits were determined

2. Generation of Variants from Humanized Backbone

The humanized Y104D/Q111P cetuximab mutant antibody (designated DP-h7; SEQ ID NOS: 55 (heavy chain) and 181 (light chain)) was generated and selected as a humanized hit as described above. In the confirmation screen, the DP-h7 humanized clone exhibited a pH 6.0/pH 7.4 OD ratio at the tested concentrations as follows: 4 ng/mL, ratio of 11.30; 2 ng/mL, ratio of 7.21; 1 ng/mL, ratio of 17.35.

The plasmid encoding DP-h7 set forth in SEQ ID NO: 311, containing the nucleotide sequences set forth in SEQ ID NO: 54 (heavy chain) and 180 (light chain)), was used as a starting backbone to generate humanized Y104D, Y104E and Y104E/Q111P mutant EGFR antibodies. Briefly, the humanized backbone was used to mutate the Asp (D) at position 104 to Glu (E) and/or the Pro (P) at position 111 to Gln (Q). Table 22 summarizes the mutated codons, the generated expression vectors and the resulting nucleotide and amino acid sequences of the generated humanized antibodies.

TABLE 22
Humanized Clones

| | position | position | Expression | Modified Heavy Chain | | | | Modified Light Chain | | | |
|---|---|---|---|---|---|---|---|---|---|---|---|
| | 104 | 111 | Vector | (SEQ ID NO) | | | | (SEQ ID NO) | | | |
| | (codon: | (codon: | (SEQ ID | full-length | | variable | | full-length | | variable | |
| | 310-312) | 331-333) | NO) | nt | aa | nt | aa | nt | aa | nt | aa |
| DP-h7 | Y104D | Q111P | 311 | 54 | 55 | 54 (nt | 55 (aa | 180 | 181 | 182 | 183 |
| (back- | (GAT) | (CCT) | | | | 1-357) | 1-119) | | | | |
| bone) | | | | | | | | | | | |
| D-h | Y104D | Q111 | 312 | 56 | 57 | 56 (nt | 57 (aa | 180 | 181 | 182 | 183 |
| | (GAT) | (CAG) | | | | 1-357) | 1-119) | | | | |
| E-h | Y104E | Q111 | 313 | 58 | 59 | 60 | 61 | 180 | 181 | 182 | 183 |
| | (GAG) | (CAG) | | | | | | | | | |
| EP-h | Y104E | Q111P | 314 | 134 | 135 | 136 | 137 | 180 | 181 | 182 | 183 |
| | (GAG) | (CCT) | | | | | | | | | |

### 3. Assessing Expression of Humanized Y104D, Y104E and Y104E/Q111P Antibodies

Plasmids encoding the humanized Y104D (D-h), Y104E (E-h) and Y104E/Q111P

(EP-h) clones were expressed in FreeStyle CHO-S cells, purified and concentrated as described in Example 1, except the supernatants were harvested 96 hr post transfection and were concentrated to a final volume of 4.5 mL. The protein concentrations of the expressed antibodies were determined, using a NanoDrop spectrophotometer and the extinction coefficient, as described in Example 1. Table 23 sets forth the protein concentrations of the expressed antibodies.

TABLE 23
Protein Concentration

| | Codon | | | |
|---|---|---|---|---|
| | substi- | Conc. | Total | |
| Construct | tution | mg/mL | Vol | Total Protein mg |
| DP-h7 | GAT | 0.13 mg/mL | 4.5 mL | 0.59 mg (2 mg/L) |
| (back-bone) | | | | |
| D-h | GAT | 0.34 mg/mL | 4.5 mL | 1.53 mg (2.6 mg/L) |
| E-h | GAG | 0.31 mg/mL | 4.5 mL | 1.4 mg (2.3 mg/L) |
| EP-h | GAG | 0.24 mg/mL | 4.5 mL | 1.1 mg (1.8 mg/L) |

4. Assessing pH-Dependent Activity of Humanized Y104D (D-h), Y104E (E-h) and Y104E/Q111P (EP-h) Antibodies

a.

The humanized Y104D (D-h), Y104E (E-h) and Y104E/Q111P (EP-h) were purified as described in subsection 3 and assayed for binding to His-tagged soluble extracellular domain of EGFR (sEGFR-H6; Sino Biologics, Cat #10001-H08H) using a parallel, high-throughput pH sensitive ELISA under three pH conditions: pH 7.4, 6.5, and 6.0 as described in Example 3. The binding activities of non-humanized flag-tagged Y104D (heavy chain set forth in SEQ ID NO: 67 and light chain set forth in SEQ ID NO: 8) and non-humanized flag-tagged Y104D/Q111P (heavy chain set forth in SEQ ID NO: 53 and light chain set forth in SEQ ID NO: 8) were used as reference/control antibodies.

The ELISA was performed in triplicate, and the average OD values of the reactions were calculated for each sample and plotted with respect to the antibody concentration using the 4 Parameter Logistic nonlinear regression model described in Example 3. The results are set forth in Tables 24-26 below.

TABLE 24
Binding at pH 7.4

| | A | B | C | D | |
|---|---|---|---|---|---|
| | (minimum) | (slope) | (EC₅₀) | (maximum) | R\hat{ }2 |
| Y104D | 0.225 | 1.2 | 8.33 | 3.78 | 0.998 |
| Y104D/Q111P | 0.173 | 1.05 | 18.9 | 3.43 | 1 |
| DP-h07 | 0.192 | 1.13 | 33.6 | 3.14 | 0.997 |
| D-h | 0.179 | 1.06 | 30.2 | 3.45 | 0.999 |
| E-h | 0.195 | 1.09 | 46.1 | 3.23 | 0.995 |
| EP-h | 0.227 | 1.19 | 69.8 | 2.91 | 0.998 |

TABLE 25
Binding at pH 6.5

| | A | B | C | D | |
|---|---|---|---|---|---|
| | (minimum) | (slope) | (EC₅₀) | (maximum) | R\hat{ }2 |
| Y104D | 0.243 | 1.49 | 4.09 | 3.87 | 0.998 |
| Y104D/Q111P | 0.225 | 1.43 | 8.96 | 3.85 | 0.998 |
| DP-h07 | 0.222 | 1.41 | 6.91 | 3.83 | 0.999 |
| D-h | 0.222 | 1.46 | 7.57 | 3.84 | 0.999 |
| E-h | 0.205 | 1.51 | 5.87 | 3.85 | 0.999 |
| EP-h | 0.201 | 1.45 | 6.37 | 3.89 | 0.999 |

TABLE 26
Binding at pH 6.0

| | A | B | C | D | |
|---|---|---|---|---|---|
| | (minimum) | (slope) | (EC₅₀) | (maximum) | R\hat{ }2 |
| Y104D | 0.245 | 1.5 | 3.91 | 3.88 | 0.998 |
| Y104D/Q111P | 0.29 | 1.46 | 7.19 | 3.9 | 0.996 |
| DP-h07 | 0.297 | 1.67 | 4.15 | 3.83 | 0.998 |
| D-h | 0.266 | 1.58 | 4.87 | 3.89 | 0.998 |
| E-h | 0.222 | 1.45 | 3.18 | 3.92 | 0.997 |
| EP-h | 0.281 | 1.59 | 3.92 | 3.92 | 0.997 |

The EC₅₀ values at the different pH conditions for each tested mutant and controls are further summarized in Table 27, where a higher EC₅₀ indicates weaker binding. The Table also sets forth the ratio of binding activity of each mutant at pH 6.0 or 6.5 versus 7.4 (i.e., quotient of the inverse of the EC₅₀ at pH 6.0 or 6.5 versus pH 7.4), where a ratio >1 indicates binding is greater under the acidic pH condition than the neutral pH condition.

TABLE 27
EGFR Binding at pH 6.0, 6.5 and 7.4

| | pH 6.0 | pH 6.5 | pH 7.4 | 6.0/7.4 | 6.5/7.4 |
|---|---|---|---|---|---|
| Y104D | 3.91 | 4.09 | 8.33 | 2.13 | 2.04 |
| Y104D/Q111P | 7.19 | 8.96 | 18.9 | 2.62 | 2.11 |
| DP-h07 | 4.15 | 6.91 | 33.6 | 8.10 | 4.86 |
| D-h | 4.87 | 7.57 | 30.2 | 6.20 | 3.99 |
| E-h | 3.18 | 5.87 | 46.1 | 14.5 | 7.85 |
| EP-h | 3.92 | 6.37 | 69.8 | 17.81 | 10.96 |

The results show that all tested variants exhibit a higher EC₅₀, and hence weaker binding, at pH 7.4 than at pH 6.5 or pH 6.0. The binding activity as evidenced by the EC₅₀ of the tested mutants were all substantially the same at pH 6.0 and pH 6.5, although the variant Y104D/Q111P exhibited slightly decreased binding activity at pH 6.0 and 6.5 compared to the other tested variants. Specifically, the EC₅₀ values for non-humanized Y104D, DP-h07, D-h, E-h, and EP-h antibodies were similar at pH 6.0, ranging in values from approximately 3 to 4. The EC₅₀ of non-humanized Y104D/Q111P was slightly higher at pH 6.0 (EC₅₀=7.2). The mutants also exhibited similar EC₅₀ values at pH 6.5. Thus, at acidic pH, all the constructs tested have similar EGFR-binding affinity.

Each of the mutants exhibited a greater ratio of binding activity at acidic pH 6.0 or 6.5 than at pH 7.4. The humanized Y104E (E-h) and Y104D/Q111P (EP-h) exhibited the highest ratio of binding activity at acidic pH 6.0 or 6.5 than at pH 7.4 of the constructs tested. The E-h and EP-h mutants variants also exhibited the highest EC₅₀ values, and hence weakest binding activity, at pH 7.4, which were 46.1 and 69.8 at pH 7.4, respectively. Thus, the higher ratio of binding activity, as measured by the ratio of the inverse of the EC₅₀ at pH 6.0 or 6.5 versus pH 7.4, are due to reduced binding affinities (increased EC₅₀ values) at neutral pH.

b.

In a further experiment, the purified, humanized Y104D (D-h), Y104E (E-h) and Y104E/Q111P (EP-h) were assayed for binding to His-tagged soluble extracellular domain of EGFR (sEGFR-H6; Sino Biologics, Cat #10001-H08H) using a parallel, high-throughput pH sensitive ELISA under three pH conditions: pH 7.4, 6.5, and 6.0 as described above. For comparison, the binding activities of non-humanized flag-tagged Y104D (heavy chain set forth in SEQ ID NO: 67 and light chain set forth in SEQ ID NO: 8), non-humanized flag-tagged Y104E (heavy chain set forth in SEQ ID NO: 71 and light chain set forth in SEQ ID NO: 8), and non-humanized flag-tagged Y104D, which was purified from an established stable CHO cell line that expresses the heavy chain set forth in SEQ ID NO: 67 and light chain set forth in SEQ ID NO: 8, designated Y104D-S.

The ELISA was performed in triplicate, and the average OD values of the reactions were calculated for each sample and plotted with respect to the antibody concentration using the 4 Parameter Logistic nonlinear regression model as described above. The results are set forth in Tables 28-30 below.

TABLE 28
Binding at pH 7.4

| | A | B | C | D | |
|---|---|---|---|---|---|
| | (minimum) | (slope) | (EC₅₀) | (maximum) | R\hat{ }2 |
| Y104D-S | 0.253 | 1.04 | 16 | 3.79 | 0.998 |
| Y104D | 0.174 | 1.04 | 10.6 | 3.7 | 0.999 |
| Y104E | 0.229 | 0.927 | 38.9 | 2.89 | 0.997 |
| D-h | 0.235 | 0.944 | 56.4 | 2.03 | 0.990 |
| E-h | 0.251 | 1.76 | 59.3 | 1.44 | 0.996 |
| EP-h | 0.198 | 0.825 | 209 | 1.83 | 0.999 |

TABLE 29
Binding at pH 6.5

| | A | B | C | D | |
|---|---|---|---|---|---|
| | (minimum) | (slope) | (EC₅₀) | (maximum) | R\hat{ }2 |
| Y104D-S | 0.176 | 1.41 | 5.07 | 3.86 | 0.999 |
| Y104D | 0.232 | 1.54 | 4.58 | 3.86 | 0.998 |
| Y104E | 0.215 | 1.42 | 4.31 | 3.81 | 0.999 |
| D-h | 0.213 | 1.41 | 12.7 | 3.82 | 0.999 |
| E-h | 0.185 | 1.3 | 9.82 | 3.94 | 0.998 |
| EP-h | 0.197 | 1.23 | 12.1 | 3.93 | 0.997 |

TABLE 30
Binding at pH 6.0

| | A | B | C | D | |
|---|---|---|---|---|---|
| | (minimum) | (slope) | (EC₅₀) | (maximum) | R\hat{ }2 |
| Y104D-S | 0.223 | 1.61 | 4.27 | 3.84 | 0.999 |
| Y104D | 0.241 | 1.55 | 3.96 | 3.82 | 0.997 |
| Y104E | 0.243 | 1.58 | 3.21 | 3.83 | 0.996 |
| D-h | 0.189 | 1.45 | 6.77 | 3.88 | 0.999 |
| E-h | 0.228 | 1.67 | 4.82 | 3.87 | 0.998 |
| EP-h | 0.21 | 1.54 | 4.28 | 3.91 | 0.998 |

The EC₅₀ values at the different pH conditions for each tested mutant and controls are further summarized in Table 31, and the ratio of binding activity of each mutant at pH 6.0 or 6.5 versus 7.4 (i.e., quotient of the inverse of the EC₅₀ at pH 6.0 or 6.5 versus pH 7.4) is provided.

TABLE 31
EGFR Binding at pH 6.0, 6.5 and 7.4

| | pH 6.0 | pH 6.5 | pH 7.4 | 6.0/7.4 | 6.5/7.4 |
|---|---|---|---|---|---|
| Y104D-S | 4.27 | 5.07 | 16 | 3.75 | 3.16 |
| Y104D | 3.96 | 4.58 | 10.6 | 2.68 | 2.31 |
| Y104E | 3.21 | 4.31 | 38.9 | 12.12 | 9.03 |
| D-h | 6.77 | 12.7 | 56.4 | 8.33 | 4.44 |
| E-h | 4.82 | 9.82 | 59.3 | 12.30 | 6.04 |
| EP-h | 4.28 | 12.1 | 209 | 48.83 | 17.3 |

The results indicate the Y104E mutants exhibited greater pH selective binding at acidic pH than the Y104D mutants. For example, the non-humanized Y104E mutant exhibited reduced binding at pH 7.4, with an EC₅₀ at pH 7.4 of 38.9, than at pH 6.0 (EC₅₀=3.21) or at pH 6.5 (EC₅₀=4.31). The humanized Y104E (E-h) antibody exhibited slightly decreased binding activity at pH 7.4 than the corresponding non-humanized Y104E antibody, although the ratio of binding activity at pH 6.0 or 6.5 versus 7.4 was substantially the same as those for non-humanized Y104E antibody. The humanized Y104E/Q111P mutant (EP-h) exhibited the highest selectivity for EGFR binding under acidic conditions of the constructs tested. Specifically, the EP-h mutant exhibited an EC₅₀ 209 at pH 7.4, which was approximately 49-fold higher than the corresponding EC₅₀ value pH 6.0 and approximately 17-fold higher than the corresponding EC₅₀ value pH 6.5, showing that the EP-h humanized variant exhibits substantially weaker binding at pH 7.4 than the other variants tested. The binding activity of the EP-h variant at acidic pH 6.0 or 6.5 as demonstrated by the EC₅₀ values was similar to the other tested variants.

The Y104D mutants also exhibited acidic-pH selective binding activity, but to a lesser extent than was demonstrated by the Y104E mutants. The humanized Y104D (D-h) mutant exhibited an EC₅₀ at pH 7.4 of 56.4, which was approximately 8-fold higher than the E₅₀ at pH 6.0 and approximately 4-fold higher than the EC₅₀ at pH 6.5.

### Example 5

### Generation of and Screening for Anti-EGFR Mutants with pH-Dependent EGFR Binding

1. Generating a Library of Anti-EGFR Mutant Antibodies

A library of single point mutants of the Cetuximab anti-EGFR antibody was constructed and generated by site-directed mutagenesis in the pcDNA3.1-Erbitux-LC-IRES-HC backbone construct (SEQ ID NO: 306). The construct contains a reference cetuximab anti-EGFR antibody plasmid construct that contains a sequence of nucleotides encoding an Igκ signal peptide (SEQ ID NO: 42) linked directly to the light chain sequence of nucleotides set forth in SEQ ID NO: 50 (encoding the light chain set forth in SEQ ID NO: 8). The reference Cetuximab anti-EGFR antibody in the plasmid construct also contains a sequence of nucleotides encoding an Ig signal peptide (SEQ ID NO: 41) linked directly to the heavy chain sequence of nucleotides set forth in SEQ ID NO: 48 (encoding the heavy chain set forth in SEQ ID NO: 6). The plasmid also contained a FLAG tag (SEQ ID NO: 45) linked at the C-terminal end of the heavy chain constant domain.

The library was generated to contain variants of Cetuximab anti-EGFR antibody, whereby each member contained a single amino acid mutation compared to the reference antibody at one of one hundred amino acid positions within the variable regions of either the heavy chain (SEQ ID NO: 6 with the variable heavy chain set forth in SEQ ID NO: 7) or light chain (SEQ ID NO: 8 with the variable light chain set forth in SEQ ID NO: 9) of Cetuximab. The positions that were varied were in the variable region of the light and heavy chains of the Cetuximab anti-EGFR antibody, with the majority of positions in the CDRs of the light or heavy chain. At least 15 amino acid mutations were made at each position. Each member of the library was sequenced, and glycerol stocks of members of the library were prepared and stored at −80° C.

2. Screening Anti-EGFR Mutants

Plasmid DNA was transfected into monolayer CHO-S cells (Invitrogen, Cat. No. 11619-012) using Lipofectamine 2000 (Invitrogen, Cat. No. 11668-027) following the manufacturer's protocol. Briefly, CHO-S cells were seeded the night before transfection and grown in DMEM with 10% Fetal Bovine Serum (FBS). The next day, after the cells were 80% confluent, the medium of the CHO-S cells was replaced with Opti-MEM (Invitrogen). A mixture of plasmid DNA and Lipofectamine (0.2 μg DNA and 0.5 μL Lipofetamine) was added to the CHO-S cells and incubated overnight. The next day, the cells were supplemented with CD-CHO serum free media (Invitrogen, Cat. No. 10743-029). Supernatant from transfected cells was collected after transfection (generally 72 hours after transfection).

The supernatants were assayed for binding to soluble extracellular domain of EGF receptor (EGFR sECD) using a parallel, high-throughput pH sensitive ELISA, as described in Example 3, except the antibodies were added at two dilutions (Dilution 1 and Dilution 2) and binding was measured under two pH conditions: pH 7.4 and pH 6.0.

a. Antibody Binding Results

The ELISA was performed in duplicate, and the average OD values of the duplicate reactions were calculated. Based on the OD value, variant anti-EGFR antibodies that exhibited higher binding activity to sEGFR-H6 at pH 6.0 compared to at pH 7.0 were identified and are set forth in Table 32. The Table sets forth the average OD at pH 6.0 (OD_{pH 6.0}), average OD at pH 7.4 (OD_{pH 7.4}), and the ratio of the average OD values at pH 6.0 and 7.4 (OD_{pH 6.0}/OD_{pH 7.4}) for the variant antibodies at Dilution 1 and Dilution 2.

TABLE 32
Variant anti-EGFR antibodies

| | | | | | | OD_{pH 6.0}/ | |
|---|---|---|---|---|---|---|---|
| | | Average OD_{pH 6.0} | | Average OD_{pH 67.4} | | OD_{pH 7.4} | |
| | Muta- | Dilu- | Dilu- | Dilu- | Dilu- | Dilu- | Dilu- |
| Chain | tion | tion 1 | tion 2 | tion 1 | tion 2 | tion 1 | tion 2 |
| HC | T23K | 2.6495 | 1.048 | 2.125 | 0.619 | 1.25 | 1.695 |
| HC | T23H | 2.744 | 1.5525 | 2.3405 | 0.833 | 1.173 | 1.851 |
| HC | T23R | 2.5055 | 1.2625 | 2.061 | 0.6245 | 1.216 | 2.03 |
| HC | T23A | 2.8735 | 1.142 | 2.5135 | 0.5 | 1.15 | 2.283 |
| HC | T23C | 2.654 | 1.3115 | 2.2505 | 0.687 | 1.179 | 1.909 |
| HC | T23E | 2.8785 | 1.3525 | 2.678 | 0.667 | 1.075 | 2.028 |
| HC | T23G | 1.679 | 0.3445 | 0.9585 | 0.1655 | 1.753 | 2.08 |
| HC | T23I | 2.709 | 1.4085 | 2.309 | 0.81 | 1.175 | 1.736 |
| HC | T23M | 2.3595 | 0.8185 | 1.772 | 0.504 | 1.332 | 1.636 |
| HC | T23N | 2.627 | 1.0915 | 1.823 | 0.6175 | 1.45 | 1.778 |
| HC | T23P | 0.252 | 0.1 | 0.1395 | 0.0965 | 1.812 | 1.035 |
| HC | T23S | 1.644 | 1.2745 | 1.9785 | 0.692 | 0.832 | 1.841 |
| HC | T23V | 0.258 | 0.1445 | 0.1775 | 0.106 | 1.454 | 1.365 |
| HC | T23W | 2.346 | 0.8765 | 1.8475 | 0.3025 | 1.274 | 2.896 |
| HC | T23L | 2.602 | 0.576 | 1.7855 | 0.2815 | 1.575 | 2.048 |
| HC | V24R | 0.091 | 0.085 | 0.079 | 0.071 | 1.158 | 1.194 |
| HC | V24A | 3.065 | 1.568 | 2.184 | 0.523 | 1.403 | 3.003 |
| HC | V24E | 0.780 | 0.232 | 0.300 | 0.114 | 2.596 | 2.044 |
| HC | V24F | 2.386 | 0.645 | 1.156 | 0.336 | 2.057 | 2.937 |
| HC | V24G | 3.144 | 1.932 | 2.687 | 0.716 | 1.170 | 2.701 |
| HC | V24I | 1.669 | 0.485 | 0.590 | 0.176 | 2.837 | 2.761 |
| HC | V24M | 2.765 | 0.957 | 1.311 | 0.350 | 2.110 | 2.738 |
| HC | V24P | 1.512 | 0.388 | 0.511 | 0.165 | 2.961 | 2.355 |
| HC | V24S | 3.093 | 1.588 | 2.109 | 0.533 | 1.467 | 2.979 |
| HC | V24T | 2.605 | 0.821 | 1.091 | 0.276 | 2.389 | 2.983 |
| HC | V24L | 1.678 | 0.538 | 0.431 | 0.146 | 3.889 | 3.695 |
| HC | S25H | 3.006 | 1.752 | 1.255 | 0.311 | 2.456 | 5.667 |
| HC | S25R | 3.104 | 1.367 | 1.807 | 0.388 | 1.721 | 3.484 |
| HC | S25A | 3.206 | 2.225 | 2.164 | 0.563 | 1.481 | 3.957 |
| HC | S25C | 2.947 | 1.369 | 1.858 | 0.431 | 1.586 | 3.184 |
| HC | S25D | 3.076 | 1.717 | 2.194 | 0.578 | 1.487 | 3.073 |
| HC | S25E | 3.099 | 1.210 | 2.658 | 0.663 | 1.166 | 1.827 |
| HC | S25F | 3.135 | 1.758 | 2.822 | 0.787 | 1.111 | 2.234 |
| HC | S25G | 2.937 | 1.218 | 1.142 | 0.317 | 2.579 | 3.845 |
| HC | S25I | 3.042 | 2.171 | 1.994 | 0.494 | 1.525 | 4.394 |
| HC | S25M | 3.158 | 2.444 | 2.774 | 0.759 | 1.138 | 3.230 |
| HC | S25P | 0.899 | 0.240 | 0.250 | 0.107 | 3.629 | 2.240 |
| HC | S25Q | 1.999 | 0.527 | 0.495 | 0.146 | 4.034 | 3.628 |
| HC | S25T | 2.795 | 0.510 | 1.483 | 0.162 | 1.886 | 1.567 |
| HC | S25V | 3.245 | 2.478 | 2.331 | 0.804 | 1.393 | 3.082 |
| HC | S25L | 3.155 | 1.773 | 1.631 | 0.441 | 1.935 | 4.040 |
| HC | G26H | 1.7955 | 0.545 | 1.1055 | 0.303 | 1.625 | 0.902 |
| HC | G26R | 1.9395 | 0.6055 | 1.444 | 0.338 | 1.342 | 1.793 |
| HC | G26D | 2.2105 | 0.7555 | 1.4155 | 0.4275 | 1.56 | 1.77 |
| HC | G26F | 0.588 | 0.2175 | 0.323 | 0.1345 | 1.822 | 1.628 |
| HC | G26M | 1.32 | 0.4535 | 0.841 | 0.2495 | 1.571 | 1.817 |
| HC | G26N | 2.9605 | 1.9525 | 2.99 | 1.2305 | 0.99 | 1.587 |
| HC | G26P | 1.001 | 0.4445 | 1.0425 | 0.309 | 0.977 | 1.441 |
| HC | G26Q | 2.45 | 0.8875 | 1.9265 | 0.5285 | 1.272 | 1.687 |
| HC | G26S | 2.226 | 0.7665 | 1.883 | 0.463 | 1.185 | 1.673 |
| HC | G26Y | 1.4695 | 0.447 | 0.8715 | 0.252 | 1.686 | 1.772 |
| HC | G26L | 1.015 | 0.312 | 0.64 | 0.2245 | 1.586 | 1.395 |
| HC | F27H | 1.488 | 0.342 | 0.817 | 0.243 | 1.823 | 1.418 |
| HC | F27R | 1.367 | 0.861 | 0.774 | 0.239 | 1.767 | 3.628 |
| HC | F27A | 2.936 | 2.213 | 2.241 | 0.769 | 1.310 | 2.880 |
| HC | F27D | 3.061 | 1.792 | 2.674 | 1.026 | 1.147 | 1.754 |
| HC | F27E | 2.792 | 1.306 | 2.418 | 0.910 | 1.155 | 1.435 |
| HC | F27G | 2.644 | 2.445 | 1.733 | 0.536 | 1.536 | 4.766 |
| HC | F27M | 2.935 | 1.233 | 1.980 | 0.405 | 1.483 | 3.047 |
| HC | F27P | 2.711 | 0.953 | 1.603 | 0.501 | 1.720 | 1.990 |
| HC | F27Q | 2.207 | 1.265 | 1.554 | 0.439 | 1.420 | 2.880 |
| HC | F27S | 1.898 | 0.508 | 0.918 | 0.253 | 2.067 | 2.014 |
| HC | F27T | 2.836 | 1.241 | 1.875 | 0.531 | 1.513 | 2.341 |
| HC | F27V | 1.419 | 0.712 | 0.614 | 0.190 | 2.311 | 3.752 |
| HC | F27W | 1.270 | 0.319 | 0.577 | 0.176 | 2.204 | 1.816 |
| HC | F27Y | 2.187 | 0.711 | 1.017 | 0.245 | 2.217 | 2.908 |
| HC | F27L | 2.492 | 0.784 | 1.562 | 0.478 | 1.595 | 1.639 |
| HC | S28K | 3.1285 | 2.125 | 2.927 | 1.176 | 1.069 | 1.804 |
| HC | S28H | 2.1735 | 0.7705 | 1.4715 | 0.4045 | 1.481 | 1.918 |
| HC | S28R | 2.9975 | 1.3625 | 2.5995 | 0.8495 | 1.153 | 1.604 |
| HC | S28A | 2.148 | 0.8335 | 1.468 | 0.3875 | 1.464 | 2.158 |
| HC | S28D | 1.97 | 0.7175 | 1.1875 | 0.3805 | 1.663 | 1.89 |
| HC | S28I | 2.8715 | 1.3185 | 2.2545 | 0.6505 | 1.273 | 2.022 |
| HC | S28M | 2.635 | 0.984 | 1.911 | 0.574 | 1.38 | 1.718 |
| HC | S28P | 2.6535 | 1.132 | 1.94 | 0.606 | 1.371 | 1.868 |
| HC | S28Q | 2.98 | 1.4105 | 2.4315 | 0.775 | 1.229 | 1.823 |
| HC | S28V | 3.1155 | 1.6905 | 2.79 | 1.0175 | 1.12 | 1.675 |
| HC | S28W | 3.1335 | 1.685 | 2.628 | 0.909 | 1.193 | 1.855 |
| HC | S28L | 2.4775 | 1.9575 | 1.863 | 0.563 | 1.331 | 3.481 |
| HC | L29K | 1.476 | 0.837 | 0.747 | 0.371 | 1.976 | 2.418 |
| HC | L29H | 1.329 | 0.717 | 0.661 | 0.264 | 2.020 | 2.714 |
| HC | L29A | 1.626 | 0.643 | 1.109 | 0.344 | 1.473 | 2.080 |
| HC | L29D | 0.504 | 0.232 | 0.329 | 0.164 | 1.531 | 1.409 |
| HC | L29G | 0.728 | 0.198 | 0.464 | 0.163 | 1.567 | 1.224 |
| HC | L29I | 2.250 | 1.661 | 2.020 | 0.893 | 1.121 | 1.864 |
| HC | L29M | 2.220 | 1.031 | 1.836 | 0.637 | 1.214 | 1.619 |
| HC | L29N | 0.352 | 0.326 | 0.253 | 0.149 | 1.390 | 1.254 |
| HC | L29S | 0.916 | 0.414 | 0.470 | 0.206 | 1.952 | 2.038 |
| HC | L29V | 0.975 | 0.516 | 0.543 | 0.287 | 1.796 | 1.800 |
| HC | T30H | 1.483 | 0.576 | 1.123 | 0.290 | 1.326 | 1.999 |
| HC | T30R | 1.646 | 0.808 | 1.487 | 0.412 | 1.110 | 1.961 |
| HC | T30D | 1.445 | 0.582 | 1.043 | 0.295 | 1.387 | 1.974 |
| HC | T30G | 1.130 | 0.455 | 0.925 | 0.257 | 1.222 | 1.776 |
| HC | T30I | 1.407 | 0.801 | 1.108 | 0.308 | 1.280 | 1.433 |
| HC | T30M | 1.241 | 0.454 | 1.054 | 0.221 | 1.191 | 2.061 |
| HC | T30N | 1.471 | 0.530 | 1.126 | 0.270 | 1.306 | 1.956 |
| HC | T30P | 1.341 | 0.405 | 0.936 | 0.263 | 1.432 | 1.544 |
| HC | T30S | 1.225 | 0.510 | 1.080 | 0.287 | 1.134 | 1.785 |
| HC | T30V | 1.210 | 0.521 | 1.130 | 0.246 | 1.074 | 2.113 |
| HC | T30W | 1.393 | 0.528 | 0.960 | 0.242 | 1.451 | 2.183 |
| HC | T30Y | 1.121 | 0.534 | 0.941 | 0.369 | 1.193 | 1.432 |
| HC | N31K | 3.216 | 2.270 | 2.256 | 0.713 | 1.433 | 3.242 |
| HC | N31H | 3.153 | 2.116 | 1.952 | 0.544 | 1.656 | 3.922 |
| HC | N31D | 2.946 | 1.227 | 1.746 | 0.424 | 1.687 | 2.891 |
| HC | N31E | 3.210 | 2.909 | 2.668 | 1.594 | 1.233 | 1.914 |
| HC | N31G | 3.218 | 1.917 | 2.566 | 0.760 | 1.254 | 2.529 |
| HC | N31I | 2.651 | 0.860 | 0.921 | 0.241 | 2.881 | 3.567 |
| HC | N31T | 3.102 | 0.773 | 2.226 | 0.567 | 1.394 | 1.364 |
| HC | N31V | 2.724 | 1.003 | 1.105 | 0.137 | 2.466 | 3.747 |
| HC | N31L | 2.920 | 0.983 | 1.990 | 0.575 | 1.467 | 1.713 |
| HC | Y32H | 1.011 | 0.488 | 0.684 | 0.248 | 1.483 | 1.963 |
| HC | Y32R | 1.253 | 0.454 | 1.049 | 0.280 | 1.194 | 1.616 |
| HC | Y32C | 0.667 | 0.256 | 0.405 | 0.182 | 1.645 | 1.408 |
| HC | Y32M | 1.035 | 0.368 | 0.756 | 0.237 | 1.366 | 1.556 |
| HC | Y32N | 0.837 | 0.447 | 0.524 | 0.121 | 1.604 | 1.707 |
| HC | Y32T | 0.705 | 0.296 | 0.435 | 0.176 | 1.624 | 1.685 |
| HC | Y32V | 0.767 | 0.216 | 0.518 | 0.223 | 1.484 | 0.967 |
| HC | Y32L | 0.793 | 0.299 | 0.550 | 0.169 | 1.443 | 1.787 |
| HC | G33E | 3.048 | 1.162 | 2.323 | 0.474 | 1.349 | 2.617 |
| HC | G33M | 2.472 | 0.669 | 1.904 | 0.537 | 1.305 | 1.246 |
| HC | G33S | 3.245 | 2.463 | 3.160 | 1.936 | 1.027 | 1.303 |
| HC | G33T | 2.346 | 0.748 | 1.959 | 0.714 | 1.226 | 1.038 |
| HC | G33Y | 0.121 | 0.106 | 0.123 | 0.097 | 0.982 | 1.095 |
| HC | V34A | 0.566 | 0.197 | 0.280 | 0.102 | 2.024 | 1.928 |
| HC | V34C | 0.756 | 0.432 | 0.798 | 0.164 | 0.950 | 2.625 |
| HC | V34I | 1.803 | 0.772 | 1.352 | 0.391 | 1.334 | 1.971 |
| HC | V34M | 1.219 | 0.681 | 0.925 | 0.331 | 1.320 | 2.069 |
| HC | V34P | 0.064 | 0.058 | 0.060 | 0.026 | 1.074 | 1.116 |
| HC | V34L | 1.105 | 0.429 | 0.772 | 0.206 | 1.434 | 2.118 |
| HC | H35I | 0.069 | 0.457 | 0.055 | 0.056 | 1.260 | 1.024 |
| HC | H35Q | 0.895 | 0.219 | 0.450 | 0.155 | 1.996 | 1.409 |
| HC | W36K | 0.062 | 0.056 | 0.056 | 0.028 | 1.111 | 1.002 |
| HC | W36A | 0.532 | 0.150 | 0.274 | 0.104 | 1.944 | 1.453 |
| HC | W36I | 1.421 | 0.791 | 1.241 | 0.495 | 1.148 | 1.600 |
| HC | W36V | 1.501 | 0.790 | 1.364 | 0.480 | 1.099 | 1.647 |
| HC | W36Y | 1.189 | 0.456 | 0.887 | 0.277 | 1.340 | 1.648 |
| HC | V50K | 0.105 | 0.118 | 0.101 | 0.101 | 1.040 | 1.170 |
| HC | V50H | 2.570 | 0.974 | 2.352 | 0.727 | 1.095 | 1.340 |
| HC | V50A | 3.196 | 1.613 | 2.597 | 1.019 | 1.233 | 1.582 |
| HC | V50D | 0.626 | 0.212 | 0.406 | 0.149 | 1.543 | 1.434 |
| HC | V50E | 0.400 | 0.146 | 0.339 | 0.134 | 1.181 | 1.086 |
| HC | V50G | 2.847 | 1.118 | 2.232 | 0.841 | 1.277 | 1.333 |
| HC | V50I | 1.551 | 0.414 | 0.555 | 0.182 | 2.795 | 2.298 |
| HC | V50N | 1.816 | 0.522 | 0.804 | 0.239 | 2.268 | 2.188 |
| HC | V50Q | 2.843 | 1.043 | 1.913 | 0.503 | 1.487 | 2.079 |
| HC | V50T | 3.264 | 2.695 | 3.246 | 2.339 | 1.005 | 1.153 |
| HC | V50L | 0.695 | 0.232 | 0.298 | 0.064 | 2.387 | 1.833 |
| HC | I51K | 1.861 | 0.635 | 1.068 | 0.288 | 1.764 | 2.207 |
| HC | I51H | 2.446 | 1.912 | 1.183 | 0.304 | 2.070 | 2.334 |
| HC | I51A | 3.027 | 1.178 | 1.436 | 0.346 | 2.378 | 3.590 |
| HC | I51C | 2.501 | 0.848 | 1.306 | 0.307 | 1.916 | 2.774 |
| HC | I51E | 0.879 | 0.283 | 0.491 | 0.184 | 1.791 | 1.537 |
| HC | I51G | 1.017 | 0.313 | 0.347 | 0.143 | 2.925 | 2.186 |
| HC | I51N | 2.508 | 0.797 | 1.240 | 0.302 | 2.026 | 2.641 |
| HC | I51Q | 3.286 | 1.967 | 2.878 | 0.877 | 1.142 | 2.255 |
| HC | I51S | 3.087 | 1.406 | 2.276 | 0.582 | 1.357 | 2.418 |
| HC | I51V | 3.312 | 2.820 | 3.310 | 1.602 | 1.001 | 1.761 |
| HC | I51Y | 0.997 | 0.301 | 0.626 | 0.203 | 1.592 | 1.477 |
| HC | I51L | 3.286 | 2.289 | 3.038 | 0.951 | 1.082 | 2.408 |
| HC | W52I | 0.855 | 0.249 | 0.392 | 0.148 | 2.183 | 1.690 |
| HC | W52N | 2.980 | 1.888 | 2.290 | 0.917 | 1.307 | 2.061 |
| HC | W52Y | 2.989 | 2.413 | 2.187 | 0.883 | 1.369 | 2.092 |
| HC | S53H | 3.290 | 2.779 | 3.202 | 1.848 | 1.027 | 1.504 |
| HC | S53R | 1.585 | 0.458 | 1.356 | 0.346 | 1.372 | 1.658 |
| HC | S53A | 3.441 | 3.325 | 3.360 | 2.616 | 1.024 | 1.299 |
| HC | S53C | 3.202 | 1.915 | 3.321 | 1.734 | 0.964 | 1.069 |
| HC | S53G | 3.389 | 3.289 | 3.381 | 2.854 | 1.002 | 1.153 |
| HC | S53I | 3.311 | 2.974 | 3.261 | 2.174 | 1.016 | 1.370 |
| HC | S53M | 3.210 | 1.689 | 3.018 | 1.025 | 1.068 | 1.659 |
| HC | S53P | 3.229 | 2.414 | 3.160 | 1.676 | 1.022 | 1.444 |
| HC | S53Q | 2.856 | 1.126 | 1.921 | 0.400 | 1.624 | 3.485 |
| HC | S53L | 3.298 | 2.391 | 3.295 | 1.757 | 1.001 | 1.472 |
| HC | S53T | 3.272 | 2.617 | 3.473 | 1.037 | 0.948 | 2.643 |
| HC | S53V | 3.315 | 2.305 | 3.321 | 1.652 | 0.998 | 1.406 |
| HC | S53Y | 3.377 | 2.797 | 3.235 | 1.999 | 1.044 | 1.398 |
| HC | G54H | 2.800 | 1.241 | 2.238 | 0.855 | 1.251 | 1.454 |
| HC | G54R | 2.341 | 0.748 | 1.702 | 0.518 | 1.376 | 1.446 |
| HC | G54A | 3.253 | 1.980 | 2.792 | 1.083 | 1.172 | 2.214 |
| HC | G54C | 1.636 | 0.346 | 1.055 | 0.238 | 1.551 | 1.452 |
| HC | G54D | 2.758 | 1.191 | 1.987 | 0.553 | 1.390 | 2.156 |
| HC | G54P | 2.336 | 0.773 | 1.320 | 0.370 | 1.772 | 2.089 |
| HC | G54S | 0.769 | 0.217 | 0.389 | 0.136 | 2.004 | 1.609 |
| HC | G55H | 3.289 | 1.916 | 2.919 | 0.957 | 1.132 | 2.085 |
| HC | G55R | 3.195 | 2.738 | 3.099 | 1.332 | 1.031 | 1.355 |
| HC | G55M | 3.076 | 1.452 | 2.727 | 0.766 | 1.131 | 1.889 |
| HC | G55S | 3.007 | 1.282 | 2.530 | 0.579 | 1.189 | 2.225 |
| HC | G55Y | 1.350 | 0.339 | 0.707 | 0.204 | 1.923 | 1.666 |
| HC | N56K | 2.941 | 1.283 | 2.775 | 1.030 | 1.059 | 1.246 |
| HC | N56A | 3.111 | 1.131 | 1.799 | 0.374 | 1.730 | 3.022 |
| HC | N56P | 1.322 | 1.332 | 0.880 | 0.235 | 1.525 | 1.408 |
| HC | N56S | 3.288 | 1.415 | 2.511 | 0.693 | 1.311 | 2.044 |
| HC | N56V | 3.021 | 1.201 | 2.660 | 0.867 | 1.136 | 1.385 |
| HC | N56G | 2.992 | 0.991 | 1.578 | 0.390 | 1.897 | 2.545 |
| HC | T57H | 3.064 | 1.040 | 1.792 | 0.457 | 1.711 | 2.276 |
| HC | T57R | 3.367 | 2.070 | 3.090 | 1.247 | 1.090 | 1.661 |
| HC | T57L | 3.316 | 1.923 | 2.903 | 1.052 | 1.143 | 1.827 |
| HC | T57A | 3.376 | 2.238 | 2.975 | 1.110 | 1.135 | 2.020 |
| HC | T57C | 3.287 | 1.693 | 2.703 | 0.814 | 1.216 | 2.088 |
| HC | T57D | 1.860 | 0.440 | 0.804 | 0.203 | 2.318 | 2.167 |
| HC | T57F | 3.414 | 2.680 | 3.125 | 1.839 | 1.093 | 1.458 |
| HC | T57M | 3.349 | 1.930 | 2.975 | 0.531 | 1.127 | 1.840 |
| HC | T57N | 3.125 | 1.170 | 2.145 | 0.537 | 1.459 | 2.182 |
| HC | T57Q | 3.359 | 1.699 | 2.774 | 0.792 | 1.211 | 2.147 |
| HC | T57W | 3.311 | 1.776 | 2.772 | 0.725 | 1.195 | 2.452 |
| HC | T57Y | 3.456 | 2.210 | 3.124 | 1.459 | 1.106 | 1.515 |
| HC | D58L | 1.607 | 0.742 | 2.044 | 0.579 | 0.786 | 1.314 |
| HC | D58G | 3.291 | 1.793 | 2.723 | 0.965 | 1.209 | 1.862 |
| HC | D58M | 2.134 | 0.790 | 1.507 | 0.545 | 1.451 | 1.449 |
| HC | D58N | 3.266 | 2.134 | 2.887 | 1.412 | 1.132 | 1.325 |
| HC | D58Q | 1.683 | 0.481 | 0.844 | 0.256 | 2.005 | 1.878 |
| HC | Y59H | 1.692 | 0.571 | 1.066 | 0.251 | 1.610 | 2.246 |
| HC | Y59R | 2.971 | 1.756 | 2.709 | 0.914 | 1.097 | 2.003 |
| HC | Y59A | 1.621 | 0.399 | 0.699 | 0.186 | 2.832 | 2.149 |
| HC | Y59C | 2.628 | 0.883 | 1.790 | 0.421 | 1.579 | 2.078 |
| HC | Y59D | 1.032 | 0.272 | 0.353 | 0.145 | 2.967 | 1.863 |
| HC | Y59E | 2.457 | 0.801 | 1.227 | 0.164 | 2.016 | 2.581 |
| HC | Y59G | 2.663 | 1.600 | 2.376 | 0.842 | 1.116 | 1.900 |
| HC | Y59I | 2.962 | 1.866 | 2.199 | 0.996 | 1.483 | 1.922 |
| HC | Y59P | 0.575 | 0.187 | 0.183 | 0.132 | 3.219 | 1.417 |
| HC | Y59Q | 2.915 | 1.383 | 2.283 | 0.557 | 1.277 | 2.480 |
| HC | Y59S | 2.891 | 1.523 | 2.571 | 0.732 | 1.128 | 2.070 |
| HC | Y59T | 3.059 | 1.678 | 2.585 | 0.702 | 1.184 | 2.510 |
| HC | Y59V | 2.561 | 0.945 | 1.685 | 0.417 | 1.743 | 2.247 |
| HC | Y59W | 2.886 | 1.247 | 2.089 | 0.496 | 1.382 | 2.708 |
| HC | N60K | 3.012 | 1.697 | 2.313 | 0.893 | 1.306 | 1.902 |
| HC | N60A | 3.104 | 1.847 | 2.729 | 0.958 | 1.140 | 1.935 |
| HC | N60C | 2.070 | 0.596 | 1.170 | 0.299 | 1.824 | 1.999 |
| HC | N60D | 0.196 | 0.800 | 0.113 | 0.089 | 1.736 | 1.142 |
| HC | N60F | 2.386 | 0.935 | 1.355 | 0.398 | 2.039 | 2.370 |
| HC | N60G | 2.647 | 0.944 | 1.537 | 0.407 | 1.831 | 2.323 |
| HC | N60P | 1.097 | 0.342 | 0.419 | 0.171 | 2.634 | 2.003 |
| HC | N60Q | 1.676 | 0.484 | 0.889 | 0.262 | 1.946 | 1.854 |
| HC | N60S | 2.148 | 0.696 | 1.104 | 0.299 | 1.953 | 2.362 |
| HC | N60T | 2.755 | 1.083 | 1.910 | 0.520 | 1.490 | 2.093 |
| HC | N60Y | 2.844 | 1.291 | 2.407 | 0.676 | 1.197 | 1.921 |
| HC | T61N | 3.043 | 1.882 | 2.603 | 0.936 | 1.176 | 2.012 |
| HC | T61Q | 2.187 | 0.731 | 1.372 | 0.188 | 1.591 | 1.974 |
| HC | P62G | 2.593 | 1.009 | 1.765 | 0.508 | 1.469 | 1.985 |
| HC | F63H | 3.170 | 2.002 | 2.715 | 0.773 | 1.168 | 2.592 |
| HC | F63R | 2.377 | 0.681 | 0.957 | 0.259 | 2.485 | 2.636 |
| HC | F63L | 3.150 | 1.606 | 2.218 | 0.627 | 1.421 | 2.560 |
| HC | F63A | 2.387 | 0.746 | 1.016 | 0.263 | 2.349 | 2.841 |
| HC | F63C | 0.911 | 0.242 | 0.272 | 0.112 | 3.440 | 2.160 |
| HC | F63D | 2.984 | 1.277 | 1.839 | 0.456 | 1.629 | 2.806 |
| HC | F63G | 2.914 | 1.094 | 1.516 | 0.401 | 1.951 | 2.767 |
| HC | F63M | 3.073 | 1.526 | 2.122 | 0.449 | 1.448 | 3.401 |
| HC | F63N | 2.284 | 0.672 | 1.240 | 0.156 | 1.843 | 2.201 |
| HC | F63Q | 2.906 | 1.180 | 1.622 | 0.373 | 1.794 | 3.164 |
| HC | F63S | 2.894 | 1.014 | 1.511 | 0.162 | 1.917 | 6.301 |
| HC | F63V | 3.032 | 1.585 | 2.090 | 0.477 | 1.451 | 3.338 |
| HC | T64R | 3.052 | 1.908 | 2.925 | 0.933 | 1.044 | 2.051 |
| HC | T64L | 3.052 | 2.189 | 2.814 | 1.108 | 1.093 | 1.976 |
| HC | T64C | 2.770 | 1.082 | 2.220 | 0.589 | 1.250 | 1.839 |
| HC | T64F | 0.165 | 0.087 | 0.084 | 0.089 | 1.974 | 0.985 |
| HC | T64G | 3.088 | 1.925 | 3.011 | 0.955 | 1.026 | 2.018 |
| HC | T64N | 0.232 | 0.132 | 0.092 | 0.087 | 2.550 | 1.516 |
| HC | T64Q | 1.555 | 0.542 | 0.952 | 0.253 | 1.641 | 2.150 |
| HC | T64V | 2.784 | 1.255 | 2.046 | 0.261 | 1.362 | 2.224 |
| HC | S65H | 3.222 | 2.639 | 3.201 | 1.556 | 1.007 | 1.704 |
| HC | S65R | 3.199 | 2.297 | 3.080 | 1.033 | 1.041 | 2.226 |
| HC | S65L | 3.302 | 2.824 | 3.272 | 1.846 | 1.009 | 1.530 |
| HC | S65C | 3.233 | 2.804 | 2.969 | 1.317 | 1.090 | 1.761 |
| HC | S65E | 3.256 | 2.320 | 3.089 | 1.304 | 1.054 | 1.779 |
| HC | S65F | 3.231 | 2.362 | 3.025 | 1.420 | 1.068 | 1.664 |
| HC | S65G | 3.337 | 2.992 | 3.335 | 2.388 | 1.000 | 1.253 |
| HC | S65I | 3.220 | 2.108 | 2.996 | 1.180 | 1.075 | 1.788 |
| HC | S65M | 3.102 | 1.898 | 2.758 | 0.940 | 1.125 | 2.018 |
| HC | S65N | 3.224 | 2.277 | 2.919 | 1.060 | 1.106 | 2.151 |
| HC | S65P | 2.795 | 1.197 | 1.892 | 0.466 | 1.479 | 2.568 |
| HC | S65Q | 3.193 | 2.250 | 2.951 | 1.100 | 1.082 | 2.055 |
| HC | S65T | 3.191 | 1.802 | 2.779 | 0.915 | 1.149 | 1.972 |
| HC | S65W | 3.227 | 2.510 | 3.114 | 1.514 | 1.037 | 1.662 |
| HC | S65Y | 3.322 | 2.816 | 3.201 | 1.928 | 1.038 | 1.462 |
| HC | R66L | 3.149 | 1.674 | 2.785 | 0.636 | 1.131 | 2.636 |
| HC | R66A | 2.441 | 1.026 | 2.008 | 0.491 | 1.217 | 2.091 |
| HC | R66C | 2.036 | 0.645 | 1.022 | 0.281 | 1.992 | 2.298 |
| HC | R66E | 1.775 | 0.595 | 1.089 | 0.316 | 1.627 | 1.889 |
| HC | R66F | 2.462 | 0.416 | 1.195 | 0.259 | 2.070 | 1.603 |
| HC | R66N | 3.065 | 1.089 | 2.343 | 0.658 | 1.308 | 1.655 |
| HC | R66P | 0.469 | 0.169 | 0.306 | 0.123 | 1.537 | 1.378 |
| HC | R66Q | 3.010 | 1.421 | 2.386 | 0.712 | 1.261 | 1.999 |
| HC | R66S | 2.805 | 0.994 | 1.945 | 0.414 | 1.444 | 2.404 |
| HC | R66T | 0.612 | 0.200 | 0.326 | 0.123 | 1.879 | 1.628 |
| HC | R66V | 3.198 | 1.703 | 3.077 | 0.525 | 1.039 | 1.530 |
| HC | R66G | 2.234 | 0.565 | 0.977 | 0.247 | 2.291 | 2.292 |
| HC | L67A | 2.784 | 1.152 | 1.921 | 0.487 | 1.449 | 2.377 |
| HC | L67C | 3.189 | 1.868 | 2.640 | 0.675 | 1.208 | 2.768 |
| HC | L67D | 0.113 | 0.086 | 0.085 | 0.079 | 1.343 | 1.078 |
| HC | L67E | 2.953 | 1.155 | 2.003 | 0.552 | 1.475 | 2.151 |
| HC | L67I | 2.974 | 1.183 | 1.920 | 0.461 | 1.548 | 2.579 |
| HC | L67M | 2.889 | 1.300 | 2.100 | 0.558 | 1.376 | 2.345 |
| HC | L67Q | 2.297 | 0.634 | 1.116 | 0.297 | 2.057 | 2.151 |
| HC | L67S | 3.114 | 1.560 | 2.496 | 0.646 | 1.248 | 2.418 |
| HC | L67T | 2.929 | 1.127 | 1.712 | 0.393 | 1.713 | 2.871 |
| HC | L67V | 2.755 | 0.875 | 1.330 | 0.346 | 2.072 | 2.529 |
| HC | L67Y | 3.171 | 1.933 | 2.840 | 0.454 | 1.117 | 2.152 |
| HC | S68K | 3.274 | 2.096 | 2.959 | 1.092 | 1.109 | 1.920 |
| HC | S68H | 3.269 | 2.602 | 3.284 | 1.358 | 0.995 | 1.918 |
| HC | S68R | 3.146 | 2.252 | 2.931 | 1.108 | 1.074 | 2.033 |
| HC | S68L | 3.054 | 1.591 | 2.441 | 0.645 | 1.251 | 2.471 |
| HC | S68C | 3.161 | 2.327 | 3.050 | 1.209 | 1.037 | 1.924 |
| HC | S68D | 3.228 | 1.835 | 2.822 | 0.413 | 1.144 | 2.303 |
| HC | S68E | 3.123 | 2.025 | 2.841 | 0.965 | 1.100 | 2.104 |
| HC | S68F | 0.256 | 0.128 | 0.137 | 0.093 | 1.863 | 1.379 |
| HC | S68G | 2.935 | 1.566 | 2.300 | 0.778 | 1.278 | 2.013 |
| HC | S68I | 3.209 | 1.895 | 2.834 | 0.788 | 1.132 | 2.404 |
| HC | S68N | 3.114 | 1.621 | 2.721 | 0.762 | 1.145 | 2.132 |
| HC | S68Q | 3.222 | 2.075 | 3.033 | 1.071 | 1.064 | 1.938 |
| HC | S68T | 3.310 | 2.716 | 3.261 | 1.779 | 1.015 | 1.532 |
| HC | S68V | 3.099 | 1.701 | 2.661 | 0.761 | 1.165 | 2.237 |
| HC | I69A | 0.429 | 0.133 | 0.242 | 0.086 | 1.773 | 1.542 |
| HC | I69C | 1.045 | 0.317 | 0.810 | 0.186 | 1.291 | 1.705 |
| HC | I69G | 0.112 | 0.133 | 0.085 | 0.062 | 1.312 | 1.147 |
| HC | I69Y | 0.523 | 0.157 | 0.340 | 0.132 | 1.538 | 1.194 |
| HC | N70H | 3.459 | 1.652 | 2.155 | 0.741 | 1.736 | 2.229 |
| HC | N70R | 1.720 | 0.369 | 0.689 | 0.206 | 2.997 | 1.792 |
| HC | N70L | 3.184 | 1.401 | 2.232 | 0.608 | 1.429 | 2.305 |
| HC | N70D | 1.788 | 0.523 | 0.817 | 0.257 | 2.242 | 2.036 |
| HC | N70E | 3.223 | 1.695 | 2.394 | 0.721 | 1.373 | 2.350 |
| HC | N70F | 3.263 | 2.109 | 2.985 | 1.368 | 1.095 | 1.557 |
| HC | N70G | 2.992 | 1.363 | 2.359 | 0.675 | 1.268 | 2.021 |
| HC | N70I | 3.240 | 1.310 | 1.934 | 0.575 | 1.862 | 2.278 |
| HC | N70P | 0.192 | 0.445 | 0.375 | 0.235 | 0.502 | 2.019 |
| HC | N70Q | 3.194 | 1.500 | 2.347 | 0.854 | 1.364 | 1.765 |
| HC | N70S | 3.247 | 2.088 | 2.937 | 0.496 | 1.105 | 2.094 |
| HC | N70T | 3.207 | 1.679 | 2.488 | 0.747 | 1.289 | 2.248 |
| HC | N70V | 0.241 | 2.063 | 2.833 | 1.232 | 0.085 | 1.677 |
| HC | N70Y | 3.152 | 1.553 | 2.029 | 0.788 | 1.888 | 1.980 |
| HC | K71H | 3.096 | 1.235 | 2.366 | 0.657 | 1.309 | 1.883 |
| HC | K71R | 2.741 | 0.871 | 1.745 | 0.462 | 1.571 | 1.888 |
| HC | K71L | 3.205 | 1.828 | 2.883 | 1.290 | 1.112 | 1.422 |
| HC | K71A | 1.772 | 0.457 | 1.075 | 0.320 | 1.649 | 1.430 |
| HC | K71C | 3.353 | 1.977 | 2.687 | 1.093 | 1.248 | 1.891 |
| HC | K71F | 3.342 | 1.506 | 3.119 | 1.260 | 1.072 | 1.195 |
| HC | K71G | 2.921 | 0.979 | 2.094 | 0.536 | 1.402 | 1.827 |
| HC | K71Q | 3.049 | 1.267 | 2.617 | 1.082 | 1.165 | 1.179 |
| HC | K71S | 3.114 | 1.168 | 2.534 | 0.688 | 1.237 | 1.716 |
| HC | K71T | 2.533 | 0.830 | 1.688 | 0.299 | 1.500 | 1.544 |
| HC | K71V | 3.160 | 1.663 | 2.787 | 0.929 | 1.134 | 1.790 |
| HC | K71W | 3.294 | 1.708 | 3.017 | 1.261 | 1.092 | 1.356 |
| HC | K71Y | 3.334 | 2.035 | 2.898 | 1.410 | 1.150 | 1.443 |
| HC | D72K | 3.108 | 1.388 | 2.427 | 1.747 | 1.281 | 0.795 |
| HC | D72H | 3.203 | 1.653 | 2.744 | 0.711 | 1.179 | 2.325 |
| HC | D72R | 3.355 | 2.011 | 3.182 | 0.938 | 1.055 | 2.144 |
| HC | D72L | 3.252 | 2.402 | 1.511 | 0.561 | 2.153 | 4.308 |
| HC | D72A | 2.976 | 1.272 | 3.026 | 1.109 | 0.982 | 1.415 |
| HC | D72G | 2.694 | 0.972 | 1.583 | 0.429 | 1.711 | 2.272 |
| HC | D72I | 3.200 | 1.798 | 2.711 | 0.827 | 1.182 | 2.179 |
| HC | D72M | 3.144 | 1.529 | 2.747 | 0.621 | 1.149 | 2.470 |
| HC | D72N | 3.303 | 1.878 | 2.982 | 0.927 | 1.112 | 2.026 |
| HC | D72Q | 3.157 | 2.535 | 2.782 | 0.790 | 1.137 | 2.402 |
| HC | D72S | 3.166 | 1.894 | 3.042 | 0.931 | 1.041 | 2.037 |
| HC | D72V | 3.241 | 2.071 | 3.115 | 1.044 | 1.041 | 1.987 |
| HC | D72W | 3.182 | 1.722 | 1.248 | 0.368 | 2.551 | 4.678 |
| HC | D72Y | 3.172 | 1.646 | 2.513 | 0.711 | 1.269 | 2.319 |
| HC | N73H | 3.095 | 1.105 | 2.128 | 0.423 | 1.455 | 2.618 |
| HC | N73R | 2.908 | 1.026 | 1.738 | 0.387 | 1.672 | 2.650 |
| HC | N73L | 3.179 | 1.682 | 2.800 | 0.883 | 1.137 | 1.917 |
| HC | N73A | 2.307 | 0.773 | 1.016 | 0.300 | 2.229 | 2.589 |
| HC | N73C | 3.111 | 1.210 | 2.023 | 0.483 | 1.558 | 2.506 |
| HC | N73G | 2.985 | 1.059 | 1.910 | 0.512 | 1.584 | 2.072 |
| HC | N73I | 3.336 | 2.124 | 3.024 | 1.005 | 1.107 | 2.116 |
| HC | N73M | 3.226 | 1.307 | 1.902 | 0.511 | 1.782 | 2.558 |
| HC | N73P | 2.396 | 0.732 | 1.262 | 0.359 | 1.913 | 2.036 |
| HC | N73Q | 3.055 | 1.153 | 2.047 | 0.221 | 1.494 | 2.850 |
| HC | N73S | 2.962 | 1.097 | 1.959 | 0.485 | 1.541 | 2.265 |
| HC | N73T | 2.752 | 1.024 | 1.951 | 0.544 | 1.404 | 1.896 |
| HC | N73V | 2.522 | 0.733 | 1.382 | 0.358 | 1.827 | 2.046 |
| HC | N73W | 2.294 | 0.718 | 1.278 | 0.342 | 1.783 | 2.100 |
| HC | N73Y | 3.150 | 1.234 | 2.165 | 0.464 | 1.455 | 2.656 |
| HC | S74K | 2.981 | 1.013 | 1.883 | 0.413 | 1.601 | 2.457 |
| HC | S74H | 3.070 | 1.253 | 1.963 | 0.476 | 1.579 | 2.634 |
| HC | S74R | 3.062 | 1.331 | 2.222 | 0.511 | 1.387 | 2.604 |
| HC | S74L | 3.292 | 2.221 | 3.205 | 1.053 | 1.027 | 2.110 |
| HC | S74A | 2.809 | 0.996 | 1.874 | 0.436 | 1.501 | 2.288 |
| HC | S74C | 2.721 | 0.882 | 1.705 | 0.347 | 1.619 | 2.544 |
| HC | S74D | 2.946 | 1.353 | 1.967 | 0.467 | 1.500 | 2.897 |
| HC | S74E | 3.001 | 1.279 | 2.213 | 0.444 | 1.358 | 2.892 |
| HC | S74G | 2.857 | 2.244 | 1.714 | 0.429 | 1.762 | 2.895 |
| HC | S74I | 2.986 | 1.082 | 2.151 | 0.495 | 1.388 | 2.194 |
| HC | S74M | 3.068 | 1.146 | 2.144 | 0.455 | 1.458 | 2.517 |
| HC | S74P | 3.196 | 1.545 | 2.503 | 0.615 | 1.280 | 2.511 |
| HC | S74T | 3.201 | 1.466 | 2.578 | 0.612 | 1.246 | 2.395 |
| HC | S74V | 3.242 | 1.928 | 3.245 | 0.910 | 0.999 | 2.118 |
| HC | S74Y | 2.854 | 0.982 | 1.605 | 0.337 | 1.866 | 2.919 |
| HC | K75H | 3.278 | 1.961 | 2.863 | 0.371 | 1.146 | 2.638 |
| HC | K75R | 3.111 | 1.259 | 2.012 | 0.479 | 1.559 | 2.639 |
| HC | K75L | 3.216 | 1.226 | 2.331 | 0.710 | 1.390 | 1.725 |
| HC | K75A | 2.879 | 1.070 | 1.846 | 0.428 | 1.570 | 2.504 |
| HC | K75C | 3.008 | 1.064 | 1.550 | 0.359 | 1.948 | 2.967 |
| HC | K75E | 3.070 | 1.191 | 2.020 | 0.523 | 1.560 | 2.279 |
| HC | K75F | 3.068 | 1.189 | 1.735 | 0.388 | 1.770 | 3.064 |
| HC | K75M | 2.776 | 0.884 | 1.342 | 0.362 | 2.076 | 2.450 |
| HC | K75Q | 3.200 | 1.533 | 2.319 | 0.526 | 1.384 | 2.914 |
| HC | K75T | 2.633 | 0.807 | 1.408 | 0.349 | 1.870 | 2.311 |
| HC | K75V | 2.908 | 0.939 | 1.435 | 0.325 | 2.032 | 2.962 |
| HC | K75W | 2.656 | 0.797 | 1.098 | 0.280 | 2.422 | 2.850 |
| HC | K75Y | 2.993 | 1.195 | 1.770 | 0.397 | 1.693 | 3.015 |
| HC | S76H | 2.719 | 0.806 | 1.324 | 0.300 | 2.054 | 2.694 |
| HC | S76R | 2.877 | 1.042 | 1.473 | 0.328 | 1.953 | 3.171 |
| HC | S76L | 2.187 | 0.500 | 0.830 | 0.215 | 2.636 | 2.323 |
| HC | S76A | 2.598 | 0.982 | 1.652 | 0.580 | 1.608 | 1.693 |
| HC | S76C | 2.490 | 0.855 | 1.304 | 0.339 | 1.910 | 2.537 |
| HC | S76D | 2.429 | 1.711 | 1.130 | 0.257 | 2.196 | 2.827 |
| HC | S76E | 3.053 | 1.236 | 1.893 | 0.457 | 1.615 | 2.706 |
| HC | S76F | 3.013 | 1.143 | 1.958 | 0.443 | 1.540 | 2.582 |
| HC | S76M | 2.936 | 1.267 | 1.924 | 0.458 | 1.527 | 2.767 |
| HC | S76P | 2.566 | 0.824 | 1.186 | 0.291 | 2.172 | 2.835 |
| HC | S76Q | 2.670 | 0.843 | 1.578 | 0.420 | 1.697 | 2.009 |
| HC | S76T | 2.515 | 0.805 | 1.182 | 0.268 | 2.133 | 3.024 |
| HC | S76Y | 2.788 | 0.921 | 1.393 | 0.344 | 2.004 | 2.685 |
| HC | Q77H | 3.135 | 1.285 | 2.396 | 0.640 | 1.310 | 2.008 |
| HC | Q77R | 2.600 | 1.185 | 1.976 | 0.618 | 1.344 | 1.957 |
| HC | Q77L | 2.256 | 0.589 | 0.937 | 0.234 | 2.408 | 2.520 |
| HC | Q77A | 3.109 | 1.370 | 2.320 | 0.532 | 1.343 | 2.577 |
| HC | Q77E | 3.162 | 1.660 | 2.729 | 0.331 | 1.159 | 2.647 |
| HC | Q77G | 2.148 | 0.548 | 0.843 | 0.216 | 2.551 | 2.545 |
| HC | Q77I | 2.653 | 0.784 | 1.189 | 0.292 | 2.232 | 2.690 |
| HC | Q77M | 2.489 | 0.861 | 1.213 | 0.289 | 2.108 | 2.989 |
| HC | Q77N | 3.002 | 1.184 | 1.800 | 0.471 | 1.668 | 2.516 |
| HC | Q77S | 2.791 | 1.085 | 1.936 | 0.496 | 1.441 | 2.193 |
| HC | Q77V | 3.246 | 1.643 | 2.722 | 0.633 | 1.193 | 2.597 |
| HC | Q77W | 1.891 | 0.537 | 0.880 | 0.243 | 2.149 | 2.209 |
| HC | Q77Y | 2.328 | 0.650 | 1.248 | 0.285 | 1.880 | 2.291 |
| HC | Y93H | 0.386 | 0.134 | 0.204 | 0.088 | 1.883 | 1.512 |
| HC | Y93V | 0.570 | 0.193 | 0.327 | 0.117 | 1.739 | 1.652 |
| HC | Y93W | 0.167 | 0.081 | 0.095 | 0.072 | 1.743 | 1.126 |
| HC | Y94R | 0.611 | 0.510 | 0.600 | 0.264 | 1.034 | 1.935 |
| HC | Y94L | 0.484 | 0.210 | 0.256 | 0.121 | 1.888 | 1.738 |
| HC | R97H | 1.065 | 0.411 | 0.502 | 0.219 | 2.148 | 1.884 |
| HC | R97W | 0.065 | 0.062 | 0.075 | 0.032 | 0.859 | 0.930 |
| HC | A98P | 1.057 | 0.812 | 0.619 | 0.386 | 1.709 | 1.755 |
| HC | L99N | 1.202 | 0.662 | 0.655 | 0.401 | 1.836 | 1.652 |
| HC | L99W | 1.312 | 1.114 | 0.926 | 0.350 | 1.417 | 1.659 |
| HC | T100H | 3.152 | 2.147 | 3.128 | 1.981 | 1.008 | 1.084 |
| HC | T100L | 3.133 | 1.851 | 2.685 | 1.361 | 1.167 | 1.364 |
| HC | T100A | 3.201 | 2.377 | 2.996 | 1.752 | 1.068 | 1.356 |
| HC | T100D | 2.957 | 0.907 | 2.741 | 0.868 | 1.079 | 1.046 |
| HC | T100I | 2.910 | 1.690 | 2.199 | 1.376 | 1.448 | 1.229 |
| HC | T100N | 3.070 | 1.883 | 2.895 | 1.350 | 1.060 | 1.398 |
| HC | T100P | 0.819 | 0.253 | 0.262 | 0.119 | 3.141 | 2.119 |
| HC | T100Q | 3.167 | 1.966 | 3.093 | 1.685 | 1.025 | 1.168 |
| HC | T100S | 3.166 | 1.748 | 2.953 | 0.816 | 1.072 | 2.142 |
| HC | T100V | 3.237 | 1.957 | 2.775 | 1.307 | 1.173 | 1.499 |
| HC | T100Y | 2.924 | 1.238 | 2.473 | 0.937 | 1.182 | 1.321 |
| HC | Y101H | 3.319 | 2.884 | 3.256 | 2.203 | 1.019 | 1.309 |
| HC | Y101E | 0.081 | 0.075 | 0.090 | 0.038 | 0.894 | 0.995 |
| HC | Y101F | 2.795 | 0.990 | 1.719 | 0.450 | 1.632 | 2.202 |
| HC | Y101M | 3.072 | 1.802 | 2.893 | 1.574 | 1.063 | 1.145 |
| HC | Y101W | 3.237 | 1.648 | 3.078 | 0.756 | 1.052 | 2.178 |
| HC | Y102R | 0.091 | 0.086 | 0.074 | 0.077 | 1.221 | 1.109 |
| HC | Y102C | 0.099 | 0.085 | 0.088 | 0.087 | 1.128 | 1.042 |
| HC | Y102D | 0.093 | 0.084 | 0.086 | 0.080 | 1.084 | 1.059 |
| HC | Y102I | 0.094 | 0.082 | 0.073 | 0.075 | 1.290 | 1.099 |
| HC | Y102N | 0.096 | 0.082 | 0.077 | 0.075 | 1.250 | 1.088 |
| HC | Y102W | 3.058 | 1.411 | 2.711 | 0.941 | 1.129 | 1.500 |
| HC | D103R | 0.134 | 0.093 | 0.115 | 0.098 | 1.168 | 0.942 |
| HC | D103L | 0.082 | 0.095 | 0.085 | 0.034 | 0.963 | 1.307 |
| HC | D103A | 3.114 | 0.281 | 2.833 | 1.442 | 1.099 | 0.195 |
| HC | D103C | 0.076 | 0.078 | 0.075 | 0.072 | 1.021 | 1.087 |
| HC | D103I | 0.109 | 0.091 | 0.087 | 0.091 | 1.254 | 1.006 |
| HC | D103P | 0.075 | 0.079 | 0.081 | 0.068 | 0.928 | 1.146 |
| HC | D103Q | 2.998 | 1.947 | 2.901 | 1.601 | 1.033 | 1.219 |
| HC | D103Y | 0.077 | 0.081 | 0.076 | 0.072 | 1.013 | 1.129 |
| HC | Y104H | 1.429 | 0.974 | 0.777 | 0.531 | 1.860 | 1.840 |
| HC | Y104L | 1.717 | 0.894 | 0.988 | 0.419 | 1.747 | 2.133 |
| HC | Y104D | 0.493 | 0.334 | 0.199 | 0.123 | 2.471 | 2.701 |
| HC | Y104F | 1.890 | 1.364 | 0.982 | 0.539 | 1.927 | 2.530 |
| HC | Y104I | 1.268 | 0.552 | 0.690 | 0.323 | 1.838 | 1.709 |
| HC | Y104M | 0.956 | 0.789 | 0.528 | 0.398 | 1.803 | 1.971 |
| HC | Y104S | 0.441 | 0.333 | 0.165 | 0.110 | 2.678 | 3.052 |
| HC | Y104V | 0.839 | 0.697 | 0.479 | 0.323 | 1.753 | 2.161 |
| HC | E105H | 0.061 | 0.059 | 0.060 | 0.030 | 1.021 | 0.997 |
| HC | E105T | 1.103 | 0.655 | 0.751 | 0.385 | 1.469 | 1.701 |
| HC | F106L | 1.149 | 0.640 | 0.712 | 0.357 | 1.618 | 1.816 |
| HC | F106V | 0.308 | 0.111 | 0.185 | 0.095 | 1.667 | 1.174 |
| HC | F106W | 1.076 | 0.399 | 0.748 | 0.229 | 1.420 | 1.749 |
| HC | F106Y | 1.705 | 0.929 | 1.699 | 0.530 | 1.008 | 1.753 |
| HC | A107K | 1.095 | 0.652 | 1.061 | 0.377 | 1.033 | 1.732 |
| HC | A107H | 1.208 | 0.830 | 1.208 | 0.468 | 1.014 | 1.776 |
| HC | A107R | 1.354 | 0.832 | 1.162 | 0.485 | 1.165 | 1.717 |
| HC | A107L | 1.244 | 0.841 | 0.799 | 0.227 | 1.560 | 1.874 |
| HC | A107C | 1.069 | 0.566 | 0.842 | 0.322 | 1.277 | 1.762 |
| HC | A107D | 0.952 | 0.485 | 0.587 | 0.271 | 1.624 | 1.787 |
| HC | A107E | 1.049 | 0.755 | 0.787 | 0.378 | 1.332 | 1.997 |
| HC | A107G | 1.161 | 0.776 | 0.923 | 0.424 | 1.258 | 1.830 |
| HC | A107N | 0.990 | 0.567 | 1.035 | 0.316 | 0.995 | 1.799 |
| HC | A107S | 1.071 | 0.680 | 1.153 | 0.388 | 0.954 | 1.755 |
| HC | A107T | 1.141 | 0.615 | 0.851 | 0.358 | 1.343 | 1.723 |
| HC | A107Y | 1.368 | 0.802 | 1.121 | 0.422 | 1.230 | 1.898 |
| HC | Y108K | 0.930 | 0.266 | 0.448 | 0.150 | 2.076 | 1.776 |
| HC | Y108H | 2.023 | 1.102 | 1.597 | 0.598 | 1.266 | 1.838 |
| HC | Y108R | 0.516 | 0.173 | 0.275 | 0.106 | 1.883 | 1.631 |
| HC | Y108L | 1.518 | 0.635 | 1.024 | 0.297 | 1.482 | 2.139 |
| HC | Y108C | 0.802 | 0.311 | 0.481 | 0.170 | 1.666 | 1.829 |
| HC | Y108F | 1.934 | 1.187 | 1.760 | 0.635 | 1.100 | 1.872 |
| HC | Y108I | 1.534 | 0.703 | 1.061 | 0.367 | 1.446 | 1.927 |
| HC | Y108N | 1.536 | 0.719 | 0.918 | 0.368 | 1.674 | 1.958 |
| HC | Y108S | 1.438 | 0.676 | 0.905 | 0.307 | 1.589 | 2.209 |
| HC | Y108T | 1.482 | 0.672 | 0.905 | 0.298 | 1.644 | 2.254 |
| HC | Y108V | 0.434 | 0.157 | 0.229 | 0.098 | 1.900 | 1.607 |
| HC | Y108W | 1.845 | 0.938 | 1.154 | 0.430 | 1.604 | 2.185 |
| HC | W109I | 0.919 | 0.266 | 0.470 | 0.151 | 1.957 | 1.755 |
| HC | W109M | 1.162 | 0.442 | 0.865 | 0.232 | 1.346 | 1.903 |
| HC | W109Y | 0.994 | 0.323 | 0.593 | 0.177 | 1.676 | 1.832 |
| HC | G110R | 0.069 | 0.062 | 0.077 | 0.037 | 0.972 | 0.850 |
| HC | G110A | 1.937 | 0.839 | 1.589 | 0.541 | 1.229 | 1.552 |
| HC | G110M | 0.100 | 0.068 | 0.053 | 0.064 | 1.875 | 1.058 |
| HC | G110P | 0.234 | 0.099 | 0.142 | 0.078 | 1.652 | 1.279 |
| HC | G110T | 1.117 | 0.371 | 0.774 | 0.234 | 1.442 | 1.594 |
| HC | Q111K | 3.167 | 1.888 | 2.878 | 1.122 | 1.101 | 1.693 |
| HC | Q111H | 2.442 | 0.722 | 1.412 | 0.363 | 1.729 | 1.992 |
| HC | Q111R | 2.940 | 1.110 | 2.019 | 0.507 | 1.456 | 2.192 |
| HC | Q111L | 2.960 | 1.155 | 2.111 | 0.542 | 1.403 | 2.132 |
| HC | Q111D | 2.881 | 1.072 | 2.046 | 0.503 | 1.417 | 2.132 |
| HC | Q111E | 3.087 | 1.497 | 2.422 | 0.649 | 1.275 | 2.311 |
| HC | Q111G | 2.853 | 1.136 | 2.115 | 0.568 | 1.351 | 1.998 |
| HC | Q111M | 1.621 | 0.420 | 0.776 | 0.093 | 2.094 | 2.197 |
| HC | Q111P | 2.558 | 0.817 | 1.423 | 0.369 | 1.797 | 2.211 |
| HC | Q111S | 2.912 | 1.292 | 2.334 | 0.588 | 1.250 | 2.204 |
| HC | Q111T | 3.156 | 2.059 | 2.713 | 1.020 | 1.163 | 2.018 |
| HC | Q111V | 0.928 | 0.287 | 0.389 | 0.143 | 2.426 | 2.021 |
| HC | Q111W | 2.633 | 0.820 | 1.533 | 0.366 | 1.721 | 2.241 |
| HC | Q111Y | 2.705 | 1.111 | 1.891 | 0.506 | 1.431 | 2.192 |
| HC | G112A | 1.008 | 0.276 | 0.609 | 0.168 | 1.657 | 1.645 |
| HC | G112N | 0.152 | 0.218 | 0.120 | 0.074 | 1.269 | 1.075 |
| HC | G112P | 1.396 | 0.443 | 1.154 | 0.293 | 1.210 | 1.515 |
| HC | G112S | 0.774 | 0.208 | 0.537 | 0.142 | 1.442 | 1.462 |
| HC | G112T | 0.195 | 0.085 | 0.129 | 0.072 | 1.509 | 1.169 |
| HC | G112Y | 0.176 | 0.080 | 0.114 | 0.068 | 1.565 | 1.172 |
| LC | D1W | 2.925 | 1.768 | 2.617 | 0.583 | 1.124 | 1.594 |
| LC | I2C | 2.076 | 1.460 | 1.622 | 0.332 | 1.284 | 1.475 |
| LC | I2V | 2.520 | 1.080 | 1.908 | 0.530 | 1.326 | 2.054 |
| LC | I2W | 1.308 | 0.324 | 0.909 | 0.092 | 1.448 | 3.539 |
| LC | L3D | 0.977 | 0.280 | 0.481 | 0.149 | 2.031 | 1.898 |
| LC | L3F | 1.085 | 0.313 | 0.495 | 0.178 | 2.194 | 1.784 |
| LC | L3G | 3.056 | 2.119 | 3.021 | 0.406 | 1.015 | 2.677 |
| LC | L3S | 1.494 | 0.390 | 0.760 | 0.219 | 1.967 | 1.780 |
| LC | L3T | 2.433 | 0.850 | 1.908 | 0.396 | 1.276 | 2.157 |
| LC | L3V | 2.544 | 1.051 | 2.034 | 0.294 | 1.258 | 3.578 |
| LC | L3W | 2.342 | 0.652 | 1.239 | 0.313 | 1.891 | 2.088 |
| LC | L3Y | 2.522 | 0.894 | 1.958 | 0.476 | 1.310 | 1.881 |
| LC | L3R | 3.123 | 1.858 | 3.257 | 0.799 | 0.959 | 2.324 |
| LC | L4C | 1.277 | 0.354 | 0.511 | 0.172 | 2.500 | 2.065 |
| LC | L4E | 2.282 | 0.635 | 0.992 | 0.268 | 2.301 | 2.374 |
| LC | L4F | 0.666 | 0.196 | 0.257 | 0.105 | 2.595 | 1.876 |
| LC | L4I | 2.044 | 0.594 | 0.954 | 0.244 | 2.141 | 2.445 |
| LC | L4P | 1.034 | 0.288 | 0.434 | 0.143 | 2.387 | 2.025 |
| LC | L4S | 0.714 | 0.207 | 0.286 | 0.108 | 2.496 | 1.928 |
| LC | L4T | 1.397 | 0.383 | 0.540 | 0.163 | 2.594 | 2.343 |
| LC | L4V | 1.497 | 0.413 | 0.573 | 0.085 | 2.614 | 2.559 |
| LC | L4W | 0.867 | 0.225 | 0.331 | 0.115 | 2.626 | 1.962 |
| LC | L4K | 0.917 | 0.249 | 0.363 | 0.122 | 2.555 | 2.042 |
| LC | L4H | 1.168 | 0.298 | 0.395 | 0.139 | 2.948 | 2.152 |
| LC | L4R | 2.025 | 0.583 | 0.817 | 0.229 | 2.507 | 2.543 |
| LC | T5A | 2.306 | 1.577 | 1.307 | 0.268 | 1.768 | 2.845 |
| LC | T5C | 1.929 | 0.521 | 0.747 | 0.192 | 2.622 | 2.719 |
| LC | T5D | 2.275 | 0.814 | 1.408 | 0.289 | 1.615 | 2.818 |
| LC | T5E | 2.809 | 1.421 | 2.377 | 0.555 | 1.182 | 2.564 |
| LC | T5F | 2.403 | 0.890 | 1.568 | 0.351 | 1.532 | 2.537 |
| LC | T5G | 2.079 | 0.697 | 1.277 | 0.267 | 1.629 | 2.608 |
| LC | T5N | 2.438 | 0.947 | 1.721 | 0.363 | 1.417 | 2.609 |
| LC | T5P | 1.226 | 0.364 | 0.584 | 0.171 | 2.098 | 2.127 |
| LC | T5S | 2.515 | 0.908 | 1.421 | 0.320 | 1.772 | 2.843 |
| LC | T5W | 2.195 | 0.701 | 1.131 | 0.246 | 1.943 | 2.854 |
| LC | T5L | 2.512 | 1.262 | 2.186 | 0.525 | 1.149 | 2.405 |
| LC | T5K | 2.558 | 0.944 | 1.638 | 0.370 | 1.562 | 2.556 |
| LC | T5H | 2.800 | 1.163 | 1.669 | 0.355 | 1.678 | 3.277 |
| LC | T5R | 2.633 | 1.328 | 1.846 | 0.423 | 1.428 | 3.143 |
| LC | R24A | 2.819 | 1.801 | 2.525 | 0.751 | 1.119 | 2.406 |
| LC | R24C | 2.004 | 0.612 | 1.021 | 0.249 | 1.965 | 2.460 |
| LC | R24F | 2.121 | 0.749 | 1.259 | 0.288 | 1.688 | 2.605 |
| LC | R24G | 1.023 | 0.297 | 0.396 | 0.133 | 2.599 | 2.237 |
| LC | R24L | 2.886 | 1.764 | 2.615 | 0.748 | 1.104 | 2.372 |
| LC | R24M | 2.880 | 2.141 | 2.619 | 0.562 | 1.100 | 1.749 |
| LC | R24S | 2.443 | 0.980 | 1.621 | 0.365 | 1.508 | 2.689 |
| LC | R24W | 2.019 | 0.655 | 1.111 | 0.261 | 1.816 | 2.512 |
| LC | R24Y | 2.557 | 1.315 | 2.221 | 0.545 | 1.152 | 2.413 |
| LC | A25C | 2.233 | 0.712 | 1.754 | 0.334 | 1.275 | 2.130 |
| LC | A25G | 2.406 | 1.123 | 2.373 | 0.568 | 1.014 | 1.986 |
| LC | A25L | 1.794 | 0.494 | 1.182 | 0.240 | 1.534 | 2.063 |
| LC | A25V | 2.351 | 1.883 | 1.718 | 0.463 | 1.370 | 1.926 |
| LC | S26A | 2.032 | 0.623 | 1.194 | 0.320 | 1.703 | 1.949 |
| LC | S26C | 1.490 | 0.370 | 0.672 | 0.204 | 2.232 | 1.814 |
| LC | S26D | 1.076 | 1.362 | 0.504 | 0.199 | 2.140 | 1.689 |
| LC | S26I | 1.847 | 0.549 | 1.137 | 0.297 | 1.642 | 1.850 |
| LC | S26M | 1.882 | 0.511 | 0.944 | 0.271 | 1.999 | 1.920 |
| LC | S26N | 2.649 | 1.069 | 2.006 | 0.472 | 1.325 | 2.269 |
| LC | S26V | 1.023 | 0.318 | 0.487 | 0.181 | 2.104 | 1.778 |
| LC | S26W | 1.416 | 0.394 | 0.640 | 0.211 | 2.215 | 1.871 |
| LC | S26L | 2.514 | 0.892 | 1.679 | 0.460 | 1.498 | 1.938 |
| LC | S26G | 2.563 | 1.076 | 1.773 | 0.470 | 1.448 | 2.293 |
| LC | S26H | 2.686 | 1.429 | 2.620 | 0.764 | 1.029 | 1.871 |
| LC | S26R | 0.578 | 0.206 | 0.310 | 0.166 | 1.869 | 1.245 |
| LC | Q27A | 2.910 | 1.942 | 2.602 | 0.970 | 1.118 | 2.002 |
| LC | Q27D | 2.850 | 1.856 | 2.682 | 0.962 | 1.064 | 1.940 |
| LC | Q27E | 2.980 | 1.656 | 2.752 | 0.774 | 1.084 | 2.141 |
| LC | Q27F | 3.022 | 1.396 | 2.597 | 0.684 | 1.164 | 2.044 |
| LC | Q27I | 3.166 | 2.049 | 2.605 | 1.092 | 1.216 | 1.881 |
| LC | Q27M | 3.076 | 1.975 | 2.485 | 0.917 | 1.243 | 2.153 |
| LC | Q27N | 2.816 | 1.768 | 2.563 | 0.976 | 1.099 | 1.811 |
| LC | Q27P | 1.967 | 1.368 | 1.815 | 0.645 | 1.128 | 2.123 |
| LC | Q27T | 3.165 | 2.567 | 2.919 | 0.861 | 1.085 | 1.506 |
| LC | S28A | 2.339 | 0.741 | 1.315 | 0.353 | 1.779 | 2.100 |
| LC | S28D | 2.972 | 1.878 | 2.403 | 0.971 | 1.268 | 1.964 |
| LC | S28N | 3.165 | 2.818 | 3.278 | 1.196 | 0.966 | 1.114 |
| LC | S28Q | 2.869 | 1.140 | 2.247 | 0.527 | 1.277 | 2.168 |
| LC | S28L | 1.871 | 0.518 | 1.004 | 0.256 | 1.859 | 2.022 |
| LC | S28K | 2.492 | 0.759 | 1.663 | 0.411 | 1.499 | 1.871 |
| LC | S28H | 2.843 | 1.108 | 2.146 | 0.484 | 1.325 | 2.293 |
| LC | I29A | 2.899 | 1.699 | 2.373 | 0.947 | 1.222 | 1.796 |
| LC | I29E | 2.217 | 0.833 | 1.193 | 0.437 | 1.862 | 1.908 |
| LC | I29F | 2.761 | 1.091 | 1.913 | 0.613 | 1.444 | 1.781 |
| LC | I29S | 2.910 | 1.745 | 1.779 | 0.627 | 1.742 | 3.037 |
| LC | I29T | 2.967 | 1.544 | 2.317 | 0.796 | 1.282 | 1.944 |
| LC | I29R | 0.124 | 1.528 | 0.294 | 0.140 | 0.422 | 0.673 |
| LC | G30A | 2.660 | 1.192 | 2.154 | 0.626 | 1.236 | 1.905 |
| LC | G30E | 3.158 | 1.981 | 2.865 | 1.070 | 1.109 | 1.852 |
| LC | G30F | 2.951 | 1.136 | 2.046 | 0.474 | 1.442 | 2.408 |
| LC | G30I | 2.653 | 1.210 | 2.221 | 0.642 | 1.195 | 1.885 |
| LC | G30M | 3.077 | 1.589 | 2.595 | 0.864 | 1.189 | 1.841 |
| LC | G30P | 2.643 | 1.034 | 1.826 | 0.541 | 1.447 | 1.911 |
| LC | G30Q | 2.855 | 1.151 | 2.261 | 0.608 | 1.263 | 1.895 |
| LC | G30S | 2.918 | 1.562 | 2.272 | 0.708 | 1.284 | 2.213 |
| LC | G30V | 2.539 | 0.882 | 1.477 | 0.406 | 1.720 | 2.172 |
| LC | G30Y | 2.270 | 0.630 | 1.137 | 0.244 | 1.998 | 2.586 |
| LC | G30L | 3.075 | 1.525 | 2.530 | 0.351 | 1.216 | 2.155 |
| LC | G30K | 2.747 | 0.945 | 1.681 | 0.385 | 1.634 | 2.456 |
| LC | G30H | 2.864 | 1.080 | 2.184 | 0.577 | 1.317 | 1.874 |
| LC | G30R | 2.634 | 1.078 | 2.126 | 0.529 | 1.239 | 2.041 |
| LC | T31A | 3.109 | 1.829 | 2.594 | 0.794 | 1.202 | 2.305 |
| LC | T31F | 2.585 | 1.954 | 1.545 | 0.378 | 1.673 | 2.444 |
| LC | T31G | 3.135 | 1.900 | 2.537 | 0.908 | 1.236 | 2.093 |
| LC | T31M | 3.168 | 2.090 | 2.724 | 0.921 | 1.163 | 2.270 |
| LC | T31S | 3.017 | 1.525 | 2.487 | 0.796 | 1.213 | 1.916 |
| LC | T31V | 3.059 | 1.618 | 2.684 | 0.843 | 1.140 | 1.923 |
| LC | T31W | 2.825 | 1.133 | 1.755 | 0.480 | 1.639 | 2.359 |
| LC | T31L | 2.910 | 1.274 | 2.135 | 0.647 | 1.365 | 1.969 |
| LC | T31K | 3.195 | 2.263 | 2.923 | 1.161 | 1.093 | 1.949 |
| LC | T31H | 3.172 | 2.169 | 3.026 | 1.098 | 1.049 | 1.976 |
| LC | N32G | 2.507 | 2.003 | 2.318 | 0.992 | 1.081 | 1.057 |
| LC | I33F | 2.150 | 0.712 | 1.647 | 0.362 | 1.306 | 1.971 |
| LC | I33G | 0.497 | 0.726 | 0.321 | 0.122 | 1.552 | 1.396 |
| LC | I33M | 2.452 | 0.922 | 1.788 | 0.471 | 1.391 | 1.957 |
| LC | I33T | 2.308 | 0.841 | 1.714 | 0.447 | 1.351 | 1.880 |
| LC | I33V | 2.684 | 1.395 | 2.296 | 0.674 | 1.171 | 2.089 |
| LC | I33H | 0.520 | 0.162 | 0.305 | 0.101 | 1.707 | 1.603 |
| LC | I48M | 3.195 | 2.000 | 2.971 | 0.998 | 1.076 | 2.004 |
| LC | I48S | 2.486 | 1.520 | 2.469 | 0.741 | 1.007 | 1.037 |
| LC | I48L | 3.126 | 1.720 | 2.560 | 0.804 | 1.221 | 2.142 |
| LC | I48K | 3.092 | 1.618 | 2.624 | 0.785 | 1.180 | 2.062 |
| LC | K49A | 3.111 | 2.465 | 3.143 | 1.634 | 0.990 | 1.508 |
| LC | K49E | 2.831 | 1.362 | 2.504 | 0.999 | 1.136 | 1.374 |
| LC | K49F | 2.953 | 1.733 | 2.622 | 0.910 | 1.126 | 1.904 |
| LC | K49G | 3.059 | 2.388 | 3.056 | 1.493 | 1.001 | 1.602 |
| LC | K49N | 2.967 | 2.078 | 2.833 | 1.037 | 1.048 | 2.009 |
| LC | K49Q | 3.070 | 2.336 | 2.908 | 1.708 | 1.058 | 1.376 |
| LC | K49S | 3.179 | 2.802 | 3.199 | 1.890 | 0.994 | 1.485 |
| LC | K49T | 3.161 | 2.528 | 3.076 | 1.343 | 1.028 | 1.884 |
| LC | K49V | 3.087 | 1.831 | 2.694 | 0.947 | 1.145 | 1.934 |
| LC | K49Y | 2.948 | 1.490 | 2.252 | 0.699 | 1.309 | 2.130 |
| LC | K49L | 2.767 | 2.365 | 2.614 | 1.220 | 1.060 | 1.459 |
| LC | K49H | 3.068 | 1.734 | 2.749 | 0.736 | 1.116 | 2.356 |
| LC | K49R | 3.091 | 2.911 | 3.020 | 2.277 | 1.023 | 1.278 |
| LC | A51T | 2.711 | 1.309 | 1.762 | 0.477 | 1.541 | 2.749 |
| LC | A51L | 2.611 | 1.889 | 2.090 | 0.781 | 1.250 | 1.595 |
| LC | S52A | 3.173 | 2.440 | 2.815 | 0.622 | 1.130 | 2.031 |
| LC | S52C | 2.145 | 0.676 | 1.079 | 0.250 | 1.995 | 2.709 |
| LC | S52D | 3.127 | 2.159 | 2.649 | 0.884 | 1.180 | 2.446 |
| LC | S52E | 2.874 | 1.773 | 2.495 | 0.691 | 1.152 | 2.569 |
| LC | S52G | 2.398 | 0.920 | 1.424 | 0.356 | 1.692 | 2.585 |
| LC | S52I | 2.301 | 0.928 | 1.450 | 0.358 | 1.599 | 2.598 |
| LC | S52M | 2.462 | 0.880 | 1.489 | 0.312 | 1.668 | 2.833 |
| LC | S52Q | 2.678 | 1.044 | 1.825 | 0.409 | 1.471 | 2.562 |
| LC | S52V | 2.799 | 1.545 | 2.259 | 0.639 | 1.240 | 2.420 |
| LC | S52W | 2.632 | 1.007 | 1.620 | 0.386 | 1.632 | 2.623 |
| LC | S52R | 3.133 | 1.934 | 2.294 | 0.746 | 1.367 | 2.604 |
| LC | S52K | 3.028 | 1.494 | 1.964 | 0.510 | 1.542 | 2.954 |
| LC | E53G | 0.182 | 0.173 | 0.122 | 0.076 | 1.605 | 1.489 |
| LC | S54M | 2.365 | 2.496 | 2.846 | 1.362 | 0.831 | 1.427 |
| LC | I55A | 2.591 | 1.923 | 2.689 | 0.728 | 0.964 | 1.318 |
| LC | I55F | 2.450 | 1.521 | 2.503 | 0.781 | 0.980 | 1.950 |
| LC | S56G | 3.158 | 2.562 | 2.991 | 1.497 | 1.056 | 1.719 |
| LC | S56L | 3.088 | 2.195 | 2.849 | 1.078 | 1.084 | 2.042 |
| LC | S56A | 3.072 | 2.332 | 3.031 | 1.328 | 1.015 | 1.759 |
| LC | S56C | 2.974 | 1.448 | 2.383 | 0.328 | 1.250 | 2.158 |
| LC | S56D | 3.060 | 1.994 | 2.841 | 1.165 | 1.077 | 1.711 |
| LC | S56E | 3.130 | 2.431 | 2.972 | 1.482 | 1.053 | 1.642 |
| LC | S56F | 3.095 | 2.008 | 2.961 | 1.102 | 1.046 | 1.824 |
| LC | S56N | 3.043 | 2.136 | 3.044 | 1.188 | 1.000 | 1.804 |
| LC | S56P | 3.120 | 2.744 | 3.119 | 2.194 | 1.000 | 1.251 |
| LC | S56Q | 3.136 | 2.242 | 2.999 | 1.207 | 1.046 | 1.858 |
| LC | S56V | 3.034 | 2.233 | 2.949 | 1.338 | 1.029 | 1.671 |
| LC | S56W | 3.044 | 1.944 | 2.720 | 0.978 | 1.119 | 1.988 |
| LC | S56H | 0.132 | 0.088 | 0.100 | 0.094 | 1.309 | 0.932 |
| LC | S56R | 3.035 | 1.896 | 2.681 | 0.952 | 1.132 | 1.992 |
| LC | S56K | 3.126 | 2.375 | 2.994 | 1.459 | 1.044 | 1.629 |
| LC | Y86F | 0.314 | 0.106 | 0.176 | 0.083 | 1.789 | 1.291 |
| LC | Y86M | 0.265 | 0.095 | 0.152 | 0.077 | 1.751 | 1.235 |
| LC | Y86H | 0.454 | 0.140 | 0.259 | 0.046 | 1.754 | 1.475 |
| LC | Y87L | 1.364 | 0.391 | 0.662 | 0.189 | 2.060 | 2.070 |
| LC | Y87C | 2.233 | 0.766 | 1.316 | 0.354 | 1.697 | 2.169 |
| LC | Y87D | 0.692 | 0.193 | 0.295 | 0.114 | 2.345 | 1.700 |
| LC | Y87F | 2.372 | 1.681 | 2.434 | 1.000 | 0.981 | 1.709 |
| LC | Y87G | 0.941 | 0.252 | 0.344 | 0.118 | 2.738 | 2.145 |
| LC | Y87I | 2.941 | 1.874 | 2.773 | 0.977 | 1.061 | 1.917 |
| LC | Y87N | 1.369 | 0.921 | 0.571 | 0.166 | 2.397 | 2.199 |
| LC | Y87P | 0.697 | 0.195 | 0.358 | 0.144 | 1.947 | 1.353 |
| LC | Y87S | 2.337 | 0.880 | 1.488 | 0.387 | 1.571 | 2.273 |
| LC | Y87T | 2.232 | 0.926 | 1.739 | 0.533 | 1.283 | 1.746 |
| LC | Y87V | 2.621 | 1.571 | 2.360 | 0.730 | 1.110 | 2.155 |
| LC | Y87W | 2.260 | 1.231 | 2.159 | 0.739 | 1.046 | 1.667 |
| LC | Y87K | 1.493 | 0.385 | 0.700 | 0.207 | 2.137 | 1.860 |
| LC | Y87H | 0.295 | 0.118 | 0.194 | 0.104 | 1.522 | 1.132 |
| LC | Y87R | 1.711 | 0.562 | 0.997 | 0.296 | 1.716 | 1.949 |
| LC | Q89E | 2.195 | 0.799 | 1.637 | 0.441 | 1.342 | 1.815 |
| LC | N91L | 0.334 | 0.124 | 0.162 | 0.087 | 2.064 | 1.421 |
| LC | N91A | 2.624 | 2.060 | 2.319 | 1.753 | 1.131 | 1.182 |
| LC | N91C | 2.633 | 1.226 | 2.163 | 0.790 | 1.219 | 1.553 |
| LC | N91I | 2.911 | 1.849 | 2.630 | 0.571 | 1.108 | 1.607 |
| LC | N91M | 2.428 | 1.480 | 2.182 | 1.132 | 1.114 | 1.308 |
| LC | N91S | 2.994 | 2.783 | 2.760 | 2.387 | 1.085 | 1.166 |
| LC | N91T | 2.831 | 1.991 | 2.546 | 1.387 | 1.113 | 1.435 |
| LC | N91V | 2.740 | 1.978 | 2.498 | 1.686 | 1.098 | 1.173 |
| LC | N91H | 2.919 | 1.694 | 2.691 | 0.876 | 1.085 | 1.940 |
| LC | N91R | 0.097 | 0.080 | 0.083 | 0.075 | 1.159 | 1.056 |
| LC | N92C | 2.942 | 1.540 | 2.633 | 1.142 | 1.118 | 1.349 |
| LC | N92D | 3.181 | 2.318 | 2.980 | 1.686 | 1.067 | 1.375 |
| LC | N92L | 2.733 | 1.469 | 2.812 | 0.741 | 0.972 | 1.983 |
| LC | N92M | 2.853 | 1.874 | 2.849 | 0.876 | 1.002 | 2.139 |
| LC | N92S | 2.560 | 1.897 | 2.199 | 1.094 | 1.165 | 1.733 |
| LC | N92T | 2.583 | 2.056 | 2.586 | 1.336 | 0.997 | 1.551 |
| LC | N92V | 3.125 | 2.740 | 3.058 | 1.851 | 1.022 | 1.484 |
| LC | N92W | 2.147 | 1.772 | 1.969 | 1.546 | 1.092 | 1.146 |
| LC | N92Y | 2.125 | 0.636 | 1.703 | 0.477 | 1.248 | 1.332 |
| LC | N92H | 3.094 | 2.440 | 2.804 | 1.941 | 1.103 | 1.257 |
| LC | N92K | 2.429 | 0.625 | 2.546 | 0.519 | 0.955 | 2.118 |
| LC | N92R | 3.085 | 1.643 | 2.966 | 0.691 | 1.040 | 2.377 |
| LC | N93T | 0.197 | 0.094 | 0.192 | 0.048 | 1.029 | 1.021 |
| LC | T96L | 3.174 | 1.793 | 2.779 | 0.701 | 1.176 | 2.936 |
| LC | T96C | 2.941 | 1.378 | 2.394 | 0.699 | 1.230 | 1.972 |
| LC | T96M | 2.899 | 1.972 | 2.673 | 0.834 | 1.084 | 1.673 |
| LC | T96V | 3.101 | 1.774 | 3.006 | 0.936 | 1.032 | 1.898 |
| LC | T97L | 2.411 | 0.799 | 1.773 | 0.399 | 1.366 | 2.004 |
| LC | T97A | 2.794 | 1.273 | 3.309 | 0.332 | 0.845 | 1.954 |
| LC | T97D | 1.749 | 0.461 | 1.007 | 0.237 | 1.737 | 1.951 |
| LC | T97G | 1.691 | 0.521 | 1.423 | 0.276 | 1.190 | 1.888 |
| LC | T97Q | 2.618 | 1.004 | 2.602 | 0.537 | 1.005 | 1.869 |
| LC | T97S | 2.108 | 0.545 | 1.884 | 0.260 | 1.119 | 2.095 |
| LC | T97V | 2.316 | 0.998 | 2.021 | 0.517 | 1.151 | 1.935 |
| LC | T97K | 2.211 | 0.892 | 2.542 | 0.491 | 0.870 | 1.816 |
| LC | T97R | 0.542 | 0.180 | 0.282 | 0.127 | 1.922 | 1.420 |
| LC | F98A | 0.999 | 0.296 | 0.635 | 0.096 | 1.573 | 1.537 |
| LC | F98M | 2.228 | 0.686 | 1.414 | 0.431 | 1.582 | 1.600 |
| LC | F98S | 1.532 | 0.467 | 1.079 | 0.299 | 1.422 | 1.560 |
| LC | F98V | 1.895 | 0.533 | 1.161 | 0.315 | 1.645 | 1.699 |
| LC | F98Y | 2.871 | 1.365 | 2.439 | 0.785 | 1.177 | 1.738 |
| LC | G99L | 0.578 | 0.164 | 0.310 | 0.096 | 1.864 | 1.713 |
| LC | G99D | 0.521 | 0.132 | 0.308 | 0.088 | 1.692 | 1.498 |
| LC | G99E | 0.496 | 0.166 | 0.300 | 0.126 | 1.655 | 1.324 |
| LC | G99F | 0.583 | 0.183 | 0.255 | 0.094 | 2.288 | 1.940 |
| LC | G99I | 0.480 | 0.141 | 0.293 | 0.100 | 1.645 | 1.420 |
| LC | G99M | 0.599 | 0.182 | 0.291 | 0.111 | 2.057 | 1.640 |
| LC | G99N | 0.611 | 0.154 | 0.373 | 0.124 | 1.639 | 1.235 |
| LC | G99S | 1.517 | 0.525 | 1.365 | 0.236 | 1.112 | 2.226 |
| LC | G99T | 1.203 | 0.307 | 0.812 | 0.173 | 1.488 | 1.783 |
| LC | G99V | 0.701 | 0.186 | 0.431 | 0.105 | 1.631 | 1.768 |
| LC | G99K | 0.360 | 0.120 | 0.203 | 0.042 | 1.793 | 1.481 |
| LC | G99H | 0.496 | 0.126 | 0.346 | 0.069 | 1.440 | 1.831 |
| LC | Q100C | 2.836 | 1.308 | 2.238 | 0.619 | 1.278 | 2.113 |
| LC | Q100D | 3.035 | 2.136 | 3.057 | 1.429 | 0.993 | 1.495 |
| LC | Q100E | 2.932 | 1.985 | 2.880 | 1.120 | 1.018 | 1.773 |
| LC | Q100F | 3.039 | 2.002 | 2.863 | 1.155 | 1.061 | 1.736 |
| LC | Q100I | 2.917 | 1.641 | 2.727 | 0.974 | 1.070 | 1.685 |
| LC | Q100M | 3.079 | 1.799 | 2.753 | 1.005 | 1.119 | 1.802 |
| LC | Q100N | 3.113 | 2.782 | 3.138 | 2.163 | 0.992 | 1.287 |
| LC | Q100P | 3.072 | 2.357 | 3.146 | 1.497 | 0.977 | 1.575 |
| LC | Q100T | 3.064 | 2.278 | 2.950 | 0.612 | 1.039 | 1.778 |
| LC | Q100V | 3.095 | 2.148 | 2.942 | 1.292 | 1.052 | 1.671 |
| LC | Q100W | 2.873 | 1.702 | 2.757 | 0.853 | 1.043 | 2.000 |
| LC | Q100Y | 3.170 | 2.395 | 3.173 | 1.671 | 0.999 | 1.439 |
| LC | Q100K | 3.076 | 2.031 | 2.852 | 1.110 | 1.078 | 1.834 |
| LC | Q100H | 3.096 | 2.050 | 2.942 | 1.261 | 1.053 | 1.628 |
| LC | Q100R | 2.930 | 1.902 | 2.831 | 1.030 | 1.035 | 1.846 |

^{a}HC = Heavy Chain; LC = Light Chain
^{b}V = variable

b. Determining Antibody Concentration

The antibody concentration was determined by anti-EGFR antibody quantitation ELISA. Briefly, plates were coated with 100 μL sEGFR-H6 (Sino Biologicals Inc, Cat#10001-H08H) antigen at 12 nM (1.32ug/mL) in PBS; washed three times with 250 μl/well of PBS; and blocked for 1 hour at room temperature with 250 μl of PBS with 5 mg/mL BSA. Serial dilutions of anti-EGFR-FLAG antibody standards (protein A column purified) were prepared in PBS with 5 mg/mL BSA. The starting antibody concentration was 100 ng/mL followed by 1:3 dilutions as specified. Test sample dilutions were prepared (1:3 dilutions), and 100 μl/well of standard and test sample were added to wells and incubated at room temperature for 1 hr. Plates were washed 3× with 250 μl/well of PBS with 5 mg/mL BSA. 100 μL/well rabbit anti-human IgG-Fc-HRP was added at 1:5000 (final concentration 0.2 μL/mL) dilution in PBS/5 mg/mL BSA. The plate was incubated for 1 hr at RT; washed 3× with 250 μl/well of PBS/5 mg/mL BSA. TMB Substrate was added and plates were read as described above.

c. Calculating Specific Activity

The specific activity (SA) was calculated by dividing the average OD value by the antibody concentration. The specific activity was then normalized to give a normalized specific activity (NSA) for each variant by dividing the specific activity of the variant anti-EGFR antibody by the specific activity of the reference FLAG-tagged anti-EGFR parental antibody. Table 33 sets forth the normalized specific activity of each identified variant set forth above at dilution 1 and dilution 2. The variant anti-EGFR antibodies with an NSA>0.4 at pH 6.0 and an NSA<0.4 at pH 7.4 were identified and selected for further analysis. The mutations of these identified antibodies are antibodies containing light chain (LC) mutations: L004C, L004V, S056H or N091V; and antibodies containing heavy chain (HC) mutations: V024I, V024L, S025C, S025G, S025I, S025Q, S025T, S025L, N031I, N031T, N031V, Y032seT, V050L, G054R, G054C, G054P, D058M, Y059E, F063R, F063C, F063G, F063M, F063V, T064N, T064V, S068F, S068Q, D072K, D072L, D072M, D072W, N073Q, S074H, S074R, S074D, S074G, S074Y, K075H, K075W, Q077R, Q077E, T100I, T100P, Y101W, Y104D, Y104F, Y104S or A107N.

TABLE 33
Normalized Specific Activity at pH 6.0 and
pH 7.4 of Variant Anti-EGFR Antibodies.

| | | Normalized Specific | | Normalized Specific | |
|---|---|---|---|---|---|
| | | Activity (NSA) (pH 6.0) | | Activity (NSA) (pH 7.4) | |
| Chain^{a} | Mutation | Dilution 1 | Dilution 2 | Dilution 1 | Dilution 2 |
| HC | T23K | 1.020 | 2.018 | 0.986 | 1.435 |
| HC | T23H | 1.889 | 5.343 | 1.940 | 3.453 |
| HC | T23R | 1.057 | 2.664 | 1.048 | 1.587 |
| HC | T23A | 1.238 | 2.459 | 1.304 | 1.297 |
| HC | T23C | 1.073 | 2.651 | 1.096 | 1.673 |
| HC | T23E | 1.255 | 2.947 | 1.406 | 1.751 |
| HC | T23G | 1.057 | 1.084 | 0.727 | 0.627 |
| HC | T23I | 1.097 | 2.851 | 1.126 | 1.975 |
| HC | T23M | 0.546 | 0.946 | 0.494 | 0.702 |
| HC | T23N | 1.062 | 2.207 | 0.888 | 1.504 |
| HC | T23P | 0.118 | 0.235 | 0.079 | 0.273 |
| HC | T23S | 0.750 | 2.908 | 1.087 | 1.902 |
| HC | T23V | 0.117 | 0.329 | 0.097 | 0.290 |
| HC | T23W | 3.143 | 5.871 | 2.981 | 2.441 |
| HC | T23L | 3.495 | 3.868 | 2.889 | 2.277 |
| HC | V24R | 0.103 | 0.096 | 0.092 | 0.413 |
| HC | V24A | 0.600 | 0.307 | 0.439 | 0.525 |
| HC | V24F | 0.861 | 0.233 | 0.428 | 0.622 |
| HC | V24G | 0.525 | 0.323 | 0.461 | 0.614 |
| HC | V24I | 1.049 | 0.305 | 0.381 | 0.568 |
| HC | V24M | 1.007 | 0.348 | 0.490 | 0.653 |
| HC | V24P | 2.209 | 0.566 | 0.766 | 1.234 |
| HC | V24S | 0.657 | 0.337 | 0.460 | 0.581 |
| HC | V24T | 1.001 | 0.315 | 0.430 | 0.543 |
| HC | V24L | 0.551 | 0.176 | 0.145 | 0.245 |
| HC | S25H | 0.389 | 0.227 | 0.167 | 0.207 |
| HC | S25R | 0.397 | 0.175 | 0.237 | 0.255 |
| HC | S25A | 0.344 | 0.239 | 0.238 | 0.310 |
| HC | S25C | 0.564 | 0.262 | 0.365 | 0.423 |
| HC | S25D | 0.364 | 0.203 | 0.267 | 0.351 |
| HC | S25E | 0.365 | 0.142 | 0.321 | 0.400 |
| HC | S25F | 0.350 | 0.196 | 0.323 | 0.450 |
| HC | S25G | 0.573 | 0.237 | 0.228 | 0.317 |
| HC | S25I | 0.556 | 0.397 | 0.374 | 0.464 |
| HC | S25M | 0.320 | 0.248 | 0.288 | 0.394 |
| HC | S25P | 2.362 | 0.630 | 0.673 | 1.444 |
| HC | S25Q | 1.319 | 0.347 | 0.335 | 0.493 |
| HC | S25T | 0.648 | 0.118 | 0.353 | 0.192 |
| HC | S25V | 0.300 | 0.229 | 0.221 | 0.381 |
| HC | S25L | 0.504 | 0.283 | 0.267 | 0.361 |
| HC | G26H | 2.071 | 3.143 | 1.536 | 2.105 |
| HC | G26R | 1.821 | 2.842 | 1.633 | 1.911 |
| HC | G26D | 1.456 | 2.488 | 1.123 | 1.696 |
| HC | G26F | 0.672 | 1.242 | 0.444 | 0.925 |
| HC | G26M | 1.674 | 2.875 | 1.284 | 1.905 |
| HC | G26N | 0.705 | 2.325 | 0.858 | 1.765 |
| HC | G26P | 1.501 | 3.333 | 1.883 | 2.791 |
| HC | G26Q | 1.275 | 2.310 | 1.208 | 1.657 |
| HC | G26S | 1.298 | 2.234 | 1.322 | 1.625 |
| HC | G26Y | 1.638 | 2.491 | 1.170 | 1.692 |
| HC | G26L | 4.237 | 6.512 | 3.218 | 5.644 |
| HC | F27H | 1.267 | 0.291 | 0.713 | 1.059 |
| HC | F27R | 1.285 | 0.810 | 0.747 | 1.151 |
| HC | F27A | 0.643 | 0.485 | 0.504 | 0.864 |
| HC | F27D | 0.572 | 0.335 | 0.513 | 0.984 |
| HC | F27E | 0.500 | 0.234 | 0.444 | 0.836 |
| HC | F27G | 0.777 | 0.718 | 0.522 | 0.808 |
| HC | F27M | 0.723 | 0.304 | 0.501 | 0.512 |
| HC | F27P | 0.834 | 0.293 | 0.506 | 0.790 |
| HC | F27Q | 0.872 | 0.500 | 0.630 | 0.890 |
| HC | F27S | 1.248 | 0.334 | 0.620 | 0.852 |
| HC | F27T | 0.710 | 0.311 | 0.482 | 0.681 |
| HC | F27V | 1.758 | 0.882 | 0.781 | 1.205 |
| HC | F27W | 1.371 | 0.344 | 0.639 | 0.972 |
| HC | F27Y | 0.857 | 0.278 | 0.409 | 0.493 |
| HC | F27L | 0.813 | 0.256 | 0.523 | 0.800 |
| HC | S28K | 0.671 | 2.281 | 0.757 | 1.520 |
| HC | S28H | 1.188 | 2.106 | 0.969 | 1.332 |
| HC | S28R | 0.978 | 2.223 | 1.022 | 1.669 |
| HC | S28A | 1.412 | 2.739 | 1.162 | 1.534 |
| HC | S28D | 0.602 | 1.096 | 0.437 | 0.700 |
| HC | S28I | 1.363 | 3.128 | 1.289 | 1.859 |
| HC | S28M | 2.402 | 4.485 | 2.098 | 3.151 |
| HC | S28P | 1.701 | 3.628 | 1.498 | 2.339 |
| HC | S28Q | 1.048 | 2.480 | 1.030 | 1.642 |
| HC | S28V | 0.972 | 2.638 | 1.049 | 1.913 |
| HC | S28W | 0.814 | 2.190 | 0.823 | 1.423 |
| HC | S28L | 2.175 | 8.593 | 1.970 | 2.977 |
| HC | L29K | 0.392 | 1.111 | 0.282 | 0.698 |
| HC | L29H | 0.338 | 0.911 | 0.238 | 0.475 |
| HC | L29A | 1.181 | 2.332 | 1.142 | 1.769 |
| HC | L29D | 0.388 | 0.893 | 0.359 | 0.897 |
| HC | L29G | 2.475 | 3.360 | 2.240 | 3.922 |
| HC | L29I | 0.406 | 1.498 | 0.517 | 1.142 |
| HC | L29M | 1.067 | 2.477 | 1.252 | 2.170 |
| HC | L29N | 0.845 | 3.909 | 0.860 | 2.538 |
| HC | L29S | 0.684 | 1.545 | 0.497 | 1.088 |
| HC | L29V | 0.352 | 0.930 | 0.278 | 0.735 |
| HC | T30H | 1.092 | 2.120 | 1.109 | 1.430 |
| HC | T30R | 0.592 | 1.454 | 0.718 | 0.994 |
| HC | T30D | 1.159 | 2.336 | 1.122 | 1.588 |
| HC | T30G | 0.749 | 1.508 | 0.822 | 1.140 |
| HC | T30I | 1.025 | 2.917 | 1.082 | 1.502 |
| HC | T30M | 0.700 | 1.280 | 0.797 | 0.834 |
| HC | T30N | 1.065 | 1.917 | 1.093 | 1.311 |
| HC | T30P | 1.064 | 1.607 | 0.996 | 1.397 |
| HC | T30S | 0.786 | 1.636 | 0.929 | 1.233 |
| HC | T30V | 0.728 | 1.567 | 0.912 | 0.991 |
| HC | T30W | 1.003 | 1.900 | 0.927 | 1.169 |
| HC | T30Y | 0.708 | 1.688 | 0.797 | 1.561 |
| HC | N31K | 0.352 | 0.248 | 0.253 | 0.400 |
| HC | N31H | 0.317 | 0.212 | 0.201 | 0.280 |
| HC | N31D | 0.794 | 0.331 | 0.483 | 0.587 |
| HC | N31E | 0.203 | 0.184 | 0.173 | 0.517 |
| HC | N31G | 0.506 | 0.301 | 0.414 | 0.613 |
| HC | N31I | 0.933 | 0.303 | 0.333 | 0.435 |
| HC | N31T | 0.503 | 0.125 | 0.370 | 0.472 |
| HC | N31V | 0.763 | 0.281 | 0.318 | 0.197 |
| HC | N31L | 0.659 | 0.222 | 0.461 | 0.665 |
| HC | Y32H | 0.609 | 1.470 | 0.552 | 1.002 |
| HC | Y32R | 0.943 | 1.707 | 1.059 | 1.413 |
| HC | Y32C | 0.703 | 1.348 | 0.573 | 1.284 |
| HC | Y32M | 0.878 | 1.559 | 0.859 | 1.345 |
| HC | Y32N | 0.500 | 1.335 | 0.420 | 0.483 |
| HC | Y32T | 0.455 | 0.953 | 0.376 | 0.759 |
| HC | Y32V | 0.742 | 1.043 | 0.672 | 1.448 |
| HC | Y32L | 1.385 | 2.612 | 1.287 | 1.974 |
| HC | G33E | 1.207 | 2.300 | 0.931 | 0.950 |
| HC | G33M | 2.556 | 3.457 | 1.992 | 2.807 |
| HC | G33S | 0.801 | 3.041 | 0.790 | 2.418 |
| HC | G33T | 1.148 | 1.830 | 0.970 | 1.767 |
| HC | G33Y | 1.133 | 4.983 | 1.165 | 4.590 |
| HC | V34A | 2.444 | 4.254 | 1.716 | 3.125 |
| HC | V34C | 0.372 | 1.062 | 0.558 | 0.573 |
| HC | V34I | 1.073 | 2.295 | 1.141 | 1.650 |
| HC | V34M | 0.434 | 1.212 | 0.467 | 0.835 |
| HC | V34P | 0.692 | 3.135 | 0.920 | 1.994 |
| HC | V34L | 1.696 | 3.294 | 1.682 | 2.238 |
| HC | H35I | 2.905 | 96.092 | 3.285 | 16.573 |
| HC | H35Q | 4.926 | 6.014 | 3.510 | 6.052 |
| HC | W36K | 0.617 | 2.808 | 0.790 | 1.992 |
| HC | W36A | 2.841 | 4.005 | 2.076 | 3.920 |
| HC | W36I | 0.588 | 1.638 | 0.729 | 1.452 |
| HC | W36V | 0.864 | 2.274 | 1.114 | 1.961 |
| HC | W36Y | 1.484 | 2.844 | 1.570 | 2.454 |
| HC | V50K | 0.043 | 0.244 | 0.042 | 0.212 |
| HC | V50H | 1.916 | 3.631 | 1.774 | 2.742 |
| HC | V50A | 1.390 | 3.507 | 1.143 | 2.243 |
| HC | V50D | 1.070 | 1.811 | 0.702 | 1.284 |
| HC | V50E | 1.239 | 2.253 | 1.061 | 2.100 |
| HC | V50G | 0.844 | 1.656 | 0.669 | 1.261 |
| HC | V50I | 4.049 | 5.405 | 1.466 | 2.398 |
| HC | V50N | 1.053 | 1.512 | 0.471 | 0.700 |
| HC | V50Q | 2.666 | 4.891 | 1.815 | 2.384 |
| HC | V50T | 0.738 | 3.047 | 0.743 | 2.675 |
| HC | V50L | 0.440 | 0.735 | 0.191 | 0.205 |
| HC | I51K | 2.359 | 4.025 | 1.370 | 1.844 |
| HC | I51H | 3.723 | 14.555 | 1.822 | 2.338 |
| HC | I51A | 1.534 | 2.984 | 0.736 | 0.886 |
| HC | I51C | 1.765 | 2.990 | 0.932 | 1.094 |
| HC | I51E | 3.724 | 5.988 | 2.106 | 3.947 |
| HC | I51G | 5.223 | 8.038 | 1.801 | 3.716 |
| HC | I51N | 2.514 | 3.995 | 1.257 | 1.529 |
| HC | I51Q | 0.877 | 2.625 | 0.777 | 1.184 |
| HC | I51S | 1.255 | 2.857 | 0.936 | 1.196 |
| HC | I51V | 0.562 | 2.392 | 0.568 | 1.375 |
| HC | I51Y | 5.123 | 7.721 | 3.253 | 5.278 |
| HC | I51L | 0.874 | 3.046 | 0.818 | 1.280 |
| HC | W52I | 0.965 | 1.402 | 0.751 | 1.419 |
| HC | W52N | 0.592 | 1.877 | 0.774 | 1.549 |
| HC | W52Y | 0.981 | 3.959 | 1.220 | 2.463 |
| HC | S53H | 0.417 | 1.759 | 0.410 | 1.184 |
| HC | S53R | 1.583 | 2.285 | 1.370 | 1.749 |
| HC | S53A | 0.348 | 1.680 | 0.344 | 1.338 |
| HC | S53C | 0.908 | 2.714 | 0.953 | 2.487 |
| HC | S53G | 0.223 | 1.081 | 0.225 | 0.949 |
| HC | S53I | 0.384 | 1.723 | 0.382 | 1.274 |
| HC | S53M | 1.206 | 3.172 | 1.147 | 1.947 |
| HC | S53P | 0.669 | 2.501 | 0.662 | 1.757 |
| HC | S53Q | 1.381 | 2.721 | 0.940 | 0.979 |
| HC | S53L | 0.900 | 3.262 | 0.910 | 2.425 |
| HC | S53T | 0.419 | 1.674 | 0.450 | 0.671 |
| HC | S53V | 0.545 | 1.894 | 0.552 | 1.373 |
| HC | S53Y | 0.344 | 1.426 | 0.334 | 1.031 |
| HC | G54H | 0.543 | 0.241 | 0.445 | 0.850 |
| HC | G54R | 0.462 | 0.148 | 0.345 | 0.524 |
| HC | G54A | 0.304 | 0.185 | 0.268 | 0.519 |
| HC | G54C | 0.567 | 0.120 | 0.375 | 0.423 |
| HC | G54D | 0.377 | 0.163 | 0.279 | 0.387 |
| HC | G54P | 0.530 | 0.175 | 0.307 | 0.430 |
| HC | G54S | 0.376 | 0.106 | 0.195 | 0.341 |
| HC | G55H | 0.391 | 1.139 | 0.590 | 0.968 |
| HC | G55R | 0.342 | 1.464 | 0.563 | 1.211 |
| HC | G55M | 0.338 | 0.797 | 0.509 | 0.715 |
| HC | G55S | 0.335 | 0.715 | 0.480 | 0.548 |
| HC | G55Y | 0.530 | 0.664 | 0.472 | 0.679 |
| HC | N56K | 0.782 | 1.706 | 0.754 | 1.399 |
| HC | N56A | 1.330 | 2.417 | 0.785 | 0.816 |
| HC | N56P | 4.302 | 21.666 | 2.925 | 3.906 |
| HC | N56S | 1.096 | 2.358 | 0.854 | 1.178 |
| HC | N56V | 0.938 | 1.863 | 0.843 | 1.374 |
| HC | N56G | 1.586 | 2.626 | 0.854 | 1.056 |
| HC | T57H | 1.322 | 2.243 | 0.789 | 1.007 |
| HC | T57R | 0.609 | 1.872 | 0.571 | 1.152 |
| HC | T57L | 0.795 | 2.304 | 0.711 | 1.287 |
| HC | T57A | 0.913 | 3.028 | 0.822 | 1.533 |
| HC | T57C | 0.868 | 2.235 | 0.729 | 1.097 |
| HC | T57D | 2.819 | 3.335 | 1.244 | 1.571 |
| HC | T57F | 0.477 | 1.873 | 0.446 | 1.312 |
| HC | T57M | 1.025 | 2.955 | 0.931 | 0.830 |
| HC | T57N | 1.244 | 2.328 | 0.872 | 1.091 |
| HC | T57Q | 1.019 | 2.576 | 0.859 | 1.226 |
| HC | T57W | 0.744 | 1.996 | 0.636 | 0.832 |
| HC | T57Y | 0.572 | 1.829 | 0.528 | 1.233 |
| HC | D58L | 0.494 | 1.142 | 0.642 | 0.909 |
| HC | D58G | 0.733 | 1.998 | 0.620 | 1.097 |
| HC | D58M | 0.527 | 0.975 | 0.380 | 0.687 |
| HC | D58N | 0.602 | 1.969 | 0.544 | 1.330 |
| HC | D58Q | 0.879 | 1.255 | 0.450 | 0.681 |
| HC | Y59H | 0.664 | 1.120 | 0.711 | 0.837 |
| HC | Y59R | 0.317 | 0.937 | 0.491 | 0.829 |
| HC | Y59A | 0.688 | 0.847 | 0.504 | 0.670 |
| HC | Y59C | 0.581 | 0.976 | 0.673 | 0.791 |
| HC | Y59D | 2.044 | 2.694 | 1.189 | 2.433 |
| HC | Y59E | 0.618 | 1.008 | 0.525 | 0.351 |
| HC | Y59G | 0.337 | 1.012 | 0.511 | 0.905 |
| HC | Y59I | 0.334 | 1.053 | 0.422 | 0.955 |
| HC | Y59P | 3.607 | 5.871 | 1.953 | 7.045 |
| HC | Y59Q | 0.438 | 1.039 | 0.583 | 0.710 |
| HC | Y59S | 0.340 | 0.895 | 0.514 | 0.731 |
| HC | Y59T | 0.395 | 1.082 | 0.567 | 0.770 |
| HC | Y59V | 0.559 | 1.032 | 0.625 | 0.773 |
| HC | Y59W | 0.488 | 1.054 | 0.600 | 0.712 |
| HC | N60K | 0.456 | 1.285 | 0.595 | 1.149 |
| HC | N60A | 0.432 | 1.286 | 0.646 | 1.134 |
| HC | N60C | 1.097 | 1.578 | 1.054 | 1.345 |
| HC | N60D | 2.085 | 42.638 | 2.049 | 8.023 |
| HC | N60F | 0.656 | 1.286 | 0.634 | 0.929 |
| HC | N60G | 0.669 | 1.192 | 0.660 | 0.873 |
| HC | N60P | 1.643 | 2.562 | 1.066 | 2.171 |
| HC | N60Q | 0.967 | 1.396 | 0.871 | 1.282 |
| HC | N60S | 0.722 | 1.169 | 0.631 | 0.853 |
| HC | N60T | 0.607 | 1.193 | 0.715 | 0.973 |
| HC | N60Y | 0.514 | 1.166 | 0.739 | 1.038 |
| HC | T61N | 0.436 | 1.348 | 0.634 | 1.140 |
| HC | T61Q | 0.719 | 1.201 | 0.766 | 0.524 |
| HC | P62G | 0.674 | 1.312 | 0.780 | 1.123 |
| HC | F63H | 0.498 | 0.315 | 0.438 | 0.624 |
| HC | F63R | 0.877 | 0.251 | 0.362 | 0.490 |
| HC | F63L | 0.736 | 0.375 | 0.532 | 0.752 |
| HC | F63A | 0.956 | 0.299 | 0.418 | 0.540 |
| HC | F63C | 1.107 | 0.294 | 0.339 | 0.699 |
| HC | F63D | 0.865 | 0.370 | 0.547 | 0.678 |
| HC | F63G | 0.746 | 0.280 | 0.398 | 0.527 |
| HC | F63M | 0.490 | 0.243 | 0.347 | 0.367 |
| HC | F63N | 2.041 | 0.601 | 1.138 | 0.713 |
| HC | F63Q | 0.732 | 0.297 | 0.419 | 0.482 |
| HC | F63S | 0.352 | 0.123 | 0.188 | 0.101 |
| HC | F63V | 0.514 | 0.269 | 0.364 | 0.415 |
| HC | T64R | 0.182 | 0.570 | 0.297 | 0.474 |
| HC | T64L | 0.298 | 1.068 | 0.467 | 0.919 |
| HC | T64C | 0.516 | 1.008 | 0.703 | 0.933 |
| HC | T64F | 0.693 | 1.832 | 0.598 | 3.168 |
| HC | T64G | 0.172 | 0.535 | 0.284 | 0.451 |
| HC | T64N | 0.493 | 1.405 | 0.331 | 1.574 |
| HC | T64Q | 0.540 | 0.941 | 0.562 | 0.746 |
| HC | T64V | 0.299 | 0.675 | 0.374 | 0.238 |
| HC | S65H | 0.325 | 1.329 | 0.335 | 0.814 |
| HC | S65R | 0.396 | 1.420 | 0.395 | 0.663 |
| HC | S65L | 0.139 | 0.596 | 0.143 | 0.404 |
| HC | S65C | 0.585 | 2.535 | 0.557 | 1.236 |
| HC | S65E | 0.508 | 1.810 | 0.500 | 1.056 |
| HC | S65F | 0.452 | 1.653 | 0.440 | 1.031 |
| HC | S65G | 0.254 | 1.139 | 0.264 | 0.944 |
| HC | S65I | 0.558 | 1.828 | 0.539 | 1.062 |
| HC | S65M | 0.556 | 1.700 | 0.513 | 0.874 |
| HC | S65N | 0.483 | 1.706 | 0.454 | 0.825 |
| HC | S65P | 0.974 | 2.084 | 0.684 | 0.842 |
| HC | S65Q | 0.492 | 1.734 | 0.472 | 0.880 |
| HC | S65T | 0.680 | 1.921 | 0.615 | 1.012 |
| HC | S65W | 0.380 | 1.476 | 0.380 | 0.924 |
| HC | S65Y | 0.329 | 1.393 | 0.329 | 0.990 |
| HC | R66L | 0.671 | 1.783 | 0.588 | 0.671 |
| HC | R66A | 0.998 | 2.099 | 0.814 | 0.996 |
| HC | R66C | 1.915 | 3.034 | 0.953 | 1.308 |
| HC | R66E | 1.811 | 3.037 | 1.102 | 1.597 |
| HC | R66F | 1.562 | 1.318 | 0.751 | 0.814 |
| HC | R66N | 0.935 | 1.661 | 0.709 | 0.995 |
| HC | R66P | 0.974 | 1.756 | 0.631 | 1.267 |
| HC | R66Q | 0.852 | 2.010 | 0.669 | 0.999 |
| HC | R66S | 0.900 | 1.594 | 0.619 | 0.658 |
| HC | R66T | 0.877 | 1.430 | 0.463 | 0.871 |
| HC | R66V | 0.936 | 2.491 | 0.893 | 0.761 |
| HC | R66G | 2.068 | 2.616 | 0.897 | 1.132 |
| HC | L67A | 0.980 | 2.026 | 0.702 | 0.889 |
| HC | L67C | 0.634 | 1.857 | 0.545 | 0.697 |
| HC | L67D | 0.033 | 0.125 | 0.026 | 0.120 |
| HC | L67E | 1.164 | 2.276 | 0.819 | 1.129 |
| HC | L67I | 0.879 | 1.748 | 0.589 | 0.706 |
| HC | L67M | 0.953 | 2.144 | 0.719 | 0.955 |
| HC | L67Q | 1.865 | 2.571 | 0.940 | 1.249 |
| HC | L67S | 0.925 | 2.316 | 0.769 | 0.996 |
| HC | L67T | 1.099 | 2.115 | 0.667 | 0.765 |
| HC | L67V | 1.443 | 2.291 | 0.723 | 0.940 |
| HC | L67Y | 0.852 | 2.598 | 0.792 | 0.633 |
| HC | S68K | 0.351 | 1.123 | 0.329 | 0.607 |
| HC | S68H | 1.426 | 5.677 | 1.487 | 3.076 |
| HC | S68R | 0.352 | 1.259 | 0.340 | 0.643 |
| HC | S68L | 0.862 | 2.245 | 0.715 | 0.945 |
| HC | S68C | 0.340 | 1.251 | 0.340 | 0.675 |
| HC | S68D | 0.661 | 1.879 | 0.600 | 0.439 |
| HC | S68E | 0.443 | 1.435 | 0.418 | 0.710 |
| HC | S68F | 0.713 | 1.782 | 0.396 | 1.344 |
| HC | S68G | 0.704 | 1.879 | 0.573 | 0.969 |
| HC | S68I | 0.608 | 1.796 | 0.558 | 0.775 |
| HC | S68N | 0.456 | 1.188 | 0.414 | 0.580 |
| HC | S68Q | 0.138 | 0.444 | 0.135 | 0.238 |
| HC | S68T | 0.288 | 1.183 | 0.295 | 0.805 |
| HC | S68V | 0.494 | 1.355 | 0.440 | 0.629 |
| HC | I69A | 2.116 | 3.280 | 1.694 | 3.009 |
| HC | I69C | 0.977 | 1.481 | 1.075 | 1.235 |
| HC | I69G | 2.582 | 15.344 | 2.793 | 10.186 |
| HC | I69Y | 1.759 | 2.635 | 1.624 | 3.153 |
| HC | N70H | 1.429 | 3.411 | 0.909 | 1.563 |
| HC | N70R | 3.154 | 3.380 | 1.291 | 1.930 |
| HC | N70L | 1.429 | 3.144 | 1.023 | 1.394 |
| HC | N70D | 3.706 | 5.415 | 1.729 | 2.720 |
| HC | N70E | 1.457 | 3.831 | 1.105 | 1.664 |
| HC | N70F | 0.867 | 2.802 | 0.810 | 1.857 |
| HC | N70G | 1.321 | 3.008 | 1.064 | 1.522 |
| HC | N70I | 1.764 | 3.564 | 1.075 | 1.597 |
| HC | N70P | 1.315 | 15.234 | 2.622 | 8.199 |
| HC | N70Q | 1.347 | 3.162 | 1.011 | 1.838 |
| HC | N70S | 1.059 | 3.405 | 0.978 | 0.825 |
| HC | N70T | 1.246 | 3.260 | 0.987 | 1.481 |
| HC | N70V | 0.071 | 3.033 | 0.851 | 1.850 |
| HC | N70Y | 1.291 | 3.180 | 0.849 | 1.649 |
| HC | K71H | 1.177 | 2.348 | 0.919 | 1.275 |
| HC | K71R | 1.746 | 2.774 | 1.135 | 1.501 |
| HC | K71L | 1.026 | 2.924 | 0.942 | 2.108 |
| HC | K71A | 2.515 | 3.244 | 1.558 | 2.316 |
| HC | K71C | 1.157 | 3.410 | 0.947 | 1.926 |
| HC | K71F | 0.767 | 1.728 | 0.731 | 1.477 |
| HC | K71G | 1.641 | 2.750 | 1.201 | 1.536 |
| HC | K71Q | 1.185 | 2.462 | 1.039 | 2.148 |
| HC | K71S | 1.258 | 2.360 | 1.046 | 1.419 |
| HC | K71T | 1.657 | 2.713 | 1.128 | 0.999 |
| HC | K71V | 1.077 | 2.833 | 0.970 | 1.617 |
| HC | K71W | 0.961 | 2.491 | 0.899 | 1.878 |
| HC | K71Y | 0.777 | 2.371 | 0.690 | 1.677 |
| HC | D72K | 0.483 | 1.078 | 0.382 | 1.374 |
| HC | D72H | 0.610 | 1.573 | 0.529 | 0.685 |
| HC | D72R | 0.299 | 0.897 | 0.287 | 0.424 |
| HC | D72L | 0.353 | 1.304 | 0.166 | 0.308 |
| HC | D72A | 0.760 | 1.625 | 0.783 | 1.435 |
| HC | D72G | 1.060 | 1.912 | 0.631 | 0.854 |
| HC | D72I | 0.528 | 1.484 | 0.453 | 0.691 |
| HC | D72M | 0.444 | 1.080 | 0.393 | 0.444 |
| HC | D72N | 0.699 | 1.986 | 0.639 | 0.992 |
| HC | D72Q | 0.547 | 2.197 | 0.488 | 0.693 |
| HC | D72S | 0.552 | 1.649 | 0.537 | 0.821 |
| HC | D72V | 0.435 | 1.389 | 0.423 | 0.709 |
| HC | D72W | 0.537 | 1.453 | 0.213 | 0.314 |
| HC | D72Y | 0.566 | 1.469 | 0.454 | 0.642 |
| HC | N73H | 1.037 | 1.850 | 0.722 | 0.716 |
| HC | N73R | 0.877 | 1.547 | 0.531 | 0.590 |
| HC | N73L | 0.661 | 1.749 | 0.590 | 0.930 |
| HC | N73A | 1.179 | 1.975 | 0.526 | 0.777 |
| HC | N73C | 1.032 | 2.006 | 0.680 | 0.810 |
| HC | N73G | 0.960 | 1.702 | 0.622 | 0.834 |
| HC | N73I | 0.551 | 1.755 | 0.506 | 0.841 |
| HC | N73M | 0.809 | 1.639 | 0.483 | 0.649 |
| HC | N73P | 1.410 | 2.152 | 0.752 | 1.070 |
| HC | N73Q | 0.898 | 1.693 | 0.609 | 0.329 |
| HC | N73S | 1.138 | 2.107 | 0.762 | 0.943 |
| HC | N73T | 1.090 | 2.026 | 0.782 | 1.090 |
| HC | N73V | 1.339 | 1.945 | 0.743 | 0.963 |
| HC | N73W | 1.193 | 1.868 | 0.673 | 0.901 |
| HC | N73Y | 0.901 | 1.764 | 0.627 | 0.672 |
| HC | S74K | 0.672 | 1.141 | 0.430 | 0.471 |
| HC | S74H | 0.612 | 1.249 | 0.396 | 0.480 |
| HC | S74R | 0.538 | 1.169 | 0.395 | 0.455 |
| HC | S74L | 0.425 | 1.435 | 0.419 | 0.689 |
| HC | S74A | 0.672 | 1.191 | 0.454 | 0.527 |
| HC | S74C | 0.718 | 1.164 | 0.456 | 0.464 |
| HC | S74D | 0.522 | 1.199 | 0.353 | 0.419 |
| HC | S74E | 0.729 | 1.554 | 0.545 | 0.546 |
| HC | S74G | 0.570 | 2.236 | 0.346 | 0.433 |
| HC | S74I | 0.648 | 1.174 | 0.473 | 0.544 |
| HC | S74M | 0.658 | 1.228 | 0.466 | 0.494 |
| HC | S74P | 0.566 | 1.369 | 0.449 | 0.552 |
| HC | S74T | 0.559 | 1.281 | 0.456 | 0.542 |
| HC | S74V | 0.386 | 1.147 | 0.391 | 0.548 |
| HC | S74Y | 0.692 | 1.191 | 0.394 | 0.414 |
| HC | K75H | 0.447 | 1.338 | 0.396 | 0.256 |
| HC | K75R | 0.688 | 1.393 | 0.451 | 0.537 |
| HC | K75L | 0.555 | 1.058 | 0.407 | 0.620 |
| HC | K75A | 0.732 | 1.360 | 0.475 | 0.550 |
| HC | K75C | 0.915 | 1.617 | 0.477 | 0.552 |
| HC | K75E | 0.791 | 1.535 | 0.527 | 0.683 |
| HC | K75F | 0.709 | 1.374 | 0.406 | 0.453 |
| HC | K75M | 0.841 | 1.338 | 0.412 | 0.555 |
| HC | K75Q | 0.611 | 1.463 | 0.448 | 0.508 |
| HC | K75T | 0.836 | 1.280 | 0.452 | 0.561 |
| HC | K75V | 1.060 | 1.712 | 0.530 | 0.599 |
| HC | K75W | 0.932 | 1.398 | 0.390 | 0.498 |
| HC | K75Y | 0.670 | 1.338 | 0.401 | 0.450 |
| HC | S76H | 1.102 | 1.634 | 0.544 | 0.615 |
| HC | S76R | 0.960 | 1.737 | 0.498 | 0.554 |
| HC | S76L | 1.414 | 1.615 | 0.543 | 0.704 |
| HC | S76A | 0.658 | 1.242 | 0.423 | 0.743 |
| HC | S76C | 1.207 | 2.072 | 0.640 | 0.831 |
| HC | S76D | 1.224 | 4.313 | 0.577 | 0.655 |
| HC | S76E | 1.121 | 2.268 | 0.704 | 0.849 |
| HC | S76F | 0.877 | 1.662 | 0.577 | 0.652 |
| HC | S76M | 0.841 | 1.815 | 0.558 | 0.665 |
| HC | S76P | 1.362 | 2.185 | 0.637 | 0.782 |
| HC | S76Q | 1.035 | 1.633 | 0.619 | 0.824 |
| HC | S76T | 1.702 | 2.724 | 0.810 | 0.918 |
| HC | S76Y | 1.264 | 2.087 | 0.639 | 0.788 |
| HC | Q77H | 0.760 | 1.558 | 0.588 | 0.785 |
| HC | Q77R | 0.480 | 1.093 | 0.369 | 0.577 |
| HC | Q77L | 1.600 | 2.089 | 0.673 | 0.839 |
| HC | Q77A | 0.869 | 1.914 | 0.656 | 0.753 |
| HC | Q77E | 0.684 | 1.794 | 0.598 | 0.362 |
| HC | Q77G | 1.626 | 2.074 | 0.646 | 0.826 |
| HC | Q077I | 1.339 | 1.978 | 0.608 | 0.745 |
| HC | Q77M | 0.987 | 1.708 | 0.487 | 0.579 |
| HC | Q77N | 1.063 | 2.097 | 0.646 | 0.845 |
| HC | Q77S | 0.792 | 1.539 | 0.556 | 0.712 |
| HC | Q77V | 0.701 | 1.773 | 0.595 | 0.692 |
| HC | Q77W | 1.272 | 1.805 | 0.600 | 0.828 |
| HC | Q77Y | 0.967 | 1.351 | 0.525 | 0.599 |
| HC | Y93H | 2.932 | 5.071 | 2.461 | 5.308 |
| HC | Y93V | 1.325 | 2.246 | 1.209 | 2.162 |
| HC | Y93W | 1.070 | 2.602 | 0.969 | 3.648 |
| HC | Y94R | 0.194 | 0.808 | 0.302 | 0.665 |
| HC | Y94L | 0.509 | 1.104 | 0.427 | 1.011 |
| HC | R97H | 0.853 | 1.644 | 0.638 | 1.390 |
| HC | R97W | 0.106 | 0.509 | 0.195 | 0.417 |
| HC | A98P | 0.284 | 1.092 | 0.265 | 0.824 |
| HC | L99N | 0.368 | 1.014 | 0.319 | 0.975 |
| HC | L99W | 0.259 | 1.101 | 0.291 | 0.550 |
| HC | T100H | 0.350 | 1.194 | 0.345 | 1.092 |
| HC | T100L | 0.545 | 1.609 | 0.463 | 1.172 |
| HC | T100A | 0.600 | 2.227 | 0.557 | 1.628 |
| HC | T100D | 0.551 | 0.846 | 0.507 | 0.802 |
| HC | T100I | 0.510 | 1.480 | 0.382 | 1.194 |
| HC | T100N | 0.535 | 1.642 | 0.500 | 1.166 |
| HC | T100P | 0.341 | 0.526 | 0.108 | 0.246 |
| HC | T100Q | 0.521 | 1.617 | 0.504 | 1.373 |
| HC | T100S | 0.441 | 1.218 | 0.408 | 0.564 |
| HC | T100V | 0.651 | 1.967 | 0.553 | 1.302 |
| HC | T100Y | 0.724 | 1.533 | 0.607 | 1.150 |
| HC | Y101H | 0.251 | 1.091 | 0.244 | 0.826 |
| HC | Y101E | 0.016 | 0.072 | 0.017 | 0.036 |
| HC | Y101F | 1.117 | 1.976 | 0.681 | 0.890 |
| HC | Y101M | 0.523 | 1.534 | 0.488 | 1.328 |
| HC | Y101W | 0.424 | 1.078 | 0.399 | 0.491 |
| HC | Y102R | 0.025 | 0.116 | 0.020 | 0.103 |
| HC | Y102C | 0.020 | 0.084 | 0.017 | 0.086 |
| HC | Y102D | 0.018 | 0.081 | 0.016 | 0.076 |
| HC | Y102I | 0.017 | 0.072 | 0.013 | 0.065 |
| HC | Y102N | 0.017 | 0.072 | 0.013 | 0.066 |
| HC | Y102W | 0.627 | 1.446 | 0.551 | 0.956 |
| HC | D103R | 0.014 | 0.050 | 0.012 | 0.053 |
| HC | D103L | 0.017 | 0.100 | 0.018 | 0.035 |
| HC | D103A | 0.669 | 0.302 | 0.604 | 1.536 |
| HC | D103C | 0.015 | 0.078 | 0.015 | 0.071 |
| HC | D103I | 0.158 | 0.657 | 0.124 | 0.648 |
| HC | D103P | 0.016 | 0.084 | 0.017 | 0.072 |
| HC | D103Q | 0.544 | 1.768 | 0.522 | 1.441 |
| HC | D103Y | 0.016 | 0.085 | 0.016 | 0.075 |
| HC | Y104H | 0.244 | 0.833 | 0.205 | 0.699 |
| HC | Y104L | 0.496 | 1.289 | 0.439 | 0.931 |
| HC | Y104D | 0.159 | 0.539 | 0.099 | 0.306 |
| HC | Y104F | 0.414 | 1.495 | 0.331 | 0.910 |
| HC | Y104I | 0.283 | 0.616 | 0.237 | 0.555 |
| HC | Y104M | 0.157 | 0.648 | 0.133 | 0.503 |
| HC | Y104S | 0.119 | 0.447 | 0.068 | 0.227 |
| HC | Y104V | 0.091 | 0.376 | 0.080 | 0.269 |
| HC | E105H | 0.149 | 0.720 | 0.226 | 0.555 |
| HC | E105T | 0.185 | 0.550 | 0.194 | 0.498 |
| HC | F106L | 0.266 | 0.740 | 0.253 | 0.636 |
| HC | F106V | 0.492 | 0.889 | 0.455 | 1.165 |
| HC | F106W | 0.537 | 0.997 | 0.575 | 0.879 |
| HC | F106Y | 0.579 | 1.578 | 0.889 | 1.385 |
| HC | A107K | 0.207 | 0.617 | 0.309 | 0.549 |
| HC | A107H | 0.182 | 0.624 | 0.280 | 0.541 |
| HC | A107R | 0.354 | 1.088 | 0.468 | 0.977 |
| HC | A107L | 0.552 | 1.866 | 0.546 | 0.774 |
| HC | A107C | 0.208 | 0.552 | 0.253 | 0.483 |
| HC | A107D | 0.237 | 0.604 | 0.225 | 0.520 |
| HC | A107E | 0.512 | 1.843 | 0.592 | 1.422 |
| HC | A107G | 0.263 | 0.877 | 0.321 | 0.738 |
| HC | A107N | 0.146 | 0.419 | 0.236 | 0.360 |
| HC | A107S | 0.258 | 0.818 | 0.428 | 0.720 |
| HC | A107T | 0.460 | 1.239 | 0.528 | 1.110 |
| HC | A107Y | 0.457 | 1.340 | 0.577 | 1.086 |
| HC | Y108K | 0.716 | 1.023 | 0.531 | 0.886 |
| HC | Y108H | 0.426 | 1.161 | 0.518 | 0.970 |
| HC | Y108R | 1.797 | 3.006 | 1.473 | 2.831 |
| HC | Y108L | 0.578 | 1.209 | 0.600 | 0.871 |
| HC | Y108C | 0.538 | 1.044 | 0.497 | 0.879 |
| HC | Y108F | 0.491 | 1.507 | 0.688 | 1.240 |
| HC | Y108I | 0.023 | 0.053 | 0.025 | 0.042 |
| HC | Y108N | 0.582 | 1.362 | 0.535 | 1.072 |
| HC | Y108S | 0.620 | 1.458 | 0.601 | 1.018 |
| HC | Y108T | 0.487 | 1.104 | 0.458 | 0.755 |
| HC | Y108V | 0.730 | 1.321 | 0.592 | 1.263 |
| HC | Y108W | 0.559 | 1.422 | 0.539 | 1.004 |
| HC | W109I | 1.412 | 2.041 | 1.112 | 1.788 |
| HC | W109M | 0.718 | 1.364 | 0.823 | 1.101 |
| HC | W109Y | 1.076 | 1.749 | 0.989 | 1.472 |
| HC | G110R | 1.165 | 5.194 | 1.833 | 4.433 |
| HC | G110A | 0.720 | 1.560 | 0.838 | 1.427 |
| HC | G110M | 3.249 | 11.020 | 2.455 | 14.823 |
| HC | G110P | 2.292 | 4.834 | 1.970 | 5.395 |
| HC | G110T | 1.168 | 1.938 | 1.149 | 1.733 |
| HC | Q111K | 0.603 | 1.796 | 0.568 | 1.108 |
| HC | Q111H | 2.478 | 3.661 | 1.487 | 1.909 |
| HC | Q111R | 1.220 | 2.302 | 0.870 | 1.091 |
| HC | Q111L | 1.349 | 2.631 | 0.999 | 1.282 |
| HC | Q111D | 1.207 | 2.244 | 0.889 | 1.094 |
| HC | Q111E | 1.067 | 2.588 | 0.869 | 1.163 |
| HC | Q111G | 1.342 | 2.672 | 1.033 | 1.386 |
| HC | Q111M | 2.871 | 3.716 | 1.426 | 0.855 |
| HC | Q111P | 2.397 | 3.826 | 1.384 | 1.795 |
| HC | Q111S | 1.171 | 2.598 | 0.974 | 1.227 |
| HC | Q111T | 0.760 | 2.478 | 0.678 | 1.274 |
| HC | Q111W | 1.646 | 2.561 | 0.994 | 1.186 |
| HC | Q111Y | 1.385 | 2.843 | 1.005 | 1.343 |
| HC | G112A | 2.121 | 2.898 | 1.723 | 2.371 |
| HC | G112N | 1.740 | 12.480 | 1.841 | 5.701 |
| HC | G112P | 1.475 | 2.338 | 1.641 | 2.080 |
| HC | G112S | 1.962 | 2.630 | 1.832 | 2.422 |
| HC | G112T | 2.850 | 6.175 | 2.537 | 7.080 |
| HC | G112Y | 2.473 | 5.622 | 2.147 | 6.430 |
| LC | D1W | 0.388 | 1.173 | 0.590 | 0.657 |
| LC | I2C | 0.739 | 2.597 | 0.981 | 1.004 |
| LC | I2V | 0.616 | 1.321 | 0.793 | 1.102 |
| LC | I2W | 0.715 | 0.885 | 0.844 | 0.427 |
| LC | L3D | 0.755 | 1.080 | 0.632 | 0.979 |
| LC | L3F | 0.811 | 1.170 | 0.629 | 1.131 |
| LC | L3G | 0.362 | 1.254 | 0.608 | 0.408 |
| LC | L3S | 0.668 | 0.872 | 0.578 | 0.833 |
| LC | L3T | 0.914 | 1.597 | 1.219 | 1.265 |
| LC | L3V | 0.968 | 1.999 | 1.315 | 0.949 |
| LC | L3W | 1.668 | 2.322 | 1.500 | 1.892 |
| LC | L3Y | 0.690 | 1.223 | 0.911 | 1.106 |
| LC | L3R | 0.337 | 1.002 | 0.597 | 0.733 |
| LC | L4C | 0.417 | 0.578 | 0.364 | 0.611 |
| LC | L4E | 1.177 | 1.636 | 1.114 | 1.502 |
| LC | L4F | 0.386 | 0.568 | 0.324 | 0.660 |
| LC | L4I | 0.508 | 0.738 | 0.517 | 0.661 |
| LC | L4P | 3.143 | 4.380 | 2.872 | 4.720 |
| LC | L4S | 0.594 | 0.862 | 0.518 | 0.975 |
| LC | L4T | 0.725 | 0.992 | 0.610 | 0.921 |
| LC | L4V | 0.444 | 0.613 | 0.371 | 0.275 |
| LC | L4W | 1.436 | 1.865 | 1.193 | 2.067 |
| LC | L4K | 2.048 | 2.777 | 1.767 | 2.957 |
| LC | L4H | 1.617 | 2.059 | 1.191 | 2.088 |
| LC | L4R | 1.694 | 2.439 | 1.489 | 2.087 |
| LC | T5A | 0.810 | 2.769 | 1.000 | 1.025 |
| LC | T5C | 1.935 | 2.614 | 1.631 | 2.098 |
| LC | T5D | 0.392 | 0.701 | 0.528 | 0.541 |
| LC | T5E | 0.508 | 1.285 | 0.936 | 1.092 |
| LC | T5F | 0.716 | 1.325 | 1.017 | 1.137 |
| LC | T5G | 0.756 | 1.267 | 1.011 | 1.058 |
| LC | T5N | 0.410 | 0.796 | 0.630 | 0.665 |
| LC | T5P | 0.296 | 0.440 | 0.308 | 0.450 |
| LC | T5S | 0.722 | 1.303 | 0.889 | 0.999 |
| LC | T5W | 0.866 | 1.382 | 0.972 | 1.055 |
| LC | T5L | 0.400 | 1.004 | 0.758 | 0.910 |
| LC | T5K | 0.652 | 1.202 | 0.909 | 1.026 |
| LC | T5H | 0.731 | 1.518 | 0.949 | 1.008 |
| LC | T5R | 0.436 | 1.099 | 0.665 | 0.762 |
| LC | R24A | 0.260 | 0.831 | 0.507 | 0.754 |
| LC | R24C | 1.592 | 2.429 | 1.766 | 2.150 |
| LC | R24F | 0.785 | 1.386 | 1.015 | 1.159 |
| LC | R24L | 0.494 | 1.508 | 0.974 | 1.393 |
| LC | R24M | 0.401 | 1.490 | 0.794 | 0.852 |
| LC | R24S | 0.968 | 1.941 | 1.399 | 1.573 |
| LC | R24W | 1.050 | 1.704 | 1.259 | 1.476 |
| LC | R24Y | 0.379 | 0.974 | 0.717 | 0.880 |
| LC | A25C | 1.571 | 2.505 | 2.125 | 2.024 |
| LC | A25G | 1.487 | 3.471 | 2.526 | 3.021 |
| LC | A25L | 2.166 | 2.983 | 2.459 | 2.496 |
| LC | A25V | 1.278 | 5.119 | 1.608 | 2.168 |
| LC | S26A | 0.864 | 1.323 | 0.863 | 1.156 |
| LC | S26C | 2.409 | 2.992 | 1.848 | 2.805 |
| LC | S26D | 1.717 | 10.871 | 1.367 | 2.694 |
| LC | S26I | 1.494 | 2.221 | 1.563 | 2.043 |
| LC | S26M | 1.137 | 1.543 | 0.969 | 1.392 |
| LC | S26N | 1.285 | 2.593 | 1.655 | 1.947 |
| LC | S26V | 1.567 | 2.436 | 1.269 | 2.357 |
| LC | S26W | 1.334 | 1.853 | 1.024 | 1.689 |
| LC | S26L | 1.073 | 1.903 | 1.219 | 1.668 |
| LC | S26G | 1.112 | 2.334 | 1.308 | 1.734 |
| LC | S26H | 0.557 | 1.482 | 0.924 | 1.348 |
| LC | S26R | 0.922 | 1.639 | 0.841 | 2.244 |
| LC | Q27A | 0.440 | 1.468 | 0.643 | 1.199 |
| LC | Q27D | 0.347 | 1.129 | 0.533 | 0.956 |
| LC | Q27E | 0.460 | 1.279 | 0.695 | 0.977 |
| LC | Q27F | 0.724 | 1.673 | 1.017 | 1.339 |
| LC | Q27I | 0.700 | 2.265 | 0.941 | 1.973 |
| LC | Q27M | 0.463 | 1.487 | 0.612 | 1.129 |
| LC | Q27N | 0.496 | 1.556 | 0.737 | 1.404 |
| LC | Q27P | 0.890 | 3.095 | 1.342 | 2.384 |
| LC | Q27T | 0.273 | 1.106 | 0.411 | 0.606 |
| LC | S28A | 0.930 | 1.472 | 0.889 | 1.193 |
| LC | S28D | 0.395 | 1.247 | 0.542 | 1.096 |
| LC | S28N | 0.233 | 1.039 | 0.411 | 0.749 |
| LC | S28Q | 0.350 | 0.696 | 0.466 | 0.547 |
| LC | S28L | 0.901 | 1.246 | 0.822 | 1.048 |
| LC | S28K | 0.753 | 1.147 | 0.855 | 1.056 |
| LC | S28H | 0.622 | 1.213 | 0.799 | 0.900 |
| LC | I29A | 0.426 | 1.249 | 0.570 | 1.137 |
| LC | I29E | 2.165 | 4.067 | 1.903 | 3.483 |
| LC | I29F | 1.438 | 2.842 | 1.628 | 2.610 |
| LC | I29S | 0.742 | 2.224 | 0.741 | 1.306 |
| LC | I29T | 0.575 | 1.496 | 0.733 | 1.260 |
| LC | I29R | 0.172 | 10.634 | 0.669 | 1.587 |
| LC | G30A | 0.643 | 1.440 | 0.851 | 1.236 |
| LC | G30E | 0.606 | 1.900 | 0.898 | 1.678 |
| LC | G30F | 0.938 | 1.806 | 1.064 | 1.232 |
| LC | G30I | 2.886 | 6.582 | 3.948 | 5.708 |
| LC | G30M | 0.594 | 1.535 | 0.819 | 1.364 |
| LC | G30P | 2.033 | 3.978 | 2.296 | 3.399 |
| LC | G30Q | 0.691 | 1.394 | 0.895 | 1.202 |
| LC | G30S | 0.885 | 2.369 | 1.126 | 1.755 |
| LC | G30V | 2.228 | 3.868 | 2.118 | 2.908 |
| LC | G30Y | 0.770 | 1.069 | 0.630 | 0.675 |
| LC | G30L | 1.918 | 4.755 | 2.578 | 1.786 |
| LC | G30K | 0.924 | 1.590 | 0.924 | 1.059 |
| LC | G30H | 0.649 | 1.224 | 0.809 | 1.069 |
| LC | G30R | 0.540 | 1.105 | 0.713 | 0.886 |
| LC | T31A | 0.581 | 1.710 | 0.793 | 1.213 |
| LC | T31F | 1.376 | 5.199 | 1.344 | 1.642 |
| LC | T31G | 0.617 | 1.869 | 0.816 | 1.460 |
| LC | T31M | 0.639 | 2.107 | 0.898 | 1.517 |
| LC | T31S | 0.646 | 1.633 | 0.870 | 1.393 |
| LC | T31V | 0.567 | 1.498 | 0.812 | 1.275 |
| LC | T31W | 0.831 | 1.665 | 0.843 | 1.153 |
| LC | T31L | 0.889 | 1.945 | 1.066 | 1.613 |
| LC | T31K | 0.370 | 1.312 | 0.554 | 1.100 |
| LC | T31H | 0.423 | 1.446 | 0.659 | 1.196 |
| LC | N32G | 0.315 | 1.257 | 0.501 | 1.072 |
| LC | I33F | 2.925 | 4.843 | 3.859 | 4.235 |
| LC | I33G | 2.950 | 21.553 | 2.553 | 4.840 |
| LC | I33M | 1.602 | 3.012 | 2.012 | 2.650 |
| LC | I33T | 1.451 | 2.643 | 1.855 | 2.419 |
| LC | I33V | 1.020 | 2.650 | 1.502 | 2.204 |
| LC | I33H | 2.665 | 4.156 | 2.098 | 3.474 |
| LC | I48M | 0.296 | 0.926 | 0.450 | 0.755 |
| LC | I48S | 0.380 | 1.161 | 0.617 | 0.925 |
| LC | I48L | 0.401 | 1.104 | 0.537 | 0.843 |
| LC | I48K | 0.406 | 1.063 | 0.564 | 0.843 |
| LC | K49A | 0.482 | 1.910 | 0.502 | 1.306 |
| LC | K49E | 1.256 | 3.021 | 1.146 | 2.287 |
| LC | K49F | 1.447 | 4.245 | 1.325 | 2.299 |
| LC | K49G | 0.658 | 2.569 | 0.679 | 1.657 |
| LC | K49N | 0.578 | 2.026 | 0.570 | 1.043 |
| LC | K49Q | 0.536 | 2.041 | 0.524 | 1.540 |
| LC | K49S | 0.362 | 1.597 | 0.376 | 1.112 |
| LC | K49T | 0.592 | 2.367 | 0.594 | 1.297 |
| LC | K49V | 1.002 | 2.973 | 0.903 | 1.586 |
| LC | K49Y | 1.231 | 3.109 | 0.970 | 1.505 |
| LC | K49L | 0.903 | 3.860 | 0.881 | 2.054 |
| LC | K49H | 0.912 | 2.578 | 0.843 | 1.128 |
| LC | K49R | 0.342 | 1.611 | 0.345 | 1.300 |
| LC | A51T | 0.980 | 2.366 | 1.388 | 1.877 |
| LC | A51L | 0.763 | 2.758 | 1.330 | 2.485 |
| LC | S52A | 0.398 | 1.531 | 0.770 | 0.850 |
| LC | S52C | 1.091 | 1.720 | 1.195 | 1.383 |
| LC | S52D | 0.499 | 1.721 | 0.920 | 1.535 |
| LC | S52E | 0.535 | 1.648 | 1.011 | 1.400 |
| LC | S52G | 0.519 | 0.996 | 0.672 | 0.840 |
| LC | S52I | 0.893 | 1.799 | 1.225 | 1.511 |
| LC | S52M | 1.055 | 1.884 | 1.390 | 1.453 |
| LC | S52Q | 0.770 | 1.500 | 1.142 | 1.279 |
| LC | S52V | 0.559 | 1.542 | 0.982 | 1.388 |
| LC | S52W | 0.877 | 1.678 | 1.176 | 1.399 |
| LC | S52R | 0.447 | 1.379 | 0.713 | 1.159 |
| LC | S52K | 0.689 | 1.700 | 0.974 | 1.264 |
| LC | E53G | 0.124 | 0.591 | 0.143 | 0.447 |
| LC | S54M | 0.196 | 1.032 | 0.394 | 0.943 |
| LC | I55A | 0.256 | 0.952 | 0.446 | 0.603 |
| LC | I55F | 0.445 | 1.383 | 0.762 | 1.190 |
| LC | S56G | 0.760 | 3.081 | 0.742 | 1.858 |
| LC | S56L | 0.934 | 3.319 | 0.889 | 1.682 |
| LC | S56A | 0.545 | 2.069 | 0.555 | 1.215 |
| LC | S56C | 1.565 | 3.809 | 1.294 | 0.889 |
| LC | S56D | 0.622 | 2.026 | 0.596 | 1.222 |
| LC | S56E | 0.523 | 2.033 | 0.513 | 1.279 |
| LC | S56F | 0.591 | 1.918 | 0.584 | 1.086 |
| LC | S56N | 0.555 | 1.949 | 0.573 | 1.119 |
| LC | S56P | 0.397 | 1.746 | 0.410 | 1.441 |
| LC | S56Q | 0.511 | 1.827 | 0.504 | 1.015 |
| LC | S56V | 0.563 | 2.070 | 0.564 | 1.279 |
| LC | S56W | 0.731 | 2.335 | 0.674 | 1.212 |
| LC | S56H | 0.414 | 1.379 | 0.325 | 1.528 |
| LC | S56R | 0.786 | 2.454 | 0.716 | 1.271 |
| LC | S56K | 0.476 | 1.808 | 0.470 | 1.146 |
| LC | Y86F | 2.224 | 3.759 | 1.675 | 3.937 |
| LC | Y86M | 2.183 | 3.912 | 1.679 | 4.267 |
| LC | Y86H | 1.595 | 2.459 | 1.224 | 1.087 |
| LC | Y87L | 1.922 | 2.752 | 0.963 | 1.374 |
| LC | Y87C | 1.722 | 2.953 | 1.047 | 1.408 |
| LC | Y87D | 3.099 | 4.313 | 1.364 | 2.624 |
| LC | Y87F | 0.376 | 1.332 | 0.398 | 0.817 |
| LC | Y87G | 3.312 | 4.426 | 1.249 | 2.134 |
| LC | Y87I | 0.609 | 1.941 | 0.593 | 1.044 |
| LC | Y87N | 2.778 | 9.344 | 1.195 | 1.738 |
| LC | Y87P | 2.621 | 3.657 | 1.389 | 2.794 |
| LC | Y87S | 1.480 | 2.785 | 0.972 | 1.264 |
| LC | Y87T | 1.323 | 2.744 | 1.064 | 1.629 |
| LC | Y87V | 0.736 | 2.206 | 0.684 | 1.058 |
| LC | Y87W | 0.793 | 2.161 | 0.782 | 1.338 |
| LC | Y87K | 2.610 | 3.360 | 1.262 | 1.867 |
| LC | Y87H | 0.236 | 0.470 | 0.160 | 0.429 |
| LC | Y87R | 1.388 | 2.280 | 0.834 | 1.237 |
| LC | Q89E | 0.961 | 1.749 | 1.031 | 1.387 |
| LC | N91L | 1.701 | 3.145 | 0.851 | 2.286 |
| LC | N91A | 0.275 | 1.079 | 0.251 | 0.948 |
| LC | N91C | 0.640 | 1.490 | 0.543 | 0.990 |
| LC | N91I | 0.678 | 2.153 | 0.632 | 0.685 |
| LC | N91M | 0.655 | 1.996 | 0.607 | 1.575 |
| LC | N91S | 0.363 | 1.688 | 0.345 | 1.494 |
| LC | N91T | 0.450 | 1.583 | 0.418 | 1.138 |
| LC | N91V | 0.401 | 1.446 | 0.377 | 1.272 |
| LC | N91H | 0.568 | 1.649 | 0.541 | 0.880 |
| LC | N91R | 0.029 | 0.121 | 0.026 | 0.118 |
| LC | N92C | 1.107 | 2.897 | 1.022 | 2.216 |
| LC | N92D | 0.819 | 2.986 | 0.792 | 2.241 |
| LC | N92L | 0.688 | 1.848 | 0.730 | 0.962 |
| LC | N92M | 0.576 | 1.892 | 0.593 | 0.912 |
| LC | N92S | 0.639 | 2.367 | 0.566 | 1.408 |
| LC | N92T | 0.735 | 2.923 | 0.759 | 1.960 |
| LC | N92V | 0.924 | 4.052 | 0.933 | 2.824 |
| LC | N92W | 0.563 | 2.322 | 0.532 | 2.091 |
| LC | N92Y | 1.771 | 2.649 | 1.465 | 2.052 |
| LC | N92H | 0.620 | 2.443 | 0.579 | 2.005 |
| LC | N92K | 0.635 | 0.817 | 0.687 | 0.700 |
| LC | N92R | 0.711 | 1.893 | 0.705 | 0.821 |
| LC | N93T | 0.035 | 0.085 | 0.050 | 0.062 |
| LC | T96L | 0.577 | 1.631 | 0.727 | 0.917 |
| LC | T96C | 1.341 | 3.139 | 1.570 | 2.292 |
| LC | T96M | 0.726 | 2.470 | 0.963 | 1.503 |
| LC | T96V | 0.476 | 1.360 | 0.663 | 1.032 |
| LC | T97L | 0.758 | 1.256 | 0.934 | 1.050 |
| LC | T97A | 0.430 | 0.978 | 0.852 | 0.427 |
| LC | T97D | 1.045 | 1.378 | 1.007 | 1.186 |
| LC | T97G | 0.722 | 1.113 | 1.018 | 0.988 |
| LC | T97Q | 0.616 | 1.181 | 1.025 | 1.058 |
| LC | T97S | 0.636 | 0.821 | 0.952 | 0.657 |
| LC | T97V | 0.629 | 1.355 | 0.919 | 1.176 |
| LC | T97K | 0.388 | 0.782 | 0.747 | 0.721 |
| LC | T97R | 0.859 | 1.424 | 0.748 | 1.681 |
| LC | F98A | 1.103 | 1.634 | 1.008 | 0.762 |
| LC | F98M | 1.483 | 2.284 | 1.354 | 2.064 |
| LC | F98S | 1.355 | 2.066 | 1.373 | 1.903 |
| LC | F98V | 2.045 | 2.874 | 1.802 | 2.446 |
| LC | F98Y | 0.548 | 1.302 | 0.669 | 1.077 |
| LC | G99L | 1.340 | 1.901 | 0.964 | 1.484 |
| LC | G99D | 1.750 | 2.208 | 1.387 | 1.970 |
| LC | G99E | 2.145 | 3.593 | 1.738 | 3.642 |
| LC | G99F | 4.569 | 7.151 | 2.674 | 4.939 |
| LC | G99I | 3.247 | 4.768 | 2.653 | 4.512 |
| LC | G99M | 3.359 | 5.103 | 2.188 | 4.173 |
| LC | G99N | 2.872 | 3.607 | 2.348 | 3.907 |
| LC | G99S | 0.960 | 1.660 | 1.158 | 0.999 |
| LC | G99T | 1.757 | 2.243 | 1.591 | 1.690 |
| LC | G99V | 2.850 | 3.773 | 2.351 | 2.864 |
| LC | G99K | 8.663 | 14.378 | 6.534 | 6.695 |
| LC | G99H | 1.416 | 1.792 | 1.323 | 1.311 |
| LC | Q100C | 1.256 | 2.897 | 1.023 | 1.414 |
| LC | Q100D | 0.772 | 2.716 | 0.802 | 1.875 |
| LC | Q100E | 0.797 | 2.698 | 0.808 | 1.570 |
| LC | Q100F | 0.716 | 2.360 | 0.696 | 1.404 |
| LC | Q100I | 1.154 | 3.244 | 1.113 | 1.986 |
| LC | Q100M | 0.867 | 2.533 | 0.800 | 1.460 |
| LC | Q100N | 0.472 | 2.107 | 0.491 | 1.691 |
| LC | Q100P | 0.736 | 2.823 | 0.778 | 1.850 |
| LC | Q100T | 0.870 | 3.235 | 0.864 | 0.896 |
| LC | Q100V | 0.672 | 2.331 | 0.659 | 1.447 |
| LC | Q100W | 0.791 | 2.343 | 0.783 | 1.211 |
| LC | Q100Y | 0.736 | 2.780 | 0.760 | 2.001 |
| LC | Q100K | 0.726 | 2.396 | 0.694 | 1.351 |
| LC | Q100H | 0.630 | 2.086 | 0.618 | 1.323 |
| LC | Q100R | 0.761 | 2.471 | 0.759 | 1.380 |

^{a}HC = Heavy Chain, LC = Light Chain

3. Confirmation Screen

A confirmation screen was performed as described in part 2, except that 5% serum was used at both pH values. Table 34 sets forth the OD at pH 6.0 (OD_{6.0}) at each dilution, OD at pH 7.4 (OD_{7.4}) at each dilution, and the ratio of the average OD values at pH 6.0 and 7.4 (OD_{6.0}/OD_{7.4}) for the exemplary tested modified antibodies at Dilution 1 and Dilution 2.

TABLE 34
Variant anti-EGFR antibodies

| | | OD_{pH 6.0} | | | | OD_{pH 7.4} | | | | OD_{pH 6.0}/OD_{pH 7.4} | |
|---|---|---|---|---|---|---|---|---|---|---|---|
| Chain^{a} | Mutation | Dilution 1 | | Dilution 2 | | Dilution 1 | | Dilution 2 | | Dilution 1 | Dilution 2 |
| HC | L029Y | 0.181 | 0.179 | 0.092 | 0.093 | 0.132 | 0.130 | 0.075 | 0.079 | 1.370 | 1.206 |
| HC | L029S | 1.116 | 1.143 | 0.303 | 0.327 | 0.545 | 0.583 | 0.074 | 0.074 | 2.004 | 4.251 |
| HC | L029K | 1.940 | 1.906 | 0.529 | 0.724 | 1.122 | 1.134 | 0.324 | 0.000 | 1.705 | 1.970 |
| HC | L029H | 0.814 | 0.790 | 0.216 | 0.203 | 0.396 | 0.355 | 0.132 | 0.124 | 2.142 | 1.635 |
| HC | L029N | 0.248 | 0.259 | 0.111 | 0.099 | 0.156 | 0.157 | 0.083 | 0.092 | 1.623 | 1.215 |
| HC | L029D | 0.543 | 0.496 | 0.156 | 0.149 | 0.285 | 0.290 | 0.104 | 0.115 | 1.808 | 1.401 |
| HC | L029V | 0.807 | 0.851 | 0.204 | 0.237 | 0.397 | 0.409 | 0.135 | 0.130 | 2.058 | 1.669 |
| HC | L029F | 0.420 | 0.445 | 0.131 | 0.137 | 0.251 | 0.244 | 0.105 | 0.108 | 1.748 | 1.255 |
| HC | L029I | 1.753 | 1.713 | 0.514 | 0.522 | 0.948 | 0.918 | 0.273 | 0.278 | 1.858 | 1.880 |
| HC | L029A | 1.822 | 1.861 | 0.582 | 0.662 | 0.938 | 0.888 | 0.275 | 0.289 | 2.019 | 2.202 |
| HC | L029M | 1.472 | 1.358 | 0.435 | 0.465 | 0.751 | 0.729 | 0.206 | 0.215 | 1.912 | 2.135 |
| HC | L029G | 0.291 | 0.289 | 0.111 | 0.111 | 0.171 | 0.165 | 0.089 | 0.093 | 1.723 | 1.226 |
| HC | T030V | 2.448 | 2.492 | 1.150 | 1.727 | 2.045 | 2.162 | 0.673 | 0.812 | 1.175 | 1.917 |
| HC | T030G | 2.528 | 2.483 | 1.323 | 1.329 | 1.748 | 1.659 | 0.557 | 0.000 | 1.471 | 2.399 |
| HC | T030S | 2.411 | 2.423 | 1.046 | 1.209 | 1.520 | 1.545 | 0.467 | 0.418 | 1.578 | 2.567 |
| HC | T030M | 2.189 | 2.256 | 0.713 | 0.900 | 1.289 | 1.230 | 0.338 | 0.322 | 1.766 | 2.453 |
| HC | T030R | 2.326 | 2.269 | 0.683 | 0.995 | 1.260 | 1.376 | 0.340 | 0.370 | 1.748 | 2.349 |
| HC | T030P | 2.217 | 2.216 | 0.688 | 0.911 | 1.261 | 1.159 | 0.318 | 0.375 | 1.836 | 2.298 |
| HC | T030H | 2.062 | 2.003 | 0.658 | 0.749 | 1.225 | 1.163 | 0.276 | 0.354 | 1.703 | 2.249 |
| HC | T030W | 2.042 | 2.058 | 0.683 | 0.702 | 1.208 | 1.139 | 0.316 | 0.322 | 1.748 | 2.170 |
| HC | T030D | 2.378 | 2.404 | 1.055 | 1.352 | 1.462 | 1.549 | 0.461 | 0.478 | 1.589 | 2.559 |
| HC | T030N | 2.276 | 2.237 | 0.907 | 1.027 | 1.396 | 1.326 | 0.373 | 0.410 | 1.659 | 2.470 |
| HC | Y032L | 1.263 | 1.249 | 0.307 | 0.349 | 0.570 | 0.567 | 0.182 | 0.161 | 2.209 | 1.932 |
| HC | Y032R | 1.871 | 1.881 | 0.513 | 0.554 | 0.994 | 0.980 | 0.279 | 0.259 | 1.901 | 1.987 |
| HC | Y032N | 2.023 | 2.042 | 0.611 | 2.097 | 0.992 | 1.090 | 0.365 | 0.359 | 1.957 | 1.832 |
| HC | Y032H | 2.097 | 2.099 | 0.804 | 0.841 | 1.199 | 1.229 | 0.397 | 0.396 | 1.729 | 2.074 |
| HC | Y032C | 0.426 | 0.451 | 0.137 | 0.168 | 0.378 | 0.401 | 0.140 | 0.137 | 1.125 | 1.104 |
| HC | Y032T | 1.927 | 1.880 | 0.732 | 0.963 | 1.333 | 1.316 | 0.582 | 0.643 | 1.437 | 1.378 |
| HC | Y032M | 2.356 | 2.411 | 0.557 | 0.825 | 1.622 | 1.604 | 0.462 | 0.504 | 1.478 | 1.423 |
| HC | V034L | 0.554 | 0.537 | 0.157 | 0.171 | 0.270 | 0.251 | 0.103 | 0.103 | 2.095 | 1.592 |
| HC | V034I | 1.200 | 1.061 | 0.317 | 1.455 | 0.533 | 0.503 | 0.147 | 0.163 | 2.183 | 2.076 |
| HC | V034M | 1.455 | 1.549 | 0.433 | 0.466 | 0.723 | 0.762 | 0.222 | 0.230 | 2.023 | 1.986 |
| HC | V034C | 1.317 | 1.265 | 0.359 | 0.357 | 0.667 | 0.652 | 0.197 | 0.192 | 1.956 | 1.845 |
| HC | H035N | 0.888 | 0.839 | 0.319 | 0.436 | 0.718 | 0.748 | 0.241 | 0.254 | 1.179 | 1.518 |
| HC | W036L | 1.601 | 1.562 | 0.456 | 0.495 | 0.629 | 0.599 | 0.201 | 0.207 | 2.578 | 2.332 |
| HC | W036Y | 0.608 | 0.574 | 0.171 | 0.176 | 0.242 | 0.224 | 0.101 | 0.108 | 2.533 | 1.662 |
| HC | I069T | 0.496 | 0.486 | 0.154 | 0.150 | 0.216 | 0.217 | 0.096 | 0.099 | 2.272 | 1.557 |
| HC | I069M | 0.498 | 0.509 | 0.148 | 0.181 | 0.216 | 0.239 | 0.096 | 0.100 | 2.218 | 1.673 |
| HC | I069C | 0.512 | 0.505 | 0.159 | 0.164 | 0.253 | 0.243 | 0.106 | 0.112 | 2.049 | 1.482 |
| HC | Y093H | 0.551 | 0.523 | 0.187 | 0.170 | 0.328 | 0.358 | 0.115 | 0.123 | 1.571 | 1.504 |
| HC | Y094L | 1.173 | 1.151 | 0.334 | 0.279 | 0.497 | 0.532 | 0.109 | 0.187 | 2.262 | 2.276 |
| HC | Y094R | 2.325 | 2.409 | 0.662 | 1.199 | 1.494 | 1.455 | 0.446 | 0.454 | 1.606 | 2.063 |
| HC | R097H | 1.077 | 1.150 | 0.211 | 0.360 | 0.406 | 0.425 | 0.139 | 0.143 | 2.679 | 2.019 |
| HC | L099N | 2.052 | 2.205 | 0.704 | 0.695 | 1.227 | 1.220 | 0.265 | 0.524 | 1.739 | 2.209 |
| HC | Y104S | 0.695 | 0.801 | 0.211 | 0.280 | 0.117 | 0.111 | 0.075 | 0.084 | 6.562 | 3.059 |
| HC | Y104V | 2.078 | 2.153 | 0.687 | 0.947 | 1.288 | 1.358 | 0.421 | 0.512 | 1.599 | 1.741 |
| HC | Y104D | 1.486 | 1.505 | 0.580 | 0.724 | 0.439 | 0.413 | 0.111 | 0.196 | 3.515 | 4.457 |
| HC | Y104M | 1.847 | 1.883 | 0.670 | 1.043 | 0.899 | 0.852 | 0.316 | 0.428 | 2.132 | 2.279 |
| HC | Y104F | 1.527 | 1.463 | 0.410 | 0.527 | 0.638 | 0.574 | 0.183 | 0.241 | 2.471 | 2.217 |
| HC | Y104L | 1.343 | 1.585 | 0.418 | 0.557 | 0.681 | 0.646 | 0.204 | 0.247 | 2.213 | 2.152 |
| HC | A107E | 1.556 | 1.487 | 0.505 | 0.500 | 0.665 | 0.648 | 0.203 | 0.232 | 2.318 | 2.324 |
| HC | Y108I | 1.148 | 1.257 | 0.282 | 0.411 | 0.533 | 0.492 | 0.173 | 0.179 | 2.353 | 1.964 |
| HC | Y108L | 0.880 | 0.950 | 0.287 | 0.244 | 0.481 | 0.473 | 0.133 | 0.195 | 1.918 | 1.704 |
| HC | Y108W | 1.331 | 1.377 | 0.414 | 0.426 | 0.673 | 0.667 | 0.188 | 0.286 | 2.022 | 1.844 |
| HC | Y108T | 1.494 | 1.566 | 0.292 | 0.528 | 0.608 | 0.709 | 0.192 | 0.220 | 2.333 | 1.961 |
| HC | Y108S | 1.293 | 1.358 | 0.311 | 0.404 | 0.595 | 0.565 | 0.169 | 0.174 | 2.288 | 2.079 |
| HC | Y108N | 1.409 | 1.374 | 0.360 | 0.435 | 0.623 | 0.640 | 0.182 | 0.193 | 2.205 | 2.114 |
| HC | Y108K | 0.183 | 0.541 | 0.149 | 0.164 | 0.266 | 0.261 | 0.109 | 0.105 | 1.380 | 1.471 |
| HC | W109M | 0.583 | 0.678 | 0.175 | 0.205 | 0.303 | 0.314 | 0.108 | 0.128 | 2.042 | 1.616 |
| HC | W109I | 0.440 | 0.466 | 0.143 | 0.151 | 0.241 | 0.234 | 0.099 | 0.108 | 1.907 | 1.420 |
| HC | G110A | 1.906 | 2.205 | 0.608 | 0.910 | 1.130 | 1.104 | 0.360 | 0.334 | 1.843 | 2.205 |
| HC | G110D | 1.639 | 1.450 | 0.431 | 0.445 | 0.731 | 0.713 | 0.218 | 0.191 | 2.138 | 2.156 |
| HC | G110T | 0.770 | 0.783 | 0.203 | 0.176 | 0.381 | 0.399 | 0.145 | 0.112 | 1.992 | 1.482 |
| HC | G112A | 1.162 | 1.194 | 0.303 | 0.392 | 0.623 | 0.644 | 0.180 | 0.147 | 1.860 | 2.173 |
| LC | I033M | 2.183 | 2.207 | 0.721 | 0.639 | 1.336 | 1.183 | 0.372 | 0.348 | 1.750 | 1.889 |
| LC | I033L | 2.051 | 2.115 | 0.639 | 0.797 | 1.071 | 1.047 | 0.287 | 0.315 | 1.968 | 2.379 |
| LC | I033T | 0.928 | 0.912 | 0.244 | 0.237 | 0.389 | 0.388 | 0.134 | 0.128 | 2.367 | 1.838 |
| LC | I033F | 0.780 | 0.765 | 0.193 | 0.215 | 0.346 | 0.346 | 0.124 | 0.124 | 2.235 | 1.646 |
| LC | I033A | 0.922 | 1.005 | 0.274 | 0.265 | 0.462 | 0.486 | 0.149 | 0.153 | 2.031 | 1.785 |
| LC | I033C | 1.370 | 1.356 | 0.399 | 0.417 | 0.642 | 0.708 | 0.196 | 0.233 | 2.025 | 1.912 |
| LC | I033V | 2.386 | 2.389 | 1.302 | 1.776 | 1.895 | 1.804 | 0.703 | 0.766 | 1.292 | 2.085 |
| LC | Y086H | 0.503 | 0.561 | 0.140 | 0.165 | 0.232 | 0.275 | 0.103 | 0.103 | 2.107 | 1.481 |
| LC | Y086F | 0.496 | 0.517 | 0.157 | 0.158 | 0.241 | 0.253 | 0.099 | 0.097 | 2.051 | 1.602 |
| LC | F098S | 0.704 | 0.726 | 0.165 | 0.225 | 0.288 | 0.303 | 0.111 | 0.148 | 2.422 | 1.503 |
| LC | F098Y | 2.236 | 2.283 | 0.899 | 1.225 | 1.360 | 1.352 | 0.399 | 0.000 | 1.667 | 2.291 |
| LC | F098M | 0.408 | 0.452 | 0.136 | 0.144 | 0.190 | 0.185 | 0.096 | 0.096 | 2.296 | 1.457 |
| LC | G099D | 0.734 | 0.717 | 0.200 | 0.214 | 0.330 | 0.285 | 0.111 | 0.123 | 2.373 | 1.774 |
| LC | G099S | 1.948 | 1.945 | 0.684 | 0.732 | 1.140 | 1.225 | 0.314 | 0.321 | 1.648 | 2.230 |
| LC | G099L | 0.546 | 0.501 | 0.160 | 0.188 | 0.262 | 0.256 | 0.110 | 0.101 | 2.020 | 1.660 |
| LC | G099T | 0.845 | 0.809 | 0.233 | 0.227 | 0.432 | 0.415 | 0.137 | 0.127 | 1.951 | 1.742 |
| LC | G099H | 0.448 | 0.445 | 0.139 | 0.152 | 0.253 | 0.251 | 0.098 | 0.092 | 1.771 | 1.528 |

^{a}HC = Heavy Chain, LC = Light Chain

### Example 6

### Generation and Screening of a Combinatorial Library

A combinatorial library of anti-EGFR variant members is generated that contains antibody members having all combinations of mutations LC-I29S, HC-V24E, HC-S25C, HC-F27R, HC-R97H and/or HC-Q111P, together with HC-Y104E. The multiple point mutants are generated in the heavy chain variable region of the HC-Y104E Cetuximab mutant anti-EGFR antibody described in Example 1 (containing a variable heavy chain set forth in SEQ ID NO: 74 or a variable light chain set forth in SEQ ID NO: 8) or in the heavy chain variable region of the humanized form of HC-Y104E generated in Example 4 by site-directed mutagenesis (containing a variable heavy chain set forth in SEQ ID NO: 61 and a variable light chain set forth in SEQ ID NO: 183). Each member of the library is sequenced and stored as glycerol stocks at −80° C. Among the combination mutants generated are mutants as set forth in Table 35.

TABLE 35
Exemplary Y104E combination modified anti-EGFR antibodies

SEQ ID NO

| Mutation(s) | HC | LC |
|---|---|---|
| HC-S25C/HC-Y104E | 79 | 8 |
| HC-S25V/HC-Y104E | 88 | 8 |
| HC-S25V/HC-Y104E/HC-Q111P | 91 | 8 |
| HC-S25V/HC-S53G/HC-Y104E | 94 | 8 |
| HC-S25V/HC-S53G/HC-Y104E/HC-Q111P | 97 | 8 |
| HC- F027G/Y104E | 315 | 8 |
| HC- F027G/Y104E/Q111P | 318 | 8 |
| HC- F027G/S053G/Y104E | 321 | 8 |
| HC- F027G/S053G/Y104E/Q111P | 324 | 8 |
| HC-T30F/HC-Y104E | 100 | 8 |
| HC-T30F/HC-Y104E/HC-Q111P | 103 | 8 |
| HC-T30F/HC-S53G/HC-Y104E | 106 | 8 |
| HC-T30F/HC-S53G/HC-Y104E/HC-Q111P | 109 | 8 |
| HC-S53G/HC-D72L/HC-Y104E | 118 | 8 |
| HC-S53G/HC-D72L/HC-Y104E/HC-Q111P | 121 | 8 |
| HC-S53G/HC-Y104E | 82 | 8 |
| HC-S53G/HC-Y104E/HC-Q111P | 85 | 8 |
| HC-D72L/HC-Y104E | 112 | 8 |
| HC-D72L/HC-Y104E/HC-Q111P | 115 | 8 |
| HC-Y104E/HC-Q111P | 76 | 8 |
| HC-Y104E/LC-I29S | 72 | 214 |
| HC-Y104E/HC-Q111P/LC-I29S | 76 | 214 |

For screening, an expression vector encoding a member of the library is separately expressed in CHO cells as IgG antibodies and supernatants collected. The library is screened by pH sensitive ELISA, as described in Example 3. Exemplary antibodies exhibiting higher binding activity at pH 6.0 compared to pH 7.4 are selected.

### Example 7

### Binding Affinity of Identified Hits

Bio-layer interferometry is performed to measure binding of variant anti-EGFR antibodies to EGFR at pH 6.5 and pH 7.4. The dissociation constant (K_{D}) of Cetuximab and Y104E-containing variant anti-EGFR antibodies for sEGFR are measured by bio-layer interferometry in a 96-well format using an Octet QKe instrument (ForteBio, Menlo Park, Calif.). Data Acquisition software is used for all the operation steps including biotinylated sECD EGFR ligand loading and antibody association and dissociation steps. The ligand loading and antibody association and dissociation steps are performed with two groups, one at pH 6.5 and the other at pH 7.4.

Biotinylated sECD EGFR is prepared by adding 15 μL of NHS-PEG4-Biotin solution (20 mM in ultrapure water) (PIERCE, Cat#21329) to 1 mg of sEGFR in solution, and incubating the reaction mixture at room temperature for 30 minutes. Nonreacted NHS-PEG4-Biotin is removed by dialysis, and the protein concentration of biotinylated sEGFR is measured by BCA protein assay (PIERCE, Cat#23225) according to manufacturer's instruction.

1. Binding Affinity of Anti-EGFR Variants at pH 6.5 and pH 7.4

To assess the difference in binding at different pH, biotinylated sECD EGFR is bound to a streptavidin biolayer in PBS at pH 6.5 or at pH 7.4 (ligand loading step). The streptavidin sensors are then dipped in wells containing PBS (pH 6.5 or pH 7.4) for 1 minute. Then, the sensors are dipped in wells containing biotinylated sEGFR (50 μg/mL) in PBS (pH 6.5 or pH 7.4) for 2 minutes. Sensors are rinsed in wells containing PBS (pH 6.5 or pH 7.4). During all steps, the plate is agitated at 1000 rpm.

To measure association rates, the immobilized sECD-EGFR sensors are dipped into wells with antibody (Cetuximab or Y104E variant anti-EGFR antibodies) at 22 nM or 66.7 nM in PBS at pH 6.5 or 7.4 for 2 min. To measure dissociation rates, antibody bound sensors are dipped in PBS wells at pH 6.5 or 7.4 for 4 min. Association and dissociation of sEGFR and antibody (Cetuximab or Y104E variant anti-EGFR antibodies) are quantitated by measuring changes in the interference pattern generated from light reflected from the optical layer and the biolayer. Association rates, dissociation rates and K_{D} values are calculated with Software Data Analysis (v. 6.4) using global curve fitting.

### Example 8

### EGFR Phosphorylation

The concentration of phosphorylated EGFR from human neonatal keratinocytes and A431 cells treated with the reference Cetuximab antibody or Y104E anti-EGFR antibody variants is measured by ELISA (RnD systems reagents, #DYC3570-2).

1. Preparation of Samples

Approximately 10,000 cells, human neonatal keratinocytes (Invitrogen C-001-5C) or A431 cells (ATCC CRL 1555); are plated in wells of a 96 well plate (BD Falcon #35-3072). After overnight incubation at 37° C. in a humidified atmosphere of 5% CO₂ incubator, the cells are washed, resuspended in serum free Dulbecco's Modified Eagle Medium (DMEM) and incubated overnight under the same conditions. The cells are washed with cold Phosphate Buffered Saline (PBS; 137 mM NaCl, 2.7 mM KCl, 8.1 mM Na₂HPO₄, 1.5 mM KH₂PO₄, pH 7.2-7.4). Then, the plates are divided into two groups. In the first group, 10 μg/mL purified Cetuximab or anti-EGFR antibody containing HC-Y104E variant or buffer control is added in phosphate buffer adjusted to pH 7.4. In the second group, purified Cetuximab or anti-EGFR antibody containing HC-Y104E variant or buffer control is added in phosphate buffer adjusted to pH 6.5.

For A431 cells, a dose-response also is performed, whereby Cetuximab or anti-EGFR antibody containing HC-Y104E variant are added to samples at a concentration of 30 μg/mL, 10 μg/mL, 3.33 μg/mL, 1.11 μg/mL, 0.37 μg/mL, 0.123 μg/mL and 0.001 μg/mL. The cells are incubated for 15-30 minutes at 37° C.

After the initial incubation with antibody, EGF (RnD Systems, catalog no. 236-E) (100 μg/mL) is added separately to the cells in the same buffer as the antibody. Control cells also are tested where no antibody is added (EGF only) or where no antibody or EGF is added (no Rx). The cells are incubated for 15-30 minutes at 37° C. After incubation with the antigen, the cells are washed with cold PBS, and cold lysis buffer (1% NP-40 Alternative, 20 mM Tris (pH 8.0), 137 mM NaCl, 10% glycerol, 2 mM EDTA, 1 mM activated sodium orthovanadate, 10 μg/mL Aprotinin, 10 μg/mL Leupeptin) is added. The lysate is collected and assayed immediately or stored at ≦−70° C. for subsequent analysis.

2. ELISA

A 96 well Microplate (Costar #2592) is coated with rat anti-human anti-phospho EGFR capture antibody (8.0 μg/mL in PBS, 100 μL/well) (R&D Systems #842428) overnight at room temperature. Each well is aspirated, and washed with wash buffer (0.05% TWEEN® 20 in PBS, pH 7.2-7.4 (R&D Systems # WA126) for a total of five washes. Plates are blocked for 1-2 hours at room temperature with 300 μL of Block Buffer (1% BSA, 0.05% NaN₃ in PBS, pH 7.2-7.4). The wells are aspirated and washed with wash buffer for a total of five washes. Cell lysate (Cetuximab, anti-EGFR antibody Y104E variant antibody, EGF only or No Rx) is diluted in IC Diluent (1% NP-40 Alternative, 20 mM Tris (pH 8.0), 137 mM NaCl, 10% glycerol, 2 mM EDTA, 1 mM activated sodium orthovanadate) and 100 μL are added. The aspiration and wash steps are repeated, and 100 μL mouse anti-human phospho-EGF R (Y1068) antibody conjugated to HRP (20 mM Tris (pH 8.0), 137 mM NaCl, 0.05% TWEEN® 20, 0.1% BSA) is added. Plates are sealed and incubated for 2 hours at room temperature. The aspiration and wash steps are repeated. Substrate (1:1 mixture of H₂O₂ and Tetramethylbenzidine (R&D Systems, Catalog # DY999)) (100 μL) is added to each well and the plate is incubated for 20 minutes at room temperature. Stop solution (2 N H₂SO₄ (R&D Systems, Catalog # DY994) (50 μL) is added, and the optical density (OD) of the wells is measured immediately in a microplate reader set to 450 nm with a wavelength correction at 540 nm or 570 nm

### Example 9

### Growth of Human and Neonatal Keratinocytes in the Presence of Cetuximab or HC-Y104E Anti-EGFR Antibody

The growth of Human neonatal keratinocytes (Invitrogen C-001-5C) and Human adult keratinocytes (Invitrogen C-005-5C) is measured after incubation with Cetuximab or anti-EGFR antibody containing HC-Y104E variant. The antibody to be tested is added to normal growth medium (10% FBS, DMEM (pH 7.4)) to a concentration of 10 μg/mL, 3.33 μg/mL, 1.11 μg/mL, 0.37 μg/mL, 0.123 μg/mL, 0.0411 μg/mL, 0.0137 μg/mL and 0.00457 μg/mL.

Human neonatal keratinocytes and human adult keratinocytes are added (1000 cells/well) to a 96-well plate (BD Falcon 35-3072) in the presence of the normal growth medium containing Cetuximab or anti-EGFR antibody containing HC-Y104E variant. Each condition is assayed in 5 replicate wells (i.e., n=5 per condition). Cells are incubated for 5 days at 37° C. in a humidified atmosphere of 5% CO₂ incubator. Cell growth is measured by CellTiter-Glo® Luminescent Cell Viability Assay (Promega Cat# G-7571) and expressed as percent surviving cells compared to control cells grown without antibody.

### Example 10

### Effects of Cetuximab or Anti-EGFR Antibody Containing HC-Y104E Variant on Tumor Growth in Xenograft Models

A431 epidermoid carcinoma cells, FaDu hypopharyngeal carcinoma cells, and engineered cell lines A431LDHA and A431CA9, derived from A431 cells, are used to generate xenograft tumor models, which are used to evaluate the antitumor activity of Cetuximab and an anti-EGFR antibody containing HC-Y104E variant.

1. Subcutaneous A431 Tumors

Male, athymic NCr-nu/nu mice are inoculated with a 0.1 mL subcutaneous (SC) injection of 3.3×10⁶ A431 epidermoid carcinoma cells (ATCC CRL1555) suspended in RPMI-1640 medium into their right flanks. When the tumors reach a size of ˜100 mm³, animals are randomized into five study groups (n=4/group): (1) Group 1—vehicle (Cetuximab buffer), (2) Group 2—0.1 mg/mouse (4 mg/kg body weight) Cetuximab, (3) Group 3—1.0 mg/mouse (40 mg/kg body weight) Cetuximab, (4) Group 4—0.1 mg/mouse (4 mg/kg body weight) HC-Y104E variant anti-EGFR antibody, or (5) Group 5-1.0 mg/mouse (40 mg/kg body weight) HC-Y104E variant anti-EGFR antibody. Mice are intraperitoneally (IP) administered 0.1 mg or 1.0 mg of Cetuximab reference or HC-Y104E variant anti-EGFR antibody variant or vehicle control twice weekly on days 0, 4, 7, 11, and 14. Tumor growth is measured as described above on days 0, 4, 7, 11, 14 and 18, except that animals treated with only vehicle are sacrificed on day 14.

Tumor Growth Inhibition (TGI) for the Cetuximab or HC-Y104E variant anti-EGFR antibody treatment groups is calculated using the formula: % TGI=[1−(T_{B}−T_{A})/(C_{B}−C_{A})]×100; where T_{B} is the average tumor volume (mm³) in the treatment group at 14 days after initiation of treatment, T_{A} is the average tumor volume (mm³) in the treatment group at day 0 before treatment, C_{B} is the average tumor volume in the control group at 14 days after initiation of treatment, and C_{A} is the average tumor volume in the control group at day 0 before treatment (see, e.g., Teicher B A and Andrews P A: Anticancer Drug Development, Guide: Preclinical Screening, Clinical Trials and Approval, 2^{nd} edition. Humana Press, Totowa, N.J., pp. 134, 2004 and T. Friess et al., (2006) Anticancer Research 26:3505-3512). TGI is then compared across the wild-type Cetuximab and HC-Y104E variant anti-EGFR antibodies.

2. Subcutaneous FaDu Tumors:

Male, athymic NCr-nu/nu mice are inoculated with a 0.1 mL subcutaneous (SC) injection of 5.0×10⁶ FaDu hypopharyngeal carcinoma cells (ATCC) suspended in RPMI-1640 medium into their right flanks. When the tumors reach a size of ˜100 mm³, animals are randomized into five study groups (n=4/group): (1) Group 1—vehicle (Cetuximab buffer), (2) Group 2—4 mg/kg Cetuximab, (3) Group 3—40 mg/kg Cetuximab, (4) Group 4—4 mg/kg HC-Y104E variant anti-EGFR antibody, or (5) Group 5—40 mg/kg HC-Y104E variant anti-EGFR antibody. The antibodies or vehicle only are administered intraperitoneally (IP) twice weekly on days 0, 3, 7, and 10. Tumor growth is measured using calipers as described above on days −1, 3, 7, 10, 14 just prior to antibody administration. Tumor growth inhibition is then compared across the antibodies evaluated

### Example 11

### EGFR Expression in Subcutaneous Tumors and in Primate Skin

Detection of EGFR in subcutaneous xenograft tumors and in harvested primate skin was confirmed and assessed by immunohistochemistry (IHC).

a. EGFR Expression in Subcutaneous Tumors

Immunohistochemistry (IHC) was used to assess the levels of EGFR expression in A431 human tumors grown as xenografts in nude mice as described in Example 10.1. A431 subcutaneous tumors were harvested and fixed in 10% neutral buffered formalin (NBF) and embedded in paraffin. Five (5) μm sections were mounted on slides and dried. Prior to staining, the slides were deparaffinized and rehydrated. Sections were immunolabeled using an EGFR IHC kit (Dako, Carpinteria, Calif.). Staining was visualized with 3,3′-diaminobenzidine (DAB) according to the manufacturer's instructions. Nuclei were counterstained with hematoxylin. Micrographs were captured with a Nikon Eclipse TE2000U microscope coupled to a Insight FireWire digital camera (Diagnostic Instruments, Michigan). The intense cell membrane positivity for EGFR in the tumor cells confirmed that xenograft tumors derived from A431 cells retain high levels of EGFR expression.

b. EGFR Expression in Primate Skin

Immunohistochemistry (IHC) was used to assess the levels of EGFR expression in human and non-human primate skin samples. Human skin samples were obtained from a local surgical center; cynomolgus monkey, marmoset monkey and squirrel monkey skin were received from Worldwide Primates Inc.(Miami, Fla.). Formaldehyde fixed samples of human, cynomolgus monkey, marmoset monkey and squirrel monkey were sectioned and processed for IHC with the EGFR IHC kit (DAKO) as described above. Nuclei were counterstained with hematoxylin. Micrographs were captured above, with 20× and 40× objectives.

As expected, the membranes of the basal keratinocytes exhibited intense staining for EGFR. The basal keratinocytes of cynomolgus and squirrel monkey skin tissues also exhibited staining, with the cynomolgus skin staining with slightly less intensity than that observed for human and squirrel monkey tissues. The marmoset monkey skin did not exhibit any detectable staining, even when using a 40× objective. These results indicate that cynomolgus monkey and squirrel monkey EGFR, but not Marmoset monkey EGFR, are sufficiently similar to human EGFR to be recognized by the anti-human EGFR monoclonal antibody.

### Example 12

### Binding of an Anti-EGFR Antibody Containing HC-Y104E Variant to A431 Subcutaneous Tumors or Skin Grafts Ex Vivo

HC-Y104E mutant antibody binding to subcutaneous tumors and primate skin grafts in vivo are evaluated, since EGFR is expressed in subcutaneous tumors and primate skin as described in the previous Example.

1. Ex Vivo Binding Studies to Subcutaneous Tumors

Immunofluorescence (IF) is used to assess and compare the abilities of Cetuximab and HC-Y104E variant anti-EGFR antibodies to bind human EGFR. Cetuximab and HC-Y104E Anti-EGFR antibodies are conjugated to DyLight⁵⁹⁴ at 10, 5, 1, 0.3, 0.1 μg/mL in PBS using the DyLight 594 Antibody Labeling Kit (Thermo Scientific; Rockford, Ill.), according to the manufacturer's instructions. Following sectioning, frozen sections of A431 tumors are fixed for 10 min in cold acetone and incubated for one hour with 5 mg/mL or 1 μg/mL of either DyLight⁵⁹⁴-conjugated Cetuximab or HC-Y104E antibody. After washing in PBS, sections are counter-stained with DAPI (4′,6-Diamidino-2-Phenylindole, Dihydrochloride) (Molecular Probes, Eugene). Micrographs are captured, with 20× and 40× objectives, using a Nikon Eclipse TE2000U microscope coupled to a Insight FireWire digital camera (Diagnostic Instruments, Michigan) using the same settings for each image to allow for comparison between experimental conditions. EGFR binding is then compared across antibodies.

2. Ex Vivo Binding Studies to Primate Skin

Immunofluorescence (IF) is used to assess the ability of Cetuximab and HC-Y104E variant antibodies to bind human EGFR in various primate skin samples. Cryosections of human, cynomolgus monkey, marmoset monkey and squirrel monkey (human skin received from a local surgical center; cynomolgus monkey, marmoset monkey and squirrel monkey skin received from Worldwide Primate, Florida), are directly immunolabeled at neutral pH as described above, using 1.0 μg/mL Cetuximab or HC-Y104E variant conjugated to Alexafluor 594 (Thermo Scientific DyLight 594 Antibody Labeling Kit; Rockford, Ill.). Nuclei are counterstained with DAPI. Micrographs are captured as described above, using 20× and 40× objectives Immunolabeling of pre-keratinocytes and basal cells is then visually assessed and compared across antibodies.

### Example 13

### Selective Binding of Fluorescently-Labeled HC-Y104E Mutant Anti-EGFR Antibodies to Tumors Versus Skin In Vivo

Cetuximab, HC-Y104E variant anti-EGFR antibodies, and a control Human IgG are labeled at room temperature for 60 minutes with DyLight755 Sulfydryl-Reactive Dye (DL755) (Thermo Scientific, Rockford, Ill.), a near-IR fluor. The binding of DL755 labeled IgG, Cetuximab, and HC-Y104E mutant antibodies to xenograft tumors or human or monkey skin grafts is assessed using the IVIS Caliper fluorescent imaging system with an excitation wavelength of 745 nm and an emission wavelength of 800 nm Images are captured before administration of antibody and at 1 minute, 2 minutes, 10 minutes, 60 minutes, 120 minutes, 240 minutes, 360 minutes, 1 day, and daily after administration of the antibodies. In the human skin graft models, images also are captured at 10 days post administration of the antibodies.

1. Cetuximab and HC-Y104E Binding to Subcutaneous A431 Tumors

a. Whole Animal Imaging

A431 xenograft tumors are produced by injecting A431 cells into the right flanks of nude mice as described in Example 10 above. Twenty-one (21) days post-implantation, the mice are administered 10 μg/mouse (0.5 mg/kg) Human IgG^{DL755}, HC-Y104E variant^{DL755}, or Ceuximab^{DL755}. The DL755 label is detected, in 2-4 animals/group, using the IVIS Caliper fluorescent imaging system with an excitation wavelength of 745 nm and an emission wavelength of 800 nm Images are captured before administration of antibody and at 4 hr after administration of the antibodies, and then daily for 7 days. The binding demonstrated by the HC-Y104E mutant antibodies are then compared to that of Cetuximab^{DL755} and the negative control antibody, human IgG^{DL755}.

b. Whole Animal Imaging and Immunohistochemistry

As described above, nude mice are injected with A431 cells in the right flanks to generate A431 tumors. On day 21 post-A431 cell implantation, the mice are administered a single i.v. dose of IgG^{DL755}, HC-Y104E variant^{DL755}, or Cetuximab^{DL755} at 1 mg/mouse in at least 2 mice per group. 48 hr after the dose of antibody, the tumors are visualized using the IVIS Caliper fluorescent imaging system as described above.

Following tumor imaging, immunohistochemical staining of human IgG^{DL755}, HC-Y104E variant^{DL755}, and Ceuximab^{DL755}-injected mice, by F_{c} detection is performed to assess the localization of antibody binding in more detail. After imaging, the mice are perfused, the tumors are harvested, and cryosections of the tumor are incubated with HRP-conjugated goat anti-human IgG secondary antibody for detection of the F_{c} regions of the injected antibodies using standard immunohistochemical staining methods, using DAB as the HRP substrate to enable visualization. The stained tissues are examined using a Nikon Eclipse TE2000U microscope coupled to an Insight FireWire digital camera (Diagnostic Instruments, Michigan) equipped with a 20× objective.

2. Cetuximab and HC-Y104E Binding to Subcutaneous PC-3 Tumors

Xenograft tumors derived from PC-3 cells are generated by injecting 2×10⁶ PC-3 cells (Caliper Life Sciences), in 100 μl serum-free Opti-MEM®, into the right peritibial muscle of male nude mice. 35 days after implantation, the PC-3 tumor bearing mice are administered 10 μg/mouse (0.5 mg/kg) human IgG^{DL755}, HC-Y104E variant^{DL755}, or Ceuximab^{DL755} (n=at least 2/group), and the DL755 label is detected using the IVIS Caliper fluorescent imaging system as described above. Images are captured before administration of antibody and then daily for 5 days. A parallel immunohistochemical study also is performed on the PC-3 xenograft tumors, 48 hours following i.v. administrations of human IgG (control), HC-Y104D, or Cetuximab (1 mg/mouse) by F_{c} detection as described above.

3. Binding of Cetuximab and HC-Y104E Anti-EGFR Antibodies to Human Skin Grafts

As a model to assess skin toxicity, binding of HC-Y104E variant anti-EGFR antibodies to human and monkey skin grafts implanted in mice is assessed in vivo. HC-Y104E variant anti-EGFR antibodies, Cetuximab and Human IgG, as positive and negative controls, respectively, are labeled at room temperature for 60 minutes with DyLight755 Sulfydryl-Reactive Dye (DL755) (Thermo Scientific, Rockford, Ill.), a near-IR fluor. Human split thickness skin graft (STSG) (human skin received from a local surgical center) and human foreskin grafts (purchased from NDRI (1628 JFK Blvd, 8 Penn Center, Philadelphia, Pa.) are surgically transplanted on the left dorsal flank in Ncr nu/nu mice. EGFR expression is confirmed in the human skin grafts on days 70 and 32 post implantation, respectively, by anti-EGFR IHC kit (Dako). On day 32 and day 36 post-implantation, the labeled antibodies are administered by i.v. to mice with human skin grafts at a dose of 300 μg/mouse. Images of DL755 signal are taken on each of days 1-10 post administration, and visual assessment is used to evaluate and compare circulating systemic signal and localization of signal to the skin graft locations.

In another study, on day 21 post implantation, mice receiving human foreskin grafts are analyzed for antibody binding, using the same method (n=3/group), before and 4 hr after intravenous administration of 10 μg/mouse (0.5 mg/kg) human IgG^{DL755}, HC-Y104E variant^{DL755}, or Cetuximab^{DL755} and then daily thereafter for a total of 6 days.

On day 28 post-implantation, the binding the antibodies to the human foreskin skin grafts is assessed by immunohistochemistry using standard methods. Mice receiving human foreskin grafts are administered a single i.v. dose of IgG, Y104E variant, or Cetuximab at 1 mg/mouse. 48 hr after the dose of antibody, the mice are perfused and cryosections are incubated with HRP-conjugated goat anti-human IgG secondary antibody. DAB is used as the HRP substrate. The stained tissues are examined using a Nikon Eclipse TE2000U microscope coupled to a Insight FireWire digital camera (Diagnostic Instruments, Michigan) equipped with a 40× objective.

4. Binding of Cetuximab and HC-Y104E Anti-EGFR Antibodies to Monkey Skin Grafts

Monkey STSG (cynomolgus monkey skin received from BioTox) are surgically transplanted on the left dorsal flank in 7 Ncr nu/nu mice. EGFR expression is confirmed in the monkey skin grafts on days 70 and 32 post implantation, respectively, by anti-EGFR IHC kit (Dako). On day 32 and day 36 post-implantation, the DL755-labeled antibodies, described above, are administered by i.v. to mice with monkey skin graft models at a dose of and 30 μg/mouse. Circulating and localized fluorescence signal is detected and images captured as described above on each of 1-9 days post antibody administration.

5. A431 Tumor Vs. Skin Binding

Quantified fluorescent signal intensities are used to determine the ratio of tumor:skin binding for Cetuximab and HC-Y104E mutant antibodies by dividing the DL755 signal intensity of the tumor binding, determined in part 1 of this Example, by the corresponding DL755 signal intensity of the human skin graft binding from the same antibody determined in part 2 of this Example (n=at least 2/group). The ratios are then normalized to the tumor:skin binding ratio calculated for the control IgG-administered animals.

### Example 14

### Effects of Cetuximab on Skin Toxicity in a Skin Graft Model

Donor skin from the palpebral fissure of a patient is harvested and split thickness skin grafts are transplanted to 4 Ncr nu/nu mice. Starting on day 15 post skin transplantation, two of the mice are each intravenously administered 2 mg HC-Y104E antibody variant or Cetuximab (100 mg/kg, HED 60 mg/kg) twice weekly for 4 weeks. On day 35 post-antibody administration (i.e., 7 days after the final dose of antibody) the condition of the skin grafts is visually assessed, for example, for shrinking of the graft and/or local irritation (e.g., redness, dryness). Samples of donor skin and grafted skin, containing both the human donor skin graft and the adjacent skin of the host mouse, are then collected and analyzed by immunohistochemistry using the anti-EGFR IHC kit (Dako). Staining in the pre-karatinocytes and basal cells in the human skin graft is compared to the staining in the pre-karatinocytes and basal cells of the adjacent skin native to the host mouse.

### Example 15

### Cetuximab and Chemotherapy Combinatorial Treatment

The efficacy of Cetuximab in combination with the chemotherapeutic reagent, cisplatin, was evaluated for the inhibition of A431 xenograft tumor growth.

Subcutaneous A431 xenograft tumors were established in male nude mice as described in Example 10 above. When the tumors were approximately 100-200 mm³ in size, animals were randomized into nine study groups (n=5/group), as set forth in Table 36, and administered Cetuximab by intraperitoneal administration twice per week and/or cisplatin twice per week by intravenous administration. Specifically, the test article(s) were administered on days 0, 4, 7, 11, and 14.

TABLE 36
Cetuximab and/or Cisplatin Dose and Resulting Tumor Growth Inhibition

| | | Cetuximab Dose | | Cisplatin Dose | | % Tumor Growth |
|---|---|---|---|---|---|---|
| Group | Test Article(s) | mg/mouse | mg/kg | mg/mouse | mg/kg | Inhibition |
| 1 | Vehicle | — | — | — | — | — |
| 2 | Cetuximab | 0.1 | 4 | — | — | 41.8 |
| 3 | Cetuximab | 0.3 | 12 | — | — | 65.9* |
| 4 | Cisplatin | — | — | 0.04 | 1.5 | 16.7 |
| 5 | Cisplatin | — | — | 0.125 | 5 | 34.2* |
| 6 | Cetuximab + Cisplatin | 0.1 | 4 | 0.04 | 1.5 | 53.2* |
| 7 | Cetuximab + Cisplatin | 0.1 | 4 | 0.125 | 5 | 50.2* |
| 8 | Cetuximab + Cisplatin | 0.3 | 12 | 0.04 | 1.5 | 74.1* |
| 9 | Cetuximab + Cisplatin | 0.3 | 12 | 0.125 | 5 | 85.2* |

*= p < 0.05 vs. vehicle only

Tumor growth, measured as tumor volume (mm³), was determined on days 1, 4, 7, 11 and 14 using digital calipers and calculation as described in Example 10. Tumor growth inhibition (TGI) for the treatment groups was calculated using the formula: % TGI=[1−(T_{B}−T_{A})/(C_{B}−C_{A})]×100; where T_{B} is the average tumor volume (mm³) in the treatment group at day 14, T_{A} is the average tumor volume (mm³) in the treatment group the day before the first treatment (day −1), C_{B} is the average tumor volume in the vehicle only control group at day 14, and C_{A} is the average tumor volume in the vehicle only control group the day before the first treatment (day −1) (see, e.g., Teicher B A and Andrews P A: Anticancer Drug Development, Guide: Preclinical Screening, Clinical Trials and Approval, 2^{nd} edition. Humana Press, Totowa, N.J., pp. 134, 2004 and T. Friess et al., (2006) Anticancer Research 26:3505-3512). The results are set forth in Table 36 above.

While 1.5 mg/kg cisplatin did not significantly inhibit tumor growth on its own, it did contribute to additional tumor growth inhibition when in combination with Cetuximab at 4 mg/kg (41.8% TGI for Cetuximab alone vs. 53.2% TGI for the combination) and at 12 mg/kg (65.9% TGI for Cetuximab alone vs. 74.1% TGI for the combination). Treatment with 5 mg/kg cisplatin alone resulted in 34.2% TGI and further contributed to TGI when in combination with Cetuximab at 4 mg/kg (41.8% TGI for Cetuximab alone vs. 50.2% TGI for the combination) and at 12 mg/kg (65.9% TGI for Cetuximab alone vs. 85.2% TGI for the combination). The maximum tumor growth inhibition was observed with 12 mg/kg Cetuximab+5 mg/kg cisplatin.

### Example 16

### Cetuximab Vs. HC-Y104E Variants and Chemotherapy Combinatorial Treatment

The efficacy of Cetuximab versus HC-Y104E anti-EGFR variants in combination with the chemotherapeutic reagent cisplatin, are evaluated for the inhibition of A431 xenograft tumor growth.

Subcutaneous A431 xenograft tumors are established in male nude mice as described above. When the tumors are approximately 100 mm³ in size, animals are randomized into study groups (n=5/group), and administered Cetuximab or HC-Y104E-containing variant (Y104E), by IP administration, and/or cisplatin, by IV administration, twice per week, as indicated in Table 37 below. Specifically, the test article(s) are administered on days 0, 4, 7 and 11. Tumor volume (mm³) is determined on days −1, 4, 7, 11 and 14 as described previously.

TABLE 37
Cetuximab, HC-Y104D and/or Cisplatin Dose

| | | Cetuximab Dose | | HC-Y104E Dose | | Cisplatin Dose | |
|---|---|---|---|---|---|---|---|
| Group | Test Article(s) | mg/mouse | mg/kg | mg/mouse | mg/kg | mg/mouse | mg/kg |
| 1 | Vehicle | — | — | — | — | — | — |
| 2 | Cetuximab | 0.3 | 12 | — | — | — | — |
| 3 | Cisplatin | — | — | — | — | 0.125 | 5 |
| 4 | Y104E | — | — | 0.3 | 12 | — | — |
| 5 | Y104E | — | — | 1.0 | 40 | — | — |
| 6 | Cetuximab + Cisplatin | 0.3 | 12 | — | — | 0.125 | 5 |
| 7 | Y104E + Cisplatin | — | — | 0.3 | 12 | 0.125 | 5 |
| 8 | Y104E + Cisplatin | — | — | 1.0 | 40 | 0.125 | 5 |

### Example 17

### Effect of Anti-EGFR Antibody-Drug Conjugates (ADCs) on Tumor Cell and Keratinocyte Cell Growth Inhibition

Anti-EGFR antibody-drug conjugates (ADCs) are generated by fusing the immunotoxin Saporin to Cetuximab or HC-Y104E variant anti-EGFR antibody by either mixing biotinylated antibodies and streptavidin-Saporin (Advanced Targeting Systems Bio, Cat# IT-27) or using a cleavable protein cross-linker (service provided by Advanced Targeting Systems Bio) to permit drug release inside the target cells.

For biotin-streptavidin based ADC formation, antibodies at a concentration of 1-2 mg/ml in 0.1 M phosphate buffer, pH 7.2 are oxidized, converting adjacent hydroxyl groups of the antibody sugar chains into aldehyde groups, using sodium periodate (NaIO₄) at a final concentration of 5 mg/ml, 4° C. for 30 min. The oxidized antibodies are then dialyzed against 0.1 M phosphate buffer, pH 7.2, and the dialyzed antibodies are mixed with 50 mM hydrazide-biotin prepared in DMSO at volume ratio 9 to 1, resulting in 5 mM hydrazide-biotin in the reaction. The mixture is incubated at room temperature for 2 hours to form hydrazone bonds between the aldehyde groups of the antibodies and hydrazide groups. The biotinylated antibodies are then dialyzed against 1×PBS, and then mixed with streptavidin-saporin in an equal molar ratio to form the antibody-saporin complex. The ADCs are then tested for their abilities to inhibit cell growth of human tumor cell lines, A431 and MDA-MB-468, and a human keratinocyte cell line, HEK-N.

1. Saporin ADC Inhibition of A431 Cell Growth

A431 cells are cultured in RPMI 1640 medium supplemented with 10% fetal bovine serum (FBS; Mediatech). The day before ADC treatment, A431 cells are seeded at 1,000 cells/well in 200 μL volume in clear bottom white 96-well plates. The cells are left untreated or are treated with the Saporin conjugated Cetuximab (Wt-Sap), Saporin conjugated HC-Y104E variant (Y104E-Sap), or Saporin-conjugated human IgG at increasing concentrations starting from 1 μg/mL. The cells are subjected to ADC treatment for 5 days. Live cells are measured on day 5 using the Cell Titer-glo Luminescent kit (Promega) according to the manufacturer's instructions. The percentages of surviving cells are calculated relative to untreated cells and EC₅₀ values are computed using GraphPad Prism.

2. Saporin ADC Inhibition of Neonatal Keratinocyte (HEK-N) Cells

Neonatal Keratinocyte (HEK-N) cells are cultured in growth factor supplemented Epilife medium (Gibco). The day prior to Saporin ADC treatment, HEK-N cells are seeded at 1,000 cells/well in 200 μL volume in clear bottom white 96-well plates. The cells are left untreated or are treated with the Saporin conjugated Cetuximab (Wt-Sap), Saporin conjugated HC-Y104E variant (Y104E-Sap), or Saporin-conjugated human IgG at increasing concentrations starting from a concentration of 1 μg/mL. The cells are subjected to ADC treatment for 5 days. Live cells are measured on day 5 using the Cell Titer-glo Luminescent kit (Promega) according to the manufacturer's instructions. The percentages of surviving cells are calculated relative to untreated cells and EC₅₀ values were computed using GraphPad Prism.

### Example 18

### Effect of Y104D-DM1, Y104D-MMAE and Y104D-MMAF Antibody-Drug Conjugates (ADCs) on A431 Subcutaneous Tumor Growth In Vivo

An A431 tumor xenograft model, which is a model of EGFR+ tumors, was used to assess the tumor growth inhibition activity of antibody drug conjugates (ADCs) of HC-Y104D variant anti-EGFR antibody. In particular, HC-Y104D variant anti-EGFR antibody conjugates containing the cytotoxic drugs maytansinoid DM1, monomethyl auristatin E (MMAE), or monomethyl auristatin F (MMAF) were generated and each assessed in this model. DM1 and MMAE have a comparable potency of 10 pM to 1 nM, and MMAF has a potency of 10 nM to 100 nM (“Second-Generation ADCs.” Biological Drug Products: Development and Strategies. Ed. Wei Wang, Ed. Manmohan Singh. ISBN: 978-1-118-14889-1, 2013. Section 9.4.2.3).

### 1. HC-Y104D Variant Anti-EGFR Antibody Conjugated to Maytansinoid DM1 (Y104D-DM1)

a. Generation of Y104D-MCC-DM1 (Y104D-DM1)

HC-Y104D antibodies were conjugated to the maytansinoid, DM1, via a non-cleavable succinimidyl-4-(N-maleimidomethyl)cyclohexane-1-carboxylate (SMCC) linker. To generate Y104D-DM1 ADCs, 2.5 mL of Y104D, generated as described in Example 2, was loaded onto a PD-10 desalting column (GE Health) that was pre-equilibrated with Conjugation Buffer 1 (50 mM Potassium Phosphate/50 mM Sodium Chloride, pH 6.5, 2 mM EDTA). The sample was followed with 0.5 mL Conjugation Buffer 1, and the de-salted antibody was collected. The concentration of the antibody was measured and adjusted to 20 mg/mL.

A Y104D-MCC conjugate was first generated by adding 10 mM SMCC in Dimethylacetamide (DMA) to the antibody solution at a 7:1 molar ratio (0.933 mL of 10 mM SMCC to 10 mL of 20 mg/mL antibody solution). DMA was then added to the mixture to achieve 10% v/v DMA. The mixture was then incubated at room temperature for 2 hr. The Y104D-MCC conjugate was loaded onto pre-equilibrated PD-10 desalting column. The sample was followed with 0.5 mL Conjugation Buffer 1, and the de-salted antibody was collected. The concentration of the conjugate was calculated using the A₂₈₀ of the eluate (ε=1.45 mg⁻¹ cm⁻¹, or 217500 M⁻¹ cm⁻¹).

The average number of SMCC molecules per antibody was determined by indirect measurement, by determining the reduction of free thiol groups in the solution. 5 μL buffer or Y104D-MCC were added to 5 μL 1 mM cysteine in a microcentrifuge tube, followed by 1 mM Ellman's reagent (5,5′-dithiobis-(2-nitrobenzoic acid) or DTNB) in PBS (diluted from 0.1 M in DMSO stock). After vortexing, the mixtures were incubated at room temperature for 5 min. The absorbance was measured at 280 nm and 412 nm, and the concentration was calculated using the A280 and the extinction coefficient, ε=210,863 cm⁻¹M⁻¹. The cysteine concentration in the buffer only sample was determined using the extinction coefficient of the chromophore, 5-mercapto-2-nitrobenzoic acid (ε=13,600 cm⁻¹M⁻¹). The linker concentration was determined to be the difference between the buffer and the Y104D-MCC sample, which was approximately an average of 4.5 SMCC molecules per antibody.

The Y104D-MCC-DM1 conjugate, designated Y104D-DM1 was generated by mixing 1.7 equivalents of 10 mM DM1-SH in DMA to Y104D-MCC, diluted to 10 mg/mL Conjugate Buffer 1. DMA was added to yield a final DMA concentration of 10% v/v, and the reaction mixture was incubated overnight at room temperature (25° C.). The Drug Antibody Ratio (DAR) of Y104D-MCC-DM1 was calculated using the absorbance at 252 nm and 280 nm and an extinction coefficient of ε=1.48 mg⁻¹ cm⁻¹. The average number of DM1 molecule per antibody was 4.6.

Aggregation also was monitored by SEC-HPLC after each conjugation step. Prior to conjugation, the Y104D antibody contained about 1% high MW protein. The Y104D-MCC-DM1 conjugate typically contained about 1-15% high MW protein. The final Y104D-DM1 conjugate typically contained about 1-15% high MW protein.

b. Tumor Growth Inhibition Activity of Y104D-DM1

Male, athymic NCr-nu/nu mice were inoculated with a 0.1 mL subcutaneous (SC) injection of 3.3×10⁶ A431 epidermoid carcinoma cells (ATCC CRL1555) suspended in RPMI-1640 medium into their right flanks. When the tumors reached a size of ˜100 mm³, the animals were randomized into five study groups: (1) Group 1—vehicle (8.48 mg/mL Sodium Chloride, 1.88 mg/mL Sodium Phosphate Dibasic Heptahydrate, 0.42 mg/mL Sodium Phosphate Monobasic Monohydrate in sterile water) (n=8), (2) Group 2—unconjugated Y104D, administered at 5 mg/kg body weight twice weekly, by intraperitoneal injection, on days 0, 3, 7, and 10 (n=8), (3) Group 3—Y104D-DM1, administered at 1.0 mg/kg body weight by a single intravenous dose on day 0 (n=6), (4) Group 4—Y104D-DM1, administered at 5.0 mg/kg body weight by a single intravenous dose on day 0 (n=6), or (5) Group 5—Y104D-DM1, administered at 10.0 mg/kg body weight by a single intravenous dose on day 0 (n=6). Tumor growth was measured, as described above in Example 10, on days 0, 3, 7, 10 and 13. Tumor Growth Inhibition (TGI) was calculated as described above, based on measurements on day 13.

The results are set forth in Table 38 below. The results show there was a decreased rate of tumor growth in all animals treated with Y104D or Y104D-DM1 compared to vehicle alone. Treatment with Y104D alone, which was administered twice weekly, resulted in 26.5% TGI at day 13. The Y104D-DM1 conjugates, which were only administered one time by a single intravenous injection, resulted in a TGI at day 13 that were 32.6%, 10.0% and 20.4% for 1.0 mg/kg, 5.0 mg/kg and 10.0 mg/kg, respectively. Based on 2way-ANOVA with Dunnet post test, the observed TGI of the Y104D-DM1 treatment groups were not significantly different from one another.

TABLE 38
Y104D-DM1 Effect on Tumor Growth

| | Vehicle | | Y104D | | Y104D-DM1 | | | | | |
|---|---|---|---|---|---|---|---|---|---|---|
| Time | — | | 5.0 mg/kg | | 1.0 mg/kg | | 5.0 mg/kg | | 10.0 mg/kg | |
| (Days) | AVG | StDev | AVG | StDev | AVG | StDev | AVG | StDev | AVG | StDev |
| 0 | 136.96 | 36.05 | 132.25 | 31.24 | 144.48 | 42.24 | 135.68 | 35.68 | 135.09 | 37.01 |
| 3 | 371.98 | 121.08 | 227.91 | 77.18 | 307.83 | 113.46 | 325.16 | 53.79 | 265.30 | 84.06 |
| 7 | 702.80 | 194.15 | 469.96 | 162.93 | 479.39 | 176.36 | 542.92 | 96.45 | 460.42 | 93.48 |
| 10 | 842.46 | 252.91 | 671.37 | 249.11 | 669.94 | 234.76 | 744.05 | 236.33 | 672.79 | 117.07 |
| 13 | 1054.35 | 273.15 | 806.44 | 372.34 | 762.95 | 319.13 | 961.04 | 239.01 | 865.22 | 214.77 |

### 2. HC-Y104D Variant Anti-EGFR Antibody Conjugated to Monomethyl Auristatin E (MMAE) (Y104D-MMAE)

a. Generation of Y104D-Mc-VcPAB-MMAE (Y104D-MMAE)

Y104D antibody, generated as described in Example 2, was conjugated to MMAE via the cleavable linker maleimidocaproyl-valine-citruline-p-aminobenzyl linker (maleimidocaproyl-vcPAB-MMAE) as described in Francisco et al. Blood 102:1458-1465 (2003). Endogenous disulfides within the antibody were briefly reduced and conjugated to the maleimidocaproyl moiety to create the antibody-drug conjugate, designated Y104D-MMAE. The final conjugated product had a drug:antibody (DAR) ratio of approximately 4 as assessed by hydrophobic interaction chromatography. Typically, high molecular weight material corresponded to approximately 1-2% of the total prep. In addition, the unconjugated protein corresponded to approximately 1-2% of the intact IgG.

b. Tumor Growth Inhibition Activity of Y104D-MMAE

Mice with A431 xenograft tumors were generated as described above, except the tumors were allowed to reach a size of ˜350 mm³ before treatment. When the tumors reached a size of ˜350 mm³, the animals were randomized into four study groups: (1) Group 1—vehicle (8.48 mg/mL Sodium Chloride, 1.88 mg/mL Sodium Phosphate Dibasic Heptahydrate, 0.42 mg/mL Sodium Phosphate Monobasic Monohydrate in sterile water) (n=6), (2) Group 2—Y104D-MMAE, administered at 1.5 mg/kg body weight (n=6), (3) Group 3—Y104D-MMAE, administered at 5 mg/kg body weight (n=6), and (4) Group 4—Y104D-MMAE, administered at 15 mg/kg body weight (n=6). Due to the increased starting size of the tumors, all treatments were administered by intravenous injection, biweekly on days 0, 3, 7, and 10. Tumor growth was measured as described above on days −1, 2, 6, 10, 14 and 17.

The results are set forth in Table 39 below. Mice administered vehicle only exhibited progressive tumor growth over the course of the study. Mice receiving 1.5 mg/kg Y104D-MMAE exhibited a decreased rate in tumor growth compared to the vehicle control. At the higher doses of Y104D-MMAE, there was a substantially reduced tumor growth compared to vehicle control. For example, the tumors of mice that were administered 5 or 15 mg/kg Y104D-MMAE exhibited tumor growth reduction with regression of tumor size back to the baseline volume. Tumor growth inhibition (TGI) at Day 17, relative to vehicle, was 34.2% for 1.5 mg/kg Y104D-MMAE, 96.3% for 5 mg/kg Y104D-MMAE; and 100.9% for 15 mg/kg Y104D-MMAE. These results show that despite a similar potency of the DM1 and MMAE warheads, tumor growth inhibition mediated by Y104D-MMAE conjugates is substantially greater than for Y104D-DM1 conjugates (above).

TABLE 39
Y104D-MMAE Effect on Tumor Growth

| | Vehicle | | Y104D-MMAE | | | | | |
|---|---|---|---|---|---|---|---|---|
| Time | — | | 1.5 mg/kg | | 5 mg/kg | | 15 mg/kg | |
| (Days) | AVG | StDev | AVG | StDev | AVG | StDev | AVG | StDev |
| −1 | 364.54 | 66.04 | 356.82 | 59.04 | 358.84 | 65.57 | 357.65 | 69.35 |
| 2 | 626.74 | 147.98 | 520.37 | 165.34 | 568.76 | 143.75 | 586.35 | 145.36 |
| 6 | 977.94 | 163.14 | 704.14 | 292.50 | 543.00 | 120.95 | 484.38 | 98.17 |
| 10 | 1190.49 | 164.37 | 922.94 | 488.02 | 499.82 | 102.25 | 386.04 | 101.80 |
| 14 | 1539.80 | 269.41 | 1106.85 | 635.47 | 438.17 | 81.56 | 374.90 | 93.21 |
| 17 | 1888.41 | 471.35 | 1360.03 | 906.79 | 415.13 | 53.15 | 344.23 | 109.79 |

### 3. HC-Y104D Variant Anti-EGFR Antibody Conjugated to Monomethyl Auristatin F (MMAF) (Y104D-MMAF)

a. Generation of Y104D-Mc-MMAF (Y104D-MMAF)

Y104D antibody, generated as described in Example 2, was conjugated to MMAF, substantially as describe in part 2 above, except using MMAF conjugated to the non-cleavable linker, maleimidocaproyl (i.e., MC-MMAF). The final conjugated product had a drug:antibody ratio (DAR) of approximately 4 as assessed by hydrophobic interaction chromatography (HIC-HPLC). Typically, high molecular weight material corresponded to approximately 6.38% of the total prep. In addition, the unconjugated protein corresponded to approximately 4.58% of the intact IgG.

b. Tumor Growth Inhibition Activity of Y104D-MMAF

Mice with A431 xenograft tumors were generated as described above in part 2. When the tumors reached a size of ˜350 mm³, the animals were randomized into four study groups: (1) Group 1—vehicle (8.48 mg/mL Sodium Chloride, 1.88 mg/mL Sodium Phosphate Dibasic Heptahydrate, 0.42 mg/mL Sodium Phosphate Monobasic Monohydrate in sterile water) (n=6), (2) Group 2—Y104D-MMAF, administered at 1.5 mg/kg body weight (n=6), (3) Group 3—Y104D-MMAF, administered at 5 mg/kg body weight (n=6), and (4) Group 4—Y104D-MMAF, administered at 15 mg/kg body weight (n=6). Due to the increased starting size of the tumors, all treatments were administered by intravenous injection, biweekly on days 0, 3, 7, 11, and 16. Tumor growth was measured as described above on days −1, 2, 6, 10, 14, and 17.

The results are set forth in Table 40 below. Mice administered vehicle only exhibited progressive tumor growth over the course of the study. Mice receiving 1.5 mg/kg Y104D-MMAF exhibited a decreased rate in tumor growth compared to the vehicle control. At the higher doses of Y104D-MMAF, there was a substantially reduced tumor growth compared to vehicle control. For example, the tumors of mice that were administered 5 or 15 mg/kg Y104D-MMAF exhibited tumor growth reduction, followed by regression of tumor size back to approximately the baseline volume.

These results show that despite a reduced potency of the MMAF, compared to MMAE and DM1 warheads, tumor growth inhibition mediated by Y104D-MMAF conjugates is similar to that observed for Y104D-MMAE and substantially greater than for Y104D-DM1 conjugates (see above).

TABLE 40
Y104D-MMAF Effect on Tumor Growth

| | Vehicle | | Y104D-MMAF | | | | | |
|---|---|---|---|---|---|---|---|---|
| Time | — | | 1.5 mg/kg | | 5 mg/kg | | 15 mg/kg | |
| (Days) | AVG | StDev | AVG | StDev | AVG | StDev | AVG | StDev |
| −1 | 364.54 | 66.04 | 355.74 | 52.32 | 360.60 | 63.66 | 361.00 | 66.58 |
| 2 | 626.74 | 147.98 | 516.25 | 60.19 | 474.03 | 157.14 | 461.46 | 156.86 |
| 6 | 977.94 | 163.14 | 843.50 | 109.31 | 586.98 | 105.96 | 435.65 | 167.30 |
| 10 | 1190.49 | 164.37 | 1030.08 | 159.40 | 472.59 | 162.09 | 383.00 | 168.05 |
| 14 | 1539.80 | 269.41 | 1304.81 | 263.41 | 429.65 | 194.89 | 334.94 | 160.15 |
| 17 | 1888.41 | 471.35 | 1592.75 | 396.78 | 364.09 | 233.05 | 370.72 | 179.96 |

### Example 19

### Effect of Y104D-MMAF Antibody-Drug Conjugates (ADCs) on Tumor Growth of KRAS Mutated, EGFR+MDA MB 231 Triple Negative Breast Cancer (TNBC)Tumors In Vivo

An MDA-MB-231M human breast tumor xenograft model, which is a model of KRAS mutated, EGFR+ tumors, was used to assess tumor growth inhibition activity of Y104D-MMAF antibody drug conjugates (ADCs). Y104D-MMAF ADCs were generated as described in Example 18. The tumor growth inhibition activity of Y104D-MMAF was compared to unconjugated Cetuximab.

MDA-MB-231-luc-D2H2LN triple negative breast cancer (TNBC) cells (Caliper Life Sciences) were grown under standard conditions to a sub-confluent growth stage of 80% to 90% confluency. The cells were harvested, washed 2× with sterile HBSS, counted, and diluted to 5.0×10⁷ cells/mL with HBSS. 0.1 mL of the cell suspension (5.0×10⁶ cells) were implanted in the mammary fat pad of 4- to 6-week old, female athymic NCr-nu/nu mice. When the tumors reached a size of ˜450 mm³, the animals were randomized into three (3) study groups, as follows: HBSS (vehicle control); 30 mg/kg Cetuximab control; and 30 mg/kg huY104D-MMAF. Mice received an intravenous injection of the treatment article at days 0, 3, 7, 10, 14, and 17. Tumor growth was measured as described above on the days of treatment.

The results are set forth in Table 41. Mice administered vehicle only exhibited progressive tumor growth over the course of the study. Cetuximab treatment had no effect on tumor growth compared to vehicle-treated control animals, indicating that the antibody was not sufficient to support tumor growth inhibition in this model. In contrast, mice administered Y104D-MMAF exhibited substantially reduced tumor growth over the course of the study compared to the vehicle control, with sustained tumor volume at approximately baseline levels achieved at all tested time points.

TABLE 41
Tumor Growth Following huY104D-MMAF Treatment

| | Dose Groups | | | | | |
|---|---|---|---|---|---|---|
| | | | Cetuximab | | Y104D-MMAF | |
| Time | Vehicle | | (30 mg/kg) | | (30 mg/kg) | |
| (Days) | AVG | StDev | AVG | StDev | AVG | StDev |
| 0 | 431.64 | 113.29 | 473.33 | 82.32 | 447.56 | 113.20 |
| 3 | 620.96 | 101.27 | 655.82 | 139.47 | 522.91 | 165.07 |
| 7 | 880.90 | 191.66 | 1037.62 | 338.26 | 587.10 | 188.62 |
| 10 | 1150.54 | 158.12 | 1180.97 | 273.96 | 546.76 | 226.97 |
| 14 | 1539.59 | 210.55 | 1459.15 | 195.48 | 484.80 | 215.53 |
| 17 | 1824.12 | 317.48 | 1964.10 | 552.83 | 515.00 | 334.31 |

### Example 20

### Effect of Humanized Y104D-MMAE and Y104E-MMAE Antibody-Drug Conjugates (ADCs) on Tumor Growth of KRAS Mutated, EGFR+ Tumors In Vivo

An HT29 tumor xenograft model and MDA-MB-231M human breast tumor xenograft model, which are both models of KRAS mutated, EGFR+ tumors, were used to assess tumor growth inhibition activity of antibody drug conjugates (ADCs) of variant Y104D or Y104E anti-EGFR antibodies. Humanized Y104D and humanized Y104E antibodies were prepared as described in Example 4.3, and conjugated to MMAE as described in Example 18 to generate the ADCs designated huY104D-MMAE and huY104E-MMAE. The tumor growth inhibition activity of each ADC was compared to chimeric Y104D-MMAE conjugate generated in Example 18.

### 1. HT29 Tumor Xenograft Model

To generate HT29 tumor xenografts, male, 4- to 6-week old, athymic NCr-nu/nu mice were inoculated with a 0.1 mL subcutaneous (SC) injection of 5.0×10⁶ HT29 colorectal carcinoma cells (ATCC HTB-38) suspended in HBSS into their right flanks. When the tumors reached a size of ˜300 mm³, the animals were randomized into ten (10) study groups as follows: HBSS (vehicle control); 10 mg/kg chimeric Y104D-MMAE; or 3, 6, 10, or 30 mg/kg huY104D-MMAE; or 3, 6, 10, or 30 mg/kg huY104E-MMAE. Mice received an intravenous injection of each test article twice weekly at days 0, 4, 7, 11, 14, 18, 21, and 25. Tumor growth was measured as described above on the days of treatment. Animals were sacrificed when the tumor volume increased to or exceeded about 2000 mm³

The results are set forth in Table 42 (huY104D-MMAE) and Table 43 (huY104E-MMAE) below. For each Table of results, the Tables also set forth the tumor volume after administration of vehicle control or chimeric Y104D-MMAE at each time point. Mice administered vehicle only exhibited progressive tumor growth over the course of the study. Due to the extent of tumor growth, mice receiving vehicle control were sacrificed after day 14.

TABLE 42
Tumor Growth Following huY104D-MMAE Treatment

| | | Y104D- | | | | |
|---|---|---|---|---|---|---|
| Time | Vehicle | MMAE | huY104D-MMAE | | | |
| (Days) | — | 10 mg/kg | 3 mg/kg | 6 mg/kg | 10 mg/kg | 30 mg/kg |
| −1 | 288.6 ± 90.7 | 286.6 ± 74.2 | 299.7 ± 87.2 | 299.0 ± 97.7 | 294.8 ± 90.9 | 303.1 ± 108.9 |
| 4 | 556.1 ± 204.1 | 374.2 ± 112.0 | 560.8 ± 191.7 | 398.7 ± 108.6 | 562.6 ± 122.2 | 352.1 ± 114.1 |
| 7 | 746.3 ± 352.4 | 362.7 ± 111.7 | 736.3 ± 275.5 | 517.6 ± 207.7 | 599.0 ± 123.7 | 350.2 ± 206.8 |
| 11 | 949.5 ± 336.7 | 271.0 ± 81.1 | 914.5 ± 429.4 | 428.9 ± 202.7 | 434.6 ± 148.2 | 188.3 ± 121.6 |
| 14 | 1297.5 ± 634.5 | 173.3 ± 44.9 | 1058.9 ± 604.2 | 373.1 ± 191.0 | 361.8 ± 164.6 | 146.6 ± 135.5 |
| 18 | | 160.6 ± 48.1 | 1341.7 ± 875.1 | 358.9 ± 223.6 | 338.0 ± 204.1 | 122.4 ± 92.9 |
| 21 | | 168.2 ± 36.0 | | 407.8 ± 232.8 | 346.4 ± 195.2 | 95.2 ± 77.3 |
| 25 | | 168.6 ± 40.6 | | 449.6 ± 263.3 | 372.4 ± 193.9 | 115.3 ± 100.5 |

TABLE 43
Tumor Growth Following huY104E-MMAE Treatment

| | | Y104D- | | | | |
|---|---|---|---|---|---|---|
| Time | Vehicle | MMAE | huY104E-MMAE | | | |
| (Days) | — | 10 mg/kg | 3 mg/kg | 6 mg/kg | 10 mg/kg | 30 mg/kg |
| −1 | 288.6 ± 90.7 | 286.6 ± 74.2 | 300.9 ± 92.1 | 300.8 ± 97.6 | 307.4 ± 91.9 | 292.8 ± 86.3 |
| 4 | 556.1 ± 204.1 | 374.2 ± 112.0 | 590.2 ± 214.3 | 509.2 ± 170.7 | 552.6 ± 264.7 | 472.0 ± 192.2 |
| 7 | 746.3 ± 352.4 | 362.7 ± 111.7 | 893.8 ± 539.5 | 654.5 ± 329.7 | 583.5 ± 288.5 | 542.1 ± 232.9 |
| 11 | 949.5 ± 336.7 | 271.0 ± 81.1 | 1108.7 ± 673.3 | 721.2 ± 399.8 | 481.0 ± 307.0 | 418.0 ± 218.5 |
| 14 | 1297.5 ± 634.5 | 173.3 ± 44.9 | 1234.7 ± 768.0 | 849.1 ± 679.1 | 421.0 ± 301.1 | 324.7 ± 177.4 |
| 18 | | 160.6 ± 48.1 | | | 395.2 ± 255.3 | 286.7 ± 157.7 |
| 21 | | 168.2 ± 36.0 | | | 386.0 ± 277.0 | 249.1 ± 126.1 |
| 25 | | 168.6 ± 40.6 | | | 359.8 ± 244.9 | 209.8 ± 103.6 |

The results in Tables 42 and 43 show that administration of 10 mg/kg Y104D-MMAE to mice inhibited tumor growth compared to the vehicle control and resulted in tumor regression below the baseline tumor volume by day 11 that was sustained for the duration of the study. The results also show that mice receiving humanized forms of the ADC conjugates, huY104D-MMAE or huY104E-MMAE, also exhibited a strong anti-tumor response with tumor regression.

For example, as shown in Table 42, mice receiving huY104D-MMAE exhibited dose-dependent tumor growth inhibition, although the extent of tumor inhibition activity was slightly less than for the non-humanized chimeric Y104D-MMAE at the equivalent dose of 10 mg/kg, but was substantially the same or greater at the 30 mg/kg dose. For example, at doses of 6 or 10 mg/kg, mice treated with huY104D-MMAE exhibited reduced tumor growth compared to mice treated with vehicle control, which resulted in tumor regression to approximately baseline tumor volume. At the higher dose of 30 mg/kg, the presence of huY104D-MMAE inhibited tumor growth and resulted in tumor regression to less than baseline levels, resulting in tumor volumes, at day 25, that were less than half the starting (baseline) tumor volume.

Similar to the results with the huY104D-MMAE, the results in Table 43 show that mice receiving huY104E-MMAE also exhibited dose-dependent tumor inhibition. In this experiment, the extent of anti-tumor response of huY104E-MMAE also was slightly less than for the non-humanized chimeric Y104D-MMAE at all doses tested. For example, mice receiving 10 or 30 mg/kg exhibited reduced tumor growth compared to the vehicle-treated animals, which resulted in tumor regression to about baseline.

### 2. MDA MB 231 Triple Negative Breast Cancer (TNBC)

A similar study to that described above also was performed in a breast cancer xenograft model generated using the MDA MB 231M xenograft model described in Example 19 above. When the tumors reached a size of ˜400 mm³, the animals were randomized into ten (10) study groups, each containing 6 mice, as follows: HBSS (vehicle control); 10 mg/kg chimeric Y104D-MMAE control; 3, 6, 10, or 30 mg/kg huY104D-MMAE; or 3, 6, 10, or 30 mg/kg huY104E-MMAE. Mice received an intravenous injection of the treatment article at days 0, 3, 6, 9, 13, 16, 20, and 23. Tumor growth was measured as described above on the days of treatment.

The results are set forth in Table 44 (huY104D-MMAE) and Table 45 (huY104E-MMAE). For each Table of results, the Tables also set forth the tumor volume after administration of vehicle control or chimeric Y104D-MMAE at each time point. Mice administered vehicle only exhibited progressive tumor growth over the course of the study. Due to the extent of tumor growth, mice receiving vehicle control were sacrificed after day 13.

TABLE 44
Breast Tumor Growth Following huY104D-MMAE Treatment

| | | Y104D- | | | | |
|---|---|---|---|---|---|---|
| Time | Vehicle | MMAE | huY104D-MMAE | | | |
| (Days) | — | 10 mg/kg | 3 mg/kg | 6 mg/kg | 10 mg/kg | 30 mg/kg |
| −1 | 397.8 ± 35.9 | 400.7 ± 28.3 | 404.0 ± 31.5 | 403.6 ± 33.4 | 398.3 ± 39.9 | 396.1 ± 42.9 |
| 3 | 565.3 ± 121.0 | 513.9 ± 118.1 | 675.3 ± 159.1 | 614.9 ± 153.7 | 535.4 ± 99.5 | 465.2 ± 127.5 |
| 6 | 783.0 ± 148.7 | 478.4 ± 112.3 | 668.6 ± 137.1 | 602.6 ± 130.8 | 480.6 ± 114.7 | 418.6 ± 95.4 |
| 9 | 1026.9 ± 208.8 | 354.3 ± 84.2 | 725.2 ± 135.2 | 543.5 ± 185.6 | 308.7 ± 52.8 | 255.5 ± 79.6 |
| 13 | 1398.3 ± 442.9 | 223.5 ± 91.7 | 901.4 ± 146.4 | 561.3 ± 221.1 | 122.4 ± 43.5 | 154.2 ± 26.6 |
| 16 | | 202.3 ± 145.3 | 1301.3 ± 456.5 | 617.1 ± 246.8 | 77.3 ± 69.1 | 90.7 ± 56.7 |
| 20 | | 262.7 ± 252.8 | | 811.1 ± 349.8 | 67.3 ± 69.5 | 47.6 ± 44.2 |
| 23 | | 332.9 ± 329.7 | | 958.5 ± 378.5 | 57.5 ± 38.2 | 28.8 ± 22.9 |

TABLE 45
Breast Tumor Growth Following huY104E-MMAE Treatment

| | | Y104D- | | | | |
|---|---|---|---|---|---|---|
| Time | Vehicle | MMAE | huY104E-MMAE | | | |
| (Days) | — | 10 mg/kg | 3 mg/kg | 6 mg/kg | 10 mg/kg | 30 mg/kg |
| −1 | 397.8 ± 35.9 | 400.7 ± 28.3 | 398.4 ± 39.7 | 398.4 ± 37.8 | 403.6 ± 36.2 | 401.0 ± 30.4 |
| 3 | 565.3 ± 121.0 | 513.9 ± 118.1 | 488.7 ± 77.6 | 523.7 ± 150.6 | 516.5 ± 81.6 | 458.2 ± 66.6 |
| 6 | 783.0 ± 148.7 | 478.4 ± 112.3 | 532.6 ± 95.7 | 525.9 ± 73.3 | 461.1 ± 89.9 | 496.5 ± 94.8 |
| 9 | 1026.9 ± 208.8 | 354.3 ± 84.2 | 543.8 ± 129.6 | 571.2 ± 187.6 | 323.5 ± 39.8 | 343.8 ± 57.8 |
| 13 | 1398.3 ± 442.9 | 223.5 ± 91.7 | 771.0 ± 322.7 | 581.2 ± 249.6 | 232.8 ± 133.6 | 172.5 ± 73.7 |
| 16 | | 202.3 ± 145.3 | 871.5 ± 462.2 | 664.8 ± 480.3 | 193.2 ± 176.7 | 116.1 ± 62.0 |
| 20 | | 262.7 ± 252.8 | 1167.1 ± 446.5 | 865.0 ± 577.1 | 173.5 ± 189.2 | 84.3 ± 53.2 |
| 23 | | 332.9 ± 329.7 | 1416.7 ± 465.4 | 1134.6 ± 741.1 | 201.7 ± 233.3 | 50.5 ± 34.2 |

The results, as shown in Tables 44 and 45, demonstrate that administration of 10 mg/kg Y104D-MMAE to mice inhibited tumor growth compared to the vehicle control and resulted in tumor regression to below the baseline tumor volume by day 9 that was sustained for the duration of the study.

Similar to the results above in the HT29 xenograft tumor model, the results also show that mice receiving humanized forms of the ADC conjugates, huY104D-MMAE or huY104E-MMAE, also exhibited a strong anti-tumor response with tumor regression. At the lower doses of each of huY104D-MMAE and huY104E-MMAE tested of 3 mg/kg and 6 mg/kg, a reduction in tumor growth compared to vehicle control was observed, but no tumor regression developed. In contrast, like chimeric Y104D-MMAE, both huY104D-MMAE and huY104E-MMAE resulted in tumor regression upon administration of tested doses of 10 mg/kg and 30 mg/kg.

For example, as shown in Table 44, mice treated with 10 or 30 mg/kg huY104D-MMAE exhibited reduced tumor growth compared to the control and resulted in continued tumor regression beginning at day 6 of the study. At completion of the study, the remaining tumor volume in mice treated with huY104D-MMAE was only about 14% (10 mg/kg) or 7% (30 mg/kg) of the initial tumor volume (see Table 42). Similar results were observed in mice administered 10 mg/kg and 30 mg/kg doses of huY104E-MMAE as shown in Table 45. At completion of the study, the remaining tumor volume in mice treated with huY104E-MMAE was about 50% (10 mg/kg) or 13% (30 mg/kg) of the initial tumor volume (Table 45).

### 3. Conclusion

These results confirm that the Y104D-MMAE conjugate, and the humanized forms huY104D-MMAE and huY104E-MMAE, exhibit a strong anti-tumor response in KRAS mutated, EGFR+ tumor model. The anti-tumor response of each of the tested antibodies achieves tumor growth regression.

### Example 21

### Assessment of pH-Dependent Growth of Tumor Cells Versus Keratinocytes in the Presence of Cetuximab-MMAE or Y104D-MMAE Antibody-Drug Conjugates (ADCs)

The pH-dependent effect of anti-EGFR ADC conjugates on growth of EGFR-expressing cells was assessed. Specifically, cell growth of A431 tumor cells at pH 6.8 or cell growth of keratinocyte cells at pH 7.4, each in the presence of Cetuximab-MMAE or Y104D-MMAE, was assessed and compared. Y104D-MMAE was generated as described in Example 18. Cetuximab-MMAE was generated using similar procedures.

### 1. Method

The day before treatment, A431 cells (ATCC CRL 1555) or a human neonatal keratinocyte cell line (Invitrogen C-001-5C) were plated in wells of a 96 well plate (BD Falcon #35-3072) at about 2,000 cells/well in 100 μL complete media that had been adjusted to appropriate pH. Keratinocytes were tested in EpiLife supplemented with EpiLife Defined Growth Supplement (Life Technologies) at pH 7.4 and A431 cells were tested in RPMI 1640+10% FBS buffered with MES (pH6.8). The pH of MES-buffered RPMI 1640+10% FBS was initially set at pH 7.4 and media reached pH 6.8 after 2 hours in the CO₂ incubator. Hence, cell growth of A431 cells was assessed at pH 6.8 and cell growth of keratinocyte cells was assessed at pH 7.4. The cells were untreated or treated with 1:3 serial dilutions of each of the ADCs that were added to achieve concentrations between 0.9 μg/mL and 0.00001 μg/mL for A431 cells. Each of the ADCs were tested at concentrations between 90 μg/mL and 0.001 μg/mL in Keratinocytes. Each condition was assayed in 3 replicate wells (i.e., n=3 per condition). Cells were incubated for 5 days at 37° C. in a humidified atmosphere of 5% CO₂ incubator. Cell growth was measured by CellTiter-Glo® Luminescent Cell Viability Assay (Promega Cat# G-7571) according to the manufacturer's instructions. The percent cell growth inhibition (CGI) was determined as the percentage of the decrease in surviving cells relative to untreated cells. The IC50 value was defined as the drug concentration needed to inhibit 50% of the cell growth compared to growth of the untreated control cells.

### 2. Results

The results showed that Cetuximab-MMAE and Y104D-MMAE exhibited inhibition of cell growth of A431 cells, which was virtually identical between the tested agents. When plotted with the concentration on the x-axis (LOG (mg/mL) and % cell growth inhibition on the y-axis, treatment with Cetuximab-MMAE and Y104D-MMAE exhibited overlapping, sigmoidal, concentration-dependent cell growth inhibition. Approximately 50% CGI was observed for both ADCs at approximately 0.001 μg/mL, and up to approximately 80% CGI was observed for both ADCs at the higher concentrations tested.

In contrast, Y104D-MMAE exhibited less keratinocyte growth inhibition compared to Cetuximab-MMAE. When plotted with the concentration on the x-axis (LOG (μg/mL) and % cell growth inhibition on the y-axis, treatment with Cetuximab-MMAE and Y104D-MMAE exhibited sigmoidal, concentration dependent cell growth inhibition, but the curve for the Y104D-MMAE treatment was shifted to indicate significantly reduced growth inhibition. For example, Cetuximab-MMAE exhibited approximately 50% CGI at a concentration of approximately 0.1 μg/mL, whereas to achieve 50% CGI, 1 μg/mL Y104D-MMAE was required. At the maximum doses, Cetuximab achieved about 80% CGI and Y104D-MMAE exhibited a CGI of about 70%.

Therefore, these results confirm that the MMAE ADC conjugate of Y104D anti-EGFR retains the pH-dependent activity of Y104D anti-EGFR, such that the Y104D-MMAE exhibits less cell growth inhibition activity of skin keratinocytes than the A431 tumor cells.

### Example 22

### Assessment of pH-Dependent Growth of Tumor Cells Versus Keratinocytes in the Presence of Chimeric Y104D-MMAE or Humanized Y104 Variant Antibody-Drug Conjugates (ADCs)

To confirm the activity of ADC conjugates of humanized anti-EGFR variants huY104D-MMAE and huY104E-MMAE, the pH-dependent effect of each on growth of EGFR-expressing cells was assessed and compared to chimeric Y104D-MMAE using procedures substantially the same as described in Example 20. Specifically, cell growth of HT29 tumor cells (ATCC HTB-38) at pH 6.8, MDA-MB-231 tumor cells (ATCC HTB-26) at pH 6.8 or keratinocyte cells at pH 7.4 was assessed, each in the presence of Y104D-MMAE, huY104D-MMAE or huY104E-MMAE. Each of the tested conjugates was generated using procedures as described in Example 18. In addition, for studies assessing cell growth of keratinocytes, the non Adcetris (brentuximab vedotin, an non-target antibody conjugated MMAE; obtained from a Commercial Pharmacy) also was tested for cell growth inhibition as a negative control.

1. Method

The cell growth assays were performed using substantially the same procedures described in Example 21, except the ADCs were serially diluted 1:3 to achieve concentrations between 200 and 0.03 nM. HT29 cells and MDA-MB-231 cells were cultured and assayed in the presence of MES-buffered RPMI+10% FBS (pH 6.8), while keratinocytes were cultured and assayed in Epilife (pH 7.4)

2. Results

Treatment with each of chimeric Y104D-MMAE, huY104D-MMAE or huY104E-MMAE resulted in similar levels of dose-dependent cell growth inhibition of both HT29 and MDA-MB-231 cells. For HT29 cells, the ADC treatment resulted in about 50% cell growth inhibition at a concentration of approximately 22 nM and about 80% cell growth inhibition at the maximum concentration tested. For MDA-MB-231 cells, the ADC treatment resulted in up to about 35-40% cell growth inhibition at the maximum dose tested.

Chimeric Y104D-MMAE exhibited cell growth inhibition of keratinocytes, with 50% growth inhibition achieved with 3.7 nM chimeric Y104D-MMAE. In contrast, each of the humanized variant ADCs, huY104D-MMAE and huY104E-MMAE, exhibited reduced keratinocyte growth inhibition compared to chimeric Y104D-MMAE. For example, the IC50 for cell growth inhibition of huY104D-MMAE was about 17 nM and the IC50 for cell growth inhibition of huY104E-MMAE was about 5.4 nM. In comparison, the IC50 for cell growth inhibition of the non-target antibody control, Adcetris was 50 nM.

Therefore, these results show that ADC conjugates of the humanized forms of the Y104D- and Y104E-anti-EGFR variants exhibit greater pH-dependent activity than the chimeric Y104D-MMAE conjugate. For example, while each are as effective as the chimeric Y104D-MMAE for inhibiting tumor cell growth at pH 6.8, each exhibit reduced growth inhibition of non-tumor keratinocytes at pH 7.4 compared to the chimeric Y104D-MMAE.

### Example 23

### Stability of Y104E Antibody Drug Conjugate (ADC) Formulations

HuY104E-MMAE ADCs, generated as described in Example 20, were formulated in compositions with different stabilizing agents as set forth in Table 46. Each antibody formulation, at a protein concentration of 3.75 mg/ml, was placed into 1.5 mL Eppendorf tube and subjected to 2 rounds of freeze-thaw cycles, where the tubes were frozen in dry ice/ethanol for 30 min. and then allowed to thaw at room temperature in a rack for 10 min. The samples were analyzed for protein content by measuring absorbance at 280 nm, turbidity (aggregation) by measuring absorbance at 340 nm, and opalescence (aggregation) by measuring transmittance (T %) at 580 nm, following formulation and after each freeze-thaw cycle, to access formulation stability.

The results, presented as a percent increase after the respective thaw cycle compared to the initial value, are provided in Table 46 below. The results indicate that all tested stabilizing agents result in more stable formulations than PBS alone. In particular, the results indicate PBS formulations that include 0.02% Tween 80 or 280 mM trehalose stabilizing agents are more stable than formulations with the other stabilizing agents.

TABLE 46
Y104E ADC Formulations

| | OD | | | | | |
|---|---|---|---|---|---|---|
| | 280 nm: protein | | 340 nm: turbidity | | 580 nm: Opalescence | |
| | freezing/thawing | | | | | |
| | Thaw 1 | Thaw 2 | Thaw 1 | Thaw 2 | Thaw 1 | Thaw 2 |
| | (% increase) | (% increase) | (% increase) | (% increase) | (% increase) | (% increase) |
| PBS | 1 | 2.1 | 131.3 | 462.5 | 550 | 1750 |
| PBS/3.6% glycerol | 0.7 | 0.3 | 42.9 | 42.9 | 300 | 500 |
| PBS/280 mM | 0 | 0.3 | 0 | 52.9 | −33 | 133 |
| trehalose | | | | | | |
| PBS/280 mM sucrose | 0.3 | 0.3 | 5.6 | 27.8 | 50 | 100 |
| PBS/280 mM sorbitol | 0.3 | 0.3 | 76.5 | 82.4 | 250 | 300 |
| PBS/0.02% Tween 80 | −0.7 | 0 | −33.3 | −5.6 | −50 | −50 |

Since modifications will be apparent to those of skill in the art, it is intended that this invention be limited only by the scope of the appended claims.

1. A modified anti-EGFR antibody or antigen-binding fragment thereof, comprising an amino acid replacement(s) in a variable heavy chain (VH) of an unmodified anti-EGFR antibody or antigen-binding fragment thereof corresponding to replacement with glutamic acid (E) at a position corresponding to position 104 with reference to amino acid positions set forth in SEQ ID NO: 2 or 7, wherein:
amino acid positions are identified by alignment of the variable heavy chain of the antibody with the variable heavy chain set forth in SEQ ID NO: 2 or 7;
the unmodified anti-EGFR antibody or antigen-binding fragment thereof is selected from among:
i) cetuximab or an antigen-binding fragment thereof comprising a variable heavy chain set forth in SEQ ID NO: 2 or 7 and a variable light chain set forth in SEQ ID NOS: 4, 9 or 11;
ii) an antibody or antigen-binding fragment thereof that exhibits at least 85% sequence identity to the antibody or antigen-binding fragment thereof of i); and
iii) a humanized form of i) or ii); and
the modified anti-EGFR antibody specifically binds epidermal growth factor receptor (EGFR) or a soluble fragment thereof.

2. The modified anti-EGFR antibody, or antigen-binding fragment thereof, of claim 1, comprising:
a) a variable heavy (VH) chain comprising the sequence of amino acids set forth in SEQ ID NO: 74 or 75, or a sequence of amino acids that exhibits at least 85%, 90% or 95% sequence identity to SEQ ID NO: 74 or 75; and
b) a variable light (VL) chain comprising the sequence of amino acids set forth in SEQ ID NO:4, 9 or 11, or a sequence of amino acids that exhibits at least 85%, 90% or 95% sequence identity to SEQ ID NO:4, 9 or 11.

3. The modified anti-EGFR antibody, or antigen-binding fragment thereof, of claim 1, wherein the modified antibody or antigen-binding fragment thereof is humanized and comprises the amino acid replacement with glutamic acid (E) at a position corresponding to position 104.

4. The anti-EGFR antibody, or antigen-binding fragment thereof, of claim 3, comprising:
a) a variable heavy chain comprising the sequence of amino acids set forth in SEQ ID NO: 61 or 63 or a sequence of amino acids that exhibits at least 85%, 90% or 95% sequence identity to SEQ ID NO: 61 or 63; and
b) a variable light chain comprising the sequence of amino acids set forth in SEQ ID NO: 183, 184 or 186 or a sequence of amino acids that exhibits at least 85%, 90% or 95% sequence identity to SEQ ID NO: 183, 184 or 186.

5. The modified anti-EGFR antibody, or antigen-binding fragment thereof, of claim 2, that comprises up to 1, 2, 3, 4, 5, 6, 7, 8, 9 or 10 amino acid replacements in the variable heavy chain set forth in SEQ ID NO: 74 or 75 and/or in the variable light chain set forth in SEQ ID NO: 4, 9 or 11, other than the replacement with E at a position corresponding to position 104 in the heavy chain.

6. The modified anti-EGFR antibody, or antigen-binding fragment thereof, of claim 4, that comprises up to 1, 2, 3, 4, 5, 6, 7, 8, 9 or 10 amino acid replacements in the variable heavy chain set forth in SEQ ID NO: 61 or 63 and/or in the variable light chain set forth in SEQ ID NO: 183, 184 or 186, other than the replacement with E at a position corresponding to position 104 in the heavy chain.

7. The modified anti-EGFR antibody, or antigen-binding fragment thereof, of claim 1, comprising one or more amino acid replacement(s) selected from among:
amino acid replacement(s) in a variable heavy chain of the unmodified antibody corresponding to amino acid replacement(s) selected from among V24I, V24L, V24E, S25C, S25G, S25I, S25M, S25V, S25Q, S25T, S25L, S25H, S25R, S25A, S25D, F27R, S28C, L29H, T30F, N31H, N31I, N31T, N31V, Y32T, V50L, S53G, G54D, G54S, G54R, G54C, G54P, D58M, Y59E, F63R, F63C, F63G, F63M, F63V, F63P, F63S, T64N, T64V, L67G, S68F, S68Q, D72K, D72L, D72P, D72M, D72W, N73Q, S74H, S74R, S74D, S74G, S74Y, K75H, K75G, K75W, K75P, S76I, S76V, Q77R, Q77E, R97H, T100I, T100P, Y101W, Y105V, A107N, Q111I, Q111P and Q111V with reference to SEQ ID NO:2 or 7, wherein corresponding amino acid positions are identified by alignment of the variable heavy chain of the antibody with the variable heavy chain set forth in SEQ ID NO: 2 or 7; and
amino acid replacement(s) in a variable light chain of the unmodified antibody corresponding to amino acid replacement(s) selected from among L4C, L4F, L4V, T5P, R24G, I29S, S56H and N91V with reference to SEQ ID NO:4, wherein corresponding amino acid positions are identified by alignment of the variable light chain of the antibody with the variable light chain set forth in SEQ ID NO: 4.

8. The modified anti-EGFR antibody, or antigen-binding fragment thereof, of claim 7, wherein the amino acid replacements are selected from among HC-Y104E/HC-Q111P; HC-S25C/HC-Y104E; HC-Y104E/HC-Q111P/LC-I29S; HC-S53G/HC-Y104E; HC-S53G/HC-Y104E/HC-Q111P; HC-S25V/HC-Y104E; HC-S25V/HC-Y104E/HC-Q111P; HC-S25V/HC-S53G/HC-Y104E; HC-S25V/HC-S53G/HC-Y104E/HC-Q111P; HC-T30F/HC-Y104E; HC-T30F/HC-Y104E/HC-Q111P; HC-T30F/HC-S53G/HC-Y104E; HC-T30F/HC-S53G/HC-Y104E/HC-Q111P; HC-D72L/HC-Y104E; HC-D72L/HC-Y104E/HC-Q111P; HC-S53G/HC-D72L/HC-Y104E; HC-S53G/HC-D72L/HC-Y104E/HC-Q111P; HC-Y104E/LC-I29S and HC-Y104E/HC-Q111P/LC-I29S.

9. The modified anti-EGFR antibody, or antigen-binding fragment thereof, of claim 7, comprising:
a) a variable heavy (VH) chain comprising the sequence of amino acids set forth in SEQ ID NOS: 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, or 123, or a sequence of amino acids that exhibits at least 85%, 90%, or 95% sequence identity to any of SEQ ID NOS: 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, or 123; and
b) a variable light (VL) chain comprising the sequence of amino acids set forth in SEQ ID NO:4, 9 or 11, or a sequence of amino acids that exhibits at least 85%, 90%, or 95% sequence identity to SEQ ID NO:4, 9 or 11.

10. The modified anti-EGFR antibody, or antigen-binding fragment thereof, of claim 7, comprising:
a) a variable heavy (VH) chain comprising the sequence of amino acids set forth in SEQ ID NO: 74, 75, 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, or 123, or a sequence of amino acids that exhibits at least 85%, 90% or 95% sequence identity to any of SEQ ID NOS: 74, 75, 77, 78, 80, 81, 83, 84, 86, 87, 89, 90, 92, 93, 95, 96, 98, 99, 101, 102, 104, 105, 107, 108, 110, 111, 113, 114, 116, 117, 119, 120, 122, or 123; and
b) a variable light (VL) chain comprising the sequence of amino acids set forth in SEQ ID NO: 125, 126 or 127, or a sequence of amino acids that exhibits at least 85%, 90% or 95% sequence identity to any of SEQ ID NOS: 125, 126 or 127.

11. The modified anti-EGFR antibody, or antigen-binding fragment thereof, of claim 3, comprising one or more amino acid replacement(s) selected from among:
amino acid replacement(s) in a variable heavy chain of the unmodified antibody corresponding to amino acid replacement(s) selected from among V24I, V24L, V24E, S25C, S25G, S25I, S25M, S25V, S25Q, S25T, S25L, S25H, S25R, S25A, S25D, F27R, S28C, L29H, T30F, N31H, N31I, N31T, N31V, Y32T, V50L, S53G, G54D, G54S, G54R, G54C, G54P, D58M, Y59E, F63R, F63C, F63G, F63M, F63V, F63P, F63S, T64N, T64V, L67G, S68F, S68Q, D72K, D72L, D72P, D72M, D72W, N73Q, S74H, S74R, S74D, S74G, S74Y, K75H, K75G, K75W, K75P, S76I, S76V, Q77R, Q77E, R97H, T100I, T100P, Y101W, Y105V, A107N, Q111I, Q111P and Q111V with reference to SEQ ID NO:2 or 7, wherein corresponding amino acid positions are identified by alignment of the variable heavy chain of the antibody with the variable heavy chain set forth in SEQ ID NO: 2 or 7; and
amino acid replacement(s) in a variable light chain of the unmodified antibody corresponding to amino acid replacement(s) selected from among L4C, L4F, L4V, T5P, R24G, I29S, S56H and N91V with reference to SEQ ID NO:4, wherein corresponding amino acid positions are identified by alignment of the variable light chain of the antibody with the variable light chain set forth in SEQ ID NO: 4.

12. The modified anti-EGFR antibody, or antigen-binding fragment thereof, of claim 11, wherein the amino acid replacements are selected from among HC-Y104E/HC-Q111P; HC-S25C/HC-Y104E; HC-Y104E/HC-Q111P/LC-I29S; HC-S53G/HC-Y104E; HC-S53G/HC-Y104E/HC-Q111P; HC-S25V/HC-Y104E; HC-S25V/HC-Y104E/HC-Q111P; HC-S25V/HC-S53G/HC-Y104E; HC-S25V/HC-S53G/HC-Y104E/HC-Q111P; HC-T30F/HC-Y104E; HC-T30F/HC-Y104E/HC-Q111P; HC-T30F/HC-S53G/HC-Y104E; HC-T30F/HC-S53G/HC-Y104E/HC-Q111P; HC-D72L/HC-Y104E; HC-D72L/HC-Y104E/HC-Q111P; HC-S53G/HC-D72L/HC-Y104E; HC-S53G/HC-D72L/HC-Y104E/HC-Q111P; HC-Y104E/LC-I29S and HC-Y104E/HC-Q111P/LC-I29S.

13. The anti-EGFR antibody, or antigen-binding fragment thereof, of claim 11, comprising a sequence of amino acids selected from among:
a) the variable heavy chain set forth in SEQ ID NO: 131 or 133 or a sequence of amino acids that exhibits at least 85% sequence identity to SEQ ID NO: 131 or 133, and the variable light chain set forth in SEQ ID NO: 155, 156 or 158 or a sequence of amino acids that exhibits at least 85% sequence identity to SEQ ID NO: 155, 156 or 158;
b) the variable heavy chain set forth in SEQ ID NO: 131 or 133 or a sequence of amino acids that exhibits at least 85% sequence identity to SEQ ID NO: 131 or 133, and the variable light chain set forth in SEQ ID NO: 162, 163 or 165 or a sequence of amino acids that exhibits at least 85% sequence identity to SEQ ID NO: 162, 163 or 165;
c) the variable heavy chain set forth in SEQ ID NO: 137 or 139 or a sequence of amino acids that exhibits at least 85% sequence identity to SEQ ID NO: 137 or 139, and the variable light chain set forth in SEQ ID NO: 155, 156 or 158 or a sequence of amino acids that exhibits at least 85% sequence identity to SEQ ID NO: 155, 156 or 158;
d) the variable heavy chain set forth in SEQ ID NO: 131 or 133 or a sequence of amino acids that exhibits at least 85% sequence identity to SEQ ID NO: 131 or 133, and the variable light chain set forth in SEQ ID NO: 169, 170 or 172 or a sequence of amino acids that exhibits at least 85% sequence identity to SEQ ID NO: 169, 170 or 172;
e) the variable heavy chain set forth in SEQ ID NO: 131 or 133 or a sequence of amino acids that exhibits at least 85% sequence identity to SEQ ID NO: 131 or 133, and the variable light chain set forth in SEQ ID NO: 176, 177 or 179 or a sequence of amino acids that exhibits at least 85% sequence identity to SEQ ID NO: 176, 177 or 179;
f) the variable heavy chain set forth in SEQ ID NO: 131 or 133 or a sequence of amino acids that exhibits at least 85% sequence identity to SEQ ID NO: 131 or 133 and the variable light chain set forth in SEQ ID NO: 183, 184 or 186 or a sequence of amino acids that exhibits at least 85% sequence identity to SEQ ID NO: 183, 184 or 186;
g) the variable heavy chain set forth in SEQ ID NO: 137 or 139 or a sequence of amino acids that exhibits at least 85% sequence identity to SEQ ID NO: 137 or 139, and the variable light chain set forth in SEQ ID NO: 183, 184 or 186 or a sequence of amino acids that exhibits at least 85% sequence identity to SEQ ID NO: 183, 184 or 186;
h) the variable heavy chain set forth in SEQ ID NO: 131 or 133 or a sequence of amino acids that exhibits at least 85% sequence identity to SEQ ID NO: 131 or 133, and the variable light chain set forth in SEQ ID NO: 190, 191 or 193 or a sequence of amino acids that exhibits at least 85% sequence identity to SEQ ID NO: 190, 191 or 193;
i) the variable heavy chain set forth in SEQ ID NO: 143 or 145 or a sequence of amino acids that exhibits at least 85% sequence identity to SEQ ID NO: 143 or 145, and the variable light chain set forth in SEQ ID NO: 183, 184 or 186 or a sequence of amino acids that exhibits at least 85% sequence identity to SEQ ID NO: 183, 184 or 186;
j) the variable heavy chain set forth in SEQ ID NO: 149 or 151 or a sequence of amino acids that exhibits at least 85% sequence identity to SEQ ID NO: 149 or 151, and the variable light chain set forth in SEQ ID NO: 197, 198 or 200 or a sequence of amino acids that exhibits at least 85% sequence identity to SEQ ID NO: 197, 198 or 200;
k) the variable heavy chain set forth in SEQ ID NO: 143 or 145 or a sequence of amino acids that exhibits at least 85% sequence identity to SEQ ID NO: 143 or 145, and the variable light chain set forth in SEQ ID NO: 197, 198 or 200 or a sequence of amino acids that exhibits at least 85% sequence identity to SEQ ID NO: 197, 198 or 200;
l) the variable heavy chain set forth in SEQ ID NO: 149 or 151 or a sequence of amino acids that exhibits at least 85% sequence identity to SEQ ID NO: 149 or 151, and the variable light chain set forth in SEQ ID NO: 204, 205 or 207 or a sequence of amino acids that exhibits at least 85% sequence identity to SEQ ID NO: 204, 205 or 207;
m) the variable heavy chain set forth in SEQ ID NO: 143 or 145 or a sequence of amino acids that exhibits at least 85% sequence identity to SEQ ID NO: 143 or 145, and the variable light chain set forth in SEQ ID NO: 204, 205 or 207 or a sequence of amino acids that exhibits at least 85% sequence identity to SEQ ID NO: 204, 205 or 207;
n) the variable heavy chain set forth in SEQ ID NO: 211 or 213 or a sequence of amino acids that exhibits at least 85% sequence identity to SEQ ID NO: 211 or 213, and the variable light chain set forth in SEQ ID NO: 253, 254 or 256 or a sequence of amino acids that exhibits at least 85% sequence identity to SEQ ID NO: 253, 254 or 256;
o) the variable heavy chain set forth in SEQ ID NO: 217 or 219 or a sequence of amino acids that exhibits at least 85% sequence identity to SEQ ID NO: 217 or 219, and the variable light chain set forth in SEQ ID NO: 253, 254 or 256 or a sequence of amino acids that exhibits at least 85% sequence identity to SEQ ID NO: 253, 254 or 256;
p) the variable heavy chain set forth in SEQ ID NO: 223 or 225 or a sequence of amino acids that exhibits at least 85% sequence identity to SEQ ID NO: 223 or 225, and the variable light chain set forth in SEQ ID NO: 260, 261 or 263 or a sequence of amino acids that exhibits at least 85% sequence identity to SEQ ID NO: 260, 261 or 263;
q) the variable heavy chain set forth in SEQ ID NO: 229 or 231 or a sequence of amino acids that exhibits at least 85% sequence identity to SEQ ID NO: 229 or 231, and the variable light chain set forth in SEQ ID NO: 260, 261 or 263 or a sequence of amino acids that exhibits at least 85% sequence identity to SEQ ID NO: 260, 261 or 263;
r) the variable heavy chain set forth in SEQ ID NO: 235 or 237 or a sequence of amino acids that exhibits at least 85% sequence identity to SEQ ID NO: 235 or 237, and the variable light chain set forth in SEQ ID NO: 267, 268 or 270 or a sequence of amino acids that exhibits at least 85% sequence identity to SEQ ID NO: 267, 268 or 270;
s) the variable heavy chain set forth in SEQ ID NO: 241 or 243 or a sequence of amino acids that exhibits at least 85% sequence identity to SEQ ID NO: 241 or 243, and the variable light chain set forth in SEQ ID NO: 274, 275 or 277 or a sequence of amino acids that exhibits at least 85% sequence identity to SEQ ID NO: 274, 275 or 277;
t) the variable heavy chain set forth in SEQ ID NO: 223 or 225 or a sequence of amino acids that exhibits at least 85% sequence identity to SEQ ID NO: 223 or 225, and the variable light chain set forth in SEQ ID NO: 274, 275 or 277 or a sequence of amino acids that exhibits at least 85% sequence identity to SEQ ID NO: 274, 275 or 277;
u) the variable heavy chain set forth in SEQ ID NO: 229 or 231 or a sequence of amino acids that exhibits at least 85% sequence identity to SEQ ID NO: 229 or 231, and the variable light chain set forth in SEQ ID NO: 274, 275 or 277 or a sequence of amino acids that exhibits at least 85% sequence identity to SEQ ID NO: 274, 275 or 277;
v) the variable heavy chain set forth in SEQ ID NO: 235 or 237 or a sequence of amino acids that exhibits at least 85% sequence identity to SEQ ID NO: 235 or 237, and the variable light chain set forth in SEQ ID NO: 281, 282 or 284 or a sequence of amino acids that exhibits at least 85% sequence identity to SEQ ID NO: 281, 282 or 284;
w) the variable heavy chain set forth in SEQ ID NO: 247 or 249 or a sequence of amino acids that exhibits at least 85% sequence identity to SEQ ID NO: 247 or 249, and the variable light chain set forth in SEQ ID NO: 281, 282 or 284 or a sequence of amino acids that exhibits at least 85% sequence identity to SEQ ID NO: 281, 282 or 284;
x) the variable heavy chain set forth in SEQ ID NO: 223 or 225 or a sequence of amino acids that exhibits at least 85% sequence identity to SEQ ID NO: 223 or 225, and the variable light chain set forth in SEQ ID NO: 281, 282 or 284 or a sequence of amino acids that exhibits at least 85% sequence identity to SEQ ID NO: 281, 282 or 284;
y) the variable heavy chain set forth in SEQ ID NO: 229 or 231 or a sequence of amino acids that exhibits at least 85% sequence identity to SEQ ID NO: 229 or 231, and the variable light chain set forth in SEQ ID NO: 281, 282 or 284 or a sequence of amino acids that exhibits at least 85% sequence identity to SEQ ID NO: 281, 282 or 284;
z) the variable heavy chain set forth in SEQ ID NO: 235 or 237 or a sequence of amino acids that exhibits at least 85% sequence identity to SEQ ID NO: 235 or 237, and the variable light chain set forth in SEQ ID NO: 288, 289 or 291 or a sequence of amino acids that exhibits at least 85% sequence identity to SEQ ID NO: 288, 289 or 291;
aa) the variable heavy chain set forth in SEQ ID NO: 247 or 249 or a sequence of amino acids that exhibits at least 85% sequence identity to SEQ ID NO: 247 or 249, and the variable light chain set forth in SEQ ID NO: 288, 289 or 291 or a sequence of amino acids that exhibits at least 85% sequence identity to SEQ ID NO: 288, 289 or 291;
bb) the variable heavy chain set forth in SEQ ID NO: 223 or 225 or a sequence of amino acids that exhibits at least 85% sequence identity to SEQ ID NO: 223 or 225, and the variable light chain set forth in SEQ ID NO: 288, 289 or 291 or a sequence of amino acids that exhibits at least 85% sequence identity to SEQ ID NO: 288, 289 or 291;
cc) the variable heavy chain set forth in SEQ ID NO: 229 or 231 or a sequence of amino acids that exhibits at least 85% sequence identity to SEQ ID NO: 229 or 231, and the variable light chain set forth in SEQ ID NO: 288, 289 or 291 or a sequence of amino acids that exhibits at least 85% sequence identity to SEQ ID NO: 288, 289 or 291;
dd) the variable heavy chain set forth in SEQ ID NO: 235 or 237 or a sequence of amino acids that exhibits at least 85% sequence identity to SEQ ID NO: 235 or 237, and the variable light chain set forth in SEQ ID NO: 295, 296 or 298 or a sequence of amino acids that exhibits at least 85% sequence identity to SEQ ID NO: 295, 296 or 298;
ee) the variable heavy chain set forth in SEQ ID NO: 247 or 249 or a sequence of amino acids that exhibits at least 85% sequence identity to SEQ ID NO: 247 or 249, and the variable light chain set forth in SEQ ID NO: 302, 303 or 305 or a sequence of amino acids that exhibits at least 85% sequence identity to SEQ ID NO: 302, 303 or 305;
ff) the variable heavy chain set forth in SEQ ID NO: 211 or 213 or a sequence of amino acids that exhibits at least 85% sequence identity to SEQ ID NO: 211 or 213, and the variable light chain set forth in SEQ ID NO: 302, 303 or 305 or a sequence of amino acids that exhibits at least 85% sequence identity to SEQ ID NO: 302, 303 or 305;
gg) the variable heavy chain set forth in SEQ ID NO: 211 or 213 or a sequence of amino acids that exhibits at least 85% sequence identity to SEQ ID NO: 211 or 213, and the variable light chain set forth in SEQ ID NO: 281, 282 or 284 or a sequence of amino acids that exhibits at least 85% sequence identity to SEQ ID NO: 281, 282 or 284;
hh) the variable heavy chain set forth in SEQ ID NO: 211 or 213 or a sequence of amino acids that exhibits at least 85% sequence identity to SEQ ID NO: 211 or 213, and the variable light chain set forth in SEQ ID NO: 288, 289 or 291 or a sequence of amino acids that exhibits at least 85% sequence identity to SEQ ID NO: 288, 289 or 291.

14. The modified anti-EGFR antibody, or antigen-binding fragment thereof, of claim 1 that is an antigen-binding fragment selected from among Fab, Fab′, F(ab′)₂, single-chain Fv (scFv), Fv, dsFv, diabody, Fd and Fd′ fragments.

15. The modified anti-EGFR antibody, or antigen-binding fragment thereof, of claim 1 that is a full-length antibody.

16. The modified anti-EGFR antibody, or antigen-binding fragment thereof, of claim 15, wherein the modified anti-EGFR antibody comprises:
a) the heavy chain comprising the sequence of amino acids set forth in SEQ ID NO:
72, 76, 79, 82, 85, 88, 91, 94, 97, 100, 103, 106, 109, 112, 115, 118, or 121 or a variant thereof that exhibits at least 85%, 90%, or 95% sequence identity to SEQ ID NO: 72, 76, 79, 82, 85, 88, 91, 94, 97, 100, 103, 106, 109, 112, 115, 118, or 121; and
b) a light chain comprising the sequence of amino acids set forth in any of SEQ ID NOS: 3, 8, 10 or 13, or a variant thereof that exhibits at least 85%, 90% or 95% sequence identity thereto.

17. The anti-EGFR antibody, or antigen-binding fragment thereof, of claim 15, wherein the modified antibody comprises:
a heavy chain comprising the sequence of amino acids set forth in any of SEQ ID NOS: 72, 76, 79, 82, 85, 88, 91, 94, 97, 100, 103, 106, 109, 112, 115, 118, or 121, or a variant thereof that exhibits at least 85%, 90% or 95% sequence identity to any of SEQ ID NOS: 72, 76, 79, 82, 85, 88, 91, 94, 97, 100, 103, 106, 109, 112, 115, 118, or 121; and
a light chain comprising the sequence of amino acids set forth in SEQ ID NO: 124, or a variant thereof that exhibits at least 85%, 90% or 95% sequence identity to SEQ ID NO: 124.

18. The modified anti-EGFR antibody, or antigen-binding fragment thereof, of claim 3 that is an antigen-binding fragment selected from among Fab, Fab′, F(ab′)₂, single-chain Fv (scFv), Fv, dsFv, diabody, Fd and Fd′ fragments.

19. The modified anti-EGFR antibody, or antigen-binding fragment thereof, of claim 3 that is a full-length antibody.

20. The modified anti-EGFR antibody, or antigen-binding fragment thereof, of claim 19, wherein the antibody comprises:
a) a heavy chain comprising the sequence of amino acids set forth in SEQ ID NO: 59 or a sequence of amino acids that exhibits at least 85%, 90% or 95% sequence identity to SEQ ID NO: 59; and
b) the light chain comprising the sequence of amino acids set forth in SEQ ID NO: 181 or a sequence of amino acids that exhibits at least 85%, 90% or 95% sequence identity to SEQ ID NO: 181.

21. The modified anti-EGFR antibody, or antigen-binding fragment thereof, of claim 20, that comprises up to 1, 2, 3, 4, 5, 6, 7, 8, 9, or 10 amino acid replacements in the heavy chain set forth in SEQ ID NO: 59 and/or in the light chain set forth ins SEQ ID NO: 181, other than the replacement with E at a position corresponding to position 104 in the heavy chain.

22. The anti-EGFR antibody, or antigen-binding fragment thereof, of claim 19, wherein the antibody comprises a sequence of amino acids selected from among:
a) the heavy chain set forth in SEQ ID NO: 129 or a sequence of amino acids that exhibits at least 85%, 90% or 95% sequence identity to SEQ ID NO: 129, and the light chain set forth in SEQ ID NO: 153 or a sequence of amino acids that exhibits at least 85%, 90% or 95% sequence identity to SEQ ID NO: 153;
b) the heavy chain set forth in SEQ ID NO: 129 or a sequence of amino acids that exhibits at least 85%, 90% or 95% sequence identity to SEQ ID NO: 129, and the light chain set forth in SEQ ID NO: 160 or a sequence of amino acids that exhibits at least 85%, 90% or 95% sequence identity to SEQ ID NO: 160;
c) the heavy chain set forth in SEQ ID NO: 135 or a sequence of amino acids that exhibits at least 85%, 90% or 95% sequence identity to SEQ ID NO: 135, and the light chain set forth in SEQ ID NO: 153 or a sequence of amino acids that exhibits at least 85%, 90% or 95% sequence identity to SEQ ID NO: 153;
d) the heavy chain set forth in SEQ ID NO: 129 or a sequence of amino acids that exhibits at least 85%, 90% or 95% sequence identity to SEQ ID NO: 129, and the light chain set forth in SEQ ID NO: 167 or a sequence of amino acids that exhibits at least 85%, 90% or 95% sequence identity to SEQ ID NO: 167;
e) the heavy chain set forth in SEQ ID NO: 129 or a sequence of amino acids that exhibits at least 85%, 90% or 95% sequence identity to SEQ ID NO: 129, and the light chain set forth in SEQ ID NO: 174 or a sequence of amino acids that exhibits at least 85%, 90% or 95% sequence identity to SEQ ID NO: 174;
f) the heavy chain set forth in SEQ ID NO: 129 or a sequence of amino acids that exhibits at least 85%, 90% or 95% sequence identity to SEQ ID NO: 129, and the light chain set forth in SEQ ID NO: 181 or a sequence of amino acids that exhibits at least 85%, 90% or 95% sequence identity to SEQ ID NO: 181;
g) the heavy chain set forth in SEQ ID NO: 135 or a sequence of amino acids that exhibits at least 85%, 90% or 95% sequence identity to SEQ ID NO: 135, and the light chain set forth in SEQ ID NO: 181 or a sequence of amino acids that exhibits at least 85%, 90% or 95% sequence identity to SEQ ID NO: 181;
h) the heavy chain set forth in SEQ ID NO: 129 or a sequence of amino acids that exhibits at least 85%, 90% or 95% sequence identity to SEQ ID NO: 129, and the light chain set forth in SEQ ID NO: 188 or a sequence of amino acids that exhibits at least 85%, 90% or 95% sequence identity to SEQ ID NO: 188;
i) the heavy chain set forth in SEQ ID NO: 141 or a sequence of amino acids that exhibits at least 85%, 90% or 95% sequence identity to SEQ ID NO: 141, and the light chain set forth in SEQ ID NO: 181 or a sequence of amino acids that exhibits at least 85%, 90% or 95% sequence identity to SEQ ID NO: 181;
j) the heavy chain set forth in SEQ ID NO: 147 or a sequence of amino acids that exhibits at least 85%, 90% or 95% sequence identity to SEQ ID NO: 147, and the light chain set forth in SEQ ID NO: 195 or a sequence of amino acids that exhibits at least 85%, 90% or 95% sequence identity to SEQ ID NO: 195;
k) the heavy chain set forth in SEQ ID NO: 141 or a sequence of amino acids that exhibits at least 85%, 90% or 95% sequence identity to SEQ ID NO: 141, and the light chain set forth in SEQ ID NO: 195 or a sequence of amino acids that exhibits at least 85%, 90% or 95% sequence identity to SEQ ID NO: 195;
l) the heavy chain set forth in SEQ ID NO: 147 or a sequence of amino acids that exhibits at least 85%, 90% or 95% sequence identity to SEQ ID NO: 147, and the light chain set forth in SEQ ID NO: 202 or a sequence of amino acids that exhibits at least 85%, 90% or 95% sequence identity to SEQ ID NO: 202;
m) the heavy chain set forth in SEQ ID NO: 141 or a sequence of amino acids that exhibits at least 85%, 90% or 95% sequence identity to SEQ ID NO: 141, and the light chain set forth in SEQ ID NO: 202 or a sequence of amino acids that exhibits at least 85%, 90% or 95% sequence identity to SEQ ID NO: 202;
n) the heavy chain set forth in SEQ ID NO: 209 or a sequence of amino acids that exhibits at least 85%, 90% or 95% sequence identity to SEQ ID NO: 209, and the light chain set forth in SEQ ID NO: 251 or a sequence of amino acids that exhibits at least 85%, 90% or 95% sequence identity to SEQ ID NO: 251;
o) the heavy chain set forth in SEQ ID NO: 215 or a sequence of amino acids that exhibits at least 85%, 90% or 95% sequence identity to SEQ ID NO: 215, and the light chain set forth in SEQ ID NO: 251 or a sequence of amino acids that exhibits at least 85%, 90% or 95% sequence identity to SEQ ID NO: 251;
p) the heavy chain set forth in SEQ ID NO: 221 or a sequence of amino acids that exhibits at least 85%, 90% or 95% sequence identity to SEQ ID NO: 221, and the light chain set forth in SEQ ID NO: 258 or a sequence of amino acids that exhibits at least 85%, 90% or 95% sequence identity to SEQ ID NO: 258;
q) the heavy chain set forth in SEQ ID NO: 227 or a sequence of amino acids that exhibits at least 85%, 90% or 95% sequence identity to SEQ ID NO: 227, and the light chain set forth in SEQ ID NO: 258 or a sequence of amino acids that exhibits at least 85%, 90% or 95% sequence identity to SEQ ID NO: 258;
r) the heavy chain set forth in SEQ ID NO: 233 or a sequence of amino acids that exhibits at least 85%, 90% or 95% sequence identity to SEQ ID NO: 233, and the light chain set forth in SEQ ID NO: 265 or a sequence of amino acids that exhibits at least 85%, 90% or 95% sequence identity to SEQ ID NO: 265;
s) the heavy chain set forth in SEQ ID NO: 239 or a sequence of amino acids that exhibits at least 85%, 90% or 95% sequence identity to SEQ ID NO: 239, and the light chain set forth in SEQ ID NO: 272 or a sequence of amino acids that exhibits at least 85%, 90% or 95% sequence identity to SEQ ID NO: 272;
t) the heavy chain set forth in SEQ ID NO: 221 or a sequence of amino acids that exhibits at least 85%, 90% or 95% sequence identity to SEQ ID NO: 221, and the light chain set forth in SEQ ID NO: 272 or a sequence of amino acids that exhibits at least 85%, 90% or 95% sequence identity to SEQ ID NO: 272;
u) the heavy chain set forth in SEQ ID NO: 227 or a sequence of amino acids that exhibits at least 85%, 90% or 95% sequence identity to SEQ ID NO: 227, and the light chain set forth in SEQ ID NO: 272 or a sequence of amino acids that exhibits at least 85%, 90% or 95% sequence identity to SEQ ID NO: 272;
v) the heavy chain set forth in SEQ ID NO: 233 or a sequence of amino acids that exhibits at least 85%, 90% or 95% sequence identity to SEQ ID NO: 233, and the light chain set forth in SEQ ID NO: 279 or a sequence of amino acids that exhibits at least 85%, 90% or 95% sequence identity to SEQ ID NO: 279;
w) the heavy chain set forth in SEQ ID NO: 245 or a sequence of amino acids that exhibits at least 85%, 90% or 95% sequence identity to SEQ ID NO: 245, and the light chain set forth in SEQ ID NO: 279 or a sequence of amino acids that exhibits at least 85%, 90% or 95% sequence identity to SEQ ID NO: 279;
x) the heavy chain set forth in SEQ ID NO: 221 or a sequence of amino acids that exhibits at least 85%, 90% or 95% sequence identity to SEQ ID NO: 221, and the light chain set forth in SEQ ID NO: 279 or a sequence of amino acids that exhibits at least 85%, 90% or 95% sequence identity to SEQ ID NO: 279;
y) the heavy chain set forth in SEQ ID NO: 227 or a sequence of amino acids that exhibits at least 85%, 90% or 95% sequence identity to SEQ ID NO: 227, and the light chain set forth in SEQ ID NO: 279 or a sequence of amino acids that exhibits at least 85%, 90% or 95% sequence identity to SEQ ID NO: 279;
z) the heavy chain set forth in SEQ ID NO: 233 or a sequence of amino acids that exhibits at least 85%, 90% or 95% sequence identity to SEQ ID NO: 233, and the light chain set forth in SEQ ID NO: 286 or a sequence of amino acids that exhibits at least 85%, 90% or 95% sequence identity to SEQ ID NO: 286;
aa) the heavy chain set forth in SEQ ID NO: 245 or a sequence of amino acids that exhibits at least 85%, 90% or 95% sequence identity to SEQ ID NO: 245, and the light chain set forth in SEQ ID NO: 286 or a sequence of amino acids that exhibits at least 85%, 90% or 95% sequence identity to SEQ ID NO: 286;
bb) the heavy chain set forth in SEQ ID NO: 221 or a sequence of amino acids that exhibits at least 85%, 90% or 95% sequence identity to SEQ ID NO: 221, and the light chain set forth in SEQ ID NO: 286 or a sequence of amino acids that exhibits at least 85%, 90% or 95% sequence identity to SEQ ID NO: 286;
cc) the heavy chain set forth in SEQ ID NO: 227 or a sequence of amino acids that exhibits at least 85%, 90% or 95% sequence identity to SEQ ID NO: 227, and the light chain set forth in SEQ ID NO: 286 or a sequence of amino acids that exhibits at least 85%, 90% or 95% sequence identity to SEQ ID NO: 286;
dd) the heavy chain set forth in SEQ ID NO: 233 or a sequence of amino acids that exhibits at least 85%, 90% or 95% sequence identity to SEQ ID NO: 233, and the light chain set forth in SEQ ID NO: 293 or a sequence of amino acids that exhibits at least 85%, 90% or 95% sequence identity to SEQ ID NO: 293;
ee) the heavy chain set forth in SEQ ID NO: 245 or a sequence of amino acids that exhibits at least 85%, 90% or 95% sequence identity to SEQ ID NO: 245, and the light chain set forth in SEQ ID NO: 300 or a sequence of amino acids that exhibits at least 85%, 90% or 95% sequence identity to SEQ ID NO: 300;
ff) the heavy chain set forth in SEQ ID NO: 209 or a sequence of amino acids that exhibits at least 85%, 90% or 95% sequence identity to SEQ ID NO: 209, and the light chain set forth in SEQ ID NO: 300 or a sequence of amino acids that exhibits at least 85%, 90% or 95% sequence identity to SEQ ID NO: 300;
gg) the heavy chain set forth in SEQ ID NO: 209 or a sequence of amino acids that exhibits at least 85%, 90% or 95% sequence identity to SEQ ID NO: 209, and the light chain set forth in SEQ ID NO: 279 or a sequence of amino acids that exhibits at least 85%, 90% or 95% sequence identity to SEQ ID NO: 279; and
hh) the heavy chain set forth in SEQ ID NO: 209 or a sequence of amino acids that exhibits at least 85%, 90% or 95% sequence identity to SEQ ID NO: 209, and the light chain set forth in SEQ ID NO: 286 or a sequence of amino acids that exhibits at least 85%, 90% or 95% sequence identity to SEQ ID NO: 286.

23. The modified anti-EGFR antibody of claim 1, wherein the antibody exhibits a ratio of binding activity for EGFR of greater than 2.0 in the presence of a pH that is pH 6.0 to 6.5, inclusive compared to in the presence of or about pH 7.4, when measured under the same conditions except for the difference in pH.

24. The modified anti-EGFR antibody of claim 3, wherein the antibody exhibits a ratio of binding activity for EGFR of greater than 2.0 in the presence of a pH that is pH 6.0 to 6.5, inclusive compared to in the presence of or about pH 7.4, when measured under the same conditions except for the difference in pH.

25. The anti-EGFR antibody, or antigen-binding fragment, of claim 1 that is isolated or purified.

26. A conjugate, comprising an anti-EGFR antibody, or antigen-binding fragment thereof, of claim 1 linked directly or indirectly via a linker to a targeted agent.

27. A conjugate, comprising an anti-EGFR antibody, or antigen-binding fragment thereof, of claim 3 linked directly or indirectly via a linker to a targeted agent.

28. The conjugate of claim 27, wherein the conjugate has the following formula:
Ab-[(L)_{q}-(targeted agent)ₘ], wherein:
Ab is the anti-EGFR antibody or antigen-binding fragment thereof that binds to EGFR;
L is a linker for linking the Ab to the targeted agent;
m is at least 1;
q is 0 or more as long as the resulting conjugate binds to the EGFR;
the resulting conjugate binds to the EGFR.

29. The conjugate of claim 27, wherein the targeted agent is a therapeutic moiety.

30. The conjugate of claim 29, wherein the therapeutic moiety is a cytotoxic moiety, a radioisotope, a chemotherapeutic agent, a lytic peptide or a cytokine.

31. The conjugate of claim 29, wherein the therapeutic moiety is selected from among taxol; cytochalasin B; gramicidin D; ethidium bromide; emetine; mitomycin; etoposide; teniposide; vincristine; vinblastine; colchicine; doxorubicin; daunorubicin; dihydroxy anthracin dione; maytansine or an analog or derivative thereof; an auristatin or a functional peptide analog or derivative thereof; dolastatin 10 or 15 or an analogue thereof; irinotecan or an analogue thereof; mitoxantrone; mithramycin; actinomycin D; 1-dehydrotestosterone; a glucocorticoid; procaine; tetracaine; lidocaine; propranolol; puromycin; calicheamicin or an analog or derivative thereof; an antimetabolite; an alkylating agent; a platinum derivative; duocarmycin A, duocarmycin SA, rachelmycin (CC-1065), or an analog or derivative thereof; an antibiotic; pyrrolo[2,1-c][1, 4]-benzodiazepines (PBD); a toxin; ribonuclease (RNase); DNase I, Staphylococcal enterotoxin A; and pokeweed antiviral protein.

32. The conjugate of claim 31, wherein the therapeutic moiety is a maytansine derivative that is a maytansinoid selected from among ansamitocin and mertansine (DM1).

33. The conjugate of claim 32, wherein the linker L is selected from among N-succinimidyl-4(2-pyridylthio)propanoate (SPDP), succinimidyl-4-(N-maleimidomethyl)cyclohexane-1-carboxylate (SMCC) and N-succinimidyl-4-(2-pyridylthio)pentanoate (SPP).

34. The conjugate of claim 32, wherein:
the maytansinoid is DM1; and
the linker is succinimidyl-4-(N-maleimidomethyl)cyclohexane-1-carboxylate (SMCC).

35. The conjugate of claim of claim 31, wherein the therapeutic moiety is an auristatin or a functional peptide analog or derivative thereof that is monomethyl auristatin E (MMAE) or F (MMAF).

36. The conjugate of claim 35, wherein the linker comprises a group capable of reacting with a sulfhydryl group of the antibody.

37. The conjugate of claim 35, wherein the linker comprises a maleimide group or a bis-thiol alkylating reagent.

38. The conjugate of claim 37, wherein the linker comprises 4-(3-tosyl-2-(tosylmethyl)propanoyl)benzamide, maleimidocaproyl or maleimidopropanoyl (MP).

39. The conjugate of claim 35, wherein:
the targeted agent is MMAF;
the linker comprises maleimidocaproyl (MC); and
the (L)-(targeted agent) has the following structure:

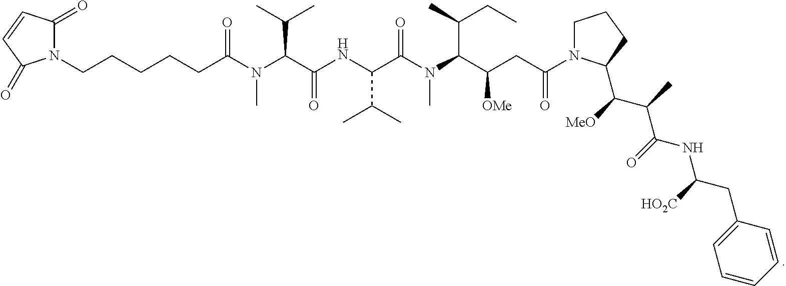 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)C=CC1=O)C(C)C]

40. The conjugate of claim 35, wherein:
the targeted agent is MMAE;
the linker comprises 6-maleimidocaproyl-valine-citrulline-p-aminobenzyloxycarbonyl (MC-vc-PAB); and
(L)-(targeted agent) has the following structure:

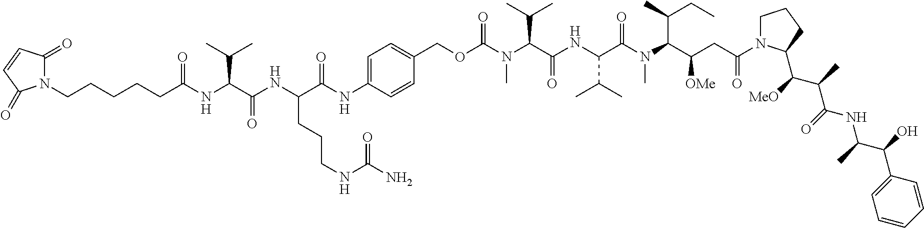 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)C(CCCNC(N)=O)NC(=O)[C@@H](NC(=O)CCCCCN2C(=O)C=CC2=O)C(C)C)cc1)C(C)C]

41. The conjugate of claim 35, wherein:
the targeted agent is MMAE;
the linker comprises 4-(3-tosyl-2-(tosylmethyl)propanoyl)benzamide-valine-citruline-p-aminobenzyloxycarbonyl; and
(L)-(targeted agent) has the following structure:

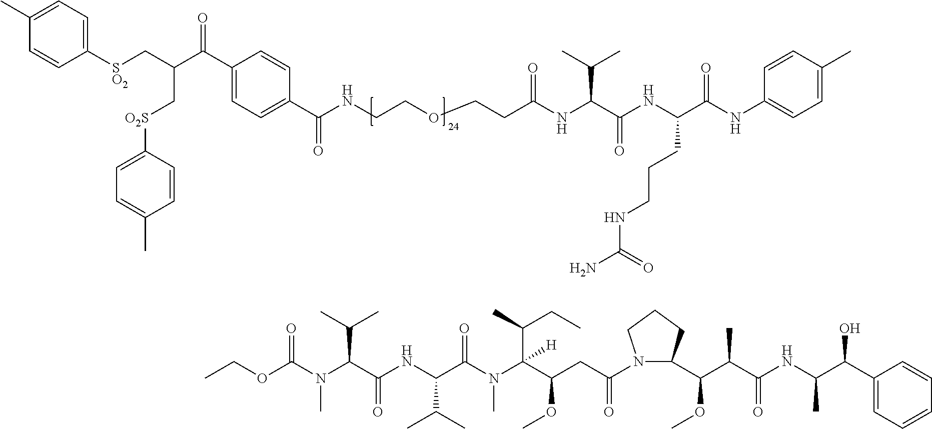

42. A nucleic acid molecule(s), selected from among:
a) a nucleic acid molecule comprising a sequence of nucleotides encoding an anti-EGFR antibody, or antigen-binding fragment thereof, of claim 1; and
b) a nucleic acid molecule, comprising a sequence of nucleotides encoding the heavy chain of any of the anti-EGFR antibody, or antigen-binding fragment thereof, of claim 1.

43. A vector, comprising the nucleic acid molecule(s) of claim 42.

44. A cell, comprising a vector or vectors of claim 43.

45. A method of making an anti-EGFR antibody, or antigen-binding fragment thereof, comprising expressing the heavy chain or light chain encoded from a vector or vectors of claim 43 encoding the heavy chain and the light chain in a suitable host cell and recovering the antibody.

46. A pharmaceutical composition comprising an anti-EGFR antibody or antigen-binding fragment of claim 1, and a pharmaceutically acceptable carrier or excipient.

47. A pharmaceutical composition comprising an anti-EGFR antibody or antigen-binding fragment of claim 3, and a pharmaceutically acceptable carrier or excipient.

48. A pharmaceutical composition comprising a conjugate of claim 27, and a pharmaceutically acceptable carrier or excipient.

49. The pharmaceutical composition of claim 46, comprising a stabilizing agent that is selected from among an amino acid, an amino acid derivative, an amine, a sugar, a polyols, a salt and a surfactant.

50. The pharmaceutical composition of claim 48, comprising a stabilizing agent that is selected from among an amino acid, an amino acid derivative, an amine, a sugar, a polyols, a salt and a surfactant.

51. The pharmaceutical composition of claim 50, wherein the stabilizing is a sugar or polyol selected from among glycerol, sorbitol, mannitol, inositol, sucrose and trehalose.

52. The pharmaceutical composition of claim 51, wherein the stabilizing agent is sucrose or trehalose.

53. The pharmaceutical composition of claim 50, wherein the stabilizing agent is a surfactant selected from among a polypropylene glycol, polyethylene glycol, glycerin, sorbitol, poloxamer and polysorbate.

54. The pharmaceutical composition of claim 53, wherein the surfactant is selected from among poloxamer 188, polysorbate 20 and polysorbate 80.

55. The pharmaceutical composition of claim 54, wherein the surfactant is polysorbate 80.

56. A method of treating a condition responsive to treatment with an anti-EGFR antibody in a subject, comprising administering to the subject a pharmaceutically effective amount of the pharmaceutical composition of claim 46.

57. A method of treating a condition responsive to treatment with an anti-EGFR antibody in a subject, comprising administering to the subject a pharmaceutically effective amount of the pharmaceutical composition of claim 47.

58. A method of treating a condition responsive to treatment with an anti-EGFR antibody in a subject, comprising administering to the subject a pharmaceutically effective amount of the pharmaceutical composition of claim 48.

59. The method of claim 58, wherein the condition responsive to treatment with an anti-EGFR antibody is a tumor, cancer or metastasis.

60. The method of claim 59, wherein the subject has a tumor that does not comprise a marker that confers resistance to anti-EGFR therapy.

61. The method of claim 60, wherein the marker is a mutation in KRAS, NRAS or BRAF.

62. An anti-EGFR antibody, or antigen-binding fragment thereof, comprising a variable heavy chain comprising amino acids 1-119 of the sequence set forth in SEQ ID NO: 57 and a variable light chain comprising the sequence of amino acids 1-107 of the sequence set forth in SEQ ID NO: 181, wherein the antibody comprises an asparagine (D) residue at the position corresponding to position 104 in SEQ ID NO: 57.

63. The modified anti-EGFR antibody, or antigen-binding fragment thereof, of claim 62 selected from among a full length antibody, Fab, Fab′, F(ab′)₂, single-chain Fv (scFv), Fv, dsFv, diabody, Fd and Fd′ fragments.

64. The modified anti-EGFR antibody, or antigen-binding fragment thereof, of claim 62, comprising a heavy chain comprising the sequence of amino acids set forth in SEQ ID NO: 57 and a light chain comprising the sequence of amino acids set forth in SEQ ID NO: 181, wherein the antibody comprises an asparagine (D) residue at the position corresponding to position 104 in SEQ ID NO: 57.

65. A conjugate, comprising an anti-EGFR antibody, or antigen-binding fragment thereof, of claim 62 linked directly or indirectly via a linker to a targeted agent.

66. A pharmaceutical composition comprising an anti-EGFR antibody or antigen-binding fragment of claim 62, and a pharmaceutically acceptable carrier or excipient.

67. A pharmaceutical composition comprising an anti-EGFR antibody or antigen-binding fragment of claim 65, and a pharmaceutically acceptable carrier or excipient.

68. A method of treating a condition responsive to treatment with an anti-EGFR antibody in a subject, comprising administering to the subject a pharmaceutically effective amount of the pharmaceutical composition of claim 66.

69. A method of treating a condition responsive to treatment with an anti-EGFR antibody in a subject, comprising administering to the subject a pharmaceutically effective amount of the pharmaceutical composition of claim 67.
